(19) United States
(12) Patent Application Publication
(10) Pub. No.: US 2025/0382305 A1
(43) Pub. Date: Dec. 18, 2025

(54) COMPOUNDS FOR TREATING SPINOCEREBELLAR ATAXIA TYPE 3

(71) Applicant: PTC THERAPEUTICS, INC., Warren, NJ (US)

(72) Inventors: Michael A. ARNOLD, Flemington, NJ (US); James R. ANNAND, Hillsborough, NJ (US); Gayan Mirihana ARACHCHILAGE, Budd Lake, NJ (US); Anuradha BHATTACHARYYA, Edison, NJ (US); Guangming CHEN, Bridgewater, NJ (US); Jackie CHIU, Hillsborough, NJ (US); Gaurav DAHIYA, Somerset, NJ (US); Yao JIANG, Raritan, NJ (US); Arun Raj KIZHAKKAYIL MANGADAN, Metuchen, NJ (US); Christie MORRILL, Green Brook, NJ (US); Stephen E. MOTIKA, New Brunswick, NJ (US); Jana NARASIMHAN, Scotch Plains, NJ (US); Matthew G. WOLL, Dunellen, NJ (US); Nanjing ZHANG, Princeton, NJ (US); Xiaoyan ZHANG, Belle Mead, NJ (US)

(21) Appl. No.: 18/877,505

(22) PCT Filed: Jun. 20, 2023
(86) PCT No.: PCT/US2023/068717

§ 371 Date: Dec. 20, 2024

Related U.S. Application Data
(60) Provisional application No. 63/354,339, filed on Jun. 22, 2022.

(51) Int. Cl. C07D 495/04; A61K 31/4365; A61K 31/444; A61K 31/4545; A61K 31/519; A61K 31/5377; A61K 31/55; C07B 59/00; C07D 487/04

(52) U.S. Cl. CPC C07D 495/04; A61K 31/4365; A61K 31/444; A61K 31/4545; A61K 31/519; A61K 31/5377; A61K 31/55; C07B 59/002; C07D 487/04

(57) ABSTRACT

An aspect of the present description relates to compounds useful for improving pre-mRNA splicing in a cell. In particular, another aspect of the present description relates to substituted heteroaryl compounds, forms, and pharmaceutical compositions thereof and methods of use for treating or ameliorating spinocerebellar ataxia type 3 (SCA3), also known as Machado-Joseph disease (MJD).

### CROSS-REFERENCE TO RELATED APPLICATIONS

This application is an International Application claiming benefit of U.S. Provisional Patent Application No. 63/354,339, filed Jun. 22, 2022, entitled COMPOUNDS FOR TREATING SPINOCEREBELLAR ATAXIA TYPE 3, the contents of which are herein incorporated by reference in its entirety for all purposes.

### TECHNICAL FIELD

An aspect of the present description relates to compounds useful for improving pre-mRNA splicing in a cell. In particular, another aspect of the present description relates to substituted heteroaryl compounds, forms, and pharmaceutical compositions thereof and methods of use for treating or ameliorating spinocerebellar ataxia type 3 (SCA3), also known as Machado-Joseph disease (MJD).

### BACKGROUND

Spinocerebellar ataxia type 3 (SCA3), also known as Machado-Joseph disease (MID), is a rare autosomal dominantly inherited disease characterized by progressive ataxia. SCA3 is the most common dominant ataxia worldwide. Although the accurate patient population is unknown, it has been estimated that the average prevalence is 1-5/100,000 with higher frequency in China, Portugal, Brazil, Netherlands, Germany, and Japan. It is also significant in the United States wherein SCA3 accounts for ˜21% of dominant ataxia. Based on the age of onset, there are three subtypes of SCA3: subtype 1 (early-onset, 10-30 yr), subtype 2 (average-onset, 30-50 yr), and subtype 3 (late-onset, 50-70 yr). The SCA3 patients usually survive 10 to 20 years after the onset of symptoms. Symptoms include slowly progressive clumsiness in the arms and legs, a staggering lurching gait that can be mistaken for drunkenness, difficulty with speech and swallowing, impaired eye movements sometimes accompanied by double vision or bulging eyes, and lower limb spasticity; some individuals develop sustained muscle contractions that cause twisting of the body and limbs, repetitive movements, and abnormal postures; and others may develop twitching of the face or tongue, neuropathy, or problems with urination and the autonomic nervous system.

SCA3 is caused by an unstable expansion of cytosine-adenine-guanine (CAG) trinucleotide repeats in the ATXN3 gene that transcribes into mutant ATXN3 (mATXN3) mRNA. This expansion in the mATXN3 mRNA leads to production of mutant ataxin-3 protein (ATXN3) containing a polymorphic polyglutamine (polyQ) tract. Both the mATXN3 mRNA and the mutant ATXN3 protein disrupt several cellular processes resulting in neurodegeneration in the cerebellum, brainstem, and other connected brain regions.

The number of CAG repeats in the ATXN3 mRNA ranges 10-45 in the healthy population, whereas in SCA3 patients, it can vary from 61-87. The number of CAG repeats between 45-60 is associated with an incomplete penetrance of the disease. As evidenced in other polyQ disorders, the number of repeats inversely correlates with the age of onset in SCA3 patients.

In several preclinical models of SCA3, reduction of ATXN3 protein levels improves SCA3 pathology, thus confirming the importance of ATNX3 lowering as a therapeutic target to ameliorate the downstream pathogenic effects. The present description relates to the use of a compound of Formula (I) or a form or composition thereof for treating SCA3. These sets of compounds induce exon 4 skipping in the ATXN3 pre-mRNA during the splicing process. Exon 4 skipping of ATXN3 mRNA changes the open reading frame (ORF) and creates premature termination codons (PTCs) in the ATXN3 exon 4-skipped mRNA (ΔE4 mRNA). It has been shown that such exon skipping splicing events could serve to reduce gene expression by creating mRNAs with premature termination codons, thus signaling the mRNAs to be degraded rather than translated into proteins. Similarly, ATXN3 ΔE4 mRNA produced in the presence of these compounds will undergo mRNA degradation resulting in decreased levels of ATXN3 mRNA, resulting in ATXN3 protein lowering.

To date, there are no disease-modifying therapies available for SCA3, and there exists a need for improved methods and compositions for treating SCA3 and the symptoms associated therewith. The compounds described herein represent potential ATXN3 pre-mRNA splicing compounds that could be used as a disease-modifying treatment for SCA3.

All other documents referred to herein are incorporated by reference into the present application as though fully set forth herein.

### SUMMARY

An aspect of the present description includes compounds of Formula (I):

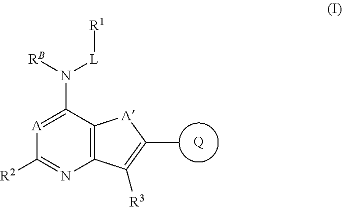

or a form thereof, wherein A, A′, L, R^{B}, R¹, R², R³, and Ring Q are defined herein.

An aspect of the present description includes a method for use of a compound of Formula (I) or a form or composition thereof for treating or ameliorating SCA3 in a subject in need thereof comprising, administering to the subject an effective amount of the compound of Formula (I) or a form or composition thereof.

An aspect of the present description includes a use for a compound of Formula (I) or a form thereof for treating or ameliorating SCA3 in a subject in need thereof comprising, administering to the subject an effective amount of the compound of Formula (I) or a form thereof.

An aspect of the present description includes a use for a compound of Formula (I) or a form thereof in the manufacture of a medicament for treating or ameliorating SCA3 in a subject in need thereof comprising, administering to the subject an effective amount of the medicament.

### DETAILED DESCRIPTION

An aspect of the present description relates to compounds of Formula (I):

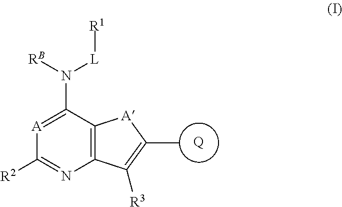

or a form thereof, wherein:
   A is selected from the group consisting of CR^{A} and N;
   A′ is selected from the group consisting of S and NR^{A′};
   L is selected from the group consisting of CH₂ and CD₂;
   R^{A} is selected from the group consisting of hydrogen, halo, C₁₋₆alkyl, and C₃₋₈cycloalkyl;
   R^{A′} is selected from the group consisting of hydrogen, C₁₋₄alkyl, and halo-C₁₋₄alkyl;
   R^{B} is selected from the group consisting of hydrogen and C₁₋₆alkyl;
   R¹ is selected from the group consisting of phenyl, heteroaryl, C₃₋₈cycloalkyl, CO₂C₁₋₆alkyl, C₂₋₆alkenyl, and C₂₋₆alkynyl;
   wherein heteroaryl is a 5-11 membered monocyclic or bicyclic aromatic carbon atom ring structure radical containing 1-3 heteroatoms selected from N, O, and S;
   wherein C₃₋₈cycloalkyl is a saturated or partially unsaturated monocyclic or bicyclic ring system;
   wherein phenyl heteroaryl, and C₃₋₈cycloalkyl are substituted with zero, one, two, three, or four, independently selected R^{1a} substituents,
   R^{1a} is selected from the group consisting of cyano, halo, hydroxy, C₁₋₆alkyl, halo-C₁₋₆alkyl, deutero-C₁₋₆alkyl, and C₁₋₆alkoxy;
   R² is selected from the group consisting of hydrogen, cyano, halo, C₂₋₆alkynyl, C₁₋₆alkoxy-C₂₋₆alkynyl, and hydroxy-C₂₋₆alkynyl;
   R³ is selected from the group consisting of hydrogen, cyano, halo, hydroxy, SH, C₁₋₆alkyl, halo-C₁₋₆alkyl, hydroxy-C₁₋₆alkyl, C₁₋₆alkoxy, halo-C₁₋₆alkoxy, thio-C₁₋₆alkyl, C₂₋₆alkenyl, C₂₋₆alkynyl, C₁₋₆alkoxy-C₂₋₆alkynyl, hydroxy-C₂₋₆alkynyl, (CH₃)₃Si—C₂₋₆alkynyl, heteroaryl-C₂₋₆alkynyl, C₃₋₈cycloalkyl, phenyl, and heteroaryl,
   wherein heteroaryl is a 5-11 membered monocyclic or bicyclic aromatic carbon atom ring structure radical containing 1-3 heteroatoms selected from N, O, and S, and
   wherein each instance of C₃₋₈cycloalkyl, phenyl, and heteroaryl are independently substituted with zero, one, two, three, or four, independently selected R^{3a} substituents;
   R^{3a} is selected from the group consisting of cyano, halo, hydroxy, C₁₋₆alkyl, halo-C₁₋₆alkyl, deutero-C₁₋₆alkyl, and C₁₋₆alkoxy;
Ring Q is

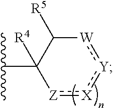

R⁴ is selected from the group consisting of hydrogen, cyano, halo, hydroxy, C₁₋₆alkyl, halo-C₁₋₆alkyl, C₁₋₆alkoxy, and halo-C₁₋₆alkoxy;
R⁵ is selected from the group consisting of amino, C₁₋₄alkyl-amino, and (C₁₋₄alkyl)₂-amino;
W is selected from the group consisting of CH₂, CD₂, CH—R^{w}, CD-R^{w}, C(R^{w})₂, and C(O);
X is selected from the group consisting of CH₂, CD₂, CH—R^{X}, CD-R^{X}, C(R^{X})₂, CH, CD, CR^{X}, C═CH₂, C═CD₂, C═C(R^{X})₂, C(O), NH, N—C₁₋₄alkyl, N-phenyl, O, S, S(O), and SO₂;
Y is selected from the group consisting of CH₂, CD₂, CH—R^{Y}, CD-R^{Y}, C(R^{Y})₂, CH, CD, CR^{Y}, C═CH₂, C═CD₂, C═C(R^{Y})₂, N-phenyl, O, S, S(O), and SO₂;
Z is selected from the group consisting of CH₂, CD₂, CH—R^{Z}, CD-R^{Z}, C(R^{Z})₂, CH, CD, CR^{Z}, NH, N—C₁₋₄alkyl, N-phenyl, O, S, S(O), and SO₂;
each R^{W}, R^{X}, R^{Y}, R^{Z} are independently selected from the group consisting of halo, hydroxy, C₁₋₆alkyl, halo-C₁₋₆alkyl, C₁₋₆alkoxy, halo-C₁₋₆alkoxy, amino, C₁₋₄alkyl-amino, (C₁₋₄alkyl)₂-amino, CO₂H, CO₂C₁₋₆alkyl, C(O)NH₂, C(O)N(C₁₋₆alkyl)₂, C(O)-heterocyclyl, and C(O)NH-phenyl, and wherein each R^{W}, R^{X}, R^{Y}, R^{Z} may combine to form a carbocyclic or heterocyclic ring;
n is selected from the group consisting of 0, 1, 2, and 3; and
[symbol] independently represents a single bond or a double bond as valency permits;
   wherein a form of the compound is a pharmaceutically acceptable salt, hydrate, solvate, racemate, enantiomer, diastereomer, stereoisomer, tautomer or isotope enriched form thereof.

One aspect includes a compound of Formula (I), wherein the compound is a compound of Formula (Ia) or a form thereof

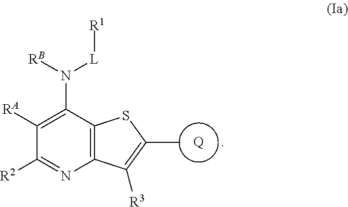

Another aspect includes a compound of Formula (Ia) or a form thereof, wherein
L is CH₂ or CD₂;
R^{A} is hydrogen;
R^{B} is hydrogen or CH₃; and
R² is Cl or CN.

One aspect includes a compound of Formula (I), wherein the compound is a compound of Formula (Ib) or a form thereof

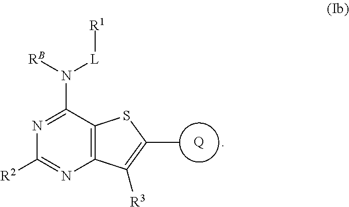

Another aspect includes a compound of Formula (Ib) or a form thereof, wherein
L is CH₂;
R^{B} is hydrogen; and
R² is Cl.

One aspect includes a compound of Formula (I), wherein the compound is a compound of Formula (Ic) or a form thereof

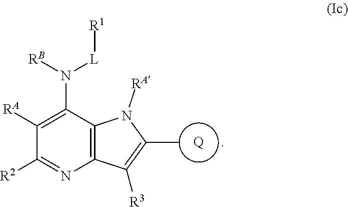

Another aspect includes a compound of Formula (Ic) or a form thereof, wherein
L is CH₂;
R^{A} is hydrogen;
R^{A′} is CH₃ or CHF₂;
R^{B} is hydrogen; and
R² is Cl.

One aspect includes a compound of Formula (I), wherein the compound is a compound of Formula (Id) or a form thereof

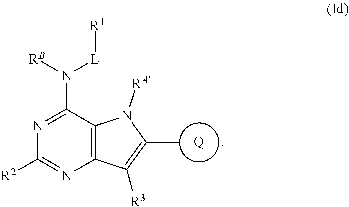

Another aspect includes a compound of Formula (Id) or a form thereof, wherein
L is CH₂;
R^{A′} is CHF₂:
R^{B} is hydrogen; and
R² is Cl.

One aspect includes a compound of Formula (I), wherein A is selected from the group consisting of CR^{A} and N.

Another aspect includes a compound of Formula (I), wherein A is CR^{A}.

Another aspect includes a compound of Formula (I), wherein A is N.

One aspect includes a compound of Formula (I), wherein A′ is selected from the group consisting of S and NR^{A′}.

Another aspect includes a compound of Formula (I), wherein A′ is S.

Another aspect includes a compound of Formula (I), wherein A′ is NR^{A′}.

One aspect includes a compound of Formula (I), wherein L is selected from the group consisting of CH₂ and CD₂.

One aspect includes a compound of Formula (I), wherein R^{A} is selected from the group consisting of hydrogen, halo, C₁₋₆alkyl, and C₃₋₈cycloalkyl.

Another aspect includes a compound of Formula (I), wherein R^{A} is hydrogen.

One aspect includes a compound of Formula (I), wherein R^{A′} is selected from the group consisting of hydrogen, C₁₋₄alkyl, and halo-C₁₋₄alkyl.

Another aspect includes a compound of Formula (I), wherein R^{A′} is selected from the group consisting of CH₃ and CHF₂.

One aspect includes a compound of Formula (I), wherein R^{B} is selected from the group consisting of hydrogen and C₁₋₆alkyl.

Another aspect includes a compound of Formula (I), wherein R^{B} is selected from the group consisting of hydrogen and CH₃.

One aspect includes a compound of Formula (I), wherein R¹ is selected from the group consisting of phenyl, heteroaryl, C₃₋₈cycloalkyl, CO₂C₁₋₆alkyl, C₂₋₆alkenyl, and C₂₋₆alkynyl;
wherein heteroaryl is a 5-11 membered monocyclic or bicyclic aromatic carbon atom ring structure radical containing 1-3 heteroatoms selected from N, O, and S;
wherein C₃₋₈cycloalkyl is a saturated or partially unsaturated monocyclic or bicyclic ring system;
wherein phenyl heteroaryl, and C₃₋₈cycloalkyl are substituted with zero, one, two, three, or four, independently selected R^{1a} substituents.

Another aspect includes a compound of Formula (I), wherein R¹ is phenyl substituted with zero, one, two, three, or four, independently selected R^{1a} substituents.

Another aspect includes a compound of Formula (I), wherein R¹ is unsubstituted phenyl.

Another aspect includes a compound of Formula (I), wherein R¹ is phenyl substituted with one R^{1a} substituent.

Another aspect includes a compound of Formula (I), wherein R¹ is heteroaryl substituted with zero, one, two, three, or four, independently selected R^{1a} substituents, wherein heteroaryl is a 5-11 membered monocyclic or bicyclic aromatic carbon atom ring structure radical containing 1-3 heteroatoms selected from N, O, and S.

Another aspect includes a compound of Formula (I), wherein R¹ is unsubstituted heteroaryl, wherein heteroaryl is a 5-11 membered monocyclic or bicyclic aromatic carbon atom ring structure radical containing 1-3 heteroatoms selected from N, O, and S.

Another aspect of includes a compound of Formula (I), wherein R¹ is heteroaryl selected from furanyl, thiophenyl, 1H-pyrazolyl, 1H-imidazolyl, isoxazolyl, 1,3-thiazolyl, 1,3-oxazolyl, tetrazolyl, 1,2,3-triazolyl, 1,2,4-oxadiazolyl, 1,3,4-oxadiazolyl, 1,2,3-thiadiazolyl, pyridinyl, pyrimidinyl, pyrazinyl, pyridazinyl, benzofuranyl, and quinolinyl, wherein heteroaryl is substituted with zero, one, two, three, or four, independently selected Ria substituents.

Another aspect of includes a compound of Formula (I), wherein R¹ is heteroaryl selected from furanyl, and thiophenyl, wherein heteroaryl is substituted with zero, one, two, three, or four, independently selected R^{1a} substituents.

Another aspect of includes a compound of Formula (I), wherein R¹ is unsubstituted heteroaryl selected from furanyl, thiophenyl, 1,3-thiazolyl, 1,3-oxazolyl, and pyridinyl.

Another aspect includes a compound of Formula (I), wherein R¹ is heteroaryl selected from furan-2-yl, furan-3-yl, thiophen-2-yl, thiophen-3-yl, 1H-pyrazol-1-yl, 1H-pyrazol-3-yl, 1H-pyrazol-4-yl, 1H-pyrazol-5-yl, 1H-imidazol-1-yl, 1H-imidazol-4-yl, isoxazol-3-yl, isoxazol-4-yl, isoxazol-5-yl, 1,3-thiazol-2-yl, 1,3-thiazol-4-yl, 1,3-thiazol-5-yl, 1,3-oxazol-2-yl, 1,3-oxazol-4-yl, 1,3-oxazol-5-yl, 1,2,4-oxadiazol-3-yl, 1,3,4-oxadiazol-2-yl, tetrazol-5-yl, 1,2,3-triazol-4-yl, 1,2,3-triazol-5-yl, 1,2,3-thiadiazol-4-yl, 1,2,3-thiadiazol-5-yl, pyridin-2-yl, pyridin-3-yl, pyridin-4-yl, pyrimidin-4-yl, pyrazin-2-yl, pyridazin-3-yl, pyridazin-4-yl, benzofuran-2-yl, benzofuran-5-yl, and quinoline-4-yl, wherein heteroaryl is substituted with zero, one, two, three, or four, independently selected R^{1a} substituents.

Another aspect includes a compound of Formula (I), wherein R¹ is heteroaryl selected from furan-2-yl, thiophen-2-yl, thiophen-3-yl, 1,3-thiazol-2-yl, 1,3-oxazol-2-yl, and pyridin-4-yl, wherein heteroaryl is substituted with zero, one, two, three, or four, independently selected R^{1a} substituents.

Another aspect of includes a compound of Formula (I), wherein R¹ is unsubstituted heteroaryl selected from furan-2-yl, thiophen-2-yl, thiophen-3-yl, 1,3-thiazol-2-yl, 1,3-oxazol-2-yl, and pyridin-4-yl.

One aspect includes a compound of Formula (I), wherein R¹ is CO₂C₁₋₆alkyl.

Another aspect includes a compound of Formula (I), wherein R¹ is CO₂CH₃.

One aspect includes a compound of Formula (I), wherein R¹ is C₃₋₈cycloalkyl, wherein C₃₋₈cycloalkyl is a saturated or partially unsaturated monocyclic or bicyclic ring system substituted with zero, one, two, three, or four, independently selected R^{1a} substituents.

Another aspect includes a compound of Formula (I), wherein R¹ is

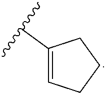 [SMILES: *C1=CCCC1]

One aspect includes a compound of Formula (I), wherein R¹ is C₂₋₆alkenyl.

Another aspect of includes a compound of Formula (I), wherein R¹ is

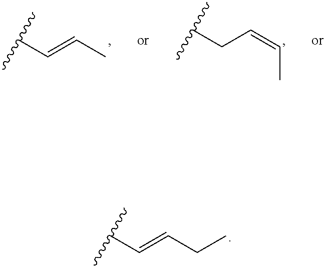

One aspect includes a compound of Formula (I), wherein R¹ is C₂₋₆alkynyl.

Another aspect of includes a compound of Formula (I), wherein R¹ is

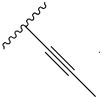 [SMILES: *C#CC]

Another aspect of includes a compound of Formula (I), wherein R¹ is phenyl, CO₂CH₃,

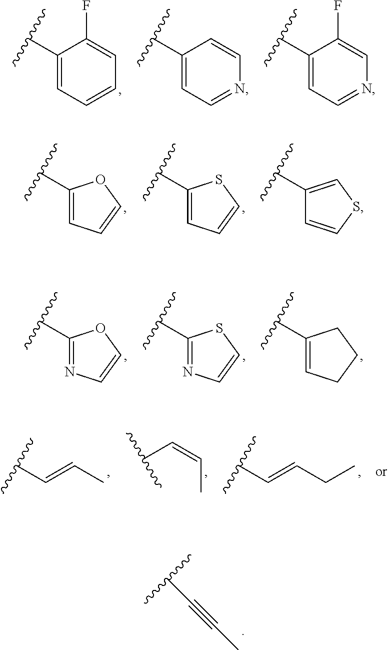

One aspect includes a compound of Formula (I), wherein R^{1a} is selected from the group consisting of cyano, halo, hydroxy, C₁₋₆alkyl, halo-C₁₋₆alkyl, deutero-C₁₋₆alkyl, and C₁₋₆alkoxy.

Another aspect includes a compound of Formula (I), wherein R^{1a} is halo.

Another aspect includes a compound of Formula (I), wherein R^{1a} is halo selected from fluoro, chloro, bromo, and iodo.

Another aspect includes a compound of Formula (I), wherein R^{1a} is fluoro.

One aspect includes a compound of Formula (I), wherein R² is selected from the group consisting of hydrogen, cyano, halo, C₂₋₆alkynyl, C₁₋₆alkoxy-C₂₋₆alkynyl, and hydroxy-C₂₋₆alkynyl.

Another aspect includes a compound of Formula (I), wherein R² is cyano.

Another aspect includes a compound of Formula (I), wherein R² is halo.

Another aspect includes a compound of Formula (I), wherein R² is halo selected from fluoro, chloro, bromo, and iodo.

Another aspect includes a compound of Formula (I), wherein R² is chloro.

One aspect includes a compound of Formula (I), wherein R³ is selected from the group consisting of hydrogen, cyano, halo, hydroxy, SH, C₁₋₆alkyl, halo-C₁₋₆alkyl, hydroxy-C₁₋₆alkyl, C₁₋₆alkoxy, halo-C₁₋₆alkoxy, thio-C₁₋₆alkyl, C₂₋₆alkenyl, C₂₋₆alkynyl, C₁₋₆alkoxy-C₂₋₆alkynyl, hydroxy-C₂₋₆alkynyl, (CH₃)₃Si—C₂₋₆alkynyl, heteroaryl-C₂₋₆alkynyl, C₃₋₈cycloalkyl, phenyl, and heteroaryl,
wherein heteroaryl is a 5-11 membered monocyclic or bicyclic aromatic carbon atom ring structure radical containing 1-3 heteroatoms selected from N, O, and S, and
wherein each instance of C₃₋₈cycloalkyl, phenyl, and heteroaryl are independently substituted with zero, one, two, three, or four, independently selected R^{3a} substituents.

Another aspect includes a compound of Formula (I), wherein R³ is selected from the group consisting of hydrogen, cyano, halo, C₁₋₆alkyl, halo-C₁₋₆alkyl, hydroxy-C₁₋₆alkyl, halo-C₁₋₆alkoxy, C₂₋₆alkenyl, C₂₋₆alkynyl, C₁₋₆alkoxy-C₂₋₆alkynyl, hydroxy-C₂₋₆alkynyl, (CH₃)₃Si—C₂₋₆alkynyl, heteroaryl-C₂₋₆alkynyl, C₃₋₈cycloalkyl, and phenyl,
wherein heteroaryl is a 5-11 membered monocyclic or bicyclic aromatic carbon atom ring structure radical containing 1-3 heteroatoms selected from N, O, and S, and
wherein C₃₋₈cycloalkyl, phenyl, and heteroaryl are independently substituted with zero, one, two, three, or four, independently selected R^{3a} substituents.

Another aspect includes a compound of Formula (I), wherein R³ is hydrogen.

Another aspect includes a compound of Formula (I), wherein R³ is cyano.

Another aspect includes a compound of Formula (I), wherein R³ is halo.

Another aspect includes a compound of Formula (I), wherein R³ is halo selected from fluoro, chloro, bromo, and iodo.

Another aspect includes a compound of Formula (I), wherein R³ is chloro.

Another aspect includes a compound of Formula (I), wherein R³ is bromo.

Another aspect includes a compound of Formula (I), wherein R³ is iodo.

Another aspect includes a compound of Formula (I), wherein R³ is C₁₋₆alkyl selected from methyl, ethyl, propyl, isopropyl, butyl, tert-butyl, pentyl, and hexyl.

Another aspect includes a compound of Formula (I), wherein R³ is methyl.

Another aspect includes a compound of Formula (I), wherein R³ is ethyl.

Another aspect includes a compound of Formula (I), wherein R³ is halo-C₁₋₆alkyl selected from the group consisting of CH₂F, CHF₂, CF₃, and CH₂CH₂F.

Another aspect includes a compound of Formula (I), wherein R³ is hydroxy-C₁₋₆alkyl selected from CH₂OH.

Another aspect includes a compound of Formula (I), wherein R³ is halo-C₁₋₆alkoxy selected from CH₂OCHF₂.

Another aspect includes a compound of Formula (I), wherein R³ is C₂₋₆alkenyl selected from CH═CH₂ and CH═C═CH₂.

Another aspect includes a compound of Formula (I), wherein R³ is C₂₋₆alkynyl selected from the group consisting of

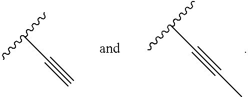

Another aspect includes a compound of Formula (I), wherein R³ is hydroxy-C₂₋₆alkynyl selected from the group consisting of

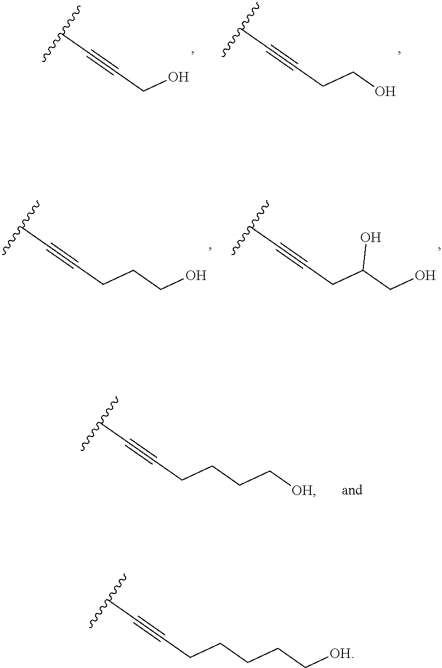 [SMILES: *C#CCO ; *C#CCCO ; *C#CCCCO ; *C#CCC(O)CO ; *C#CCCCCO ; *C#CCCCCCO]

Another aspect includes a Formula (I), wherein R³ is (CH₃)₃Si—C₂₋₆alkynyl selected from

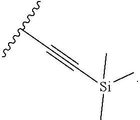 [SMILES: *C#C[Si](C)(C)C]

Another aspect includes a Formula (I), wherein R³ is heteroaryl-C₂₋₆alkynyl selected from

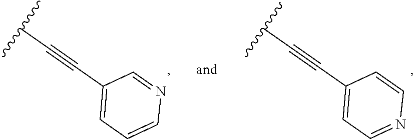 [SMILES: *C#Cc1cccnc1 ; *C#Cc1ccncc1]

wherein the heteroaryl ring is substituted with zero, one, two, three, or four, independently selected R^{3a} substituents.

Another aspect includes a compound of Formula (I), wherein R³ is C₃₋₈cycloalkyl selected from cyclopropyl, cyclobutyl, cyclopentyl, cyclohexyl, cycloheptyl, and cyclooctyl substituted with zero, one, two, three, or four, independently selected R^{3a} substituents.

Another aspect includes a compound of Formula (I), wherein R³ is cyclopropyl substituted with zero, one, two, three, or four, independently selected R^{3a} substituents.

Another aspect includes a compound of Formula (I), wherein R³ is unsubstituted cyclopropyl.

Another aspect includes a compound of Formula (I), wherein R³ is phenyl substituted with zero, one, two, three, or four, independently selected R^{3a} substituents.

Another aspect includes a compound of Formula (I), wherein R³ is unsubstituted phenyl.

One aspect includes a compound of Formula (I), wherein R^{3a} is selected from the group consisting of cyano, halo, hydroxy, C₁₋₆alkyl, halo-C₁₋₆alkyl, deutero-C₁₋₆alkyl, and C₁₋₆alkoxy.

Another aspect includes a compound of Formula (I), wherein R^{3a} is selected from the group consisting of Cl and OCH₃.

Another aspect includes a compound of Formula (I), wherein R³ is selected from the group consisting of hydrogen, cyano, C₁, Br, I, CH₃, CH₂CH₃, CHF₂, CF₃, CH₂CH₂F, CH₂OH, CH₂OCHF₂, CH═CH₂, CH═C═CH₂, cyclopropyl, phenyl,

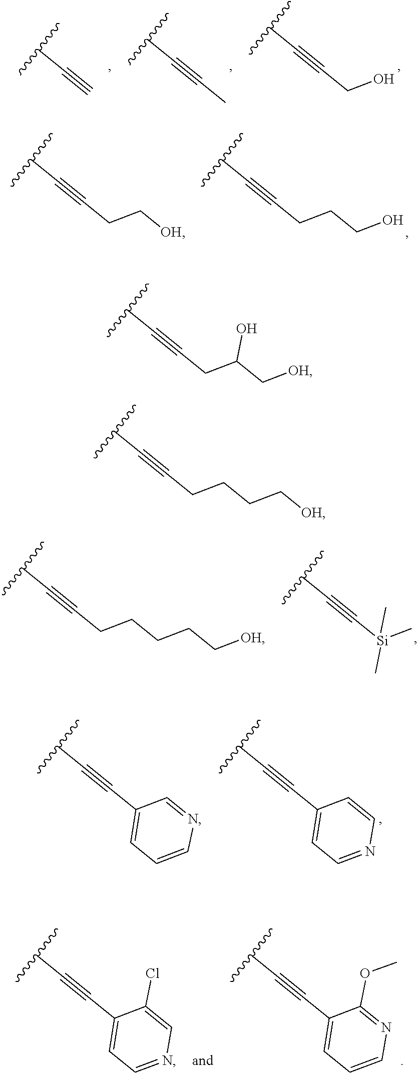

One aspect includes a compound of Formula (I), wherein Ring Q is

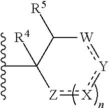

and any stereoisomers thereof.

One aspect includes a compound of Formula (I), wherein R⁴ is selected from the group consisting of hydrogen, cyano, halo, hydroxy, C₁₋₆alkyl, halo-C₁₋₆alkyl, C₁₋₆alkoxy, and halo-C₁₋₆alkoxy.

Another aspect includes a compound of Formula (I), wherein R⁴ is selected from the group consisting of hydrogen, halo, and hydroxy.

Another aspect includes a compound of Formula (I), wherein R⁴ is selected from the group consisting of hydrogen.

Another aspect includes a compound of Formula (I), wherein R⁴ is halo.

Another aspect includes a compound of Formula (I), wherein R⁴ is halo selected from fluoro, chloro, bromo, and iodo.

Another aspect includes a compound of Formula (I), wherein R⁴ is fluoro.

Another aspect includes a compound of Formula (I), wherein R⁴ is hydroxy.

One aspect includes a compound of Formula (I), wherein R⁵ is selected from the group consisting of amino, C₁₋₄alkyl-amino, and (C₁₋₄alkyl)₂-amino.

Another aspect includes a compound of Formula (I), wherein R⁵ is amino.

Another aspect includes a compound of Formula (I), wherein R⁵ is (C₁₋₄alkyl)₂-amino.

Another aspect includes a compound of Formula (I), wherein R⁵ is N₂(CH₃).

Another aspect includes a compound of Formula (I), wherein R⁵ is C₁₋₄alkyl-amino.

Another aspect includes a compound of Formula (I), wherein R⁵ is NH(CH₃).

One aspect includes a compound of Formula (I), wherein W is selected from the group consisting of CH₂, CD₂, CH—R^{w}, CD-R^{w}, C(R^{w})₂, and C(O).

Another aspect includes a compound of Formula (I), wherein W is CH₂.

Another aspect includes a compound of Formula (I), wherein W is CD₂.

Another aspect includes a compound of Formula (I), wherein W is C(O).

One aspect includes a compound of Formula (I), wherein X is selected from the group consisting of CH₂, CD₂, CH—R^{X}, CD-R^{X}, C(R^{X})₂, CH, CD, CR^{X}, C═CH₂, C═CD₂, C═C(R^{X})₂, C(O), NH, N—C₁₋₄alkyl, N-phenyl, O, S, S(O), and SO₂.

Another aspect includes a compound of Formula (I), wherein X is CH₂.

Another aspect includes a compound of Formula (I), wherein X is CH—R^{X}.

Another aspect includes a compound of Formula (I), wherein X is C(R^{X})₂.

Another aspect includes a compound of Formula (I), wherein X is CH.

Another aspect includes a compound of Formula (I), wherein X is CD.

Another aspect includes a compound of Formula (I), wherein X is CR^{X}.

Another aspect includes a compound of Formula (I), wherein X is C(O).

Another aspect includes a compound of Formula (I), wherein X is NH.

Another aspect includes a compound of Formula (I), wherein X is O.

One aspect includes a compound of Formula (I), wherein Y is selected from the group consisting of CH₂, CD₂, CH—R^{Y}, CD-R^{Y}, C(R^{Y})₂, CH, CD, CR^{Y}, C═CH₂, C═CD₂, C═C(R^{Y})₂, N-phenyl, O, S, S(O), and SO₂.

Another aspect includes a compound of Formula (I), wherein Y is CH₂.

Another aspect includes a compound of Formula (I), wherein Y is CD₂.

Another aspect includes a compound of Formula (I), wherein Y is CH—R^{Y}.

Another aspect includes a compound of Formula (I), wherein Y is C(R^{Y})₂.

Another aspect includes a compound of Formula (I), wherein Y is CH.

Another aspect includes a compound of Formula (I), wherein Y is CD.

Another aspect includes a compound of Formula (I), wherein Y is CR^{Y}.

Another aspect includes a compound of Formula (I), wherein Y is C═CH₂.

Another aspect includes a compound of Formula (I), wherein Y is O.

Another aspect includes a compound of Formula (I), wherein Y is N—C₁₋₄alkyl, wherein C₁₋₄alkyl is selected from methyl, ethyl, propyl, isopropyl, butyl, and tert-butyl.

Another aspect includes a compound of Formula (I), wherein Y is N—CH₃.

One aspect includes a compound of Formula (I), wherein Z is selected from the group consisting of CH₂, CD₂, CH—R^{Z}, CD-R^{Z}, C(R^{Z})₂, CH, CD, CR^{Z}, NH, N—C₁₋₄alkyl, N-phenyl, O, S, S(O), and SO₂.

Another aspect includes a compound of Formula (I), wherein Z is CH₂.

Another aspect includes a compound of Formula (I), wherein Z is CD₂.

Another aspect includes a compound of Formula (I), wherein Z is CH—R^{Z}.

Another aspect includes a compound of Formula (I), wherein Z is C(R^{Z})₂.

Another aspect includes a compound of Formula (I), wherein Z is NH.

Another aspect includes a compound of Formula (I), wherein Z is N-phenyl.

Another aspect includes a compound of Formula (I), wherein Z is O.

One aspect includes a compound of Formula (I), wherein each R^{W}, R^{X}, R^{Y}, R^{Z} are independently selected from the group consisting of halo, hydroxy, C₁₋₆alkyl, halo-C₁₋₆alkyl, C₁₋₆alkoxy, halo-C₁₋₆alkoxy, amino, C₁₋₄alkyl-amino, (C₁₋₄alkyl)₂-amino, CO₂H, CO₂C₁₋₆alkyl, C(O)NH₂, C(O)N(C₁₋₆alkyl)₂, C(O)-heterocyclyl, and C(O)NH-phenyl, and wherein each R^{W}, R^{X}, R^{Y}, R^{Z} may combine to form a carbocyclic or heterocyclic ring.

Another aspect includes a compound of Formula (I), wherein each R^{W}, R^{X}, R^{Y}, R^{Z} are independently halo selected from fluoro, chloro, bromo, and iodo.

Another aspect includes a compound of Formula (I), wherein each R^{W}, R^{X}, R^{Y}, R^{Z} are independently fluoro.

Another aspect includes a compound of Formula (I), wherein each R^{W}, R^{X}, R^{Y}, R^{Z} are independently hydroxy.

Another aspect includes a compound of Formula (I), wherein each R^{W}, R^{X}, R^{Y}, R^{Z} are independently C₁₋₆alkyl.

Another aspect includes a compound of Formula (I), wherein each R^{W}, R^{X}, R^{Y}, R^{Z} are independently methyl.

Another aspect includes a compound of Formula (I), wherein each R^{W}, R^{X}, R^{Y}, R^{Z} are independently C₁₋₆alkoxy.

Another aspect includes a compound of Formula (I), wherein each R^{W}, R^{X}, R^{Y}, R^{Z} are independently methoxy.

Another aspect includes a compound of Formula (I), wherein each R^{W}, R^{X}, R^{Y}, R^{Z} are independently CO₂H.

Another aspect includes a compound of Formula (I), wherein each R^{W}, R^{X}, R^{Y}, R^{Z} are independently CO₂C₁₋₆alkyl.

Another aspect includes a compound of Formula (I), wherein each R^{W}, R^{X}, R^{Y}, R^{Z} are independently CO₂CH₂CH₃.

Another aspect includes a compound of Formula (I), wherein each R^{W}, R^{X}, R^{Y}, R^{Z} are independently C(O)NH₂.

Another aspect includes a compound of Formula (I), wherein each R^{W}, R^{X}, R^{Y}, R^{Z} are independently C(O)N(C₁₋₆alkyl)₂.

Another aspect includes a compound of Formula (I), wherein each R^{W}, R^{X}, R^{Y}, R^{Z} are independently C(O)N(CH₃)₂.

Another aspect includes a compound of Formula (I), wherein each R^{W}, R^{X}, R^{Y}, R^{Z} are independently C(O)-heterocyclyl.

Another aspect includes a compound of Formula (I), wherein each R^{W}, R^{X}, R^{Y}, R^{Z} are independently

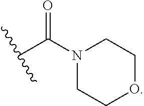 [SMILES: *C(=O)N1CCOCC1]

Another aspect includes a compound of Formula (I), wherein each R^{W}, R^{X}, R^{Y}, R^{Z} are independently C(O)NH-phenyl.

Another aspect includes a compound of Formula (I), wherein each R^{W}, R^{X}, R^{Y}, R^{Z} are independently

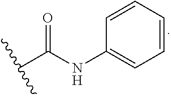 [SMILES: *C(=O)Nc1ccccc1]

One aspect includes a compound of Formula (I), wherein n is selected from the group consisting of 0, 1, 2, and 3.

Another aspect includes a compound of Formula (I), wherein n is 0.

Another aspect includes a compound of Formula (I), wherein n is 1.

Another aspect includes a compound of Formula (I), wherein n is 2.

Another aspect includes a compound of Formula (I), wherein n is 3.

One aspect includes a compound of Formula (I), wherein [symbol] independently represents a single bond or a double bond as valency permits.

Another aspect includes a compound of Formula (I), wherein [symbol] represents a single bond as valency permits.

Another aspect includes a compound of Formula (I), wherein [symbol] represents a double bond as valency permits.

One aspect includes a compound of Formula (I), wherein Ring Q is selected from the group consisting of

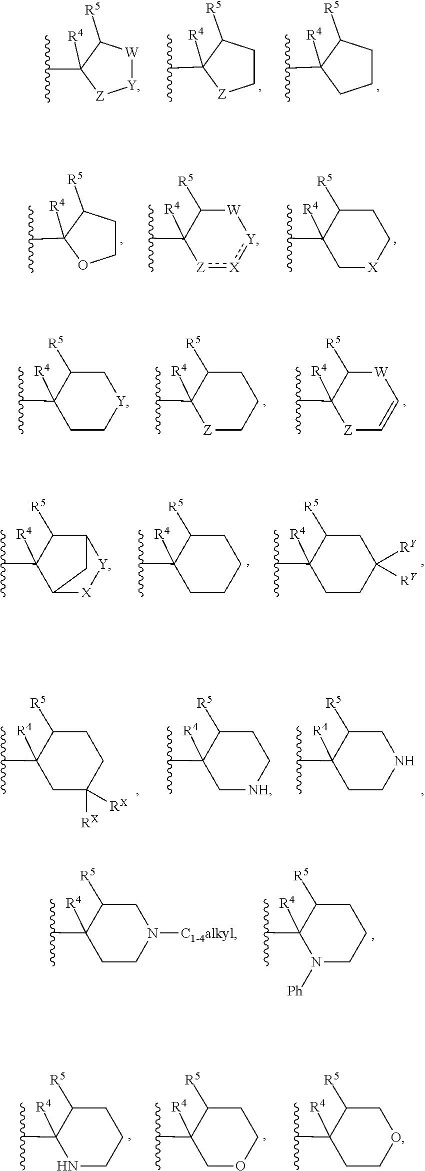

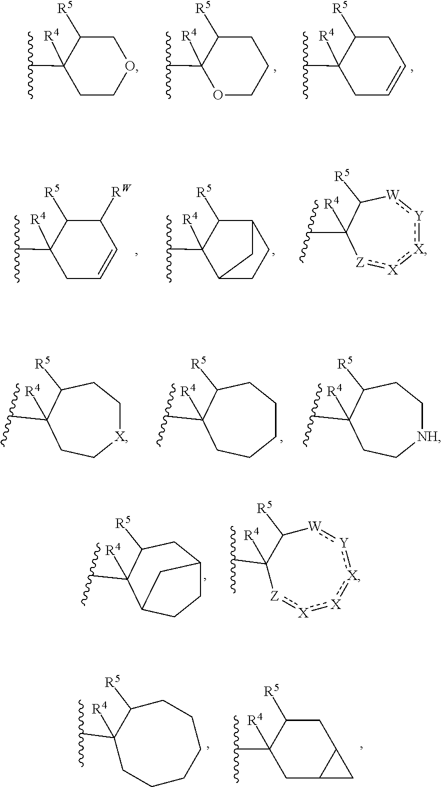

and any stereoisomers thereof.

One aspect includes a compound of Formula (I), wherein Ring Q is selected from the group consisting of

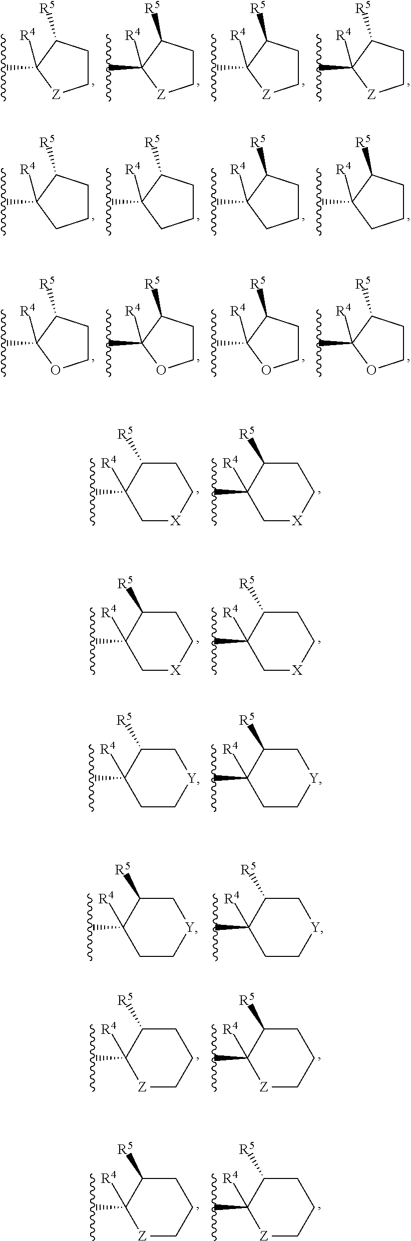

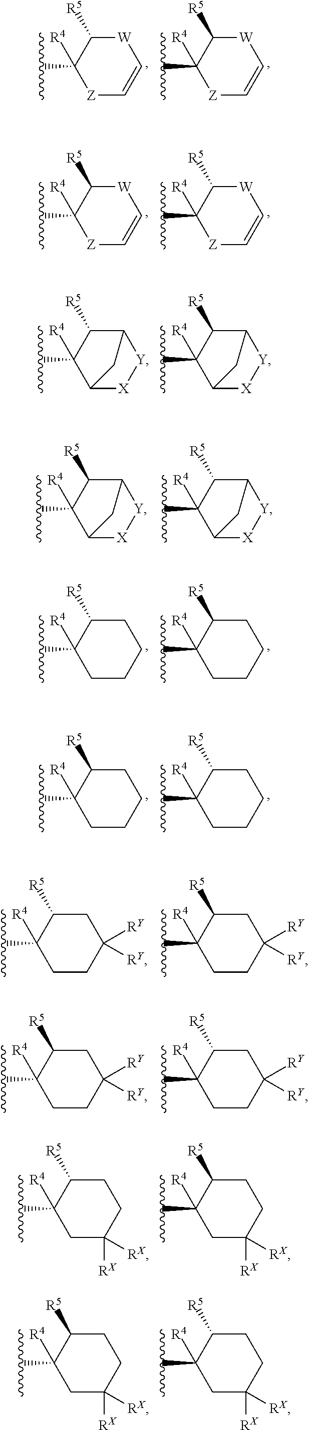

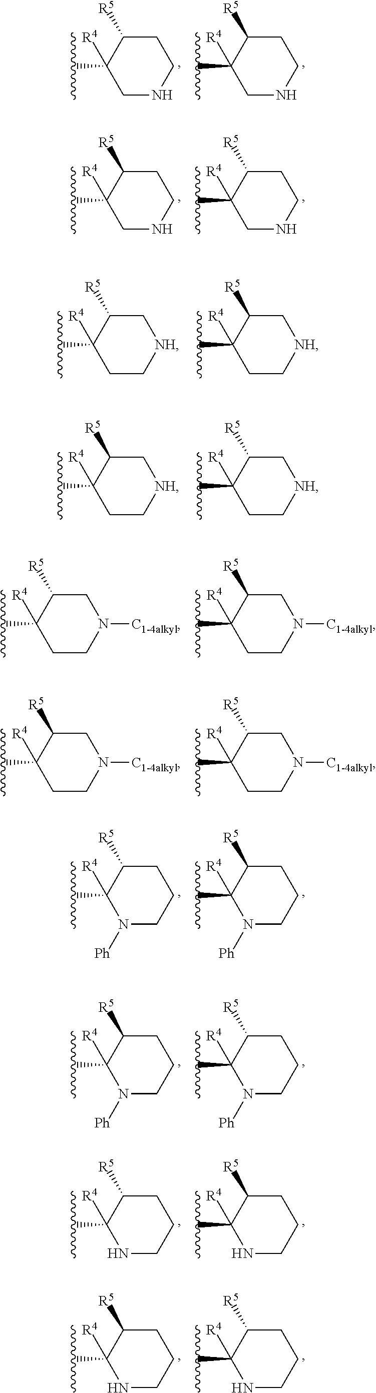

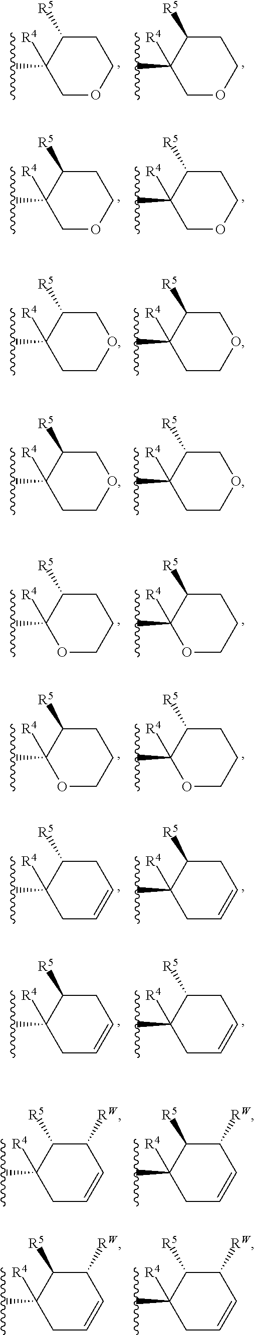

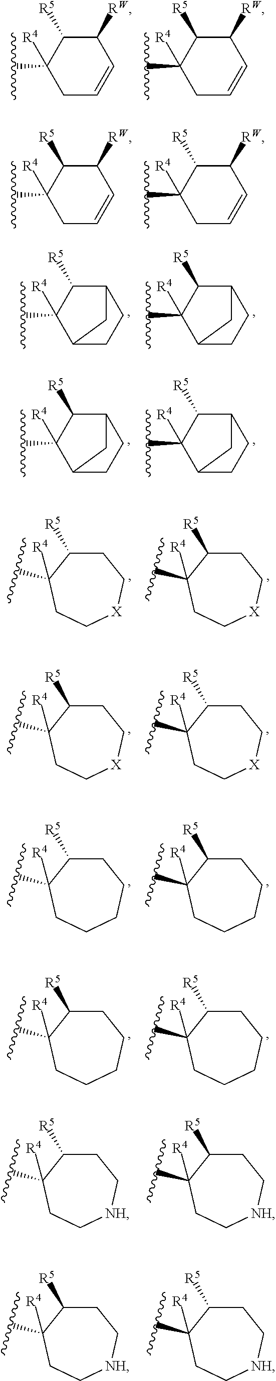

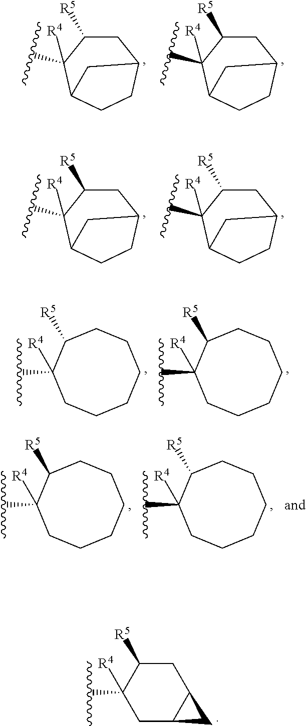

One aspect includes a compound of Formula (I), wherein Ring Q is selected from the group consisting of

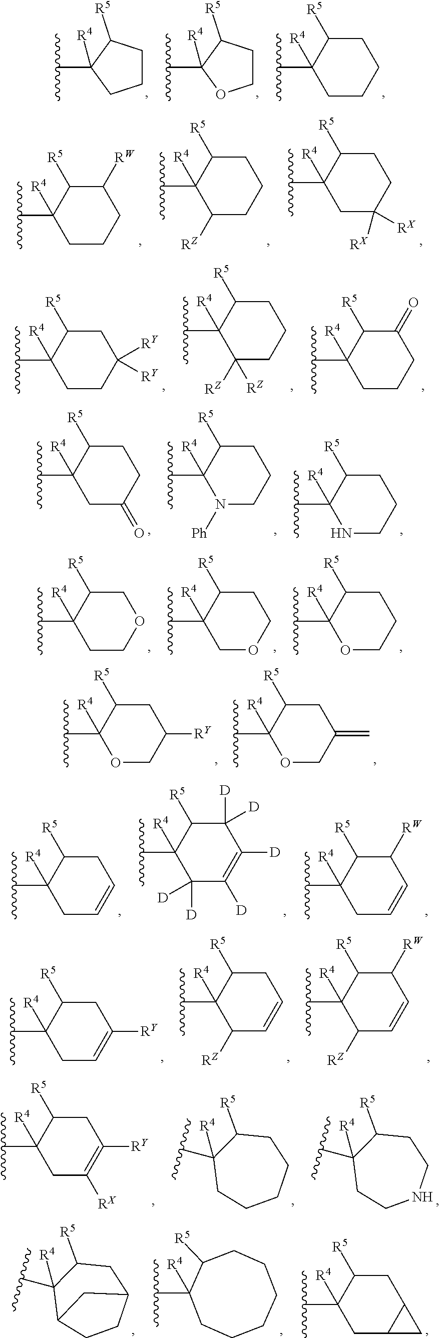

and any stereoisomers thereof.

One aspect includes a compound of Formula (I), wherein Ring Q is selected from the group consisting of

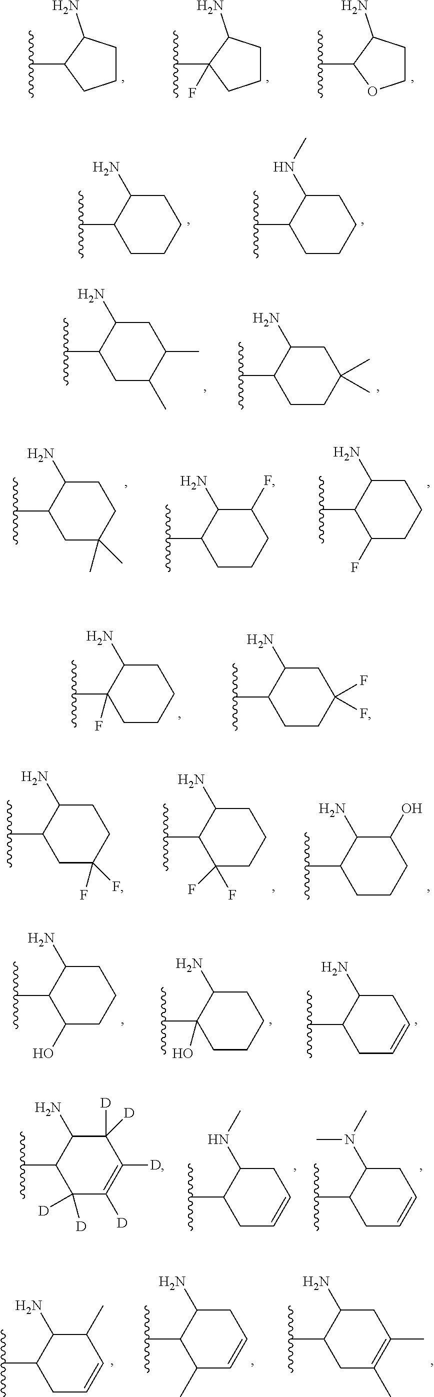

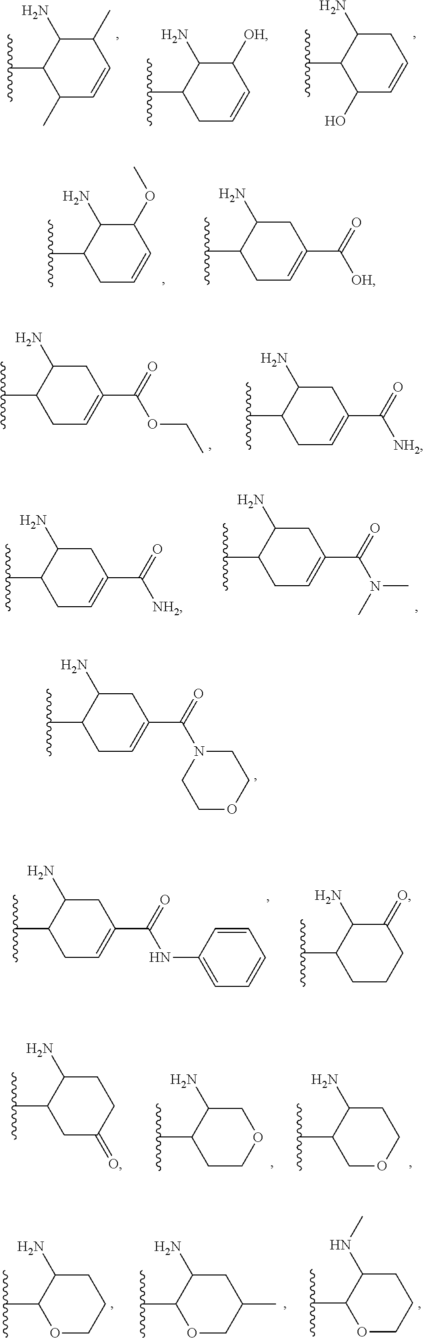

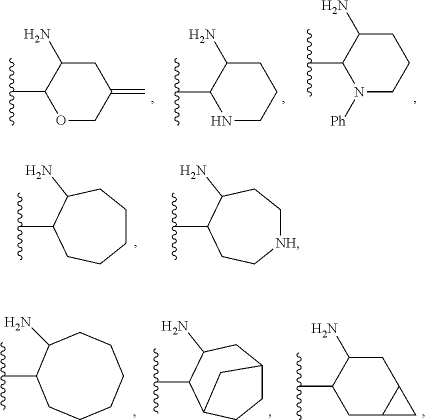

and any stereoisomers thereof.

One aspect includes a compound of Formula (I), wherein Ring Q is selected from the group consisting of

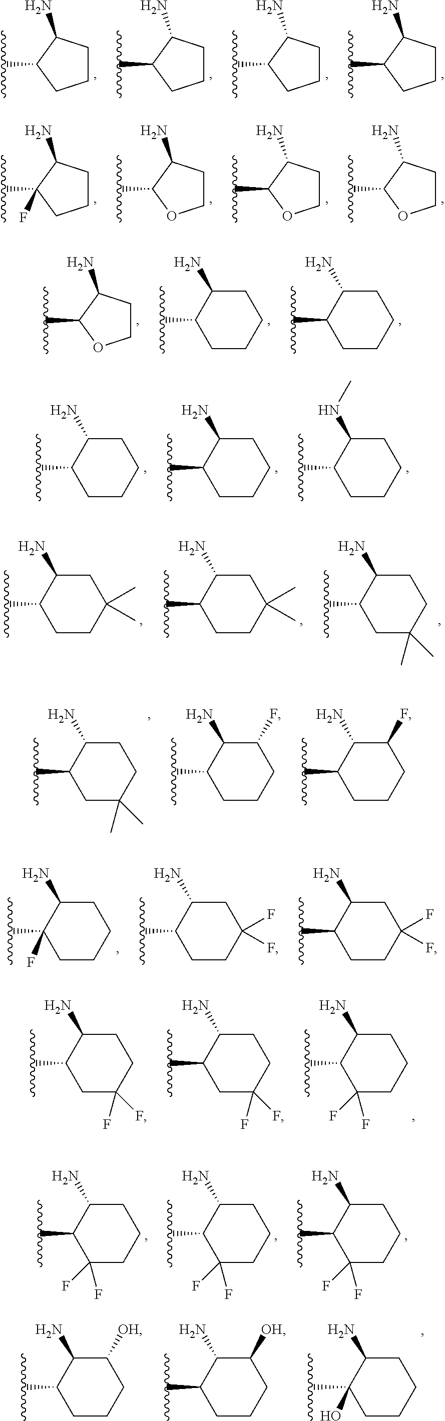

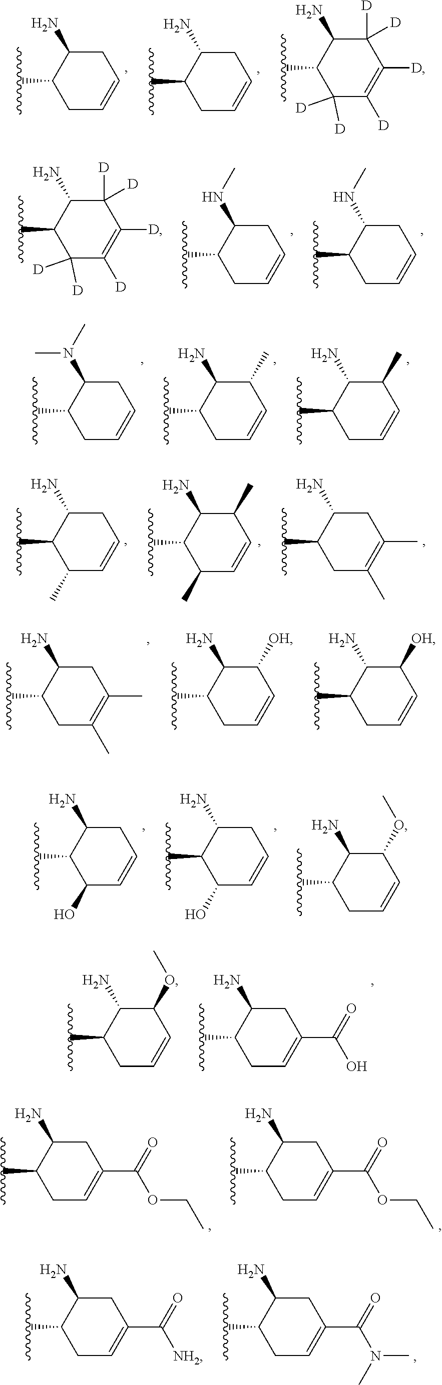

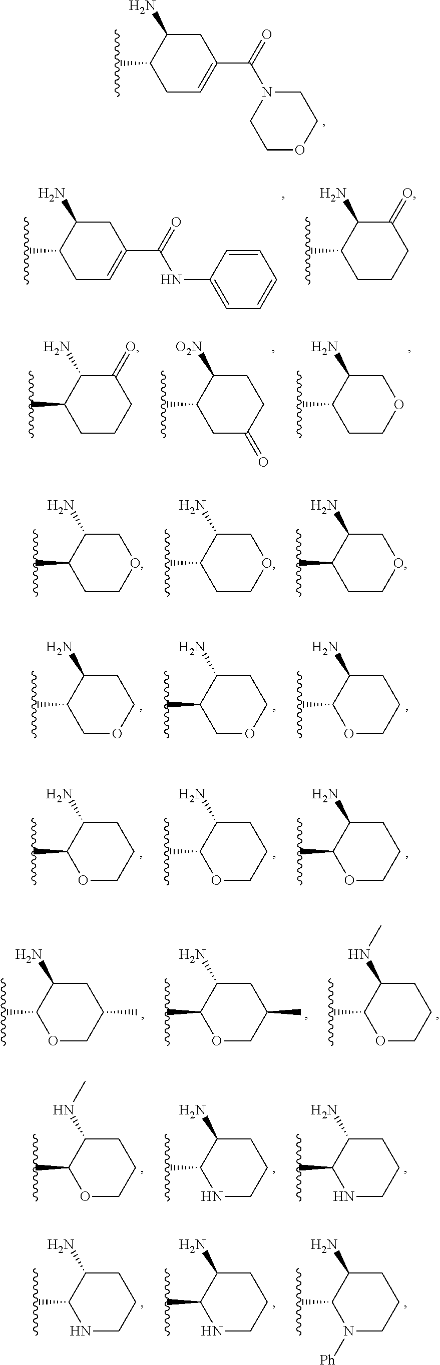

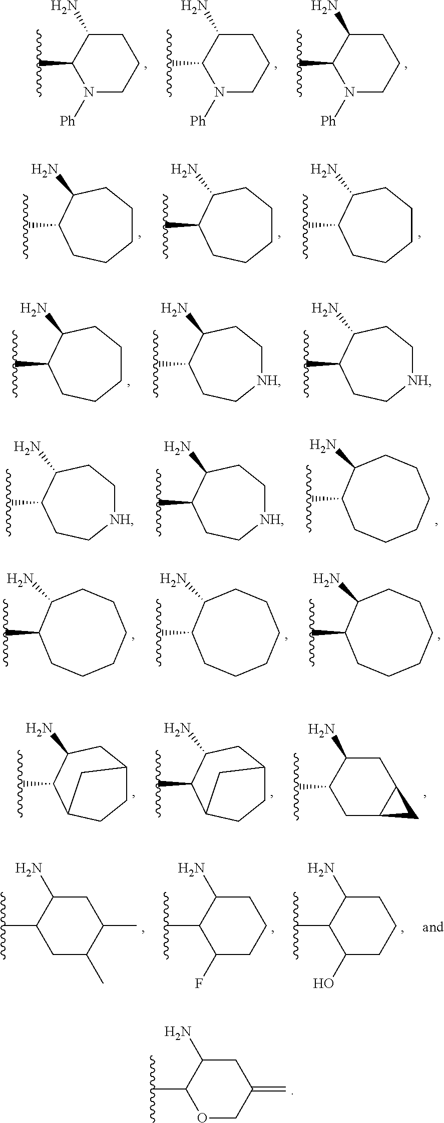

One aspect includes a compound of Formula (I) or a form thereof selected from the group consisting of the following, wherein “^{#}” indicates that the compound is a racemic mixture of enantiomers, and wherein “^{&}” indicates that the compound may exist as the opposite enantiomer:

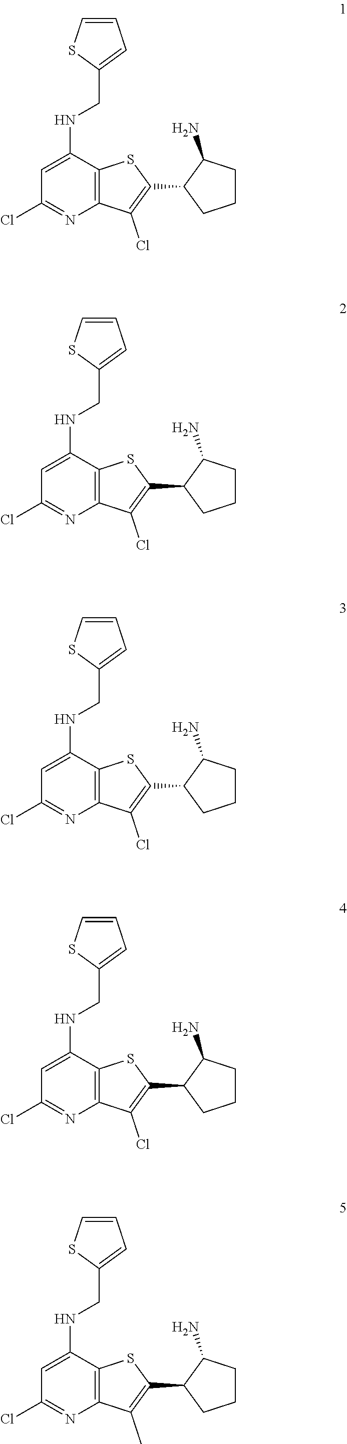 [SMILES: N[C@H]1CCC[C@@H]1c1sc2c(NCc3cccs3)cc(Cl)nc2c1Cl ; N[C@@H]1CCC[C@H]1c1sc2c(NCc3cccs3)cc(Cl)nc2c1Cl ; N[C@@H]1CCC[C@@H]1c1sc2c(NCc3cccs3)cc(Cl)nc2c1Cl ; N[C@H]1CCC[C@H]1c1sc2c(NCc3cccs3)cc(Cl)nc2c1Cl ; Cc1c([C@@H]2CCC[C@H]2N)sc2c(NCc3cccs3)cc(Cl)nc12]

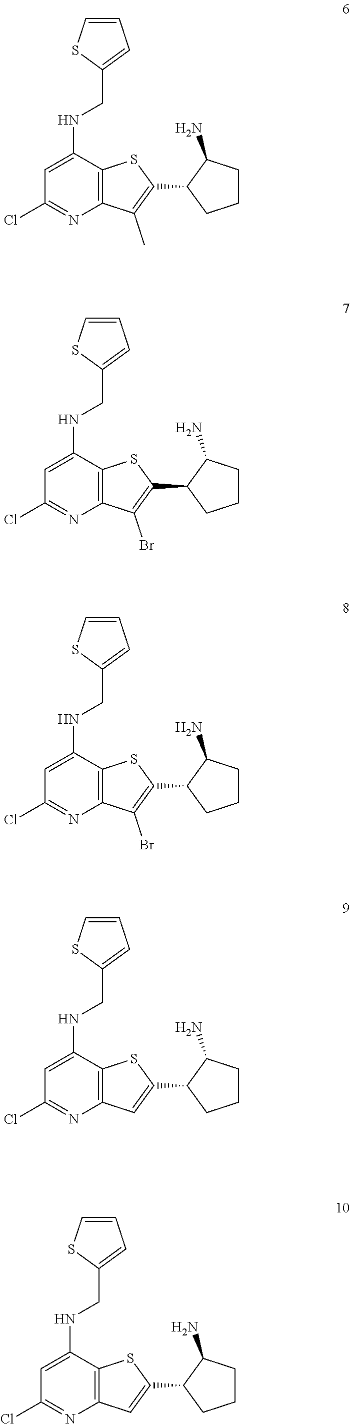 [SMILES: Cc1c([C@H]2CCC[C@@H]2N)sc2c(NCc3cccs3)cc(Cl)nc12 ; N[C@@H]1CCC[C@H]1c1sc2c(NCc3cccs3)cc(Cl)nc2c1Br ; N[C@H]1CCC[C@@H]1c1sc2c(NCc3cccs3)cc(Cl)nc2c1Br ; N[C@@H]1CCC[C@@H]1c1cc2nc(Cl)cc(NCc3cccs3)c2s1 ; N[C@H]1CCC[C@@H]1c1cc2nc(Cl)cc(NCc3cccs3)c2s1]

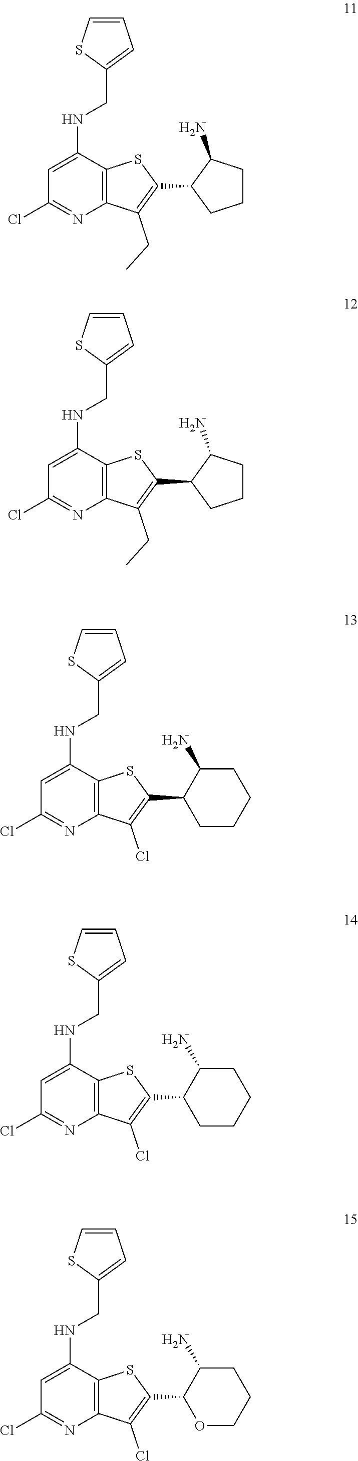

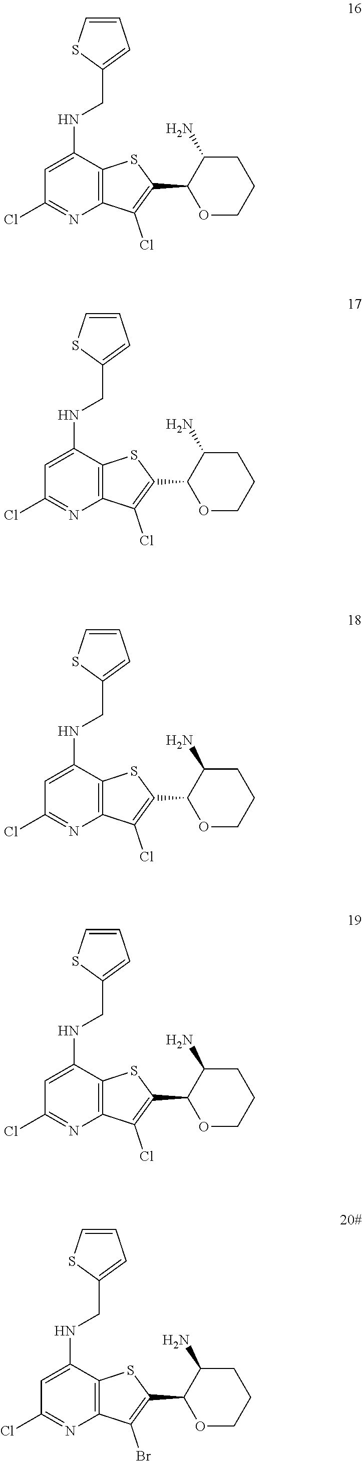 [SMILES: N[C@@H]1CCCO[C@H]1c1sc2c(NCc3cccs3)cc(Cl)nc2c1Cl ; N[C@@H]1CCCO[C@@H]1c1sc2c(NCc3cccs3)cc(Cl)nc2c1Cl ; N[C@H]1CCCO[C@@H]1c1sc2c(NCc3cccs3)cc(Cl)nc2c1Cl ; N[C@H]1CCCO[C@H]1c1sc2c(NCc3cccs3)cc(Cl)nc2c1Cl ; N[C@H]1CCCO[C@H]1c1sc2c(NCc3cccs3)cc(Cl)nc2c1Br]

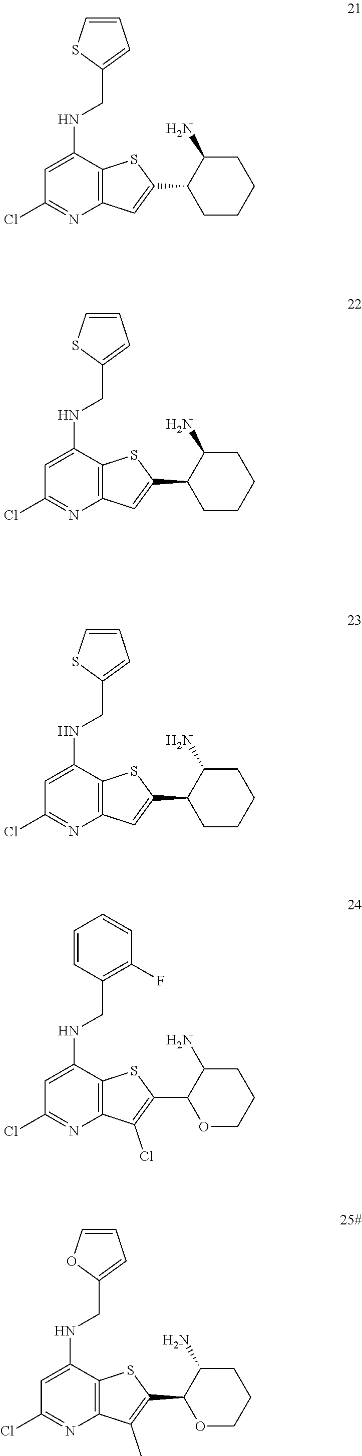 [SMILES: N[C@H]1CCCC[C@@H]1c1cc2nc(Cl)cc(NCc3cccs3)c2s1 ; N[C@H]1CCCC[C@H]1c1cc2nc(Cl)cc(NCc3cccs3)c2s1 ; N[C@@H]1CCCC[C@H]1c1cc2nc(Cl)cc(NCc3cccs3)c2s1 ; NC1CCCOC1c1sc2c(NCc3ccccc3F)cc(Cl)nc2c1Cl ; Cc1c([C@@H]2OCCC[C@H]2N)sc2c(NCc3ccco3)cc(Cl)nc12]

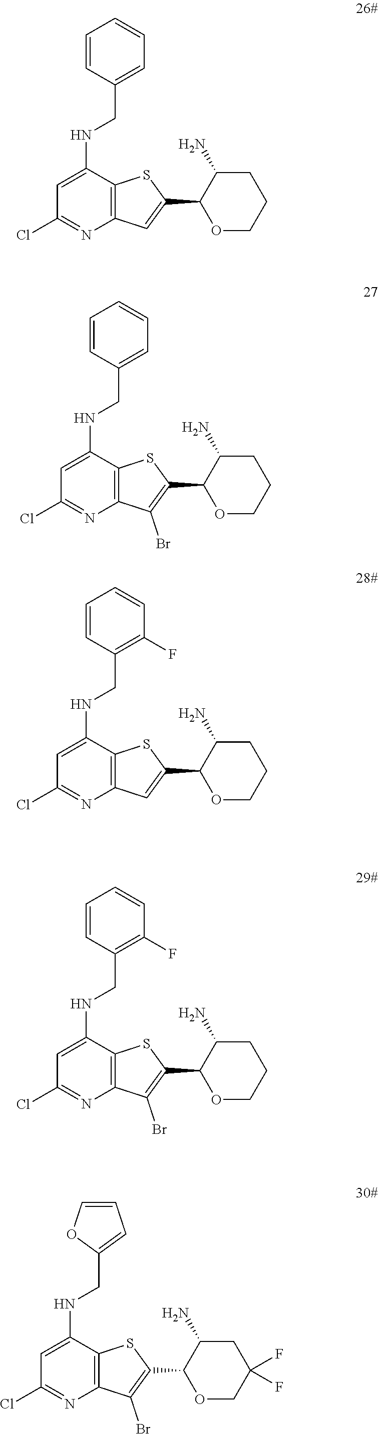 [SMILES: N[C@@H]1CCCO[C@H]1c1cc2nc(Cl)cc(NCc3ccccc3)c2s1 ; N[C@@H]1CCCO[C@H]1c1sc2c(NCc3ccccc3)cc(Cl)nc2c1Br ; N[C@@H]1CCCO[C@H]1c1cc2nc(Cl)cc(NCc3ccccc3F)c2s1 ; N[C@@H]1CCCO[C@H]1c1sc2c(NCc3ccccc3F)cc(Cl)nc2c1Br ; N[C@@H]1CC(F)(F)CO[C@@H]1c1sc2c(NCc3ccco3)cc(Cl)nc2c1Br]

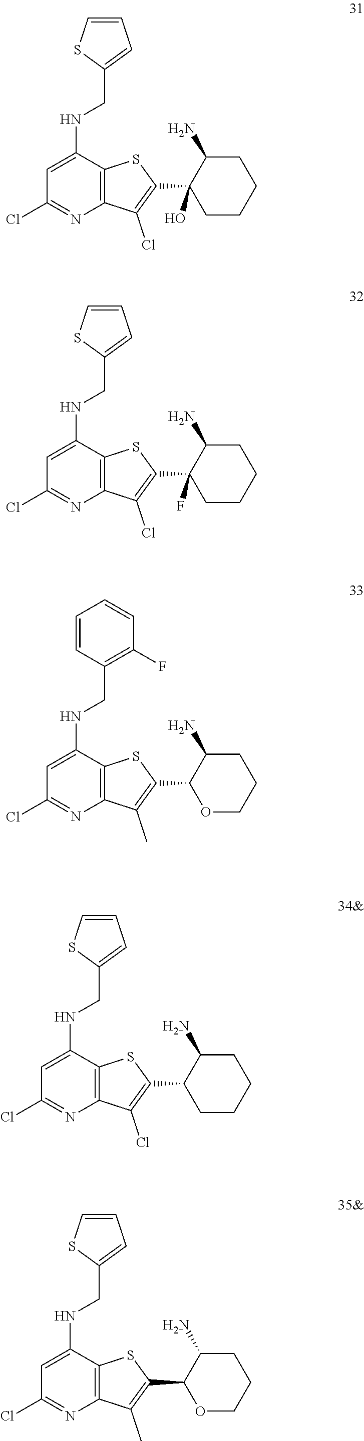 [SMILES: N[C@H]1CCCC[C@]1(O)c1sc2c(NCc3cccs3)cc(Cl)nc2c1Cl ; N[C@H]1CCCC[C@]1(F)c1sc2c(NCc3cccs3)cc(Cl)nc2c1Cl ; Cc1c([C@H]2OCCC[C@@H]2N)sc2c(NCc3ccccc3F)cc(Cl)nc12 ; N[C@H]1CCCC[C@@H]1c1sc2c(NCc3cccs3)cc(Cl)nc2c1Cl ; Cc1c([C@@H]2OCCC[C@H]2N)sc2c(NCc3cccs3)cc(Cl)nc12]

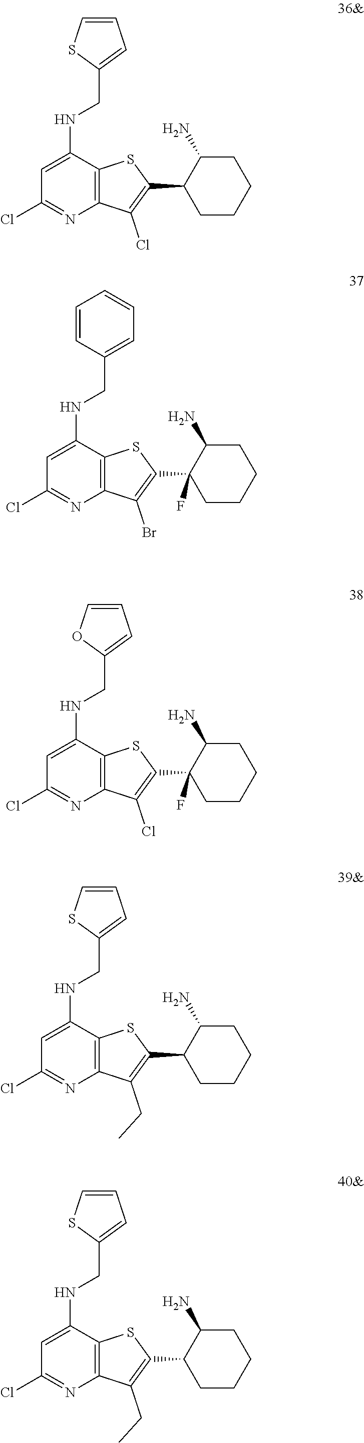 [SMILES: N[C@@H]1CCCC[C@H]1c1sc2c(NCc3cccs3)cc(Cl)nc2c1Cl ; N[C@H]1CCCC[C@]1(F)c1sc2c(NCc3ccccc3)cc(Cl)nc2c1Br ; N[C@H]1CCCC[C@]1(F)c1sc2c(NCc3ccco3)cc(Cl)nc2c1Cl ; CCc1c([C@@H]2CCCC[C@H]2N)sc2c(NCc3cccs3)cc(Cl)nc12 ; CCc1c([C@H]2CCCC[C@@H]2N)sc2c(NCc3cccs3)cc(Cl)nc12]

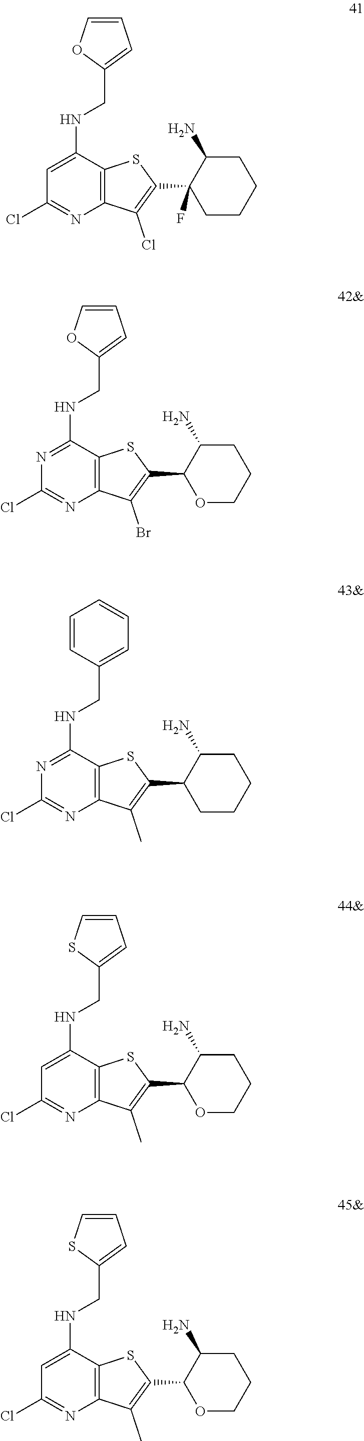 [SMILES: N[C@H]1CCCC[C@]1(F)c1sc2c(NCc3ccco3)cc(Cl)nc2c1Cl ; N[C@@H]1CCCO[C@H]1c1sc2c(NCc3ccco3)nc(Cl)nc2c1Br ; Cc1c([C@@H]2CCCC[C@H]2N)sc2c(NCc3ccccc3)nc(Cl)nc12 ; Cc1c([C@@H]2OCCC[C@H]2N)sc2c(NCc3cccs3)cc(Cl)nc12 ; Cc1c([C@H]2OCCC[C@@H]2N)sc2c(NCc3cccs3)cc(Cl)nc12]

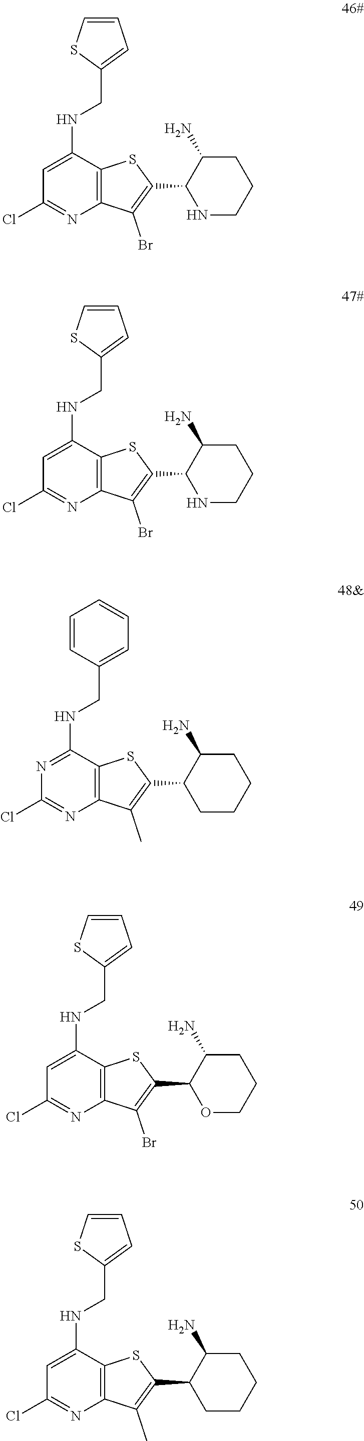 [SMILES: N[C@@H]1CCCN[C@@H]1c1sc2c(NCc3cccs3)cc(Cl)nc2c1Br ; N[C@H]1CCCN[C@@H]1c1sc2c(NCc3cccs3)cc(Cl)nc2c1Br ; Cc1c([C@H]2CCCC[C@@H]2N)sc2c(NCc3ccccc3)nc(Cl)nc12 ; N[C@@H]1CCCO[C@H]1c1sc2c(NCc3cccs3)cc(Cl)nc2c1Br ; Cc1c([C@@H]2CCCC[C@@H]2N)sc2c(NCc3cccs3)cc(Cl)nc12]

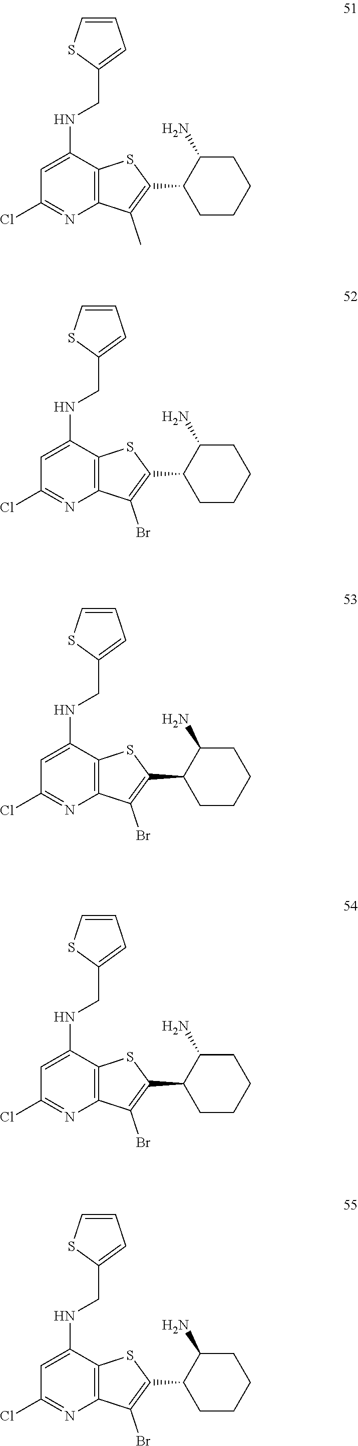 [SMILES: Cc1c([C@H]2CCCC[C@H]2N)sc2c(NCc3cccs3)cc(Cl)nc12 ; N[C@@H]1CCCC[C@@H]1c1sc2c(NCc3cccs3)cc(Cl)nc2c1Br ; N[C@H]1CCCC[C@H]1c1sc2c(NCc3cccs3)cc(Cl)nc2c1Br ; N[C@@H]1CCCC[C@H]1c1sc2c(NCc3cccs3)cc(Cl)nc2c1Br ; N[C@H]1CCCC[C@@H]1c1sc2c(NCc3cccs3)cc(Cl)nc2c1Br]

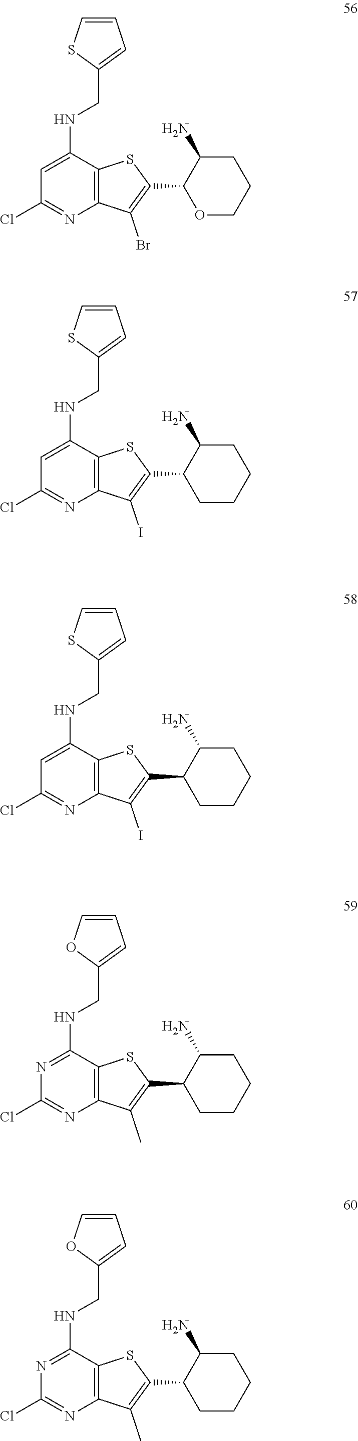 [SMILES: N[C@H]1CCCO[C@@H]1c1sc2c(NCc3cccs3)cc(Cl)nc2c1Br ; N[C@H]1CCCC[C@@H]1c1sc2c(NCc3cccs3)cc(Cl)nc2c1I ; N[C@@H]1CCCC[C@H]1c1sc2c(NCc3cccs3)cc(Cl)nc2c1I ; Cc1c([C@@H]2CCCC[C@H]2N)sc2c(NCc3ccco3)nc(Cl)nc12 ; Cc1c([C@H]2CCCC[C@@H]2N)sc2c(NCc3ccco3)nc(Cl)nc12]

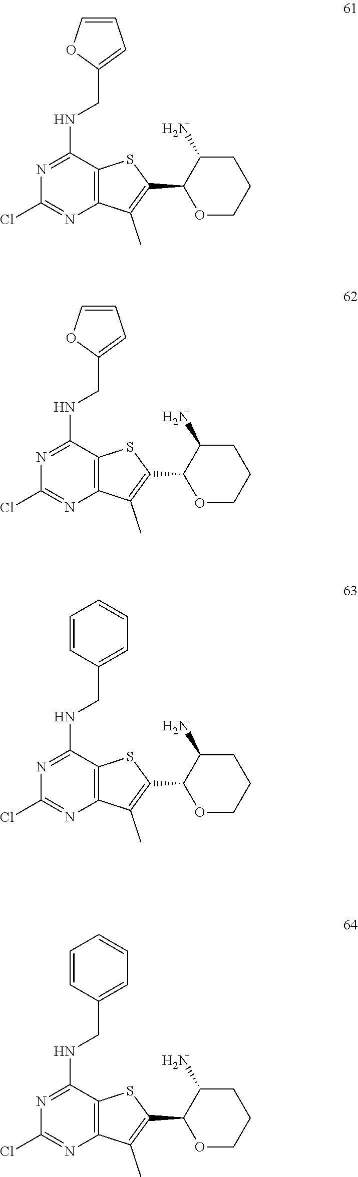 [SMILES: Cc1c([C@@H]2OCCC[C@H]2N)sc2c(NCc3ccco3)nc(Cl)nc12 ; Cc1c([C@H]2OCCC[C@@H]2N)sc2c(NCc3ccco3)nc(Cl)nc12 ; Cc1c([C@H]2OCCC[C@@H]2N)sc2c(NCc3ccccc3)nc(Cl)nc12 ; Cc1c([C@@H]2OCCC[C@H]2N)sc2c(NCc3ccccc3)nc(Cl)nc12]

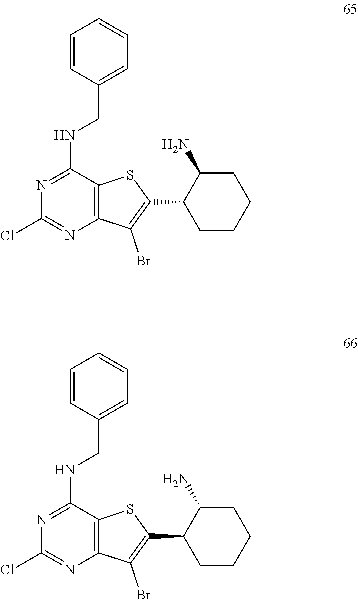 [SMILES: N[C@H]1CCCC[C@@H]1c1sc2c(NCc3ccccc3)nc(Cl)nc2c1Br ; N[C@@H]1CCCC[C@H]1c1sc2c(NCc3ccccc3)nc(Cl)nc2c1Br]

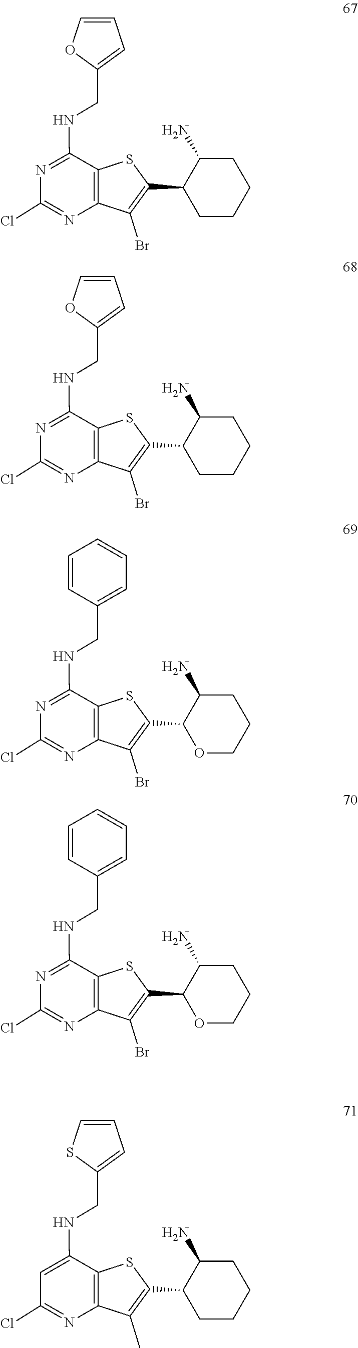

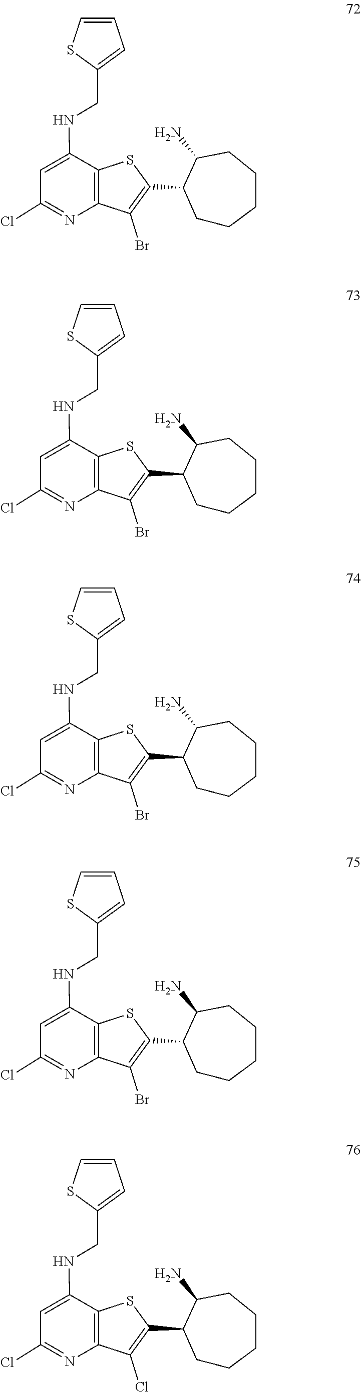 [SMILES: N[C@@H]1CCCCC[C@@H]1c1sc2c(NCc3cccs3)cc(Cl)nc2c1Br ; N[C@H]1CCCCC[C@H]1c1sc2c(NCc3cccs3)cc(Cl)nc2c1Br ; N[C@@H]1CCCCC[C@H]1c1sc2c(NCc3cccs3)cc(Cl)nc2c1Br ; N[C@H]1CCCCC[C@@H]1c1sc2c(NCc3cccs3)cc(Cl)nc2c1Br ; N[C@H]1CCCCC[C@H]1c1sc2c(NCc3cccs3)cc(Cl)nc2c1Cl]

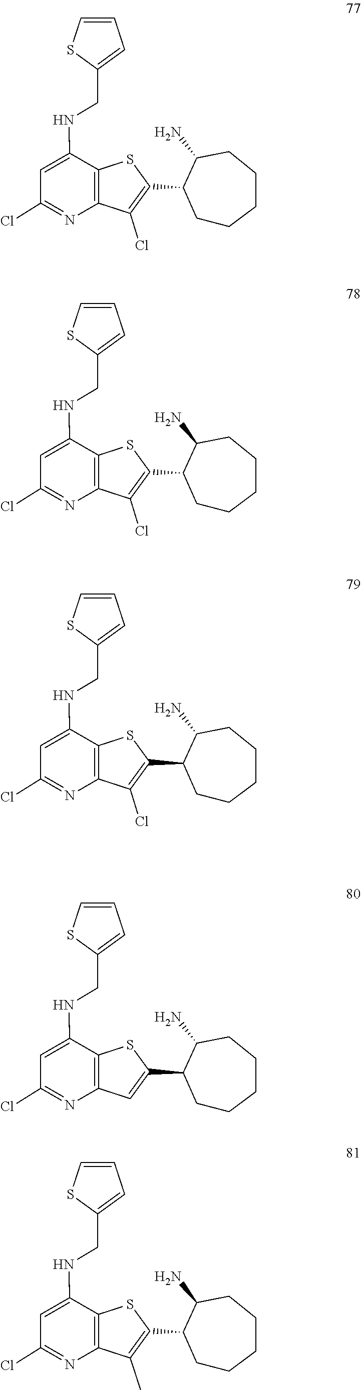 [SMILES: N[C@@H]1CCCCC[C@@H]1c1sc2c(NCc3cccs3)cc(Cl)nc2c1Cl ; N[C@H]1CCCCC[C@@H]1c1sc2c(NCc3cccs3)cc(Cl)nc2c1Cl ; N[C@@H]1CCCCC[C@H]1c1sc2c(NCc3cccs3)cc(Cl)nc2c1Cl ; N[C@@H]1CCCCC[C@H]1c1cc2nc(Cl)cc(NCc3cccs3)c2s1 ; Cc1c([C@H]2CCCCC[C@@H]2N)sc2c(NCc3cccs3)cc(Cl)nc12]

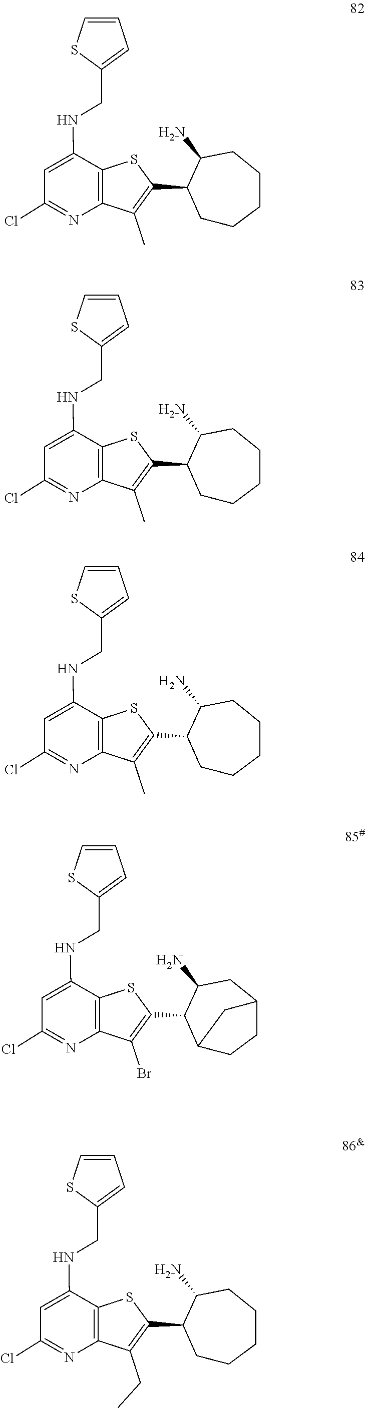 [SMILES: Cc1c([C@@H]2CCCCC[C@@H]2N)sc2c(NCc3cccs3)cc(Cl)nc12 ; Cc1c([C@@H]2CCCCC[C@H]2N)sc2c(NCc3cccs3)cc(Cl)nc12 ; Cc1c([C@H]2CCCCC[C@H]2N)sc2c(NCc3cccs3)cc(Cl)nc12 ; N[C@H]1CC2CCC(C2)[C@@H]1c1sc2c(NCc3cccs3)cc(Cl)nc2c1Br ; CCc1c([C@@H]2CCCCC[C@H]2N)sc2c(NCc3cccs3)cc(Cl)nc12]

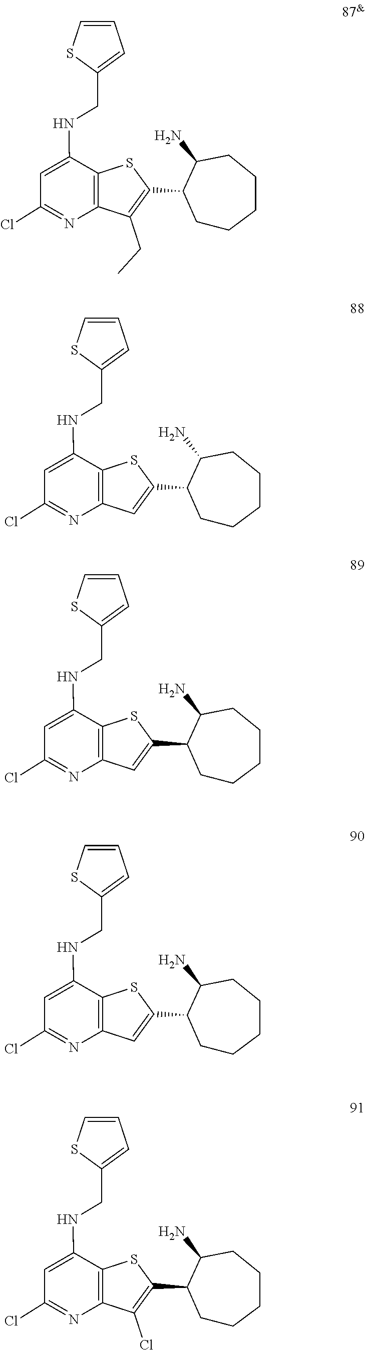

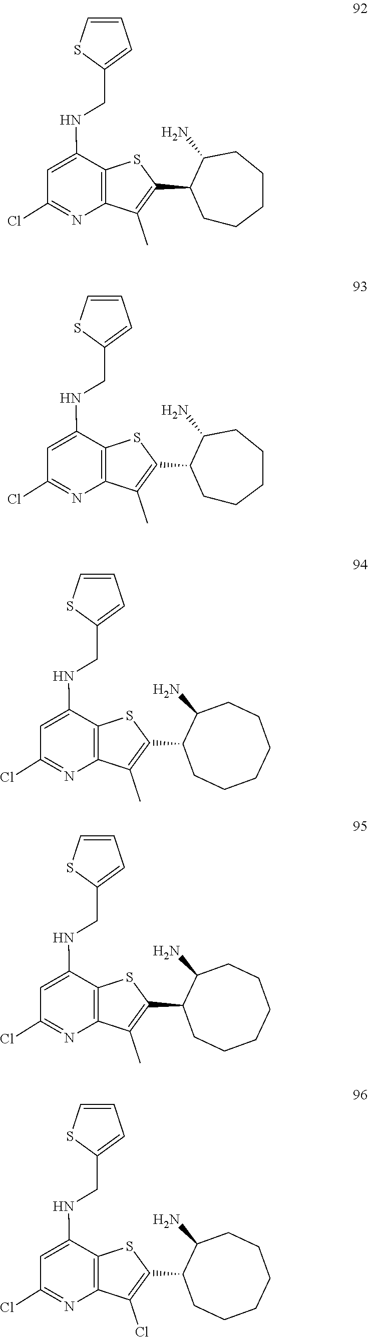

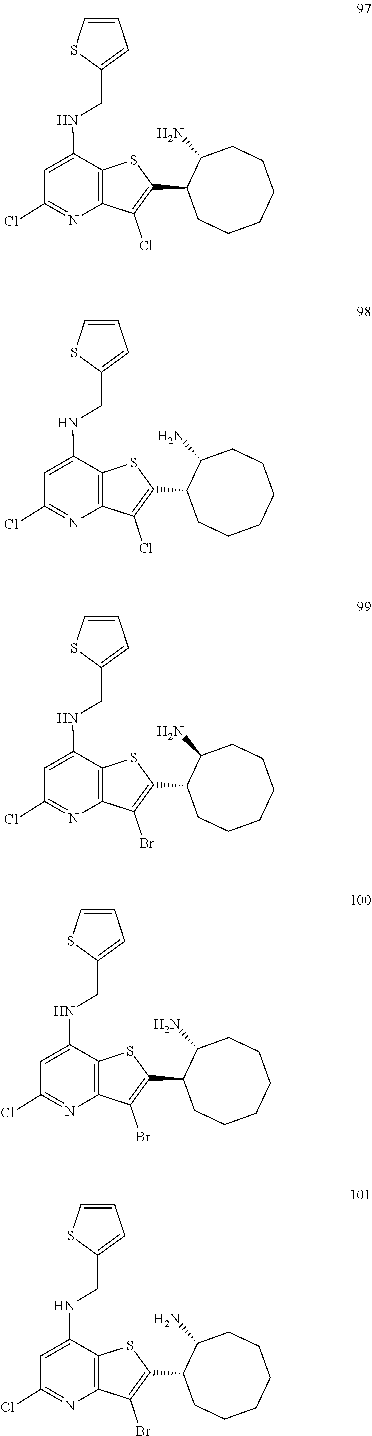 [SMILES: N[C@@H]1CCCCCC[C@H]1c1sc2c(NCc3cccs3)cc(Cl)nc2c1Cl ; N[C@@H]1CCCCCC[C@@H]1c1sc2c(NCc3cccs3)cc(Cl)nc2c1Cl ; N[C@H]1CCCCCC[C@@H]1c1sc2c(NCc3cccs3)cc(Cl)nc2c1Br ; N[C@@H]1CCCCCC[C@H]1c1sc2c(NCc3cccs3)cc(Cl)nc2c1Br ; N[C@@H]1CCCCCC[C@@H]1c1sc2c(NCc3cccs3)cc(Cl)nc2c1Br]

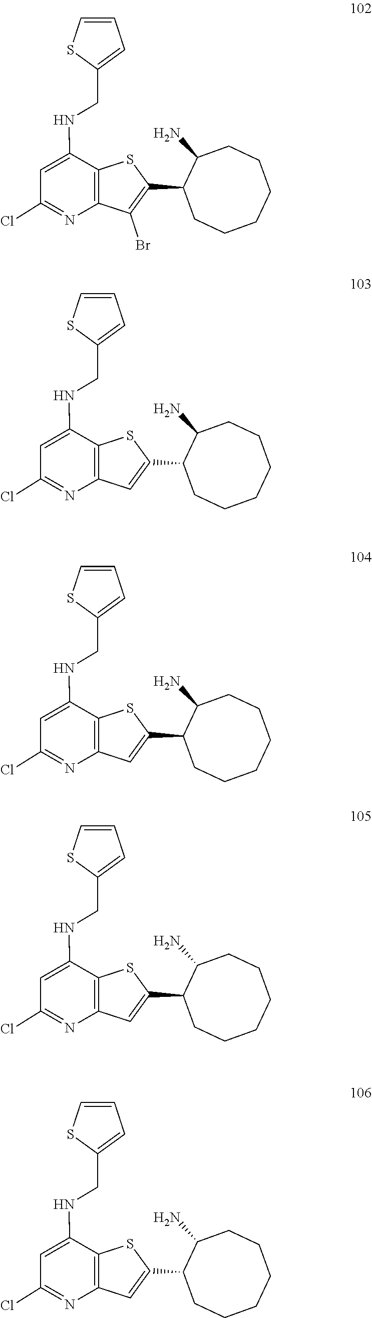 [SMILES: N[C@H]1CCCCCC[C@H]1c1sc2c(NCc3cccs3)cc(Cl)nc2c1Br ; N[C@H]1CCCCCC[C@@H]1c1cc2nc(Cl)cc(NCc3cccs3)c2s1 ; N[C@H]1CCCCCC[C@H]1c1cc2nc(Cl)cc(NCc3cccs3)c2s1 ; N[C@@H]1CCCCCC[C@H]1c1cc2nc(Cl)cc(NCc3cccs3)c2s1 ; N[C@@H]1CCCCCC[C@@H]1c1cc2nc(Cl)cc(NCc3cccs3)c2s1]

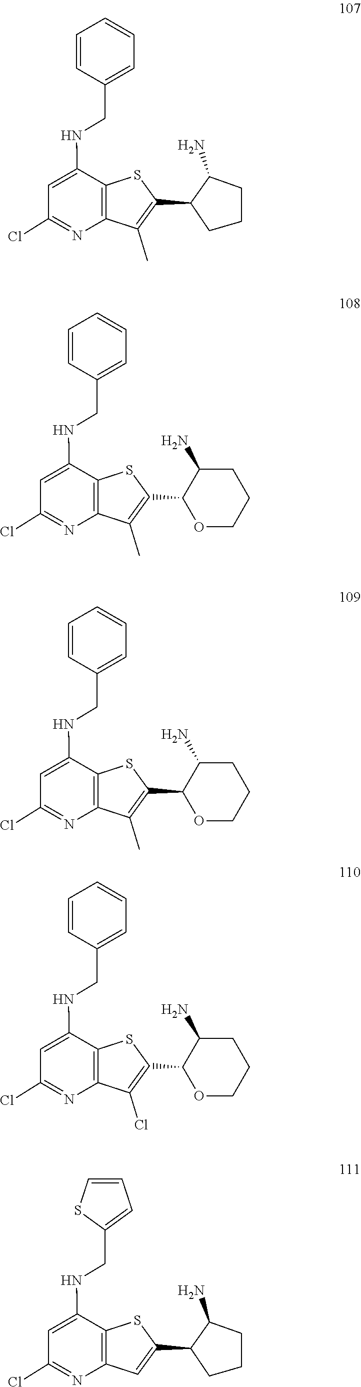 [SMILES: Cc1c([C@@H]2CCC[C@H]2N)sc2c(NCc3ccccc3)cc(Cl)nc12 ; Cc1c([C@H]2OCCC[C@@H]2N)sc2c(NCc3ccccc3)cc(Cl)nc12 ; Cc1c([C@@H]2OCCC[C@H]2N)sc2c(NCc3ccccc3)cc(Cl)nc12 ; N[C@H]1CCCO[C@@H]1c1sc2c(NCc3ccccc3)cc(Cl)nc2c1Cl ; N[C@H]1CCC[C@H]1c1cc2nc(Cl)cc(NCc3cccs3)c2s1]

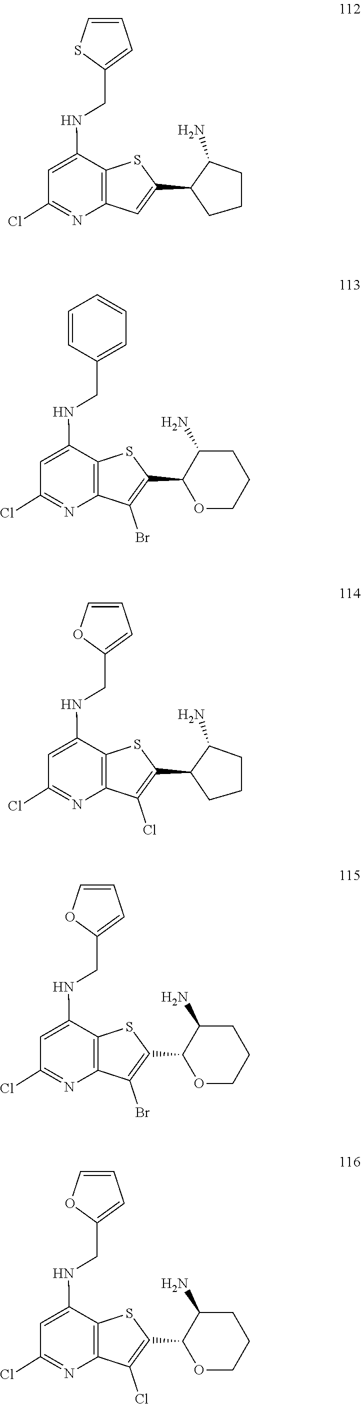 [SMILES: N[C@@H]1CCC[C@H]1c1cc2nc(Cl)cc(NCc3cccs3)c2s1 ; N[C@@H]1CCCO[C@H]1c1sc2c(NCc3ccccc3)cc(Cl)nc2c1Br ; N[C@@H]1CCC[C@H]1c1sc2c(NCc3ccco3)cc(Cl)nc2c1Cl ; N[C@H]1CCCO[C@@H]1c1sc2c(NCc3ccco3)cc(Cl)nc2c1Br ; N[C@H]1CCCO[C@@H]1c1sc2c(NCc3ccco3)cc(Cl)nc2c1Cl]

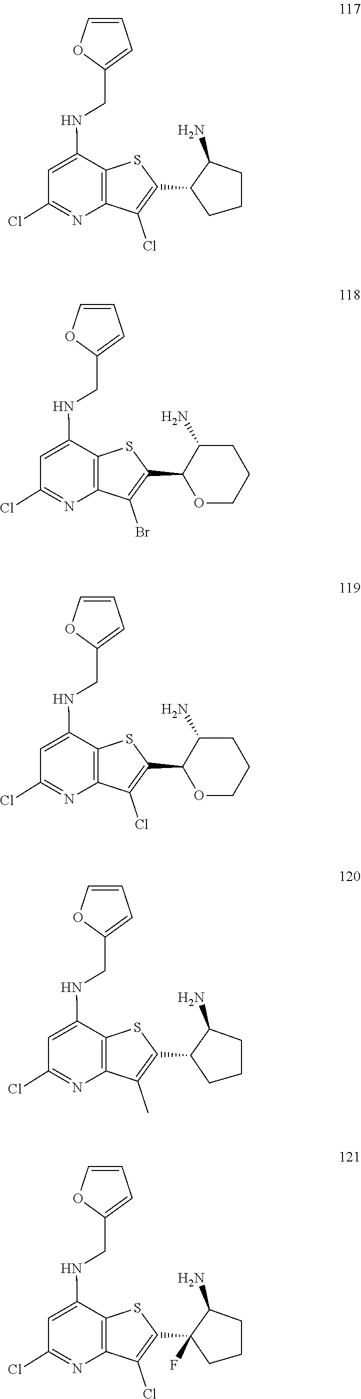 [SMILES: N[C@H]1CCC[C@@H]1c1sc2c(NCc3ccco3)cc(Cl)nc2c1Cl ; N[C@@H]1CCCO[C@H]1c1sc2c(NCc3ccco3)cc(Cl)nc2c1Br ; N[C@@H]1CCCO[C@H]1c1sc2c(NCc3ccco3)cc(Cl)nc2c1Cl ; Cc1c([C@H]2CCC[C@@H]2N)sc2c(NCc3ccco3)cc(Cl)nc12 ; N[C@H]1CCC[C@]1(F)c1sc2c(NCc3ccco3)cc(Cl)nc2c1Cl]

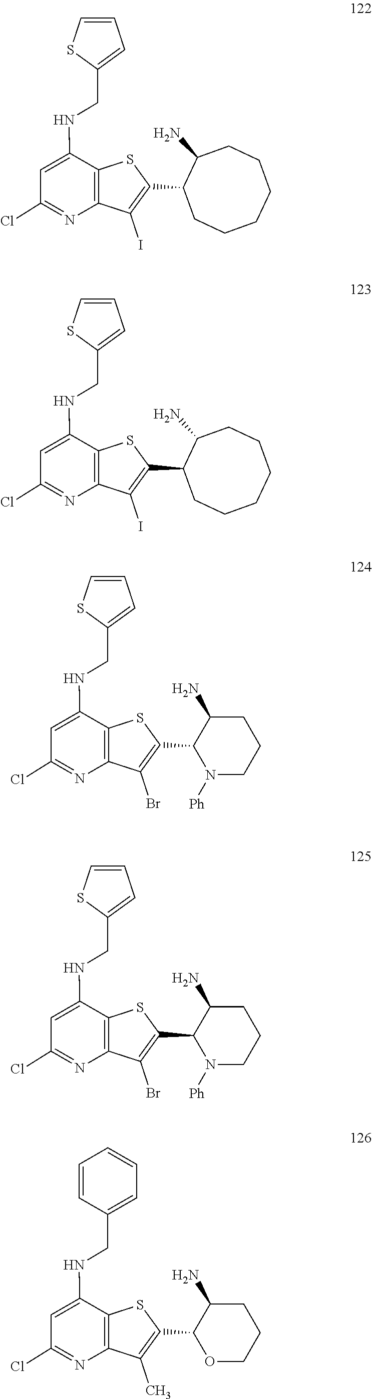 [SMILES: N[C@H]1CCCCCC[C@@H]1c1sc2c(NCc3cccs3)cc(Cl)nc2c1I ; N[C@@H]1CCCCCC[C@H]1c1sc2c(NCc3cccs3)cc(Cl)nc2c1I ; N[C@H]1CCCN(c2ccccc2)[C@@H]1c1sc2c(NCc3cccs3)cc(Cl)nc2c1Br ; N[C@H]1CCCN(c2ccccc2)[C@H]1c1sc2c(NCc3cccs3)cc(Cl)nc2c1Br ; Cc1c([C@H]2OCCC[C@@H]2N)sc2c(NCc3ccccc3)cc(Cl)nc12]

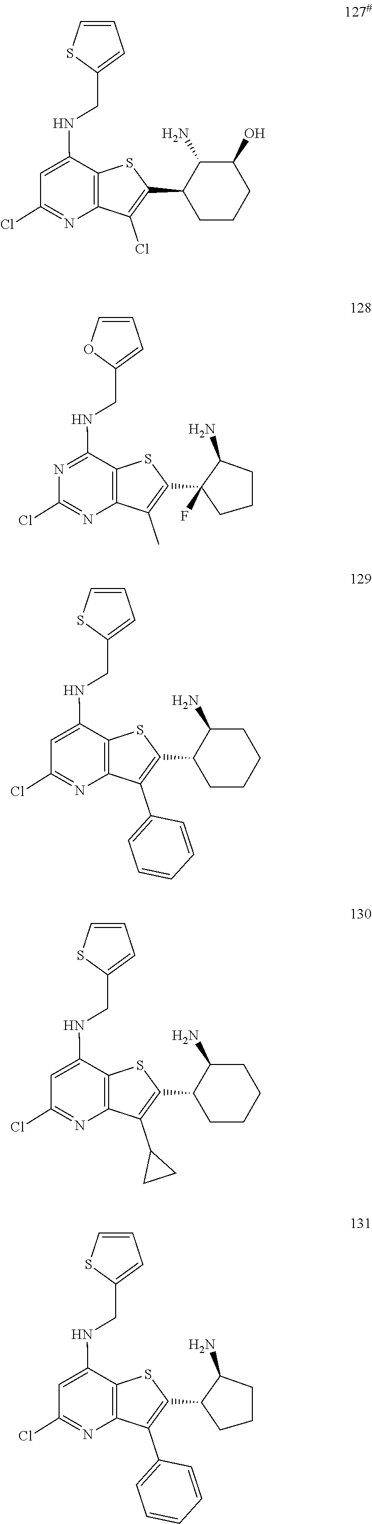 [SMILES: N[C@@H]1[C@@H](O)CCC[C@H]1c1sc2c(NCc3cccs3)cc(Cl)nc2c1Cl ; Cc1c([C@@]2(F)CCC[C@@H]2N)sc2c(NCc3ccco3)nc(Cl)nc12 ; N[C@H]1CCCC[C@@H]1c1sc2c(NCc3cccs3)cc(Cl)nc2c1-c1ccccc1 ; N[C@H]1CCCC[C@@H]1c1sc2c(NCc3cccs3)cc(Cl)nc2c1C1CC1 ; N[C@H]1CCC[C@@H]1c1sc2c(NCc3cccs3)cc(Cl)nc2c1-c1ccccc1]

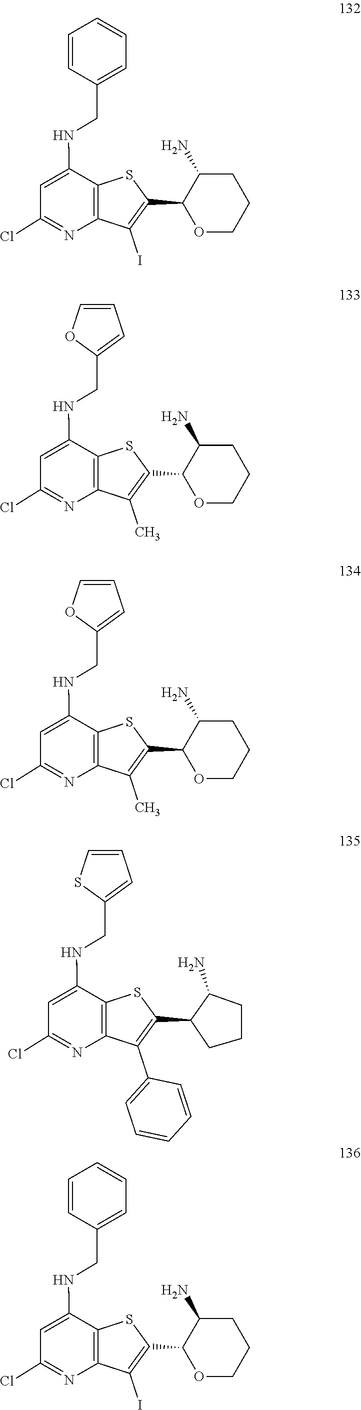

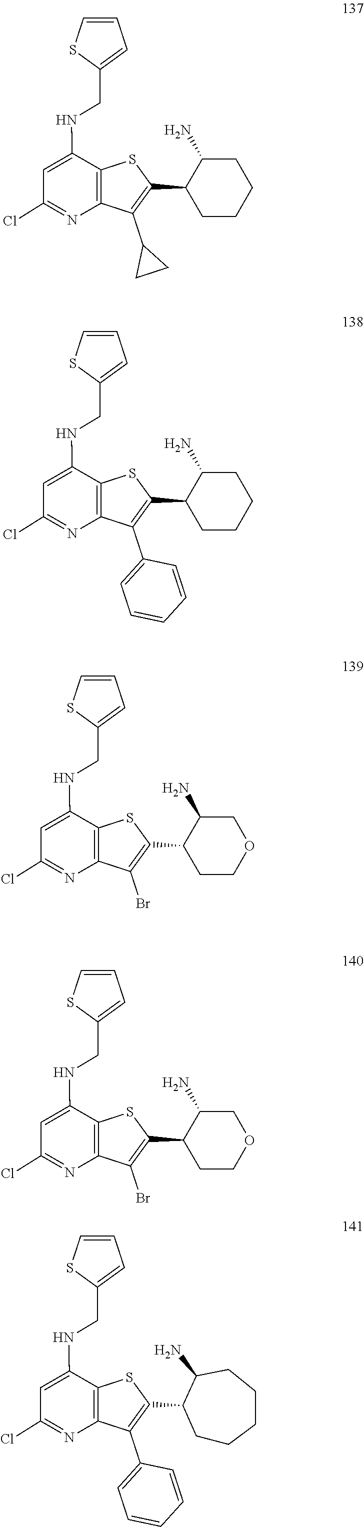

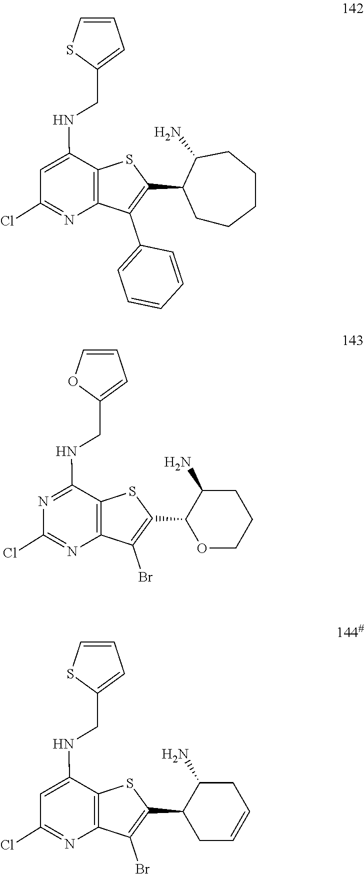 [SMILES: N[C@@H]1CCCCC[C@H]1c1sc2c(NCc3cccs3)cc(Cl)nc2c1-c1ccccc1 ; N[C@H]1CCCO[C@@H]1c1sc2c(NCc3ccco3)nc(Cl)nc2c1Br ; N[C@@H]1CC=CC[C@H]1c1sc2c(NCc3cccs3)cc(Cl)nc2c1Br]

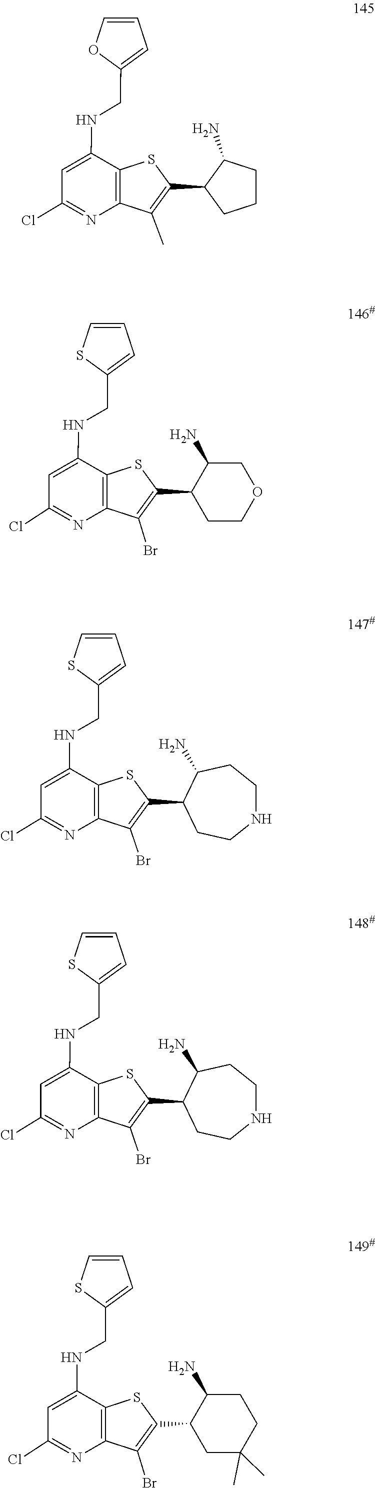 [SMILES: Cc1c([C@@H]2CCC[C@H]2N)sc2c(NCc3ccco3)cc(Cl)nc12 ; N[C@H]1COCC[C@H]1c1sc2c(NCc3cccs3)cc(Cl)nc2c1Br ; N[C@@H]1CCNCC[C@H]1c1sc2c(NCc3cccs3)cc(Cl)nc2c1Br ; N[C@H]1CCNCC[C@H]1c1sc2c(NCc3cccs3)cc(Cl)nc2c1Br ; CC1(C)CC[C@H](N)[C@@H](c2sc3c(NCc4cccs4)cc(Cl)nc3c2Br)C1]

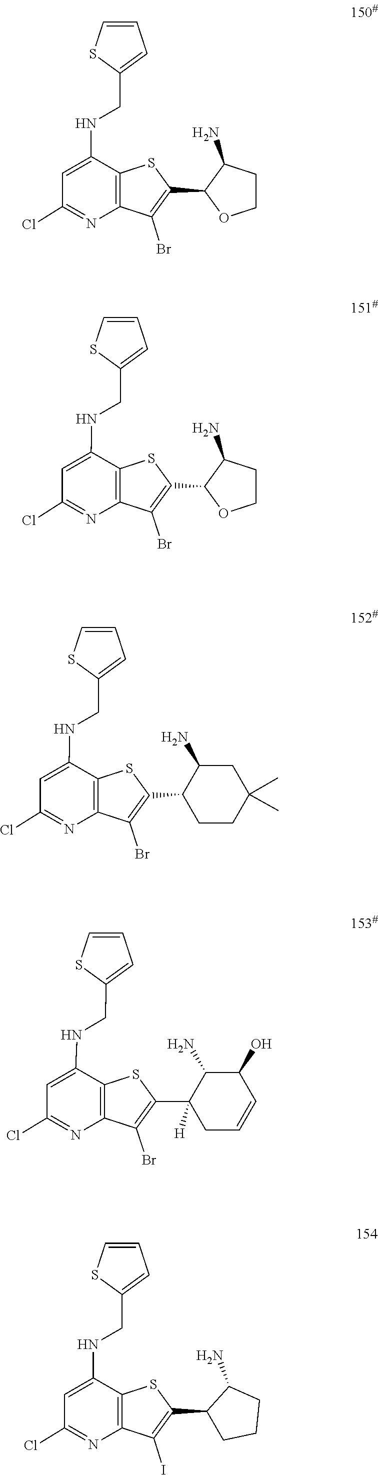 [SMILES: N[C@H]1CCO[C@H]1c1sc2c(NCc3cccs3)cc(Cl)nc2c1Br ; N[C@H]1CCO[C@@H]1c1sc2c(NCc3cccs3)cc(Cl)nc2c1Br ; CC1(C)CC[C@H](c2sc3c(NCc4cccs4)cc(Cl)nc3c2Br)[C@@H](N)C1 ; N[C@@H]1[C@@H](O)C=CC[C@H]1c1sc2c(NCc3cccs3)cc(Cl)nc2c1Br ; N[C@@H]1CCC[C@H]1c1sc2c(NCc3cccs3)cc(Cl)nc2c1I]

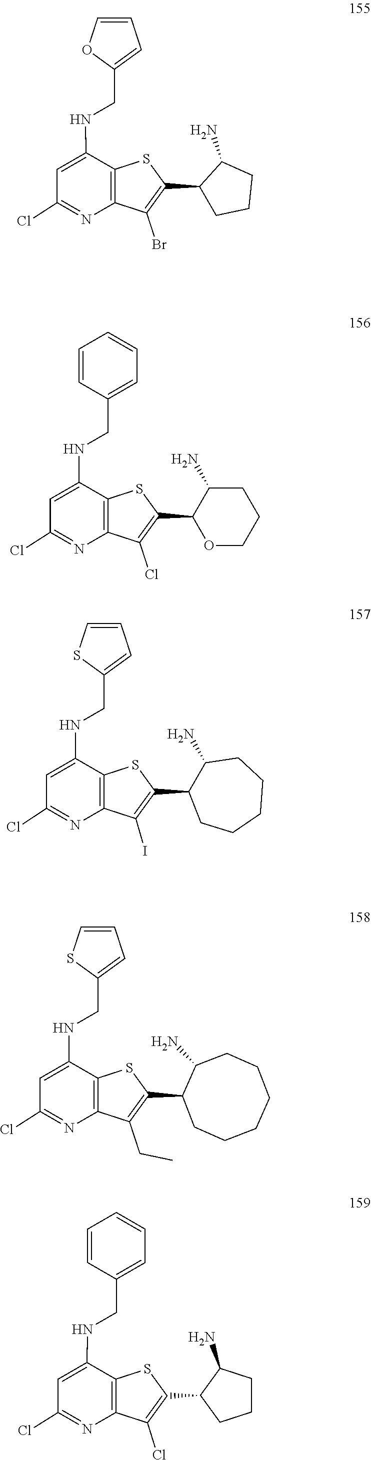 [SMILES: N[C@@H]1CCC[C@H]1c1sc2c(NCc3ccco3)cc(Cl)nc2c1Br ; N[C@@H]1CCCO[C@H]1c1sc2c(NCc3ccccc3)cc(Cl)nc2c1Cl ; N[C@@H]1CCCCC[C@H]1c1sc2c(NCc3cccs3)cc(Cl)nc2c1I ; CCc1c([C@@H]2CCCCCC[C@H]2N)sc2c(NCc3cccs3)cc(Cl)nc12 ; N[C@H]1CCC[C@@H]1c1sc2c(NCc3ccccc3)cc(Cl)nc2c1Cl]

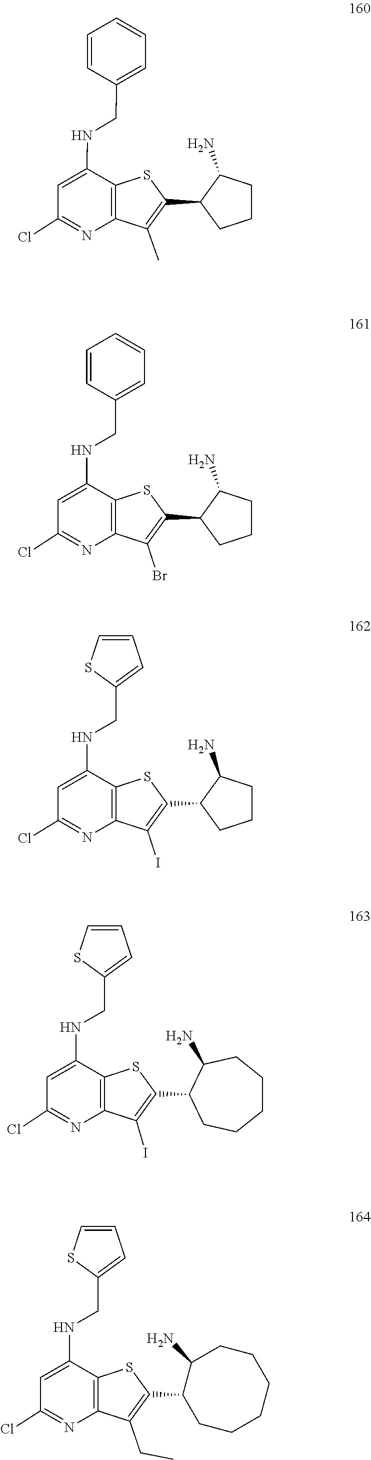 [SMILES: Cc1c([C@@H]2CCC[C@H]2N)sc2c(NCc3ccccc3)cc(Cl)nc12 ; N[C@@H]1CCC[C@H]1c1sc2c(NCc3ccccc3)cc(Cl)nc2c1Br ; N[C@H]1CCC[C@@H]1c1sc2c(NCc3cccs3)cc(Cl)nc2c1I ; N[C@H]1CCCCC[C@@H]1c1sc2c(NCc3cccs3)cc(Cl)nc2c1I ; CCc1c([C@H]2CCCCCC[C@@H]2N)sc2c(NCc3cccs3)cc(Cl)nc12]

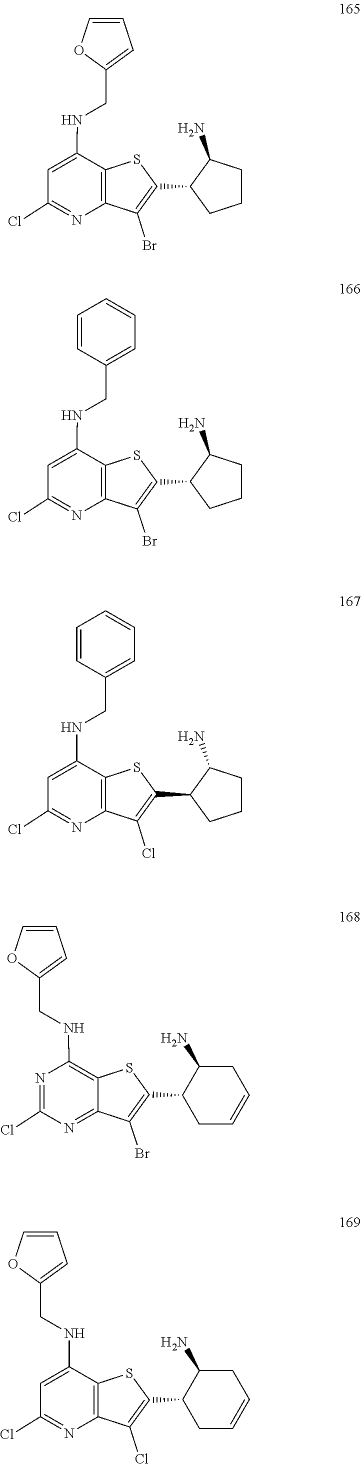 [SMILES: N[C@H]1CCC[C@@H]1c1sc2c(NCc3ccco3)cc(Cl)nc2c1Br ; N[C@H]1CCC[C@@H]1c1sc2c(NCc3ccccc3)cc(Cl)nc2c1Br ; N[C@@H]1CCC[C@H]1c1sc2c(NCc3ccccc3)cc(Cl)nc2c1Cl ; N[C@H]1CC=CC[C@@H]1c1sc2c(NCc3ccco3)nc(Cl)nc2c1Br ; N[C@H]1CC=CC[C@@H]1c1sc2c(NCc3ccco3)cc(Cl)nc2c1Cl]

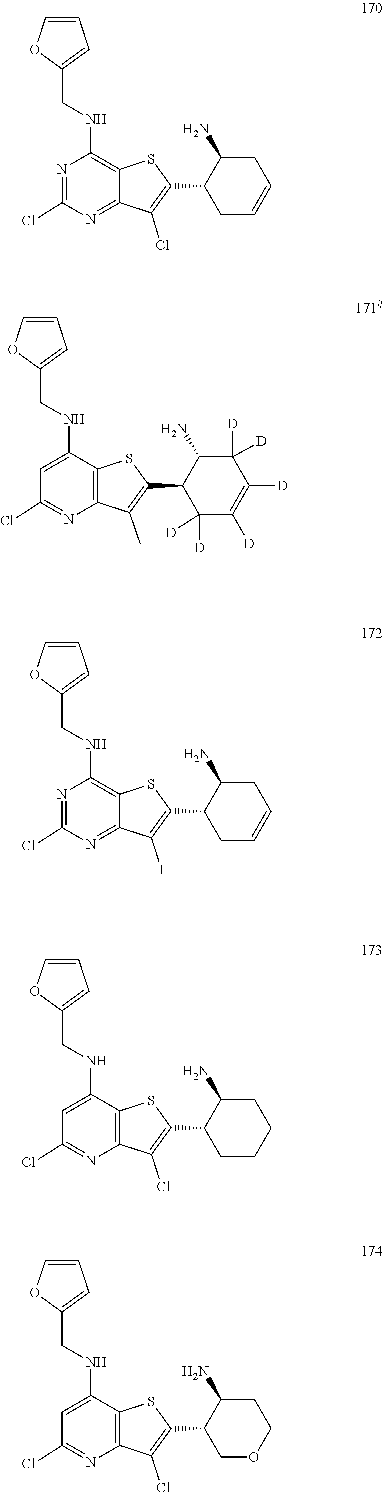 [SMILES: N[C@H]1CC=CC[C@@H]1c1sc2c(NCc3ccco3)nc(Cl)nc2c1Cl ; [2H]C1=C([2H])C([2H])([2H])[C@@H](c2sc3c(NCc4ccco4)cc(Cl)nc3c2C)[C@H](N)C1([2H])[2H] ; N[C@H]1CC=CC[C@@H]1c1sc2c(NCc3ccco3)nc(Cl)nc2c1I ; N[C@H]1CCCC[C@@H]1c1sc2c(NCc3ccco3)cc(Cl)nc2c1Cl ; N[C@H]1CCOC[C@@H]1c1sc2c(NCc3ccco3)cc(Cl)nc2c1Cl]

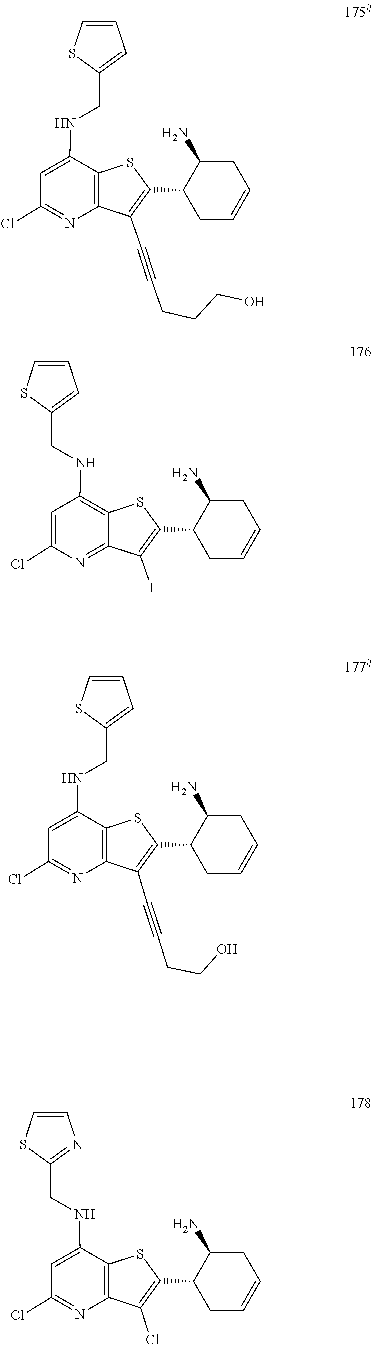 [SMILES: N[C@H]1CC=CC[C@@H]1c1sc2c(NCc3cccs3)cc(Cl)nc2c1C#CCCCO ; N[C@H]1CC=CC[C@@H]1c1sc2c(NCc3cccs3)cc(Cl)nc2c1I ; N[C@H]1CC=CC[C@@H]1c1sc2c(NCc3cccs3)cc(Cl)nc2c1C#CCCO ; N[C@H]1CC=CC[C@@H]1c1sc2c(NCc3nccs3)cc(Cl)nc2c1Cl]

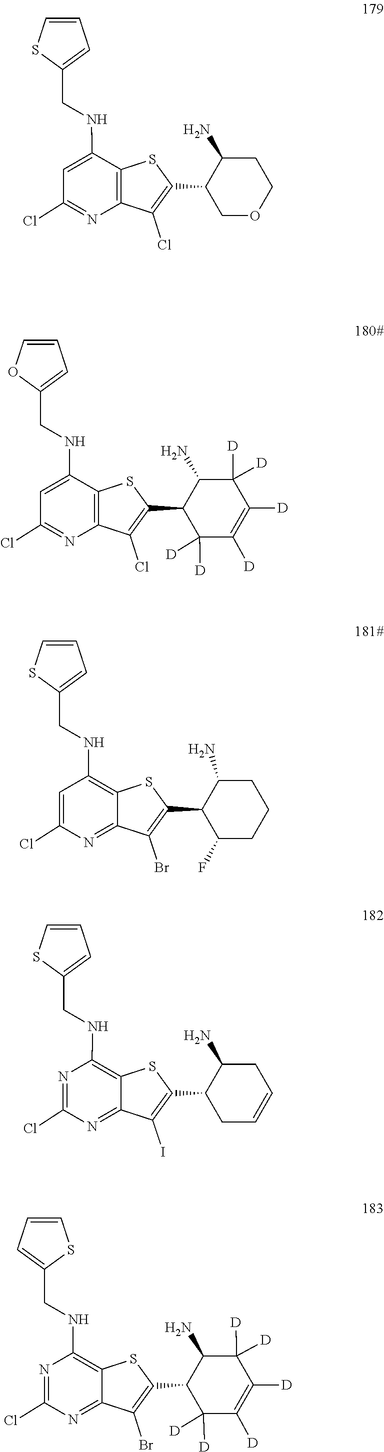 [SMILES: N[C@H]1CCOC[C@@H]1c1sc2c(NCc3cccs3)cc(Cl)nc2c1Cl ; [2H]C1=C([2H])C([2H])([2H])[C@@H](c2sc3c(NCc4ccco4)cc(Cl)nc3c2Cl)[C@H](N)C1([2H])[2H] ; N[C@@H]1CCC[C@H](F)[C@H]1c1sc2c(NCc3cccs3)cc(Cl)nc2c1Br ; N[C@H]1CC=CC[C@@H]1c1sc2c(NCc3cccs3)nc(Cl)nc2c1I ; [2H]C1=C([2H])C([2H])([2H])[C@H](c2sc3c(NCc4cccs4)nc(Cl)nc3c2Br)[C@@H](N)C1([2H])[2H]]

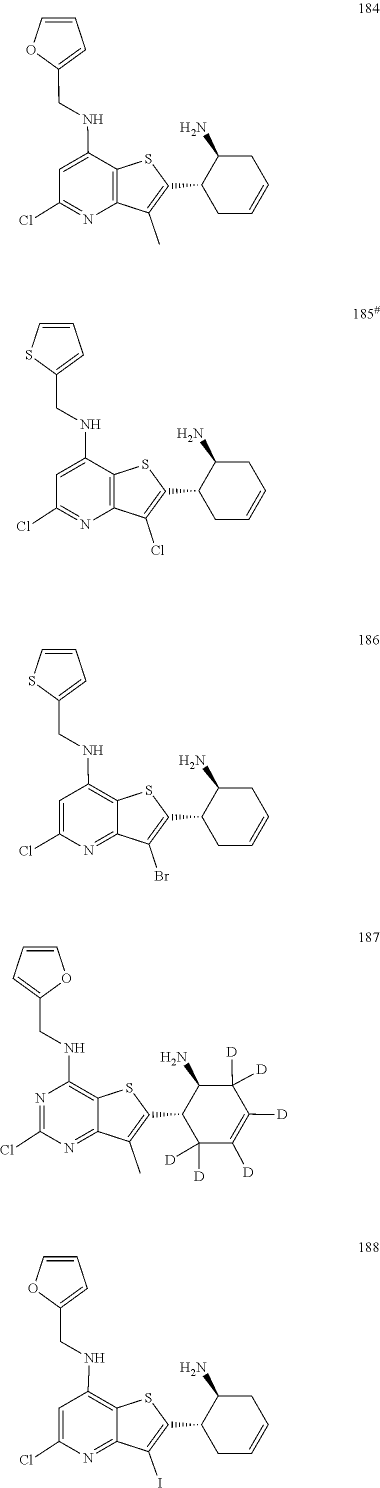 [SMILES: Cc1c([C@H]2CC=CC[C@@H]2N)sc2c(NCc3ccco3)cc(Cl)nc12 ; N[C@H]1CC=CC[C@@H]1c1sc2c(NCc3cccs3)cc(Cl)nc2c1Cl ; N[C@H]1CC=CC[C@@H]1c1sc2c(NCc3cccs3)cc(Cl)nc2c1Br ; [2H]C1=C([2H])C([2H])([2H])[C@H](c2sc3c(NCc4ccco4)nc(Cl)nc3c2C)[C@@H](N)C1([2H])[2H] ; N[C@H]1CC=CC[C@@H]1c1sc2c(NCc3ccco3)cc(Cl)nc2c1I]

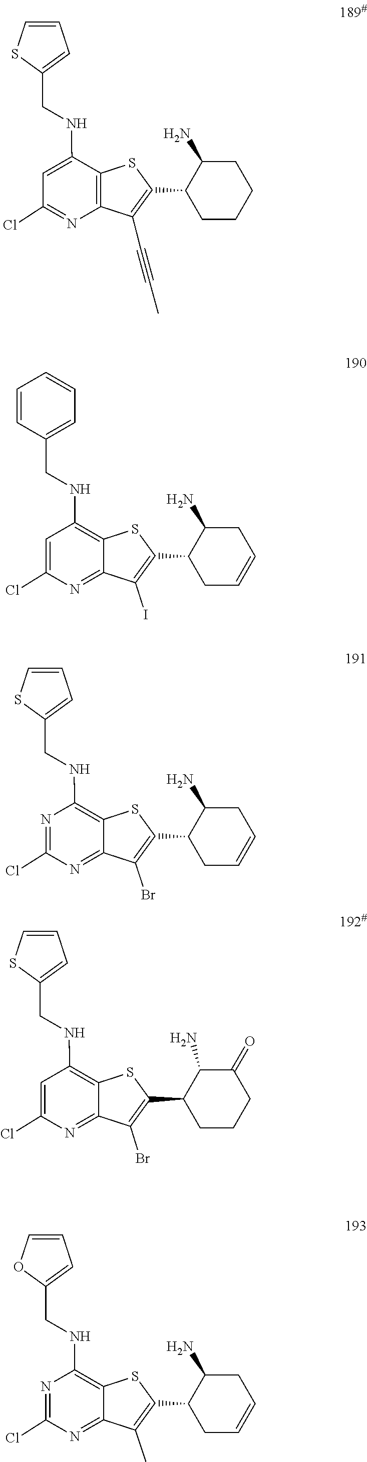 [SMILES: CC#Cc1c([C@H]2CCCC[C@@H]2N)sc2c(NCc3cccs3)cc(Cl)nc12 ; N[C@H]1CC=CC[C@@H]1c1sc2c(NCc3ccccc3)cc(Cl)nc2c1I ; N[C@H]1CC=CC[C@@H]1c1sc2c(NCc3cccs3)nc(Cl)nc2c1Br ; N[C@@H]1C(=O)CCC[C@H]1c1sc2c(NCc3cccs3)cc(Cl)nc2c1Br ; Cc1c([C@H]2CC=CC[C@@H]2N)sc2c(NCc3ccco3)nc(Cl)nc12]

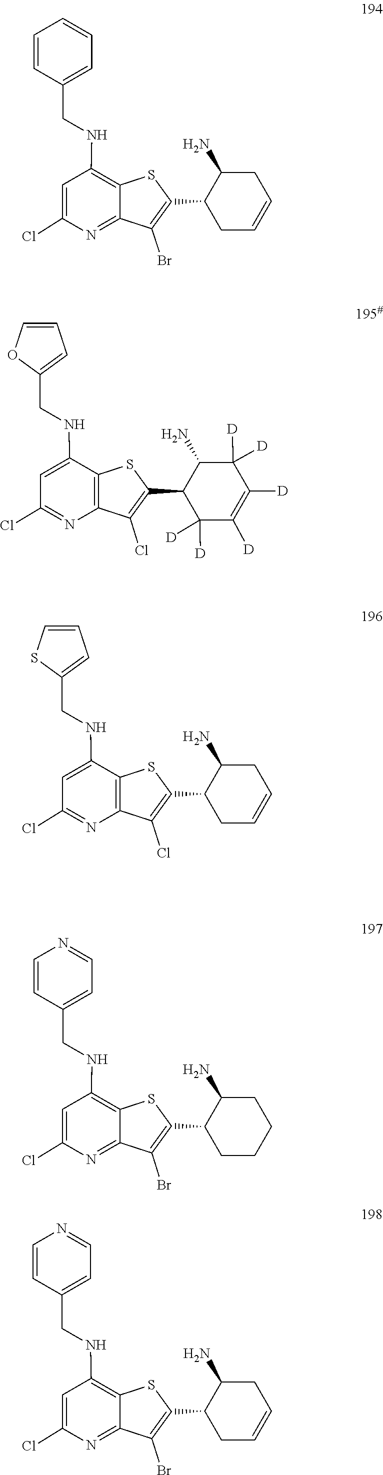 [SMILES: N[C@H]1CC=CC[C@@H]1c1sc2c(NCc3ccccc3)cc(Cl)nc2c1Br ; [2H]C1=C([2H])C([2H])([2H])[C@@H](c2sc3c(NCc4ccco4)cc(Cl)nc3c2Cl)[C@H](N)C1([2H])[2H] ; N[C@H]1CC=CC[C@@H]1c1sc2c(NCc3cccs3)cc(Cl)nc2c1Cl ; N[C@H]1CCCC[C@@H]1c1sc2c(NCc3ccncc3)cc(Cl)nc2c1Br ; N[C@H]1CC=CC[C@@H]1c1sc2c(NCc3ccncc3)cc(Cl)nc2c1Br]

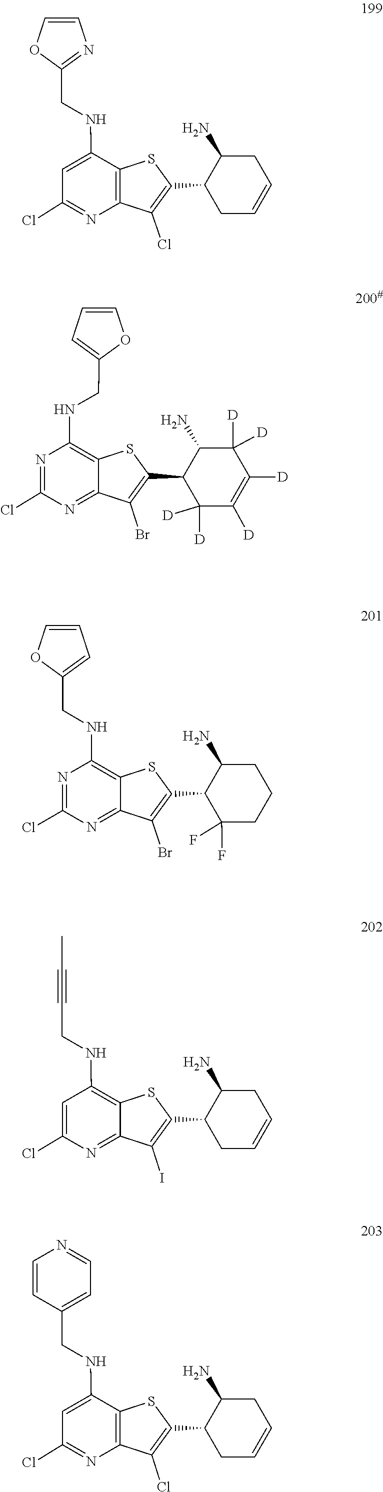 [SMILES: N[C@H]1CC=CC[C@@H]1c1sc2c(NCc3ncco3)cc(Cl)nc2c1Cl ; [2H]C1=C([2H])C([2H])([2H])[C@@H](c2sc3c(NCc4ccco4)nc(Cl)nc3c2Br)[C@H](N)C1([2H])[2H] ; N[C@H]1CCCC(F)(F)[C@@H]1c1sc2c(NCc3ccco3)nc(Cl)nc2c1Br ; CC#CCNc1cc(Cl)nc2c(I)c([C@H]3CC=CC[C@@H]3N)sc12 ; N[C@H]1CC=CC[C@@H]1c1sc2c(NCc3ccncc3)cc(Cl)nc2c1Cl]

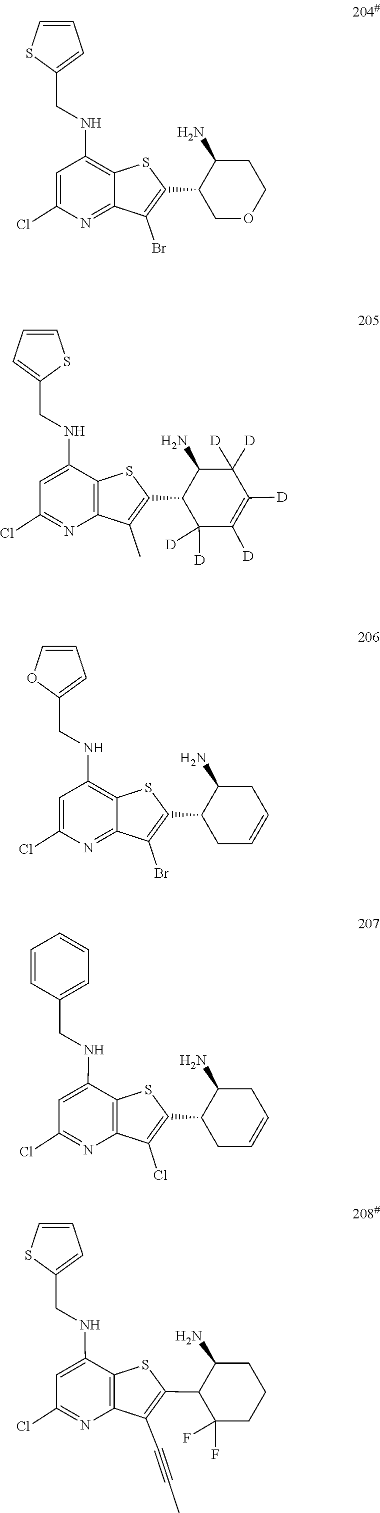 [SMILES: N[C@H]1CCOC[C@@H]1c1sc2c(NCc3cccs3)cc(Cl)nc2c1Br ; [2H]C1=C([2H])C([2H])([2H])[C@H](c2sc3c(NCc4cccs4)cc(Cl)nc3c2C)[C@@H](N)C1([2H])[2H] ; N[C@H]1CC=CC[C@@H]1c1sc2c(NCc3ccco3)cc(Cl)nc2c1Br ; N[C@H]1CC=CC[C@@H]1c1sc2c(NCc3ccccc3)cc(Cl)nc2c1Cl ; CC#Cc1c(C2[C@@H](N)CCCC2(F)F)sc2c(NCc3cccs3)cc(Cl)nc12]

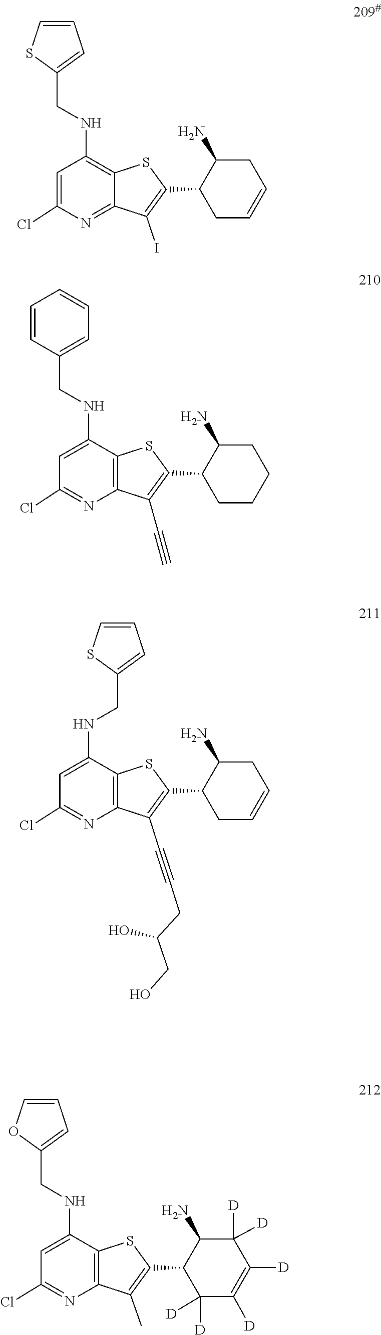 [SMILES: N[C@H]1CC=CC[C@@H]1c1sc2c(NCc3cccs3)cc(Cl)nc2c1I ; C#Cc1c([C@H]2CCCC[C@@H]2N)sc2c(NCc3ccccc3)cc(Cl)nc12 ; N[C@H]1CC=CC[C@@H]1c1sc2c(NCc3cccs3)cc(Cl)nc2c1C#CC[C@@H](O)CO ; [2H]C1=C([2H])C([2H])([2H])[C@H](c2sc3c(NCc4ccco4)cc(Cl)nc3c2C)[C@@H](N)C1([2H])[2H]]

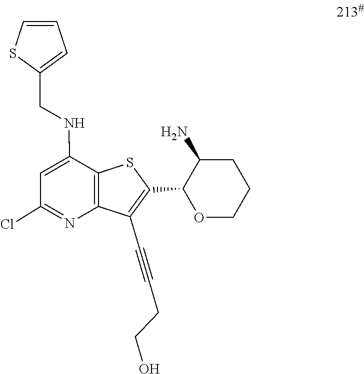 [SMILES: N[C@H]1CCCO[C@@H]1c1sc2c(NCc3cccs3)cc(Cl)nc2c1C#CCCO]

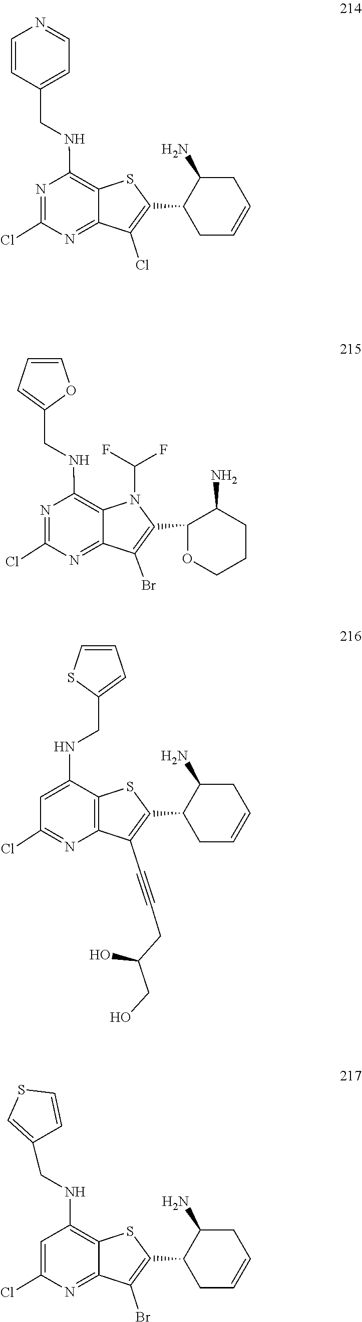 [SMILES: N[C@H]1CC=CC[C@@H]1c1sc2c(NCc3ccncc3)nc(Cl)nc2c1Cl ; N[C@H]1CCCO[C@@H]1c1c(Br)c2nc(Cl)nc(NCc3ccco3)c2n1C(F)F ; N[C@H]1CC=CC[C@@H]1c1sc2c(NCc3cccs3)cc(Cl)nc2c1C#CC[C@H](O)CO ; N[C@H]1CC=CC[C@@H]1c1sc2c(NCc3ccsc3)cc(Cl)nc2c1Br]

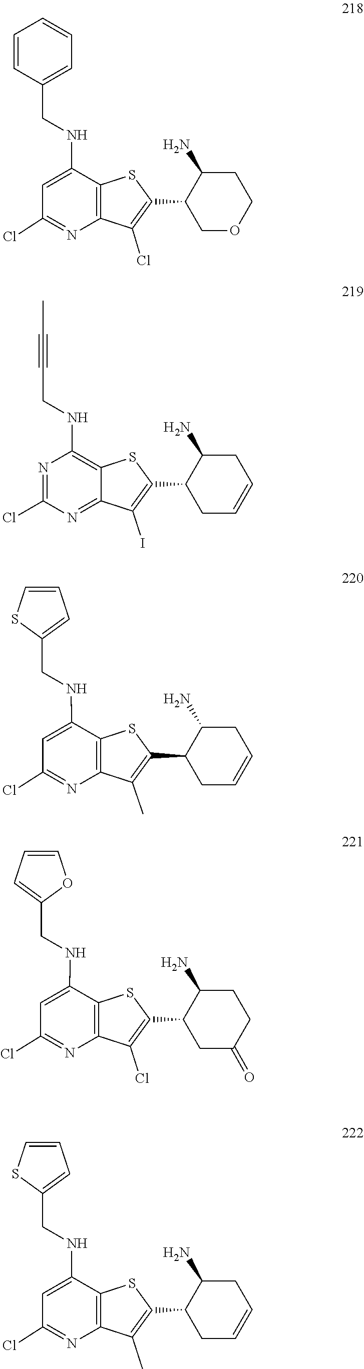 [SMILES: N[C@H]1CCOC[C@@H]1c1sc2c(NCc3ccccc3)cc(Cl)nc2c1Cl ; CC#CCNc1nc(Cl)nc2c(I)c([C@H]3CC=CC[C@@H]3N)sc12 ; Cc1c([C@@H]2CC=CC[C@H]2N)sc2c(NCc3cccs3)cc(Cl)nc12 ; N[C@H]1CCC(=O)C[C@@H]1c1sc2c(NCc3ccco3)cc(Cl)nc2c1Cl ; Cc1c([C@H]2CC=CC[C@@H]2N)sc2c(NCc3cccs3)cc(Cl)nc12]

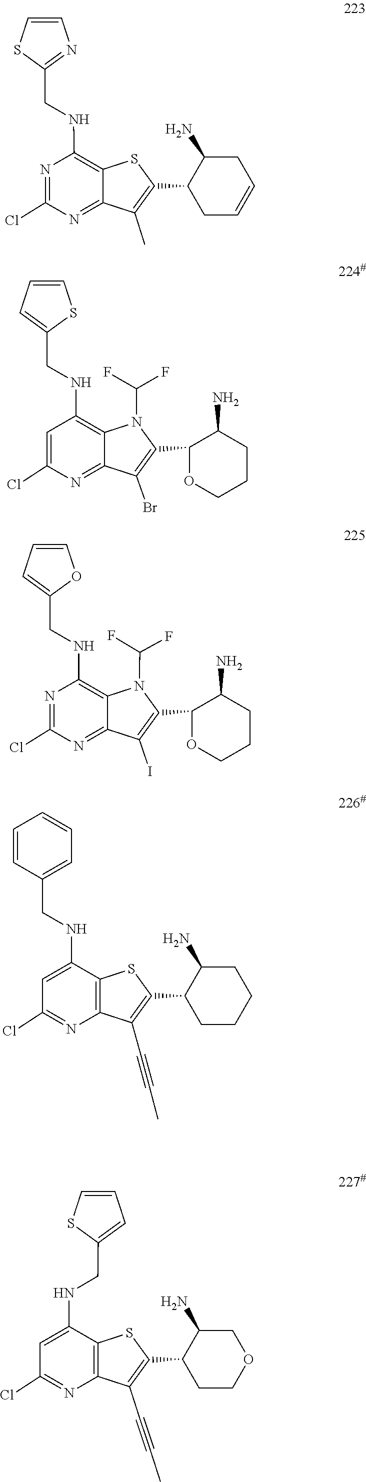 [SMILES: Cc1c([C@H]2CC=CC[C@@H]2N)sc2c(NCc3nccs3)nc(Cl)nc12 ; N[C@H]1CCCO[C@@H]1c1c(Br)c2nc(Cl)cc(NCc3cccs3)c2n1C(F)F ; N[C@H]1CCCO[C@@H]1c1c(I)c2nc(Cl)nc(NCc3ccco3)c2n1C(F)F ; CC#Cc1c([C@H]2CCCC[C@@H]2N)sc2c(NCc3ccccc3)cc(Cl)nc12 ; CC#Cc1c([C@H]2CCOC[C@@H]2N)sc2c(NCc3cccs3)cc(Cl)nc12]

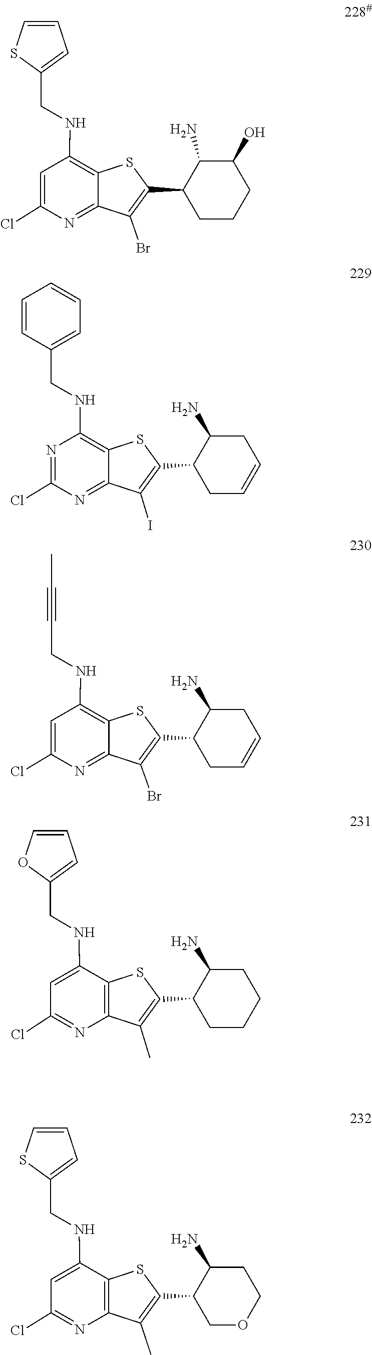 [SMILES: N[C@@H]1[C@@H](O)CCC[C@H]1c1sc2c(NCc3cccs3)cc(Cl)nc2c1Br ; N[C@H]1CC=CC[C@@H]1c1sc2c(NCc3ccccc3)nc(Cl)nc2c1I ; CC#CCNc1cc(Cl)nc2c(Br)c([C@H]3CC=CC[C@@H]3N)sc12 ; Cc1c([C@H]2CCCC[C@@H]2N)sc2c(NCc3ccco3)cc(Cl)nc12 ; Cc1c([C@H]2COCC[C@@H]2N)sc2c(NCc3cccs3)cc(Cl)nc12]

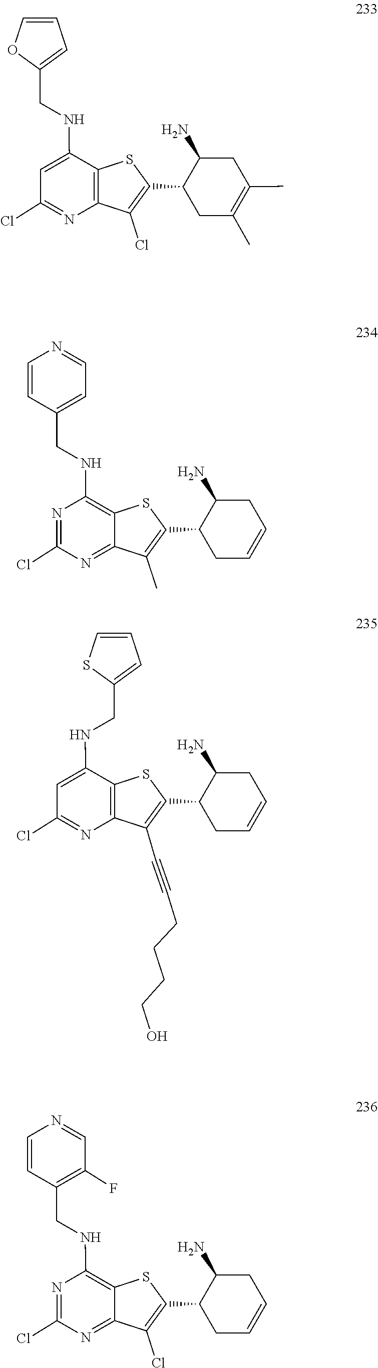 [SMILES: CC1=C(C)C[C@H](N)[C@@H](c2sc3c(NCc4ccco4)cc(Cl)nc3c2Cl)C1 ; Cc1c([C@H]2CC=CC[C@@H]2N)sc2c(NCc3ccncc3)nc(Cl)nc12 ; N[C@H]1CC=CC[C@@H]1c1sc2c(NCc3cccs3)cc(Cl)nc2c1C#CCCCCO ; N[C@H]1CC=CC[C@@H]1c1sc2c(NCc3ccncc3F)nc(Cl)nc2c1Cl]

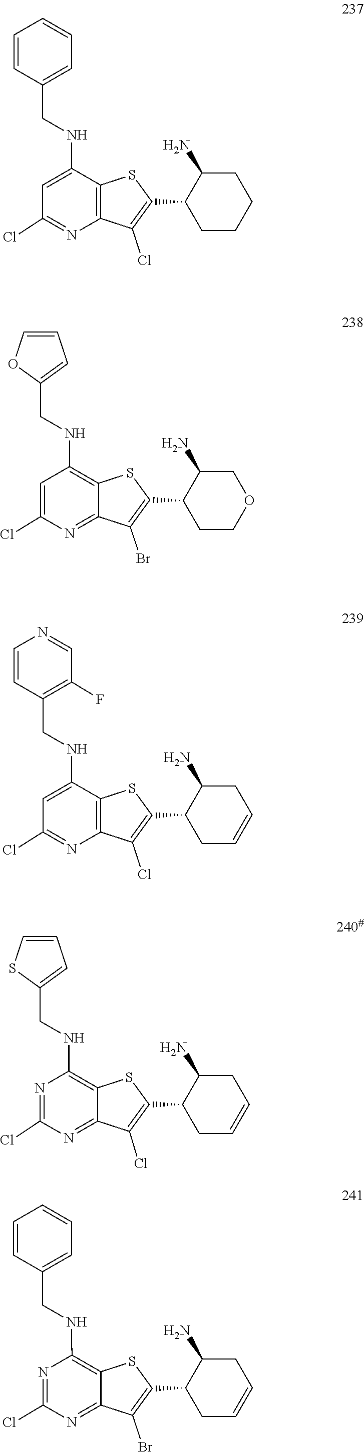 [SMILES: N[C@H]1CCCC[C@@H]1c1sc2c(NCc3ccccc3)cc(Cl)nc2c1Cl ; N[C@H]1COCC[C@@H]1c1sc2c(NCc3ccco3)cc(Cl)nc2c1Br ; N[C@H]1CC=CC[C@@H]1c1sc2c(NCc3ccncc3F)cc(Cl)nc2c1Cl ; N[C@H]1CC=CC[C@@H]1c1sc2c(NCc3cccs3)nc(Cl)nc2c1Cl ; N[C@H]1CC=CC[C@@H]1c1sc2c(NCc3ccccc3)nc(Cl)nc2c1Br]

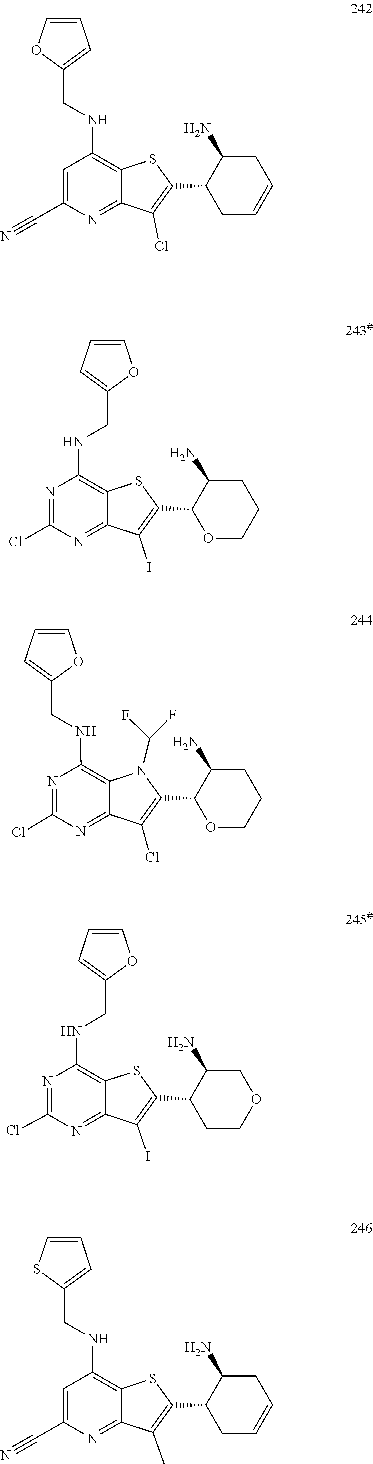 [SMILES: N#Cc1cc(NCc2ccco2)c2sc([C@H]3CC=CC[C@@H]3N)c(Cl)c2n1 ; N[C@H]1CCCO[C@@H]1c1sc2c(NCc3ccco3)nc(Cl)nc2c1I ; N[C@H]1CCCO[C@@H]1c1c(Cl)c2nc(Cl)nc(NCc3ccco3)c2n1C(F)F ; N[C@H]1COCC[C@@H]1c1sc2c(NCc3ccco3)nc(Cl)nc2c1I ; Cc1c([C@H]2CC=CC[C@@H]2N)sc2c(NCc3cccs3)cc(C#N)nc12]

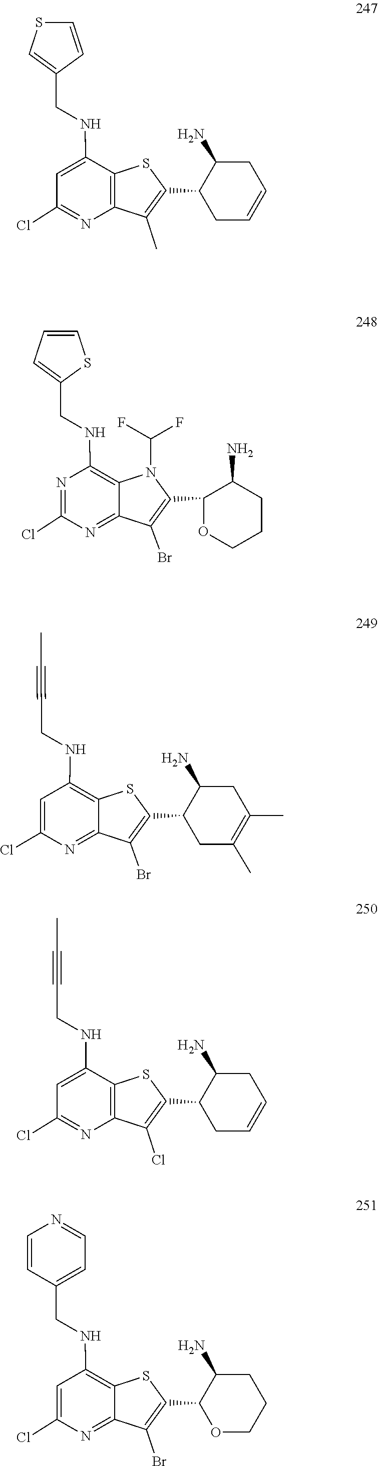 [SMILES: Cc1c([C@H]2CC=CC[C@@H]2N)sc2c(NCc3ccsc3)cc(Cl)nc12 ; N[C@H]1CCCO[C@@H]1c1c(Br)c2nc(Cl)nc(NCc3cccs3)c2n1C(F)F ; CC#CCNc1cc(Cl)nc2c(Br)c([C@H]3CC(C)=C(C)C[C@@H]3N)sc12 ; CC#CCNc1cc(Cl)nc2c(Cl)c([C@H]3CC=CC[C@@H]3N)sc12 ; N[C@H]1CCCO[C@@H]1c1sc2c(NCc3ccncc3)cc(Cl)nc2c1Br]

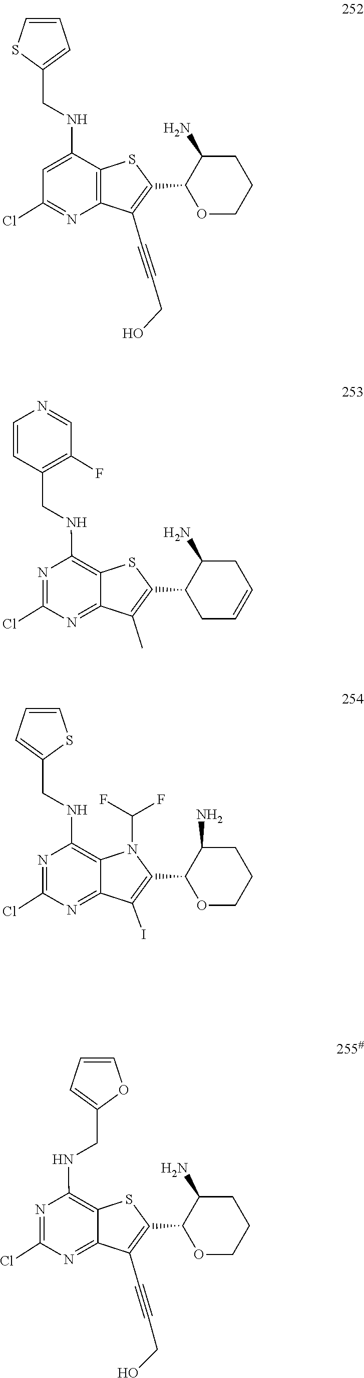 [SMILES: N[C@H]1CCCO[C@@H]1c1sc2c(NCc3cccs3)cc(Cl)nc2c1C#CCO ; Cc1c([C@H]2CC=CC[C@@H]2N)sc2c(NCc3ccncc3F)nc(Cl)nc12 ; N[C@H]1CCCO[C@@H]1c1c(I)c2nc(Cl)nc(NCc3cccs3)c2n1C(F)F ; N[C@H]1CCCO[C@@H]1c1sc2c(NCc3ccco3)nc(Cl)nc2c1C#CCO]

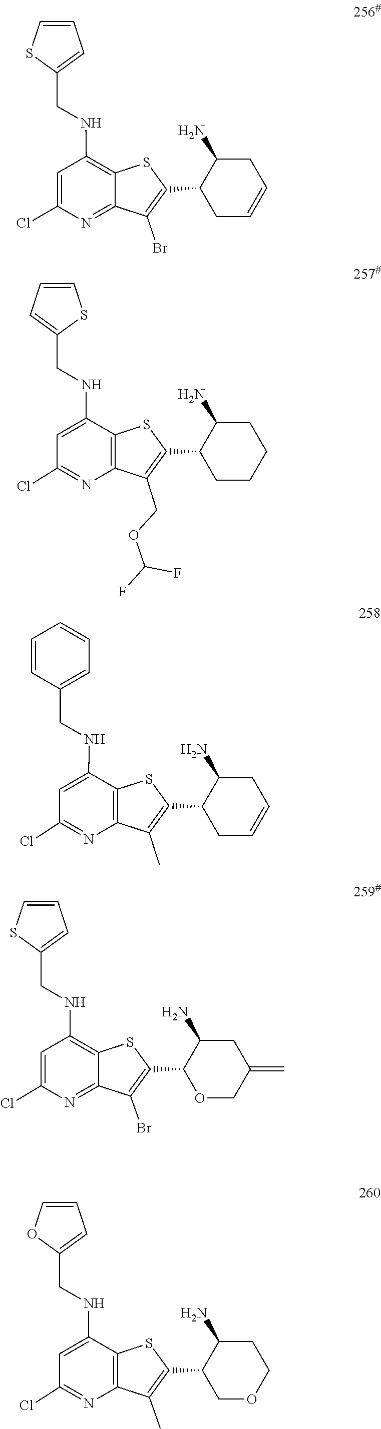 [SMILES: N[C@H]1CC=CC[C@@H]1c1sc2c(NCc3cccs3)cc(Cl)nc2c1Br ; N[C@H]1CCCC[C@@H]1c1sc2c(NCc3cccs3)cc(Cl)nc2c1COC(F)F ; Cc1c([C@H]2CC=CC[C@@H]2N)sc2c(NCc3ccccc3)cc(Cl)nc12 ; C=C1CO[C@H](c2sc3c(NCc4cccs4)cc(Cl)nc3c2Br)[C@@H](N)C1 ; Cc1c([C@H]2COCC[C@@H]2N)sc2c(NCc3ccco3)cc(Cl)nc12]

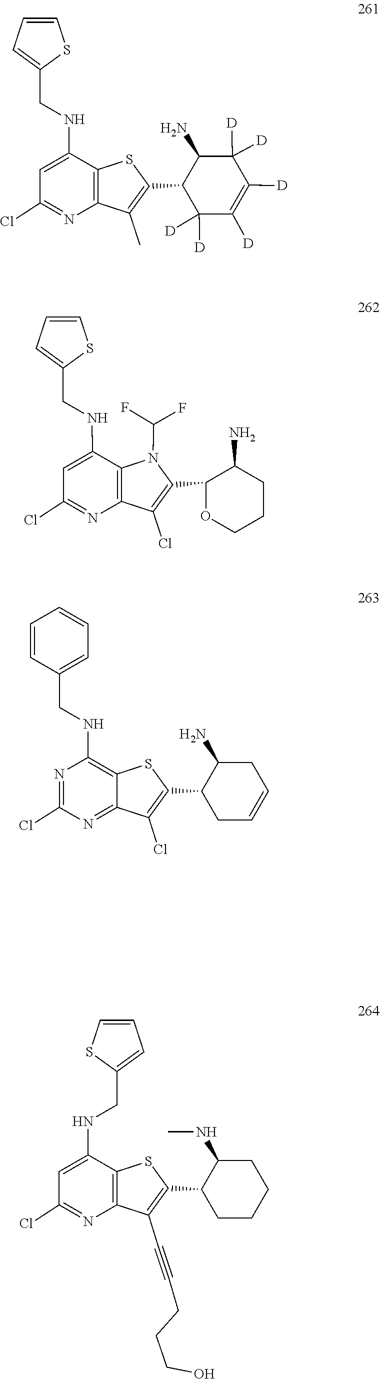 [SMILES: [2H]C1=C([2H])C([2H])([2H])[C@H](c2sc3c(NCc4cccs4)cc(Cl)nc3c2C)[C@@H](N)C1([2H])[2H] ; N[C@H]1CCCO[C@@H]1c1c(Cl)c2nc(Cl)cc(NCc3cccs3)c2n1C(F)F ; N[C@H]1CC=CC[C@@H]1c1sc2c(NCc3ccccc3)nc(Cl)nc2c1Cl ; CN[C@H]1CCCC[C@@H]1c1sc2c(NCc3cccs3)cc(Cl)nc2c1C#CCCCO]

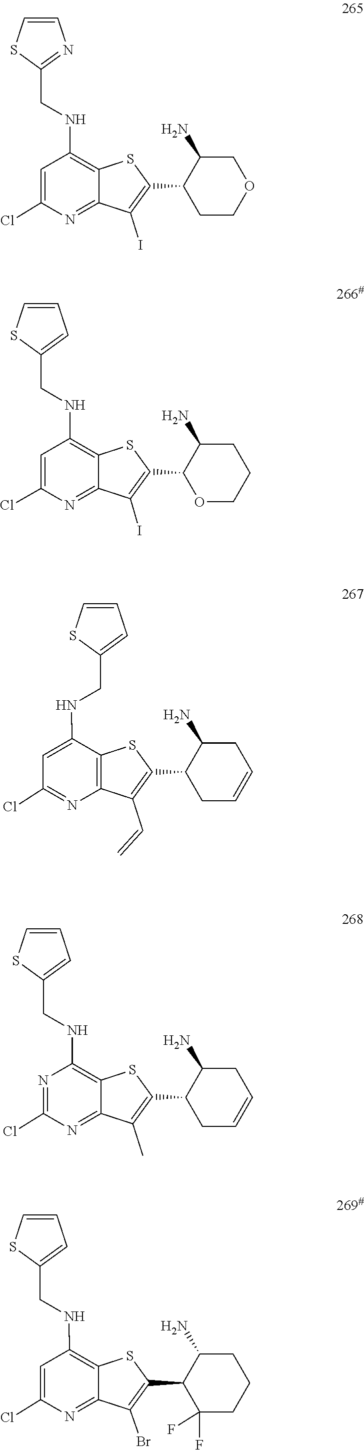 [SMILES: N[C@H]1COCC[C@@H]1c1sc2c(NCc3nccs3)cc(Cl)nc2c1I ; N[C@H]1CCCO[C@@H]1c1sc2c(NCc3cccs3)cc(Cl)nc2c1I ; C=Cc1c([C@H]2CC=CC[C@@H]2N)sc2c(NCc3cccs3)cc(Cl)nc12 ; Cc1c([C@H]2CC=CC[C@@H]2N)sc2c(NCc3cccs3)nc(Cl)nc12 ; N[C@@H]1CCCC(F)(F)[C@H]1c1sc2c(NCc3cccs3)cc(Cl)nc2c1Br]

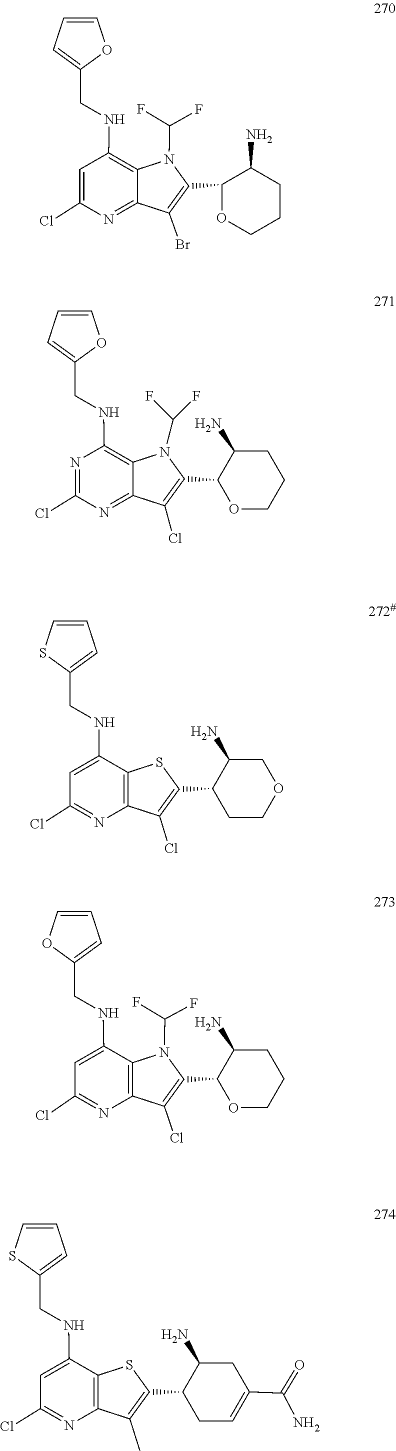 [SMILES: N[C@H]1CCCO[C@@H]1c1c(Br)c2nc(Cl)cc(NCc3ccco3)c2n1C(F)F ; N[C@H]1CCCO[C@@H]1c1c(Cl)c2nc(Cl)nc(NCc3ccco3)c2n1C(F)F ; N[C@H]1COCC[C@@H]1c1sc2c(NCc3cccs3)cc(Cl)nc2c1Cl ; N[C@H]1CCCO[C@@H]1c1c(Cl)c2nc(Cl)cc(NCc3ccco3)c2n1C(F)F ; Cc1c([C@H]2CC=C(C(N)=O)C[C@@H]2N)sc2c(NCc3cccs3)cc(Cl)nc12]

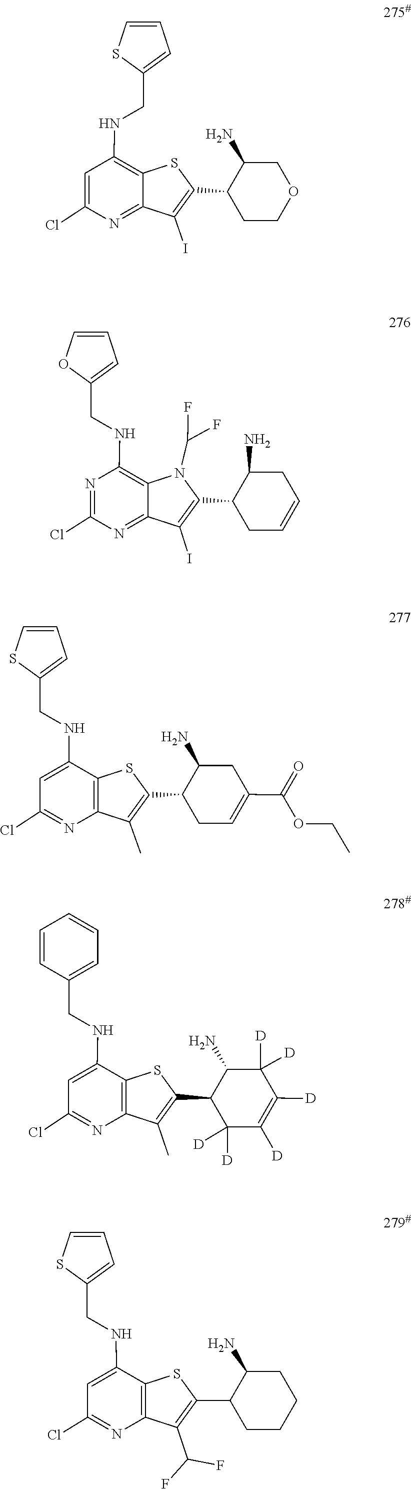 [SMILES: N[C@H]1COCC[C@@H]1c1sc2c(NCc3cccs3)cc(Cl)nc2c1I ; N[C@H]1CC=CC[C@@H]1c1c(I)c2nc(Cl)nc(NCc3ccco3)c2n1C(F)F ; CCOC(=O)C1=CC[C@H](c2sc3c(NCc4cccs4)cc(Cl)nc3c2C)[C@@H](N)C1 ; [2H]C1=C([2H])C([2H])([2H])[C@@H](c2sc3c(NCc4ccccc4)cc(Cl)nc3c2C)[C@H](N)C1([2H])[2H] ; N[C@H]1CCCCC1c1sc2c(NCc3cccs3)cc(Cl)nc2c1C(F)F]

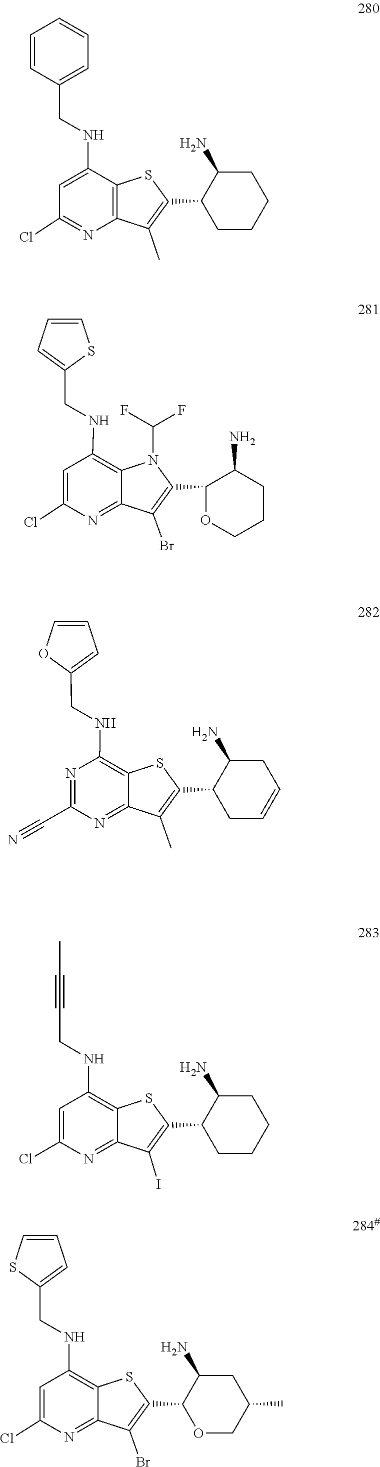 [SMILES: Cc1c([C@H]2CCCC[C@@H]2N)sc2c(NCc3ccccc3)cc(Cl)nc12 ; N[C@H]1CCCO[C@@H]1c1c(Br)c2nc(Cl)cc(NCc3cccs3)c2n1C(F)F ; Cc1c([C@H]2CC=CC[C@@H]2N)sc2c(NCc3ccco3)nc(C#N)nc12 ; CC#CCNc1cc(Cl)nc2c(I)c([C@H]3CCCC[C@@H]3N)sc12 ; C[C@@H]1CO[C@H](c2sc3c(NCc4cccs4)cc(Cl)nc3c2Br)[C@@H](N)C1]

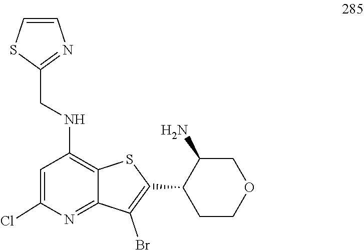 [SMILES: N[C@H]1COCC[C@@H]1c1sc2c(NCc3nccs3)cc(Cl)nc2c1Br]

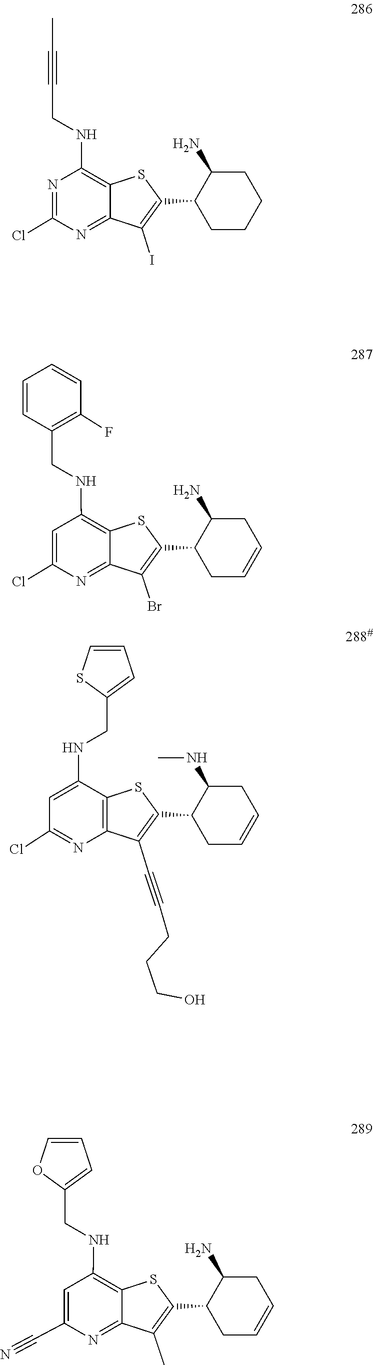 [SMILES: CC#CCNc1nc(Cl)nc2c(I)c([C@H]3CCCC[C@@H]3N)sc12 ; N[C@H]1CC=CC[C@@H]1c1sc2c(NCc3ccccc3F)cc(Cl)nc2c1Br ; CN[C@H]1CC=CC[C@@H]1c1sc2c(NCc3cccs3)cc(Cl)nc2c1C#CCCCO ; Cc1c([C@H]2CC=CC[C@@H]2N)sc2c(NCc3ccco3)cc(C#N)nc12]

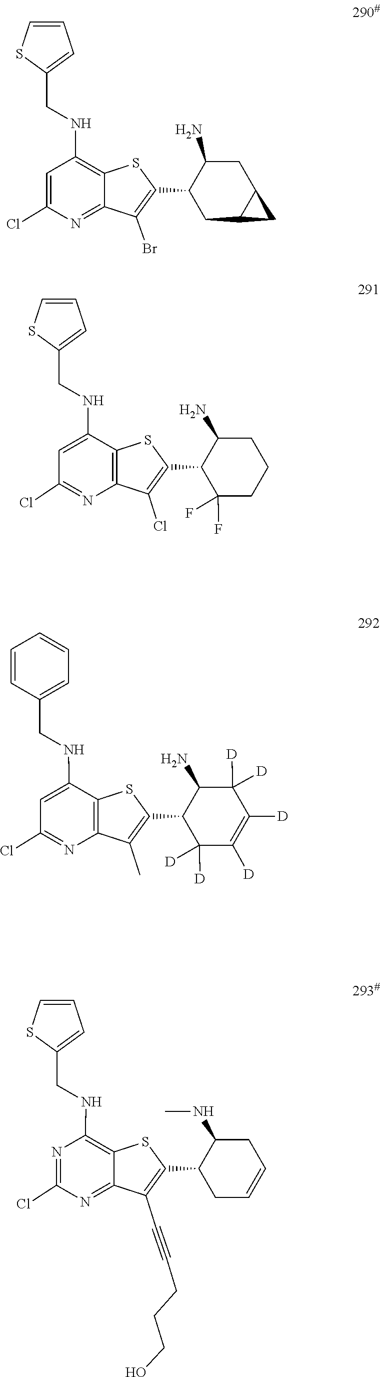

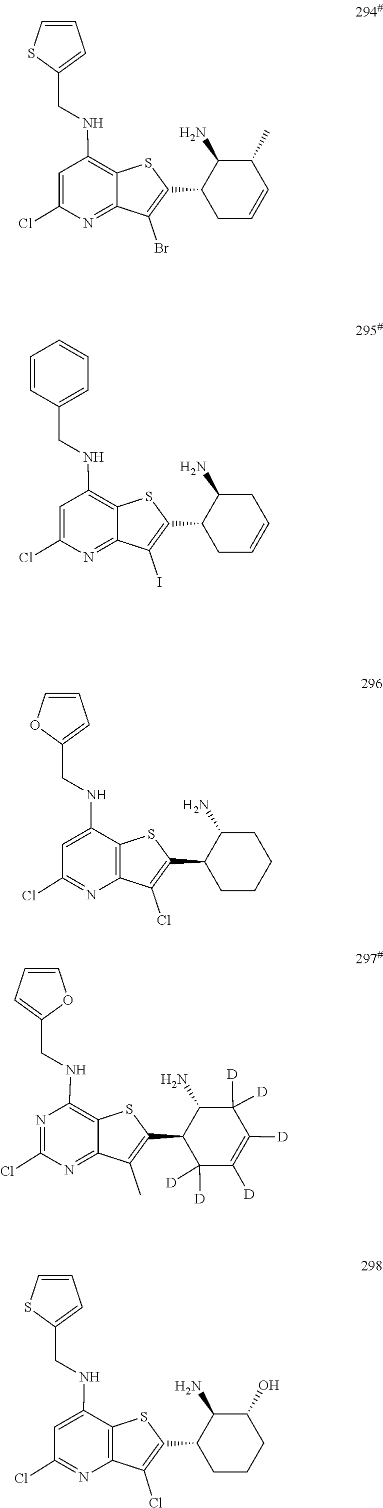 [SMILES: C[C@@H]1C=CC[C@H](c2sc3c(NCc4cccs4)cc(Cl)nc3c2Br)[C@H]1N ; N[C@H]1CC=CC[C@@H]1c1sc2c(NCc3ccccc3)cc(Cl)nc2c1I ; N[C@@H]1CCCC[C@H]1c1sc2c(NCc3ccco3)cc(Cl)nc2c1Cl ; [2H]C1=C([2H])C([2H])([2H])[C@@H](c2sc3c(NCc4ccco4)nc(Cl)nc3c2C)[C@H](N)C1([2H])[2H] ; N[C@H]1[C@H](O)CCC[C@@H]1c1sc2c(NCc3cccs3)cc(Cl)nc2c1Cl]

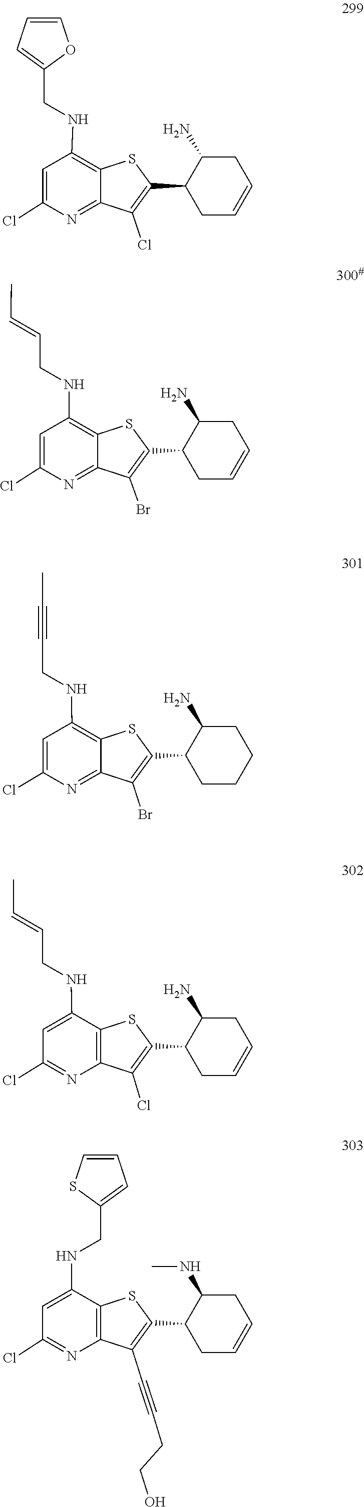 [SMILES: N[C@@H]1CC=CC[C@H]1c1sc2c(NCc3ccco3)cc(Cl)nc2c1Cl ; C/C=C/CNc1cc(Cl)nc2c(Br)c([C@H]3CC=CC[C@@H]3N)sc12 ; CC#CCNc1cc(Cl)nc2c(Br)c([C@H]3CCCC[C@@H]3N)sc12 ; C/C=C/CNc1cc(Cl)nc2c(Cl)c([C@H]3CC=CC[C@@H]3N)sc12 ; CN[C@H]1CC=CC[C@@H]1c1sc2c(NCc3cccs3)cc(Cl)nc2c1C#CCCO]

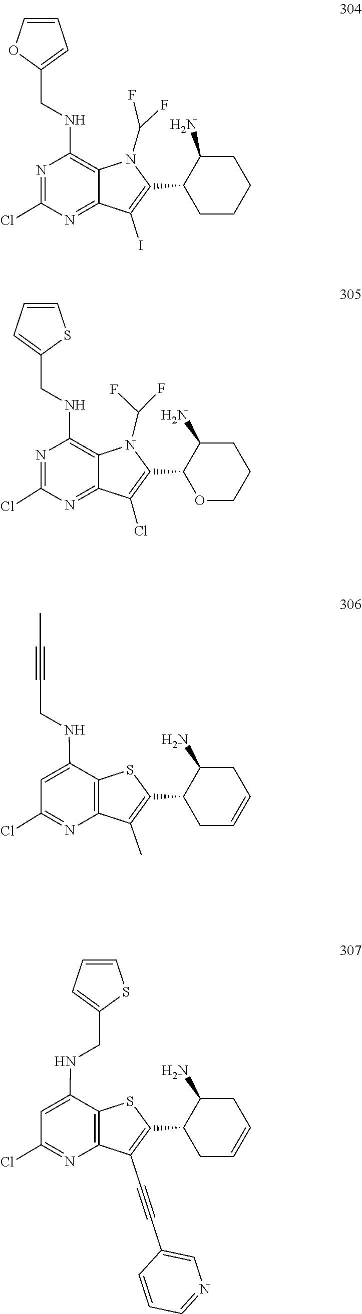 [SMILES: N[C@H]1CCCC[C@@H]1c1c(I)c2nc(Cl)nc(NCc3ccco3)c2n1C(F)F ; N[C@H]1CCCO[C@@H]1c1c(Cl)c2nc(Cl)nc(NCc3cccs3)c2n1C(F)F ; CC#CCNc1cc(Cl)nc2c(C)c([C@H]3CC=CC[C@@H]3N)sc12 ; N[C@H]1CC=CC[C@@H]1c1sc2c(NCc3cccs3)cc(Cl)nc2c1C#Cc1cccnc1]

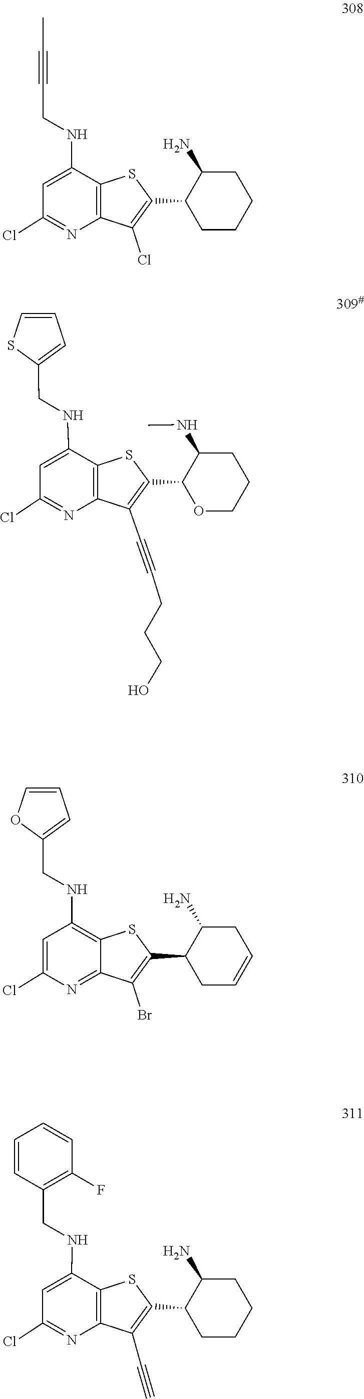 [SMILES: CC#CCNc1cc(Cl)nc2c(Cl)c([C@H]3CCCC[C@@H]3N)sc12 ; CN[C@H]1CCCO[C@@H]1c1sc2c(NCc3cccs3)cc(Cl)nc2c1C#CCCCO ; N[C@@H]1CC=CC[C@H]1c1sc2c(NCc3ccco3)cc(Cl)nc2c1Br ; C#Cc1c([C@H]2CCCC[C@@H]2N)sc2c(NCc3ccccc3F)cc(Cl)nc12]

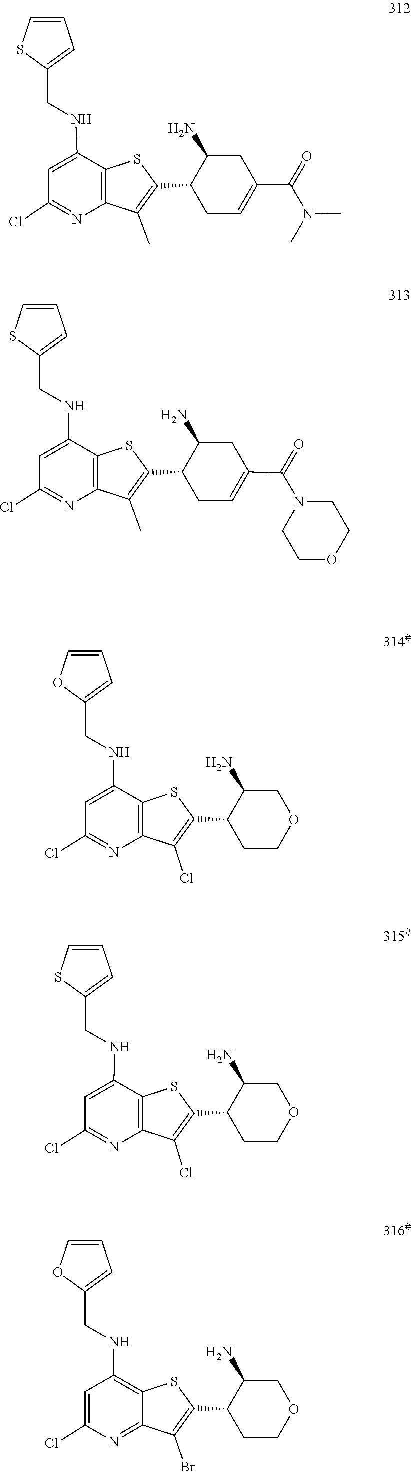 [SMILES: Cc1c([C@H]2CC=C(C(=O)N(C)C)C[C@@H]2N)sc2c(NCc3cccs3)cc(Cl)nc12 ; Cc1c([C@H]2CC=C(C(=O)N3CCOCC3)C[C@@H]2N)sc2c(NCc3cccs3)cc(Cl)nc12 ; N[C@H]1COCC[C@@H]1c1sc2c(NCc3ccco3)cc(Cl)nc2c1Cl ; N[C@H]1COCC[C@@H]1c1sc2c(NCc3cccs3)cc(Cl)nc2c1Cl ; N[C@H]1COCC[C@@H]1c1sc2c(NCc3ccco3)cc(Cl)nc2c1Br]

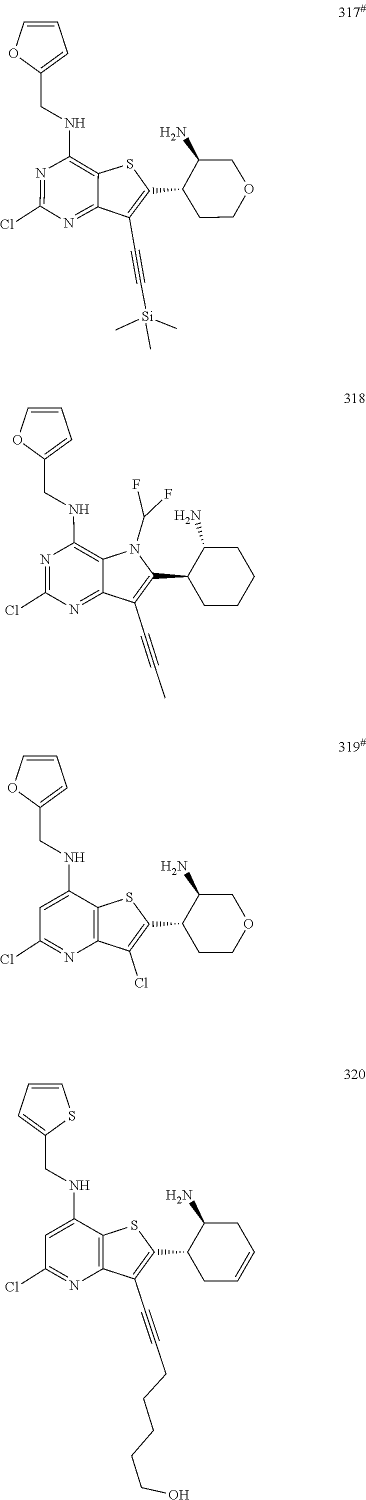 [SMILES: C[Si](C)(C)C#Cc1c([C@H]2CCOC[C@@H]2N)sc2c(NCc3ccco3)nc(Cl)nc12 ; CC#Cc1c([C@@H]2CCCC[C@H]2N)n(C(F)F)c2c(NCc3ccco3)nc(Cl)nc12 ; N[C@H]1COCC[C@@H]1c1sc2c(NCc3ccco3)cc(Cl)nc2c1Cl ; N[C@H]1CC=CC[C@@H]1c1sc2c(NCc3cccs3)cc(Cl)nc2c1C#CCCCCCO]

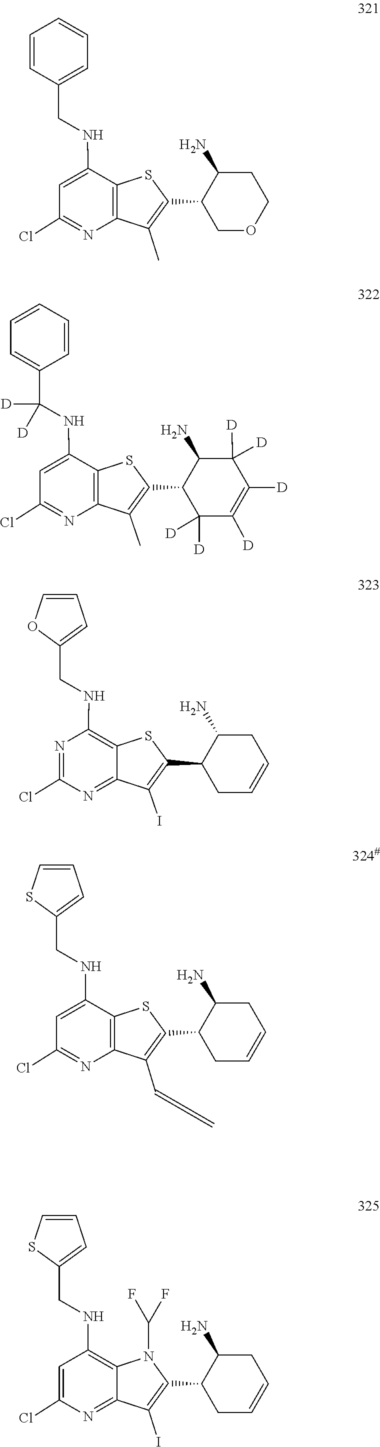 [SMILES: Cc1c([C@H]2COCC[C@@H]2N)sc2c(NCc3ccccc3)cc(Cl)nc12 ; [2H]C1=C([2H])C([2H])([2H])[C@H](c2sc3c(NC([2H])([2H])c4ccccc4)cc(Cl)nc3c2C)[C@@H](N)C1([2H])[2H] ; N[C@@H]1CC=CC[C@H]1c1sc2c(NCc3ccco3)nc(Cl)nc2c1I ; C=C=Cc1c([C@H]2CC=CC[C@@H]2N)sc2c(NCc3cccs3)cc(Cl)nc12 ; N[C@H]1CC=CC[C@@H]1c1c(I)c2nc(Cl)cc(NCc3cccs3)c2n1C(F)F]

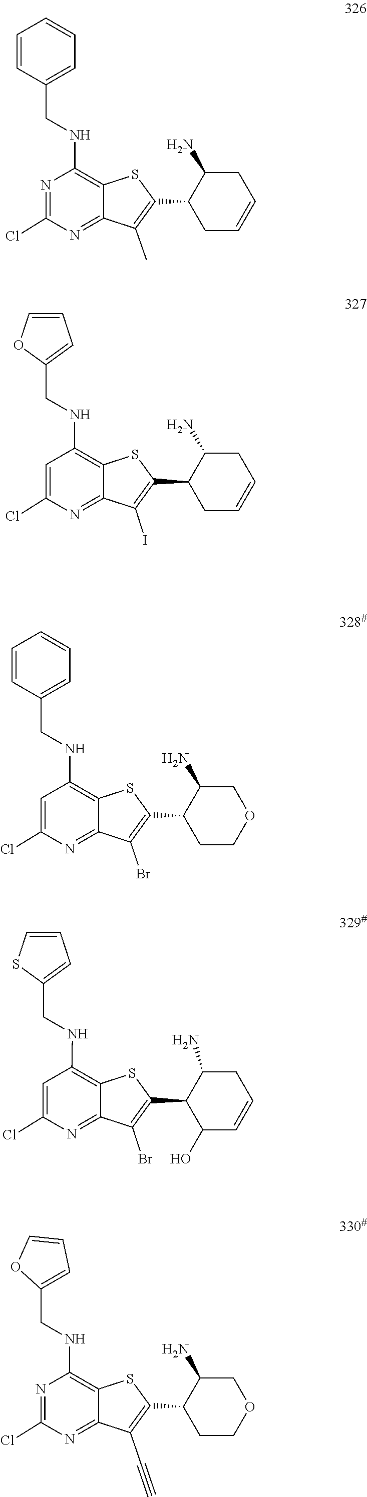 [SMILES: Cc1c([C@H]2CC=CC[C@@H]2N)sc2c(NCc3ccccc3)nc(Cl)nc12 ; N[C@@H]1CC=CC[C@H]1c1sc2c(NCc3ccco3)cc(Cl)nc2c1I ; N[C@H]1COCC[C@@H]1c1sc2c(NCc3ccccc3)cc(Cl)nc2c1Br ; N[C@@H]1CC=CC(O)[C@H]1c1sc2c(NCc3cccs3)cc(Cl)nc2c1Br ; C#Cc1c([C@H]2CCOC[C@@H]2N)sc2c(NCc3ccco3)nc(Cl)nc12]

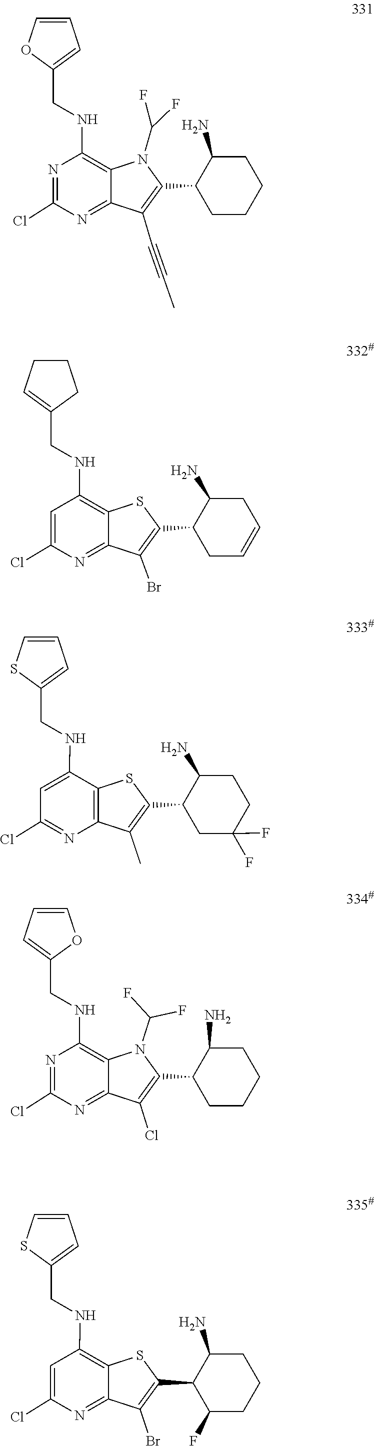 [SMILES: CC#Cc1c([C@H]2CCCC[C@@H]2N)n(C(F)F)c2c(NCc3ccco3)nc(Cl)nc12 ; N[C@H]1CC=CC[C@@H]1c1sc2c(NCC3=CCCC3)cc(Cl)nc2c1Br ; Cc1c([C@H]2CC(F)(F)CC[C@@H]2N)sc2c(NCc3cccs3)cc(Cl)nc12 ; N[C@H]1CCCC[C@@H]1c1c(Cl)c2nc(Cl)nc(NCc3ccco3)c2n1C(F)F ; N[C@H]1CCC[C@@H](F)[C@H]1c1sc2c(NCc3cccs3)cc(Cl)nc2c1Br]

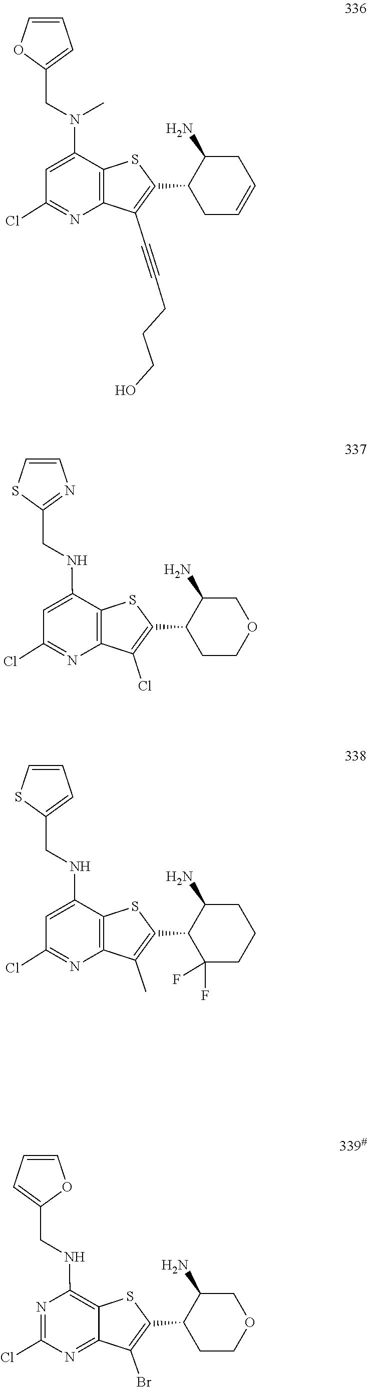 [SMILES: CN(Cc1ccco1)c1cc(Cl)nc2c(C#CCCCO)c([C@H]3CC=CC[C@@H]3N)sc12 ; N[C@H]1COCC[C@@H]1c1sc2c(NCc3nccs3)cc(Cl)nc2c1Cl ; Cc1c([C@@H]2[C@@H](N)CCCC2(F)F)sc2c(NCc3cccs3)cc(Cl)nc12 ; N[C@H]1COCC[C@@H]1c1sc2c(NCc3ccco3)nc(Cl)nc2c1Br]

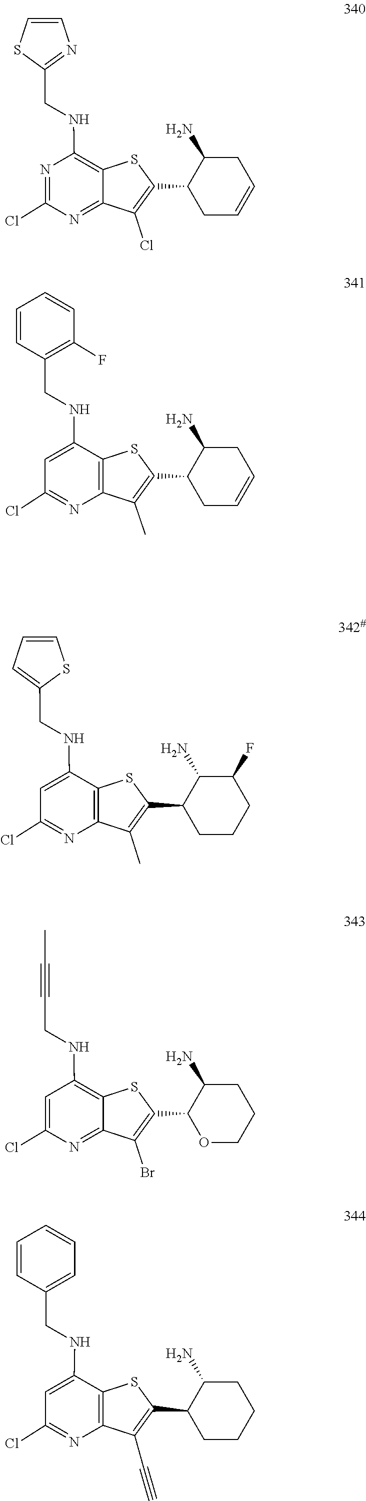 [SMILES: N[C@H]1CC=CC[C@@H]1c1sc2c(NCc3nccs3)nc(Cl)nc2c1Cl ; Cc1c([C@H]2CC=CC[C@@H]2N)sc2c(NCc3ccccc3F)cc(Cl)nc12 ; Cc1c([C@@H]2CCC[C@H](F)[C@H]2N)sc2c(NCc3cccs3)cc(Cl)nc12 ; CC#CCNc1cc(Cl)nc2c(Br)c([C@H]3OCCC[C@@H]3N)sc12 ; C#Cc1c([C@@H]2CCCC[C@H]2N)sc2c(NCc3ccccc3)cc(Cl)nc12]

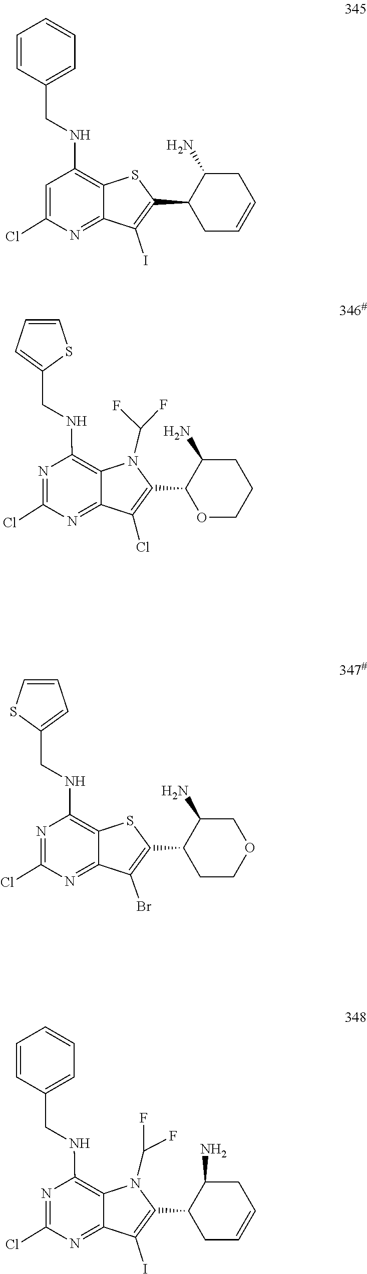 [SMILES: N[C@@H]1CC=CC[C@H]1c1sc2c(NCc3ccccc3)cc(Cl)nc2c1I ; N[C@H]1CCCO[C@@H]1c1c(Cl)c2nc(Cl)nc(NCc3cccs3)c2n1C(F)F ; N[C@H]1COCC[C@@H]1c1sc2c(NCc3cccs3)nc(Cl)nc2c1Br ; N[C@H]1CC=CC[C@@H]1c1c(I)c2nc(Cl)nc(NCc3ccccc3)c2n1C(F)F]

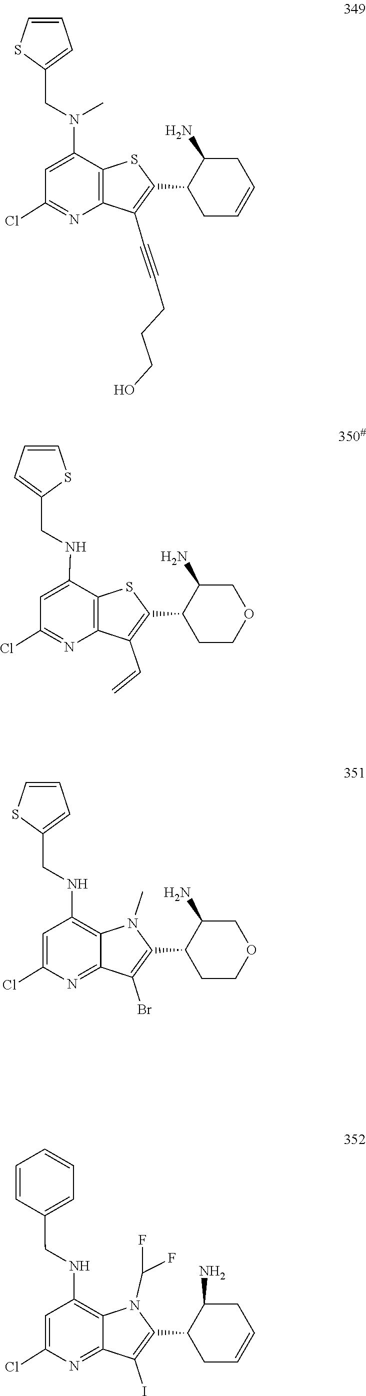

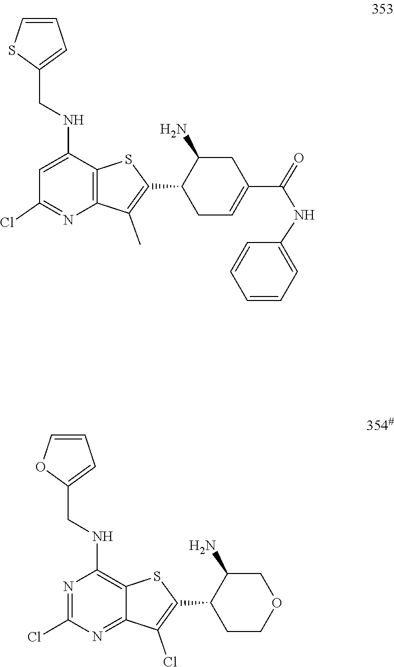 [SMILES: Cc1c([C@H]2CC=C(C(=O)Nc3ccccc3)C[C@@H]2N)sc2c(NCc3cccs3)cc(Cl)nc12 ; N[C@H]1COCC[C@@H]1c1sc2c(NCc3ccco3)nc(Cl)nc2c1Cl]

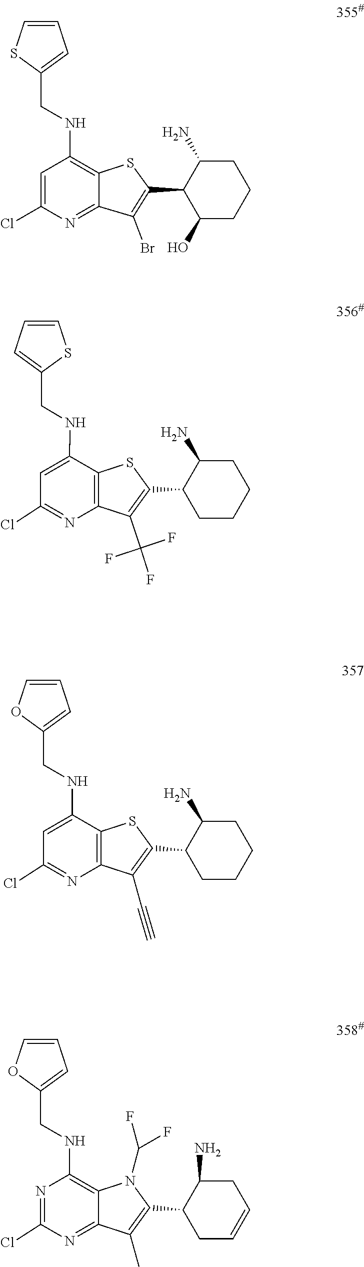 [SMILES: N[C@@H]1CCC[C@@H](O)[C@H]1c1sc2c(NCc3cccs3)cc(Cl)nc2c1Br ; N[C@H]1CCCC[C@@H]1c1sc2c(NCc3cccs3)cc(Cl)nc2c1C(F)(F)F ; C#Cc1c([C@H]2CCCC[C@@H]2N)sc2c(NCc3ccco3)cc(Cl)nc12 ; Cc1c([C@H]2CC=CC[C@@H]2N)n(C(F)F)c2c(NCc3ccco3)nc(Cl)nc12]

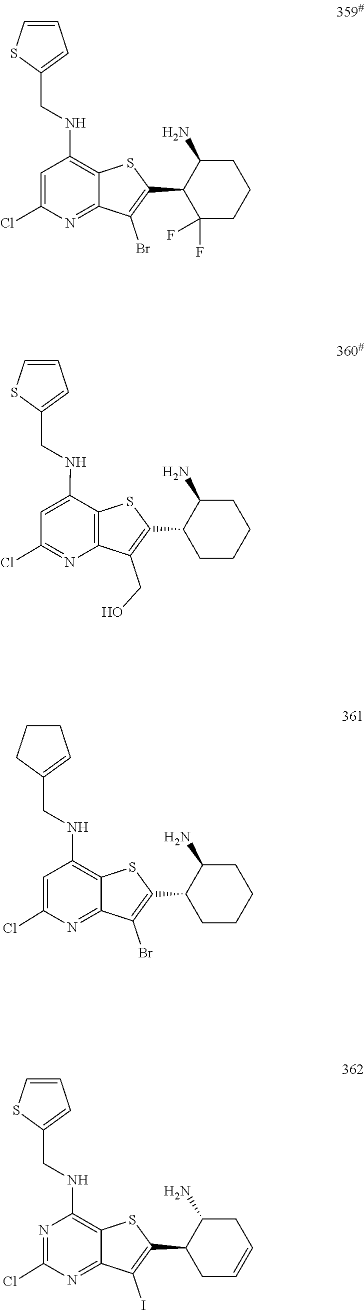 [SMILES: N[C@H]1CCCC(F)(F)[C@H]1c1sc2c(NCc3cccs3)cc(Cl)nc2c1Br ; N[C@H]1CCCC[C@@H]1c1sc2c(NCc3cccs3)cc(Cl)nc2c1CO ; N[C@H]1CCCC[C@@H]1c1sc2c(NCC3=CCCC3)cc(Cl)nc2c1Br ; N[C@@H]1CC=CC[C@H]1c1sc2c(NCc3cccs3)nc(Cl)nc2c1I]

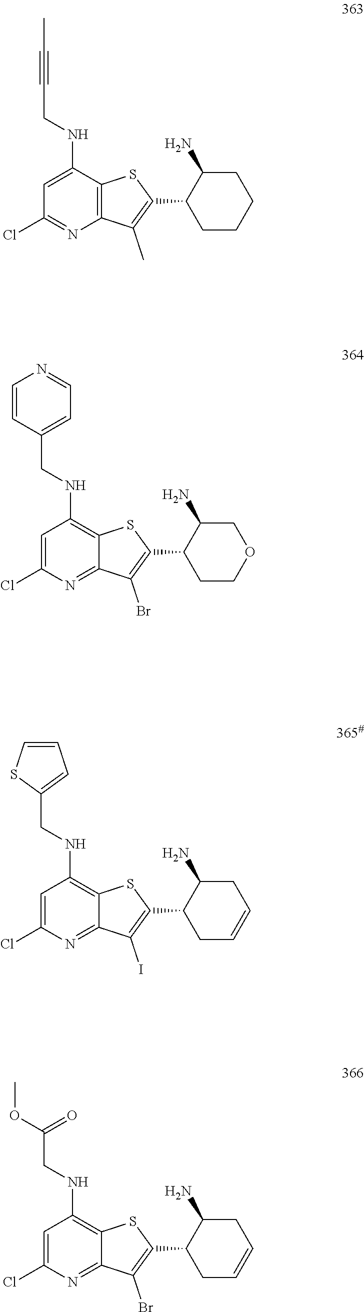 [SMILES: CC#CCNc1cc(Cl)nc2c(C)c([C@H]3CCCC[C@@H]3N)sc12 ; N[C@H]1COCC[C@@H]1c1sc2c(NCc3ccncc3)cc(Cl)nc2c1Br ; N[C@H]1CC=CC[C@@H]1c1sc2c(NCc3cccs3)cc(Cl)nc2c1I ; COC(=O)CNc1cc(Cl)nc2c(Br)c([C@H]3CC=CC[C@@H]3N)sc12]

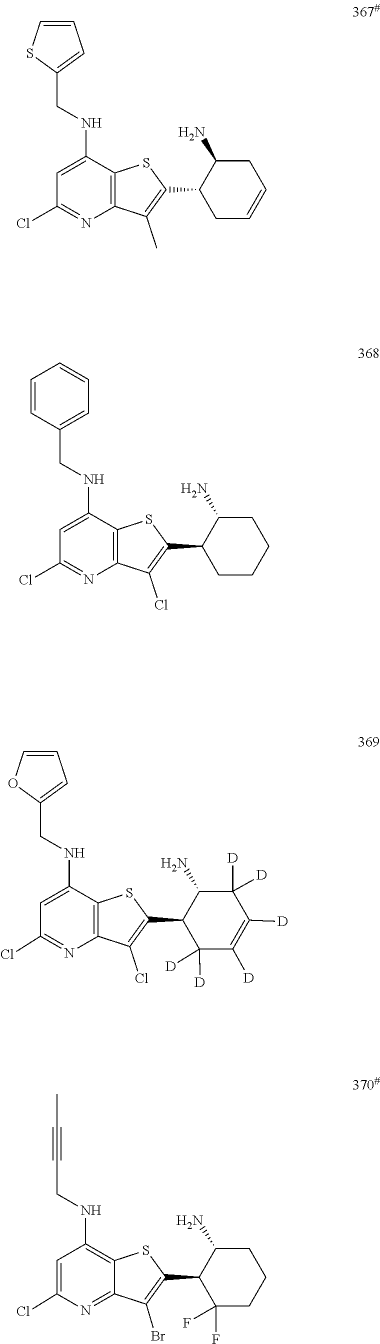 [SMILES: Cc1c([C@H]2CC=CC[C@@H]2N)sc2c(NCc3cccs3)cc(Cl)nc12 ; N[C@@H]1CCCC[C@H]1c1sc2c(NCc3ccccc3)cc(Cl)nc2c1Cl ; [2H]C1=C([2H])C([2H])([2H])[C@@H](c2sc3c(NCc4ccco4)cc(Cl)nc3c2Cl)[C@H](N)C1([2H])[2H] ; CC#CCNc1cc(Cl)nc2c(Br)c([C@H]3[C@H](N)CCCC3(F)F)sc12]

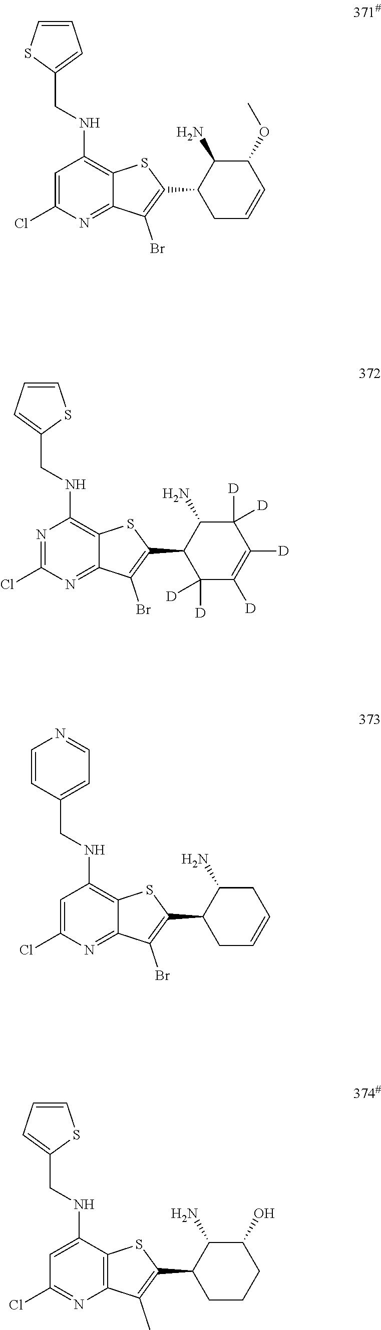 [SMILES: CO[C@@H]1C=CC[C@H](c2sc3c(NCc4cccs4)cc(Cl)nc3c2Br)[C@H]1N ; [2H]C1=C([2H])C([2H])([2H])[C@@H](c2sc3c(NCc4cccs4)nc(Cl)nc3c2Br)[C@H](N)C1([2H])[2H] ; N[C@@H]1CC=CC[C@H]1c1sc2c(NCc3ccncc3)cc(Cl)nc2c1Br ; Cc1c([C@@H]2CCC[C@@H](O)[C@H]2N)sc2c(NCc3cccs3)cc(Cl)nc12]

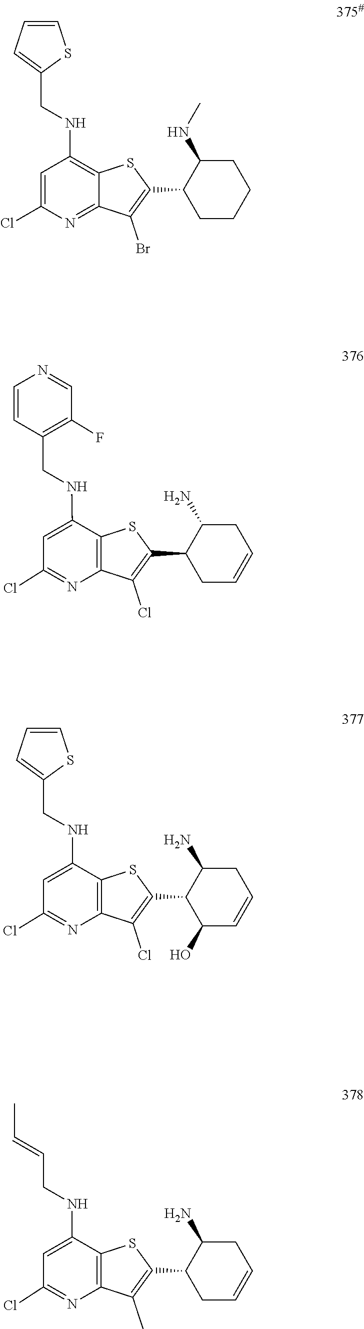 [SMILES: CN[C@H]1CCCC[C@@H]1c1sc2c(NCc3cccs3)cc(Cl)nc2c1Br ; N[C@@H]1CC=CC[C@H]1c1sc2c(NCc3ccncc3F)cc(Cl)nc2c1Cl ; N[C@H]1CC=C[C@@H](O)[C@@H]1c1sc2c(NCc3cccs3)cc(Cl)nc2c1Cl ; C/C=C/CNc1cc(Cl)nc2c(C)c([C@H]3CC=CC[C@@H]3N)sc12]

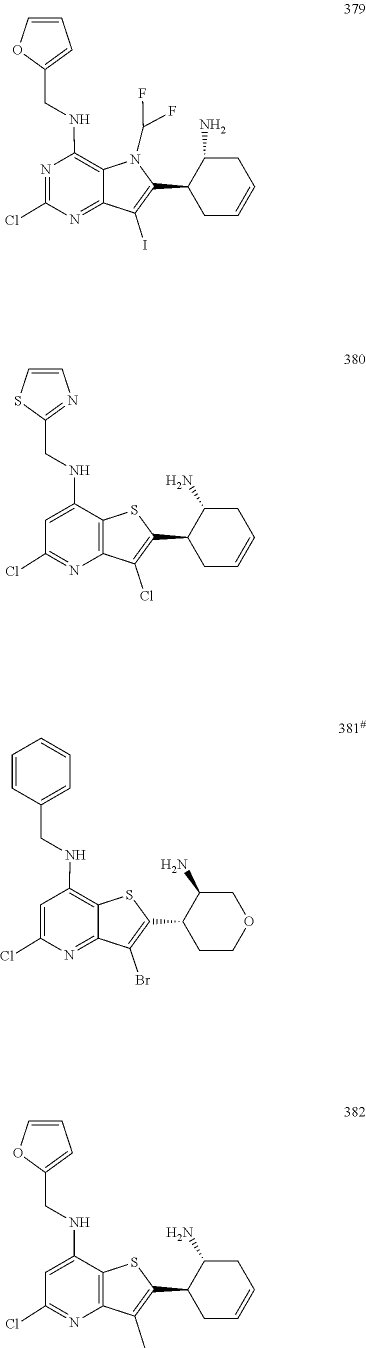 [SMILES: N[C@@H]1CC=CC[C@H]1c1c(I)c2nc(Cl)nc(NCc3ccco3)c2n1C(F)F ; N[C@@H]1CC=CC[C@H]1c1sc2c(NCc3nccs3)cc(Cl)nc2c1Cl ; N[C@H]1COCC[C@@H]1c1sc2c(NCc3ccccc3)cc(Cl)nc2c1Br ; Cc1c([C@@H]2CC=CC[C@H]2N)sc2c(NCc3ccco3)cc(Cl)nc12]

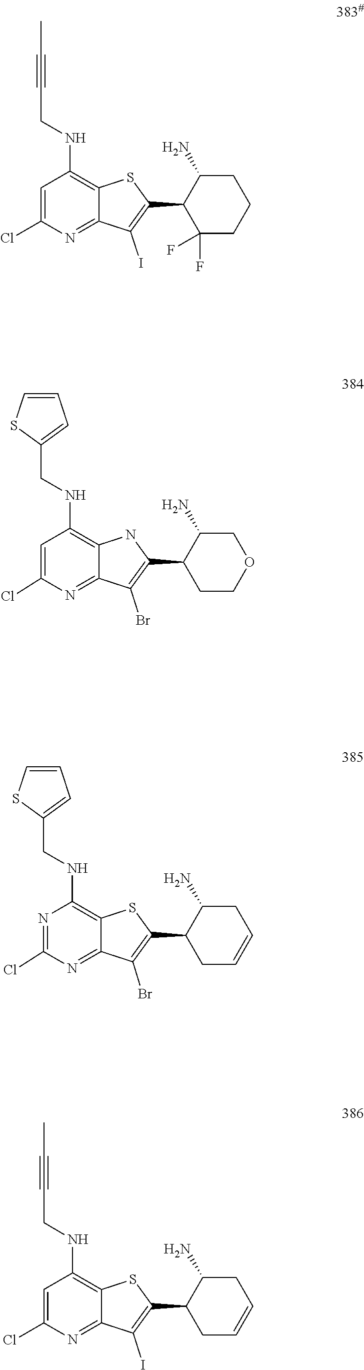

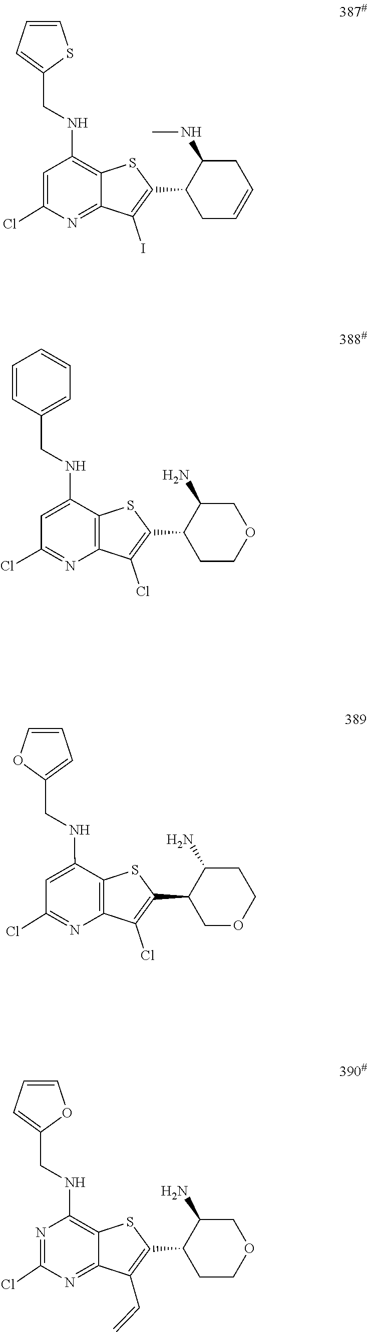 [SMILES: CN[C@H]1CC=CC[C@@H]1c1sc2c(NCc3cccs3)cc(Cl)nc2c1I ; N[C@H]1COCC[C@@H]1c1sc2c(NCc3ccccc3)cc(Cl)nc2c1Cl ; N[C@@H]1CCOC[C@H]1c1sc2c(NCc3ccco3)cc(Cl)nc2c1Cl ; C=Cc1c([C@H]2CCOC[C@@H]2N)sc2c(NCc3ccco3)nc(Cl)nc12]

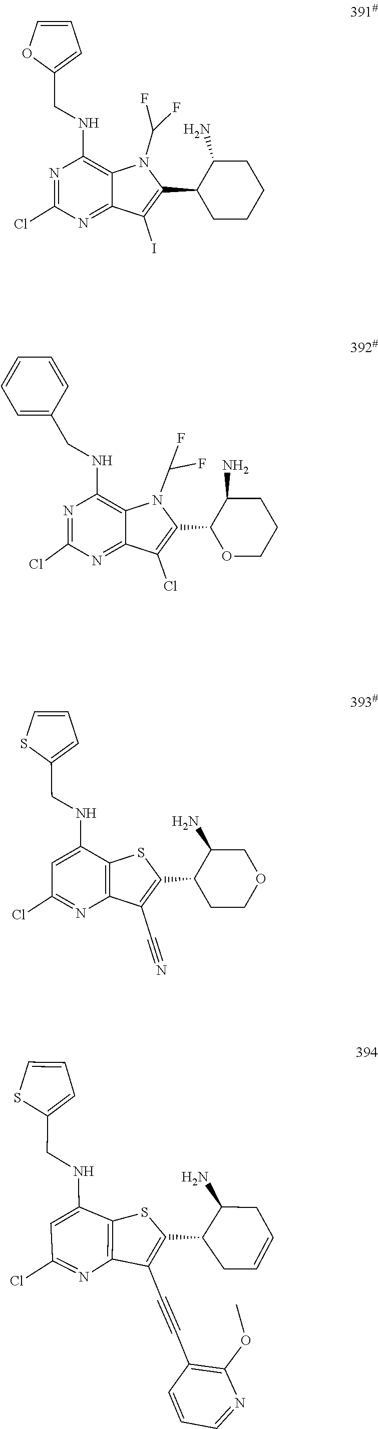 [SMILES: N[C@@H]1CCCC[C@H]1c1c(I)c2nc(Cl)nc(NCc3ccco3)c2n1C(F)F ; N[C@H]1CCCO[C@@H]1c1c(Cl)c2nc(Cl)nc(NCc3ccccc3)c2n1C(F)F ; N#Cc1c([C@H]2CCOC[C@@H]2N)sc2c(NCc3cccs3)cc(Cl)nc12 ; COc1ncccc1C#Cc1c([C@H]2CC=CC[C@@H]2N)sc2c(NCc3cccs3)cc(Cl)nc12]

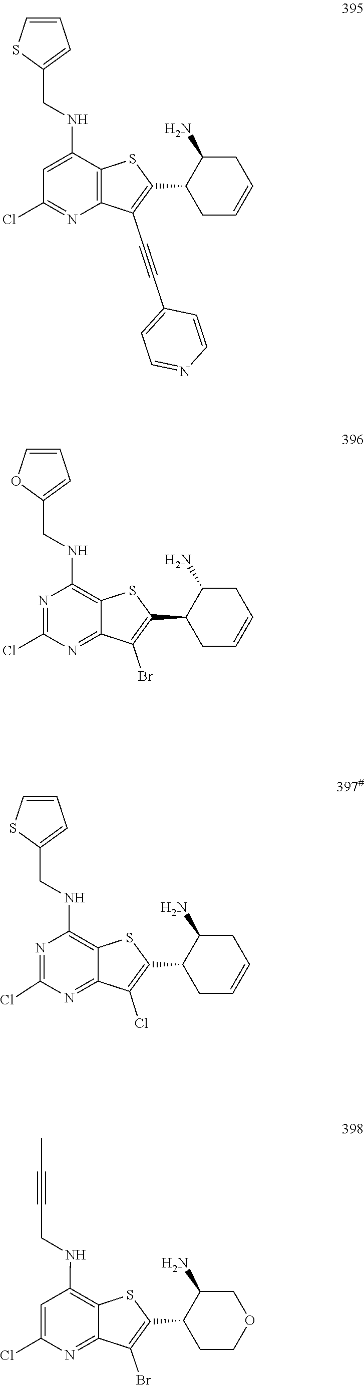 [SMILES: N[C@H]1CC=CC[C@@H]1c1sc2c(NCc3cccs3)cc(Cl)nc2c1C#Cc1ccncc1 ; N[C@@H]1CC=CC[C@H]1c1sc2c(NCc3ccco3)nc(Cl)nc2c1Br ; N[C@H]1CC=CC[C@@H]1c1sc2c(NCc3cccs3)nc(Cl)nc2c1Cl ; CC#CCNc1cc(Cl)nc2c(Br)c([C@H]3CCOC[C@@H]3N)sc12]

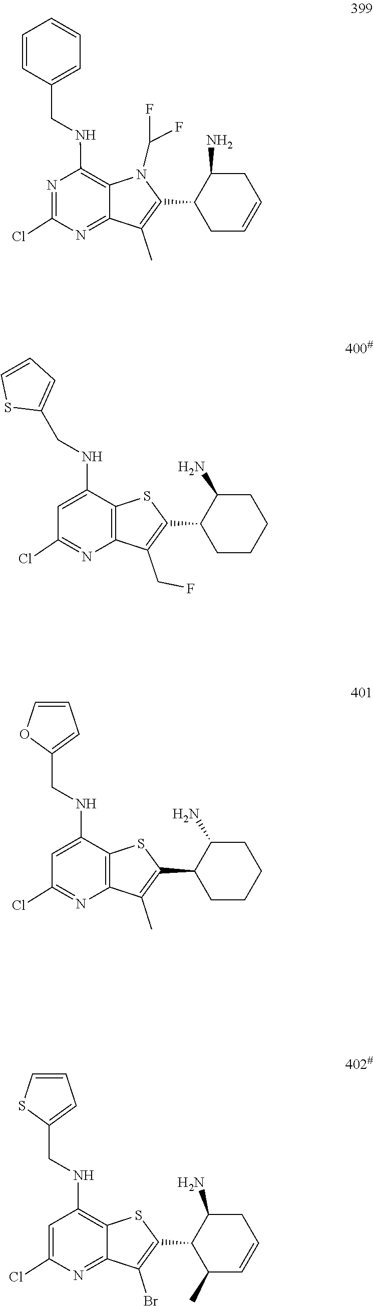 [SMILES: Cc1c([C@H]2CC=CC[C@@H]2N)n(C(F)F)c2c(NCc3ccccc3)nc(Cl)nc12 ; N[C@H]1CCCC[C@@H]1c1sc2c(NCc3cccs3)cc(Cl)nc2c1CF ; Cc1c([C@@H]2CCCC[C@H]2N)sc2c(NCc3ccco3)cc(Cl)nc12 ; C[C@@H]1C=CC[C@H](N)[C@H]1c1sc2c(NCc3cccs3)cc(Cl)nc2c1Br]

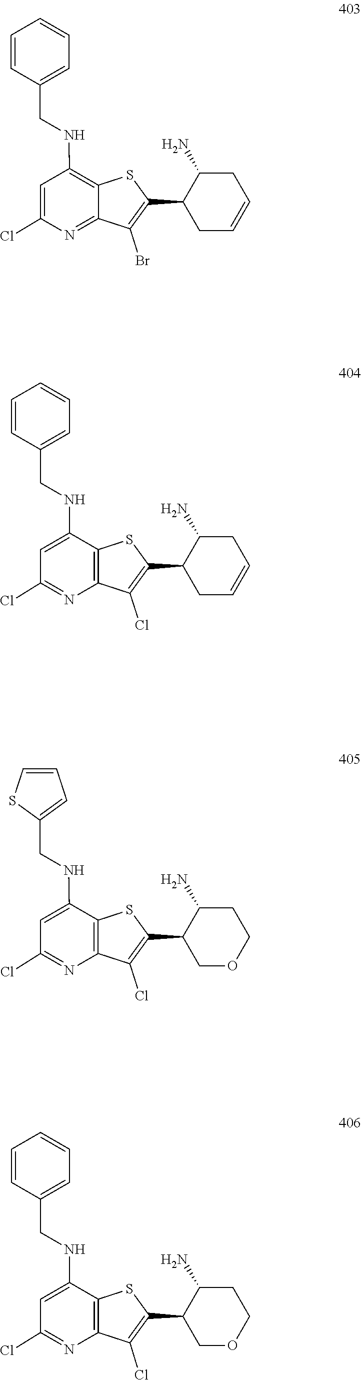 [SMILES: N[C@@H]1CC=CC[C@H]1c1sc2c(NCc3ccccc3)cc(Cl)nc2c1Br ; N[C@@H]1CC=CC[C@H]1c1sc2c(NCc3ccccc3)cc(Cl)nc2c1Cl ; N[C@@H]1CCOC[C@H]1c1sc2c(NCc3cccs3)cc(Cl)nc2c1Cl ; N[C@@H]1CCOC[C@H]1c1sc2c(NCc3ccccc3)cc(Cl)nc2c1Cl]

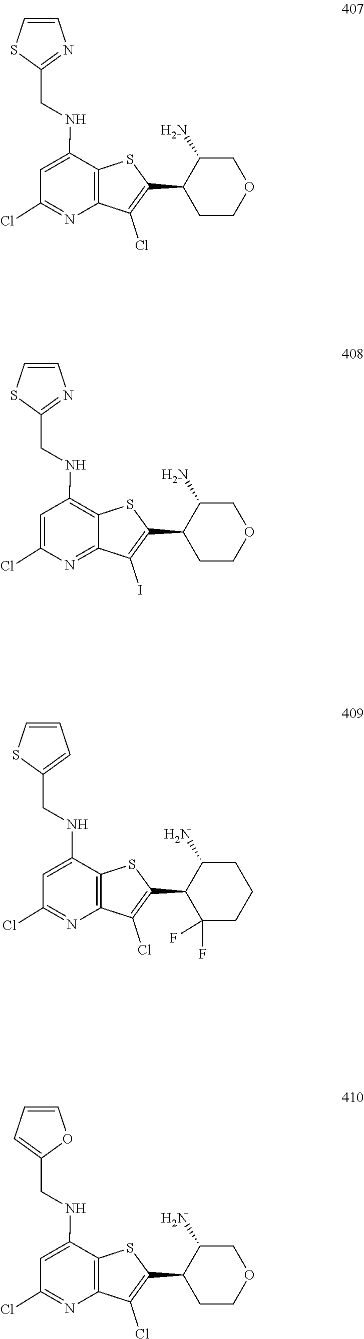 [SMILES: N[C@@H]1COCC[C@H]1c1sc2c(NCc3nccs3)cc(Cl)nc2c1Cl ; N[C@@H]1COCC[C@H]1c1sc2c(NCc3nccs3)cc(Cl)nc2c1I ; N[C@@H]1CCCC(F)(F)[C@H]1c1sc2c(NCc3cccs3)cc(Cl)nc2c1Cl ; N[C@@H]1COCC[C@H]1c1sc2c(NCc3ccco3)cc(Cl)nc2c1Cl]

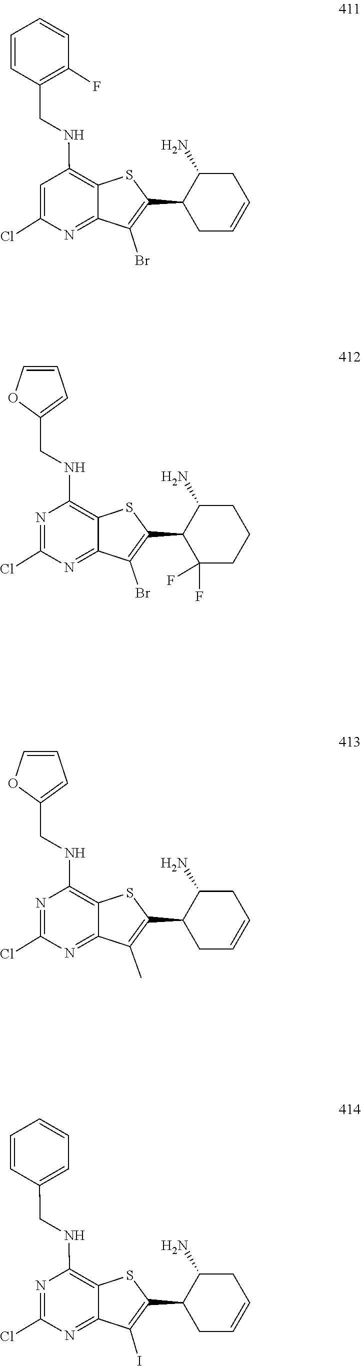 [SMILES: N[C@@H]1CC=CC[C@H]1c1sc2c(NCc3ccccc3F)cc(Cl)nc2c1Br ; N[C@@H]1CCCC(F)(F)[C@H]1c1sc2c(NCc3ccco3)nc(Cl)nc2c1Br ; Cc1c([C@@H]2CC=CC[C@H]2N)sc2c(NCc3ccco3)nc(Cl)nc12 ; N[C@@H]1CC=CC[C@H]1c1sc2c(NCc3ccccc3)nc(Cl)nc2c1I]

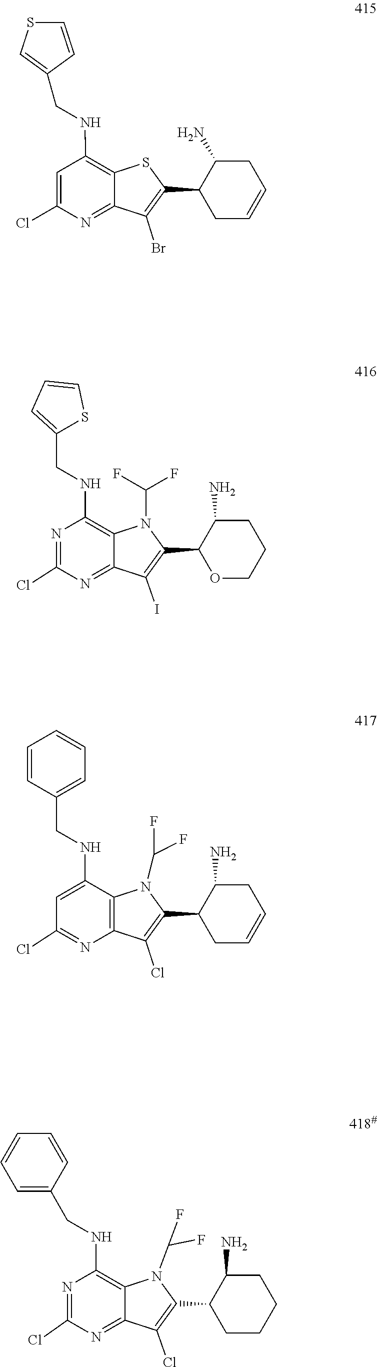 [SMILES: N[C@@H]1CC=CC[C@H]1c1sc2c(NCc3ccsc3)cc(Cl)nc2c1Br ; N[C@@H]1CCCO[C@H]1c1c(I)c2nc(Cl)nc(NCc3cccs3)c2n1C(F)F ; N[C@@H]1CC=CC[C@H]1c1c(Cl)c2nc(Cl)cc(NCc3ccccc3)c2n1C(F)F ; N[C@H]1CCCC[C@@H]1c1c(Cl)c2nc(Cl)nc(NCc3ccccc3)c2n1C(F)F]

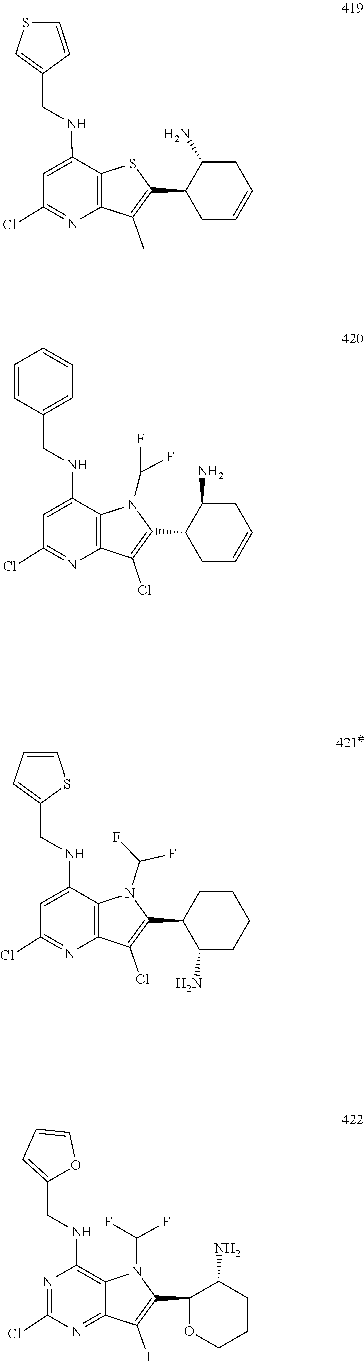 [SMILES: Cc1c([C@@H]2CC=CC[C@H]2N)sc2c(NCc3ccsc3)cc(Cl)nc12 ; N[C@H]1CC=CC[C@@H]1c1c(Cl)c2nc(Cl)cc(NCc3ccccc3)c2n1C(F)F ; N[C@H]1CCCC[C@@H]1c1c(Cl)c2nc(Cl)cc(NCc3cccs3)c2n1C(F)F ; N[C@@H]1CCCO[C@H]1c1c(I)c2nc(Cl)nc(NCc3ccco3)c2n1C(F)F]

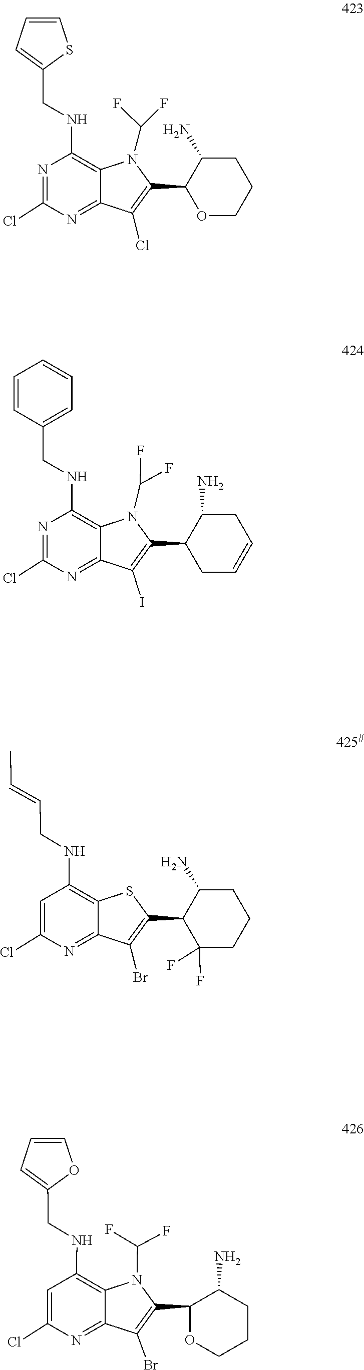 [SMILES: N[C@@H]1CCCO[C@H]1c1c(Cl)c2nc(Cl)nc(NCc3cccs3)c2n1C(F)F ; N[C@@H]1CC=CC[C@H]1c1c(I)c2nc(Cl)nc(NCc3ccccc3)c2n1C(F)F ; C/C=C/CNc1cc(Cl)nc2c(Br)c([C@H]3[C@H](N)CCCC3(F)F)sc12 ; N[C@@H]1CCCO[C@H]1c1c(Br)c2nc(Cl)cc(NCc3ccco3)c2n1C(F)F]

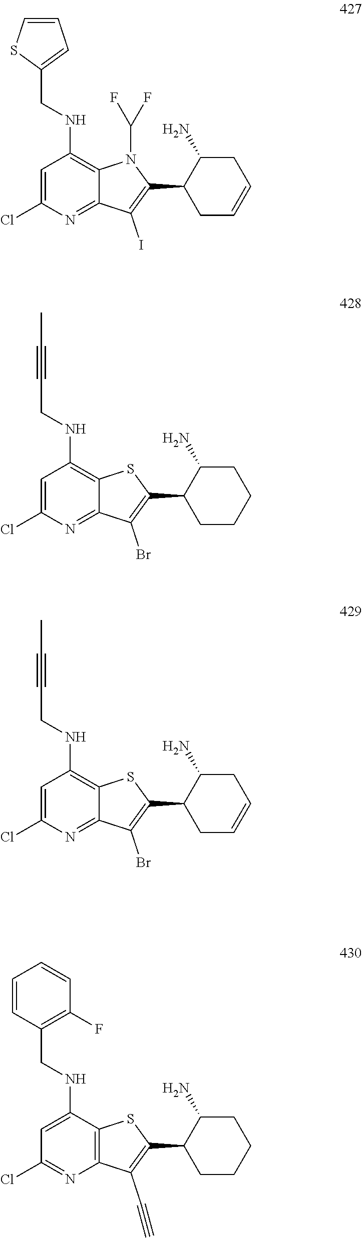 [SMILES: N[C@@H]1CC=CC[C@H]1c1c(I)c2nc(Cl)cc(NCc3cccs3)c2n1C(F)F ; CC#CCNc1cc(Cl)nc2c(Br)c([C@@H]3CCCC[C@H]3N)sc12 ; CC#CCNc1cc(Cl)nc2c(Br)c([C@@H]3CC=CC[C@H]3N)sc12 ; C#Cc1c([C@@H]2CCCC[C@H]2N)sc2c(NCc3ccccc3F)cc(Cl)nc12]

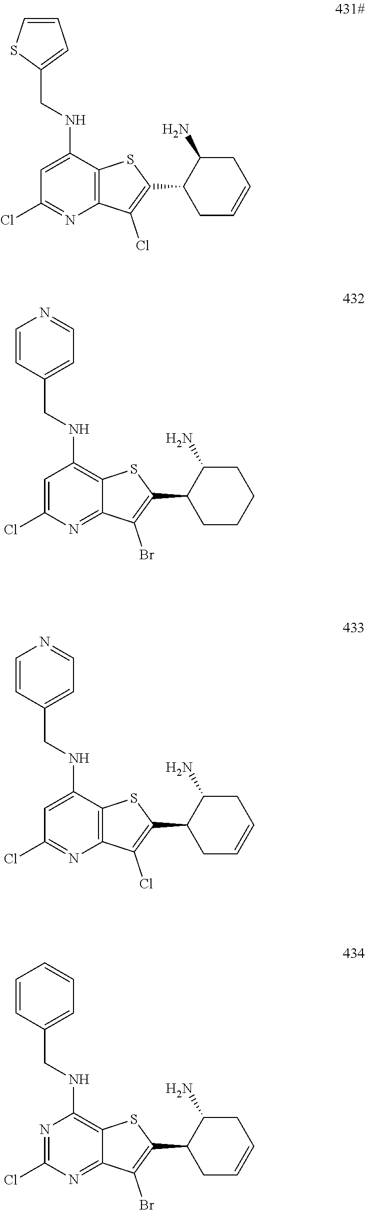 [SMILES: N[C@H]1CC=CC[C@@H]1c1sc2c(NCc3cccs3)cc(Cl)nc2c1Cl ; N[C@@H]1CCCC[C@H]1c1sc2c(NCc3ccncc3)cc(Cl)nc2c1Br ; N[C@@H]1CC=CC[C@H]1c1sc2c(NCc3ccncc3)cc(Cl)nc2c1Cl ; N[C@@H]1CC=CC[C@H]1c1sc2c(NCc3ccccc3)nc(Cl)nc2c1Br]

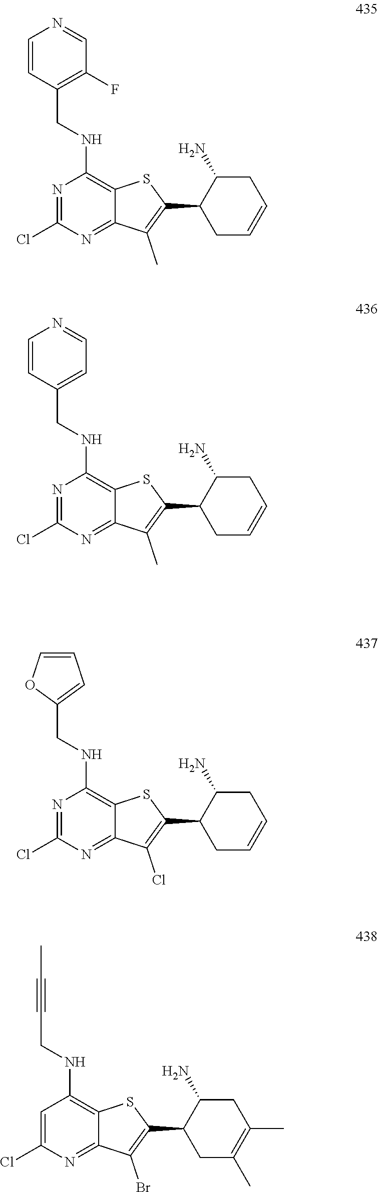 [SMILES: Cc1c([C@@H]2CC=CC[C@H]2N)sc2c(NCc3ccncc3F)nc(Cl)nc12 ; Cc1c([C@@H]2CC=CC[C@H]2N)sc2c(NCc3ccncc3)nc(Cl)nc12 ; N[C@@H]1CC=CC[C@H]1c1sc2c(NCc3ccco3)nc(Cl)nc2c1Cl ; CC#CCNc1cc(Cl)nc2c(Br)c([C@@H]3CC(C)=C(C)C[C@H]3N)sc12]

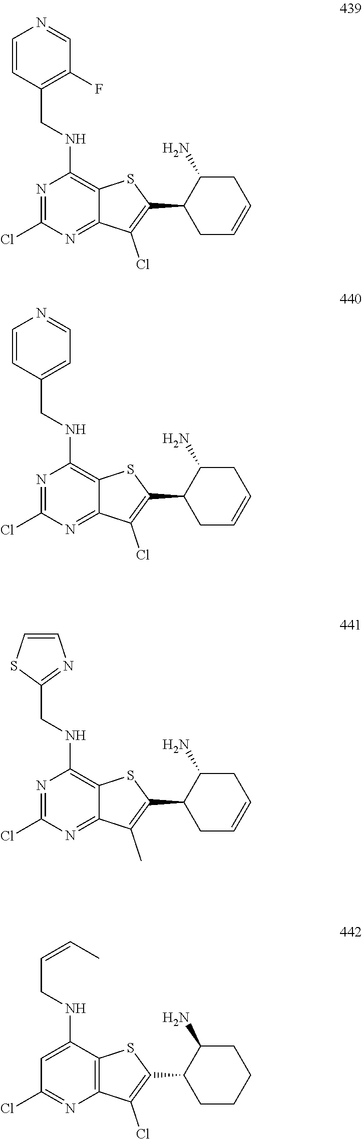 [SMILES: N[C@@H]1CC=CC[C@H]1c1sc2c(NCc3ccncc3F)nc(Cl)nc2c1Cl ; N[C@@H]1CC=CC[C@H]1c1sc2c(NCc3ccncc3)nc(Cl)nc2c1Cl ; Cc1c([C@@H]2CC=CC[C@H]2N)sc2c(NCc3nccs3)nc(Cl)nc12 ; C/C=C\CNc1cc(Cl)nc2c(Cl)c([C@H]3CCCC[C@@H]3N)sc12]

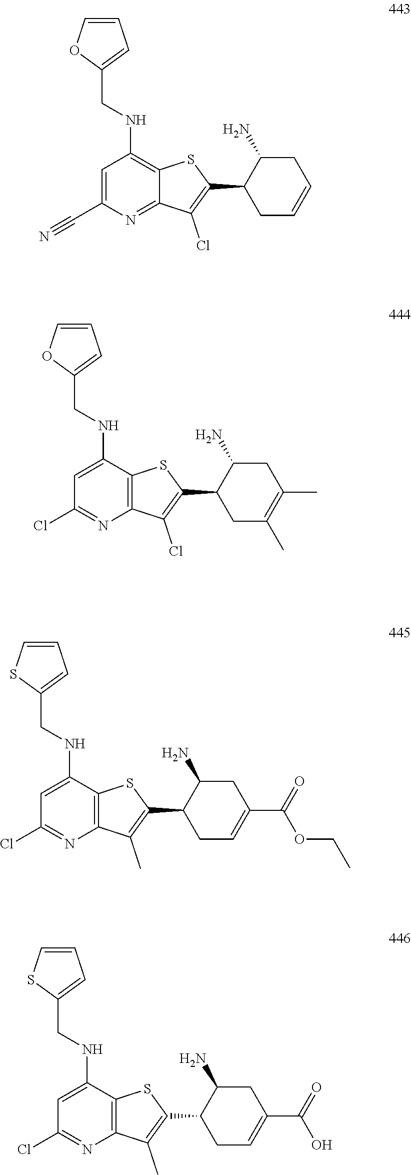 [SMILES: N#Cc1cc(NCc2ccco2)c2sc([C@@H]3CC=CC[C@H]3N)c(Cl)c2n1 ; CC1=C(C)C[C@@H](c2sc3c(NCc4ccco4)cc(Cl)nc3c2Cl)[C@H](N)C1 ; CCOC(=O)C1=CC[C@@H](c2sc3c(NCc4cccs4)cc(Cl)nc3c2C)[C@@H](N)C1 ; Cc1c([C@H]2CC=C(C(=O)O)C[C@@H]2N)sc2c(NCc3cccs3)cc(Cl)nc12]

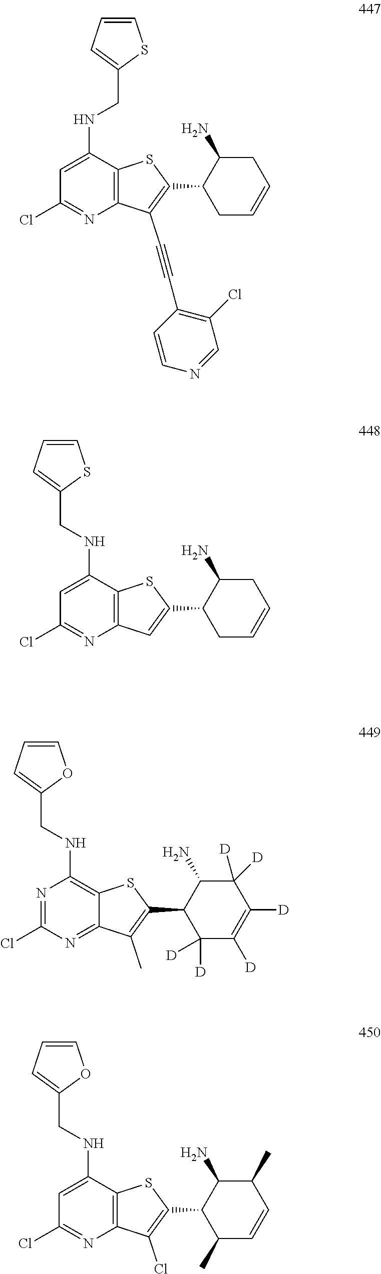 [SMILES: N[C@H]1CC=CC[C@@H]1c1sc2c(NCc3cccs3)cc(Cl)nc2c1C#Cc1ccncc1Cl ; N[C@H]1CC=CC[C@@H]1c1cc2nc(Cl)cc(NCc3cccs3)c2s1 ; [2H]C1=C([2H])C([2H])([2H])[C@@H](c2sc3c(NCc4ccco4)nc(Cl)nc3c2C)[C@H](N)C1([2H])[2H] ; C[C@@H]1C=C[C@H](C)[C@H](N)[C@H]1c1sc2c(NCc3ccco3)cc(Cl)nc2c1Cl]

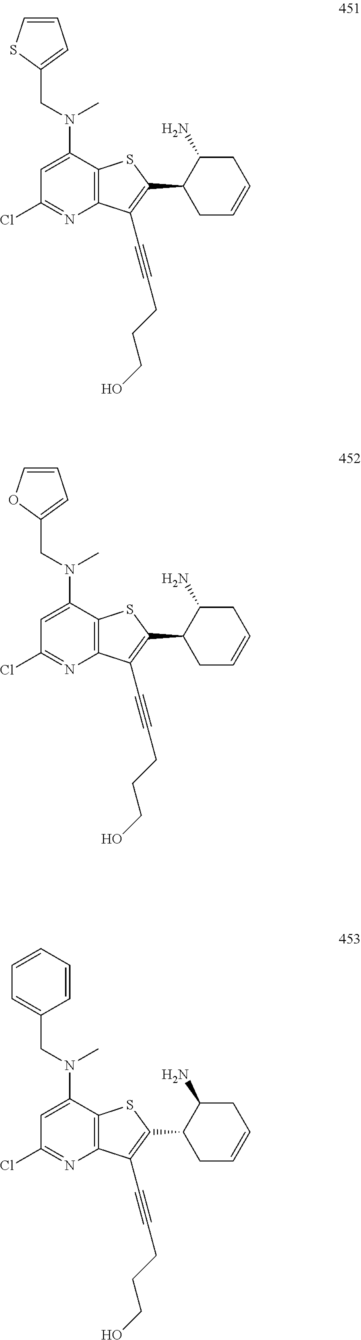 [SMILES: CN(Cc1cccs1)c1cc(Cl)nc2c(C#CCCCO)c([C@@H]3CC=CC[C@H]3N)sc12 ; CN(Cc1ccco1)c1cc(Cl)nc2c(C#CCCCO)c([C@@H]3CC=CC[C@H]3N)sc12 ; CN(Cc1ccccc1)c1cc(Cl)nc2c(C#CCCCO)c([C@H]3CC=CC[C@@H]3N)sc12]

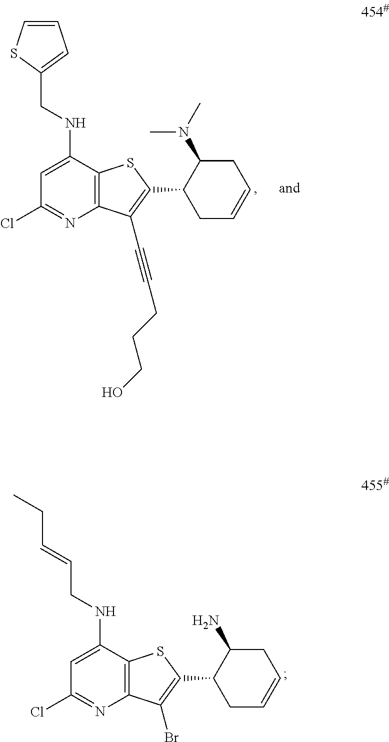 [SMILES: CN(C)[C@H]1CC=CC[C@@H]1c1sc2c(NCc3cccs3)cc(Cl)nc2c1C#CCCCO ; CC/C=C/CNc1cc(Cl)nc2c(Br)c([C@H]3CC=CC[C@@H]3N)sc12]

wherein the form of the compound is a pharmaceutically acceptable salt, hydrate, solvate, racemate, enantiomer, diastereomer, stereoisomer, tautomer or isotope enriched form thereof.

One aspect includes a compound of Formula (I) or a form thereof selected from

| Compound | Name |
|---|---|
| 1 | 2-((1S,2S)-2-aminocyclopentyl)-3,5-dichloro-N-(thiophen-2- |
| | ylmethyl)thieno[3,2-b]pyridin-7-amine |
| 2 | 2-((1R,2R)-2-aminocyclopentyl)-3,5-dichloro-N-(thiophen-2- |
| | ylmethyl)thieno[3,2-b]pyridin-7-amine |
| 3 | 2-((1S,2R)-2-aminocyclopentyl)-3,5-dichloro-N-(thiophen-2- |
| | ylmethyl)thieno[3,2-b]pyridin-7-amine |
| 4 | 2-((1R,2S)-2-aminocyclopentyl)-3,5-dichloro-N-(thiophen-2- |
| | ylmethyl)thieno[3,2-b]pyridin-7-amine |
| 5 | 2-((1R,2R)-2-aminocyclopentyl)-5-chloro-3-methyl-N-(thiophen-2- |
| | ylmethyl)thieno[3,2-b]pyridin-7-amine |
| 6 | 2-((1S,2S)-2-aminocyclopentyl)-5-chloro-3-methyl-N-(thiophen-2- |
| | ylmethyl)thieno[3,2-b]pyridin-7-amine |
| 7 | 2-((1R,2R)-2-aminocyclopentyl)-3-bromo-5-chloro-N-(thiophen-2- |
| | ylmethyl)thieno[3,2-b]pyridin-7-amine |
| 8 | 2-((1S,2S)-2-aminocyclopentyl)-3-bromo-5-chloro-N-(thiophen-2- |
| | ylmethyl)thieno[3,2-b]pyridin-7-amine |
| 9 | 2-((1S,2R)-2-aminocyclopentyl)-5-chloro-N-(thiophen-2- |
| | ylmethyl)thieno[3,2-b]pyridin-7-amine |
| 10 | 2-((1S,2S)-2-aminocyclopentyl)-5-chloro-N-(thiophen-2- |
| | ylmethyl)thieno[3,2-b]pyridin-7-amine |
| 11 | 2-((1S,2S)-2-aminocyclopentyl)-5-chloro-3-ethyl-N-(thiophen-2- |
| | ylmethyl)thieno[3,2-b]pyridin-7-amine |
| 12 | 2-((1R,2R)-2-aminocyclopentyl)-5-chloro-3-ethyl-N-(thiophen-2- |
| | ylmethyl)thieno[3,2-b]pyridin-7-amine |
| 13 | 2-((1R,2S)-2-aminocyclohexyl)-3,5-dichloro-N-(thiophen-2- |
| | ylmethyl)thieno[3,2-b]pyridin-7-amine |
| 14 | 2-((1S,2R)-2-aminocyclohexyl)-3,5-dichloro-N-(thiophen-2- |
| | ylmethyl)thieno[3,2-b]pyridin-7-amine |
| 15 | 2-((2S,3R)-3-aminotetrahydro-2H-pyran-2-yl)-3-bromo-5-chloro-N- |
| | (thiophen-2-ylmethyl)thieno[3,2-b]pyridin-7-amine |
| 16 | 2-((2R,3R)-3-aminotetrahydro-2H-pyran-2-yl)-3,5-dichloro-N-(thiophen-2- |
| | ylmethyl)thieno[3,2-b]pyridin-7-amine |
| 17 | 2-((2S,3R)-3-aminotetrahydro-2H-pyran-2-yl)-3,5-dichloro-N-(thiophen-2- |
| | ylmethyl)thieno[3,2-b]pyridin-7-amine |
| 18 | 2-[(2S,3S)-3-aminotetrahydropyran-2-yl]-3,5-dichloro-N-(2- |
| | thienylmethyl)thieno[3,2-b]pyridin-7-amine |
| 19 | 2-((2R,3S)-3-aminotetrahydro-2H-pyran-2-yl)-3,5-dichloro-N-(thiophen-2- |
| | ylmethyl)thieno[3,2-b]pyridin-7-amine |
| 20 | 3-bromo-5-chloro-2-[(2R,3S)-3-aminotetrahydropyran-2-yl]-N-(2- |
| | thienylmethyl)thieno[3,2-b]pyridin-7-amine and 3-bromo-5-chloro-2- |
| | [(2S,3R)-3-aminotetrahydropyran-2-yl]-N-(2-thienylmethyl)thieno[3,2- |
| | b]pyridin-7-amine |
| 21 | 2-((1S,2S)-2-aminocyclohexyl)-5-chloro-N-(thiophen-2- |
| | ylmethyl)thieno[3,2-b]pyridin-7-amine |
| 22 | 2-((1R,2S)-2-aminocyclohexyl)-5-chloro-N-(thiophen-2- |
| | ylmethyl)thieno[3,2-b]pyridin-7-amine |
| 23 | 2-((1R,2R)-2-aminocyclohexyl)-5-chloro-N-(thiophen-2- |
| | ylmethyl)thieno[3,2-b]pyridin-7-amine |
| 24 | 2-((2R,3R)-3-aminotetrahydro-2H-pyran-2-yl)-3,5-dichloro-N-(2- |
| | fluorobenzyl)thieno[3,2-b]pyridin-7-amine and 2-((2S,3S)-3-aminotetrahydro- |
| | 2H-pyran-2-yl)-3,5-dichloro-N-(2-fluorobenzyl)thieno[3,2-b]pyridin-7-amine |
| 25 | 2-((2R,3R)-3-aminotetrahydro-2H-pyran-2-yl)-5-chloro-N-(furan-2-ylmethyl)- |
| | 3-methylthieno[3,2-b]pyridin-7-amine and 2-((2S,3S)-3-aminotetrahydro-2H- |
| | pyran-2-yl)-5-chloro-N-(furan-2-ylmethyl)-3-methylthieno[3,2-b]pyridin-7- |
| | amine |
| 26 | 2-((2R,3R)-3-aminotetrahydro-2H-pyran-2-yl)-N-benzyl-5-chlorothieno[3,2- |
| | b]pyridin-7-amine and 2-((2S,3S)-3-aminotetrahydro-2H-pyran-2-yl)-N- |
| | benzyl-5-chlorothieno[3,2-b]pyridin-7-amine |
| 27 | 2-(3-aminotetrahydro-2H-pyran-2-yl)-N-benzyl-3-bromo-5-chlorothieno[3,2- |
| | b]pyridin-7-amine |
| 28 | 2-((2R,3R)-3-aminotetrahydro-2H-pyran-2-yl)-5-chloro-N-(2- |
| | fluorobenzyl)thieno[3,2-b]pyridin-7-amine and 2-((2S,3S)-3-aminotetrahydro- |
| | 2H-pyran-2-yl)-5-chloro-N-(2-fluorobenzyl)thieno[3,2-b]pyridin-7-amine |
| 29 | 2-((2R,3R)-3-aminotetrahydro-2H-pyran-2-yl)-3-bromo-5-chloro-N-(2- |
| | fluorobenzyl)thieno[3,2-b]pyridin-7-amine and 2-((2S,3S)-3-aminotetrahydro- |
| | 2H-pyran-2-yl)-3-bromo-5-chloro-N-(2-fluorobenzyl)thieno[3,2-b]pyridin-7- |
| | amine |
| 30 | 2-((1R,2S)-2-amino-4,4-difluorocyclohexyl)-3-bromo-5-chloro-N-(thiophen- |
| | 2-ylmethyl)thieno[3,2-b]pyridin-7-amine and 2-((1S,2R)-2-amino-4,4- |
| | difluorocyclohexyl)-3-bromo-5-chloro-N-(thiophen-2-ylmethyl)thieno[3,2- |
| | b]pyridin-7-amine |
| 31 | (1R,2S)-2-amino-1-(3,5-dichloro-7-((thiophen-2-ylmethyl)amino)thieno[3,2- |
| | b]pyridin-2-yl)cyclohexan-1-ol |
| 32 | 2-((1R,2S)-2-amino-1-fluorocyclohexyl)-3,5-dichloro-N-(thiophen-2- |
| | ylmethyl)thieno[3,2-b]pyridin-7-amine |
| 33 | 2-((2S,3S)-3-aminotetrahydro-2H-pyran-2-yl)-5-chloro-N-(2-fluorobenzyl)-3- |
| | methylthieno[3,2-b]pyridin-7-amine |
| 34 | 2-((1S,2S)-2-aminocyclohexyl)-3,5-dichloro-N-(thiophen-2- |
| | ylmethyl)thieno[3,2-b]pyridin-7-amine or 2-((1R,2R)-2-aminocyclohexyl)- |
| | 3,5-dichloro-N-(thiophen-2-ylmethyl)thieno[3,2-b]pyridine-7-amine |
| 35 | 2-((1R,2R)-2-aminocyclohexyl)-5-chloro-3-methyl-N-(thiophen-2- |
| | ylmethyl)thieno[3,2-b]pyridin-7-amine or 2-((1S,2S)-2-aminocyclohexyl)-5- |
| | chloro-3-methyl-N-(thiophen-2-ylmethyl)thieno[3,2-b]pyridine-7-amine |
| 36 | 2-((1R,2R)-2-aminocyclohexyl)-3,5-dichloro-N-(thiophen-2- |
| | ylmethyl)thieno[3,2-b]pyridin-7-amine or 2-((1S,2S)-2-aminocyclohexyl)-3,5- |
| | dichloro-N-(thiophen-2-ylmethyl)thieno[3,2-b]pyridine-7-amine |
| 37 | 2-((1R,2S)-2-amino-1-fluorocyclohexyl)-N-benzyl-3-bromo-5- |
| | chlorothieno[3,2-b]pyridin-7-amine |
| 38 | 2-((1R,2S)-2-amino-1-fluorocyclohexyl)-3,5-dichloro-N-(furan-2- |
| | ylmethyl)thieno[3,2-b]pyridin-7-amine |
| 39 | 2-((1R,2R)-2-aminocyclohexyl)-5-chloro-3-ethyl-N-(thiophen-2- |
| | ylmethyl)thieno[3,2-b]pyridin-7-amine or 2-((1S,2S)-2-aminocyclohexyl)-5- |
| | chloro-3-ethyl-N-(thiophen-2-ylmethyl)thieno[3,2-b]pyridine-7-amine |
| 40 | 2-((1S,2S)-2-aminocyclohexyl)-5-chloro-3-ethyl-N-(thiophen-2- |
| | ylmethyl)thieno[3,2-b]pyridin-7-amine or 2-((1R,2R)-2-aminocyclohexyl)-5- |
| | chloro-3-ethyl-N-(thiophen-2-ylmethyl)thieno[3,2-b]pyridin-7-amine |
| 41 | 2-((1R,2S)-2-amino-1-fluorocyclohexyl)-5-chloro-N-(furan-2-ylmethyl)-3- |
| | methylthieno[3,2-b]pyridin-7-amine |
| 42 | 6-((2R,3R)-3-aminotetrahydro-2H-pyran-2-yl)-7-bromo-2-chloro-N-(furan-2- |
| | ylmethyl)thieno[3,2-d]pyrimidin-4-amine or 6-((2S,3S)-3-aminotetrahydro- |
| | 2H-pyran-2-yl)-7-bromo-2-chloro-N-(furan-2-ylmethyl)thieno[3,2- |
| | d]pyrimidin-4-amine |
| 43 | 6-((1R,2R)-2-aminocyclohexyl)-N-benzyl-2-chloro-7-methylthieno[3,2- |
| | d]pyrimidin-4-amine or 6-((1S,2S)-2-aminocyclohexyl)-N-benzyl-2-chloro-7- |
| | methylthieno[3,2-d]pyrimidin-4-amine |
| 44 | 2-((2R,3R)-3-aminotetrahydro-2H-pyran-2-yl)-5-chloro-3-methyl-N- |
| | (thiophen-2-ylmethyl)thieno[3,2-b]pyridin-7-amine or 2-((2S,3S)-3- |
| | aminotetrahydro-2H-pyran-2-yl)-5-chloro-3-methyl-N-(thiophen-2- |
| | ylmethyl)thieno[3,2-b]pyridin-7-amine |
| 45 | 2-((2R,3R)-3-aminotetrahydro-2H-pyran-2-yl)-5-chloro-3-methyl-N- |
| | (thiophen-2-ylmethyl)thieno[3,2-b]pyridin-7-amine or 2-((2S,3S)-3- |
| | aminotetrahydro-2H-pyran-2-yl)-5-chloro-3-methyl-N-(thiophen-2- |
| | ylmethyl)thieno[3,2-b]pyridin-7-amine |
| 46 | 2-((2R,3S)-3-aminopiperidin-2-yl)-3-bromo-5-chloro-N-(thiophen-2- |
| | ylmethyl)thieno[3,2-b]pyridin-7-amine and 2-((2S,3R)-3-aminopiperidin-2- |
| | yl)-3-bromo-5-chloro-N-(thiophen-2-ylmethyl)thieno[3,2-b]pyridin-7-amine |
| 47 | 2-((2R,3R)-3-aminopiperidin-2-yl)-3-bromo-5-chloro-N-(thiophen-2- |
| | ylmethyl)thieno[3,2-b]pyridin-7-amine and 2-((2S,3S)-3-aminopiperidin-2- |
| | yl)-3-bromo-5-chloro-N-(thiophen-2-ylmethyl)thieno[3,2-b]pyridin-7-amine |
| 48 | 6-((1R,2R)-2-aminocyclohexyl)-N-benzyl-2-chloro-7-methylthieno[3,2- |
| | d]pyrimidin-4-amine or 6-((1S,2S)-2-aminocyclohexyl)-N-benzyl-2-chloro-7- |
| | methylthieno[3,2-d]pyrimidin-4-amine |
| 49 | 2-((2R,3R)-3-aminotetrahydro-2H-pyran-2-yl)-3-bromo-5-chloro-N- |
| | (thiophen-2-ylmethyl)thieno[3,2-b]pyridin-7-amine |
| 50 | 2-((1R,2S)-2-aminocyclohexyl)-5-chloro-3-methyl-N-(thiophen-2- |
| | ylmethyl)thieno[3,2-b]pyridin-7-amine |
| 51 | 2-((1S,2R)-2-aminocyclohexyl)-5-chloro-3-methyl-N-(thiophen-2- |
| | ylmethyl)thieno[3,2-b]pyridin-7-amine |
| 52 | 2-((1S,2R)-2-aminocyclohexyl)-3-bromo-5-chloro-N-(thiophen-2- |
| | ylmethyl)thieno[3,2-b]pyridin-7-amine |
| 53 | 2-((1R,2S)-2-aminocyclohexyl)-3-bromo-5-chloro-N-(thiophen-2- |
| | ylmethyl)thieno[3,2-b]pyridin-7-amine |
| 54 | 2-((1R,2R)-2-aminocyclohexyl)-3-bromo-5-chloro-N-(thiophen-2- |
| | ylmethyl)thieno[3,2-b]pyridin-7-amine |
| 55 | 2-((1S,2S)-2-aminocyclohexyl)-3-bromo-5-chloro-N-(thiophen-2- |
| | ylmethyl)thieno[3,2-b]pyridin-7-amine |
| 56 | 2-((2S,3S)-3-aminotetrahydro-2H-pyran-2-yl)-3-bromo-5-chloro-N-(thiophen- |
| | 2-ylmethyl)thieno[3,2-b]pyridin-7-amine |
| 57 | 2-((1S,2S)-2-aminocyclohexyl)-5-chloro-3-iodo-N-(thiophen-2- |
| | ylmethyl)thieno[3,2-b]pyridin-7-amine |
| 58 | 2-((1R,2R)-2-aminocyclohexyl)-5-chloro-3-iodo-N-(thiophen-2- |
| | ylmethyl)thieno[3,2-b]pyridin-7-amine |
| 59 | 6-((1R,2R)-2-aminocyclohexyl)-2-chloro-N-(furan-2-ylmethyl)-7- |
| | methylthieno[3,2-d]pyrimidin-4-amine |
| 60 | 6-((1S,2S)-2-aminocyclohexyl)-2-chloro-N-(furan-2-ylmethyl)-7- |
| | methylthieno[3,2-d]pyrimidin-4-amine |
| 61 | 6-((2R,3R)-3-aminotetrahydro-2H-pyran-2-yl)-2-chloro-N-(furan-2-ylmethyl)- |
| | 7-methylthieno[3,2-d]pyrimidin-4-amine |
| 62 | 6-((2S,3S)-3-aminotetrahydro-2H-pyran-2-yl)-2-chloro-N-(furan-2-ylmethyl)- |
| | 7-methylthieno[3,2-d]pyrimidin-4-amine |
| 63 | 6-((2S,3S)-3-aminotetrahydro-2H-pyran-2-yl)-N-benzyl-2-chloro-7- |
| | methylthieno[3,2-d]pyrimidin-4-amine |
| 64 | 6-((2R,3R)-3-aminotetrahydro-2H-pyran-2-yl)-N-benzyl-2-chloro-7- |
| | methylthieno[3,2-d]pyrimidin-4-amine |
| 65 | 6-[(1S,2S)-2-aminocyclohexyl]-N-benzyl-7-bromo-2-chloro-thieno[3,2- |
| | d]pyrimidin-4-amine |
| 66 | 6-((1R,2R)-2-aminocyclohexyl)-N-benzyl-7-bromo-2-chlorothieno[3,2- |
| | d]pyrimidin-4-amine |
| 67 | 6-((1R,2R)-2-aminocyclohexyl)-7-bromo-2-chloro-N-(furan-2- |
| | ylmethyl)thieno[3,2-d]pyrimidin-4-amine |
| 68 | 6-((1S,2S)-2-aminocyclohexyl)-7-bromo-2-chloro-N-(furan-2- |
| | ylmethyl)thieno[3,2-d]pyrimidin-4-amine |
| 69 | 6-((2S,3S)-3-aminotetrahydro-2H-pyran-2-yl)-N-benzyl-7-bromo-2- |
| | chlorothieno[3,2-d]pyrimidin-4-amine |
| 70 | 6-((2R,3R)-3-aminotetrahydro-2H-pyran-2-yl)-N-benzyl-7-bromo-2- |
| | chlorothieno[3,2-d]pyrimidin-4-amine |
| 71 | 2-((1S,2S)-2-aminocyclohexyl)-5-chloro-3-methyl-N-(thiophen-2- |
| | ylmethyl)thieno[3,2-b]pyridin-7-amine |
| 72 | 2-((1S,2R)-2-aminocycloheptyl)-3-bromo-5-chloro-N-(thiophen-2- |
| | ylmethyl)thieno[3,2-b]pyridin-7-amine |
| 73 | 2-((1R,2S)-2-aminocycloheptyl)-3-bromo-5-chloro-N-(thiophen-2- |
| | ylmethyl)thieno[3,2-b]pyridin-7-amine |
| 74 | 2-((1R,2R)-2-aminocycloheptyl)-3-bromo-5-chloro-N-(thiophen-2- |
| | ylmethyl)thieno[3,2-b]pyridin-7-amine |
| 75 | 2-((1S,2S)-2-aminocycloheptyl)-3-bromo-5-chloro-N-(thiophen-2- |
| | ylmethyl)thieno[3,2-b]pyridin-7-amine |
| 76 | 2-((1S,2R)-2-aminocycloheptyl)-3,5-dichloro-N-(thiophen-2- |
| | ylmethyl)thieno[3,2-b ]pyridin-7-amine |
| 77 | 2-((1R,2S)-2-aminocycloheptyl)-3,5-dichloro-N-(thiophen-2- |
| | ylmethyl)thieno[3,2-b]pyridin-7-amine |
| 78 | 2-((1R,2R)-2-aminocycloheptyl)-3,5-dichloro-N-(thiophen-2- |
| | ylmethyl)thieno[3,2-b]pyridin-7-amine |
| 79 | 2-((1S,2S)-2-aminocycloheptyl)-3,5-dichloro-N-(thiophen-2- |
| | ylmethyl)thieno[3,2-b]pyridin-7-amine |
| 80 | 2-((1R,2R)-2-aminocycloheptyl)-5-chloro-N-(thiophen-2-ylmethyl)thieno[3,2- |
| | b]pyridin-7-amine |
| 81 | 2-((1S,2S)-2-aminocycloheptyl)-5-chloro-3-methyl-N-(thiophen-2- |
| | ylmethyl)thieno[3,2-b]pyridin-7-amine |
| 82 | 2-((1R,2S)-2-aminocycloheptyl)-5-chloro-3-methyl-N-(thiophen-2- |
| | ylmethyl)thieno[3,2-b]pyridin-7-amine |
| 83 | 2-((1R,2R)-2-aminocycloheptyl)-5-chloro-3-methyl-N-(thiophen-2- |
| | ylmethyl)thieno[3,2-b]pyridin-7-amine |
| 84 | 2-((1S,2R)-2-aminocycloheptyl)-5-chloro-3-methyl-N-(thiophen-2- |
| | ylmethyl)thieno[3,2-b]pyridin-7-amine |
| 85 | 2-((2R,3R)-3-aminobicyclo[3.2.1]octan-2-yl)-3-bromo-5-chloro-N-(thiophen- |
| | 2-ylmethyl)thieno[3,2-b]pyridin-7-amine and 2-((2S,3S)-3- |
| | aminobicyclo[3.2.1]octan-2-yl)-3-bromo-5-chloro-N-(thiophen-2- |
| | ylmethyl)thieno[3,2-b]pyridin-7-amine |
| 86 | 2-((1R,2R)-2-aminocycloheptyl)-5-chloro-3-ethyl-N-(thiophen-2- |
| | ylmethyl)thieno[3,2-b]pyridin-7-amine or 2-((1S,2S)-2-aminocycloheptyl)-5- |
| | chloro-3-ethyl-N-(thiophen-2-ylmethyl)thieno[3,2-b]pyridin-7-amine |
| 87 | 2-((1S,2S)-2-aminocycloheptyl)-5-chloro-3-ethyl-N-(thiophen-2- |
| | ylmethyl)thieno[3,2-b]pyridin-7-amine or 2-((1R,2R)-2-aminocycloheptyl)-5- |
| | chloro-3-ethyl-N-(thiophen-2-ylmethyl)thieno[3,2-b]pyridin-7-amine |
| 88 | 2-((1S,2R)-2-aminocycloheptyl)-5-chloro-N-(thiophen-2-ylmethyl)thieno[3,2- |
| | b]pyridin-7-amine |
| 89 | 2-((1R,2S)-2-aminocycloheptyl)-5-chloro-N-(thiophen-2-ylmethyl)thieno[3,2- |
| | b]pyridin-7-amine |
| 90 | 2-((1S,2S)-2-aminocycloheptyl)-5-chloro-N-(thiophen-2-ylmethyl)thieno[3,2- |
| | b]pyridin-7-amine |
| 91 | 2-((1R,2S)-2-aminocyclooctyl)-3,5-dichloro-N-(thiophen-2- |
| | ylmethyl)thieno[3,2-b]pyridin-7-amine |
| 92 | 2-((1R,2R)-2-aminocyclooctyl)-5-chloro-3-methyl-N-(thiophen-2- |
| | ylmethyl)thieno[3,2-b]pyridin-7-amine |
| 93 | 2-((1S,2R)-2-aminocyclooctyl)-5-chloro-3-methyl-N-(thiophen-2- |
| | ylmethyl)thieno[3,2-b]pyridin-7-amine |
| 94 | 2-((1S,2S)-2-aminocyclooctyl)-5-chloro-3-methyl-N-(thiophen-2- |
| | ylmethyl)thieno[3,2-b]pyridin-7-amine |
| 95 | 2-((1R,2S)-2-aminocyclooctyl)-5-chloro-3-methyl-N-(thiophen-2- |
| | ylmethyl)thieno[3,2-b]pyridin-7-amine |
| 96 | 2-((1S,2S)-2-aminocyclooctyl)-3,5-dichloro-N-(thiophen-2- |
| | ylmethyl)thieno[3,2-b]pyridin-7-amine |
| 97 | 2-((1R,2R)-2-aminocyclooctyl)-3,5-dichloro-N-(thiophen-2- |
| | ylmethyl)thieno[3,2-b]pyridin-7-amine |
| 98 | 2-((1S,2R)-2-aminocyclooctyl)-3,5-dichloro-N-(thiophen-2- |
| | ylmethyl)thieno[3,2-b]pyridin-7-amine |
| 99 | 2-((1S,2S)-2-aminocyclooctyl)-3-bromo-5-chloro-N-(thiophen-2- |
| | ylmethyl)thieno[3,2-b]pyridin-7-amine |
| 100 | 2-((1R,2R)-2-aminocyclooctyl)-3-bromo-5-chloro-N-(thiophen-2- |
| | ylmethyl)thieno[3,2-b]pyridin-7-amine |
| 101 | 2-((1S,2R)-2-aminocyclooctyl)-3-bromo-5-chloro-N-(thi ophen-2- |
| | ylmethyl)thieno[3,2-b]pyridin-7-amine |
| 102 | 2-((1R,2S)-2-aminocyclooctyl)-3-bromo-5-chloro-N-(thi ophen-2- |
| | ylmethyl)thieno[3,2-b]pyridin-7-amine |
| 103 | 2-((1S,2S)-2-aminocyclooctyl)-5-chloro-N-(thiophen-2-ylmethyl)thieno[3,2- |
| | b]pyridin-7-amine |
| 104 | 2-((1R,2S)-2-aminocyclooctyl)-5-chloro-N-(thiophen-2-ylmethyl)thieno[3,2- |
| | b]pyridin-7-amine |
| 105 | 2-((1R,2R)-2-aminocyclooctyl)-5-chloro-N-(thiophen-2-ylmethyl)thieno[3,2- |
| | b]pyridin-7-amine |
| 106 | 2-((1S,2R)-2-aminocyclooctyl)-5-chloro-N-(thiophen-2-ylmethyl)thieno[3,2- |
| | b]pyridin-7-amine |
| 107 | 2-((1S,2S)-2-aminocyclopentyl)-N-benzyl-5-chloro-3-methylthieno[3,2- |
| | b]pyridin-7-amine |
| 108 | 2-((2S,3S)-3-aminotetrahydro-2H-pyran-2-yl)-N-benzyl-3-bromo-5- |
| | chlorothieno[3,2-b]pyridin-7-amine |
| 109 | 2-((2R,3R)-3-aminotetrahydro-2H-pyran-2-yl)-N-benzyl-5-chloro-3- |
| | methylthieno[3,2-b]pyridin-7-amine |
| 110 | 2-((2S,3S)-3-aminotetrahydro-2H-pyran-2-yl)-N-benzyl-3,5- |
| | dichlorothieno[3,2-b]pyridin-7-amine |
| 111 | 2-((1R,2S)-2-aminocyclopentyl)-5-chloro-N-(thiophen-2-ylmethyl)thieno[3,2- |
| | b]pyridin-7-amine |
| 112 | 2-((1R,2R)-2-aminocyclopentyl)-5-chloro-N-(thiophen-2-ylmethyl)thieno[3,2- |
| | b]pyridin-7-amine |
| 113 | 2-((2R,3R)-3-aminotetrahydro-2H-pyran-2-yl)-N-benzyl-3-bromo-5- |
| | chlorothieno[3,2-b]pyridin-7-amine |
| 114 | 2-((1R,2R)-2-aminocyclopentyl)-3,5-dichloro-N-(furan-2- |
| | ylmethyl)thieno[3,2-b]pyridin-7-amine |
| 115 | 2-((2S,3S)-3-aminotetrahydro-2H-pyran-2-yl)-3-bromo-5-chloro-N-(furan-2- |
| | ylmethyl)thieno[3,2-b]pyridin-7-amine |
| 116 | 2-((2S,3S)-3-aminotetrahydro-2H-pyran-2-yl)-3,5-dichloro-N-(furan-2- |
| | ylmethyl)thieno[3,2-b]pyridin-7-amine |
| 117 | 2-((1S,2S)-2-aminocyclopentyl)-3,5-dichloro-N-(furan-2-ylmethyl)thieno[3,2- |
| | b]pyridin-7-amine |
| 118 | 2-((2R,3R)-3-aminotetrahydro-2H-pyran-2-yl)-3-bromo-5-chloro-N-(furan-2- |
| | ylmethyl)thieno[3,2-b]pyridin-7-amine |
| 119 | 2-((2R,3R)-3-aminotetrahydro-2H-pyran-2-yl)-3,5-dichloro-N-(furan-2- |
| | ylmethyl)thieno[3,2-b]pyridin-7-amine |
| 120 | 2-((1S,2S)-2-aminocyclopentyl)-5-chloro-N-(furan-2-ylmethyl)-3- |
| | methylthieno[3,2-b]pyridin-7-amine |
| 121 | 2-((1R,2S)-2-amino-1-fluorocyclopentyl)-3,5-dichloro-N-(furan-2- |
| | ylmethyl)thieno[3,2-b]pyridin-7-amine |
| 122 | 2-((1S,2S)-2-aminocyclooctyl)-5-chloro-3-iodo-N-(thiophen-2- |
| | ylmethyl)thieno[3,2-b]pyridin-7-amine |
| 123 | 2-((1R,2R)-2-aminocyclooctyl)-5-chloro-3-iodo-N-(thiophen-2- |
| | ylmethyl)thieno[3,2-b]pyridin-7-amine |
| 124 | 2-((2R,3R)-3-amino-1-phenylpiperidin-2-yl)-3-bromo-5-chloro-N-(thiophen- |
| | 2-ylmethyl)thieno[3,2-b]pyridin-7-amine and 2-((2S,3S)-3-amino-1- |
| | phenylpiperidin-2-yl)-3-bromo-5-chloro-N-(thiophen-2-ylmethyl)thieno[3,2- |
| | b]pyridin-7-amine |
| 125 | 2-((2R,3S)-3-amino-1-phenylpiperidin-2-yl)-3-bromo-5-chloro-N-(thiophen- |
| | 2-ylmethyl)thieno[3,2-b]pyridin-7-amine and 2-((2S,3R)-3-amino-1- |
| | phenylpiperidin-2-yl)-3-bromo-5-chloro-N-(thiophen-2-ylmethyl)thieno[3,2- |
| | b]pyridin-7-amine |
| 126 | 2-((2R,3R)-3-aminotetrahydro-2H-pyran-2-yl)-N-benzyl-5-chloro-3- |
| | methylthieno[3,2-b]pyridin-7-amine |
| 127 | (1R,5S,6R)-6-amino-5-(3,5-dichloro-7-((thiophen-2- |
| | ylmethyl)amino)thieno[3,2-b]pyridin-2-yl)cyclohex-2-en-1-ol and (1S,5R,6S)- |
| | 6-amino-5-(3,5-dichloro-7-((thiophen-2-ylmethyl)amino)thieno[3,2-b]pyridin- |
| | 2-yl)cyclohex-2-en-1-ol |
| 128 | 6-((1R,2S)-2-amino-1-fluorocyclopentyl)-2-chloro-N-(furan-2-ylmethyl)-7- |
| | methylthieno[3,2-d]pyrimidin-4-amine |
| 129 | 2-((1S,2S)-2-aminocyclohexyl)-5-chloro-3-phenyl-N-(thiophen-2- |
| | ylmethyl)thieno[3,2-b]pyridin-7-amine |
| 130 | 2-((1S,2S)-2-aminocyclohexyl)-5-chloro-3-cyclopropyl-N-(thiophen-2- |
| | ylmethyl)thieno[3,2-b]pyridin-7-amine |
| 131 | 2-((1S,2S)-2-aminocyclopentyl)-5-chloro-3-phenyl-N-(thiophen-2- |
| | ylmethyl)thieno[3,2-b]pyridin-7-amine |
| 132 | 2-((2R,3R)-3-aminotetrahydro-2H-pyran-2-yl)-N-benzyl-5-chloro-3- |
| | iodothieno[3,2-b]pyridin-7-amine |
| 133 | 2-((2S,3S)-3-aminotetrahydro-2H-pyran-2-yl)-5-chloro-N-(furan-2-ylmethyl)- |
| | 3-methylthieno[3,2-b]pyridin-7-amine |
| 134 | ((2R,3R)-3-aminotetrahydro-2H-pyran-2-yl)-5-chloro-N-(furan-2-ylmethyl)-3- |
| | methylthieno[3,2-b]pyridin-7-amine |
| 135 | 2-((1R,2R)-2-aminocyclopentyl)-5-chloro-3-phenyl-N-(thiophen-2- |
| | ylmethyl)thieno[3,2-b]pyridin-7-amine |
| 136 | 2-((2S,3S)-3-aminotetrahydro-2H-pyran-2-yl)-N-benzyl-5-chloro-3- |
| | iodothieno[3,2-b]pyridin-7-amine |
| 137 | 2-((1R,2R)-2-aminocyclohexyl)-5-chloro-3-cyclopropyl-N-(thiophen-2- |
| | ylmethyl)thieno[3,2-b]pyridin-7-amine |
| 138 | 2-((1R,2R)-2-aminocyclohexyl)-5-chloro-3-cyclopropyl-N-(thiophen-2- |
| | ylmethyl)thieno[3,2-b]pyridin-7-amine |
| 139 | 2-((3R,4S)-3-aminotetrahydro-2H-pyran-4-yl)-3-bromo-5-chloro-N-(thiophen- |
| | 2-ylmethyl)thieno[3,2-b]pyridin-7-amine |
| 140 | 2-((3S,4R)-3-aminotetrahydro-2H-pyran-4-yl)-3-bromo-5-chloro-N-(thiophen- |
| | 2-ylmethyl)thieno[3,2-b]pyridin-7-amine |
| 141 | 2-((1S,2S)-2-aminocycloheptyl)-5-chloro-3-phenyl-N-(thiophen-2- |
| | ylmethyl)thieno[3,2-b]pyridin-7-amine |
| 142 | 2-((1R,2R)-2-aminocycloheptyl)-5-chloro-3-phenyl-N-(thiophen-2- |
| | ylmethyl)thieno[3,2-b]pyridin-7-amine |
| 143 | 6-((2S,3S)-3-aminotetrahydro-2H-pyran-2-yl)-7-bromo-2-chloro-N-(furan-2- |
| | ylmethyl)thieno[3,2-d]pyrimidin-4-amine |
| 144 | 2-((1S,6RS)-6-aminocyclohex-3-en-1-yl)-3-bromo-5-chloro-N-(thiophen-2- |
| | ylmethyl)thieno[3,2-b]pyridin-7-amine and 2-((1R,6SR)-6-aminocyclohex-3- |
| | en-1-yl)-3-bromo-5-chloro-N-(thiophen-2-ylmethyl)thieno[3,2-b]pyridin-7- |
| | amine |
| 145 | 2-((1R,2R)-2-aminocyclopentyl)-5-chloro-N-(furan-2-ylmethyl)-3- |
| | methylthieno[3,2-b]pyridin-7-amine |
| 146 | 2-((3R,4R)-3-aminotetrahydro-2H-pyran-4-yl)-3-bromo-5-chloro-N- |
| | (thiophen-2-ylmethyl)thieno[3,2-b]pyridin-7-amine and 2-((3S,4S)-3- |
| | aminotetrahydro-2H-pyran-4-yl)-3-bromo-5-chloro-N-(thiophen-2- |
| | ylmethyl)thieno[3,2-b]pyridin-7-amine |
| 147 | 2-((4R,5R)-5-aminoazepan-4-yl)-3-bromo-5-chloro-N-(thiophen-2- |
| | ylmethyl)thieno[3,2-b]pyridin-7-amine and 2-((4S,5S)-5-aminoazepan-4-yl)- |
| | 3-bromo-5-chloro-N-(thiophen-2-ylmethyl)thieno[3,2-b]pyridin-7-amine |
| 148 | 2-((4R,5S)-5-aminoazepan-4-yl)-3-bromo-5-chloro-N-(thiophen-2- |
| | ylmethyl)thieno[3,2-b]pyridin-7-amine and 2-((4S,5S)-5-aminoazepan-4-yl)- |
| | 3-bromo-5-chloro-N-(thiophen-2-ylmethyl)thieno[3,2-b]pyridin-7-amine |
| 149 | 2-((1R,2R)-2-amino-5,5-dimethylcyclohexyl)-3-bromo-5-chloro-N-(thiophen- |
| | 2-ylmethyl)thieno[3,2-b]pyridin-7-amine and 2-((1S,2S)-2-amino-5,5- |
| | dimethylcyclohexyl)-3-bromo-5-chloro-N-(thiophen-2-ylmethyl)thieno[3,2- |
| | b]pyridin-7-amine |
| 150 | 2-((2R,3S)-3-aminotetrahydrofuran-2-yl)-3-bromo-5-chloro-N-(thiophen-2- |
| | ylmethyl)thieno[3,2-b]pyridin-7-amine and 2-((2S,3R)-3- |
| | aminotetrahydrofuran-2-yl)-3-bromo-5-chloro-N-(thiophen-2- |
| | ylmethyl)thieno[3,2-b]pyridin-7-amine |
| 151 | 2-((2R,3R)-3-aminotetrahydrofuran-2-yl)-3-bromo-5-chloro-N-(thiophen-2- |
| | ylmethyl)thieno[3,2-b]pyridin-7-amine and 2-((2S,3S)-3- |
| | aminotetrahydrofuran-2-yl)-3-bromo-5-chloro-N-(thiophen-2- |
| | ylmethyl)thieno[3,2-b]pyridin-7-amine |
| 152 | 2-((1R,2R)-2-amino-4,4-dimethylcyclohexyl)-3-bromo-5-chloro-N-(thiophen- |
| | 2-ylmethyl)thieno[3,2-b]pyridin-7-amine and 2-((1S,2S)-2-amino-4,4- |
| | dimethylcyclohexyl)-3-bromo-5-chloro-N-(thiophen-2-ylmethyl)thieno[3,2- |
| | b]pyridin-7-amine |
| 153 | (1R,5S,6R)-6-amino-5-(3-bromo-5-chloro-7-((thiophen-2- |
| | ylmethyl)amino)thieno[3,2-b]pyridin-2-yl)cyclohex-2-en-1-ol and (1S,5R,6S)- |
| | 6-amino-5-(3-bromo-5-chloro-7-((thiophen-2-ylmethyl)amino)thieno[3,2- |
| | b]pyridin-2-yl)cyclohex-2-en-1-ol |
| 154 | 2-((1R,2R)-2-aminocyclopentyl)-5-chloro-3-iodo-N-(thiophen-2- |
| | ylmethyl)thieno[3,2-b]pyridin-7-amine |
| 155 | 2-((1R,2R)-2-aminocyclopentyl)-3-bromo-5-chloro-N-(furan-2- |
| | ylmethyl)thieno[3,2-b]pyridin-7-amine |
| 156 | 2-((2R,3R)-3-aminotetrahydro-2H-pyran-2-yl)-N-benzyl-3,5- |
| | dichlorothieno[3,2-b]pyridin-7-amine |
| 157 | 2-((1R,2R)-2-aminocycloheptyl)-5-chloro-3-iodo-N-(thiophen-2- |
| | ylmethyl)thieno[3,2-b]pyridin-7-amine |
| 158 | 2-((1R,2R)-2-aminocyclooctyl)-5-chloro-3-ethyl-N-(thiophen-2- |
| | ylmethyl)thieno[3,2-b]pyridin-7-amine |
| 159 | 2-((1S,2S)-2-aminocyclopentyl)-N-benzyl-3,5-dichlorothieno[3,2-b]pyridin-7- |
| | amine |
| 160 | 2-((1R,2R)-2-aminocyclopentyl)-N-benzyl-5-chloro-3-methylthieno[3,2- |
| | b]pyridin-7-amine |
| 161 | 2-((1R,2R)-2-aminocyclopentyl)-N-benzyl-3-bromo-5-chlorothieno[3,2- |
| | b]pyridin-7-amine |
| 162 | 2-((1S,2S)-2-aminocyclopentyl)-5-chloro-3-iodo-N-(thiophen-2- |
| | ylmethyl)thieno[3,2-b]pyridin-7-amine |
| 163 | 2-((1S,2S)-2-aminocycloheptyl)-5-chloro-3-iodo-N-(thiophen-2- |
| | ylmethyl)thieno[3,2-b]pyridin-7-amine |
| 164 | 2-((1S,2S)-2-aminocyclooctyl)-5-chloro-3-ethyl-N-(thiophen-2- |
| | ylmethyl)thieno[3,2-b]pyridin-7-amine |
| 165 | 2-((1S,2S)-2-aminocyclopentyl)-3-bromo-5-chloro-N-(furan-2- |
| | ylmethyl)thieno[3,2-b]pyridin-7-amine |
| 166 | 2-((1S,2S)-2-aminocyclopentyl)-N-benzyl-3-bromo-5-chlorothieno[3,2- |
| | b]pyridin-7-amine |
| 167 | 2-((1R,2R)-2-aminocyclopentyl)-N-benzyl-3,5-dichlorothieno[3,2-b]pyridin- |
| | 7-amine |
| 168 | 6-((1S,6S)-6-aminocyclohex-3-en-1-yl)-7-bromo-2-chloro-N-(furan-2- |
| | ylmethyl)thieno[3,2-d]pyrimidin-4-amine |
| 169 | 2-((1S,6S)-6-aminocyclohex-3-en-1-yl)-3,5-dichloro-N-(furan-2- |
| | ylmethyl)thieno[3,2-b]pyridin-7-amine |
| 170 | 6-((1S,6S)-6-aminocyclohex-3-en-1-yl)-2,7-dichloro-N-(furan-2- |
| | ylmethyl)thieno[3,2-d]pyrimidin-4-amine |
| 171 | 2-((1R,6R)-6-aminocyclohex-3-en-1-yl-2,2,3,4,5,5-d6)-5-chloro-N-(furan-2- |
| | ylmethyl)-3-methylthieno[3,2-b]pyridin-7-amine and 2-((1S,6S)-6- |
| | aminocyclohex-3-en-1-yl-2,2,3,4,5,5-d6)-5-chloro-N-(furan-2-ylmethyl)-3- |
| | methylthieno[3,2-b]pyridin-7-amine |
| 172 | 6-((1S,6S)-6-aminocyclohex-3-en-1-yl)-2-chloro-N-(furan-2-ylmethyl)-7- |
| | iodothieno[3,2-d]pyrimidin-4-amine |
| 173 | 2-((1S,2S)-2-aminocyclohexyl)-3,5-dichloro-N-(furan-2-ylmethyl)thieno[3,2- |
| | b]pyridin-7-amine |
| 174 | 2-((3R,4S)-4-aminotetrahydro-2H-pyran-3-yl)-3,5-dichloro-N-(furan-2- |
| | ylmethyl)thieno[3,2-b]pyridin-7-amine |
| 175 | 5-(2-((1R,6R)-6-aminocyclohex-3-en-1-yl)-5-chloro-7-((thiophen-2- |
| | ylmethyl)amino)thieno[3,2-b]pyridin-3-yl)pent-4-yn-1-ol and 5-(2-((1S,6S)-6- |
| | aminocyclohex-3-en-1-yl)-5-chloro-7-((thiophen-2- |
| | ylmethyl)amino)thieno[3,2-b]pyridin-3-yl)pent-4-yn-1-ol |
| 176 | 2-((1S,6S)-6-aminocyclohex-3-en-1-yl)-5-chloro-3-iodo-N-(thiophen-2- |
| | ylmethyl)thieno[3,2-b]pyridin-7-amine |
| 177 | 4-(2-((1R,6R)-6-aminocyclohex-3-en-1-yl)-5-chloro-7-((thiophen-2- |
| | ylmethyl)amino)thieno[3,2-b]pyridin-3-yl)but-3-yn-1-ol and 4-(2-((1S,6S)-6- |
| | aminocyclohex-3-en-1-yl)-5-chloro-7-((thiophen-2- |
| | ylmethyl)amino)thieno[3,2-b]pyridin-3-yl)but-3-yn-1-ol |
| 178 | 2-((1S,6S)-6-aminocyclohex-3-en-1-yl)-3,5-dichloro-N-(thiazol-2- |
| | ylmethyl)thieno[3,2-b]pyridin-7-amine |
| 179 | 2-((3R,4S)-4-aminotetrahydro-2H-pyran-3-yl)-3,5-dichloro-N-(thiophen-2- |
| | ylmethyl)thieno[3,2-b]pyridin-7-amine |
| 180 | 2-((1R,6R)-6-aminocyclohex-3-en-1-yl-2,2,3,4,5,5-d6)-3,5-dichloro-N-(furan- |
| | 2-ylmethyl)thieno[3,2-b]pyridin-7-amine and 2-((1S,6S)-6-aminocyclohex-3- |
| | en-1-yl-2,2,3,4,5,5-d6)-3,5-dichloro-N-(furan-2-ylmethyl)thieno[3,2- |
| | b]pyridin-7-amine |
| 181 | 2-((1R,2S,6R)-2-amino-6-fluorocyclohexyl)-3-bromo-5-chloro-N-(thiophen-2- |
| | ylmethyl)thieno[3,2-b]pyridin-7-amine and 2-((1S,2R,6S)-2-amino-6- |
| | fluorocyclohexyl)-3-bromo-5-chloro-N-(thiophen-2-ylmethyl)thieno[3,2- |
| | b]pyridin-7-amine |
| 182 | 6-((1S,6S)-6-aminocyclohex-3-en-1-yl)-2-chloro-7-iodo-N-(thiophen-2- |
| | ylmethyl)thieno[3,2-d]pyrimidin-4-amine |
| 183 | 6-((1S,6S)-6-aminocyclohex-3-en-1-yl-2,2,3,4,5,5-d6)-7-bromo-2-chloro-N- |
| | (thiophen-2-ylmethyl)thieno[3,2-d]pyrimidin-4-amine |
| 184 | 2-((1S,6S)-6-aminocyclohex-3-en-1-yl)-5-chloro-N-(furan-2-ylmethyl)-3- |
| | methylthieno[3,2-b]pyridin-7-amine |
| 185 | 2-((1R,6R)-6-aminocyclohex-3-en-1-yl)-3,5-dichloro-N-(thiophen-2- |
| | ylmethyl)thieno[3,2-b]pyridin-7-amine and 2-((1S,6S)-6-aminocyclohex-3-en- |
| | 1-yl)-3,5-dichloro-N-(thiophen-2-ylmethyl)thieno[3,2-b]pyridin-7-amine |
| 186 | 2-((1S,6S)-6-aminocyclohex-3-en-1-yl)-3-bromo-5-chloro-N-(thiophen-2- |
| | ylmethyl)thieno[3,2-b]pyridin-7-amine |
| 187 | 6-((1S,6S)-6-aminocyclohex-3-en-1-yl-2,2,3,4,5,5-d6)-2-chloro-N-(furan-2- |
| | ylmethyl)-7-methylthieno[3,2-d]pyrimidin-4-amine |
| 188 | 2-((1S,6S)-6-aminocyclohex-3-en-1-yl)-5-chloro-N-(furan-2-ylmethyl)-3- |
| | iodothieno[3,2-b]pyridin-7-amine |
| 189 | 2-((1R,2R)-2-aminocyclohexyl)-5-chloro-3-(prop-1-yn-1-yl)-N-(thiophen-2- |
| | ylmethyl)thieno[3,2-b]pyridin-7-amine and 2-((1S,2S)-2-aminocyclohexyl)-5- |
| | chloro-3-(prop-1-yn-1-yl)-N-(thiophen-2-ylmethyl)thieno[3,2-b]pyridin-7- |
| | amine |
| 190 | 2-((1S,6S)-6-aminocyclohex-3-en-1-yl)-N-benzyl-5-chloro-3-iodothieno[3,2- |
| | b]pyridin-7-amine |
| 191 | 6-((1S,6S)-6-aminocyclohex-3-en-1-yl)-7-bromo-2-chloro-N-(thiophen-2- |
| | ylmethyl)thieno[3,2-d]pyrimidin-4-amine |
| 192 | (2R,3S)-2-amino-3-(3-bromo-5-chloro-7-((thiophen-2- |
| | ylmethyl)amino)thieno[3,2-b]pyridin-2-yl)cyclohexan-1-one and (2S,3R)-2- |
| | amino-3-(3-bromo-5-chloro-7-((thiophen-2-ylmethyl)amino)thieno[3,2- |
| | b]pyridin-2-yl)cyclohexan-1-one |
| 193 | 6-((1S,6S)-6-aminocyclohex-3-en-1-yl)-2-chloro-N-(furan-2-ylmethyl)-7- |
| | methylthieno[3,2-d]pyrimidin-4-amine |
| 194 | 2-((1S,6S)-6-aminocyclohex-3-en-1-yl)-N-benzyl-3-bromo-5- |
| | chlorothieno[3,2-b]pyridin-7-amine |
| 195 | 2-((1R,6R)-6-aminocyclohex-3-en-1-yl-2,2,3,4,5,5-d6)-3,5-dichloro-N-(furan- |
| | 2-ylmethyl)thieno[3,2-b]pyridin-7-amine and 2-((1S,6S)-6-aminocyclohex-3- |
| | en-1-yl-2,2,3,4,5,5-d6)-3,5-dichloro-N-(furan-2-ylmethyl)thieno[3,2- |
| | b]pyridin-7-amine |
| 196 | 2-((1S,6S)-6-aminocyclohex-3-en-1-yl)-3,5-dichloro-N-(thiophen-2- |
| | ylmethyl)thieno[3,2-b]pyridin-7-amine |
| 197 | 2-((1S,2S)-2-aminocyclohexyl)-3-bromo-5-chloro-N-(pyridin-4- |
| | ylmethyl)thieno[3,2-b]pyridin-7-amine |
| 198 | 2-((1S,6S)-6-aminocyclohex-3-en-1-yl)-3-bromo-5-chloro-N-(pyridin-4- |
| | ylmethyl)thieno[3,2-b]pyridin-7-amine |
| 199 | 2-((1S,6S)-6-aminocyclohex-3-en-1-yl)-3,5-dichloro-N-(oxazol-2- |
| | ylmethyl)thieno[3,2-b]pyridin-7-amine |
| 200 | 6-((1R,6R)-6-aminocyclohex-3-en-1-yl-2,2,3,4,5,5-d6)-7-bromo-2-chloro-N- |
| | (furan-2-ylmethyl)thieno[3,2-d]pyrimidin-4-amine and 6-((1S,6S)-6- |
| | aminocyclohex-3-en-1-yl-2,2,3,4,5,5-d6)-7-bromo-2-chloro-N-(furan-2- |
| | ylmethyl)thieno[3,2-d]pyrimidin-4-amine |
| 201 | 6-((1R,6S)-6-amino-2,2-difluorocyclohexyl)-7-bromo-2-chloro-N-(furan-2- |
| | ylmethyl)thieno[3,2-d]pyrimidin-4-amine |
| 202 | 2-((1S,6S)-6-aminocyclohex-3-en-1-yl)-N-(but-2-yn-1-yl)-5-chloro-3- |
| | iodothieno[3,2-b]pyridin-7-amine |
| 203 | 2-((1S,6S)-6-aminocyclohex-3-en-1-yl)-3,5-dichloro-N-(pyridin-4- |
| | ylmethyl)thieno[3,2-b]pyridin-7-amine |
| 204 | 2-((3R,4S)-4-aminotetrahydro-2H-pyran-3-yl)-3-bromo-5-chloro-N-(thiophen- |
| | 2-ylmethyl)thieno[3,2-b]pyridin-7-amine and 2-((3S,4R)-4-aminotetrahydro- |
| | 2H-pyran-3-yl)-3-bromo-5-chloro-N-(thiophen-2-ylmethyl)thieno[3,2- |
| | b]pyridin-7-amine |
| 205 | 2-((1S,6S)-6-aminocyclohex-3-en-1-yl-2,2,3,4,5,5-d6)-5-chloro-3-methyl-N- |
| | (thiophen-2-ylmethyl)thieno[3,2-b]pyridin-7-amine |
| 206 | 2-((1S,6S)-6-aminocyclohex-3-en-1-yl)-3-bromo-5-chloro-N-(furan-2- |
| | ylmethyl)thieno[3,2-b]pyridin-7-amine |
| 207 | 2-((1S,6S)-6-aminocyclohex-3-en-1-yl)-N-benzyl-3,5-dichlorothieno[3,2- |
| | b]pyridin-7-amine |
| 208 | 2-((6R)-6-amino-2,2-difluorocyclohexyl)-5-chloro-3-(prop-1-yn-1-yl)-N- |
| | (thiophen-2-ylmethyl)thieno[3,2-b]pyridin-7-amine and 2-((6S)-6-amino-2,2- |
| | difluorocyclohexyl)-5-chloro-3-(prop-1-yn-1-yl)-N-(thiophen-2- |
| | ylmethyl)thieno[3,2-b]pyridin-7-amine |
| 209 | 2-((1R,6R)-6-aminocyclohex-3-en-1-yl)-5-chloro-3-iodo-N-(thiophen-2- |
| | ylmethyl)thieno[3,2-b]pyridin-7-amine and 2-((1S,6S)-6-aminocyclohex-3-en- |
| | 1-yl)-5-chloro-3-iodo-N-(thiophen-2-ylmethyl)thieno[3,2-b]pyridin-7-amine |
| 210 | 2-((1S,2S)-2-aminocyclohexyl)-N-benzyl-5-chloro-3-ethynylthieno[3,2- |
| | b]pyridin-7-amine |
| 211 | (R)-5-(2-((1S,6S)-6-aminocyclohex-3-en-1-yl)-5-chloro-7-((thiophen-2- |
| | ylmethyl)amino)thieno[3,2-b]pyridin-3-yl)pent-4-yne-1,2-diol |
| 212 | 2-((1S,6S)-6-aminocyclohex-3-en-1-yl-2,2,3,4,5,5-d6)-5-chloro-N-(furan-2- |
| | ylmethyl)-3-methylthieno[3,2-b]pyridin-7-amine |
| 213 | 4-(2-((2R,3R)-3-aminotetrahydro-2H-pyran-2-yl)-5-chloro-7-((thiophen-2- |
| | ylmethyl)amino)thieno[3,2-b]pyridin-3-yl)but-3-yn-1-ol and 4-(2-((2S,3S)-3- |
| | aminotetrahydro-2H-pyran-2-yl)-5-chloro-7-((thiophen-2- |
| | ylmethyl)amino)thieno[3,2-b]pyridin-3-yl)but-3-yn-1-ol |
| 214 | 6-((1S,6S)-6-aminocyclohex-3-en-1-yl)-2,7-dichloro-N-(pyridin-4- |
| | ylmethyl)thieno[3,2-d]pyrimidin-4-amine |
| 215 | 6-((2S,3S)-3-aminotetrahydro-2H-pyran-2-yl)-7-bromo-2-chloro-5- |
| | (difluoromethyl)-N-(furan-2-ylmethyl)-5H-pyrrolo[3,2-d]pyrimidin-4-amine |
| 216 | (S)-5-(2-((1S,6S)-6-aminocyclohex-3-en-1-yl)-5-chloro-7-((thiophen-2- |
| | ylmethyl)amino)thieno[3,2-b]pyridin-3-yl)pent-4-yne-1,2-diol |
| 217 | 2-((1S,6S)-6-aminocyclohex-3-en-1-yl)-3-bromo-5-chloro-N-(thiophen-3- |
| | ylmethyl)thieno[3,2-b]pyridin-7-amine |
| 218 | 2-((3R,4S)-4-aminotetrahydro-2H-pyran-3-yl)-N-benzyl-3,5- |
| | dichlorothieno[3,2-b]pyridin-7-amine |
| 219 | 6-((1S,6S)-6-aminocyclohex-3-en-1-yl)-N-(but-2-yn-1-yl)-2-chloro-7- |
| | iodothieno[3,2-d]pyrimidin-4-amine |
| 220 | 2-((1R,6R)-6-aminocyclohex-3-en-1-yl)-5-chloro-3-methyl-N-(thiophen-2- |
| | ylmethyl)thieno[3,2-b]pyridin-7-amine |
| 221 | (3S,4S)-4-amino-3-(3,5-dichloro-7-((furan-2-ylmethyl)amino)thieno[3,2- |
| | b]pyridin-2-yl)cyclohexan-1-one |
| 222 | 2-((1S,6S)-6-aminocyclohex-3-en-1-yl)-5-chloro-3-methyl-N-(thiophen-2- |
| | ylmethyl)thieno[3,2-b]pyridin-7-amine |
| 223 | 6-((1S,6S)-6-aminocyclohex-3-en-1-yl)-2-chloro-7-methyl-N-(thiazol-2- |
| | ylmethyl)thieno[3,2-d]pyrimidin-4-amine |
| 224 | 2-((2R,3R)-3-aminotetrahydro-2H-pyran-2-yl)-3-bromo-5-chloro-1- |
| | (difluoromethyl)-N-(thiophen-2-ylmethyl)-1H-pyrrolo[3,2-b]pyridin-7-amine |
| | and 2-((2S,3S)-3-aminotetrahydro-2H-pyran-2-yl)-3-bromo-5-chloro-1- |
| | (difluoromethyl)-N-(thiophen-2-ylmethyl)-1H-pyrrolo[3,2-b]pyridin-7-amine |
| 225 | 6-((2S,3S)-3-aminotetrahydro-2H-pyran-2-yl)-2-chloro-5-(difluoromethyl)-N- |
| | (furan-2-ylmethyl)-7-iodo-5H-pyrrolo[3,2-d]pyrimidin-4-amine |
| 226 | 2-((1R,2R)-2-aminocyclohexyl)-N-benzyl-5-chloro-3-(prop-1-yn-1- |
| | yl)thieno[3,2-b]pyridin-7-amine and 2-((1S,2S)-2-aminocyclohexyl)-N- |
| | benzyl-5-chloro-3-(prop-1-yn-1-yl)thieno[3,2-b]pyridin-7-amine |
| 227 | 2-((3R,4S)-3-aminotetrahydro-2H-pyran-4-yl)-5-chloro-3-(prop-1-yn-1-yl)-N- |
| | (thiophen-2-ylmethyl)thieno[3,2-b]pyridin-7-amine and 2-((3S,4R)-3- |
| | aminotetrahydro-2H-pyran-4-yl)-5-chloro-3-(prop-1-yn-1-yl)-N-(thiophen-2- |
| | ylmethyl)thieno[3,2-b]pyridin-7-amine |
| 228 | (1R,2R,3S)-2-amino-3-(3-bromo-5-chloro-7-((thiophen-2- |
| | ylmethyl)amino)thieno[3,2-b]pyridin-2-yl)cyclohexan-1-ol and (1S,2S,3R)-2- |
| | amino-3-(3-bromo-5-chloro-7-((thiophen-2-ylmethyl)amino)thieno[3,2- |
| | b]pyridin-2-yl)cyclohexan-1-ol |
| 229 | 6-((1S,6S)-6-aminocyclohex-3-en-1-yl)-N-benzyl-2-chloro-7-iodothieno[3,2- |
| | d]pyrimidin-4-amine |
| 230 | 2-((1S,6S)-6-aminocyclohex-3-en-1-yl)-3-bromo-N-(but-2-yn-1-yl)-5- |
| | chlorothieno[3,2-b]pyridin-7-amine |
| 231 | 2-((1S,2S)-2-aminocyclohexyl)-5-chloro-N-(furan-2-ylmethyl)-3- |
| | methylthieno[3,2-b]pyridin-7-amine |
| 232 | 2-((3R,4S)-4-aminotetrahydro-2H-pyran-3-yl)-5-chloro-3-methyl-N- |
| | (thiophen-2-ylmethyl)thieno[3,2-b]pyridin-7-amine |
| 233 | 2-((1S,6S)-6-amino-3,4-dimethylcyclohex-3-en-1-yl)-3,5-dichloro-N-(furan-2- |
| | ylmethyl)thieno[3,2-b]pyridin-7-amine |
| 234 | 6-((1S,6S)-6-aminocyclohex-3-en-1-yl)-2-chloro-7-methyl-N-(pyridin-4- |
| | ylmethyl)thieno[3,2-d]pyrimidin-4-amine |
| 235 | 6-(2-((1S,6S)-6-aminocyclohex-3-en-1-yl)-5-chloro-7-((thiophen-2- |
| | ylmethyl)amino)thieno[3,2-b]pyridin-3-yl)hex-5-yn-1-ol |
| 236 | 6-((1S,6S)-6-aminocyclohex-3-en-1-yl)-2,7-dichloro-N-((3-fluoropyridin-4- |
| | yl)methyl)thieno[3,2-d]pyrimidin-4-amine |
| 237 | 2-((1S,2S)-2-aminocyclohexyl)-N-benzyl-3,5-dichlorothieno[3,2-b]pyridin-7- |
| | amine |
| 238 | 2-((3R,4S)-3-aminotetrahydro-2H-pyran-4-yl)-3-bromo-5-chloro-N-(furan-2- |
| | ylmethyl)thieno[3,2-b]pyridin-7-amine |
| 239 | 2-((1S,6S)-6-aminocyclohex-3-en-1-yl)-3,5-dichloro-N-((3-fluoropyridin-4- |
| | yl)methyl)thieno[3,2-b]pyridin-7-amine |
| 240 | 6-((1R,6R)-6-aminocyclohex-3-en-1-yl)-2,7-dichloro-N-(thiophen-2- |
| | ylmethyl)thieno[3,2-d]pyrimidin-4-amine and 6-((1S,6S)-6-aminocyclohex-3- |
| | en-1-yl)-2,7-dichloro-N-(thiophen-2-ylmethyl)thieno[3,2-d]pyrimidin-4-amine |
| 241 | 6-((1S,6S)-6-aminocyclohex-3-en-1-yl)-N-benzyl-7-bromo-2- |
| | chlorothieno[3,2-d]pyrimidin-4-amine |
| 242 | 2-((1S,6S)-6-aminocyclohex-3-en-1-yl)-3-chloro-7-((furan-2- |
| | ylmethyl)amino)thieno[3,2-b]pyridine-5-carbonitrile |
| 243 | 6-((2R,3R)-3-aminotetrahydro-2H-pyran-2-yl)-2-chloro-N-(furan-2-ylmethyl)- |
| | 7-iodothieno[3,2-d]pyrimidin-4-amine and 6-((2S,3S)-3-aminotetrahydro-2H- |
| | pyran-2-yl)-2-chloro-N-(furan-2-ylmethyl)-7-iodothieno[3,2-d]pyrimidin-4- |
| | amine |
| 244 | 6-((2S,3S)-3-aminotetrahydro-2H-pyran-2-yl)-2,7-dichloro-5- |
| | (difluoromethyl)-N-(furan-2-ylmethyl)-5H-pyrrolo[3,2-d]pyrimidin-4-amine |
| 245 | 6-((3R,4S)-3-aminotetrahydro-2H-pyran-4-yl)-2-chloro-N-(furan-2-ylmethyl)- |
| | 7-iodothieno[3,2-d]pyrimidin-4-amine and 6-((3S,4R)-3-aminotetrahydro-2H- |
| | pyran-4-yl)-2-chloro-N-(furan-2-ylmethyl)-7-iodothieno[3,2-d]pyrimidin-4- |
| | amine |
| 246 | 2-((1S,6S)-6-aminocyclohex-3-en-1-yl)-3-methyl-7-((thiophen-2- |
| | ylmethyl)amino)thieno[3,2-b]pyridine-5-carbonitrile |
| 247 | 2-((1S,6S)-6-aminocyclohex-3-en-1-yl)-5-chloro-3-methyl-N-(thiophen-3- |
| | ylmethyl)thieno[3,2-b]pyridin-7-amine |
| 248 | 6-((2S,3S)-3-aminotetrahydro-2H-pyran-2-yl)-7-bromo-2-chloro-5- |
| | (difluoromethyl)-N-(thiophen-2-ylmethyl)-5H-pyrrolo[3,2-d]pyrimidin-4- |
| | amine |
| 249 | 2-((1S,6S)-6-amino-3,4-dimethylcyclohex-3-en-1-yl)-3-bromo-N-(but-2-yn-1- |
| | yl)-5-chlorothieno[3,2-b]pyridin-7-amine |
| 250 | 2-((1S,6S)-6-aminocyclohex-3-en-1-yl)-N-(but-2-yn-1-yl)-3,5- |
| | dichlorothieno[3,2-b]pyridin-7-amine |
| 251 | 2-((2S,3S)-3-aminotetrahydro-2H-pyran-2-yl)-3-bromo-5-chloro-N-(pyridin- |
| | 4-ylmethyl)thieno[3,2-b]pyridin-7-amine |
| 252 | 3-(2-((2R,3R)-3-aminotetrahydro-2H-pyran-2-yl)-5-chloro-7-((thiophen-2- |
| | ylmethyl)amino)thieno[3,2-b]pyridin-3-yl)prop-2-yn-1-ol and 3-(2-((2S,3S)-3- |
| | aminotetrahydro-2H-pyran-2-yl)-5-chloro-7-((thiophen-2- |
| | ylmethyl)amino)thieno[3,2-b]pyridin-3-yl)prop-2-yn-1-ol |
| 253 | 6-((1S,6S)-6-aminocyclohex-3-en-1-yl)-2-chloro-N-((3-fluoropyridin-4- |
| | yl)methyl)-7-methylthieno[3,2-d]pyrimidin-4-amine |
| 254 | 6-((2S,3S)-3-aminotetrahydro-2H-pyran-2-yl)-2-chloro-5-(difluoromethyl)-7- |
| | iodo-N-(thiophen-2-ylmethyl)-5H-pyrrolo[3,2-d]pyrimidin-4-amine |
| 255 | 3-(6-((2R,3R)-3-aminotetrahydro-2H-pyran-2-yl)-2-chloro-4-((furan-2- |
| | ylmethyl)amino)thieno[3,2-d]pyrimidin-7-yl)prop-2-yn-1-ol and 3-(6- |
| | ((2S,3S)-3-aminotetrahydro-2H-pyran-2-yl)-2-chloro-4-((furan-2- |
| | ylmethyl)amino)thieno[3,2-d]pyrimidin-7-yl)prop-2-yn-1-ol |
| 256 | 2-((1S,6S)-6-aminocyclohex-3-en-1-yl)-3-bromo-5-chloro-N-(thiophen-2- |
| | ylmethyl)thieno[3,2-b]pyridin-7-amine and 2-((1R,6R)-6-aminocyclohex-3- |
| | en-1-yl)-3-bromo-5-chloro-N-(thiophen-2-ylmethyl)thieno[3,2-b]pyridin-7- |
| | amine |
| 257 | 2-((1S,2S)-2-aminocyclohexyl)-5-chloro-3-((difluoromethoxy)methyl)-N- |
| | (thiophen-2-ylmethyl)thieno[3,2-b]pyridin-7-amine and 2-((1R,2R)-2- |
| | aminocyclohexyl)-5-chloro-3-((difluoromethoxy)methyl)-N-(thiophen-2- |
| | ylmethyl)thieno[3,2-b]pyridin-7-amine |
| 258 | 2-((1S,6S)-6-aminocyclohex-3-en-1-yl)-N-benzyl-5-chloro-3- |
| | methylthieno[3,2-b]pyridin-7-amine |
| 259 | 2-((2R,3R)-3-amino-5-methylenetetrahydro-2H-pyran-2-yl)-3-bromo-5- |
| | chloro-N-(thiophen-2-ylmethyl)thieno[3,2-b]pyridin-7-amine and 2-((2S,3S)- |
| | 3-amino-5-methylenetetrahydro-2H-pyran-2-yl)-3-bromo-5-chloro-N- |
| | (thiophen-2-ylmethyl)thieno[3,2-b]pyridin-7-amine |
| 260 | 2-((3R,4S)-4-aminotetrahydro-2H-pyran-3-yl)-5-chloro-N-(furan-2-ylmethyl)- |
| | 3-methylthieno[3,2-b]pyridin-7-amine |
| 261 | 2-((1S,6S)-6-aminocyclohex-3-en-1-yl-2,2,3,4,5,5-d6)-5-chloro-3-methyl-N- |
| | (thiophen-2-ylmethyl)thieno[3,2-b]pyridin-7-amine |
| 262 | 2-((2S,3S)-3-aminotetrahydro-2H-pyran-2-yl)-3,5-dichloro-1- |
| | (difluoromethyl)-N-(thiophen-2-ylmethyl)-1H-pyrrolo[3,2-b]pyridin-7-amine |
| 263 | 6-((1S,6S)-6-aminocyclohex-3-en-1-yl)-N-benzyl-2,7-dichlorothieno[3,2- |
| | d]pyrimidin-4-amine |
| 264 | 5-(5-chloro-2-((1S,2S)-2-(methylamino)cyclohexyl)-7-((thiophen-2- |
| | ylmethyl)amino)thieno[3,2-b]pyridin-3-yl)pent-4-yn-1-ol |
| 265 | 2-((3R,4S)-3-aminotetrahydro-2H-pyran-4-yl)-5-chloro-3-iodo-N-(thiazol-2- |
| | ylmethyl)thieno[3,2-b]pyridin-7-amine |
| 266 | 2-((2R,3R)-3-aminotetrahydro-2H-pyran-2-yl)-5-chloro-3-iodo-N-(thiophen- |
| | 2-ylmethyl)thieno[3,2-b]pyridin-7-amine and 2-((2S,3S)-3-aminotetrahydro- |
| | 2H-pyran-2-yl)-5-chloro-3-iodo-N-(thiophen-2-ylmethyl)thieno[3,2-b]pyridin- |
| | 7-amine |
| 267 | 2-((1S,6S)-6-aminocyclohex-3-en-1-yl)-5-chloro-N-(thiophen-2-ylmethyl)-3- |
| | vinylthieno[3,2-b]pyridin-7-amine |
| 268 | 6-((1S,6S)-6-aminocyclohex-3-en-1-yl)-2-chloro-7-methyl-N-(thiophen-2- |
| | ylmethyl)thieno[3,2-d]pyrimidin-4-amine |
| 269 | 2-((1R,6S)-6-amino-2,2-difluorocyclohexyl)-3-bromo-5-chloro-N-(thiophen- |
| | 2-ylmethyl)thieno[3,2-b]pyridin-7-amine and 2-((1S,6R)-6-amino-2,2- |
| | difluorocyclohexyl)-3-bromo-5-chloro-N-(thiophen-2-ylmethyl)thieno[3,2- |
| | b]pyridin-7-amine |
| 270 | 2-((2S,3S)-3-aminotetrahydro-2H-pyran-2-yl)-3-bromo-5-chloro-1- |
| | (difluoromethyl)-N-(furan-2-ylmethyl)-1H-pyrrolo[3,2-b]pyridin-7-amine |
| 271 | 6-((2S,3S)-3-aminotetrahydro-2H-pyran-2-yl)-2,7-dichloro-5- |
| | (difluoromethyl)-N-(furan-2-ylmethyl)-5H-pyrrolo[3,2-d]pyrimidin-4-amine |
| 272 | 2-((3R,4S)-3-aminotetrahydro-2H-pyran-4-yl)-3,5-dichloro-N-(thiophen-2- |
| | ylmethyl)thieno[3,2-b]pyridin-7-amine and 2-((3S,4R)-3-aminotetrahydro-2H- |
| | pyran-4-yl)-3,5-dichloro-N-(thiophen-2-ylmethyl)thieno[3,2-b]pyridin-7- |
| | amine |
| 273 | 2-((2S,3S)-3-aminotetrahydro-2H-pyran-2-yl)-3,5-dichloro-1- |
| | (difluoromethyl)-N-(furan-2-ylmethyl)-1H-pyrrolo[3,2-b]pyridin-7-amine |
| 274 | (4S,5S)-5-amino-4-(5-chloro-3-methyl-7-((thiophen-2- |
| | ylmethyl)amino)thieno[3,2-b]pyridin-2-yl)cyclohex-1-ene-1-carboxamide |
| 275 | 2-((3R,4S)-3-aminotetrahydro-2H-pyran-4-yl)-5-chloro-3-iodo-N-(thiophen-2- |
| | ylmethyl)thieno[3,2-b]pyridin-7-amine and 2-((3S,4R)-3-aminotetrahydro-2H- |
| | pyran-4-yl)-5-chloro-3-iodo-N-(thiophen-2-ylmethyl)thieno[3,2-b]pyridin-7- |
| | amine |
| 276 | 6-((1S,6S)-6-aminocyclohex-3-en-1-yl)-2-chloro-5-(difluoromethyl)-N-(furan- |
| | 2-ylmethyl)-7-iodo-5H-pyrrolo[3,2-d]pyrimidin-4-amine |
| 277 | ethyl (4S,5S)-5-amino-4-(5-chloro-3-methyl-7-((thiophen-2- |
| | ylmethyl)amino)thieno[3,2-b]pyridin-2-yl)cyclohex-1-ene-1-carboxylate |
| 278 | 2-((1R,6R)-6-aminocyclohex-3-en-1-yl-2,2,3,4,5,5-d6)-N-benzyl-5-chloro-3- |
| | methylthieno[3,2-b]pyridin-7-amine and 2-((1S,6S)-6-aminocyclohex-3-en-1- |
| | yl-2,2,3,4,5,5-d6)-N-benzyl-5-chloro-3-methylthieno[3,2-b]pyridin-7-amine |
| 279 | 2-((2R)-2-aminocyclohexyl)-5-chloro-3-(difluoromethyl)-N-(thiophen-2- |
| | ylmethyl)thieno[3,2-b]pyridin-7-amine and 2-((2S)-2-aminocyclohexyl)-5- |
| | chloro-3-(difluoromethyl)-N-(thiophen-2-ylmethyl)thieno[3,2-b]pyridin-7- |
| | amine |
| 280 | 2-((1S,2S)-2-aminocyclohexyl)-N-benzyl-5-chloro-3-methylthieno[3,2- |
| | b]pyridin-7-amine |
| 281 | 2-((2S,3S)-3-aminotetrahydro-2H-pyran-2-yl)-3-bromo-5-chloro-1- |
| | (difluoromethyl)-N-(thiophen-2-ylmethyl)-1H-pyrrolo[3,2-b]pyridin-7-amine |
| 282 | 6-((1S,6S)-6-aminocyclohex-3-en-1-yl)-4-((furan-2-ylmethyl)amino)-7- |
| | methylthieno[3,2-d]pyrimidine-2-carbonitrile |
| 283 | 2-((1S,2S)-2-aminocyclohexyl)-N-(but-2-yn-1-yl)-5-chloro-3-iodothieno[3,2- |
| | b]pyridin-7-amine |
| 284 | 2-((2R,3R,5R)-3-amino-5-methyltetrahydro-2H-pyran-2-yl)-3-bromo-5- |
| | chloro-N-(thiophen-2-ylmethyl)thieno[3,2-b]pyridin-7-amine and 2- |
| | ((2S,3S,5S)-3-amino-5-methyltetrahydro-2H-pyran-2-yl)-3-bromo-5-chloro- |
| | N-(thiophen-2-ylmethyl)thieno[3,2-b]pyridin-7-amine |
| 285 | 2-((3R,4S)-3-aminotetrahydro-2H-pyran-4-yl)-3-bromo-5-chloro-N-(thiazol- |
| | 2-ylmethyl)thieno[3,2-b]pyridin-7-amine |
| 286 | 6-((1S,2S)-2-aminocyclohexyl)-N-(but-2-yn-1-yl)-2-chloro-7-iodothieno[3,2- |
| | d]pyrimidin-4-amine |
| 287 | 2-((1S,6S)-6-aminocyclohex-3-en-1-yl)-3-bromo-5-chloro-N-(2- |
| | fluorobenzyl)thieno[3,2-b]pyridin-7-amine |
| 288 | 5-(5-chloro-2-((1R,6R)-6-(methylamino)cyclohex-3-en-1-yl)-7-((thiophen-2- |
| | ylmethyl)amino)thieno[3,2-b]pyridin-3-yl)pent-4-yn-1-ol and 5-(5-chloro-2- |
| | ((1S,6S)-6-(methylamino)cyclohex-3-en-1-yl)-7-((thiophen-2- |
| | ylmethyl)amino)thieno[3,2-b]pyridin-3-yl)pent-4-yn-1-ol |
| 289 | 2-((1S,6S)-6-aminocyclohex-3-en-1-yl)-7-((furan-2-ylmethyl)amino)-3- |
| | methylthieno[3,2-b]pyridine-5-carbonitrile |
| 290 | 2-((1R,3R,4R,6S)-4-aminobicyclo[4.1.0]heptan-3-yl)-3-bromo-5-chloro-N- |
| | (thiophen-2-ylmethyl)thieno[3,2-b]pyridin-7-amine and 2-((1S,3S,4S,6R)-4- |
| | aminobicyclo[4.1.0]heptan-3-yl)-3-bromo-5-chloro-N-(thiophen-2- |
| | ylmethyl)thieno[3,2-b]pyridin-7-amine |
| 291 | 2-((1R,6S)-6-amino-2,2-difluorocyclohexyl)-3,5-dichloro-N-(thiophen-2- |
| | ylmethyl)thieno[3,2-b]pyridin-7-amine |
| 292 | 2-((1S,6S)-6-aminocyclohex-3-en-1-yl-2,2,3,4,5,5-d6)-N-benzyl-5-chloro-3- |
| | methylthieno[3,2-b]pyridin-7-amine |
| 293 | 5-(2-chloro-6-((1R,6R)-6-(methylamino)cyclohex-3-en-1-yl)-4-((thiophen-2- |
| | ylmethyl)amino)thieno[3,2-d]pyrimidin-7-yl)pent-4-yn-1-ol and 5-(2-chloro- |
| | 6-((1S,6S)-6-(methylamino)cyclohex-3-en-1-yl)-4-((thiophen-2- |
| | ylmethyl)amino)thieno[3,2-d]pyrimidin-7-yl)pent-4-yn-1-ol |
| 294 | 2-((1R,5S,6R)-6-amino-5-methylcyclohex-3-en-1-yl)-3-bromo-5-chloro-N- |
| | (thiophen-2-ylmethyl)thieno[3,2-b]pyridin-7-amine and 2-((1S,5R,6S)-6- |
| | amino-5-methylcyclohex-3-en-1-yl)-3-bromo-5-chloro-N-(thiophen-2- |
| | ylmethyl)thieno[3,2-b]pyridin-7-amine |
| 295 | 2-((1R,2R)-2-aminocyclohexyl)-N-benzyl-5-chloro-3-iodothieno[3,2- |
| | b]pyridin-7-amine and 2-((1S,2S)-2-aminocyclohexyl)-N-benzyl-5-chloro-3- |
| | iodothieno[3,2-b]pyridin-7-amine |
| 296 | 2-((1R,2R)-2-aminocyclohexyl)-3,5-dichloro-N-(furan-2-ylmethyl)thieno[3,2- |
| | b]pyridin-7-amine |
| 297 | 6-((1R,6R)-6-aminocyclohex-3-en-1-yl-2,2,3,4,5,5-d6)-2-chloro-N-(furan-2- |
| | ylmethyl)-7-methylthieno[3,2-d]pyrimidin-4-amine and 6-((1S,6S)-6- |
| | aminocyclohex-3-en-1-yl-2,2,3,4,5,5-d6)-2-chloro-N-(furan-2-ylmethyl)-7- |
| | methylthieno[3,2-d]pyrimidin-4-amine |
| 298 | (1R,2R,3S)-2-amino-3-(3,5-dichloro-7-((thiophen-2- |
| | ylmethyl)amino)thieno[3,2-b]pyridin-2-yl)cyclohexan-1-ol |
| 299 | 2-((1R,6R)-6-aminocyclohex-3-en-1-yl)-3,5-dichloro-N-(furan-2- |
| | ylmethyl)thieno[3,2-b]pyridin-7-amine |
| 300 | 2-((1R,6R)-6-aminocyclohex-3-en-1-yl)-3-bromo-N-((E)-but-2-en-1-yl)-5- |
| | chlorothieno[3,2-b]pyridin-7-amine and 2-((1S,6S)-6-aminocyclohex-3-en-1- |
| | yl)-3-bromo-N-((E)-but-2-en-1-yl)-5-chlorothieno[3,2-b]pyridin-7-amine |
| 301 | 2-((1S,2S)-2-aminocyclohexyl)-3-bromo-N-(but-2-yn-1-yl)-5- |
| | chlorothieno[3,2-b]pyridin-7-amine |
| 302 | 2-((1S,6S)-6-aminocyclohex-3-en-1-yl)-N-((E)-but-2-en-1-yl)-3,5- |
| | dichlorothieno[3,2-b]pyridin-7-amine |
| 303 | 4-(5-chloro-2-((1S,6S)-6-(methylamino)cyclohex-3-en-1-yl)-7-((thiophen-2- |
| | ylmethyl)amino)thieno[3,2-b]pyridin-3-yl)but-3-yn-1-ol |
| 304 | 6-((1S,2S)-2-aminocyclohexyl)-2-chloro-5-(difluoromethyl)-N-(furan-2- |
| | ylmethyl)-7-iodo-5H-pyrrolo[3,2-d]pyrimidin-4-amine |
| 305 | 6-((2S,3S)-3-aminotetrahydro-2H-pyran-2-yl)-2,7-dichloro-5- |
| | (difluoromethyl)-N-(thiophen-2-ylmethyl)-5H-pyrrolo[3,2-d]pyrimidin-4- |
| | amine |
| 306 | 2-((1S,6S)-6-aminocyclohex-3-en-1-yl)-N-(but-2-yn-1-yl)-5-chloro-3- |
| | methylthieno[3,2-b]pyridin-7-amine |
| 307 | 2-((1S,6S)-6-aminocyclohex-3-en-1-yl)-5-chloro-3-(pyridin-3-ylethynyl)-N- |
| | (thiophen-2-ylmethyl)thieno[3,2-b]pyridin-7-amine |
| 308 | 2-((1S,2S)-2-aminocyclohexyl)-N-(but-2-yn-1-yl)-3,5-dichlorothieno[3,2- |
| | b]pyridin-7-amine |
| 309 | 5-(5-chloro-2-((2R,3R)-3-(methylamino)tetrahydro-2H-pyran-2-yl)-7- |
| | ((thiophen-2-ylmethyl)amino)thieno[3,2-b]pyridin-3-yl)pent-4-yn-1-ol and 5- |
| | (5-chloro-2-((2S,3S)-3-(methylamino)tetrahydro-2H-pyran-2-yl)-7-((thiophen- |
| | 2-ylmethyl)amino)thieno[3,2-b]pyridin-3-yl)pent-4-yn-1-ol |
| 310 | 2-((1R,6R)-6-aminocyclohex-3-en-1-yl)-3-bromo-5-chloro-N-(furan-2- |
| | ylmethyl)thieno[3,2-b]pyridin-7-amine |
| 311 | 2-((1S,2S)-2-aminocyclohexyl)-5-chloro-3-ethynyl-N-(2- |
| | fluorobenzyl)thieno[3,2-b]pyridin-7-amine |
| 312 | (4S,5S)-5-amino-4-(5-chloro-3-methyl-7-((thiophen-2- |
| | ylmethyl)amino)thieno[3,2-b]pyridin-2-yl)-N,N-dimethylcyclohex-1-ene-1- |
| | carboxamide |
| 313 | ((4S,5S)-5-amino-4-(5-chloro-3-methyl-7-((thiophen-2- |
| | ylmethyl)amino)thieno[3,2-b]pyridin-2-yl)cyclohex-1-en-1- |
| | yl)(morpholino)methanone |
| 314 | 2-((3R,4S)-3-aminotetrahydro-2H-pyran-4-yl)-3,5-dichloro-N-(furan-2- |
| | ylmethyl)thieno[3,2-b]pyridin-7-amine and 2-((3S,4R)-3-aminotetrahydro-2H- |
| | pyran-4-yl)-3,5-dichloro-N-(furan-2-ylmethyl)thieno[3,2-b]pyridin-7-amine |
| 315 | 2-((3R,4S)-3-aminotetrahydro-2H-pyran-4-yl)-3,5-dichloro-N-(thiophen-2- |
| | ylmethyl)thieno[3,2-b]pyridin-7-amine and 2-((3S,4R)-3-aminotetrahydro-2H- |
| | pyran-4-yl)-3,5-dichloro-N-(thiophen-2-ylmethyl)thieno[3,2-b]pyridin-7- |
| | amine |
| 316 | 2-((3R,4S)-3-aminotetrahydro-2H-pyran-4-yl)-3-bromo-5-chloro-N-(furan-2- |
| | ylmethyl)thieno[3,2-b]pyridin-7-amine and 2-((3S,4R)-3-aminotetrahydro-2H- |
| | pyran-4-yl)-3-bromo-5-chloro-N-(furan-2-ylmethyl)thieno[3,2-b]pyridin-7- |
| | amine |
| 317 | 6-((3R,4S)-3-aminotetrahydro-2H-pyran-4-yl)-2-chloro-N-(furan-2-ylmethyl)- |
| | 7-((trimethylsilyl)ethynyl)thieno[3,2-d]pyrimidin-4-amine and 6-((3S,4R)-3- |
| | aminotetrahydro-2H-pyran-4-yl)-2-chloro-N-(furan-2-ylmethyl)-7- |
| | ((trimethylsilyl)ethynyl)thieno[3,2-d]pyrimidin-4-amine |
| 318 | 6-((1R,2R)-2-aminocyclohexyl)-2-chloro-5-(difluoromethyl)-N-(furan-2- |
| | ylmethyl)-7-(prop-1-yn-1-yl)-5H-pyrrolo[3,2-d]pyrimidin-4-amine |
| 319 | 2-((3R,4S)-3-aminotetrahydro-2H-pyran-4-yl)-3,5-dichloro-N-(furan-2- |
| | ylmethyl)thieno[3,2-b]pyridin-7-amine and 2-((3S,4R)-3-aminotetrahydro-2H- |
| | pyran-4-yl)-3,5-dichloro-N-(furan-2-ylmethyl)thieno[3,2-b]pyridin-7-amine |
| 320 | 7-(2-((1S,6S)-6-aminocyclohex-3-en-1-yl)-5-chloro-7-((thiophen-2- |
| | ylmethyl)amino)thieno[3,2-b]pyridin-3-yl)hept-6-yn-1-ol |
| 321 | 2-((3R,4S)-4-aminotetrahydro-2H-pyran-3-yl)-N-benzyl-5-chloro-3- |
| | methylthieno[3,2-b]pyridin-7-amine |
| 322 | 2-((1S,6S)-6-aminocyclohex-3-en-1-yl-2,2,3,4,5,5-d6)-5-chloro-3-methyl-N- |
| | (phenylmethyl-d2)thieno[3,2-b]pyridin-7-amine |
| 323 | 6-((1R,6R)-6-aminocyclohex-3-en-1-yl)-2-chloro-N-(furan-2-ylmethyl)-7- |
| | iodothieno[3,2-d]pyrimidin-4-amine |
| 324 | 2-((1R,6R)-6-aminocyclohex-3-en-1-yl)-5-chloro-3-(propa-1,2-dien-1-yl)-N- |
| | (thiophen-2-ylmethyl)thieno[3,2-b]pyridin-7-amine and 2-((1S,6S)-6- |
| | aminocyclohex-3-en-1-yl)-5-chloro-3-(propa-1,2-dien-1-yl)-N-(thiophen-2- |
| | ylmethyl)thieno[3,2-b]pyridin-7-amine |
| 325 | 2-((1S,6S)-6-aminocyclohex-3-en-1-yl)-5-chloro-1-(difluoromethyl)-3-iodo- |
| | N-(thiophen-2-ylmethyl)-1H-pyrrolo[3,2-b]pyridin-7-amine |
| 326 | 6-((1S,6S)-6-aminocyclohex-3-en-1-yl)-N-benzyl-2-chloro-7- |
| | methylthieno[3,2-d]pyrimidin-4-amine |
| 327 | 2-((1R,6R)-6-aminocyclohex-3-en-1-yl)-5-chloro-N-(furan-2-ylmethyl)-3- |
| | iodothieno[3,2-b]pyridin-7-amine |
| 328 | 2-((3R,4S)-3-aminotetrahydro-2H-pyran-4-yl)-N-benzyl-3-bromo-5- |
| | chlorothieno[3,2-b]pyridin-7-amine and 2-((3S,4R)-3-aminotetrahydro-2H- |
| | pyran-4-yl)-N-benzyl-3-bromo-5-chlorothieno[3,2-b]pyridin-7-amine |
| 329 | (1R,5R,6S)-5-amino-6-(3-bromo-5-chloro-7-((thiophen-2- |
| | ylmethyl)amino)thieno[3,2-b]pyridin-2-yl)cyclohex-2-en-1-ol and (1S,5S,6R)- |
| | 5-amino-6-(3-bromo-5-chloro-7-((thiophen-2-ylmethyl)amino)thieno[3,2- |
| | b]pyridin-2-yl)cyclohex-2-en-1-ol |
| 330 | 6-((3R,4S)-3-aminotetrahydro-2H-pyran-4-yl)-2-chloro-7-ethynyl-N-(furan-2- |
| | ylmethyl)thieno[3,2-d]pyrimidin-4-amine and 6-((3S,4R)-3-aminotetrahydro- |
| | 2H-pyran-4-yl)-2-chloro-7-ethynyl-N-(furan-2-ylmethyl)thieno[3,2- |
| | d]pyrimidin-4-amine |
| 331 | 6-((1S,2S)-2-aminocyclohexyl)-2-chloro-5-(difluoromethyl)-N-(furan-2- |
| | ylmethyl)-7-(prop-1-yn-1-yl)-5H-pyrrolo[3,2-d]pyrimidin-4-amine |
| 332 | 2-((1R,6R)-6-aminocyclohex-3-en-1-yl)-3-bromo-5-chloro-N-(cyclopent-1- |
| | en-1-ylmethyl)thieno[3,2-b]pyridin-7-amine and 2-((1S,6S)-6-aminocyclohex- |
| | 3-en-1-yl)-3-bromo-5-chloro-N-(cyclopent-1-en-1-ylmethyl)thieno[3,2- |
| | b]pyridin-7-amine |
| 333 | 2-((1R,2R)-2-amino-5,5-difluorocyclohexyl)-5-chloro-3-methyl-N-(thiophen- |
| | 2-ylmethyl)thieno[3,2-b]pyridin-7-amine and 2-((1S,2S)-2-amino-5,5- |
| | difluorocyclohexyl)-5-chloro-3-methyl-N-(thiophen-2-ylmethyl)thieno[3,2- |
| | b]pyridin-7-amine |
| 334 | 6-((1S,2S)-2-aminocyclohexyl)-2,7-dichloro-5-(difluoromethyl)-N-(furan-2- |
| | ylmethyl)-5H-pyrrolo[3,2-d]pyrimidin-4-amine and 6-((1R,2R)-2- |
| | aminocyclohexyl)-2,7-dichloro-5-(difluoromethyl)-N-(furan-2-ylmethyl)-5H- |
| | pyrrolo[3,2-d]pyrimidin-4-amine |
| 335 | 2-((1R,2R,6S)-2-amino-6-fluorocyclohexyl)-3-bromo-5-chloro-N-(thiophen-2- |
| | ylmethyl)thieno[3,2-b]pyridin-7-amine and 2-((1S,2S,6R)-2-amino-6- |
| | fluorocyclohexyl)-3-bromo-5-chloro-N-(thiophen-2-ylmethyl)thieno[3,2- |
| | b]pyridin-7-amine |
| 336 | 5-(2-((1S,6S)-6-aminocyclohex-3-en-1-yl)-5-chloro-7-((furan-2- |
| | ylmethyl)(methyl)amino)thieno[3,2-b]pyridin-3-yl)pent-4-yn-1-ol |
| 337 | 2-((3R,4S)-3-aminotetrahydro-2H-pyran-4-yl)-3,5-dichloro-N-(thiazol-2- |
| | ylmethyl)thieno[3,2-b]pyridin-7-amine |
| 338 | 2-((1R,6S)-6-amino-2,2-difluorocyclohexyl)-5-chloro-3-methyl-N-(thiophen- |
| | 2-ylmethyl)thieno[3,2-b]pyridin-7-amine |
| 339 | 6-((3R,4S)-3-aminotetrahydro-2H-pyran-4-yl)-7-bromo-2-chloro-N-(furan-2- |
| | ylmethyl)thieno[3,2-d]pyrimidin-4-amine and 6-((3S,4R)-3-aminotetrahydro- |
| | 2H-pyran-4-yl)-7-bromo-2-chloro-N-(furan-2-ylmethyl)thieno[3,2- |
| | d]pyrimidin-4-amine |
| 340 | 6-((1S,6S)-6-aminocyclohex-3-en-1-yl)-2,7-dichloro-N-(thiazol-2- |
| | ylmethyl)thieno[3,2-d]pyrimidin-4-amine |
| 341 | 2-((1S,6S)-6-aminocyclohex-3-en-1-yl)-5-chloro-N-(2-fluorobenzyl)-3- |
| | methylthieno[3,2-b]pyridin-7-amine |
| 342 | 2-((1R,2S,3S)-2-amino-3-fluorocyclohexyl)-5-chloro-3-methyl-N-(thiophen- |
| | 2-ylmethyl)thieno[3,2-b]pyridin-7-amine and 2-((1S,2R,3R)-2-amino-3- |
| | fluorocyclohexyl)-5-chloro-3-methyl-N-(thiophen-2-ylmethyl)thieno[3,2- |
| | b]pyridin-7-amine |
| 343 | 2-((2S,3S)-3-aminotetrahydro-2H-pyran-2-yl)-3-bromo-N-(but-2-yn-1-yl)-5- |
| | chlorothieno[3,2-b]pyridin-7-amine |
| 344 | 2-((1R,2R)-2-aminocyclohexyl)-N-benzyl-5-chloro-3-ethynylthieno[3,2- |
| | b]pyridin-7-amine |
| 345 | 2-((1R,6R)-6-aminocyclohex-3-en-1-yl)-N-benzyl-5-chloro-3-iodothieno[3,2- |
| | b]pyridin-7-amine |
| 346 | 6-((2S,3S)-3-aminotetrahydro-2H-pyran-2-yl)-2,7-dichloro-5- |
| | (difluoromethyl)-N-(thiophen-2-ylmethyl)-5H-pyrrolo[3,2-d]pyrimidin-4- |
| | amine and 6-((2R,3R)-3-aminotetrahydro-2H-pyran-2-yl)-2,7-dichloro-5- |
| | (difluoromethyl)-N-(thiophen-2-ylmethyl)-5H-pyrrolo[3,2-d]pyrimidin-4- |
| | amine |
| 347 | 6-((3R,4S)-3-aminotetrahydro-2H-pyran-4-yl)-7-bromo-2-chloro-N-(thiophen- |
| | 2-ylmethyl)thieno[3,2-d]pyrimidin-4-amine and 6-((3S,4R)-3- |
| | aminotetrahydro-2H-pyran-4-yl)-7-bromo-2-chloro-N-(thiophen-2- |
| | ylmethyl)thieno[3,2-d]pyrimidin-4-amine |
| 348 | 6-((1S,6S)-6-aminocyclohex-3-en-1-yl)-N-benzyl-2-chloro-5- |
| | (difluoromethyl)-7-iodo-5H-pyrrolo[3,2-d]pyrimidin-4-amine |
| 349 | 5-(2-((1S,6S)-6-aminocyclohex-3-en-1-yl)-5-chloro-7-(methyl(thiophen-2- |
| | ylmethyl)amino)thieno[3,2-b]pyridin-3-yl)pent-4-yn-1-ol |
| 350 | 2-((3R,4S)-3-aminotetrahydro-2H-pyran-4-yl)-5-chloro-N-(thiophen-2- |
| | ylmethyl)-3-vinylthieno[3,2-b]pyridin-7-amine and 2-((3S,4R)-3- |
| | aminotetrahydro-2H-pyran-4-yl)-5-chloro-N-(thiophen-2-ylmethyl)-3- |
| | vinylthieno[3,2-b]pyridin-7-amine |
| 351 | 2-((3R,4S)-3-aminotetrahydro-2H-pyran-4-yl)-3-bromo-5-chloro-1-methyl-N- |
| | (thiophen-2-ylmethyl)-1H-pyrrolo[3,2-b]pyridin-7-amine |
| 352 | 2-((1S,6S)-6-aminocyclohex-3-en-1-yl)-N-benzyl-5-chloro-1- |
| | (difluoromethyl)-3-iodo-1H-pyrrolo[3,2-b]pyridin-7-amine |
| 353 | (4S,5S)-5-amino-4-(5-chloro-3-methyl-7-((thiophen-2- |
| | ylmethyl)amino)thieno[3,2-b]pyridin-2-yl)-N-phenylcyclohex-1-ene-1- |
| | carboxamide |
| 354 | 6-((3R,4S)-3-aminotetrahydro-2H-pyran-4-yl)-2,7-dichloro-N-(furan-2- |
| | ylmethyl)thieno[3,2-d]pyrimidin-4-amine and 6-((3S,4R)-3-aminotetrahydro- |
| | 2H-pyran-4-yl)-2,7-dichloro-N-(furan-2-ylmethyl)thieno[3,2-d]pyrimidin-4- |
| | amine |
| 355 | (1R,2S,3R)-3-amino-2-(3-bromo-5-chloro-7-((thiophen-2- |
| | ylmethyl)amino)thieno[3,2-b]pyridin-2-yl)cyclohexan-1-ol |
| 356 | 2-((1S,2S)-2-aminocyclohexyl)-5-chloro-N-(thiophen-2-ylmethyl)-3- |
| | (trifluoromethyl)thieno[3,2-b]pyridin-7-amine and 2-((1R,2R)-2- |
| | aminocyclohexyl)-5-chloro-N-(thiophen-2-ylmethyl)-3- |
| | (trifluoromethyl)thieno[3,2-b]pyridin-7-amine |
| 357 | 2-((1S,2S)-2-aminocyclohexyl)-5-chloro-3-ethynyl-N-(furan-2- |
| | ylmethyl)thieno[3,2-b]pyridin-7-amine |
| 358 | 6-((1R,6R)-6-aminocyclohex-3-en-1-yl)-2-chloro-5-(difluoromethyl)-N- |
| | (furan-2-ylmethyl)-7-methyl-5H-pyrrolo[3,2-d]pyrimidin-4-amine and 6- |
| | ((1S,6S)-6-aminocyclohex-3-en-1-yl)-2-chloro-5-(difluoromethyl)-N-(furan-2- |
| | ylmethyl)-7-methyl-5H-pyrrolo[3,2-d]pyrimidin-4-amine |
| 359 | 2-((1R,6R)-6-amino-2,2-difluorocyclohexyl)-3-bromo-5-chloro-N-(thiophen- |
| | 2-ylmethyl)thieno[3,2-b]pyridin-7-amine and 2-((1S,6S)-6-amino-2,2- |
| | difluorocyclohexyl)-3-bromo-5-chloro-N-(thiophen-2-ylmethyl)thieno[3,2- |
| | b]pyridin-7-amine |
| 360 | (2-((1S,2S)-2-aminocyclohexyl)-5-chloro-7-((thiophen-2- |
| | ylmethyl)amino)thieno[3,2-b]pyridin-3-yl)methanol and (2-((1R,2R)-2- |
| | aminocyclohexyl)-5-chloro-7-((thiophen-2-ylmethyl)amino)thieno[3,2- |
| | b]pyridin-3-yl)methanol |
| 361 | 2-((1S,2S)-2-aminocyclohexyl)-3-bromo-5-chloro-N-(cyclopent-1-en-1- |
| | ylmethyl)thieno[3,2-b]pyridin-7-amine |
| 362 | 6-((1R,6R)-6-aminocyclohex-3-en-1-yl)-2-chloro-7-iodo-N-(thiophen-2- |
| | ylmethyl)thieno[3,2-d]pyrimidin-4-amine |
| 363 | 2-((1S,2S)-2-aminocyclohexyl)-N-(but-2-yn-1-yl)-5-chloro-3- |
| | methylthieno[3,2-b]pyridin-7-amine |
| 364 | 2-((3R,4S)-3-aminotetrahydro-2H-pyran-4-yl)-3-bromo-5-chloro-N-(pyridin- |
| | 4-ylmethyl)thieno[3,2-b]pyridin-7-amine |
| 365 | 2-((1R,6R)-6-aminocyclohex-3-en-1-yl)-5-chloro-3-iodo-N-(thiophen-2- |
| | ylmethyl)thieno[3,2-b]pyridin-7-amine and 2-((1S,6S)-6-aminocyclohex-3-en- |
| | 1-yl)-5-chloro-3-iodo-N-(thiophen-2-ylmethyl)thieno[3,2-b]pyridin-7-amine |
| 366 | methyl (2-((1S,6S)-6-aminocyclohex-3-en-1-yl)-3-bromo-5-chlorothieno[3,2- |
| | b]pyridin-7-yl)glycinate |
| 367 | 2-((1R,6R)-6-aminocyclohex-3-en-1-yl)-5-chloro-3-methyl-N-(thiophen-2- |
| | ylmethyl)thieno[3,2-b]pyridin-7-amine and 2-((1S,6S)-6-aminocyclohex-3-en- |
| | 1-yl)-5-chloro-3-methyl-N-(thiophen-2-ylmethyl)thieno[3,2-b]pyridin-7- |
| | amine |
| 368 | 2-((1R,2R)-2-aminocyclohexyl)-N-benzyl-3,5-dichlorothieno[3,2-b]pyridin-7- |
| | amine |
| 369 | 2-((1R,6R)-6-aminocyclohex-3-en-1-yl-2,2,3,4,5,5-d6)-3,5-dichloro-N-(furan- |
| | 2-ylmethyl)thieno[3,2-b]pyridin-7-amine |
| 370 | 2-((1R,6S)-6-amino-2,2-difluorocyclohexyl)-3-bromo-N-(but-2-yn-1-yl)-5- |
| | chlorothieno[3,2-b]pyridin-7-amine and 2-((1S,6R)-6-amino-2,2- |
| | difluorocyclohexyl)-3-bromo-N-(but-2-yn-1-yl)-5-chlorothieno[3,2-b]pyridin- |
| | 7-amine |
| 371 | 2-((1R,5S,6S)-6-amino-5-methoxycyclohex-3-en-1-yl)-3-bromo-5-chloro-N- |
| | (thiophen-2-ylmethyl)thieno[3,2-b]pyridin-7-amine and 2-((1S,5R,6R)-6- |
| | amino-5-methoxycyclohex-3-en-1-yl)-3-bromo-5-chloro-N-(thiophen-2- |
| | ylmethyl)thieno[3,2-b]pyridin-7-amine |
| 372 | 6-((1R,6R)-6-aminocyclohex-3-en-1-yl-2,2,3,4,5,5-d6)-7-bromo-2-chloro-N- |
| | (thiophen-2-ylmethyl)thieno[3,2-d]pyrimidin-4-amine |
| 373 | 2-((1R,6R)-6-aminocyclohex-3-en-1-yl)-3-bromo-5-chloro-N-(pyridin-4- |
| | ylmethyl)thieno[3,2-b]pyridin-7-amine |
| 374 | (1R,2S,3R)-2-amino-3-(5-chloro-3-methyl-7-((thiophen-2- |
| | ylmethyl)amino)thieno[3,2-b]pyridin-2-yl)cyclohexan-1-ol and (1S,2R,3S)-2- |
| | amino-3-(5-chloro-3-methyl-7-((thiophen-2-ylmethyl)amino)thieno[3,2- |
| | b]pyridin-2-yl)cyclohexan-1-ol |
| 375 | 3-bromo-5-chloro-2-((1R,2R)-2-(methylamino)cyclohexyl)-N-(thiophen-2- |
| | ylmethyl)thieno[3,2-b]pyridin-7-amine and 3-bromo-5-chloro-2-((1S,2S)-2- |
| | (methylamino)cyclohexyl)-N-(thiophen-2-ylmethyl)thieno[3,2-b]pyridin-7- |
| | amine |
| 376 | 2-((1R,6R)-6-aminocyclohex-3-en-1-yl)-3,5-dichloro-N-((3-fluoropyridin-4- |
| | yl)methyl)thieno[3,2-b]pyridin-7-amine |
| 377 | (1R,5S,6R)-5-amino-6-(3,5-dichloro-7-((thiophen-2- |
| | ylmethyl)amino)thieno[3,2-b]pyridin-2-yl)cyclohex-2-en-1-ol |
| 378 | 2-((1S,6S)-6-aminocyclohex-3-en-1-yl)-N-((E)-but-2-en-1-yl)-5-chloro-3- |
| | methylthieno[3,2-b]pyridin-7-amine |
| 379 | 6-((1R,6R)-6-aminocyclohex-3-en-1-yl)-2-chloro-5-(difluoromethyl)-N- |
| | (furan-2-ylmethyl)-7-iodo-5H-pyrrolo[3,2-d]pyrimidin-4-amine |
| 380 | 2-((1R,6R)-6-aminocyclohex-3-en-1-yl)-3,5-dichloro-N-(thiazol-2- |
| | ylmethyl)thieno[3,2-b]pyridin-7-amine |
| 381 | 2-((3R,4S)-3-aminotetrahydro-2H-pyran-4-yl)-N-benzyl-3-bromo-5- |
| | chlorothieno[3,2-b]pyridin-7-amine and 2-((3S,4R)-3-aminotetrahydro-2H- |
| | pyran-4-yl)-N-benzyl-3-bromo-5-chlorothieno[3,2-b]pyridin-7-amine |
| 382 | 2-((1R,6R)-6-aminocyclohex-3-en-1-yl)-5-chloro-N-(furan-2-ylmethyl)-3- |
| | methylthieno[3,2-b]pyridin-7-amine |
| 383 | 2-((1R,6S)-6-amino-2,2-difluorocyclohexyl)-N-(but-2-yn-1-yl)-5-chloro-3- |
| | iodothieno[3,2-b]pyridin-7-amine and 2-((1S,6R)-6-amino-2,2- |
| | difluorocyclohexyl)-N-(but-2-yn-1-yl)-5-chloro-3-iodothieno[3,2-b]pyridin-7- |
| | amine |
| 384 | 2-((3S,4R)-3-aminotetrahydro-2H-pyran-4-yl)-3-bromo-5-chloro-1-methyl-N- |
| | (thiophen-2-ylmethyl)-1H-pyrrolo[3,2-b]pyridin-7-amine |
| 385 | 6-((1R,6R)-6-aminocyclohex-3-en-1-yl)-7-bromo-2-chloro-N-(thiophen-2- |
| | ylmethyl)thieno[3,2-d]pyrimidin-4-amine |
| 386 | 2-((1R,6R)-6-aminocyclohex-3-en-1-yl)-N-(but-2-yn-1-yl)-5-chloro-3- |
| | iodothieno[3,2-b]pyridin-7-amine |
| 387 | 5-chloro-3-iodo-2-((1R,6R)-6-(methylamino)cyclohex-3-en-1-yl)-N- |
| | (thiophen-2-ylmethyl)thieno[3,2-b]pyridin-7-amine and 5-chloro-3-iodo-2- |
| | ((1S,6S)-6-(methylamino)cyclohex-3-en-1-yl)-N-(thiophen-2- |
| | ylmethyl)thieno[3,2-b]pyridin-7-amine |
| 388 | 2-((3R,4S)-3-aminotetrahydro-2H-pyran-4-yl)-N-benzyl-3,5- |
| | dichlorothieno[3,2-b]pyridin-7-amine and 2-((3S,4R)-3-aminotetrahydro-2H- |
| | pyran-4-yl)-N-benzyl-3,5-dichlorothieno[3,2-b]pyridin-7-amine |
| 389 | 2-((3S,4R)-4-aminotetrahydro-2H-pyran-3-yl)-3,5-dichloro-N-(furan-2- |
| | ylmethyl)thieno[3,2-b]pyridin-7-amine |
| 390 | 6-((3R,4S)-3-aminotetrahydro-2H-pyran-4-yl)-2-chloro-N-(furan-2-ylmethyl)- |
| | 7-vinylthieno[3,2-d]pyrimidin-4-amine and 6-((3S,4R)-3-aminotetrahydro-2H- |
| | pyran-4-yl)-2-chloro-N-(furan-2-ylmethyl)-7-vinylthieno[3,2-d]pyrimidin-4- |
| | amine |
| 391 | 6-((1R,2R)-2-aminocyclohexyl)-2-chloro-5-(difluoromethyl)-N-(furan-2- |
| | ylmethyl)-7-iodo-5H-pyrrolo[3,2-d]pyrimidin-4-amine |
| 392 | 6-((2S,3S)-3-aminotetrahydro-2H-pyran-2-yl)-N-benzyl-2,7-dichloro-5- |
| | (difluoromethyl)-5H-pyrrolo[3,2-d]pyrimidin-4-amine and 6-((2R,3R)-3- |
| | aminotetrahydro-2H-pyran-2-yl)-N-benzyl-2,7-dichloro-5-(difluoromethyl)- |
| | 5H-pyrrolo[3,2-d]pyrimidin-4-amine |
| 393 | 2-((3R,4S)-3-aminotetrahydro-2H-pyran-4-yl)-5-chloro-7-((thiophen-2- |
| | ylmethyl)amino)thieno[3,2-b]pyridine-3-carbonitrile and 2-((3S,4R)-3- |
| | aminotetrahydro-2H-pyran-4-yl)-5-chloro-7-((thiophen-2- |
| | ylmethyl)amino)thieno[3,2-b]pyridine-3-carbonitrile |
| 394 | 2-((1S,6S)-6-aminocyclohex-3-en-1-yl)-5-chloro-3-((2-methoxypyridin-3- |
| | yl)ethynyl)-N-(thiophen-2-ylmethyl)thieno[3,2-b]pyridin-7-amine |
| 395 | 2-((1S,6S)-6-aminocyclohex-3-en-1-yl)-5-chloro-3-(pyridin-4-ylethynyl)-N- |
| | (thiophen-2-ylmethyl)thieno[3,2-b]pyridin-7-amine |
| 396 | 6-((1R,6R)-6-aminocyclohex-3-en-1-yl)-7-bromo-2-chloro-N-(furan-2- |
| | ylmethyl)thieno[3,2-d]pyrimidin-4-amine |
| 397 | 6-((1R,6R)-6-aminocyclohex-3-en-1-yl)-2,7-dichloro-N-(thiophen-2- |
| | ylmethyl)thieno[3,2-d]pyrimidin-4-amine and 6-((1S,6S)-6-aminocyclohex-3- |
| | en-1-yl)-2,7-dichloro-N-(thiophen-2-ylmethyl)thieno[3,2-d]pyrimidin-4-amine |
| 398 | 2-((3R,4S)-3-aminotetrahydro-2H-pyran-4-yl)-3-bromo-N-(but-2-yn-1-yl)-5- |
| | chlorothieno[3,2-b]pyridin-7-amine |
| 399 | 6-((1S,6S)-6-aminocyclohex-3-en-1-yl)-N-benzyl-2-chloro-5- |
| | (difluoromethyl)-7-methyl-5H-pyrrolo[3,2-d]pyrimidin-4-amine |
| 400 | 2-((1S,2S)-2-aminocyclohexyl)-5-chloro-3-(fluoromethyl)-N-(thiophen-2- |
| | ylmethyl)thieno[3,2-b]pyridin-7-amine and 2-((1R,2R)-2-aminocyclohexyl)-5- |
| | chloro-3-(fluoromethyl)-N-(thiophen-2-ylmethyl)thieno[3,2-b]pyridin-7- |
| | amine |
| 401 | 2-((1R,2R)-2-aminocyclohexyl)-5-chloro-N-(furan-2-ylmethyl)-3- |
| | methylthieno[3,2-b]pyridin-7-amine |
| 402 | 2-((1R,2S,6R)-6-amino-2-methylcyclohex-3-en-1-yl)-3-bromo-5-chloro-N- |
| | (thiophen-2-ylmethyl)thieno[3,2-b]pyridin-7-amine and 2-((1S,2R,6S)-6- |
| | amino-2-methylcyclohex-3-en-1-yl)-3-bromo-5-chloro-N-(thiophen-2- |
| | ylmethyl)thieno[3,2-b]pyridin-7-amine |
| 403 | 2-((1R,6R)-6-aminocyclohex-3-en-1-yl)-N-benzyl-3-bromo-5- |
| | chlorothieno[3,2-b]pyridin-7-amine |
| 404 | 2-((1R,6R)-6-aminocyclohex-3-en-1-yl)-N-benzyl-3,5-dichlorothieno[3,2- |
| | b]pyridin-7-amine |
| 405 | 2-((3S,4R)-4-aminotetrahydro-2H-pyran-3-yl)-3,5-dichloro-N-(thiophen-2- |
| | ylmethyl)thieno[3,2-b]pyridin-7-amine |
| 406 | 2-((3S,4R)-4-aminotetrahydro-2H-pyran-3-yl)-N-benzyl-3,5- |
| | dichlorothieno[3,2-b]pyridin-7-amine |
| 407 | 2-((3S,4R)-3-aminotetrahydro-2H-pyran-4-yl)-3,5-dichloro-N-(thiazol-2- |
| | ylmethyl)thieno[3,2-b]pyridin-7-amine |
| 408 | 2-((3S,4R)-3-aminotetrahydro-2H-pyran-4-yl)-5-chloro-3-iodo-N-(thiazol-2- |
| | ylmethyl)thieno[3,2-b]pyridin-7-amine |
| 409 | 2-((1S,6R)-6-amino-2,2-difluorocyclohexyl)-3,5-dichloro-N-(thiophen-2- |
| | ylmethyl)thieno[3,2-b]pyridin-7-amine |
| 410 | 2-((3S,4R)-3-aminotetrahydro-2H-pyran-4-yl)-3,5-dichloro-N-(furan-2- |
| | ylmethyl)thieno[3,2-b]pyridin-7-amine |
| 411 | 2-((1R,6R)-6-aminocyclohex-3-en-1-yl)-3-bromo-5-chloro-N-(2- |
| | fluorobenzyl)thieno[3,2-b]pyridin-7-amine |
| 412 | 6-((1S,6R)-6-amino-2,2-difluorocyclohexyl)-7-bromo-2-chloro-N-(furan-2- |
| | ylmethyl)thieno[3,2-d]pyrimidin-4-amine |
| 413 | 6-((1R,6R)-6-aminocyclohex-3-en-1-yl)-2-chloro-N-(furan-2-ylmethyl)-7- |
| | methylthieno[3,2-d]pyrimidin-4-amine |
| 414 | 6-((1R,6R)-6-aminocyclohex-3-en-1-yl)-N-benzyl-2-chloro-7-iodothieno[3,2- |
| | d]pyrimidin-4-amine |
| 415 | 2-((1R,6R)-6-aminocyclohex-3-en-1-yl)-3-bromo-5-chloro-N-(thiophen-3- |
| | ylmethyl)thieno[3,2-b]pyridin-7-amine |
| 416 | 6-((2R,3R)-3-aminotetrahydro-2H-pyran-2-yl)-2-chloro-5-(difluoromethyl)-7- |
| | iodo-N-(thiophen-2-ylmethyl)-5H-pyrrolo[3,2-d]pyrimidin-4-amine |
| 417 | 2-((1R,6R)-6-aminocyclohex-3-en-1-yl)-N-benzyl-3,5-dichloro-1- |
| | (difluoromethyl)-1H-pyrrolo[3,2-b]pyridin-7-amine |
| 418 | 6-((1S,2S)-2-aminocyclohexyl)-N-benzyl-2,7-dichloro-5-(difluoromethyl)-5H- |
| | pyrrolo[3,2-d]pyrimidin-4-amine and 6-((1R,2R)-2-aminocyclohexyl)-N- |
| | benzyl-2,7-dichloro-5-(difluoromethyl)-5H-pyrrolo[3,2-d]pyrimidin-4-amine |
| 419 | 2-((1R,6R)-6-aminocyclohex-3-en-1-yl)-5-chloro-3-methyl-N-(thiophen-3- |
| | ylmethyl)thieno[3,2-b]pyridin-7-amine |
| 420 | 2-((1S,6S)-6-aminocyclohex-3-en-1-yl)-N-benzyl-3,5-dichloro-1- |
| | (difluoromethyl)-1H-pyrrolo[3,2-b]pyridin-7-amine |
| 421 | 2-((1R,2R)-2-aminocyclohexyl)-3,5-dichloro-1-(difluoromethyl)-N-(thiophen- |
| | 2-ylmethyl)-1H-pyrrolo[3,2-b]pyridin-7-amine and 2-((1S,2S)-2- |
| | aminocyclohexyl)-3,5-dichloro-1-(difluoromethyl)-N-(thiophen-2-ylmethyl)- |
| | 1H-pyrrolo[3,2-b]pyridin-7-amine |
| 422 | 6-((2R,3R)-3-aminotetrahydro-2H-pyran-2-yl)-2-chloro-5-(difluoromethyl)-N- |
| | (furan-2-ylmethyl)-7-iodo-5H-pyrrolo[3,2-d]pyrimidin-4-amine |
| 423 | 6-((2R,3R)-3-aminotetrahydro-2H-pyran-2-yl)-2,7-dichloro-5- |
| | (difluoromethyl)-N-(thiophen-2-ylmethyl)-5H-pyrrolo[3,2-d]pyrimidin-4- |
| | amine |
| 424 | 6-((1R,6R)-6-aminocyclohex-3-en-1-yl)-N-benzyl-2-chloro-5- |
| | (difluoromethyl)-7-iodo-5H-pyrrolo[3,2-d]pyrimidin-4-amine |
| 425 | 2-((1R,6S)-6-amino-2,2-difluorocyclohexyl)-3-bromo-N-((E)-but-2-en-1-yl)- |
| | 5-chlorothieno[3,2-b]pyridin-7-amine and 2-((1S,6R)-6-amino-2,2- |
| | difluorocyclohexyl)-3-bromo-N-((E)-but-2-en-1-yl)-5-chlorothieno[3,2- |
| | b]pyridin-7-amine |
| 426 | 2-((2R,3R)-3-aminotetrahydro-2H-pyran-2-yl)-3-bromo-5-chloro-1- |
| | (difluoromethyl)-N-(furan-2-ylmethyl)-1H-pyrrolo[3,2-b]pyridin-7-amine |
| 427 | 2-((1R,6R)-6-aminocyclohex-3-en-1-yl)-5-chloro-1-(difluoromethyl)-3-iodo- |
| | N-(thiophen-2-ylmethyl)-1H-pyrrolo[3,2-b]pyridin-7-amine |
| 428 | 2-((1R,2R)-2-aminocyclohexyl)-3-bromo-N-(but-2-yn-1-yl)-5- |
| | chlorothieno[3,2-b]pyridin-7-amine |
| 429 | 2-((1R,6R)-6-aminocyclohex-3-en-1-yl)-3-bromo-N-(but-2-yn-1-yl)-5- |
| | chlorothieno[3,2-b]pyridin-7-amine |
| 430 | 2-((1R,2R)-2-aminocyclohexyl)-5-chloro-3-ethynyl-N-(2- |
| | fluorobenzyl)thieno[3,2-b]pyridin-7-amine |
| 431 | 2-((1R,6R)-6-aminocyclohex-3-en-1-yl)-3,5-dichloro-N-(thiophen-2- |
| | ylmethyl)thieno[3,2-b]pyridin-7-amine and 2-((1S,6S)-6-aminocyclohex-3-en- |
| | 1-yl)-3,5-dichloro-N-(thiophen-2-ylmethyl)thieno[3,2-b]pyridin-7-amine |
| 432 | 2-((1R,2R)-2-aminocyclohexyl)-3-bromo-5-chloro-N-(pyridin-4- |
| | ylmethyl)thieno[3,2-b]pyridin-7-amine |
| 433 | 2-((1R,6R)-6-aminocyclohex-3-en-1-yl)-3,5-dichloro-N-(pyridin-4- |
| | ylmethyl)thieno[3,2-b]pyridin-7-amine |
| 434 | 6-((1R,6R)-6-aminocyclohex-3-en-1-yl)-N-benzyl-7-bromo-2- |
| | chlorothieno[3,2-d]pyrimidin-4-amine |
| 435 | 6-((1R,6R)-6-aminocyclohex-3-en-1-yl)-2-chloro-N-((3-fluoropyridin-4- |
| | yl)methyl)-7-methylthieno[3,2-d]pyrimidin-4-amine |
| 436 | 6-((1R,6R)-6-aminocyclohex-3-en-1-yl)-2-chloro-7-methyl-N-(pyridin-4- |
| | ylmethyl)thieno[3,2-d]pyrimidin-4-amine |
| 437 | 6-((1R,6R)-6-aminocyclohex-3-en-1-yl)-2,7-dichloro-N-(furan-2- |
| | ylmethyl)thieno[3,2-d]pyrimidin-4-amine |
| 438 | 2-((1R,6R)-6-amino-3,4-dimethylcyclohex-3-en-1-yl)-3-bromo-N-(but-2-yn- |
| | 1-yl)-5-chlorothieno[3,2-b]pyridin-7-amine |
| 439 | 6-((1R,6R)-6-aminocyclohex-3-en-1-yl)-2,7-dichloro-N-((3-fluoropyridin-4- |
| | yl)methyl)thieno[3,2-d]pyrimidin-4-amine |
| 440 | 6-((1R,6R)-6-aminocyclohex-3-en-1-yl)-2,7-dichloro-N-(pyridin-4- |
| | ylmethyl)thieno[3,2-d]pyrimidin-4-amine |
| 441 | 6-((1R,6R)-6-aminocyclohex-3-en-1-yl)-2-chloro-7-methyl-N-(thiazol-2- |
| | ylmethyl)thieno[3,2-d]pyrimidin-4-amine |
| 442 | 2-((1S,2S)-2-aminocyclohexyl)-N-((Z)-but-2-en-1-yl)-3,5-dichlorothieno[3,2- |
| | b]pyridin-7-amine |
| 443 | 2-((1R,6R)-6-aminocyclohex-3-en-1-yl)-3-chloro-7-((furan-2- |
| | ylmethyl)amino)thieno[3,2-b]pyridine-5-carbonitrile |
| 444 | 2-((1R,6R)-6-amino-3,4-dimethylcyclohex-3-en-1-yl)-3,5-dichloro-N-(furan- |
| | 2-ylmethyl)thieno[3,2-b]pyridin-7-amine |
| 445 | ethyl (4R,5S)-5-amino-4-(5-chloro-3-methyl-7-((thiophen-2- |
| | ylmethyl)amino)thieno[3,2-b]pyridin-2-yl)cyclohex-1-ene-1-carboxylate |
| 446 | (4S,5S)-5-amino-4-(5-chloro-3-methyl-7-((thiophen-2- |
| | ylmethyl)amino)thieno[3,2-b]pyridin-2-yl)cyclohex-1-ene-1-carboxylic acid |
| 447 | 2-((1S,6S)-6-aminocyclohex-3-en-1-yl)-5-chloro-3-((3-chloropyridin-4- |
| | yl)ethynyl)-N-(thiophen-2-ylmethyl)thieno[3,2-b]pyridin-7-amine |
| 448 | 2-((1S,6S)-6-aminocyclohex-3-en-1-yl)-5-chloro-N-(thiophen-2- |
| | ylmethyl)thieno[3,2-b]pyridin-7-amine |
| 449 | 6-((1R,6R)-6-aminocyclohex-3-en-1-yl-2,2,3,4,5,5-d6)-2-chloro-N-(furan-2- |
| | ylmethyl)-7-methylthieno[3,2-d]pyrimidin-4-amine |
| 450 | 2-((1S,2R,5S,6S)-6-amino-2,5-dimethylcyclohex-3-en-1-yl)-3,5-dichloro-N- |
| | (furan-2-ylmethyl)thieno[3,2-b]pyridin-7-amine |
| 451 | 5-(2-((1R,6R)-6-aminocyclohex-3-en-1-yl)-5-chloro-7-(methyl(thiophen-2- |
| | ylmethyl)amino)thieno[3,2-b]pyridin-3-yl)pent-4-yn-1-ol |
| 452 | 5-(2-((1R,6R)-6-aminocyclohex-3-en-1-yl)-5-chloro-7-((furan-2- |
| | ylmethyl)(methyl)amino)thieno[3,2-b]pyridin-3-yl)pent-4-yn-1-ol |
| 453 | 5-(2-((1S,6S)-6-aminocyclohex-3-en-1-yl)-7-(benzyl(methyl)amino)-5- |
| | chlorothieno[3,2-b]pyridin-3-yl)pent-4-yn-1-ol |
| 454 | 5-(5-chloro-2-((1R,6R)-6-(dimethylamino)cyclohex-3-en-1-yl)-7-((thiophen- |
| | 2-ylmethyl)amino)thieno[3,2-b]pyridin-3-yl)pent-4-yn-1-ol and 5-(5-chloro-2- |
| | ((1S,6S)-6-(dimethylamino)cyclohex-3-en-1-yl)-7-((thiophen-2- |
| | ylmethyl)amino)thieno[3,2-b]pyridin-3-yl)pent-4-yn-1-ol, and |
| 455 | 2-((1R,6R)-6-aminocyclohex-3-en-1-yl)-3-bromo-5-chloro-N-((E)-pent-2-en- |
| | 1-yl)thieno[3,2-b]pyridin-7-amine and 2-((1S,6S)-6-aminocyclohex-3-en-1- |
| | yl)-3-bromo-5-chloro-N-((E)-pent-2-en-1-yl)thieno[3,2-b]pyridin-7-amine; |

wherein the form of the compound is a pharmaceutically acceptable salt, hydrate, solvate, racemate, enantiomer, diastereomer, stereoisomer, tautomer or isotope enriched form thereof.

One aspect includes a compound of Formula (I) or a form thereof selected from

| Compound | Compound Name |
|---|---|
| 24 | 2-((2R,3R)-3-aminotetrahydro-2H-pyran-2-yl)-3,5-dichloro-N-(2- |
| | fluorobenzyl)thieno[3,2-b]pyridin-7-amine hydrochloride and 2-((2S,3S)-3- |
| | aminotetrahydro-2H-pyran-2-yl)-3,5-dichloro-N-(2-fluorobenzyl)thieno[3,2- |
| | b]pyridin-7-amine hydrochloride |
| 26 | 2-((2R,3R)-3-aminotetrahydro-2H-pyran-2-yl)-N-benzyl-5-chlorothieno[3,2- |
| | b]pyridin-7-amine trifluoroacetate and 2-((2S,3S)-3-aminotetrahydro-2H- |
| | pyran-2-yl)-N-benzyl-5-chlorothieno[3,2-b]pyridin-7-amine trifluoroacetate |
| 27 | 2-(3-aminotetrahydro-2H-pyran-2-yl)-N-benzyl-3-bromo-5-chlorothieno[3,2- |
| | b]pyridin-7-amine trifluoroacetate |
| 28 | 2-((2R,3R)-3-aminotetrahydro-2H-pyran-2-yl)-5-chloro-N-(2- |
| | fluorobenzyl)thieno[3,2-b]pyridin-7-amine trifluoroacetate and 2-((2S,3S)-3- |
| | aminotetrahydro-2H-pyran-2-yl)-5-chloro-N-(2-fluorobenzyl)thieno[3,2- |
| | b]pyridin-7-amine trifluoroacetate |
| 29 | 2-((2R,3R)-3-aminotetrahydro-2H-pyran-2-yl)-3-bromo-5-chloro-N-(2- |
| | fluorobenzyl)thieno[3,2-b]pyridin-7-amine trifluoroacetate and 2-((2S,3S)-3- |
| | aminotetrahydro-2H-pyran-2-yl)-3-bromo-5-chloro-N-(2- |
| | fluorobenzyl)thieno[3,2-b]pyridin-7-amine trifluoroacetate |
| 30 | 2-((1R,2S)-2-amino-4,4-difluorocyclohexyl)-3-bromo-5-chloro-N-(thiophen- |
| | 2-ylmethyl)thieno[3,2-b]pyridin-7-amine hydrochloride and |
| 31 | (1R,2S)-2-amino-1-(3,5-dichloro-7-((thiophen-2-ylmethyl)amino)thieno[3,2- |
| | b]pyridin-2-yl)cyclohexan-1-ol hydrochloride 2-((1R,2S)-2-amino-4,4- |
| | difluorocyclohexyl)-3-bromo-5-chloro-N-(thiophen-2-ylmethyl)thieno[3,2- |
| | b]pyridin-7-amine hydrochloride |
| 32 | 2-((1R,2S)-2-amino-1-fluorocyclohexyl)-3,5-dichloro-N-(thiophen-2- |
| | ylmethyl)thieno[3,2-b]pyridin-7-amine hydrochloride |
| 33 | 2-((2S,3S)-3-aminotetrahydro-2H-pyran-2-yl)-5-chloro-N-(2-fluorobenzyl)-3- |
| | methylthieno[3,2-b]pyridin-7-amine trifluoroacetate |
| 37 | 2-((1R,2S)-2-amino-1-fluorocyclohexyl)-N-benzyl-3-bromo-5- |
| | chlorothieno[3,2-b]pyridin-7-amine hydrochloride |
| 38 | 2-((1R,2S)-2-amino-1-fluorocyclohexyl)-3,5-dichloro-N-(furan-2- |
| | ylmethyl)thieno[3,2-b]pyridin-7-amine hydrochloride |
| 41 | 2-((1R,2S)-2-amino-1-fluorocyclohexyl)-5-chloro-N-(furan-2-ylmethyl)-3- |
| | methylthieno[3,2-b]pyridin-7-amine hydrochloride |
| 46 | 2-((2R,3S)-3-aminopiperidin-2-yl)-3-bromo-5-chloro-N-(thiophen-2- |
| | ylmethyl)thieno[3,2-b]pyridin-7-amine formate and 2-((2S,3R)-3- |
| | aminopiperidin-2-yl)-3-bromo-5-chloro-N-(thiophen-2-ylmethyl)thieno[3,2- |
| | b]pyridin-7-amine formate |
| 47 | 2-((2R,3R)-3-aminopiperidin-2-yl)-3-bromo-5-chloro-N-(thiophen-2- |
| | ylmethyl)thieno[3,2-b]pyridin-7-amine formate and 2-((2S,3S)-3- |
| | aminopiperidin-2-yl)-3-bromo-5-chloro-N-(thiophen-2-ylmethyl)thieno[3,2- |
| | b]pyridin-7-amine formate |
| 58 | 2-((1R,2R)-2-aminocyclohexyl)-5-chloro-3-iodo-N-(thiophen-2- |
| | ylmethyl)thieno[3,2-b]pyridin-7-amine trifluoroacetate |
| 71 | 2-((1S,2S)-2-aminocyclohexyl)-5-chloro-3-methyl-N-(thiophen-2- |
| | ylmethyl)thieno[3,2-b]pyridin-7-amine formate |
| 85 | 2-((2R,3R)-3-aminobicyclo[3.2.1 ]octan-2-yl)-3-bromo-5-chloro-N-(thiophen- |
| | 2-ylmethyl)thieno[3,2-b]pyridin-7-amine formate and 2-((2S,3S)-3- |
| | aminobicyclo[3.2.1 ]octan-2-yl)-3-bromo-5-chloro-N-(thiophen-2- |
| | ylmethyl)thieno[3,2-b]pyridin-7-amine formate |
| 124 | 2-((2R,3R)-3-amino-1-phenylpiperidin-2-yl)-3-bromo-5-chloro-N-(thiophen- |
| | 2-ylmethyl)thieno[3,2-b]pyridin-7-amine formate and 2-((2S,3S)-3-amino-1- |
| | phenylpiperidin-2-yl)-3-bromo-5-chloro-N-(thiophen-2-ylmethyl)thieno[3,2- |
| | b]pyridin-7-amine formate |
| 125 | 2-((2R,3S)-3-amino-1-phenylpiperidin-2-yl)-3-bromo-5-chloro-N-(thiophen- |
| | 2-ylmethyl)thieno[3,2-b]pyridin-7-amine trifluoroacetate and 2-((2S,3R)-3- |
| | amino-1-phenylpiperidin-2-yl)-3-bromo-5-chloro-N-(thiophen-2- |
| | ylmethyl)thieno[3,2-b]pyridin-7-amine trifluoroacetate |
| 139 | 2-((3R,4S)-3-aminotetrahydro-2H-pyran-4-yl)-3-bromo-5-chloro-N-(thiophen- |
| | 2-ylmethyl)thieno[3,2-b]pyridin-7-amine trifluoroacetate |
| 140 | 2-((3S,4R)-3-aminotetrahydro-2H-pyran-4-yl)-3-bromo-5-chloro-N-(thiophen- |
| | 2-ylmethyl)thieno[3,2-b]pyridin-7-amine trifluoroacetate |
| 146 | 2-((3R,4R)-3-aminotetrahydro-2H-pyran-4-yl)-3-bromo-5-chloro-N- |
| | (thiophen-2-ylmethyl)thieno[3,2-b]pyridin-7-amine formate and 2-((3S,4S)-3- |
| | aminotetrahydro-2H-pyran-4-yl)-3-bromo-5-chloro-N-(thiophen-2- |
| | ylmethyl)thieno[3,2-b]pyridin-7-amine formate |
| 147 | 2-((4R,5R)-5-aminoazepan-4-yl)-3-bromo-5-chloro-N-(thiophen-2- |
| | ylmethyl)thieno[3,2-b]pyridin-7-amine trifluoroacetate and 2-((4S,5S)-5- |
| | aminoazepan-4-yl)-3-bromo-5-chloro-N-(thiophen-2-ylmethyl)thieno[3,2- |
| | b]pyridin-7-amine trifluoroacetate |
| 148 | 2-((4R,5S)-5-aminoazepan-4-yl)-3-bromo-5-chloro-N-(thiophen-2- |
| | ylmethyl)thieno[3,2-b]pyridin-7-amine trifluoroacetate and 2-((4S,5R)-5- |
| | aminoazepan-4-yl)-3-bromo-5-chloro-N-(thiophen-2-ylmethyl)thieno[3,2- |
| | b]pyridin-7-amine trifluoroacetate |
| 149 | 2-((1R,2R)-2-amino-5,5-dimethylcyclohexyl)-3-bromo-5-chloro-N-(thiophen- |
| | 2-ylmethyl)thieno[3,2-b]pyridin-7-amine formate and 2-((1S,2S)-2-amino-5,5- |
| | dimethylcyclohexyl)-3-bromo-5-chloro-N-(thiophen-2-ylmethyl)thieno[3,2- |
| | b]pyridin-7-amine formate |
| 150 | 2-((2R,3S)-3-aminotetrahydrofuran-2-yl)-3-bromo-5-chloro-N-(thiophen-2- |
| | ylmethyl)thieno[3,2-b]pyridin-7-amine formate and 2-((2S,3R)-3- |
| | aminotetrahydrofuran-2-yl)-3-bromo-5-chloro-N-(thiophen-2- |
| | ylmethyl)thieno[3,2-b]pyridin-7-amine formate |
| 151 | 2-((2R,3R)-3-aminotetrahydrofuran-2-yl)-3-bromo-5-chloro-N-(thiophen-2- |
| | ylmethyl)thieno[3,2-b]pyridin-7-amine formate and 2-((2,3S)-3- |
| | aminotetrahydrofuran-2-yl)-3-bromo-5-chloro-N-(thiophen-2- |
| | ylmethyl)thieno[3,2-b]pyridin-7-amine formate |
| 152 | 2-((1R,2R)-2-amino-4,4-dimethylcyclohexyl)-3-bromo-5-chloro-N-(thiophen- |
| | 2-ylmethyl)thieno[3,2-b]pyridin-7-amine formate and 2-((1S,2S)-2-amino-4,4- |
| | dimethylcyclohexyl)-3-bromo-5-chloro-N-(thiophen-2-ylmethyl)thieno[3,2- |
| | b]pyridin-7-amine formate |
| 153 | (1R,5S,6R)-6-amino-5-(3-bromo-5-chloro-7-((thiophen-2- |
| | ylmethyl)amino)thieno[3,2-b]pyridin-2-yl)cyclohex-2-en-1-ol formate and |
| | (1S,5R,6S)-6-amino-5-(3-bromo-5-chloro-7-((thiophen-2- |
| | ylmethyl)amino)thieno[3,2-b]pyridin-2-yl)cyclohex-2-en-1-ol formate |
| 175 | 5-(2-((1R,6R)-6-aminocyclohex-3-en-1-yl)-5-chloro-7-((thiophen-2- |
| | ylmethyl)amino)thieno[3,2-b]pyridin-3-yl)pent-4-yn-1-ol formate and 5-(2- |
| | ((1S,6S)-6-aminocyclohex-3-en-1-yl)-5-chloro-7-((thiophen-2- |
| | ylmethyl)amino)thieno[3,2-b]pyridin-3-yl)pent-4-yn-1-ol formate |
| 177 | 4-(2-((1R,6R)-6-aminocyclohex-3-en-1-yl)-5-chloro-7-((thiophen-2- |
| | ylmethyl)amino)thieno[3,2-b]pyridin-3-yl)but-3-yn-1-ol trifluoroacetate and |
| | 4-(2-((1S,6S)-6-aminocyclohex-3-en-1-yl)-5-chloro-7-((thiophen-2- |
| | ylmethyl)amino)thieno[3,2-b]pyridin-3-yl)but-3-yn-1-ol trifluoroacetate |
| 183 | 6-((1S,6S)-6-aminocyclohex-3-en-1-yl-2,2,3,4,5,5-d6)-7-bromo-2-chloro-N- |
| | (thiophen-2-ylmethyl)thieno[3,2-d]pyrimidin-4-amine trifluoroacetate |
| 185 | 2-((1R,6R)-6-aminocyclohex-3-en-1-yl)-3,5-dichloro-N-(thiophen-2- |
| | ylmethyl)thieno[3,2-b]pyridin-7-amine formate and 2-((1S,6S)-6- |
| | aminocyclohex-3-en-1-yl)-3,5-dichloro-N-(thiophen-2-ylmethyl)thieno[3,2- |
| | b]pyridin-7-amine formate |
| 187 | 6-((1S,6S)-6-aminocyclohex-3-en-1-yl-2,2,3,4,5,5-d6)-2-chloro-N-(furan-2- |
| | ylmethyl)-7-methylthieno[3,2-d]pyrimidin-4-amine trifluoroacetate |
| 189 | 2-((1R,2R)-2-aminocyclohexyl)-5-chloro-3-(prop-1-yn-1-yl)-N-(thiophen-2- |
| | ylmethyl)thieno[3,2-b]pyridin-7-amine formate and 2-((1S,2S)-2- |
| | aminocyclohexyl)-5-chloro-3-(prop-1-yn-1-yl)-N-(thiophen-2- |
| | ylmethyl)thieno[3,2-b]pyridin-7-amine formate |
| 192 | (2R,3S)-2-amino-3-(3-bromo-5-chloro-7-((thiophen-2- |
| | ylmethyl)amino)thieno[3,2-b]pyridin-2-yl)cyclohexan-1-one hydrochloride |
| | and (2S,3R)-2-amino-3-(3-bromo-5-chloro-7-((thiophen-2- |
| | ylmethyl)amino)thieno[3,2-b]pyridin-2-yl)cyclohexan-1-one hydrochloride |
| 195 | 2-((1R,6R)-6-aminocyclohex-3-en-1-yl-2,2,3,4,5,5-d6)-3,5-dichloro-N-(furan- |
| | 2-ylmethyl)thieno[3,2-b]pyridin-7-amine trifluoroacetate and 2-((1S,6S)-6- |
| | aminocyclohex-3-en-1-yl-2,2,3,4,5,5-d6)-3,5-dichloro-N-(furan-2- |
| | ylmethyl)thieno[3,2-b]pyridin-7-amine trifluoroacetate |
| 200 | 6-((1R,6R)-6-aminocyclohex-3-en-1-yl-2,2,3,4,5,5-d6)-7-bromo-2-chloro-N- |
| | (furan-2-ylmethyl)thieno[3,2-d]pyrimidin-4-amine formate and 6-((1S,6S)-6- |
| | aminocyclohex-3-en-1-yl-2,2,3,4,5,5-d6)-7-bromo-2-chloro-N-(furan-2- |
| | ylmethyl)thieno[3,2-d]pyrimidin-4-amine formate |
| 204 | 2-((3R,4S)-4-aminotetrahydro-2H-pyran-3-yl)-3-bromo-5-chloro-N-(thiophen- |
| | 2-ylmethyl)thieno[3,2-b]pyridin-7-amine trifluoroacetate and 2-((3S,4R)-4- |
| | aminotetrahydro-2H-pyran-3-yl)-3-bromo-5-chloro-N-(thiophen-2- |
| | ylmethyl)thieno[3,2-b]pyridin-7-amine trifluoroacetate |
| 205 | 2-((1S,6S)-6-aminocyclohex-3-en-1-yl-2,2,3,4,5,5-d6)-5-chloro-3-methyl-N- |
| | (thiophen-2-ylmethyl)thieno[3,2-b]pyridin-7-amine formate |
| 208 | 2-((6R)-6-amino-2,2-difluorocyclohexyl)-5-chloro-3-(prop-1-yn-1-yl)-N- |
| | (thiophen-2-ylmethyl)thieno[3,2-b]pyridin-7-amine trifluoroacetate and 2- |
| | ((6S)-6-amino-2,2-difluorocyclohexyl)-5-chloro-3-(prop-1-yn-1-yl)-N- |
| | (thiophen-2-ylmethyl)thieno[3,2-b]pyridin-7-amine trifluoroacetate |
| 209 | 2-((1R,6R)-6-aminocyclohex-3-en-1-yl)-5-chloro-3-iodo-N-(thiophen-2- |
| | ylmethyl)thieno[3,2-b]pyridin-7-amine formate and 2-((1S,6S)-6- |
| | aminocyclohex-3-en-1-yl)-5-chloro-3-iodo-N-(thiophen-2- |
| | ylmethyl)thieno[3,2-b]pyridin-7-amine formate |
| 211 | (R)-5-(2-((1S,6S)-6-aminocyclohex-3-en-1-yl)-5-chloro-7-((thiophen-2- |
| | ylmethyl)amino)thieno[3,2-b]pyridin-3-yl)pent-4-yne-1,2-diol trifluoroacetate |
| 212 | 2-((1S,6S)-6-aminocyclohex-3-en-1-yl-2,2,3,4,5,5-d6)-5-chloro-N-(furan-2- |
| | ylmethyl)-3-methylthieno[3,2-b]pyridin-7-amine trifluoroacetate |
| 213 | 4-(2-((2R,3R)-3-aminotetrahydro-2H-pyran-2-yl)-5-chloro-7-((thiophen-2- |
| | ylmethyl)amino)thieno[3,2-b]pyridin-3-yl)but-3-yn-1-ol trifluoroacetate and |
| | 4-(2-((2S,3S)-3-aminotetrahydro-2H-pyran-2-yl)-5-chloro-7-((thiophen-2- |
| | ylmethyl)amino)thieno[3,2-b]pyridin-3-yl)but-3-yn-1-ol trifluoroacetate |
| 216 | (S)-5-(2-((1S,6S)-6-aminocyclohex-3-en-1-yl)-5-chloro-7-((thiophen-2- |
| | ylmethyl)amino)thieno[3,2-b]pyridin-3-yl)pent-4-yne-1,2-diol trifluoroacetate |
| 226 | 2-((1R,2R)-2-aminocyclohexyl)-N-benzyl-5-chloro-3-(prop-1-yn-1- |
| | yl)thieno[3,2-b]pyridin-7-amine formate and 2-((1S,2S)-2-aminocyclohexyl)- |
| | N-benzyl-5-chloro-3-(prop-1-yn-1-yl)thieno[3,2-b]pyridin-7-amine formate |
| 227 | 2-((3R,4S)-3-aminotetrahydro-2H-pyran-4-yl)-5-chloro-3-(prop-1-yn-1-yl)-N- |
| | (thiophen-2-ylmethyl)thieno[3,2-b]pyridin-7-amine formate and 2-((3S,4R)-3- |
| | aminotetrahydro-2H-pyran-4-yl)-5-chloro-3-(prop-1-yn-1-yl)-N-(thiophen-2- |
| | ylmethyl)thieno[3,2-b]pyridin-7-amine formate |
| 228 | (1R,2R,3S)-2-amino-3-(3-bromo-5-chloro-7-((thiophen-2- |
| | ylmethyl)amino)thieno[3,2-b]pyridin-2-yl)cyclohexan-1-ol trifluoroacetate |
| | and (1S,2S,3R)-2-amino-3-(3-bromo-5-chloro-7-((thiophen-2- |
| | ylmethyl)amino)thieno[3,2-b]pyridin-2-yl)cyclohexan-1-ol trifluoroacetate |
| 235 | 6-(2-((1S,6S)-6-aminocyclohex-3-en-1-yl)-5-chloro-7-((thiophen-2- |
| | ylmethyl)amino)thieno[3,2-b]pyridin-3-yl)hex-5-yn-1-ol trifluoroacetate |
| 238 | 2-((3R,4S)-3-aminotetrahydro-2H-pyran-4-yl)-3-bromo-5-chloro-N-(furan-2- |
| | ylmethyl)thieno[3,2-b]pyridin-7-amine trifluoroacetate |
| 243 | 6-((2R,3R)-3-aminotetrahydro-2H-pyran-2-yl)-2-chloro-N-(furan-2-ylmethyl)- |
| | 7-iodothieno[3,2-d]pyrimidin-4-amine trifluoroacetate and 6-((2S,3S)-3- |
| | aminotetrahydro-2H-pyran-2-yl)-2-chloro-N-(furan-2-ylmethyl)-7- |
| | iodothieno[3,2-d]pyrimidin-4-amine trifluoroacetate |
| 245 | 6-((3R,4S)-3-aminotetrahydro-2H-pyran-4-yl)-2-chloro-N-(furan-2-ylmethyl)- |
| | 7-iodothieno[3,2-d]pyrimidin-4-amine formate and 6-((3S,4R)-3- |
| | aminotetrahydro-2H-pyran-4-yl)-2-chloro-N-(furan-2-ylmethyl)-7- |
| | iodothieno[3,2-d]pyrimidin-4-amine formate |
| 252 | 3-(2-((2R,3R)-3-aminotetrahydro-2H-pyran-2-yl)-5-chloro-7-((thiophen-2- |
| | ylmethyl)amino)thieno[3,2-b]pyridin-3-yl)prop-2-yn-1-ol trifluoroacetate and |
| | 3-(2-((2S,3S)-3-aminotetrahydro-2H-pyran-2-yl)-5-chloro-7-((thiophen-2- |
| | ylmethyl)amino)thieno[3,2-b]pyridin-3-yl)prop-2-yn-1-ol trifluoroacetate |
| 255 | 3-(6-((2R,3R)-3-aminotetrahydro-2H-pyran-2-yl)-2-chloro-4-((furan-2- |
| | ylmethyl)amino)thieno[3,2-d]pyrimidin-7-yl)prop-2-yn-1-ol trifluoroacetate |
| | and 3-(6-((2S,3S)-3-aminotetrahydro-2H-pyran-2-yl)-2-chloro-4-((furan-2- |
| | ylmethyl)amino)thieno[3,2-d]pyrimidin-7-yl)prop-2-yn-1-ol trifluoroacetate |
| 259 | 2-((2R,3R)-3-amino-5-methylenetetrahydro-2H-pyran-2-yl)-3-bromo-5- |
| | chloro-N-(thiophen-2-ylmethyl)thieno[3,2-b]pyridin-7-amine trifluoroacetate |
| | and 2-((2S,3S)-3-amino-5-methylenetetrahydro-2H-pyran-2-yl)-3-bromo-5- |
| | chloro-N-(thiophen-2-ylmethyl)thieno[3,2-b]pyridin-7-amine trifluoroacetate |
| 261 | 2-((1S,6S)-6-aminocyclohex-3-en-1-yl-2,2,3,4,5,5-d6)-5-chloro-3-methyl-N- |
| | (thiophen-2-ylmethyl)thieno[3,2-b]pyridin-7-amine trifluoroacetate |
| 264 | 5-(5-chloro-2-((1S,2S)-2-(methylamino)cyclohexyl)-7-((thiophen-2- |
| | ylmethyl)amino)thieno[3,2-b]pyridin-3-yl)pent-4-yn-1-ol trifluoroacetate |
| 266 | 2-((2R,3R)-3-aminotetrahydro-2H-pyran-2-yl)-5-chloro-3-iodo-N-(thiophen- |
| | 2-ylmethyl)thieno[3,2-b]pyridin-7-amine hydrochloride and 2-((2S,3S)-3- |
| | aminotetrahydro-2H-pyran-2-yl)-5-chloro-3-iodo-N-(thiophen-2- |
| | ylmethyl)thieno[3,2-b]pyridin-7-amine hydrochloride |
| 267 | 2-((1S,6S)-6-aminocyclohex-3-en-1-yl)-5-chloro-N-(thiophen-2-ylmethyl)-3- |
| | vinylthieno[3,2-b]pyridin-7-amine trifluoroacetate |
| 269 | 2-((1R,6S)-6-amino-2,2-difluorocyclohexyl)-3-bromo-5-chloro-N-(thiophen- |
| | 2-ylmethyl)thieno[3,2-b]pyridin-7-amine trifluoroacetate and 2-((1S,6R)-6- |
| | amino-2,2-difluorocyclohexyl)-3-bromo-5-chloro-N-(thiophen-2- |
| | ylmethyl)thieno[3,2-b]pyridin-7-amine trifluoroacetate |
| 272 | 2-((3R,4S)-3-aminotetrahydro-2H-pyran-4-yl)-3,5-dichloro-N-(thiophen-2- |
| | ylmethyl)thieno[3,2-b]pyridin-7-amine trifluoroacetate and 2-((3S,4R)-3- |
| | aminotetrahydro-2H-pyran-4-yl)-3,5-dichloro-N-(thiophen-2- |
| | ylmethyl)thieno[3,2-b]pyridin-7-amine trifluoroacetate |
| 275 | 2-((3R,4S)-3-aminotetrahydro-2H-pyran-4-yl)-5-chloro-3-iodo-N-(thiophen-2- |
| | ylmethyl)thieno[3,2-b]pyridin-7-amine formate and 2-((3S,4R)-3- |
| | aminotetrahydro-2H-pyran-4-yl)-5-chloro-3-iodo-N-(thiophen-2- |
| | ylmethyl)thieno[3,2-b]pyridin-7-amine formate |
| 284 | 2-((2R,3R,5R)-3-amino-5-methyltetrahydro-2H-pyran-2-yl)-3-bromo-5- |
| | chloro-N-(thiophen-2-ylmethyl)thieno[3,2-b]pyridin-7-amine trifluoroacetate |
| | and 2-((2S,3S,5S)-3-amino-5-methyltetrahydro-2H-pyran-2-yl)-3-bromo-5- |
| | chloro-N-(thiophen-2-ylmethyl)thieno[3,2-b]pyridin-7-amine trifluoroacetate |
| 288 | 5-(5-chloro-2-((1R,6R)-6-(methylamino)cyclohex-3-en-1-yl)-7-((thiophen-2- |
| | ylmethyl)amino)thieno[3,2-b]pyridin-3-yl)pent-4-yn-1-ol trifluoroacetate and |
| | 5-(5-chloro-2-((1S,6S)-6-(methylamino)cyclohex-3-en-1-yl)-7-((thiophen-2- |
| | ylmethyl)amino)thieno[3,2-b]pyridin-3-yl)pent-4-yn-1-ol trifluoroacetate; |
| 290 | 2-((1R,3R,4R,6S)-4-aminobicyclo[4.1.0]heptan-3-yl)-3-bromo-5-chloro-N- |
| | (thiophen-2-ylmethyl)thieno[3,2-b]pyridin-7-amine trifluoroacetate and 2- |
| | ((1S,3S,4S,6R)-4-aminobicyclo[4.1.0]heptan-3-yl)-3-bromo-5-chloro-N- |
| | (thiophen-2-ylmethyl)thieno[3,2-b]pyridin-7-amine trifluoroacetate |
| 292 | 2-((1S,6S)-6-aminocyclohex-3-en-1-yl-2,2,3,4,5,5-d6)-N-benzyl-5-chloro-3- |
| | methylthieno[3,2-b]pyridin-7-amine trifluoroacetate |
| 293 | 5-(2-chloro-6-((1R,6R)-6-(methylamino)cyclohex-3-en-1-yl)-4-((thiophen-2- |
| | ylmethyl)amino)thieno[3,2-d]pyrimidin-7-yl)pent-4-yn-1-ol trifluoroacetate |
| | and 5-(2-chloro-6-((1S,6S)-6-(methylamino)cyclohex-3-en-1-yl)-4-((thiophen- |
| | 2-ylmethyl)amino)thieno[3,2-d]pyrimidin-7-yl)pent-4-yn-1-ol trifluoroacetate |
| 295 | 2-((1R,2R)-2-aminocyclohexyl)-N-benzyl-5-chloro-3-iodothieno[3,2- |
| | b]pyridin-7-amine formate and 2-((1S,2S)-2-aminocyclohexyl)-N-benzyl-5- |
| | chloro-3-iodothieno[3,2-b]pyridin-7-amine formate |
| 300 | 2-((1R,6R)-6-aminocyclohex-3-en-1-yl)-3-bromo-N-((E)-but-2-en-1-yl)-5- |
| | chlorothieno[3,2-b]pyridin-7-amine formate and 2-((1S,6S)-6-aminocyclohex- |
| | 3-en-1-yl)-3-bromo-N-((E)-but-2-en-1-yl)-5-chlorothieno[3,2-b]pyridin-7- |
| | amine formate |
| 302 | 2-((1S,6S)-6-aminocyclohex-3-en-1-yl)-N-((E)-but-2-en-1-yl)-3,5- |
| | dichlorothieno[3,2-b]pyridin-7-amine formate |
| 303 | 4-(5-chloro-2-((1S,6S)-6-(methylamino)cyclohex-3-en-1-yl)-7-((thiophen-2- |
| | ylmethyl)amino)thieno[3,2-b]pyridin-3-yl)but-3-yn-1-ol trifluoroacetate |
| 307 | 2-((1S,6S)-6-aminocyclohex-3-en-1-yl)-5-chloro-3-(pyridin-3-ylethynyl)-N- |
| | (thiophen-2-ylmethyl)thieno[3,2-b]pyridin-7-amine trifluoroacetate |
| 309 | 5-(5-chloro-2-((2R,3R)-3-(methylamino)tetrahydro-2H-pyran-2-yl)-7- |
| | ((thiophen-2-ylmethyl)amino)thieno[3,2-b]pyridin-3-yl)pent-4-yn-1-ol |
| | trifluoroacetate and 5-(5-chloro-2-((2S,3S)-3-(methylamino)tetrahydro-2H- |
| | pyran-2-yl)-7-((thiophen-2-ylmethyl)amino)thieno[3,2-b]pyridin-3-yl)pent-4- |
| | yn-1-ol trifluoroacetate |
| 314 | 2-((3R,4S)-3-aminotetrahydro-2H-pyran-4-yl)-3,5-dichloro-N-(furan-2- |
| | ylmethyl)thieno[3,2-b]pyridin-7-amine trifluoroacetate and 2-((3S,4R)-3- |
| | aminotetrahydro-2H-pyran-4-yl)-3,5-dichloro-N-(furan-2-ylmethyl)thieno[3,2- |
| | b]pyridin-7-amine trifluoroacetate |
| 315 | 2-((3R,4S)-3-aminotetrahydro-2H-pyran-4-yl)-3,5-dichloro-N-(thiophen-2- |
| | ylmethyl)thieno[3,2-b]pyridin-7-amine trifluoroacetate and 2-((3S,4R)-3- |
| | aminotetrahydro-2H-pyran-4-yl)-3,5-dichloro-N-(thiophen-2- |
| | ylmethyl)thieno[3,2-b]pyridin-7-amine trifluoroacetate |
| 316 | 2-((3R,4S)-3-aminotetrahydro-2H-pyran-4-yl)-3-bromo-5-chloro-N-(furan-2- |
| | ylmethyl)thieno[3,2-b]pyridin-7-amine trifluoroacetate and 2-((3S,4R)-3- |
| | aminotetrahydro-2H-pyran-4-yl)-3-bromo-5-chloro-N-(furan-2- |
| | ylmethyl)thieno[3,2-b]pyridin-7-amine trifluoroacetate |
| 317 | 6-((3R,4S)-3-aminotetrahydro-2H-pyran-4-yl)-2-chloro-N-(furan-2-ylmethyl)- |
| | 7-((trimethylsilyl)ethynyl)thieno[3,2-d]pyrimidin-4-amine trifluoroacetate and |
| | 6-((3S,4R)-3-aminotetrahydro-2H-pyran-4-yl)-2-chloro-N-(furan-2-ylmethyl)- |
| | 7-((trimethylsilyl)ethynyl)thieno[3,2-d]pyrimidin-4-amine trifluoroacetate |
| 319 | 2-((3R,4S)-3-aminotetrahydro-2H-pyran-4-yl)-3,5-dichloro-N-(furan-2- |
| | ylmethyl)thieno[3,2-b]pyridin-7-amine trifluoroacetate and 2-((3S,4R)-3- |
| | aminotetrahydro-2H-pyran-4-yl)-3,5-dichloro-N-(furan-2-ylmethyl)thieno[3,2- |
| | b]pyridin-7-amine trifluoroacetate |
| 320 | 7-(2-((1S,6S)-6-aminocyclohex-3-en-1-yl)-5-chloro-7-((thiophen-2- |
| | ylmethyl)amino)thieno[3,2-b]pyridin-3-yl)hept-6-yn-1-ol formate |
| 322 | 2-((1S,6S)-6-aminocyclohex-3-en-1-yl-2,2,3,4,5,5-d6)-5-chloro-3-methyl-N- |
| | (phenylmethyl-d2)thieno[3,2-b]pyridin-7-amine trifluoroacetate |
| 324 | 2-((1R,6R)-6-aminocyclohex-3-en-1-yl)-5-chloro-3-(propa-1,2-dien-1-yl)-N- |
| | (thiophen-2-ylmethyl)thieno[3,2-b]pyridin-7-amine trifluoroacetate and 2- |
| | ((1S,6S)-6-aminocyclohex-3-en-1-yl)-5-chloro-3-(propa-1,2-dien-1-yl)-N- |
| | (thiophen-2-ylmethyl)thieno[3,2-b]pyridin-7-amine trifluoroacetate |
| 329 | (1R,5R,6S)-5-amino-6-(3-bromo-5-chloro-7-((thiophen-2- |
| | ylmethyl)amino)thieno[3,2-b]pyridin-2-yl)cyclohex-2-en-1-ol trifluoroacetate |
| | and (1S,5S,6R)-5-amino-6-(3-bromo-5-chloro-7-((thiophen-2- |
| | ylmethyl)amino)thieno[3,2-b]pyridin-2-yl)cyclohex-2-en-1-ol trifluoroacetate |
| 330 | 6-((3R,4S)-3-aminotetrahydro-2H-pyran-4-yl)-2-chloro-7-ethynyl-N-(furan-2- |
| | ylmethyl)thieno[3,2-d]pyrimidin-4-amine trifluoroacetate and 6-((3S,4R)-3- |
| | aminotetrahydro-2H-pyran-4-yl)-2-chloro-7-ethynyl-N-(furan-2- |
| | ylmethyl)thieno[3,2-d]pyrimidin-4-amine trifluoroacetate |
| 332 | 2-((1R,6R)-6-aminocyclohex-3-en-1-yl)-3-bromo-5-chloro-N-(cyclopent-1- |
| | en-1-ylmethyl)thieno[3,2-b]pyridin-7-amine formate and 2-((1S,6S)-6- |
| | aminocyclohex-3-en-1-yl)-3-bromo-5-chloro-N-(cyclopent-1-en-1- |
| | ylmethyl)thieno[3,2-b]pyridin-7-amine formate |
| 334 | 6-((1S,2S)-2-aminocyclohexyl)-2,7-dichloro-5-(difluoromethyl)-N-(furan-2- |
| | ylmethyl)-5H-pyrrolo[3,2-d]pyrimidin-4-amine formate and 6-((1R,2R)-2- |
| | aminocyclohexyl)-2,7-dichloro-5-(difluoromethyl)-N-(furan-2-ylmethyl)-5H- |
| | pyrrolo[3,2-d]pyrimidin-4-amine formate |
| 339 | 6-((3R,4S)-3-aminotetrahydro-2H-pyran-4-yl)-7-bromo-2-chloro-N-(furan-2- |
| | ylmethyl)thieno[3,2-d]pyrimidin-4-amine formate and 6-((3S,4R)-3- |
| | aminotetrahydro-2H-pyran-4-yl)-7-bromo-2-chloro-N-(furan-2- |
| | ylmethyl)thieno[3,2-d]pyrimidin-4-amine formate |
| 342 | 2-((1R,2S,3S)-2-amino-3-fluorocyclohexyl)-5-chloro-3-methyl-N-(thiophen- |
| | 2-ylmethyl)thieno[3,2-b]pyridin-7-amine trifluoroacetate and 2-((1S,2R,3R)- |
| | 2-amino-3-fluorocyclohexyl)-5-chloro-3-methyl-N-(thiophen-2- |
| | ylmethyl)thieno[3,2-b]pyridin-7-amine trifluoroacetate |
| 347 | 6-((3R,4S)-3-aminotetrahydro-2H-pyran-4-yl)-7-bromo-2-chloro-N-(thiophen- |
| | 2-ylmethyl)thieno[3,2-d]pyrimidin-4-amine formate and 6-((3S,4R)-3- |
| | aminotetrahydro-2H-pyran-4-yl)-7-bromo-2-chloro-N-(thiophen-2- |
| | ylmethyl)thieno[3,2-d]pyrimidin-4-amine formate |
| 350 | 2-((3R,4S)-3-aminotetrahydro-2H-pyran-4-yl)-5-chloro-N-(thiophen-2- |
| | ylmethyl)-3-vinylthieno[3,2-b]pyridin-7-amine formate and 2-((3S,4R)-3- |
| | aminotetrahydro-2H-pyran-4-yl)-5-chloro-N-(thiophen-2-ylmethyl)-3- |
| | vinylthieno[3,2-b]pyridin-7-amine formate |
| 354 | 6-((3R,4S)-3-aminotetrahydro-2H-pyran-4-yl)-2,7-dichloro-N-(furan-2- |
| | ylmethyl)thieno[3,2-d]pyrimidin-4-amine formate and 6-((3S,4R)-3- |
| | aminotetrahydro-2H-pyran-4-yl)-2,7-dichloro-N-(furan-2-ylmethyl)thieno[3,2- |
| | d]pyrimidin-4-amine formate |
| 355 | (1R,2S,3R)-3-amino-2-(3-bromo-5-chloro-7-((thiophen-2- |
| | ylmethyl)amino)thieno[3,2-b]pyridin-2-yl)cyclohexan-1-ol trifluoroacetate |
| | and (1S,2R,3S)-3-amino-2-(3-bromo-5-chloro-7-((thiophen-2- |
| | ylmethyl)amino)thieno[3,2-b]pyridin-2-yl)cyclohexan-1-ol trifluoroacetate |
| 359 | 2-((1R,6R)-6-amino-2,2-difluorocyclohexyl)-3-bromo-5-chloro-N-(thiophen- |
| | 2-ylmethyl)thieno[3,2-b]pyridin-7-amine trifluoroacetate and 2-((1S,6S)-6- |
| | amino-2,2-difluorocyclohexyl)-3-bromo-5-chloro-N-(thiophen-2- |
| | ylmethyl)thieno[3,2-b]pyridin-7-amine trifluoroacetate |
| 366 | methyl (2-((1S,6S)-6-aminocyclohex-3-en-1-yl)-3-bromo-5-chlorothieno[3,2- |
| | b]pyridin-7-yl)glycinate formate |
| 370 | 2-((1R,6S)-6-amino-2,2-difluorocyclohexyl)-3-bromo-N-(but-2-yn-1-yl)-5- |
| | chlorothieno[3,2-b]pyridin-7-amine trifluoroacetate and 2-((1S,6R)-6-amino- |
| | 2,2-difluorocyclohexyl)-3-bromo-N-(but-2-yn-1-yl)-5-chlorothieno[3,2- |
| | b]pyridin-7-amine trifluoroacetate |
| 371 | 2-((1R,5S,6S)-6-amino-5-methoxycyclohex-3-en-1-yl)-3-bromo-5-chloro-N- |
| | (thiophen-2-ylmethyl)thieno[3,2-b]pyridin-7-amine hydrochloride and 2- |
| | ((1S,5R,6R)-6-amino-5-methoxycyclohex-3-en-1-yl)-3-bromo-5-chloro-N- |
| | (thiophen-2-ylmethyl)thieno[3,2-b]pyridin-7-amine hydrochloride |
| 372 | 6-((1R,6R)-6-aminocyclohex-3-en-1-yl-2,2,3,4,5,5-d6)-7-bromo-2-chloro-N- |
| | (thiophen-2-ylmethyl)thieno[3,2-d]pyrimidin-4-amine trifluoroacetate |
| 374 | (1R,2S,3R)-2-amino-3-(5-chloro-3-methyl-7-((thiophen-2- |
| | ylmethyl)amino)thieno[3,2-b]pyridin-2-yl)cyclohexan-1-ol trifluoroacetate |
| | and |
| 375 | 3-bromo-5-chloro-2-((1R,2R)-2-(methylamino)cyclohexyl)-N-(thiophen-2- |
| | ylmethyl)thieno[3,2-b]pyridin-7-amine trifluoroacetate and 3-bromo-5-chloro- |
| | 2-((1S,2S)-2-(methylamino)cyclohexyl)-N-(thiophen-2-ylmethyl)thieno[3,2- |
| | b]pyridin-7-amine trifluoroacetate |
| 377 | (1R,5S,6R)-5-amino-6-(3,5-dichloro-7-((thiophen-2- |
| | ylmethyl)amino)thieno[3,2-b]pyridin-2-yl)cyclohex-2-en-1-ol formate |
| 378 | 2-((1S,6S)-6-aminocyclohex-3-en-1-yl)-N-((E)-but-2-en-1-yl)-5-chloro-3- |
| | methylthieno[3,2-b]pyridin-7-amine trifluoroacetate |
| 381 | 2-((3R,4S)-3-aminotetrahydro-2H-pyran-4-yl)-N-benzyl-3-bromo-5- |
| | chlorothieno[3,2-b]pyridin-7-amine trifluoroacetate and 2-((3S,4R)-3- |
| | aminotetrahydro-2H-pyran-4-yl)-N-benzyl-3-bromo-5-chlorothieno[3,2- |
| | b]pyridin-7-amine trifluoroacetate |
| 383 | 2-((1R,6S)-6-amino-2,2-difluorocyclohexyl)-N-(but-2-yn-1-yl)-5-chloro-3- |
| | iodothieno[3,2-b]pyridin-7-amine trifluoroacetate and 2-((1S,6R)-6-amino- |
| | 2,2-difluorocyclohexyl)-N-(but-2-yn-1-yl)-5-chloro-3-iodothieno[3,2- |
| | b]pyridin-7-amine trifluoroacetate |
| 387 | 5-chloro-3-iodo-2-((1R,6R)-6-(methylamino)cyclohex-3-en-1-yl)-N- |
| | (thiophen-2-ylmethyl)thieno[3,2-b]pyridin-7-amine formate and 5-chloro-3- |
| | iodo-2-((1S,6S)-6-(methylamino)cyclohex-3-en-1-yl)-N-(thiophen-2- |
| | ylmethyl)thieno[3,2-b]pyridin-7-amine formate |
| 388 | 2-((3R,4S)-3-aminotetrahydro-2H-pyran-4-yl)-N-benzyl-3,5- |
| | dichlorothieno[3,2-b]pyridin-7-amine trifluoroacetate and 2-((3S,4R)-3- |
| | aminotetrahydro-2H-pyran-4-yl)-N-benzyl-3,5-dichlorothieno[3,2-b]pyridin- |
| | 7-amine trifluoroacetate |
| 390 | 6-((3R,4S)-3-aminotetrahydro-2H-pyran-4-yl)-2-chloro-N-(furan-2-ylmethyl)- |
| | 7-vinylthieno[3,2-d]pyrimidin-4-amine formate and 6-((3S,4R)-3- |
| | aminotetrahydro-2H-pyran-4-yl)-2-chloro-N-(furan-2-ylmethyl)-7- |
| | vinylthieno[3,2-d]pyrimidin-4-amine formate |
| 393 | 2-((3R,4S)-3-aminotetrahydro-2H-pyran-4-yl)-5-chloro-7-((thiophen-2- |
| | ylmethyl)amino)thieno[3,2-b]pyridine-3-carbonitrile trifluoroacetate and 2- |
| | ((3S,4R)-3-aminotetrahydro-2H-pyran-4-yl)-5-chloro-7-((thiophen-2- |
| | ylmethyl)amino)thieno[3,2-b]pyridine-3-carbonitrile trifluoroacetate |
| 394 | 2-((1S,6S)-6-aminocyclohex-3-en-1-yl)-5-chloro-3-((2-methoxypyridin-3- |
| | yl)ethynyl)-N-(thiophen-2-ylmethyl)thieno[3,2-b]pyridin-7-amine |
| | trifluoroacetate |
| 395 | 2-((1S,6S)-6-aminocyclohex-3-en-1-yl)-5-chloro-3-(pyridin-4-ylethynyl)-N- |
| | (thiophen-2-ylmethyl)thieno[3,2-b]pyridin-7-amine trifluoroacetate |
| 400 | 2-((1S,2S)-2-aminocyclohexyl)-5-chloro-3-(fluoromethyl)-N-(thiophen-2- |
| | ylmethyl)thieno[3,2-b]pyridin-7-amine formate and 2-((1R,2R)-2- |
| | aminocyclohexyl)-5-chloro-3-(fluoromethyl)-N-(thiophen-2- |
| | ylmethyl)thieno[3,2-b]pyridin-7-amine formate |
| 410 | 2-((3S,4R)-3-aminotetrahydro-2H-pyran-4-yl)-3,5-dichloro-N-(furan-2- |
| | ylmethyl)thieno[3,2-b]pyridin-7-amine trifluoroacetate |
| 421 | 2-((1R,2R)-2-aminocyclohexyl)-3,5-dichloro-1-(difluoromethyl)-N-(thiophen- |
| | 2-ylmethyl)-1H-pyrrolo[3,2-b]pyridin-7-amine formate and 2-((1S,2S)-2- |
| | aminocyclohexyl)-3,5-dichloro-1 -(difluoromethyl)-N-(thiophen-2-ylmethyl)- |
| | 1H-pyrrolo[3,2-b]pyridin-7-amine formate |
| 425 | 2-((1R,6S)-6-amino-2,2-difluorocyclohexyl)-3-bromo-N-((E)-but-2-en-1-yl)- |
| | 5-chlorothieno[3,2-b]pyridin-7-amine trifluoroacetate and 2-((1S,6R)-6- |
| | amino-2,2-difluorocyclohexyl)-3-bromo-N-((E)-but-2-en-1-yl)-5- |
| | chlorothieno[3,2-b]pyridin-7-amine trifluoroacetate |
| 442 | 2-((1S,2S)-2-aminocyclohexyl)-N-((Z)-but-2-en-1-yl)-3,5-dichlorothieno[3,2- |
| | b]pyridin-7-amine trifluoroacetate |
| 447 | 2-((1S,6S)-6-aminocyclohex-3-en-1-yl)-5-chloro-3-((3-chloropyridin-4- |
| | yl)ethynyl)-N-(thiophen-2-ylmethyl)thieno[3,2-b]pyridin-7-amine |
| | trifluoroacetate |
| 448 | 2-((1S,6S)-6-aminocyclohex-3-en-1-yl)-5-chloro-N-(thiophen-2- |
| | ylmethyl)thieno[3,2-b]pyridin-7-amine trifluoroacetate |
| 449 | 6-((1R,6R)-6-aminocyclohex-3-en-1-yl-2,2,3,4,5,5-d6)-2-chloro-N-(furan-2- |
| | ylmethyl)-7-methylthieno[3,2-d]pyrimidin-4-amine trifluoroacetate |
| 454 | 5-(5-chloro-2-((1R,6R)-6-(dimethylamino)cyclohex-3-en-1-yl)-7-((thiophen- |
| | 2-ylmethyl)amino)thieno[3,2-b]pyridin-3-yl)pent-4-yn-1-ol formate and 5-(5- |
| | chloro-2-((1S,6S)-6-(dimethylamino)cyclohex-3-en-1-yl)-7-((thiophen-2- |
| | ylmethyl)amino)thieno[3,2-b]pyridin-3-yl)pent-4-yn-1-ol formate; |

wherein the form of the compound is a hydrate, solvate, racemate, enantiomer, diastereomer, stereoisomer, tautomer or isotope enriched form thereof.

The present application further provides a pharmaceutical composition comprising a compound provided herein, or a pharmaceutically acceptable salt thereof, and at least one pharmaceutically acceptable carrier.

The present application further provides a method of treating spinocerebellar ataxia type 3 (SCA3), the method comprising administering to the subject a therapeutically effective amount of a compound provided herein, or a pharmaceutically acceptable salt thereof.

One aspect of the method or use includes the compound of Formula (I) or a form thereof, wherein exon 4 skipping in the ATXN3 pre-mRNA is induced during the splicing process.

One aspect of the method or use includes the compound of Formula (I) or a form thereof, wherein levels of ATXN3 mRNA are decreased.

One aspect of the method or use includes the compound of Formula (I) or a form thereof, wherein ATXN3 protein is decreased.

One aspect of the present description relates to a pharmaceutical composition comprising a compound of Formula (I) or a form thereof and at least one pharmaceutically acceptable excipient for administering to a subject for the treatment of spinocerebellar ataxia type 3 (SCA3), also known as Machado-Joseph disease (MJD).

One aspect of the present description relates to the manufacture of a medicament for the treatment of spinocerebellar ataxia type 3 (SCA3), also known as Machado-Joseph disease (MJD), in a subject comprising a compound of Formula (I) or a form thereof and at least one pharmaceutically acceptable excipient.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used.

The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Chemical Definitions

The chemical terms used above and throughout the description herein, unless specifically defined otherwise, shall be understood by one of ordinary skill in the art to have the following indicated meanings.

As used herein, the term “C₁₋₆alkyl” generally refers to saturated hydrocarbon radicals having from one to eight carbon atoms in a straight or branched chain configuration, including, but not limited to, methyl, ethyl, n-propyl (also referred to as propyl or propanyl), isopropyl, n-butyl (also referred to as butyl or butanyl), isobutyl, sec-butyl, tert-butyl, n-pentyl (also referred to as pentyl or pentanyl), n-hexyl (also referred to as hexyl or hexanyl), and the like. In certain aspects, C₁₋₆alkyl includes, but is not limited to, C₁₋₆alkyl, C₁₋₄alkyl and the like. A C₁₋₆alkyl radical is optionally substituted with substituent species as described herein where allowed by available valences.

As used herein, the term “C₂₋₆alkenyl” generally refers to partially unsaturated hydrocarbon radicals having from two to eight carbon atoms in a straight or branched chain configuration and one or more carbon-carbon double bonds therein, including, but not limited to, ethenyl (also referred to as vinyl), allyl, propenyl and the like. In certain aspects, C₂₋₆alkenyl includes, but is not limited to, C₂₋₆alkenyl, C₂₋₄alkenyl and the like. A C₂₋₆alkenyl radical is optionally substituted with substituent species as described herein where allowed by available valences.

As used herein, the term “C₂₋₆alkynyl” generally refers to partially unsaturated hydrocarbon radicals having from two to eight carbon atoms in a straight or branched chain configuration and one or more carbon-carbon triple bonds therein, including, but not limited to, ethynyl (also referred to as acetylenyl), propynyl, butynyl and the like. In certain aspects, C₂₋₆alkynyl includes, but is not limited to, C₂₋₆alkynyl, C₂₋₄alkynyl and the like. A C₂₋₆alkynyl radical is optionally substituted with substituent species as described herein where allowed by available valences.

As used herein, the term “C₁₋₆alkoxy” generally refers to saturated hydrocarbon radicals having from one to eight carbon atoms in a straight or branched chain configuration of the formula: —O—C₁₋₆alkyl, including, but not limited to, methoxy, ethoxy, n-propoxy, isopropoxy, n-butoxy, isobutoxy, sec-butoxy, tert-butoxy, n-pentoxy, n-hexoxy and the like. In certain aspects, C₁₋₆alkoxy includes, but is not limited to, C₁₋₆alkoxy, C₁₋₄alkoxy and the like. A C₁₋₆alkoxy radical is optionally substituted with substituent species as described herein where allowed by available valences.

As used herein, the term “C₁₋₆alkoxy-C₂₋₆alkynyl” refers to a radical of the formula: —C₂₋₆alkynyl-O—C₁₋₆alkyl, wherein C₂₋₆alkynyl is partially or completely substituted with one or more C₁₋₆alkoxy radicals where allowed by available valences.

As used herein, the term “oxo” refers to a radical of the formula: ═O.

As used herein, the term “carboxyl” refers to a radical of the formula: —COOH, —C(O)OH or —CO₂H.

As used herein, the terms “C₁₋₆alkoxy-carbonyl” or “CO₂C₁₋₆alkyl” refer to a radical of the formula: —COO—C₁₋₆alkyl, —C(O)O—C₁₋₆alkyl or —CO₂—C₁₋₆alkyl.

As used herein, the term “carbamoyl” refers to a radical of the formula: —C(O)NH₂.

As used herein, the term “C₃₋₈cycloalkyl” generally refers to a saturated or partially unsaturated monocyclic, bicyclic or polycyclic hydrocarbon radical, including, but not limited to, cyclopropyl, cyclobutyl, cyclopentyl, cyclohexyl, cyclohexenyl, and the like. In certain aspects, C₃₋₈cycloalkyl includes, but is not limited to, C₃₋₈cycloalkyl, C₅₋₈cycloalkyl, and the like. A C₃₋₁₀cycloalkyl radical is optionally substituted with substituent species as described herein where allowed by available valences.

As used herein, the term “aryl” generally refers to a monocyclic, bicyclic or polycyclic aromatic carbon atom ring structure radical, including, but not limited to, phenyl, naphthyl, anthracenyl, fluorenyl, azulenyl, phenanthrenyl and the like. An aryl radical is optionally substituted with substituent species as described herein where allowed by available valences.

As used herein, the term “heteroaryl” generally refers to a monocyclic, bicyclic or polycyclic aromatic carbon atom ring structure radical in which one or more carbon atom ring members have been replaced, where allowed by structural stability, with one or more heteroatoms, such as an O, S or N atom, including, but not limited to, furanyl, thiophenyl, pyrrolyl, pyrazolyl, imidazolyl, isoxazolyl, isothiazolyl, oxazolyl, 1,3-thiazolyl, triazolyl, oxadiazolyl, thiadiazolyl, tetrazolyl, pyridinyl, pyrimidinyl, pyrazinyl, pyridazinyl, triazinyl, indolyl, indazolyl, indolizinyl, isoindolyl, benzofuranyl, benzothiophenyl, benzoimidazolyl, 1,3-benzothiazolyl, 1,3-benzoxazolyl, purinyl, quinolinyl, isoquinolinyl, quinazolinyl, quinoxalinyl and the like. A heteroaryl radical is optionally substituted on a carbon or nitrogen atom ring member with substituent species as described herein where allowed by available valences.

In certain aspects, the nomenclature for a heteroaryl radical may differ, such as in non-limiting examples where furanyl may also be referred to as furyl, thiophenyl may also be referred to as thienyl, pyridinyl may also be referred to as pyridyl, benzothiophenyl may also be referred to as benzothienyl and 1,3-benzoxazolyl may also be referred to as 1,3-benzooxazolyl.

In certain other aspects, the term for a heteroaryl radical may also include other regioisomers, such as in non-limiting examples where the term pyrrolyl may also include 2H-pyrrolyl, 3H-pyrrolyl and the like, the term pyrazolyl may also include 1H-pyrazolyl and the like, the term imidazolyl may also include 1H-imidazolyl and the like, the term triazolyl may also include 1H-1,2,3-triazolyl and the like, the term oxadiazolyl may also include 1,2,4-oxadiazolyl, 1,3,4-oxadiazolyl and the like, the term tetrazolyl may also include 1H-tetrazolyl, 2H-tetrazolyl and the like, the term indolyl may also include 1H-indolyl and the like, the term indazolyl may also include 1H-indazolyl, 2H-indazolyl and the like, the term benzoimidazolyl may also include 1H-benzoimidazolyl and the term purinyl may also include 9H-purinyl and the like.

As used herein, the term “heterocyclyl” generally refers to a saturated or partially unsaturated monocyclic, bicyclic or polycyclic carbon atom ring structure radical in which one or more carbon atom ring members have been replaced, where allowed by structural stability, with a heteroatom, such as an O, S or N atom, including, but not limited to, oxiranyl, oxetanyl, azetidinyl, tetrahydrofuranyl, pyrrolinyl, pyrrolidinyl, pyrazolinyl, pyrazolidinyl, imidazolinyl, imidazolidinyl, isoxazolinyl, isoxazolidinyl, isothiazolinyl, isothiazolidinyl, oxazolinyl, oxazolidinyl, thiazolinyl, thiazolidinyl, triazolinyl, triazolidinyl, oxadiazolinyl, oxadiazolidinyl, thiadiazolinyl, thiadiazolidinyl, tetrazolinyl, tetrazolidinyl, pyranyl, dihydro-2H-pyranyl, tetrahydropyranyl, thiopyranyl, 1,3-dioxanyl, 1,3-oxazinanyl, 1,2,5,6-tetrahydropyridinyl, 1,2,3,6-tetrahydropyridinyl, piperidinyl, piperazinyl, morpholinyl, thiomorpholinyl, 1,4-diazepanyl, 1,3-benzodioxolyl, 1,4-benzodioxanyl and the like. A heterocyclyl radical is optionally substituted on a carbon or nitrogen atom ring member with substituent species as described herein where allowed by available valences.

As used herein, the term “C₁₋₄alkyl-amino” refers to a radical of the formula: —NH—C₁₋₄alkyl.

As used herein, the term “halo-C₁₋₄alkyl-amino” refers to a radical of the formula: —NH—C₁₋₄alkyl, wherein C₁₋₄alkyl is partially or completely substituted with one or more halogen atoms where allowed by available valences.

As used herein, the term “(C₁₋₄alkyl)₂-amino” refers to a radical of the formula: —N(C₁₋₄alkyl)₂.

As used herein, the term “C₁₋₆alkyl-thio” refers to a radical of the formula: —S—C₁₋₆alkyl.

As used herein, the term “C₁₋₆alkyl-sulfoxyl” refers to a radical of the formula: —S(O)—C₁₋₆alkyl.

As used herein, the term “C₁₋₆alkyl-sulfonyl” refers to a radical of the formula: —SO₂—C₁₋₆alkyl.

As used herein, the term “halo” or “halogen” generally refers to a halogen atom radical, including fluoro, chloro, bromo, and iodo.

As used herein, the term “halo-C₁₋₆alkyl” refers to a radical of the formula: —C₁₋₆alkyl-halo, wherein C₁₋₆alkyl is partially or completely substituted with one or more halogen atoms where allowed by available valences.

As used herein, the term “halo-C₁₋₆alkoxy” refers to a radical of the formula: —O—C₁₋₆alkyl-halo, wherein C₁₋₆alkyl is partially or completely substituted with one or more halogen atoms where allowed by available valences.

As used herein, the term “deutero-C₁₋₆alkyl” refers to a radical of the formula: —C₁₋₆alkyl, wherein C₁₋₆alkyl is partially or completely substituted with one or more deuterium atoms where allowed by available valences.

As used herein, the term “hydroxy” refers to a radical of the formula: —OH.

As used herein, the term “hydroxy-C₁₋₆alkyl” refers to a radical of the formula: —C₁₋₆alkyl-OH, wherein C₁₋₆alkyl is partially or completely substituted with one or more hydroxy radicals where allowed by available valences.

As used herein, the term “hydroxy-C₂₋₆alkynyl” refers to a radical of the formula: —C₂₋₆alkynyl-OH, wherein C₂₋₆alkynyl is partially or completely substituted with one or more hydroxy radicals where allowed by available valences.

As used herein, the term “thio-C₁₋₆alkyl” refers to a radical of the formula: —C₁₋₆alkyl-SH, wherein C₁₋₆alkyl is partially or completely substituted with one or more —SH radicals where allowed by available valences.

As used herein, the term “C₁₋₆alkoxy-C₂₋₆alkynyl” refers to a radical of the formula —C₂₋₆alkynyl-C₁₋₆alkoxy, wherein C₂₋₆alkynyl is partially or completely substituted with one or more hydroxy radicals where allowed by available valences.

As used herein, the term “(CH₃)₃Si—C₂₋₆alkynyl” refers to a radical of the formula —C₂₋₆alkynyl-Si(CH₃)₃.

As used herein, the term “heteroaryl-C₂₋₆alkynyl” refers to a radical of the formula C₂₋₆alkynyl-heteroaryl.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances, one or more substituents having a double bond (e.g., “oxo” or “═O”) as the point of attachment may be described, shown or listed herein within a substituent group, wherein the structure may only show a single bond as the point of attachment to the core structure of Formula (I). A person of ordinary skill in the art would understand that, while only a single bond is shown, a double bond is intended for those substituents.

As used herein, the term “and the like,” with reference to the definitions of chemical terms provided herein, means that variations in chemical structures that could be expected by one skilled in the art include, without limitation, isomers (including chain, branching or positional structural isomers), hydration of ring systems (including saturation or partial unsaturation of monocyclic, bicyclic or polycyclic ring structures) and all other variations where allowed by available valences which result in a stable compound.

For the purposes of this description, where one or more substituent variables for a compound of Formula (I) or a form thereof encompass functionalities incorporated into a compound of Formula (I), each functionality appearing at any location within the disclosed compound may be independently selected, and as appropriate, independently and/or optionally substituted.

As used herein, the terms “independently selected,” or “each selected” refer to functional variables in a substituent list that may occur more than once on the structure of Formula (I), the pattern of substitution at each occurrence is independent of the pattern at any other occurrence. Further, the use of a generic substituent variable on any formula or structure for a compound described herein is understood to include the replacement of the generic substituent with species substituents that are included within the particular genus, e.g., aryl may be replaced with phenyl or naphthalenyl and the like, and that the resulting compound is to be included within the scope of the compounds described herein.

As used herein, the terms “each instance of” or “in each instance, when present,” when used preceding a phrase such as “ . . . C₃₋₁₀cycloalkyl, C₃₋₁₀cycloalkyl-C₁₋₄alkyl, aryl, aryl-C₁₋₄alkyl, heteroaryl, heteroaryl-C₁₋₄alkyl, heterocyclyl and heterocyclyl-C₁₋₄alkyl,” are intended to refer to the C₃₋₁₀cycloalkyl, aryl, heteroaryl and heterocyclyl ring systems when each are present either alone or as a substituent.

As used herein, the term “optionally substituted” means optional substitution with the specified substituent variables, groups, radicals or moieties.

As used herein, the term “D” refers to a deuterium atom.

As used herein, the term “isotope enriched” means a compounds of Formula I or a form thereof which are identical to those recited herein, but for the fact that one or more atoms are replaced by an atom having an atomic mass or mass number different from the atomic mass or mass number usually found in nature. Examples of isotopes that can be incorporated into compounds of Formula I or a form thereof described herein include isotopes of hydrogen, carbon, nitrogen, oxygen, phosphorus, fluorine and chlorine, such as H², H³, C¹³, C¹⁴, N¹⁵, O¹⁸, O¹⁷, P³¹, P³², S³⁵, F¹⁸, Cl³⁵ and Cl³⁶, respectively, each of which is also within the scope of this description.

### Compound Forms

As used herein, the term “form” means a compound of Formula (I) having a form selected from the group consisting of a free acid, free base, salt, hydrate, solvate, racemate, enantiomer, diastereomer, stereoisomer, and tautomer form thereof.

In certain aspects described herein, the form of the compound of Formula (I) is a free acid, free base or salt thereof.

In certain aspects described herein, the form of the compound of Formula (I) is a salt thereof.

In certain aspects described herein, the form of the compound of Formula (I) is a stereoisomer, racemate, enantiomer or diastereomer thereof.

In certain aspects described herein, the form of the compound of Formula (I) is a tautomer thereof.

In certain aspects described herein, the form of the compound of Formula (I) is a pharmaceutically acceptable form.

In certain aspects described herein, the compound of Formula (I) or a form thereof is isolated for use.

As used herein, the term “isolated” means the physical state of a compound of Formula (I) or a form thereof after being isolated and/or purified from a synthetic process (e.g., from a reaction mixture) or natural source or combination thereof according to an isolation or purification process or processes described herein or which are well known to the skilled artisan (e.g., chromatography, recrystallization and the like) in sufficient purity to be characterized by standard analytical techniques described herein or well known to the skilled artisan.

As used herein, the term “protected” means that a functional group in a compound of Formula (I) or a form thereof is in a form modified to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York. Such functional groups include hydroxy, phenol, amino and carboxylic acid. Suitable protecting groups for hydroxy or phenol include trialkylsilyl or diarylalkylsilyl (e.g., t-butyldimethylsilyl, t-butyldiphenylsilyl or trimethylsilyl), tetrahydropyranyl, benzyl, substituted benzyl, methyl, methoxymethanol, and the like. Suitable protecting groups for amino, amidino and guanidino include t-butoxycarbonyl, benzyloxycarbonyl, and the like. Suitable protecting groups for carboxylic acid include alkyl, aryl or arylalkyl esters. In certain instances, the protecting group may also be a polymer resin, such as a Wang resin or a 2-chlorotrityl-chloride resin. Protecting groups may be added or removed in accordance with standard techniques, which are well-known to those skilled in the art and as described herein. It will also be appreciated by those skilled in the art, although such protected derivatives of compounds described herein may not possess pharmacological activity as such, they may be administered to a subject and thereafter metabolized in the body to form compounds described herein which are pharmacologically active. Such derivatives may therefore be described as “prodrugs”. All prodrugs of compounds described herein are included within the scope of the use described herein.

One or more compounds described herein may exist in unsolvated as well as solvated forms with pharmaceutically acceptable solvents such as water, ethanol, and the like, and the description herein is intended to embrace both solvated and unsolvated forms.

As used herein, the term “solvate” means a physical association of a compound described herein with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. As used herein, “solvate” encompasses both solution-phase and isolatable solvates. Non-limiting examples of suitable solvates include ethanolates, methanolates, and the like.

As used herein, the term “hydrate” means a solvate wherein the solvent molecule is water.

The compounds of Formula (I) can form salts, which are intended to be included within the scope of this description. Reference to a compound of Formula (I) or a form thereof herein is understood to include reference to salt forms thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) or a form thereof contains both a basic moiety, such as, without limitation an amine moiety, and an acidic moiety, such as, but not limited to a carboxylic acid, zwitterions (“inner salts”) may be formed and are included within the term “salt(s)” as used herein.

The term “pharmaceutically acceptable salt(s)”, as used herein, means those salts of compounds described herein that are safe and effective (i.e., non-toxic, physiologically acceptable) for use in mammals and that possess biological activity, although other salts are also useful. Salts of the compounds of the Formula (I) may be formed, for example, by reacting a compound of Formula (I) or a form thereof with an amount of acid or base, such as an equivalent amount, in a medium such as one in which the salt precipitates or in an aqueous medium followed by lyophilization.

Pharmaceutically acceptable salts include one or more salts of acidic or basic groups present in compounds described herein. Particular aspects of acid addition salts include, and are not limited to, acetate, ascorbate, benzoate, benzenesulfonate, bisulfate, bitartrate, borate, bromide, butyrate, chloride, citrate, camphorate, camphorsulfonate, ethanesulfonate, formate, fumarate, gentisinate, gluconate, glucaronate, glutamate, iodide, isonicotinate, lactate, maleate, methanesulfonate, naphthalenesulfonate, nitrate, oxalate, pamoate, pantothenate, phosphate, propionate, saccharate, salicylate, succinate, sulfate, tartrate, thiocyanate, toluenesulfonate (also known as tosylate), trifluoroacetate salts and the like. Certain particular aspects of acid addition salts include chloride or dichloride.

Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33, 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Suitable basic salts include, but are not limited to, aluminum, ammonium, calcium, lithium, magnesium, potassium, sodium and zinc salts.

All such acid salts and base salts are intended to be included within the scope of pharmaceutically acceptable salts as described herein. In addition, all such acid and base salts are considered equivalent to the free forms of the corresponding compounds for purposes of this description.

Compounds of Formula (I) and forms thereof, may further exist in a tautomeric form. All such tautomeric forms are contemplated and intended to be included within the scope of the compounds of Formula (I) or a form thereof as described herein.

The compounds of Formula (I) or a form thereof may contain asymmetric or chiral centers, and, therefore, exist in different stereoisomeric forms. The present description is intended to include all stereoisomeric forms of the compounds of Formula (I) as well as mixtures thereof, including racemic mixtures.

The compounds described herein may include one or more chiral centers, and as such may exist as racemic mixtures (R/S) or as substantially pure enantiomers and diastereomers. The compounds may also exist as substantially pure (R) or (S) enantiomers (when one chiral center is present). In one particular aspect, the compounds described herein are (S) isomers and may exist as enantiomerically pure compositions substantially comprising only the (S) isomer. In another particular aspect, the compounds described herein are (R) isomers and may exist as enantiomerically pure compositions substantially comprising only the (R) isomer. As one of skill in the art will recognize, when more than one chiral center is present, the compounds described herein may also exist as a (R,R), (R,S), (S,R) or (S,S) isomer, as defined by IUPAC Nomenclature Recommendations.

As used herein, the term “chiral” refers to a carbon atom bonded to four nonidentical substituents. Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. In describing an optically active compound, the prefixes D and L, or R and S, are used to denote the absolute configuration of the molecule about its chiral center(s). The substituents attached to the chiral center under consideration are ranked in accordance with the Sequence Rule of Cahn, Ingold and Prelog. (Cahn et al. Angew. Chem. Inter. Edit. 1966, 5, 385; errata 511).

As used herein, the term “substantially pure” refers to compounds consisting substantially of a single isomer in an amount greater than or equal to 90%, in an amount greater than or equal to 92%, in an amount greater than or equal to 95%, in an amount greater than or equal to 98%, in an amount greater than or equal to 99%, or in an amount equal to 100% of the single isomer.

In one aspect of the description, a compound of Formula (I) or a form thereof is a substantially pure (S) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

In one aspect of the description, a compound of Formula (I) or a form thereof is a substantially pure (R) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

As used herein, a “racemate” is any mixture of isometric forms that are not “enantiomerically pure”, including mixtures such as, without limitation, in a ratio of about 50/50, about 60/40, about 70/30, or about 80/20.

In addition, the present description embraces all geometric and positional isomers. For example, if a compound of Formula (I) or a form thereof incorporates a double bond or a fused ring, both the cis- and trans-forms, as well as mixtures, are embraced within the scope of the description. Diastereomeric mixtures can be separated into their individual diastereomers on the basis of their physical chemical differences by methods well known to those skilled in the art, such as, for example, by chromatography and/or fractional crystallization. Enantiomers can be separated by use of chiral HPLC column or other chromatographic methods known to those skilled in the art. Enantiomers can also be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this description.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this description, as are positional isomers (such as, for example, 4-pyridyl and 3-pyridyl). Individual stereoisomers of the compounds described herein may, for example, be substantially free of other isomers, or may be present in a racemic mixture, as described supra.

The use of the terms “salt”, “solvate” and the like, is intended to equally apply to enantiomers, stereoisomers, rotamers, tautomers, positional isomers, or racemates of the instant compounds.

Chemical names were obtained using the structure naming feature of ChemDraw® software version 20.1.1 provided by PerkinElmer Informatics, Inc. and generally comport with the IUPAC (International Union for Pure and Applied Chemistry) recommendations on Nomenclature of Organic Chemistry and the CAS Index rules. The chemical names may have only parentheses or may have parentheses and brackets. The stereochemical descriptors may also be placed at different locations within the name itself, depending on the naming convention. One of ordinary skill in the art will recognize these formatting variations and understand they provide the same chemical structure.

### Compound Uses

Provided herein are methods of treating a disease in a subject in need thereof. As used herein, the terms “subject” or “patient” refer to any animal, including mammals. For example, mice, rats, other rodents, rabbits, dogs, cats, swine, cattle, sheep, horses, primates, and humans. In some aspects, the subject is a human.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician. In some aspects, the dosage of the compound, or a pharmaceutically acceptable salt thereof, administered to a subject or individual is about 1 mg to about 2 g, about 1 mg to about 1000 mg, about 1 mg to about 500 mg, about 1 mg to about 100 mg, about 1 mg to 50 mg, or about 50 mg to about 500 mg.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder (i.e., arresting further development of the pathology and/or symptomatology); and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

The present application provides a method of treating SCA3 in a subject in need thereof, comprising administering to the subject a therapeutically effective amount of a compound provided herein (i.e., a compound of Formula (I)).

Also provided herein is a method of treating a subject having a disease caused by abnormal repeat expansions in the ATXN3 gene which results in mutant ATXN3 protein possessing a polyQ expansion, comprising administering to the subject a therapeutically effective amount of a compound provided herein (i.e., a compound of Formula (I)).

Also provided herein are methods of lowering ATXN3 mutant protein in a subject, comprising administering to the subject a therapeutically effective amount of a compound provided herein (i.e., a compound of Formula (I)).

In some aspects of the methods provided herein, the compound is selected from the group of compounds of Formula (I) or a pharmaceutically acceptable salt thereof.

Also provided herein are methods of inducing exon skipping in mutant ATXN3 pre-mRNA in a subject, comprising administering to a subject an effective amount of a compound of Formula (I) or form thereof.

Also provided herein are methods of inducing exon skipping in mutant ATXN3 pre-mRNA in a cell, comprising contacting a cell (e.g. ex vivo or in vivo) with a compound of Formula (I) or form thereof.

Also provided herein are methods of inducing exon skipping in mutant ATXN3 pre-mRNA in a gene comprising contacting the gene (e.g., in a cell or subject expressing the gene) with a compound a compound of Formula (I) or a form thereof.

Also provided therein are methods of inducing exon 4 skipping in the mutant ATXN3 pre-mRNA in a subject in need thereof, the method comprising administering an effective amount of a compound Formula (I) or a form thereof to the subject.

Also provided therein are methods of inducing exon 4 skipping in the mutant ATXN3 pre-mRNA in a cell, the method comprising contacting the cell (e.g. ex vivo or in vivo) with a compound Formula (I) or a form thereof to the subject.

Also provided herein are methods of inducing exon 4 skipping in the mutant ATXN3 pre-mRNA in a gene comprising contacting the gene (e.g., in a cell or subject expressing the gene) with a compound a compound of Formula (I) or a form thereof.

Also provided therein are methods of producing ATXN3 ΔE4 in a subject in need thereof, the method comprising administering an effective amount of a compound Formula (I) or a form thereof to the subject.

Also provided therein are methods of producing ATXN3 ΔE4 in a cell, the method comprising contacting the cell (e.g. ex vivo or in vivo) with a compound Formula (I) or a form thereof to the subject.

Also provided herein are methods of producing ATXN3 ΔE4 in a gene comprising contacting the gene (e.g., in a cell or subject expressing the gene) with a compound a compound of Formula (I) or a form thereof.

Also provided herein are methods for decreasing mutant ATXN3 mRNA in a subject in need thereof, the method comprising administering an effective amount of a compound of Formula (I) or a form thereof to the subject. For example, such methods include decreasing mutant ATXN3 mRNA concentration in serum samples from the subject.

In some aspects, mutant ATXN3 mRNA can be measured in the serum, for example, in blood samples obtained from the subject prior to administration of a compound of Formula (I) or form thereof and in blood samples obtained from the subject following administration of a compound as provided herein. In some aspects, the blood samples obtained from the subject following administration are obtained after one day, two days, three days, four days, five days, six days, seven days, eight days, nine days, ten days, fourteen days, twenty-one days, twenty-eight days, and/or thirty days of administration of the compound as provided herein. See, for example, F. B. Axelrod et al., Pediatr Res (2011) 70(5): 480-483; and R. S. Shetty et al., Human Molecular Genetics (2011) 20(21): 4093-4101, both of which are incorporated by reference in their entirety.

Further provided herein is a method for decreasing mutant ATXN3 mRNA in a cell, the method comprising contacting the cell (e.g. ex vivo or in vivo) with a therapeutically effective amount of a compound of Formula (I) or a form salt thereof. The amount of mutant ATXN3 mRNA in the treated cell is decreased relative to a cell in a subject in the absence of a compound provided herein. The method for decreasing the amount of mutant ATXN3 mRNA in a cell may be performed by contacting the cell with a compound of Formula (I) or a form thereof in vitro, thereby decreasing the amount of mutant ATXN3 mRNA of a cell in vitro. Uses of such an in vitro method of decreasing the amount of mutant ATXN3 mRNA include, but are not limited to, use in a screening assay (for example, wherein a compound of Formula (I) or a form thereof is used as a positive control or standard compared to a compound or compounds of unknown activity or potency in decreasing the amount mutant ATXN3 mRNA).

In some aspects, the amount of mutant ATXN3 mRNA is decreased in a cell selected from the group consisting of a lung cell, a muscle cell, a liver cell, a heart cell, a brain cell, a kidney cell, a spleen cell, and a nerve cell (e.g., a sciatic nerve cell or a trigeminal nerve cell), or any combination thereof. In some aspects thereof, the amount of mutant ATXN3 mRNA is decreased in the plasma.

The method of decreasing mutant ATXN3 mRNA in a cell may be performed, for example, by contacting a cell, (e.g., a lung cell, a muscle cell, a liver cell, a heart cell, a brain cell, a kidney cell, a spleen cell, or a nerve cell), with a compound of Formula (I) or a form thereof in vivo, thereby decreasing the amount of mutant ATXN3 mRNA in a subject in vivo. The contacting is achieved by causing a compound of Formula (I) or a form thereof to be present in a subject in an amount effective to achieve a decrease in the amount of mutant ATXN3 mRNA. This may be achieved, for example, by administering an effective amount of a compound of Formula (I) or a form thereof to a subject. Uses of such an in vivo method of decreasing the amount of mutant ATXN3 mRNA include, but are not limited to, use in methods of treating a disease or condition, wherein a decrease in the amount of mutant ATXN3 mRNA is beneficial.

In some aspects thereof, the amount of mutant ATXN3 mRNA is decreased in a cell selected from the group consisting of a lung cell, a muscle cell, a liver cell, a heart cell, a brain cell, a kidney cell, a spleen cell, and a nerve cell (e.g., a sciatic nerve cell or a trigeminal nerve cell), or any combination thereof, for example in a subject suffering from SCA3. The method is preferably performed by administering an effective amount of a compound of Formula (I) or a form thereof to a subject who is suffering from SCA3.

Also provided herein are methods for decreasing ATXN3 mutant protein expression in a subject in need thereof, the method comprising administering an effective amount of a compound of Formula (I) or a pharmaceutically acceptable salt thereof to the subject. For example, such methods include decreasing ATXN3 mutant protein expression in serum samples from the subject. Further provided herein are methods for decreasing the mean percentage of ATXN3 mutant protein expression in a subject in need thereof, the method comprising administering an effective amount of a compound of Formula (I) or a form thereof to the subject.

Also provided herein are methods for decreasing ATXN3 mutant protein expression in a cell (e.g., ex vivo or in vivo), the method comprising contacting the cell with a therapeutically effective amount of a compound of Formula (I) or a form thereof. In some aspects the method is an in vitro method. In some aspects, the method is an in vivo method. In some aspects, the amount ATXN3 mutant protein expression is decreased in a cell selected from the group consisting of a lung cell, a muscle cell, a liver cell, a heart cell, a brain cell, a kidney cell, a spleen cell, and a nerve cell (e.g., a sciatic nerve cell or a trigeminal nerve cell), or any combination thereof. In some aspects thereof, the amount of ATXN3 mutant protein expression is decreased in the plasma.

Also provided herein are methods for decreasing ATXN3 mutant protein level in a subject in need thereof, the method comprising administering an effective amount of a compound of Formula (I) or a form thereof to the subject. For example, such methods include decreasing ATXN3 mutant protein level in serum samples from the subject. Further provided herein are methods for decreasing the mean percentage of ATXN3 mutant protein level in a subject in need thereof, the method comprising administering an effective amount of a compound of Formula (I) or a form thereof, to the subject.

Also provided herein are methods for decreasing ATXN3 mutant protein level in a cell (e.g., ex vivo or in vivo), the method comprising contacting the cell with a therapeutically effective amount of a compound of Formula (I) or a form thereof.

In some aspects, the method is an in vitro method. In some aspects, the method is an in vivo method. In some aspects, the amount of ATXN3 mutant protein level is decreased in a cell selected from the group consisting of a lung cell, a muscle cell, a liver cell, a heart cell, a brain cell, a kidney cell, a spleen cell, and a nerve cell (e.g., a sciatic nerve cell or a trigeminal nerve cell), or any combination thereof. In some aspects thereof, the amount of ATXN3 mutant protein level is decreased in plasma.

In some aspects, one or more of the compounds of Formula (I) or form thereof may be administered to a subject in need thereof in combination with at least one additional pharmaceutical agent.

Additional examples of suitable additional pharmaceutical agents for use in combination with the compounds of the present application for treatment of the diseases provided herein include, but are not limited to, antioxidants, anti-inflammatory agents, steroids, immunosuppressants, or other agents such as therapeutic antibodies. In some aspects, the compounds of Formula (I) or a form thereof may be administered to a subject in need thereof in combination with at least one additional pharmaceutical agent for the treatment of SCA3.

When employed as a therapeutic agent, the compounds provided herein can be administered in the form of a pharmaceutical composition; thus, the methods described herein can include administering a pharmaceutical composition. These compositions can be prepared as described herein or elsewhere, and can be administered by a variety of routes, depending upon whether local or systemic treatment is desired and upon the area to be treated. Administration may be pulmonary (e.g., by inhalation or insufflation of powders or aerosols, including by nebulizer; intratracheal or intranasal), oral, or parenteral. Parenteral administration may include, but is not limited to intravenous, intraarterial, subcutaneous, intraperitoneal, intramuscular injection or infusion; or intracranial, (e.g., intrathecal, intraocular, or intraventricular) administration. Parenteral administration can be in the form of a single bolus dose, or may be, for example, by a continuous perfusion pump. Conventional pharmaceutical carriers, aqueous, powder or oily bases, thickeners and the like may be necessary or desirable. In some aspects, the compounds provided herein are suitable for oral and parenteral administration. In some aspects, the compounds provided herein are suitable for oral administration. In some aspects, the compounds provided herein are suitable for parenteral administration. In some aspects, the compounds provided herein are suitable for intravenous administration. In some aspects, the compounds provided herein are suitable for transdermal administration (e.g., administration using a patch or microneedle). Pharmaceutical compositions for topical administration may include transdermal patches (e.g., normal or electrostimulated), ointments, lotions, creams, gels, drops, suppositories, sprays, liquids and powders. Conventional pharmaceutical carriers, aqueous, powder or oily bases, thickeners and the like may be necessary or desirable.

Also provided are pharmaceutical compositions which contain, as the active ingredient, a compound of Formula (I) or a form thereof in combination with one or more pharmaceutically acceptable carriers (excipients). In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil; wetting agents; emulsifying and suspending agents; preserving agents such as methyl- and propylhydroxy-benzoates; sweetening agents; flavoring agents, or combinations thereof.

The active compound can be effective over a wide dosage range and is generally administered in a pharmaceutically effective amount. It will be understood that the amount of compound to be administered and the schedule of administration will usually be determined by a physician, according to the relevant circumstances, including the condition to be treated, the chosen route of administration, the actual compound administered, the age, weight, and response of the individual subject, the severity of the subject's symptoms, and the like.

In another aspect, the concentration-biological effect relationship observed with regard to a compound of Formula (I) or a form thereof indicate a target plasma concentration ranging from approximately 0.001 μg·hr/mL to approximately 50 μg·hr/mL, from approximately 0.01 μg·hr/mL to approximately 20 μg·hr/mL, from approximately 0.05 μg·hr/mL to approximately 10 μg·hr/mL, or from approximately 0.1 μg·hr/mL to approximately 5 μg·hr/mL. To achieve such plasma concentrations, the compounds described herein may be administered at doses that vary, such as, for example, without limitation, from 1.0 ng to 10,000 mg.

In one aspect, the dose administered to achieve an effective target plasma concentration may be administered based upon subject or patient specific factors, wherein the doses administered on a weight basis may be in the range of from about 0.001 mg/kg/day to about 3500 mg/kg/day, or about 0.001 mg/kg/day to about 3000 mg/kg/day, or about 0.001 mg/kg/day to about 2500 mg/kg/day, or about 0.001 mg/kg/day to about 2000 mg/kg/day, or about 0.001 mg/kg/day to about 1500 mg/kg/day, or about 0.001 mg/kg/day to about 1000 mg/kg/day, or about 0.001 mg/kg/day to about 500 mg/kg/day, or about 0.001 mg/kg/day to about 250 mg/kg/day, or about 0.001 mg/kg/day to about 200 mg/kg/day, or about 0.001 mg/kg/day to about 150 mg/kg/day, or about 0.001 mg/kg/day to about 100 mg/kg/day, or about 0.001 mg/kg/day to about 75 mg/kg/day, or about 0.001 mg/kg/day to about 50 mg/kg/day, or about 0.001 mg/kg/day to about 25 mg/kg/day, or about 0.001 mg/kg/day to about 10 mg/kg/day, or about 0.001 mg/kg/day to about 5 mg/kg/day, or about 0.001 mg/kg/day to about 1 mg/kg/day, or about 0.001 mg/kg/day to about 0.5 mg/kg/day, or about 0.001 mg/kg/day to about 0.1 mg/kg/day, or from about 0.01 mg/kg/day to about 3500 mg/kg/day, or about 0.01 mg/kg/day to about 3000 mg/kg/day, or about 0.01 mg/kg/day to about 2500 mg/kg/day, or about 0.01 mg/kg/day to about 2000 mg/kg/day, or about 0.01 mg/kg/day to about 1500 mg/kg/day, or about 0.01 mg/kg/day to about 1000 mg/kg/day, or about 0.01 mg/kg/day to about 500 mg/kg/day, or about 0.01 mg/kg/day to about 250 mg/kg/day, or about 0.01 mg/kg/day to about 200 mg/kg/day, or about 0.01 mg/kg/day to about 150 mg/kg/day, or about 0.01 mg/kg/day to about 100 mg/kg/day, or about 0.01 mg/kg/day to about 75 mg/kg/day, or about 0.01 mg/kg/day to about 50 mg/kg/day, or about 0.01 mg/kg/day to about 25 mg/kg/day, or about 0.01 mg/kg/day to about 10 mg/kg/day, or about 0.01 mg/kg/day to about 5 mg/kg/day, or about 0.01 mg/kg/day to about 1 mg/kg/day, or about 0.01 mg/kg/day to about 0.5 mg/kg/day, or about 0.01 mg/kg/day to about 0.1 mg/kg/day, or from about 0.1 mg/kg/day to about 3500 mg/kg/day, or about 0.1 mg/kg/day to about 3000 mg/kg/day, or about 0.1 mg/kg/day to about 2500 mg/kg/day, or about 0.1 mg/kg/day to about 2000 mg/kg/day, or about 0.1 mg/kg/day to about 1500 mg/kg/day, or about 0.1 mg/kg/day to about 1000 mg/kg/day, or about 0.1 mg/kg/day to about 500 mg/kg/day, or about 0.1 mg/kg/day to about 250 mg/kg/day, or about 0.1 mg/kg/day to about 200 mg/kg/day, or about 0.1 mg/kg/day to about 150 mg/kg/day, or about 0.1 mg/kg/day to about 100 mg/kg/day, or about 0.1 mg/kg/day to about 75 mg/kg/day, or about 0.1 mg/kg/day to about 50 mg/kg/day, or about 0.1 mg/kg/day to about 25 mg/kg/day, or about 0.1 mg/kg/day to about 10 mg/kg/day, or about 0.1 mg/kg/day to about 5 mg/kg/day, or about 0.1 mg/kg/day to about 1 mg/kg/day, or about 0.1 mg/kg/day to about 0.5 mg/kg/day.

Effective amounts for a given subject may be determined by routine experimentation that is within the skill and judgment of a clinician or a practitioner skilled in the art in light of factors related to the subject. Dosage and administration may be adjusted to provide sufficient levels of the active agent(s) or to maintain the desired effect. Factors which may be taken into account include genetic screening, severity of the disease state, status of disease progression, general health of the subject, ethnicity, age, weight, gender, diet, time of day and frequency of administration, drug combination(s), reaction sensitivities, experience with other therapies, and tolerance/response to therapy.

The dose administered to achieve an effective target plasma concentration may be orally administered once (once in approximately a 24 hour period; i.e., “q.d.”), twice (once in approximately a 12 hour period; i.e., “b.i.d.” or “q.12 h”), thrice (once in approximately an 8 hour period; i.e., “t.i.d.” or “q.8 h”), or four times (once in approximately a 6 hour period; i.e., “q.d.s.”, “q.i.d.” or “q.6 h”) daily.

In certain aspects, the dose administered to achieve an effective target plasma concentration may also be administered in a single, divided, or continuous dose for a patient or subject having a weight in a range of between about 40 to about 200 kg (which dose may be adjusted for patients or subjects above or below this range, particularly children under 40 kg). The typical adult subject is expected to have a median weight in a range of about 70 kg. Long-acting pharmaceutical compositions may be administered every 2, 3 or 4 days, once every week, or once every two weeks depending on half-life and clearance rate of the particular formulation.

The compounds and compositions described herein may be administered to the subject via any drug delivery route known in the art. Nonlimiting examples include oral, ocular, rectal, buccal, topical, nasal, sublingual, transdermal, subcutaneous, intramuscular, intraveneous (bolus and infusion), intracerebral, and pulmonary routes of administration.

In another aspect, the dose administered may be adjusted based upon a dosage form described herein formulated for delivery at about 0.02, 0.025, 0.03, 0.05, 0.06, 0.075, 0.08, 0.09, 0.10, 0.20, 0.25, 0.30, 0.50, 0.60, 0.75, 0.80, 0.90, 1.0, 1.10, 1.20, 1.25, 1.50, 1.75, 2.0, 3.0, 5.0, 10, 20, 30, 40, 50, 100, 150, 200, 250, 300, 400, 500, 1000, 1500, 2000, 2500, 3000 or 4000 mg/day.

For any compound, the effective amount can be estimated initially either in cell culture assays or in relevant animal models, such as a mouse, guinea pig, chimpanzee, marmoset or tamarin animal model. Relevant animal models may also be used to determine the appropriate concentration range and route of administration. Such information can then be used to determine useful doses and routes for administration in humans. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is therapeutic index, and can be expressed as the ratio, LD₅₀/ED₅₀. In certain aspects, the effective amount is such that a large therapeutic index is achieved. In further particular aspects, the dosage is within a range of circulating concentrations that include an ED₅₀ with little or no toxicity. The dosage may vary within this range depending upon the dosage form employed, sensitivity of the patient, and the route of administration.

Another aspect included within the scope of the present description are the use of in vivo metabolic products of the compounds described herein. Such products may result, for example, from the oxidation, reduction, hydrolysis, amidation, esterification and the like of the administered compound, primarily due to enzymatic processes. Accordingly, the description includes the use of compounds produced by a process comprising contacting a compound described herein with a mammalian tissue or a mammal for a period of time sufficient to yield a metabolic product thereof.

Such products typically are identified by preparing a radio-labeled (e.g., ¹⁴C or ³H) compound of Formula (I), administering the radio-labeled compound in a detectable dose (e.g., greater than about 0.5 mg/kg) to a mammal such as a rat, mouse, guinea pig, dog, monkey or human, allowing sufficient time for metabolism to occur (typically about 30 seconds to about 30 hours), and identifying the metabolic conversion products from urine, bile, blood or other biological samples. The conversion products are easily isolated since they are “radiolabeled” by virtue of being isotopically-enriched (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well-known to those skilled in the art. The conversion products, so long as they are not otherwise found in vivo, are useful in diagnostic assays for therapeutic dosing of the compounds described herein even if they possess no biological activity of their own.

### Compounds and Preparation

Examples of representative compounds encompassed by the present invention and within the scope of the invention are provided herein. These examples and preparations which follow are provided to enable those skilled in the art to more clearly understand and to practice the present invention. They should not be considered as limiting the scope of the invention, but merely as being illustrative and representative thereof.

### General Synthetic Examples

As disclosed herein, the methods for preparing the compounds of Formula (I) or a form thereof described herein commonly use standard, well-known synthetic methodology. Many of the starting materials are commercially available or can be prepared in the Specific Synthetic Examples that follow using techniques known to those skilled in the art. Functional transformations to modify substituents may also be undertaken where chemically feasible and are considered to be included within the scope of the General Schemes and the knowledge of a person of ordinary skill in the art. Compounds of Formula (I) or a form thereof can be prepared as described in the Schemes below. Depending on the nature of the groups depicted in the schemes below, the final compounds or their precursors may be further elaborated using the standard, well-known synthetic methods.

### Scheme A:

Compounds of Formula (I) may be prepared as described in Scheme A below.

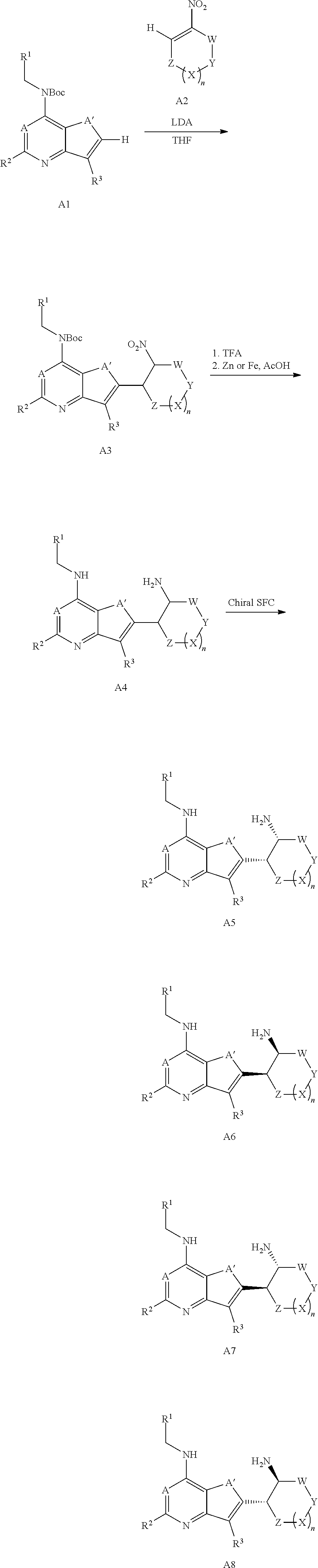

Compound A1 with A and A′ atoms as well as optionally substituted R¹, R², and R³ substituents can be reacted with cyclic nitroolefin A2 in the presence of a strong base (such as LDA and the like) in a suitable solvent (such as THF and the like) at an appropriate temperature such as −78° C. to give A3. Deprotection may be accomplished by treatment with an acid (such as HCl in dioxane or TFA and the like) followed by reduction using Zn or Fe metal in the presence of acid (such as acetic acid and the like) to deliver A4. Chiral SFC may be used to separate individual diastereomers A5, A6, A7, and A8.

### Scheme B:

Compounds of Formula (I) may be prepared as described in Scheme B below.

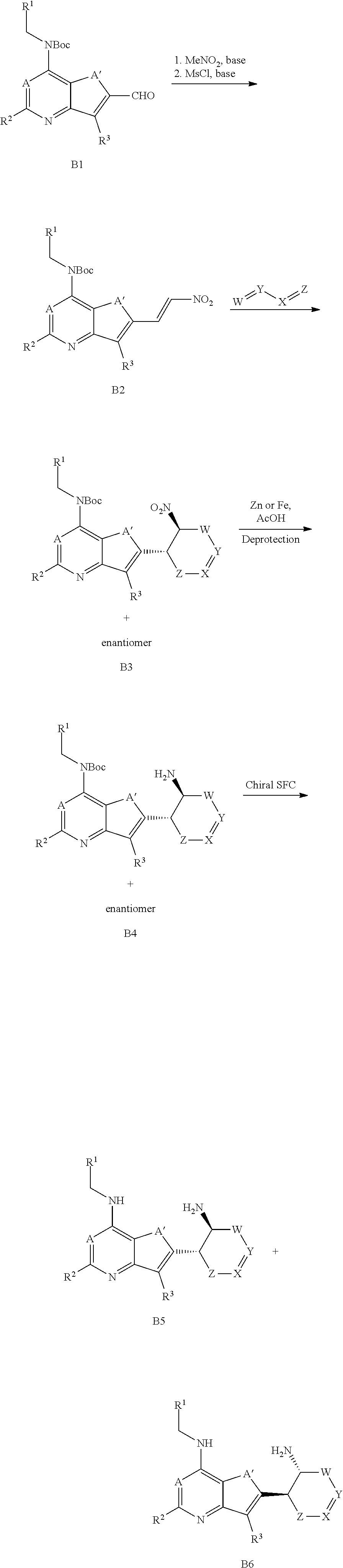

Compound B1 with A and A′ atoms as well as optionally substituted R¹, R², and R³ substituents can be reacted with nitromethane is the presence of base (such as NaOH and the like) followed by dehydration with MsCl in the presence of a suitable base (such as TEA and the like) gives nitroolefin B2. Diels-Alder reaction with an optionally substituted olefin in a suitable solvent (such as toluene and the like) at an appropriate temperature (such as 100° C.) affords B3. Reduction with Zn or Fe metal in the presence of acid (such as AcOH and the like) followed by protecting group deprotection if necessary provides B4. Chiral SFC separation provides B5 and B6.

### Scheme C:

Compounds of Formula (I) may be prepared as described in Scheme C below.

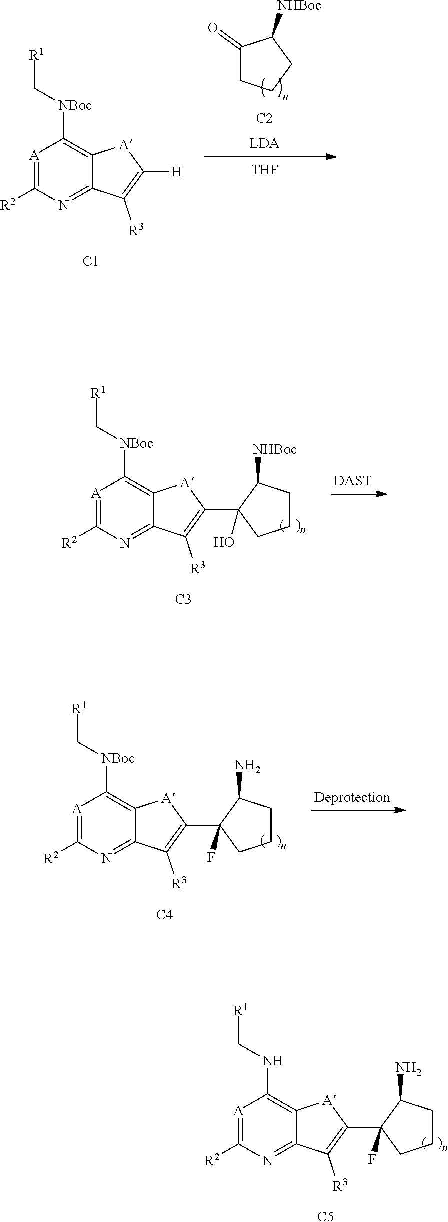

Compound Cl with A and A′ atoms as well as optionally substituted R¹, R², and R³ substituents can be reacted with α-amino ketone C2 in the presence of a strong base (such as LDA and the like) in a suitable solvent (such as THF and the like) at an appropriate temperature such as −78° C. to give C3. Treatment with a deoxyfluorination reagent (such as DAST and the like) in a suitable solvent (such as DCM and the like) affords C4. Deprotection may be accomplished by treatment with an acid (such as HCl in dioxane or TFA and the like) to deliver C5.

### Scheme D:

Compounds of Formula (I) may be prepared as described in Scheme D below.

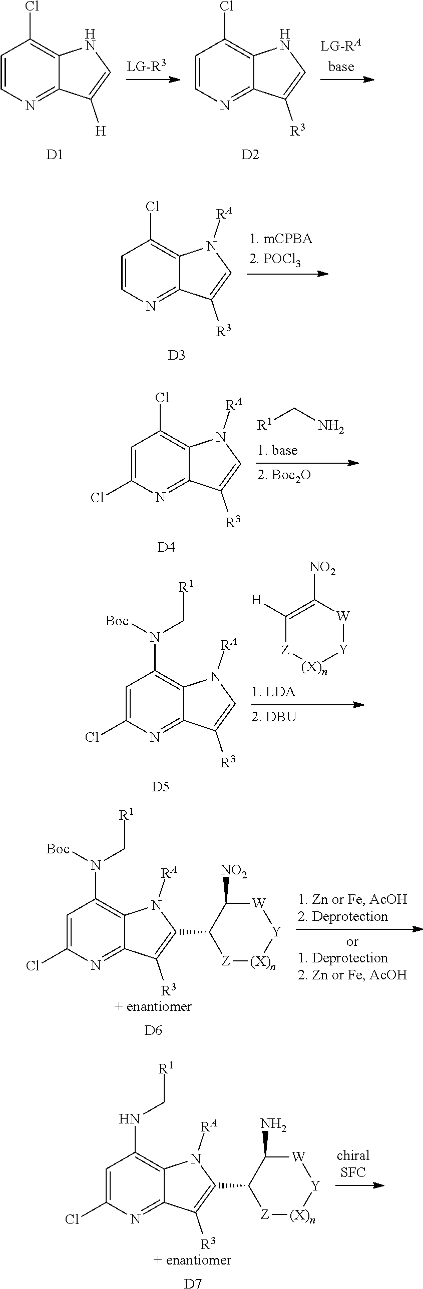

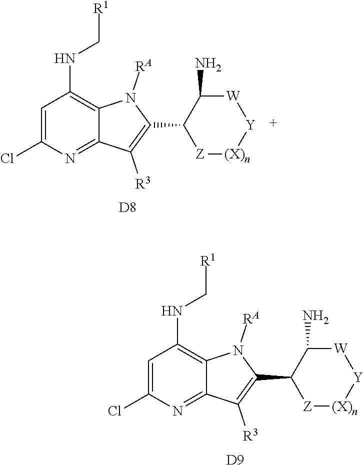

Azaindole D1 can be reacted with an appropriate electrophile (such as NBS and the like) to deliver optionally substituted D2. Optionally substituted azaindole D2 can be treated with an appropriate electrophile (such as Mel and the like) in the presence of a suitable base (such as Cs₂CO₃ and the like) to afford D3. Reaction with an appropriate oxidant (such as mCPBA and the like) followed by treatment with a deoxychlorination reagent (such as POCl₃ and the like) at an appropriate temperature (such as 110° C.) provides chloropyridine D4. Compound D4 can be treated with an optionally substituted aryl/heteroarylmethylamine in the presence of a base (such as TEA and the like) using a suitable solvent (such as DMSO and the like) at an appropriate temperature followed by protection with Boc₂O in the presence of base (such as TEA and the like) to afford intermediate D5. Compound D5 with optionally substituted R¹, R^{A}, and R³ substituents can be reacted with a cyclic nitroolefin in the presence of a strong base (such as LDA and the like) in a suitable solvent (such as THF and the like) at an appropriate temperature such as −78° C. followed by treatment with an appropriate base (such as DBU and the like) to give D6. Deprotection may be accomplished by treatment with an acid (such as HCl in dioxane or TFA and the like) followed by reduction using Zn or Fe metal in the presence of acid (such as acetic acid and the like) delivers D7. Chiral SFC may be used to separate individual enantiomers D8 and D9.

### Scheme E:

Compounds of Formula (I) may be prepared as described in Scheme E below.

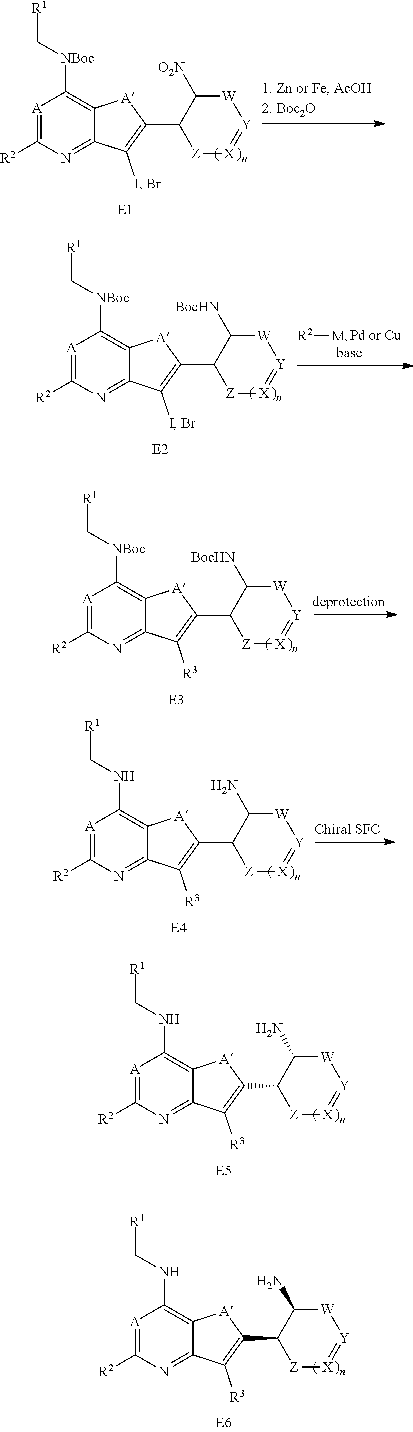

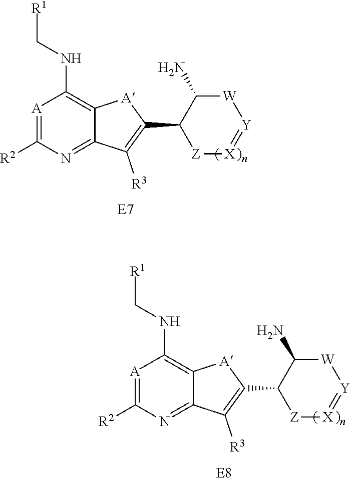

Reduction of E1, having A and A′ atoms as well as optionally substituted R¹, R², and R³ substituents, with Zn or Fe metal in the presence of an appropriate acid (such as AcOH or the like) followed by reaction with Boc₂O delivers E2. Compound E2 may be converted into E3 through reaction with an appropriately functionalized organometallic species in the presence of a catalyst (such as Pd(dppf)Cl₂ and the like) and a base (such as TEA and the like). Compound E3 may be deprotected with an appropriate acid (TFA or HCl and the like) to afford E4. Chiral SFC may then be used to separate the individual diastereomers E5, E6, E7, and E8.

### Specific Synthetic Examples

To describe in more detail and assist in understanding, the following non-limiting examples are offered to more fully illustrate the scope of compounds described herein and are not to be construed as specifically limiting the scope thereof. Such variations of the compounds described herein that may be now known or later developed, which would be within the purview of one skilled in the art to ascertain, are considered to fall within the scope of the compounds as described herein and hereinafter claimed. These examples illustrate the preparation of certain compounds. Those of skill in the art will understand that the techniques described in these examples represent techniques, as described by those of ordinary skill in the art, that function well in synthetic practice, and as such constitute preferred modes for the practice thereof. However, it should be appreciated that those of skill in the art should, in light of the present disclosure, appreciate that many changes can be made in the specific methods that are disclosed and still obtain a like or similar result without departing from the spirit and scope of the present description.

Other than in the following examples of the embodied compounds, unless indicated to the contrary, all numbers expressing quantities of ingredients, reaction conditions, experimental data, and so forth used in the specification and claims are to be understood as being modified by the term “about”. Accordingly, all such numbers represent approximations that may vary depending upon the desired properties sought to be obtained by a reaction or as a result of variable experimental conditions. Therefore, within an expected range of experimental reproducibility, the term “about” in the context of the resulting data, refers to a range for data provided that may vary according to a standard deviation from the mean. As well, for experimental results provided, the resulting data may be rounded up or down to present data consistently, without loss of significant figures. At the very least, and not as an attempt to limit the application of the doctrine of equivalents to the scope of the claims, each numerical parameter should be construed in light of the number of significant digits and rounding techniques used by those of skill in the art.

While the numerical ranges and parameters setting forth the broad scope of the present description are approximations, the numerical values set forth in the examples set forth below are reported as precisely as possible. Any numerical value, however, inherently contains certain errors necessarily resulting from the standard deviation found in their respective testing measurements.

### Compound Examples

As used above, and throughout the present description, the following abbreviations, unless otherwise indicated, shall be understood to have the following meanings:

| Abbreviation | Meaning |
|---|---|
| Δ | heating (chemistry), deletion (biology), lack of (biology) |
| AcOH or HOAc | acetic acid |
| Ar | argon |
| ACN | acetonitrile |
| atm | atmosphere(s) |
| BHT | butylated hydroxytoluene |
| Boc | tert-butoxy-carbonyl |
| ° C. | degrees Celsius |
| Celite ® or Celite | diatomaceous earth |
| d | day(s) |
| DAST | diethylaminosulfur trifluoride |
| DBU | 1,8-diazabicyclo[5.4.0]undec-7-ene |
| DCM | dichloromethane |
| IDEA or DIPEA | N,N-diisopropylethylamine |
| DMAP | 4-dimethylaminopyridine |
| DMP | Dess-Martin periodinane |
| DMF | dimethylformamide |
| DMSO | dimethylsulfoxide |
| DPPA | diphenylphosphoryl azide |
| EA or EtOAc | ethyl acetate |
| EtOH | ethanol |
| eq. | equivalent(s) |
| h or hr | hour(s) |
| HATU | 1-[bis(dimethylamino)methylene]-1H-1,2,3-triazolo[4,5- |
| | b]pyridinium 3-oxid hexafluorophosphate, N- |
| | [(dimethylamino)-1H-1,2,3-triazolo-[4,5-b]pyridin-1- |
| | ylmethylene]-N-methylmethanaminium |
| | hexafluorophosphate N-oxide |
| hex | hexanes |
| HPLC | High-performance liquid chromatography |
| LC/MS, LCMS or | liquid chromatographic mass spectroscopy |
| LC-MS | |
| LDA | lithium diisopropylamide |
| M | molarity |
| min | minute(s) |
| MeOH | methanol |
| MS | mass spectroscopy |
| NMR | nuclear magnetic resonance |
| Pd(dppf)Cl₂ or | [1,1′- |
| Pd(dppf)Cl₂-CH₂Cl₂ | bis(diphenylphosphino)ferrocene]dichloropalladium(II), |
| | complex with dichloromethane |
| PhMe or PhCH₃ | toluene |
| PE | petroleum ether |
| psi | pounds per square inch pressure |
| PTFE | polytetrafluoroethylene |
| Rt or rt | room temperature |
| RT | retention time |
| s or sec | second(s) |
| SFC | supercritical fluid chromatography |
| TBAF | tetrabutylammonium fluoride |
| TBS | tert-butyldimethylsilyl |
| TEA, Et₃N or NEt₃ | triethylamine |
| TFA | trifluoroacetic acid |
| Tf₂O | trifluoromethanesulfonic anhydride |
| THF | tetrahydrofuran |
| TLC | thin layer chromatography |
| TMS | trimethylsilyl |
| UPLCMS | ultra-performance liquid chromatography-mass |
| | spectrometry |

### Intermediate 1

### tert-Butyl (3-bromo-5-chloro-2-formylthieno[3,2-b]pyridin-7-yl)(thiophen-2-ylmethyl)carbamate

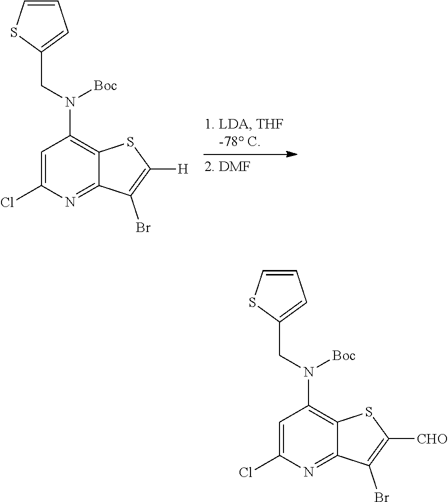 [SMILES: CC(C)(C)OC(=O)N(Cc1cccs1)c1cc(Cl)nc2c(Br)csc12 ; CC(C)(C)OC(=O)N(Cc1cccs1)c1cc(Cl)nc2c(Br)c(C=O)sc12]

### Step 1: tert-Butyl (3-bromo-5-chloro-2-formylthieno[3,2-b]pyridin-7-yl)(thiophen-2-ylmethyl)carbamate

To a solution of tert-butyl (3-bromo-5-chlorothieno[3,2-b]pyridin-7-yl)(thiophen-2-ylmethyl)carbamate (0.50 g, 1.087 mmol, 1.0 eq.), in tetrahydrofuran (10 mL) at −78° C. was added lithium diisopropylamide (0.82 mL, 1.64 mmol, 1.5 eq.). This solution stirred for 30 minutes followed by the addition of dimethylformamide (0.24 mg, 3.2 mmol, 3 eq.). The reaction stirred at −78° C. for one hour followed by warming to room temperature and continued stirring for 1 hour. The reaction was then quenched with aqueous ammonium chloride. The mixture was extracted with ethyl acetate and the organics were washed with water and brine. The organic layer was dried over Na₂SO₄, filtered, and concentrated in vacuo. The residue was purified by silica gel column chromatography with ethyl acetate in hexanes (0 to 50% gradient) to give tert-butyl (3-bromo-5-chloro-2-formylthieno[3,2-b]pyridin-7-yl)(thiophen-2-ylmethyl)carbamate (340 mg, 65% yield). MS m/z 485.9, 486.6 [M+H]⁺; ¹H NMR (400 MHz, CHLOROFORM-d) δ ppm 10.26 (s, 1H), 7.19-7.14 (m, 2H), 6.81 (dd, J=5.1, 3.4 Hz, 1H), 6.74 (dd, J=3.4, 1.1 Hz, 1H), 4.99 (s, 2H), 1.41 (s, 9H).

### Intermediate 2

### tert-Butyl (5-chloro-3-ethylthieno[3,2-b]pyridin-7-yl)(thiophen-2-ylmethyl)carbamate

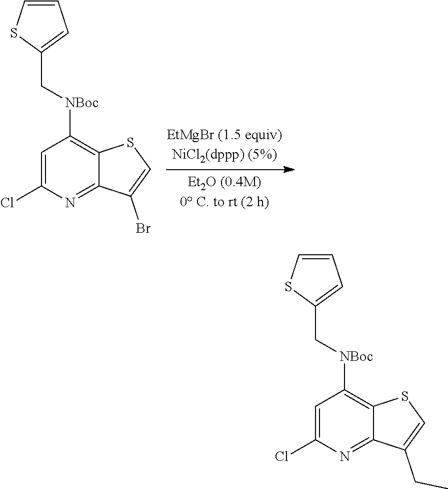 [SMILES: CC(C)(C)OC(=O)N(Cc1cccs1)c1cc(Cl)nc2c(Br)csc12 ; CCc1csc2c(N(Cc3cccs3)C(=O)OC(C)(C)C)cc(Cl)nc12]

### Step 1: tert-Butyl (5-chloro-3-ethylthieno[3,2-b]pyridin-7-yl)(thiophen-2-ylmethyl)carbamate

To an 8-mL vial capped with a septum, t-butyl (3-bromo-5-chlorothieno[3,2-b]pyridin-7-yl)(thiophen-2-ylmethyl)carbamate (200 mg, 0.43 mmol) and [1,3-bis(diphenylphosphino)propane]nickel(II)chloride (12 mg, 0.022 mmol, 0.05 equiv) were added. The vial was evacuated and back-filled with argon 3 times, and then anhydrous diethyl ether (1 mL) was added. The reaction was placed in an ice bath and allowed to stir, under argon, for 15 minutes, at which point ethylmagnesium bromide (1M) in THF (0.7 mL, 0.7 mmol, 1.5 equiv) was added dropwise via syringe. The ice bath was removed shortly thereafter, and the reaction was allowed to stir at room temperature, under argon, for 2 hours. The reaction mixture was then transferred to separatory funnel, adding ethyl acetate (20 mL) and saturated aqueous ammonium chloride (20 mL). The layers were separated, the aqueous layer was extracted twice with ethyl acetate (20 mL), and the combined organic layers were dried over Na₂SO₄, filtered, and concentrated in vacuo. The crude reaction mixture was purified by silica gel chromatography (40 g SiO₂, 0% to 10% ethyl acetate in hexanes) to cleanly obtain 85 mg (48% yield) of t-butyl (5-chloro-3-ethylthieno[3,2-b]pyridin-7-yl)(thiophen-2-ylmethyl)carbamate as a white solid after lyophilization. MS m/z 408.95 [M+H]⁺, ¹H NMR (DMSO-d₆) δ ppm 7.83 (s, 1H), 7.44 (s, 1H), 7.39-7.42 (m, 1H), 6.82-6.89 (m, 2H), 5.11 (s, 2H), 2.81 (q, J=7.46 Hz, 2H), 1.39 (s, 9H), 1.26 (t, J=7.50 Hz, 3H).

### Intermediate 3

### tert-Butyl (5-chloro-3-iodothieno[3,2-b]pyridin-7-yl)(thiophen-2-ylmethyl)carbamate

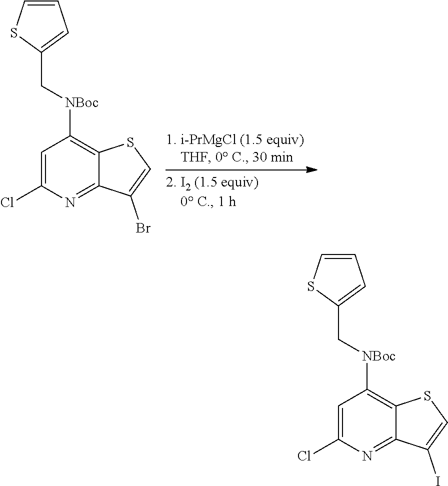 [SMILES: CC(C)(C)OC(=O)N(Cc1cccs1)c1cc(Cl)nc2c(Br)csc12 ; CC(C)(C)OC(=O)N(Cc1cccs1)c1cc(Cl)nc2c(I)csc12]

### Step 1: tert-Butyl (5-chloro-3-iodothieno[3,2-b]pyridin-7-yl)(thiophen-2-ylmethyl)carbamate

t-Butyl (3-bromo-5-chlorothieno[3,2-b]pyridin-7-yl)(thiophen-2-ylmethyl)carbamate (200 mg, 0.43 mmol) was added to a 50-mL round-bottom flask, and iodine (166 mg, 0.65 mmol, 1.5 equiv) was added to a 25-mL conical flask. Both flasks underwent 3 cycles of being briefly placed under high vacuum then back-filled with argon. Anhydrous THF (2.2 mL, degassed with argon directly prior to use) was added to each flask, and the resultant solutions were allowed to stir under an argon flow. Both flasks were placed in an ice bath. After about 15 minutes, isopropylmagnesium chloride (2M solution in THF, Acros SureSeal, 0.33 mL, 1.5 equiv) was slowly added to the flask containing the aryl bromide. The reaction, which quickly turned dark reddish-brown, was allowed to stir at 0° C. for 30 minutes, at which point the cold solution of iodine in THF was added via cannula transfer. The reaction was allowed to stir at 0° C. for 1 hour, at which point saturated aqueous ammonium chloride (20 mL) and ethyl acetate (20 mL) were added. The mixture was allowed to stir briefly, open to air, and was then transferred to a separatory funnel. The layers were separated, and the aqueous layer was then extracted 2 times with ethyl acetate (20 mL). The combined organic layers were washed twice with saturated aqueous sodium thiosulfate (40 mL) and once with brine (40 mL), then dried over Na₂SO₄, filtered, and concentrated in vacuo. The crude material was purified by silica gel chromatography (40 g SiO₂, 0% to 10% ethyl acetate gradient in hexanes). The purified residue was lyophilized to obtain 155 mg (70% yield) of t-butyl (5-chloro-3-iodothieno[3,2-b]pyridin-7-yl)(thiophen-2-ylmethyl)carbamate as a white solid. MS m/z 506.7 [M+H]⁺, ¹H NMR (DMSO-d₆) δ ppm 8.44 (s, 1H), 7.60 (s, 1H), 7.37-7.42 (m, 1H), 6.83-6.89 (m, 2H), 5.13 (s, 2H), 1.39 (s, 9H).

### Intermediate 4

### tert-Butyl (2,7-dichloro-5-(difluoromethyl)-5H-pyrrolo[3,2-d]pyrimidin-4-yl)(furan-2-ylmethyl)carbamate

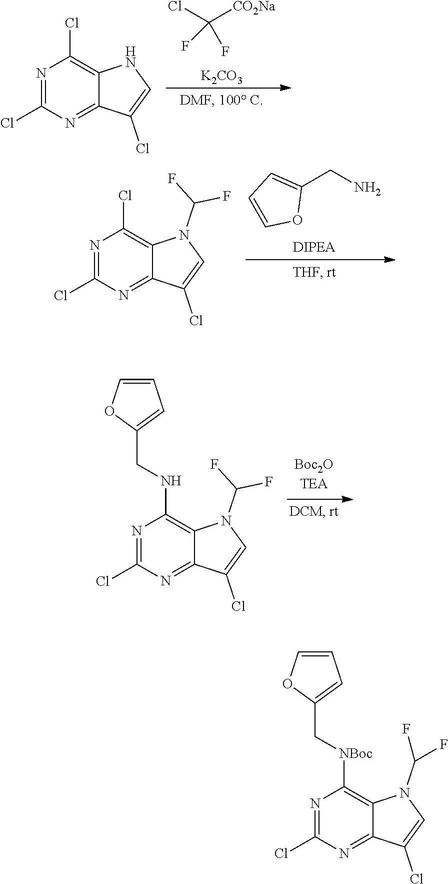

### Step 1: 2,4,7-Trichloro-5-(difluoromethyl)-5H-pyrrolo[3,2-d]pyrimidine

To a mixture of 2,4,7-trichloro-5H-pyrrolo[3,2-d]pyrimidine (10.0 g, 45 mmol, 1.0 eq.) in dimethylformamide (100 mL) at room temperature was added sodium chlorodifluoroacetate (8.2 g, 54 mmol, 1.2 eq.) and potassium carbonate (12.4 g, 90 mmol, 2.0 eq.). After stirring at 100° C. for 20 min, the reaction mixture was cooled to room temperature and poured into water (300 mL). The aqueous phase was extracted with EtOAc (3×200 mL) and the combined organics were washed with brine (300 mL), dried over Na₂SO₄, filtered, and concentrated. The crude residue was purified by silica gel column chromatography (PE/EA 20:1 to 10:1 gradient) to afford 2,4,7-trichloro-5-(difluoromethyl)-5H-pyrrolo[3,2-d]pyrimidine (9.3 g, 75%) as a white solid. MS m/z 271.8 [M+H]⁺.

### Step 2: 2,7-Dichloro-5-(difluoromethyl)-N-(furan-2-ylmethyl)-5H-pyrrolo[3,2-d]pyrimidin-4-amine

To a solution of 2,4,7-trichloro-5-(difluoromethyl)-5H-pyrrolo[3,2-d]pyrimidine (1.0 g, 3.7 mmol, 1.0 eq.) in tetrahydrofuran (15 mL) was added N,N-diisopropylethylamine (2.6 mL, 15 mmol, 4 eq.) and 2-furylmethanamine (0.4 g, 4 mmol, 1.1 eq.). After stirring at room temperature for 2 h, the reaction mixture was diluted with EtOAc (100 mL) and washed with water (100 mL), brine (100 mL), dried over Na2SO4, filtered, and concentrated. The crude residue was purified by silica gel chromatography (PE/EA 50:1) to afford 2,7-dichloro-5-(difluoromethyl)-N-(furan-2-ylmethyl)-5H-pyrrolo[3,2-d]pyrimidin-4-amine (1.1 g, 89%) as a yellow solid. MS m/z 332.9 [M+H]⁺.

### Step 3: tert-Butyl (2,7-dichloro-5-(difluoromethyl)-5H-pyrrolo[3,2-d]pyrimidin-4-yl)(furan-2-ylmethyl)carbamate

To a solution of 2,7-dichloro-5-(difluoromethyl)-N-(furan-2-ylmethyl)-5H-pyrrolo[3,2-d]pyrimidin-4-amine (1.1 g, 3.3 mmol, 1 eq.) in tetrahydrofuran (20 mL) was added (Boc)2O (1.5 g, 6.9 mmol, 2.1 eq), TEA (0.7 g, 7 mmol, 2.1 eq.) and DMAP (50 mg, 0.4 mmol, 0.1 eq.). After stirring at room temperature for 1 h, the reaction mixture was concentrated and the crude residue was purified by silica gel chromatography (EA/PE 1:10) to afford tert-butyl (2,7-dichloro-5-(difluoromethyl)-5H-pyrrolo[3,2-d]pyrimidin-4-yl)(furan-2-ylmethyl)carbamate (1.0 g, 70%) as a yellow solid. MS m/z 434.3 [M+H]⁺; ¹HNMR (400 MHz, CHLOROFORM-d) S: 7.82 (s, 1H), 7.26 (d, J=6.6 Hz, 2H), 6.26-6.16 (m, 2H), 5.3-5.16 (m, 2H), 1.38 (s, 9H).

### Intermediates 5 and 6

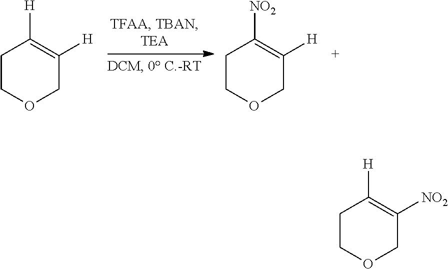 [SMILES: C1=CCOCC1 ; O=[N+]([O-])C1=CCOCC1 ; O=[N+]([O-])C1=CCCOC1]

### 4-Nitro-3,6-dihydro-2H-pyran; 5-nitro-3,6-dihydro-2H-pyran

To a solution of 3,6-dihydro-2H-pyran (12.5 g, 149 mmol, 1.0 eq.) in dichloromethane (250 mL), was added tetrabutylammonium nitrate (49.8 g, 164 mmol, 1.1 eq.) at room temperature. The solution was then cooled to 0° C. and trifluoroacetic anhydride (34.3 g, 163 mmol, 1.1 eq.) was added dropwise over 20 minutes. After stirring at room temperature for 2 h, the reaction mixture was again cooled to 0° C. and triethylamine (15.0 g, 149 mmol, 1.0 eq.) was added and stirred for an additional hour. The reaction mixture was diluted with DCM (100 mL) and washed with ice water (2×150 mL) followed by brine (2×150 mL). The organics were dried over Na₂SO₄, filtered, and concentrated under reduced pressure. The crude residue was purified by silica gel column chromatography (EA/PE, 0-10% gradient) to afford 4-nitro-3,6-dihydro-2H-pyran (500 mg, 3%) as a yellow oil and 5-nitro-3,6-dihydro-2H-pyran (1.8 g, 10%) as a yellow oil.

4-Nitro-3,6-dihydro-2H-pyran: ¹H NMR (400 MHz, CHLOROFORM-d) δ: 7.31-7.27 (m, 1H), 4.40 (q, J=2.9 Hz, 2H), 3.88 (t, J=5.5 Hz, 2H), 2.71 (ddd, J=5.4, 4.2, 2.6 Hz, 2H).

5-Nitro-3,6-dihydro-2H-pyran: ¹H NMR (400 MHz, CHLOROFORM-d) δ: 7.40 (t, J=4.2 Hz, 1H), 4.54 (dd, J=4.3, 2.6 Hz, 2H), 3.78 (t, J=5.4 Hz, 2H), 2.54-2.42 (m, 2H).

### Intermediate 7

### 3,3-Difluoro-1-nitrocyclohex-1-ene

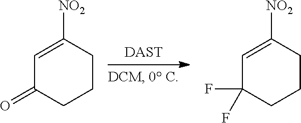

### 3,3-Difluoro-1-nitrocyclohex-1-ene

To a solution of 3-nitrocyclohex-2-en-1-one (1 g, 7 mmol) in DCM (7 mL), cooled to 0° C., was added diethylaminosulfur trifluoride (3.43 g, 21 mmol, 3 eq.). The mixture was stirred at rt for 16 h then slowly poured a cooled bath of saturated aqueous NaHCO₃. The aqueous phase was extracted with EtOAc (2×100 mL) and the combined organics were dried over Na₂SO₄, filtered, and concentrated in vacuo. The crude material was purified by silica gel column chromatography (EA/PE 1:20) to afford 3,3-difluoro-1-nitrocyclohex-1-ene (600 mg, 52%) as a yellow oil. ¹H NMR (400 MHz, CHLOROFORM-d) δ: 7.04 (t, J=5.4 Hz, 1H), 2.74-2.65 (m, 2H), 2.18-2.08 (m, 2H), 2.03-1.96 (m, 2H).

### Intermediate 8

### tert-Butyl (3-bromo-5-chloro-1-methyl-1H-pyrrolo[3,2-b]pyridin-7-yl)(thiophen-2-ylmethyl)carbamate

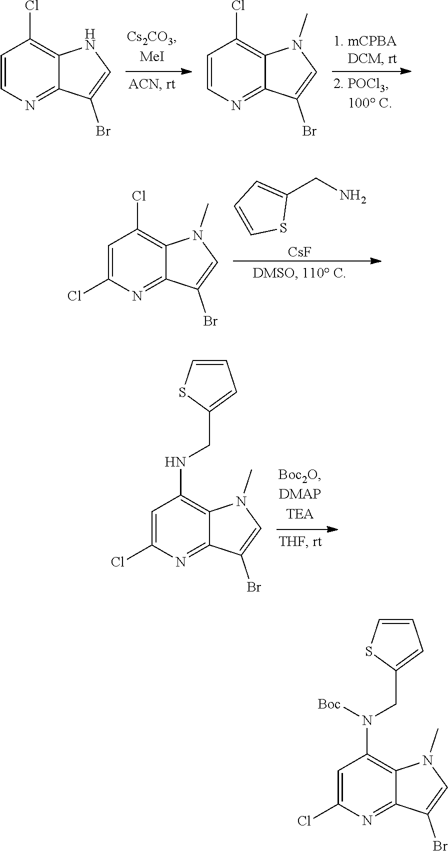

### Step 1: 3-Bromo-7-chloro-1-methyl-1H-pyrrolo[3,2-b]pyridine

To a solution of 3-bromo-7-chloro-1H-pyrrolo[3,2-b]pyridine (9.5 g, 41 mmol) in DMF (95 mL) was added Cs₂CO₃ (26.7 g, 82 mmol, 2 eq.) and iodomethane (8.74 g, 62 mmol, 1.5 eq.). After stirring at room temperature for 2 h, the reaction mixture was diluted with water (200 mL) and extracted with EtOAc (200 mL×2). The combined organics were washed with brine (200 mL×2), dried over Na₂SO₄, filtered, and concentrated in vacuo to afford 3-bromo-7-chloro-1-methyl-1H-pyrrolo[3,2-b]pyridine (7 g, 69%) as yellow solid. MS m/z 246.8 [M+H]⁺. ¹H NMR (400 MHz, CHLOROFORM-d) δ: 8.42 (d, J=5.1 Hz, 1H), 7.34 (s, 1H), 7.20 (d, J=5.1 Hz, 1H), 4.15 (s, 3H).

### Step 2: 3-Bromo-5,7-dichloro-1-methyl-1H-pyrrolo[3,2-b]pyridine

The title compound was prepared from 3-bromo-7-chloro-1-methyl-1H-pyrrolo[3,2-b]pyridine according to the procedure described in Intermediate 9, step 3. MS m/z 281.0 [M+H]⁺. ¹H NMR (400 MHz, CHLOROFORM-d) S: 7.30 (s, 1H), 7.18 (s, 1H), 4.11 (s, 3H).

### Step 3: 3-Bromo-5-chloro-1-methyl-N-(thiophen-2-ylmethyl)-1H-pyrrolo[3,2-b]pyridin-7-amine

The title compound was prepared from 3-bromo-7-chloro-1-methyl-1H-pyrrolo[3,2-b]pyridine according to the procedure described in Intermediate 9, step 4. MS m/z 358.0 [M+H]⁺.

### Step 4: tert-Butyl (3-bromo-5-chloro-1-methyl-1H-pyrrolo[3,2-b]pyridin-7-yl)(thiophen-2-ylmethyl)carbamate

The title compound was prepared from 3-bromo-7-chloro-1-methyl-1H-pyrrolo[3,2-b]pyridine according to the procedure described in Intermediate 9, step 5. MS m/z 458.1 [M+H]⁺. ¹H NMR (400 MHz, CHLOROFORM-d) S: 7.24 (d, J=5.0 Hz, 1H), 7.20 (s, 1H), 6.91 (dd, J=4.9, 3.3 Hz, 2H), 6.81 (s, 1H), 4.98 (d, J=7.9 Hz, 2H), 3.50 (s, 3H), 1.50 (s, 9H).

### Intermediate 9

### 2-((2S,3S)-3-Aminotetrahydro-2H-pyran-2-yl)-3-bromo-5-chloro-1-(difluoromethyl)-N-(thiophen-2-ylmethyl)-1H-pyrrolo[3,2-b]pyridin-7-amine

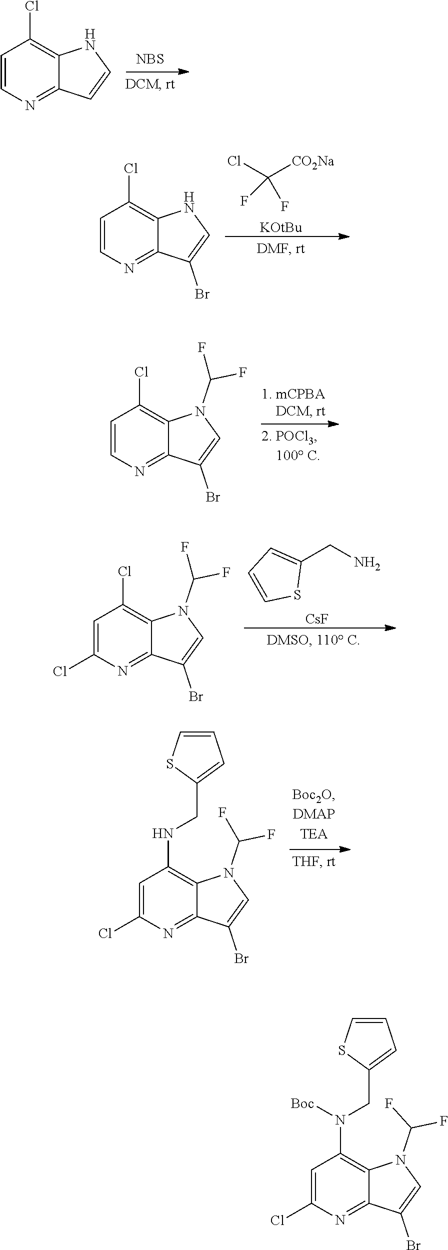

### Step 1: tert-Butyl (3-bromo-5-chloro-1-(difluoromethyl)-1H-pyrrolo[3,2-b]pyridin-7-yl)(thiophen-2-ylmethyl)carbamate

To a solution of 7-chloro-1H-pyrrolo[3,2-b]pyridine (500 mg, 2 mmol) in N,N-dimethylformamide (5 mL) was added 1-bromopyrrolidine-2,5-dione (700 mg, 4 mmol, 2 eq.). After stirring at room temperature for 4 h, the reaction mixture was diluted with water (50 mL) and extracted with EtOAc (40 mL×2). The combined organic layers were washed with brine (100 mL×2), dried over Na₂SO₄ and concentrated in vacuo to give crude product. The crude was triturated from DCM (20 mL) to give 3-bromo-7-chloro-1H-pyrrolo[3,2-b]pyridine (480 mg, 96%) as a yellow solid. MS m/z 233.1 [M+H]⁺.

### Step 2: 3-Bromo-7-chloro-1-(difluoromethyl)-1H-pyrrolo[3,2-b]pyridine

To a mixture of 3-bromo-7-chloro-1H-pyrrolo[3,2-b]pyridine (1 g, 4.3 mmol) in acetonitrile (15 mL) was added ethyl 2-bromo-2,2-difluoro-acetate (3.5 g, 17 mmol, 4 eq.) and potassium tert-butoxide (1.95 g, 17 mmol, 4 eq). After stirring at room temperature for 1 h, the reaction mixture was poured into water (100 mL) and extracted with EtOAc (70 mL×2). The combined organics were washed with brine (100 mL), dried over Na₂SO₄, filtered and concentrated in vacuo.

The crude residue was purified by silica gel column chromatography (EA/PE, 0-5% gradient) to give 3-bromo-7-chloro-1-(difluoromethyl)pyrrolo[3,2-b]pyridine (600 mg, 49%) as a yellow solid. MS m/z 282.9 [M+H]⁺.

### Step 3: 3-Bromo-5,7-dichloro-1-(difluoromethyl)-1H-pyrrolo[3,2-b]pyridine

To a solution of 3-bromo-7-chloro-1-(difluoromethyl)pyrrolo[3,2-b]pyridine (1.6 g, 5.7 mmol) in DCM (20 mL) was added 3-chloroperoxybenzoic acid (3.5 g, 17 mmol, 3 eq.) at 25° C. After stirring at room temperature for 16 h, the reaction mixture was filtered and the filtrate was concentrated in vacuo to afford crude 3-bromo-5,7-dichloro-1-(difluoromethyl)-1H-pyrrolo[3,2-b]pyridine as a white solid. To this crude material was added phosphoryl trichloride (20 mL) and the reaction mixture was heated at 90° C. for 1 h. The mixture was then cooled to room temperature and carefully poured into ice water (200 mL). The solution was extracted with EtOAc (2×200 mL). The combined organics were washed with saturated aqueous NaHCO₃ (3×150 mL), brine (150 mL), dried over Na₂SO₄, filtered, and concentrated to afford 3-bromo-5,7-dichloro-1-(difluoromethyl)-1H-pyrrolo[3,2-b]pyridine (660 mg, 37%) as a yellow solid. MS m/z 316.9 [M+H]⁺. ¹H NMR (400 MHz, DMSO-d₆) δ: 8.52 (s, 1H), 8.23 (t, J=59.8 Hz, 1H), 7.80 (s, 1H).

### Step 4: 3-Bromo-5-chloro-1-(difluoromethyl)-N-(thiophen-2-ylmethyl)-1H-pyrrolo[3,2-b]pyridin-7-amine

To a solution of 3-bromo-5,7-dichloro-1-(difluoromethyl)pyrrolo[3,2-b]pyridine (840 mg, 2.7 mmol) in DMSO (10 mL) was added 2-thienylmethanamine (330 mg, 3 mmol, 1.1 eq.) and cesium fluoride (808 mg, 5 mmol, 1.8 eq.) at room temperature. After stirring at 110° C. for 16 h, the reaction mixture was cooled to room temperature and poured into water (100 mL). The aqueous solution was extracted with EtOAc (2×100 mL) and the combined organics were washed with brine (2×100 mL), dried over Na₂SO₄, filtered, and concentrated in vacuo. The crude reside was purified by silica gel column chromatography (EA/Hex 1:1) to afford 3-bromo-5-chloro-1-(difluoromethyl)-N-(thiophen-2-ylmethyl)-1H-pyrrolo[3,2-b]pyridin-7-amine (590 mg, 51%) as a yellow solid. MS m/z 393.9 [M+H]⁺.

### Step 5: tert-Butyl (3-bromo-5-chloro-1-(difluoromethyl)-1H-pyrrolo[3,2-b]pyridin-7-yl)(thiophen-2-ylmethyl)carbamate

To a solution of 3-bromo-5-chloro-1-(difluoromethyl)-N-(2-thienylmethyl)pyrrolo[3,2-b]pyridin-7-amine (1.5 g, 3.8 mmol) in THF (20 mL) was added di-tert-butyl dicarbonate (1.74 g, 8 mmol, 2 eq.), triethylamine (0.8 g, 8 mmol, 2 eq.) and DMAP (0.05 g, 0.4 mmol, 0.1 eq.) at room temperature. After stirring at room temperature for 1 h, the reaction mixture was concentrated and the crude reside was purified by silica gel column chromatography (EA/PE 0-10% gradient) to afford: tert-butyl (3-bromo-5-chloro-1-(difluoromethyl)-1H-pyrrolo[3,2-b]pyridin-7-yl)(thiophen-2-ylmethyl)carbamate (2 g, 100%) as a yellow solid. MS m/z 494.0 [M+H]⁺. ¹H NMR (400 MHz, CHLOROFORM-d) δ: 7.71 (s, 1H), 7.27 (dd, J=5.2, 1.0 Hz, 1H), 7.18, (d, J=60.5 Hz, 1H), 6.97-6.90 (m, 2H), 4.97 (d, J=7.7 Hz, 2H), 1.41, (s, 9H).

### Intermediate 10

### tert-Butyl (3,5-dichloro-1-(difluoromethyl)-1H-pyrrolo[3,2-b]pyridin-7-yl)(thiophen-2-ylmethyl)carbamate

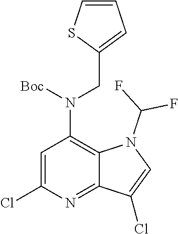 [SMILES: CC(C)(C)OC(=O)N(Cc1cccs1)c1cc(Cl)nc2c(Cl)cn(C(F)F)c12]

tert-Butyl (3,5-dichloro-1-(difluoromethyl)-1H-pyrrolo[3,2-b]pyridin-7-yl)(thiophen-2-ylmethyl)carbamate was prepared according to the procedure outlined in Intermediate 9, steps 1-5, by substituting the appropriate starting materials, reagents, and reaction conditions. MS m/z 448.1 [M+H]⁺. ¹H NMR (400 MHz, DMSO-d₆) δ: 8.44 (s, 1H), 7.80-7.40 (m, 2H), 7.07 (s, 1H), 6.96 (dt, J=8.4, 2.8 Hz, 2H), 5.17 (d, J=15.5 Hz, 1H), 4.86 (d, J=15.5 Hz, 1H), 1.36 (s, 9H).

### Intermediate 11

### tert-Butyl (5-chloro-1-(difluoromethyl)-3-iodo-1H-pyrrolo[3,2-b]pyridin-7-yl)(thiophen-2-ylmethyl)carbamate

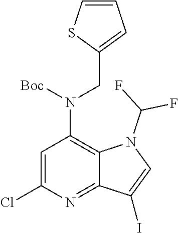 [SMILES: CC(C)(C)OC(=O)N(Cc1cccs1)c1cc(Cl)nc2c(I)cn(C(F)F)c12]

tert-Butyl (3,5-dichloro-1-(difluoromethyl)-1H-pyrrolo[3,2-b]pyridin-7-yl)(thiophen-2-ylmethyl)carbamate was prepared according to the procedure outlined in Intermediate 9, steps 1-5, by substituting the appropriate starting materials, reagents, and reaction conditions. MS m/z 540.1 [M+H]⁺.

### Intermediate 12

### tert-Butyl (5-chloro-1-(difluoromethyl)-3-iodo-1H-pyrrolo[3,2-b]pyridin-7-yl)(furan-2-ylmethyl)carbamate

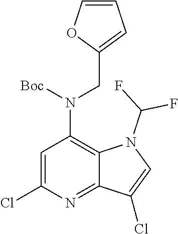 [SMILES: CC(C)(C)OC(=O)N(Cc1ccco1)c1cc(Cl)nc2c(Cl)cn(C(F)F)c12]

tert-Butyl (3,5-dichloro-1-(difluoromethyl)-1H-pyrrolo[3,2-b]pyridin-7-yl)(thiophen-2-ylmethyl)carbamate was prepared according to the procedure outlined in Intermediate 9, steps 1-5, by substituting the appropriate starting materials, reagents, and reaction conditions. MS m/z 432.0 [M+H]⁺.

### Intermediate 13

### tert-Butyl (3-bromo-5-chloro-1-(difluoromethyl)-1H-pyrrolo[3,2-b]pyridin-7-yl)(furan-2-ylmethyl)carbamate

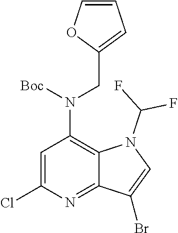 [SMILES: CC(C)(C)OC(=O)N(Cc1ccco1)c1cc(Cl)nc2c(Br)cn(C(F)F)c12]

tert-Butyl (3,5-dichloro-1-(difluoromethyl)-1H-pyrrolo[3,2-b]pyridin-7-yl)(thiophen-2-ylmethyl)carbamate was prepared according to the procedure outlined in Intermediate 9, steps 1-5, by substituting the appropriate starting materials, reagents, and reaction conditions. MS m/z 478.3 [M+H]⁺. ¹H NMR (400 MHz, CHLOROFORM-d) δ: 7.71 (s, 1H), 7.30-7.12 (m, 1H), 7.04 (s, 1H), 6.25 (s, 1H), 6.17 (d, J=3.0 Hz, 1H), 5.04 (d, J=15.9 Hz, 1H), 4.67 (d, J=15.5 Hz, 1H), 1.37 (s, 9H).

### Intermediate 14

### tert-Butyl (3,5-dichloro-1-(difluoromethyl)-1H-pyrrolo[3,2-b]pyridin-7-yl)(thiophen-2-ylmethyl)carbamate

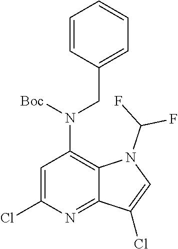 [SMILES: CC(C)(C)OC(=O)N(Cc1ccccc1)c1cc(Cl)nc2c(Cl)cn(C(F)F)c12]

tert-Butyl (3,5-dichloro-1-(difluoromethyl)-1H-pyrrolo[3,2-b]pyridin-7-yl)(thiophen-2-ylmethyl)carbamate was prepared according to the procedure outlined in Intermediate 9, steps 1-5, by substituting the appropriate starting materials, reagents, and reaction conditions. MS m/z 442.2 [M+H]⁺.

### Intermediate 15

### tert-Butyl (3,5-dichloro-1-(difluoromethyl)-1H-pyrrolo[3,2-b]pyridin-7-yl)(thiophen-2-ylmethyl)carbamate

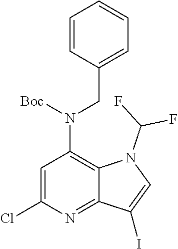 [SMILES: CC(C)(C)OC(=O)N(Cc1ccccc1)c1cc(Cl)nc2c(I)cn(C(F)F)c12]

tert-Butyl (3,5-dichloro-1-(difluoromethyl)-1H-pyrrolo[3,2-b]pyridin-7-yl)(thiophen-2-ylmethyl)carbamate was prepared according to the procedure outlined in Intermediate 9, steps 1-5, by substituting the appropriate starting materials, reagents, and reaction conditions. MS m/z 534.3 [M+H]⁺.

### Intermediate 16

### tert-Butyl (5-chloro-3-(trifluoromethyl)thieno[3,2-b]pyridin-7-yl)(thiophen-2-ylmethyl)carbamate

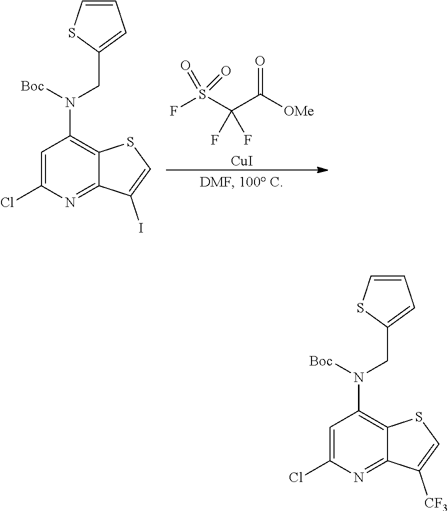

A mixture of tert-butyl (5-chloro-3-iodothieno[3,2-b]pyridin-7-yl)(thiophen-2-ylmethyl)carbamate (200 mg, 0.4 mmol, 1.0 eq.), methyl 2,2-difluoro-2-fluorosulfonyl-acetate (0.15 mL, 1.2 mmol, 3 eq.), CuI (75 mg, 0.4 mmol, 1.0 eq.) in DMF (1 mL) was heated at 100° C. for 5 h. The mixture was concentrated in vacuo and the crude residue was diluted with EtOAc and the solids were removed by filtration. The filtrate was washed with water (50 mL×2), brine (100 mL), dried over Na₂SO₄, filtered, and concentrated in vacuo. The crude residue was purified by silica gel column chromatography (EA/Hex 0-50% gradient) to afford tert-butyl (5-chloro-3-(trifluoromethyl)thieno[3,2-b]pyridin-7-yl)(thiophen-2-ylmethyl)carbamate (177 mg, 56%) as an off white solid. MS m/z 448.9 [M+H]⁺. ¹H NMR (400 MHz, CHLOROFORM-d) δ: ppm 8.19 (s, 1H) 7.26 (d, J=5.00 Hz, 1H) 7.19 (s, 1H) 6.90 (t, J=4.25 Hz, 1H) 6.83 (d, J=3.00 Hz, 1H) 5.08 (s, 2H) 1.49 (s, 9H).

### Intermediate 17

### tert-butyl (5-chloro-3-(difluoromethyl)thieno[3,2-b]pyridin-7-yl)(thiophen-2-ylmethyl)carbamate

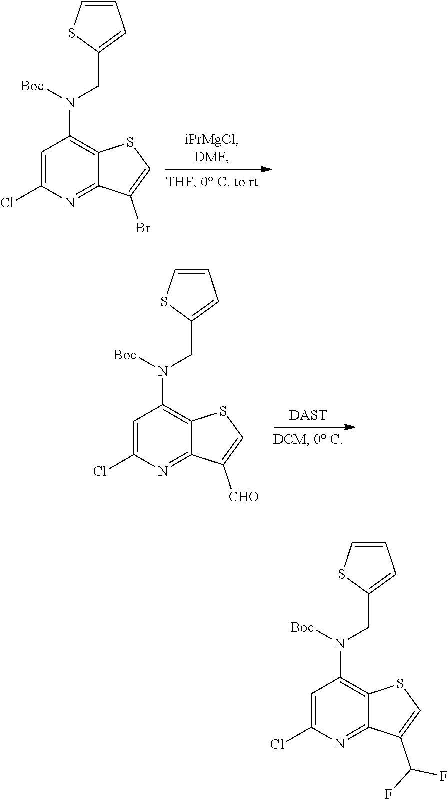

### Step 1: tert-Butyl (5-chloro-3-formylthieno[3,2-b]pyridin-7-yl)(thiophen-2-ylmethyl)carbamate

To a solution of tert-butyl (3-bromo-5-chlorothieno[3,2-b]pyridin-7-yl)(thiophen-2-ylmethyl)carbamate (1.0 g, 2.2 mmol) (prepared according to the procedure described in WO2020/17430) in THF (18 mL), cooled to 0° C., was added 2M isopropylmagnesium chloride (1.5 mL, 3 mmol, 1.4 eq.). After stirring at room temperature for 1 hr, DMF (1.7 mL, 20 mmol, 10 eq.) was added and the mixture was stirred for an additional 1 hr. The reaction mixture was quenched with saturated aqueous NH₄Cl (50 mL) and diluted with water (100 mL). The aqueous phase was extracted with EtOAc (2×100 mL) and the combined organics were dried over MgSO₄, filtered, and concentrated in vacuo. The crude residue was purified by silica gel column chromatography (EA/Hex 0-50% gradient) to afford tert-butyl (5-chloro-3-formylthieno[3,2-b]pyridin-7-yl)(thiophen-2-ylmethyl)carbamate (889 mg, 70%) as a yellow oil. MS m/z 430.7 [M+Na]⁺. ¹H NMR (400 MHz, CHLOROFORM-d) δ: ppm 10.47 (s, 1H) 8.62 (s, 1H) 7.22-7.29 (m, 2H) 6.80-6.95 (m, 2H) 5.10 (s, 2H) 1.49 (s, 9H).

### Step 2: tert-butyl (5-chloro-3-(difluoromethyl)thieno[3,2-b]pyridin-7-yl)(thiophen-2-ylmethyl)carbamate

To a solution of afford tert-butyl (5-chloro-3-formylthieno[3,2-b]pyridin-7-yl)(thiophen-2-ylmethyl)carbamate (120 mg, 0.3 mmol) in DCM (0.5 mL), cooled to 0° C., was added DAST (0.08 mL, 0.6 mmol, 2 eq.). After stirring at 0° C. for 1 h, the reaction mixture was allowed to warm to room temperature and stirred overnight. The reaction mixture was then cooled to 0° C. and quenched with aqueous NaHCO₃ (2M, 5 mL). After neutralization, the mixture was extracted with EtOAc (3×10 mL). The combined organics were washed with brine (10 mL), dried over Na₂SO₄, filtered, and concentrated in vacuo. The crude residue was purified by silica gel column chromatography (EA/Hex 0-40% gradient) to afford tert-butyl (5-chloro-3-(difluoromethyl)thieno[3,2-b]pyridin-7-yl)(thiophen-2-ylmethyl)carbamate (100 mg, 79%) as a yellow oil. MS m/z 431.0 [M+H]⁺. ¹H NMR (400 MHz, CHLOROFORM-d) δ: ppm 8.12 (s, 1H) 6.98-7.28 (m, 3H) 6.80-6.93 (m, 2H) 5.08 (s, 2H) 1.49 (s, 9H).

### Intermediate 18

### tert-Butyl (5-chloro-3-(fluoromethyl)thieno[3,2-b]pyridin-7-yl)(thiophen-2-ylmethyl)carbamate

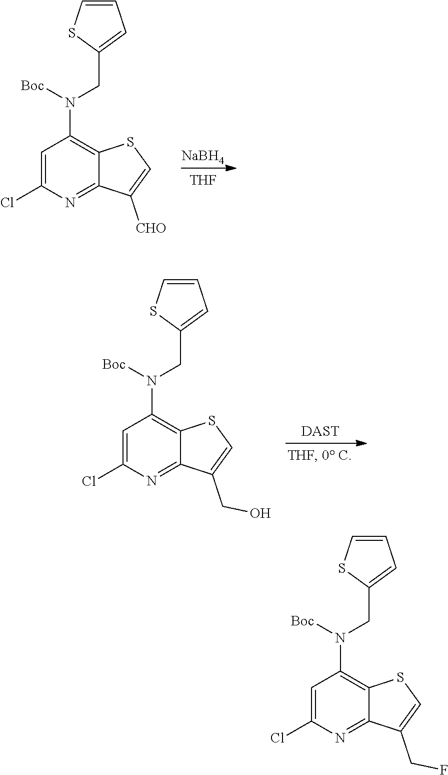

### Step 1: tert-Butyl (5-chloro-3-(hydroxymethyl)thieno[3,2-b]pyridin-7-yl)(thiophen-2-ylmethyl)carbamate

To a solution of tert-butyl (5-chloro-3-formylthieno[3,2-b]pyridin-7-yl)(thiophen-2-ylmethyl)carbamate (300 mg, 0.7 mmol) (prepared according to step 1 of Intermediate 17) in THF (2.7 mL), cooled to 0° C., was added NaBH₄ (17 mg, 0.4 mmol, 0.6 eq.). After stirring at room temperature for 1 h, the reaction mixture was diluted with water and extracted with EtOAc (2×50 mL). The combined organics were dried over Na₂SO₄, filtered, and concentrated in vacuo. The crude residue was purified by silica gel column chromatography (EA/Hex 0-50% gradient) to afford tert-butyl (5-chloro-3-(hydroxymethyl)thieno[3,2-b]pyridin-7-yl)(thiophen-2-ylmethyl)carbamate (220 mg, 73%) as a yellow oil. MS m/z 411.0 [M+H]⁺. ¹H NMR (400 MHz, CHLOROFORM-d) δ: ppm 7.67 (s, 1H) 7.25 (d, J=5.13 Hz, 1H) 7.10 (s, 1H) 6.79-6.92 (m, 2H) 5.08 (s, 2H) 5.00 (s, 2H) 1.48 (s, 9H).

### Step 2: tert-Butyl (5-chloro-3-(fluoromethyl)thieno[3,2-b]pyridin-7-yl)(thiophen-2-ylmethyl)carbamate

To a solution of tert-butyl (5-chloro-3-(hydroxymethyl)thieno[3,2-b]pyridin-7-yl)(thiophen-2-ylmethyl)carbamate (630 mg, 1.5 mmol) in DCM (3 mL), cooled to 0° C., was added DAST (0.4 mL, 3 mmol, 2 eq.). After stirring at room temperature for 1 h, the reaction was quenched with saturated aqueous NaHCO₃ (5 mL) and diluted with water (50 mL). The aqueous phase was extracted with EtOAc (2×100 mL) and the combined organics were dried over Na₂SO₄, filtered, and concentrated in vacuo. The crude residue was purified by silica gel column chromatography (EA/Hex 0-50% gradient) to afford tert-butyl (5-chloro-3-(fluoromethyl)thieno[3,2-b]pyridin-7-yl)(thiophen-2-ylmethyl)carbamate (150 mg, 24%) as a yellow oil. MS m/z 413.0 [M+H]⁺. ¹H NMR (400 MHz, CHLOROFORM-d) δ: ppm 7.76 (d, J=1.88 Hz, 1H) 7.15 (d, J=5.00 Hz, 1H) 7.02 (s, 1H) 6.68-6.86 (m, 2H) 5.54-5.73 (m, 2H) 4.99 (s, 2H) 1.39 (s, 9H).

### Intermediate 19

### tert-Butyl (5-chloro-3-((difluoromethoxy)methyl)thieno[3,2-b]pyridin-7-yl)(thiophen-2-ylmethyl)carbamate

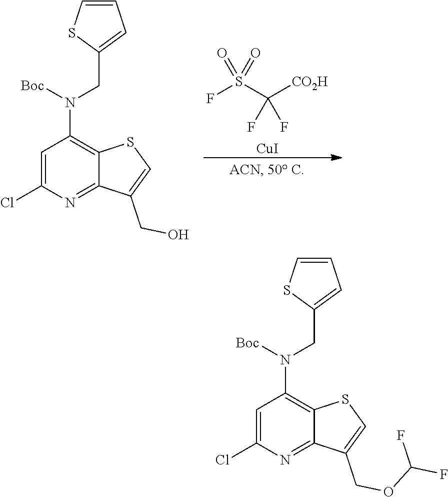

### Step 1: tert-Butyl (5-chloro-3-((difluoromethoxy)methyl)thieno[3,2-b]pyridin-7-yl)(thiophen-2-ylmethyl)carbamate

A mixture of tert-butyl (5-chloro-3-(hydroxymethyl)thieno[3,2-b]pyridin-7-yl)(thiophen-2-ylmethyl)carbamate (200 mg, 0.5 mmol) (prepared according to the procedure described in Intermediate 18) and CuI (19 mg, 0.01 mmol, 0.2 eq.) in acetonitrile (1 mL) was stirred at 50° C. To this mixture was added dropwise 2,2-difluoro-2-(fluorosulfonyl)acetic acid (0.1 mL, 1 mmol, 2 eq.). After stirring at 50° C. for 1 h, the reaction mixture was allowed to cool to room temperature and concentrated in vacuo. The crude residue was purified by silica gel column chromatography to afford tert-butyl (5-chloro-3-((difluoromethoxy)methyl)thieno[3,2-b]pyridin-7-yl)(thiophen-2-ylmethyl)carbamate (100 mg, 45%) as a yellow oil. MS m/z 461.0 [M+H]⁺. ¹H NMR (400 MHz, CHLOROFORM-d) δ: ppm 7.77-7.82 (m, 1H) 7.25 (d, J=5.00 Hz, 1H) 7.11 (s, 1H) 6.90 (t, J=4.13 Hz, 1H) 6.82 (br d, J=2.63 Hz, 1H) 6.23-6.65 (m, 1H) 5.26 (s, 2H) 5.08 (s, 2H) 1.48 (s, 9H).

### Intermediate 20

### tert-Butyl (E)-(3-bromo-5-chloro-2-(2-nitrovinyl)thieno[3,2-b]pyridin-7-yl)(thiophen-2-ylmethyl)carbamate

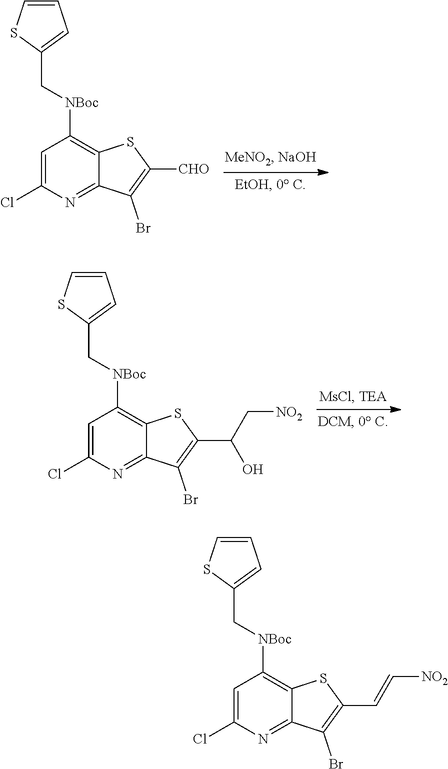 [SMILES: CC(C)(C)OC(=O)N(Cc1cccs1)c1cc(Cl)nc2c(Br)c(C=O)sc12 ; CC(C)(C)OC(=O)N(Cc1cccs1)c1cc(Cl)nc2c(Br)c(C(O)C[N+](=O)[O-])sc12 ; CC(C)(C)OC(=O)N(Cc1cccs1)c1cc(Cl)nc2c(Br)c(/C=C/[N+](=O)[O-])sc12]

### Step 1: tert-Butyl (3-bromo-5-chloro-2-(1-hydroxy-2-nitroethyl)thieno[3,2-b]pyridin-7-yl)(thiophen-2-ylmethyl)carbamate

To a stirred solution of tert-butyl (3-bromo-5-chloro-2-formylthieno[3,2-b]pyridin-7-yl)(thiophen-2-ylmethyl)carbamate (3 g, 6.2 mmol) and nitromethane (1.2 mL, 22.4 mmol, 3.5 eq.) in EtOH (50 mL), cooled to 0° C.) was added 50% aq. NaOH (7.2 mL). After stirring for 1 h at 0° C., the reaction mixture was slowly quenched with 2M HCl (20 mL) and then diluted with water (100 mL). The aqueous phase was extracted with EtOAc (2×100 mL) and the combined organics were dried over Na₂SO₄, filtered, and concentrated in vacuo. The crude residue was by silica gel column chromatography (EA/Hex 0-50% gradient) to afford tert-butyl (3-bromo-5-chloro-2-(1-hydroxy-2-nitroethyl)thieno[3,2-b]pyridin-7-yl)(thiophen-2-ylmethyl)carbamate (3.4 g, 77%) as a yellow oil. MS m/z 549.6 [M+H]⁺.

### Step 2: tert-Butyl (E)-(3-bromo-5-chloro-2-(2-nitrovinyl)thieno[3,2-b]pyridin-7-yl)(thiophen-2-ylmethyl)carbamate

To a solution of afford tert-butyl (3-bromo-5-chloro-2-(1-hydroxy-2-nitroethyl)thieno[3,2-b]pyridin-7-yl)(thiophen-2-ylmethyl)carbamate (310 mg, 0.2 mmol) in DCM (2 mL), cooled to 0° C., was added methanesulfonyl chloride (0.05 mL, 0.22 mmol, 1.1 eq.) and TEA (0.2 mL, 5 mmol, 2.2 eq.). After stirring for 2 h, the reaction mixture was diluted with water (50 mL) and extracted with DCM (2×50 mL). The combined organics were washed with brine, dried over Na₂SO₄, filtered, and concentrated in vacuo. The crude residue was purified by silica gel column chromatography (EA/Hex 0-50% gradient) to afford tert-butyl (E)-(3-bromo-5-chloro-2-(2-nitrovinyl)thieno[3,2-b]pyridin-7-yl)(thiophen-2-ylmethyl)carbamate (300 mg, 93%) as a yellow oil. MS m/z 531.9 [M+H]⁺. ¹H NMR (CHLOROFORM-d) δ: 8.41 (d, J=13.5 Hz, 1H), 7.64 (d, J=13.5 Hz, 1H), 7.24-7.28 (m, 2H), 6.92 (dd, J=5.0, 3.5 Hz, 1H), 6.84 (d, J=3.4 Hz, 1H), 5.07 (s, 2H), 1.52 (s, 9H).

### Example 1

### Preparation of Compound 72, Compound 73, Compound 74, and Compound 75

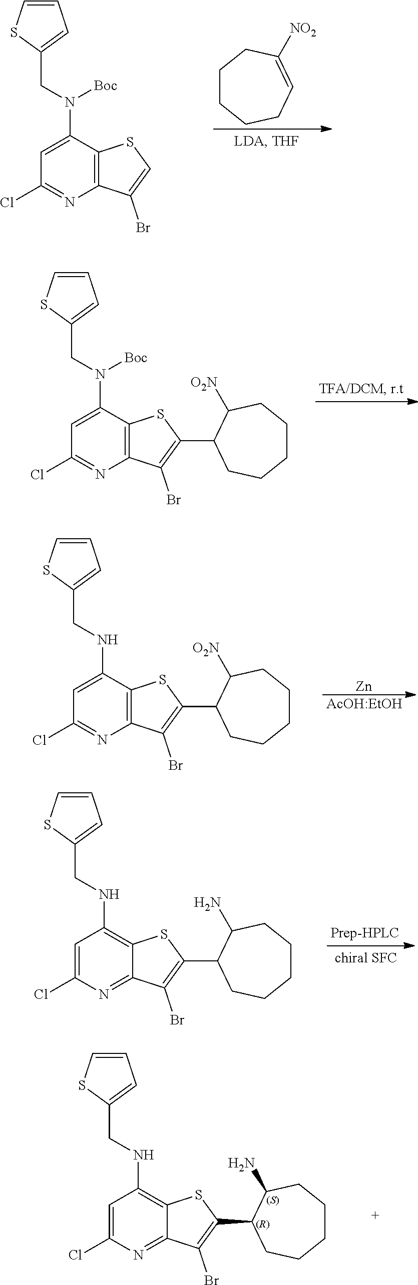

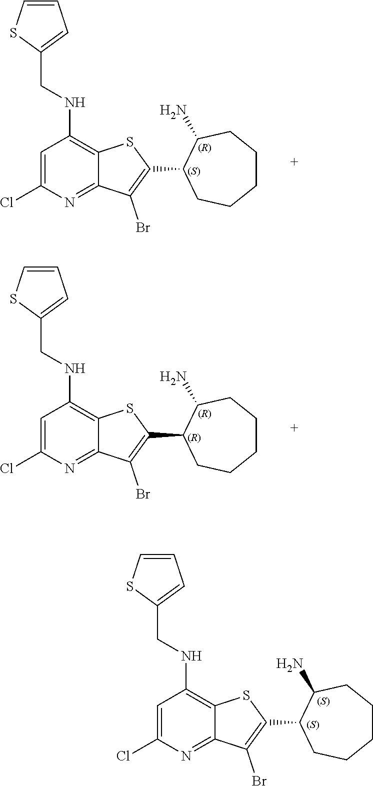

### Step 1: tert-Butyl (3-bromo-5-chloro-2-(2-nitrocycloheptyl)thieno[3,2-b]pyridin-7-yl)(thiophen-2-ylmethyl)carbamate

To a solution of tert-butyl (3-bromo-5-chlorothieno[3,2-b]pyridin-7-yl)(thiophen-2-ylmethyl)carbamate (1 g, 2.17 mmol) in THF (10 mL) was added lithium diisopropylamide (2M) in THF/heptane/ethylbenzene (1.63 mL, 3.26 mmol, 1.5 eq) at −78° C. The mixture was stirred at −78° C. for 30 min. Then 1-nitrocyclohept-1-ene (397.8 mg, 2.82 mmol) was added, the mixture was stirred at −78° C. for 3 hours. TLC showed the starting material was remained and new spot was observed. The reaction mixture was diluted by adding NH₄Cl (sat. aq.) (125 mL). The organic layer was extracted with EtOAc (125 ml×3). The combined organic layers were washed with brine, dried over Na₂SO₄, filtered, and then concentrated in vacuo. The crude residue was purified by flash column chromatography on silica gel eluting with ethyl acetates in petroleum ether (0 to 50% gradient) to give tert-butyl (3-bromo-5-chloro-2-(2-nitrocycloheptyl)thieno[3,2-b]pyridin-7-yl)(thiophen-2-ylmethyl)carbamate (260 mg, 0.43 mmol) as white solid.

### Step 2: 3-Bromo-5-chloro-2-(2-nitrocycloheptyl)-N-(thiophen-2-ylmethyl)thieno[3,2-b]pyridin-7-amine

To a solution of tert-butyl (3-bromo-5-chloro-2-(2-nitrocycloheptyl)thieno[3,2-b]pyridin-7-yl)(thiophen-2-ylmethyl)carbamate (50 mg, 0.08 mmol) in CH₂Cl₂ (8 ml) was added TFA (4 mL) at 0° C. After stirring for 30 minutes at 25° C., the mixture was concentrated in vacuo to give crude 3-bromo-5-chloro-2-(2-nitrocycloheptyl)-N-(2-thienylmethyl)thieno[3,2-b]pyridin-7-amine (40 mg, 0.08 mmol 96% Yield) as a white solid which was used in the next step without further purification. MS m/z 501.8 [M+H]⁺

### Step 3: 2-(2-Aminocycloheptyl)-3-bromo-5-chloro-N-(thiophen-2-ylmethyl)thieno[3,2-b]pyridin-7-amine

To a solution of 3-bromo-5-chloro-2-(2-nitrocycloheptyl)-N-(2-thienylmethyl)thieno[3,2-b]pyridin-7-amine (150 mg, 0.3 mmol) and zinc (98 mg, 1.5 mmol) in ethanol (8 mL) was added TFA (2 mL, 26.5 mmol) at 0° C. After stirring the reaction for 30 minutes at 70° C., the mixture was concentrated and the crude oil was purified by PreP-HPLC to give trans-2-(2-aminocycloheptyl)-3-bromo-5-chloro-N-(2-thienylmethyl)thieno[3,2-b]pyridin-7-amine (25 mg, 0.05 mmol, 18% Yield) as an off-white solid and cis-2-(2-aminocycloheptyl)-3-bromo-5-chloro-N-(2-thienylmethyl)thieno[3,2-b]pyridin-7-amine (60 mg, 0.1 mmol, 43% Yield) as an off-white solid. MS m/z 471.8 [M+H]⁺

### Step 4: 2-((1S,2R)-2-Aminocycloheptyl)-3-bromo-5-chloro-N-(thiophen-2-ylmethyl)thieno[3,2-b]pyridin-7-amine, 2-((1R,2S)-2-aminocycloheptyl)-3-bromo-5-chloro-N-(thiophen-2-ylmethyl)thieno[3,2-b]pyridin-7-amine, 2-((1S,2S)-2-aminocycloheptyl)-3-bromo-5-chloro-N-(thiophen-2-ylmethyl)thieno[3,2-b]pyridin-7-amine, and 2-((1R,2R)-2-aminocycloheptyl)-3-bromo-5-chloro-N-(thiophen-2-ylmethyl)thieno[3,2-b]pyridin-7-amine

Cis-2-(2-aminocycloheptyl)-3-bromo-5-chloro-N-(2-thienylmethyl)thieno[3,2-b]pyridin-7-amine (60 mg, 0.13 mmol) was purified by Prep-SFC (Dr.maish Reprosil Chiral-AM, Supercritical CO₂/MeOH (+0.1% 7.0 mol/l NH₃ in MeOH)/MeOH) to give 2-[(1R,2R)-2-aminocycloheptyl]-3-bromo-5-chloro-N-(2-thienylmethyl)thieno[3,2-b]pyridin-7-amine (Compound 72, 11 mg, 0.02 mmol, 18% Yield) and 2-[(1S,2S)-2-aminocycloheptyl]-3-bromo-5-chloro-N-(2-thienylmethyl)thieno[3,2-b]pyridin-7-amine (Compound 73, 16 mg, 0.03 mmol, 27% Yield) as an off-white solid. 2-[(1R,2S)-2-aminocycloheptyl]-3-bromo-5-chloro-N-(2-thienylmethyl)thieno[3,2-b]pyridin-7-amine (Compound 74, 2.8 mg, 0.006 mmol, 14% Yield) and P2-[(1S,2R)-2-aminocycloheptyl]-3-bromo-5-chloro-N-(2-thienylmethyl)thieno[3,2-b]pyridin-7-amine (Compound 75, 2.8 mg, 0.006 mmol, 14% Yield) as an off-white solid.

Compound 72: MS m/z 472.0 [M+H]⁺; ¹H NMR (DMSO-d₆) δ ppm 7.97 (t, J=5.8 Hz, 1H), 7.40 (d, J=5.0 Hz, 1H), 7.10 (d, J=2.6 Hz, 1H), 7.00-6.98 (m, 1H), 6.60 (s, 1H), 4.70 (d, J=5.8 Hz, 2H), 3.50 (d, J=12 Hz, 1H), 3.31-3.27 (m, 1H), 1.95-1.77 (m, 11H), 1.23 (s, 1H).

Compound 73: MS m/z 472.0 [M+H]⁺; ¹H NMR (DMSO-d₆) δ ppm 7.99 (t, J=5.8 Hz, 1H), 7.40 (d, J=5.2 Hz, 1H), 7.10 (d, J=2.6 Hz, 1H), 7.00-6.98 (m, 1H), 6.58 (s, 1H), 4.71 (d, J=6.0 Hz, 2H), 3.55 (d, J=12 Hz, 1H), 3.37-3.27 (m, 1H), 2.08-1.43 (m, 11H), 1.25 (s, 1H).

Compound 74: MS m/z 471.9 [M+H]⁺; ¹H NMR (DMSO-d₆) δ ppm 7.96 (s, 1H), 7.40 (d, J=4.0 Hz, 1H), 7.10 (s, 1H), 7.05-6.92 (m, 1H), 6.57 (s, 1H), 4.72 (d, J=5.6 Hz, 2H), 3.20-3.13 (m, 2H), 1.99-1.39 (m, 12H).

Compound 75: MS m/z 471.9 [M+H]⁺; ¹H NMR (DMSO-d₆) δ ppm 7.95 (t, J=5.8 Hz, 1H), 7.40 (d, J=5.0 Hz, 1H), 7.11 (d, J=2.6 Hz, 1H), 7.00-6.98 (m, 1H), 6.60 (s, 1H), 4.72 (d, J=5.8 Hz, 2H), 3.25-3.13 (m, 2H), 1.95-1.45 (m, 12H).

The compounds below were prepared according to the procedure of Example 1 by substituting the appropriate starting materials, reagents, reaction conditions, and followed by chiral SFC resolution if needed.

| Compound | Spectral Data |
|---|---|
| 1 | MS m/z 398.1 [M + H]⁺; ¹HNMR (400 MHz, DMSO-d₆) δ: 7.97 (s, 1H), |
| | 7.42-7.34 (m, 1H), 7.11 (s, 1H), 6.99 (d, J = 3.4 Hz, 1H), 6.61 (d, J = |
| | 4.5 Hz, 1H), 4.73 (s, 2H), 2.11-1.57 (m, 7H), 1.49 (m, 1H), 1.25 (m, |
| | 2H) |
| 2 | MS m/z 398.1 [M + H]⁺; ¹HNMR(400 MHz, DMSO-d₆) δ: 7.97 (s, 1H), |
| | 7.40 (d, J = 5.0 Hz, 1H), 7.11 (s, 1H), 6.98 (d, J = 3.4 Hz, 1H), 6.62 (d, |
| | J = 4.6 Hz, 1H), 4.72 (s, 2H), 2.11-1.57 (m, 7H), 1.49 (m, 1H), 1.25 |
| | (m, 2H) |
| 3 | MS m/z 398.1 [M + H]⁺; 1HNMR(400 MHz, DMSO-d₆) δ: 7.98 (t, J = |
| | 5.9 Hz, 1H), 7.40 (d, J = 4.9 Hz, 1H), 7.10 (d, J = 3.1 Hz, 1H), 7.04- |
| | 6.93 (m, 1H), 6.58 (s, 1H), 4.71 (d, J = 5.9 Hz, 2H), 2.16-1.84 (m, |
| | 6H), 1.77-1.40 (m, 4H) |
| 4 | MS m/z 398.1 [M + H]⁺; ¹HNMR(400 MHz, DMSO-d₆) δ: 7.98 (t, J = |
| | 5.8 Hz, 1H), 7.40 (d, J = 5.0 Hz, 1H), 7.10 (s, 1H), 7.00-6.96 (m, 1H), |
| | 6.58 (s, 1H), 4.71 (d, J = 5.9 Hz, 2H), 2.16-1.84 (m, 6H), 1.77-1.40 |
| | (m, 4H) |
| 13 | MS m/z 412.1 [M + H]⁺; ¹HNMR(400 MHz, METHANOL-d₄) δ: 7.29 |
| | (dd, J = 5.1, 0.9 Hz, 1H), 7.08 (d, J = 2.9 Hz, 1H), 6.97 (dd, J = 5.0, 3.5 |
| | Hz, 1H), 6.56 (s, 1H), 4.75 (s, 2H), 3.60 (dt, J = 11.7, 3.6 Hz, 1H), 3.45 |
| | (d, J = 3.5 Hz, 1H), 2.03 (dd, J = 12.6, 3.5 Hz, 1H), 1.93-1.69 (m, 5H), |
| | 1.61-1.48 (m, 2H). (3 exchangeable protons are not seen) |
| 14 | MS m/z 412.1 [M + H]⁺; ¹HNMR(400 MHz, METHANOL-d₄) δ: 7.29 |
| | (dd, J = 5.1, 0.9 Hz, 1H), 7.08 (d, J = 3.4 Hz, 1H), 6.97 (dd, J = 5.0, 3.5 |
| | Hz, 1H), 6.56 (s, 1H), 4.75 (s, 2H), 3.63 (d, J = 11.4 Hz, 1H), 3.49 (d, |
| | J = 3.6 Hz, 1H), 2.08-1.97 (m, 1H), 1.93-1.69 (m, 5H), 1.62-1.50 (m, |
| | 2H). (3 exchangeable protons are not seen) |
| 15 | MS m/z 459.9 [M + H]⁺; ¹H NMR (400 MHz, DMSO-d₆) δ: 8.03 (s, 1H), |
| | 7.41-7.39 (m, 1H), 7.10 (s, 1H), 7.00-6.98 (m, 1H), 6.63 (s, 1H), 5.07 |
| | (s, 1H), 4.75-4.73 (m, 2H), 4.05-4.00 (m, 1H), 3.75-3.65(m, 1H), |
| | 3.59 (s, 1H), 3.37 (d, J = 12 Hz, 1H), 3.27 (s, J = 12 Hz, 1H), 2.15- |
| | 1.95 (m, 3H), 1.47-1.44 (m, 1H). |
| 16 | MS m/z 414.0 [M + H]⁺; ¹HNMR(400 MHz, METHANOL-d₄) δ: 7.29 |
| | (dd, J = 5.1, 1.1 Hz, 1H), 7.09-7.06 (m, 1H), 6.97 (dd, J = 5.1, 3.5 Hz, |
| | 1H), 6.59 (s, 1H), 4.76 (s, 2H), 4.66 (d, J = 9.4 Hz, 1H), 4.08-4.01(m, |
| | 1H), 3.59 (t, J = 11.6, 3.0 Hz, 1H), 2.83 (ddd, J = 11.3, 9.4, 4.1 Hz, 1H), |
| | 2.20-2.11 (m, 1H), 1.90-1.73 (m, 2H), 1.56 (ddd, J = 24.2, 12.7, 4.5 |
| | Hz, 1H). (3 exchangeable protons are not seen) |
| 17 | MS m/z 414.0 [M + H]⁺; ¹HNMR(400 MHz, METHANOL-d₄) δ: 7.29 |
| | (dd, J = 5.1, 1.2 Hz, 1H), 7.07 (dd, J = 3.4, 0.9 Hz, 1H), 6.97 (dd, J = |
| | 5.1, 3.5 Hz, 1H), 6.55 (s, 1H), 5.06 (d, J = 1.8 Hz, 1H), 4.75 (s, 2H), |
| | 4.14 (dd, J = 10.5, 3.6 Hz, 1H), 3.73 (td, J = 11.7, 2.8 Hz, 1H), 3.46 (d, |
| | J = 1.8 Hz, 1H), 2.01 (ddd, J = 17.9, 11.7, 5.1 Hz, 3H), 1.48 (d, J = 8.1 |
| | Hz, 1H). (3 exchangeable protons are not seen) |
| 18 | MS m/z 414.1 [M + H]⁺; ¹HNMR(400 MHz, METHANOL-d₄) δ: 7.30 |
| | (dd, J = 5.1, 1.2 Hz, 1H), 7.08 (dd, J = 3.5, 1.1 Hz, 1H), 6.97 (dd, J = |
| | 5.1, 3.5 Hz, 1H), 6.60 (s, 1H), 4.76 (s, 2H), 4.67 (d, J = 9.4 Hz, 1H), |
| | 4.05 (dd, J = 11.5, 4.5 Hz, 1H), 3.59 (td, J = 11.6, 3.0 Hz, 1H), 2.84 |
| | (ddd, J = 11.3, 9.5, 4.1 Hz, 1H), 2.16 (d, J = 9.4 Hz, 1H), 1.81 (dt, J = |
| | 13.5, 9.7 Hz, 2H), 1.57 (ddd, J = 24.1, 12.8, 4.6 Hz, 1H). (3 |
| | exchangeable protons are not seen) |
| 19 | MS m/z 414.0 [M + H]⁺; ¹HNMR(400 MHz, METHANOL-d₄) δ: 7.29 |
| | (dd, J = 5.1, 1.2 Hz, 1H), 7.10-7.05 (m, 1H), 6.97 (dd, J = 5.1, 3.5 Hz, |
| | 1H), 6.55 (s, 1H), 5.06 (d, J = 1.8 Hz, 1H), 4.74 (s, 2H), 4.18-4.10 (m, |
| | 1H), 3.73 (td, J = 11.6, 2.8 Hz, 1H), 3.46 (d, J = 1.7 Hz, 1H), 2.08-1.93 |
| | (m, 3H), 1.48 (d, J = 8.0 Hz, 1H). (3 exchangeable protons are not seen) |
| 20 | MS m/z 459.9 [M + H]⁺; ¹H NMR (400 MHz, DMSO-d₆) δ: 7.97-7.95 |
| | (m, 1H), 7.41-7.39 (m, 1H), 7.10 (s, 1H), 7.00-6.97 (m, 1H), 6.59 (s, |
| | 1H), 4.92 (s, 1H), 4.75-4.71 (m, 2H), 4.03-3.99 (m, 1H), 3.68-3.63 |
| | (m, 1H), 3.32-3.27 (m, 1H), 2.08-1.65 (m, 5H), 1.38-1.35 m, 1H), |
| | 1.24 (s, 1H) |
| 26 | MS m/z 373.9, 376.0 [M + H]⁺; ¹H NMR (METHANOL-d₄) δ: 7.22-7.42 |
| | (m, 6H), 6.42-6.49 (m, 1H), 4.67-4.77 (m, 1H), 4.61 (s, 2H), 4.08-4.18 |
| | (m, 1H), 3.61-3.74 (m, 1H), 3.34-3.41 (m, 1H), 2.27-2.39 (m, 1H), 1.85- |
| | 1.99 (m, 2H), 1.71-1.85 (m, 1H). trifluoracetic acid salt, (3 |
| | exchangeable protons are not seen) |
| 27 | MS m/z 453.28 [M + H]⁺; ¹H NMR (METHANOL-d₄) δ: 7.30-7.40 (m, |
| | 4H), 7.23-7.30 (m, 1H), 7.22-7.31 (m, 1H), 6.48 (s, 1H), 4.95-5.04 (m, |
| | 1H), 4.55-4.65 (m, 2H), 4.11-4.21 (m, 1H), 3.63-3.75 (m, 1H), 3.35- |
| | 3.46 (m, 1H), 2.29-2.39 (m, 1H), 1.80-2.00 (m, 2H). trifluoracetic acid |
| | salt, (3 exchangeable protons are not seen) |
| 28 | MS m/z 391.9, 393.8 [M + H]⁺; ¹H NMR (METHANOL-d₄) δ: 7.28-7.41 |
| | (m, 3H), 7.10-7.19 (m, 2H), 6.50-6.55 (m, 1H), 4.70-4.78 (m, 1H), 4.62- |
| | 4.70 (m, 2H), 4.08-4.18 (m, 1H), 3.61-3.76 (m, 1H), 3.33-3.37 (m, 1H), |
| | 2.26-2.39 (m, 1H), 1.87-1.99 (m, 2H), 1.71-1.85 (m, 1H). trifluoracetic |
| | acid salt, (3 exchangeable protons are not seen) |
| 29 | MS m/z 471.8 [M + H]⁺; ¹H NMR (METHANOL-d₄) δ: 7.27-7.40 (m, |
| | 2H), 7.09-7.20 (m, 2H), 6.51-6.58 (m, 1H), 4.96-5.04 (m, 1H), 4.59- |
| | 4.69 (m, 2H), 4.11-4.19 (m, 1H), 3.95-4.03 (m, 1H), 3.61-3.76 (m, 1H), |
| | 3.35-3.46 (m, 1H), 2.27-2.38 (m, 1H), 1.80-1.99 (m, 2H). trifluoracetic |
| | acid salt, (3 exchangeable protons are not seen) |
| 35 | MS m/z 392.1 [M + H]⁺; ¹HNMR(400 MHz, METHANOL-d₄) δ: 7.28 |
| | (dd, J = 5.1, 1.2 Hz, 1H), 7.06 (d, J = 2.5 Hz, 1H), 6.97 (dd, J = 5.1, 3.5 |
| | Hz, 1H), 6.49 (s, 1H), 4.74 (s, 2H), 3.06-2.86 (m, 1H), 2.35 (s, 3H), |
| | 2.02 (dd, J = 38.5, 12.1 Hz, 1H), 1.87 (s, 2H), 1.64-1.53 (m, 1H), |
| | 1.50-1.32 (m, 3H). (3 exchangeable protons are not seen) |
| 49 | MS m/z 459.9 [M + H]⁺; ¹H NMR (400 MHz, DMSO-d₆) δ: 8.00-7.97 |
| | (m, 1H), 7.41-7.40 (m, 1H), 7.10 (s, 1H), 7.00-6.98 (m, 1H), 6.63 (s, |
| | 1H), 4.73 (d, J = 4 Hz, 2H), 4.47 (d, J = 8 Hz, 1H), 3.96 (d, J = 12 Hz, |
| | 1H), 3.52-3.45 (m, 1H), 2.80-2.61 (m, 1H), 2.02 (d, J = 8 Hz, 1H), |
| | 1.50 (s, 2H), 1.47-1.43 (m, 1H), 1.24 (s, 1H), 0.86-0.83 (m, 1H). |
| 50 | MS m/z 392.0 [M + H]⁺; ¹HNMR(400 MHz, METHANOL-d₄) δ: 7.28 |
| | (dd, J = 5.1, 1.2 Hz, 1H), 7.06 (dd, J = 3.5, 1.0 Hz, 1H), 6.97 (dd, J = |
| | 5.1, 3.5 Hz, 1H), 6.50 (s, 1H), 4.74 (s, 2H), 3.08-2.91 (m, 2H), 2.35 (s, |
| | 3H), 2.03 (dd, J = 37.1, 12.3 Hz, 2H), 1.92-1.81 (m, 2H), 1.67-1.54 |
| | (m, 1H), 1.52-1.35 (m, 3H). (3 exchangeable protons are not seen) |
| 51 | MS m/z 392.0 [M + H]⁺; ¹HNMR(400 MHz, METHANOL-d₄) δ: 7.28 |
| | (dd, J = 5.1, 1.2 Hz, 1H), 7.06 (d, J = 2.5 Hz, 1H), 6.97 (dd, J = 5.1, 3.5 |
| | Hz, 1H), 6.49 (s, 1H), 4.74 (s, 2H), 3.06-2.86 (m, 2H), 2.35 (s, 3H), |
| | 2.02 (dd, J = 38.5, 12.1 Hz, 2H), 1.92-1.81 (m, 2H), 1.67-1.54 (m, |
| | 1H), 1.50-1.32 (m, 3H). (3 exchangeable protons are not seen) |
| 52 | MS m/z 457.9 [M + H]⁺; ¹HNMR(400 MHz, METHANOL-d₄) δ: 7.29 |
| | (dd, J = 5.1, 1.2 Hz, 1H), 7.08 (dd, J = 3.5, 1.0 Hz, 1H), 6.97 (dd, J = |
| | 5.1, 3.5 Hz, 1H), 6.56 (s, 1H), 4.75 (s, 2H), 3.59 (dt, J = 11.9, 3.5 Hz, |
| | 1H), 3.48 (dd, J = 5.7, 2.5 Hz, 1H), 1.92-1.48 (m, 8H). (3 |
| | exchangeable protons are not seen) |
| 53 | MS m/z 458.0 [M + H]⁺; ¹HNMR(400 MHz, METHANOL-d₄) δ: 7.29 |
| | (dd, J = 5.1, 1.2 Hz, 1H), 7.08 (dd, J = 3.4, 0.9 Hz, 1H), 6.97 (dd, J = |
| | 5.1, 3.5 Hz, 1H), 6.59 (s, 1H), 4.74 (d, J = 9.9 Hz, 2H), 3.73 (ddd, J = |
| | 35.7, 19.2, 14.8 Hz, 2H), 1.92-1.48 (m, 8H). (3 exchangeable protons |
| | are not seen) |
| 54 | MS m/z 458.0 [M + H]⁺; ¹H NMR (400 MHz, METHANOL-d₄) δ: 7.30 |
| | (dd, J = 5.1, 1.0 Hz, 1H), 7.08 (d, J = 2.6 Hz, 1H), 6.97 (dd, J = 5.0, 3.5 |
| | Hz, 1H), 6.56 (d, J = 12.8 Hz, 1H), 4.75 (s, 2H), 3.28-3.19 (m, 1H), |
| | 3.09 (d, J = 31.5 Hz, 1H), 1.92-1.48 (m, 8H). (3 exchangeable protons |
| | are not seen) |
| 55 | MS m/z 457.9 [M + H]⁺; ¹HNMR(400 MHz, METHANOL-d₄) δ: 7.29 |
| | (dd, J = 5.1, 1.2 Hz, 1H), 7.08 (dd, J = 3.5, 1.0 Hz, 1H), 6.97 (dd, J = |
| | 5.1, 3.5 Hz, 1H), 6.56 (s, 1H), 4.75 (s, 2H), 3.59 (dt, J = 11.9, 3.5 Hz, |
| | 1H), 3.48 (dd, J = 5.7, 2.5 Hz, 1H), 1.92-1.48 (m, 8H). (3 |
| | exchangeable protons are not seen) |
| 56 | MS m/z 459.9 [M + H]⁺; ¹H NMR (400 MHz, DMSO) δ: 7.98-7.97 (m, |
| | 1H), 7.41-7.40 (m, 1H), 7.11 (s, 1H), 7.00-6.98 (m, 1H), 6.63 (s, 1H), |
| | 4.73 (d, J = 4 Hz, 2H), 4.47 (d, J = 8 Hz, 1H), 3.97-3.94 (m, 1H), |
| | 3.52-3.45 (m, 1H), 2.80-2.65 (m, 1H), 2.02-1.99 (m, 1H), 1.75 (s, 2H), |
| | 1.52-1.48 (m, 1H), 1.24 (s, 2H). |
| 71 | MS m/z 392.0 [M + H]⁺; ¹HNMR(400 MHz, METHANOL-d₄) δ: 7.28 |
| | (dd, J = 5.1, 1.2 Hz, 1H), 7.06 (dd, J = 3.5, 1.0 Hz, 1H), 6.97 (dd, J = |
| | 5.1, 3.5 Hz, 1H), 6.50 (s, 1H), 4.74 (s, 2H), 3.08-2.91 (m, 2H), 2.35 (d, |
| | J = 4.4 Hz, 3H), 2.03 (dd, J = 37.1, 12.3 Hz, 2H), 1.92-1.81 (m, 2H), |
| | 1.67-1.54 (m, 1H), 1.52-1.35 (m, 3H). (3 exchangeable protons are |
| | not seen) |
| 76 | MS m/z 426.1 [M + H]⁺; ¹HNMR(400 MHz, CHLOROFORM-d) δ: 7.29 |
| | (d, J = 5.1 Hz, 1H), 7.07 (s, 1H), 7.01 (dd, J = 7.1, 3.6 Hz, 1H), 6.54 (s, |
| | 1H), 4.69 (d, J = 4.8 Hz, 2H), 3.75-3.62 (m, 1H), 3.46 (s, 1H), 1.97- |
| | 1.61 (m, 10H). (3 exchangeable protons are not seen) |
| 77 | MS m/z 426.1 [M + H]⁺; ¹HNMR(400 MHz, CHLOROFORM-d) δ: 7.29 |
| | (d, J = 5.0 Hz, 1H), 7.07 (s, 1H), 7.03-6.99 (m, 1H), 6.54 (s, 1H), 4.69 |
| | (d, J = 4.7 Hz, 2H), 3.64 (s, 1H), 3.44 (s, 1H), 1.97-1.61 (m, 10H). (3 |
| | exchangeable protons are not seen) |
| 78 | MS m/z 426.2 [M + H]⁺; ¹HNMR(400 MHz, CHLOROFORM-d) δ: 7.30 |
| | (d, J, J = 5.1 Hz, 1H), 7.08 (d, J = 3.2 Hz, 1H), 7.02 (dd, J = 5.0, 3.6 Hz, |
| | 1H), 6.55 (s, 1H), 4.70 (d, J = 4.7 Hz, 2H), 3.24 (t, J = 8.2 Hz, 1H), 3.11 |
| | (t, J = 7.2 Hz, 1H), 1.92-1.60 (m, 10H), 1.45 (s, 2H). (1 exchangeable |
| | protons are not seen) |
| 79 | MS m/z 426.2 [M + H]⁺; ¹HNMR(400 MHz, CHLOROFORM-d) δ: 7.23 |
| | (d, J = 4.9 Hz, 1H), 7.00 (s, 1H), 6.96-6.93 (m, 1H), 6.49 (s, 1H), 4.63 |
| | (d, J = 4.8 Hz, 2H), 3.17 (t, J = 8.4 Hz, 1H), 3.05 (d, J = 7.7 Hz, 1H), |
| | 1.83-1.55 (m, 10H), 1.36 (s, 2H). (1 exchangeable protons are not |
| | seen) |
| 81 | MS m/z 406.2 [M + H]⁺; ¹H NMR (400 MHz, DMSO-d₆) δ: 7.64 (t, J = |
| | 6.0 Hz, 1H), 7.38 (dd, J = 5.0, 1.1 Hz, 1H), 7.09 (d, J = 2.5 Hz, 1H), |
| | 6.98 (dd, J = 5.0, 3.5 Hz, 1H), 6.49 (s, 1H), 4.69 (d, J = 5.9 Hz, 2H), |
| | 2.98 (dt, J = 6.0, 3.0 Hz, 2H), 2.25 (s, 3H), 1.90-1.20 (m, 12H). |
| 82 | MS m/z 406.2 [M + H]⁺; ¹H NMR (400 MHz, DMSO-d₆) δ: 7.66 (t, J = |
| | 6.0 Hz, 1H), 7.40-7.36 (m, 1H), 7.08 (s, 1H), 6.97 (dd, J = 5.0, 3.5 Hz, |
| | 1H), 6.46 (s, 1H), 4.68 (d, J = 6.0 Hz, 2H), 2.98 (dt, J = 6.0, 3.0 Hz, |
| | 2H), 2.24 (s, 3H), 1.91-1.15 (m, 12H). |
| 83 | MS m/z 406.1 [M + H]⁺; ¹H NMR (400 MHz, DMSO-d₆) δ: 7.64 (s, 1H), |
| | 7.38 (dd, J = 5.1, 1.2 Hz, 1H), 7.09 (d, J = 2.5 Hz, 1H), 6.98 (dd, J = 5.0, |
| | 3.5 Hz, 1H), 6.48 (s, 1H), 4.69 (d, J = 6.0 Hz, 2H), 2.98 (dt, J = 6.0, 3.0 |
| | Hz, 2H), 2.25 (s, 3H), 1.85-1.42 (m, 12H). |
| 84 | MS m/z 406.1 [M + H]⁺; ¹H NMR (400 MHz, DMSO-d₆) δ: 7.66 (t, J = |
| | 6.1 Hz, 1H), 7.38 (dd, J = 5.1, 1.2 Hz, 1H), 7.08 (d, J = 2.4 Hz, 1H), |
| | 6.97 (dd, J = 5.0, 3.5 Hz, 1H), 6.46 (s, 1H), 4.68 (d, J = 5.9 Hz, 2H), |
| | 2.98 (dt, J = 6.0, 3.0 Hz, 2H), 2.24 (s, 3H), 2.00-1.40 (m, 12H). |
| 91 | MS m/z 440.0 [M + H]⁺; ¹HNMR(400 MHz, DMSO-d₆) δ: 7.96 (t, J = |
| | 6.1 Hz, 1H), 7.39 (dd, J = 5.1, 1.2 Hz, 1H), 7.10 (dd, J = 3.4, 1.0 Hz, |
| | 1H), 6.98 (dd, J = 5.1, 3.5 Hz, 1H), 6.56 (s, 1H), 4.70 (d, J = 6.0 Hz, |
| | 2H), 3.90-3.68 (m, 1H), 3.04 (d, J = 7.1 Hz, 1H), 2.08 (dd, J = 30.1, |
| | 19.2 Hz, 1H), 1.96-1.37 (m, 13H). |
| 92 | MS m/z 420.3 [M + H]⁺; ¹H NMR (DMSO-d₆) δ: 7.64 (t, J = 6.0 Hz, 1H), |
| | 7.38 (dd, J = 5.0, 1.1 Hz, 1H), 7.09 (d, J = 2.5 Hz, 1H), 6.98 (dd, J = 5.0, |
| | 3.5 Hz, 1H), 6.49 (s, 1H), 4.69 (d, J = 5.9 Hz, 2H), 2.98 (dt, J = 6.0, 3.0 |
| | Hz, 2H), 2.25 (s, 3H), 1.90-1.20 (m, 14H). |
| 93 | MS m/z 419.9 [M + H]⁺; ¹H NMR (DMSO-d₆) δ: 7.64 (s, 1H), 7.38 (dd, J = |
| | 5.1, 1.2 Hz, 1H), 7.09 (d, J = 2.5 Hz, 1H), 6.98 (dd, J = 5.0, 3.5 Hz, |
| | 1H), 6.48 (s, 1H), 4.69 (d, J = 6.0 Hz, 2H), 2.98 (dt, J = 6.0, 3.0 Hz, |
| | 2H), 2.25 (s, 3H), 1.85-1.42 (m, 14H). |
| 94 | MS m/z 419.9 [M + H]⁺; ¹H NMR (DMSO-d₆) δ: 7.66 (t, J = 6.0 Hz, 1H), |
| | 7.40-7.36 (m, 1H), 7.08 (s, 1H), 6.97 (dd, J = 5.0, 3.5 Hz, 1H), 6.46 (s, |
| | 1H), 4.68 (d, J = 6.0 Hz, 2H), 2.98 (dt, J = 6.0, 3.0 Hz, 2H), 2.24 (s, |
| | 3H), 1.91-1.15 (m, 14H). |
| 95 | MS m/z 420.3 [M + H]⁺; ¹H NMR (DMSO-d₆) δ: 7.66 (t, J = 6.1 Hz, 1H), |
| | 7.38 (dd, J = 5.1, 1.2 Hz, 1H), 7.08 (d, J = 2.4 Hz, 1H), 6.97 (dd, J = 5.0, |
| | 3.5 Hz, 1H), 6.46 (s, 1H), 4.68 (d, J = 5.9 Hz, 2H), 2.98 (dt, J = 6.0, 3.0 |
| | Hz, 2H), 2.25 (s, 3H), 2.00-1.40 (m, 14H). |
| 96 | MS m/z 440.0 [M + H]⁺; ¹HNMR(400 MHz, METHANOL-d₄) δ: 7.29 |
| | (dd, J = 5.1, 1.1 Hz, 1H), 7.07 (d, J = 3.4 Hz, 1H), 6.97 (dd, J = 5.1, 3.5 |
| | Hz, 1H), 6.56 (d, J = 8.4 Hz, 1H), 4.74 (s, 2H), 3.43 (d, J = 5.9 Hz, 2H), |
| | 2.03-1.62 (m, 12H). (3 exchangeable protons are not seen) |
| 97 | MS m/z 440.0 [M + H]⁺; ¹HNMR(400 MHz, METHANOL-d₄) δ: 7.29 |
| | (d, J = 5.1 Hz, 1H), 7.07 (d, J = 3.2 Hz, 1H), 6.97 (dd, J = 5.0, 3.5 Hz, |
| | 1H), 6.57 (s, 1H), 4.74 (s, 2H), 3.57-3.33 (m, 2H), 2.07-1.62 (m, |
| | 12H). (3 exchangeable protons are not seen) |
| 98 | MS m/z 440.0 [M + H]⁺; ¹HNMR(400 MHz, METHANOL-d₄) δ: 7.29 |
| | (d, J = 5.1 Hz, 1H), 7.07 (d, J = 3.4 Hz, 1H), 7.00-6.93 (m, |
| | 1H), 6.56 (d, J = 1.7 Hz, 1H), 4.85 (s, 2H), 4.74 (s, 2H), 3.54-3.41 |
| | (m, 2H), 1.99-1.60 (m, 12H). (3 exchangeable protons are not seen) |
| 99 | MS m/z 486.0 [M + H]⁺; ¹HNMR(400 MHz, METHANOL-d₄) δ: 7.30 |
| | (dd, J = 5.1, 1.1 Hz, 1H), 7.08 (d, J = 2.5 Hz, 1H), 6.97 (dd, J = 5.0, 3.5 |
| | Hz, 1H), 6.57 (s, 1H), 4.75 (s, 2H), 3.53 (d, J = 5.3 Hz, 1H), 3.38 (s, |
| | 1H), 1.99-1.60 (m, 12H). (3 exchangeable protons are not seen) |
| 100 | MS m/z 484.0 [M + H]⁺; ¹HNMR(400 MHz, METHANOL-d₄) δ: 7.29 |
| | (d, J = 5.1 Hz, 1H), 7.08 (d, J = 3.3 Hz, 1H), 7.00-6.95 (m, 1H), 6.58 |
| | (s, 1H), 4.75 (s, 2H), 3.56 (s, 1H), 3.40 (s, 1H), 1.82 (ddd, J = 72.5, |
| | 39.6, 6.2 Hz, 12H). (3 exchangeable protons are not seen) |
| 101 | MS m/z 486.0 [M + H]⁺; ¹HNMR(400 MHz, METHANOL-d₄) δ: 7.29 |
| | (d, J = 5.2 Hz, 1H), 7.08 (d, J = 2.9 Hz, 1H), 6.97 (dd, J = 5.1, 3.5 Hz, |
| | 1H), 6.59 (s, 1H), 4.75 (s, 2H), 4.02 (s, 1H), 3.61 (s, 1H), 1.82 (d, J = |
| | 74.1 Hz, 12H). (3 exchangeable protons are not seen) |
| 102 | MS m/z 486.0 [M + H]⁺; ¹HNMR(400 MHz, METHANOL-d₄) δ: 7.29 |
| | (dd, J = 5.1, 1.1 Hz, 1H), 7.08 (d, J = 2.5 Hz, 1H), 6.97 (dd, J = 5.1, 3.5 |
| | Hz, 1H), 6.58 (s, 1H), 4.75 (s, 2H), 4.01 (dd, J = 9.4, 5.7 Hz, 1H), 3.50 |
| | (s, 1H), 1.99-1.66 (m, 12H). (3 exchangeable protons are not seen) |
| 269 | MS m/z 494.0 [M + H]⁺; ¹H NMR (METHANOL-d₄) δ: 7.21 (d, J = 5.1 |
| | Hz, 1H), 7.00 (d, J = 3.4 Hz, 1H), 6.88 (dd, J = 5.1, 3.4 Hz, 1H), 6.55 (s, |
| | 1H), 4.68 (s, 2H), 4.00 (dd, J = 25.7, 11.6 Hz, 1H), 3.65-3.55 (m, 1H), |
| | 2.34-2.24 (m, 1H), 2.20-2.07 (m, 1H), 2.05-1.85 (m, 3H), 1.75- |
| | 1.60 (m, 1H). (3 Exchangeable protons not observed). |
| 338 | MS m/z 428.1 [M + H]⁺; ¹H NMR (METHANOL-d₄) δ: 7.28 (d, J = 4.6 |
| | Hz, 1H), 7.07 (s, 1H), 6.97 (d, J = 4.6 Hz, 1H), 6.51 (s, 1H), 4.75 (s, |
| | 2H), 3.50 (dd, J = 27.3, 11.0 Hz, 1H), 3.25-3.10 (m, 1H), 2.33 (s, 3H), |
| | 2.26-2.20 (m, 1H), 2.18-2.08 (m, 1H), 2.00-1.80 (m, 2H), 1.75- |
| | 1.65 (m, 1H), 1.50-1.40 (m, 1H). (3 Exchangeable protons not |
| | observed). |
| 359 | MS m/z 494.0 [M + H]⁺; ¹H NMR (METHANOL-d₄) δ: 7.21 (d, J = 5.1 |
| | Hz, 1H), 7.00 (d, J = 3.4 Hz, 1H), 6.88 (dd, J = 5.1, 3.4 Hz, 1H), 6.55 (s, |
| | 1H), 4.68 (s, 2H), 4.56-4.44 (m, 1H), 3.90 (d, J = 5.8 Hz, 1H), 2.34- |
| | 2.24 (m, 1H), 2.20-2.07 (m, 1H), 2.10-1.61 (m, 4H). (3 Exchangeable |
| | protons not observed). |
| 370 | MS m/z 448.0 [M + H]⁺; ¹H NMR (METHANOL-d₄) δ: 6.71 (s, 1H), |
| | 4.15-4.01 (m, 3H), 3.75-3.60 (m, 1H), 2.40-2.29 (m, 1H), 2.29- |
| | 2.21 (m, 1H), 2.15-1.96 (m, 2H), 1.86-1.67 (m, 5H). (3 |
| | Exchangeable protons not observed). |
| 383 | MS m/z 496.0 [M + H]⁺; ¹H NMR (METHANOL-d₄) δ: 6.59 (s, 1H), |
| | 4.06-3.91 (m, 3H), 3.68-3.58 (m, 1H), 2.25 (dt, J = 15.7, 7.1 Hz, |
| | 1H), 2.16 (d, J = 12.3 Hz, 1H), 2.05-1.88 (m, 2H), 1.76-1.68 (m, |
| | 4H), 1.68-1.57 (m, 1H). (3 Exchangeable protons not observed). |

### Example 2

### Preparation of Compound 67 and Compound 68

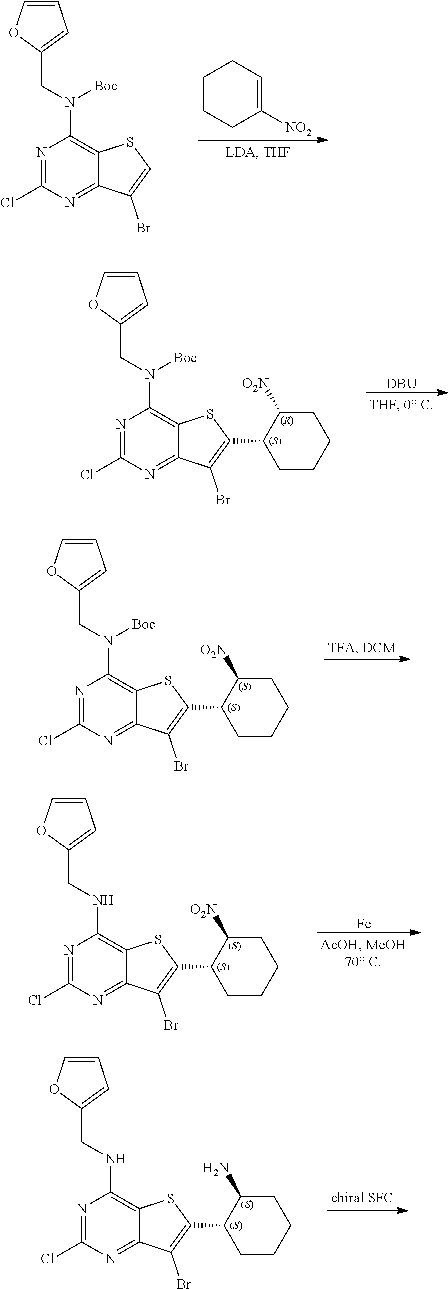

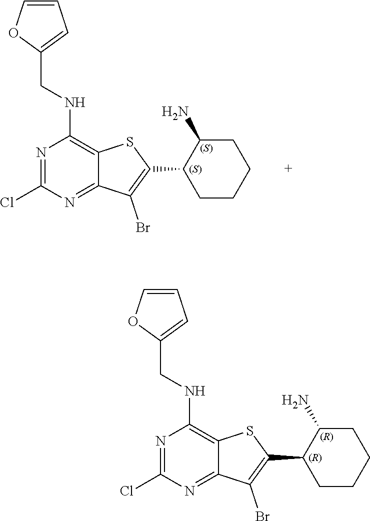

### Step 1: tert-Butyl (7-bromo-2-chloro-6-((1S,2R)-2-nitrocyclohexyl)thieno[3,2-d]pyrimidin-4-yl)(furan-2-ylmethyl)carbamate

To a solution of tert-butyl (7-bromo-2-chlorothieno[3,2-d]pyrimidin-4-yl)(furan-2-ylmethyl)carbamate (500 mg, 1.1 mmol) in THF (10 mL) was added lithium diisopropylamide (2M) in THF/heptane/ethylbenzene (0.8 mL, 1.7 mmol) at −78° C. The mixture was stirred at −78° C. for 1 hour. Then 1-nitrocyclohex-1-ene (185.7 mg, 1.5 mmol) was added, the mixture was stirred at −78° C. for 3 hours. TLC showed that most starting materials disappeared and new major spot. The reaction mixture was quenched with saturated ammonium chloride (125 mL) and the organic layer was extracted with EtOAc (125 ml×3). The organic phases were washed with brine, dried over Na₂SO₄, filtered, and concentrated in vacuo. The crude residue was purified by column chromatography on silica gel eluted with (EtOAc/PE=0 to 50%) to give the tert-butyl (7-bromo-2-chloro-6-((1S,2R)-2-nitrocyclohexyl)thieno[3,2-d]pyrimidin-4-yl)(furan-2-ylmethyl)carbamate (400 mg, 0.7 mmol, 62% Yield) as white solid. MS m/z 573.0 [M+H]⁺.

### Step 2: tert-Butyl (7-bromo-2-chloro-6-((1S,2S)-2-nitrocyclohexyl)thieno[3,2-d]pyrimidin-4-yl)(furan-2-ylmethyl)carbamate

To a solution of tert-butyl (7-bromo-2-chloro-6-((1S,2R)-2-nitrocyclohexyl)thieno[3,2-d]pyrimidin-4-yl)(furan-2-ylmethyl)carbamate (329 mg, 0.6 mmol) in THF (10 mL) was added DBU (88 mg, 0.6 mmol, 1 eq) at 0° C. The mixture was stirred at 0° C. for 3 hours. The mixture was poured into H₂O (50 mL). The organic layer was extracted with EtOAc (50 ml×3), washed with brine, dried over Na₂SO₄, filtered, and concentrated in vacuo. The crude residue was purified by column chromatography on silica gel eluted with (EtOAc/PE=0 to 50%) to give the tert-butyl (7-bromo-2-chloro-6-((1S,2S)-2-nitrocyclohexyl)thieno[3,2-d]pyrimidin-4-yl)(furan-2-ylmethyl)carbamate (221 mg, 0.4 mmol, 67% Yield) as a white solid. ¹H NMR (CHLOROFORM-d) δ ppm 7.29 (dd, J=1.8, 0.8 Hz, 1H), 6.32 (d, J=2.7 Hz, 1H), 6.29 (dd, J=3.2, 1.8 Hz, 1H), 5.19 (s, 2H), 4.89 (d, J=4.0 Hz, 1H), 3.95 (d, J=3.2 Hz, 1H), 2.50 (d, J=10.0 Hz, 1H), 2.19 (d, J=14.4 Hz, 1H), 2.10-1.80 (m, 3H), 1.80-1.40 (m, 3H), 1.53 (s, 9H).

### Step 3: 7-Bromo-2-chloro-N-(furan-2-ylmethyl)-6-((1S,2S)-2-nitrocyclohexyl)thieno[3,2-d]pyrimidin-4-amine

To a solution of tert-butyl (7-bromo-2-chloro-6-((1S,2S)-2-nitrocyclohexyl)thieno[3,2-d]pyrimidin-4-yl)(furan-2-ylmethyl)carbamate (221 mg, 0.4 mmol) in CH₂Cl₂ (4 mL) was added TFA (4 mL). The mixture was stirred at 25° C. for 1 hour and then concentrated under reduced pressure to afford the crude compound, purified by column chromatography on silica gel eluted with (MeOH/DCM=0 to 10%) to give the 7-bromo-2-chloro-N-(furan-2-ylmethyl)-6-((1S,2S)-2-nitrocyclohexyl)thieno[3,2-d]pyrimidin-4-amine (100 mg, 0.2 mmol, 55% Yield) as a yellow solid. MS m/z 470.9 [M+H]⁺.

### Step 4: 6-((1R,2R)-2-Aminocyclohexyl)-7-bromo-2-chloro-N-(furan-2-ylmethyl)thieno[3,2-d]pyrimidin-4-amine and 6-((1S,2S)-2-aminocyclohexyl)-7-bromo-2-chloro-N-(furan-2-ylmethyl)thieno[3,2-d]pyrimidin-4-amine

To a solution of 7-bromo-2-chloro-N-(2-furylmethyl)-6-[(1S,2S)-2-nitrocyclohexyl]thieno[3,2-d]pyrimidin-4-amine (400 mg, 0.8 mmol) in ethanol (6 mL) and acetic acid (2 mL, 34.9 mmol) was added Iron (785 mg, 14.0 mmol). The mixture was stirred at 65° C. for 18 hours. The reaction mixture was diluted by adding 25 mL H₂O. The organic layer was extracted with EtOAc (25 ml×3). The combined organic layers were washed with brine, dried over Na₂SO₄, filtered, and concentrated in vacuo. The crude residue was purified by column chromatography on silica gel eluted with (MeOH/DCM=0 to 10%) followed by purification by Prep-SFC (DAICELCHIRALPAK® OJ, Supercritical CO₂/MeOH (+0.1% 7.0 mol/l Ammonia in MeOH)/MeOH) to afford the 6-((1S,2S)-2-aminocyclohexyl)-7-bromo-2-chloro-N-(furan-2-ylmethyl)thieno[3,2-d]pyrimidin-4-amine (Compound 67, 26.4 mg, 18%) as off-white solid and 6-((1R,2R)-2-aminocyclohexyl)-7-bromo-2-chloro-N-(furan-2-ylmethyl)thieno[3,2-d]pyrimidin-4-amine (Compound 68, 26.3 mg, 18%) as off-white solid.

Compound 67: MS m/z 484.1 [M+H]⁺; ¹H NMR (METHANOL-d₄) δ ppm 7.43 (dd, J=1.8, 0.8 Hz, 1H), 6.40-6.30 (m, 2H), 4.75 (s, 2H), 3.30-3.20 (m, 1H), 3.10-2.85 (m, 1H), 2.10-2.00 (m, 2H), 2.00-1.80 (m, 2H), 1.60-1.00 (m, 4H) (3 exchangeable protons are not seen).

Compound 68: MS m/z 484.1 [M+H]⁺; ¹H NMR (METHANOL-d₄) δ ppm δ: 7.44-7.42 (m, 1H), 6.40-6.30 (m, 2H), 4.75 (s, 2H), 3.30-3.20 (m, 1H), 3.10-2.85 (m, 1H), 2.10-2.00 (m, 2H), 2.00-1.80 (m, 2H), 1.60-1.00 (m, 4H) (3 exchangeable protons are not seen).

The compounds below were prepared according to the procedure of Example 2 by substituting the appropriate starting materials, reagents, reaction conditions, and followed by chiral SFC resolution if needed.

| Compound | Spectral Data |
|---|---|
| 5 | MS m/z 378.0 [M + H]⁺; ¹H NMR (400 MHz, METHANOL-d₄) δ: 7.28 |
| | (dd, J = 5.1, 1.1 Hz, 1H), 7.05 (dd, J = 3.4, 1.0 Hz, 1H), 6.96 (dd, J = |
| | 5.1, 3.5 Hz, 1H), 6.47 (s, 1H), 4.73 (s, 2H), 3.37-3.31 (m, 2H), 2.35 (s, |
| | 3H), 2.21 (ddd, J = 15.5, 9.7, 4.2 Hz, 2H), 1.77 (dddd, J = 20.4, 17.2, |
| | 12.6, 8.3 Hz, 4H). (3 exchangeable protons are not seen) |
| 6 | MS m/z 378.0 [M + H]⁺; ¹H NMR (400 MHz METHANOL-d₄) δ: 7.28 |
| | (dd, J = 5.1, 1.1 Hz, 1H), 7.05 (d, J = 3.4 Hz, 1H), 6.96 (dd, J = 5.0, 3.5 |
| | Hz, 1H), 6.47 (s, 1H), 4.73 (s, 2H), 3.34 (s, 2H), 2.35 (s, 3H), 2.25 (ddd, |
| | J = 15.5, 9.7, 4.2 Hz, 2H), 1.75 (dddd, J = 20.4, 17.2, 12.6, 8.3 Hz, 4H). |
| | (3 exchangeable protons are not seen) |
| 7 | MS m/z 444.1 [M + H]⁺; 1H NMR (400 MHz, METHANOL-d₄) δ: 7.29 |
| | (dd, J = 5.1, 1.2 Hz, 1H), 7.12 (s, 1H), 7.08-7.04 (m, 1H), 6.97 (dd, J = |
| | 5.1, 3.5 Hz, 1H), 6.48 (s, 1H), 4.74 (s, 2H), 3.20-3.00 (m, 2H), 2.40- |
| | 2.10 (m, 2H), 2.00-1.80 (m, 2H), 1.65-1.50 (m, 1H). (3 exchangeable |
| | protons are not seen) |
| 8 | MS m/z 444.1 [M + H]⁺; ¹H NMR (400 MHz, METHANOL-d₄) δ: 7.29 |
| | (d, J = 5.2 Hz, 1H), 7.11 (s, 1H), 7.07 (d, J = 3.4 Hz, 1H), 6.99-6.94 |
| | (m, 1H), 6.49 (d, J = 1.6 Hz, 1H), 4.74 (s, 2H), 3.60-3.40 (m, 2H), |
| | 2.25-1.95 (m, 3H), 1.85-1.60 (m, 2H). (3 exchangeable protons are not |
| | seen) |
| 11 | MS m/z 392.1 [M + H]⁺; ¹HNMR(400 MHz, METHANOL-d₄) δ: 7.28 |
| | (dd, J = 5.1, 1.2 Hz, 1H), 7.06 (d, J = 2.4 Hz, 1H), 6.97 (dd, J = 5.1, 3.5 |
| | Hz, 1H), 6.47 (s, 1H), 4.73 (s, 2H), 3.39 (d, J = 8.5 Hz, 2H), 2.94-2.84 |
| | (m, 2H), 2.31-2.18 (m, 2H), 1.95-1.82 (m, 3H), 1.64 (s, 1H), 1.19 (t, |
| | J = 7.5 Hz, 3H). (3 exchangeable protons are not seen). |
| 12 | MS m/z 392.2 [M + H]⁺; ¹HNMR(400 MHz, METHANOL-d₄) δ: 7.28 |
| | (d, J = 5.0 Hz, 1H), 7.06 (d, J = 2.5 Hz, 1H), 6.97 (dd, J = 5.0, 3.6 Hz, |
| | 1H), 6.47 (d, J = 4.3 Hz, 1H), 4.73 (s, 2H), 3.38 (s, 2H), 2.88 (dd, J = |
| | 10.0, 7.4 Hz, 2H), 2.31-2.16 (m, 2H), 1.84 (dd, J = 27.4, 15.7 Hz, 3H), |
| | 1.63 (s, 1H), 1.19 (t, J = 7.5 Hz, 3H). (3 exchangeable protons are not |
| | seen). |
| 33 | MS m/z 377.9 [M + H]⁺; ¹H NMR (DMSO-d₆) δ: 7.51 (br t, J = 5.9 Hz, |
| | 1H), 7.22-7.30 (m, 2H), 7.06-7.20 (m, 2H), 6.32 (s, 1H), 5.69 (s, 1H), |
| | 4.46-4.53 (m, 2H), 4.32 (d, J = 9.0 Hz, 1H), 3.84-3.91 (m, 1H), 3.36-3.45 |
| | (m, 1H), 2.50-2.60 (m, 1H), 2.25 (s, 3H), 1.89-1.97 (m, 1H), 1.57-1.65 |
| | (m, 2H), 1.30-1.43 (m, 1H), 1.24 (br s, 2H). |
| 34 | MS m/z 411.9 [M + H]⁺; ¹H NMR (400 MHz, CHLOROFORM-d) δ: |
| | 7.30 (dd, J = 5.1, 1.2 Hz, 1H), 7.08 (d, J = 2.6 Hz, 1H), 7.01 (dd, J = 5.1, |
| | 3.5 Hz, 1H), 6.55 (s, 1H), 4.72 (dd, J = 17.4, 5.1 Hz, 2H), 3.08 (td, J = |
| | 11.6, 3.6 Hz, 1H), 2.81 (td, J = 10.5, 3.8 Hz, 1H), 2.13-1.96 (m, 2H), |
| | 1.92-1.78 (m, 2H), 1.33 (ddd, J = 20.4, 12.3, 9.3 Hz, 4H). (3 |
| | exchangeable protons are not seen). |
| 36 | MS m/z 413.7 [M + H]⁺; ¹H NMR (400 MHz, CHLOROFORM-d) δ: |
| | 7.30 (dd, J = 5.1, 1.2 Hz, 1H), 7.08 (dd, J = 3.4, 1.0 Hz, 1H), 7.01 (dd, J = |
| | 5.1, 3.5 Hz, 1H), 6.55 (s, 1H), 4.72 (dd, J = 13.3, 4.8 Hz, 2H), 3.13- |
| | 3.05 (m, 1H), 2.85-2.78 (m, 1H), 2.13-1.96 (m, 2H), 1.92-1.78 (m, |
| | 2H), 1.33 (ddd, J = 20.4, 12.3, 9.3 Hz, 4H). (3 exchangeable protons are |
| | not seen). |
| 39 | MS m/z 406.2 [M + H]⁺; ¹HNMR(400 MHz, METHANOL-d₄) δ: 7.28 |
| | (d, J = 4.9 Hz, 1H), 7.06 (s, 1H), 7.00-6.92 (m, 1H), 6.47 (s, 1H), 4.73 |
| | (s, 2H), 2.90 (dt, J = 15.8, 9.5 Hz, 4H), 2.07-1.81 (m, 4H), 1.45 (ddd, J = |
| | 34.9, 33.5, 22.2 Hz, 4H), 1.19 (t, J = 7.4 Hz, 3H). (3 exchangeable |
| | protons are not seen) |
| 40 | MS m/z 406.2 [M + H]⁺; ¹HNMR(400 MHz, METHANOL-d₄) δ: 7.28 |
| | (d, J = 5.1 Hz, 1H), 7.06 (s, 1H), 7.00-6.93 (m, 1H), 6.52 (s, 1H), 4.75 |
| | (s, 2H), 3.20 (t, J = 9.6 Hz, 2H), 2.90 (ddd, J = 59.8, 13.9, 7.1 Hz, 2H), |
| | 2.18 (s, 1H), 2.00 (dd, J = 53.6, 14.6 Hz, 3H), 1.72 (d, J = 12.4 Hz, 1H), |
| | 1.53 (t, J = 9.2 Hz, 3H), 1.20 (t, J = 7.4 Hz, 3H). (3 exchangeable |
| | protons are not seen) |
| 42 | MS m/z 445.0 [M + H]⁺; ¹HNMR(400 MHz, METHANOL-d₄) δ: 7.43 (s, |
| | 1H), 6.35 (q, J = 3.6 Hz, 2H), 4.75 (d, J = 4.5 Hz, 2H), 4.69 (d, J = 9.4 |
| | Hz, 1H), 4.04 (dd, J = 11.4, 4.2 Hz, 1H), 3.58 (td, J = 11.6, 3.1 Hz, 1H), |
| | 2.82 (td, J = 11.3, 4.1 Hz, 1H), 2.15 (d, J = 12.6 Hz, 1H), 1.82 (dd, J = |
| | 14.3, 10.4 Hz, 2H), 1.57 (dd, J = 11.9, 4.5 Hz, 1H). (3 exchangeable |
| | protons are not seen). |
| 43 | MS m/z 387.1 [M + H]⁺; ¹HNMR(400 MHz, METHANOL-d₄) δ: 7.37 |
| | (d, J = 7.3 Hz, 2H), 7.33-7.28 (m, 2H), 7.23 (dd, J = 8.3, 6.1 Hz, 1H), |
| | 4.81-4.71 (m, 2H), 3.03-2.95 (m, 1H), 2.84 (td, J = 10.5, 3.9 Hz, |
| | 1H), 2.33 (s, 3H), 2.08-1.76 (m, 4H), 1.60-1.27 (m, 4H). (3 |
| | exchangeable protons are not seen) |
| 44 | MS m/z 393.9 [M + H]⁺; ¹HNMR(400 MHz, METHANOL-d₄) δ: 7.28 |
| | (dd, J = 5.1, 1.2 Hz, 1H), 7.06 (dd, J = 3.5, 1.0 Hz, 1H), 6.96 (dd, J = |
| | 5.1, 3.5 Hz, 1H), 6.48 (s, 1H), 5.05 (s, 1H), 4.74 (s, 2H), 4.18-4.08 (m, |
| | 1H), 3.73 (dd, J = 11.6, 9.4 Hz, 1H), 3.22 (s, 1H), 2.32 (s, 3H), 2.01 |
| | (ddd, J = 19.4, 12.4, 6.2 Hz, 3H), 1.48 (d, J = 11.2 Hz, 1H). (3 |
| | exchangeable protons are not seen) |
| 45 | MS m/z 393.9 [M + H]⁺; ¹HNMR(400 MHz, METHANOL-d₄) δ: 7.28 |
| | (dd, J = 5.1, 1.2 Hz, 1H), 7.06 (dd, J = 3.5, 1.0 Hz, 1H), 6.96 (dd, J = |
| | 5.1, 3.5 Hz, 1H), 6.48 (s, 1H), 5.04 (d, J = 1.2 Hz, 1H), 4.74 (s, 2H), |
| | 4.13 (dd, J = 10.6, 3.4 Hz, 1H), 3.72 (dd, J = 11.6, 9.3 Hz, 1H), 3.20 (s, |
| | 1H), 2.32 (s, 3H), 2.15-1.89 (m, 3H), 1.55-1.39 (m, 1H). (3 |
| | exchangeable protons are not seen). |
| 48 | MS m/z 387.1 [M + H]⁺; ¹HNMR(400 MHz, METHANOL-d₄) δ: 7.37 |
| | (d, J = 7.2 Hz, 2H), 7.31 (dd, J = 10.0, 4.7 Hz, 2H), 7.26-7.20 (m, 1H), |
| | 4.81-4.70 (m, 2H), 3.09-3.00 (m, 1H), 2.93 (td, J = 10.5, 3.7 Hz, |
| | 1H), 2.33 (s, 3H), 1.94 (ddd, J = 26.5, 24.0, 11.4 Hz, 4H), 1.62-1.34 |
| | (m, 4H). (3 exchangeable protons are not seen) |
| 57 | MS m/z 503.7 [M + H]⁺; ¹HNMR(400 MHz, METHANOL-d₄) δ: 7.29 |
| | (dd, J = 5.1, 1.0 Hz, 1H), 7.07 (d, J = 2.6 Hz, 1H), 6.97 (dd, J = 5.0, 3.5 |
| | Hz, 1H), 6.56 (s, 1H), 4.75 (s, 2H), 3.28-3.20 (m, 1H), 3.12 (s, 1H), |
| | 2.06 (dd, J = 27.5, 12.8 Hz, 2H), 1.88 (t, J = 12.9 Hz, 2H), 1.64-1.41 |
| | (m, 4H). (3 exchangeable protons are not seen). |
| 58 | MS m/z 503.8 [M + H]⁺; ¹HNMR(400 MHz, METHANOL-d₄) δ: 7.29 |
| | (d, J = 4.9 Hz, 1H), 7.07 (d, J = 3.1 Hz, 1H), 6.99-6.95 (m, 1H), 6.55 |
| | (s, 1H), 4.74 (s, 2H), 3.25-3.14 (m, 1H), 3.05 (d, J = 6.9 Hz, 1H), 2.04 |
| | (dd, J = 25.5, 13.1 Hz, 2H), 1.85 (d, J = 11.0 Hz, 2H), 1.58-1.36 (m, |
| | 4H). (3 exchangeable protons are not seen) |
| 59 | MS m/z 377.1 [M + H]⁺; ¹HNMR(400 MHz, METHANOL-d₄) δ: 7.44- |
| | 7.39 (m, 1H), 6.37-6.29 (m, 2H), 4.79-4.69 (m, 2H), 3.14-2.96 (m, |
| | 2H), 2.33 (s, 3H), 2.09 (d, J = 10.5 Hz, 1H), 2.00 (d, J = 10.5 Hz, 1H), |
| | 1.89 (d, J = 20.1 Hz, 2H), 1.62-1.40 (m, 4H). (3 exchangeable protons |
| | are not seen). |
| 60 | MS m/z 377.1 [M + H]⁺; ¹HNMR(400 MHz, METHANOL-d₄) δ: 7.42 |
| | (d, J = 1.0 Hz, 1H), 6.36-6.30 (m, 2H), 4.79-4.68 (m, 2H), 3.08 (td, J = |
| | 11.2, 3.5 Hz, 1H), 2.99 (t, J = 10.2 Hz, 1H), 2.33 (s, 3H), 2.08 (d, J = |
| | 12.1 Hz, 1H), 1.99 (d, J = 11.7 Hz, 1H), 1.94-1.83 (m, 2H), 1.65- |
| | 1.37 (m, 4H). (3 exchangeable protons are not seen) |
| 61 | MS m/z 379.1 [M + H]⁺; ¹HNMR(400 MHz, METHANOL-d₄) δ: 7.42 |
| | (dd, J = 1.8, 0.8 Hz, 1H), 6.34 (dt, J = 8.8, 2.5 Hz, 2H), 4.80-4.69 (m, |
| | 2H), 4.61 (d, J = 9.4 Hz, 1H), 4.09-4.01 (m, 1H), 3.60 (td, J = 11.6, 3.1 |
| | Hz, 1H), 2.96 (td, J = 11.4, 4.1 Hz, 1H), 2.39 (s, 3H), 2.24-2.14 (m, |
| | 1H), 1.84 (dt, J = 24.7, 8.7 Hz, 2H), 1.61 (ddd, J = 24.4, 12.6, 4.7 Hz, |
| | 1H). (3 exchangeable protons are not seen). |
| 62 | MS m/z 379.1 [M + H]⁺; ¹HNMR(400 MHz, METHANOL-d₄) δ: 7.42 |
| | (dd, J = 1.8, 0.8 Hz, 1H), 6.33 (ddd, J = 3.8, 3.2, 1.3 Hz, 2H), 4.79- |
| | 4.69 (m, 2H), 4.51 (d, J = 9.3 Hz, 1H), 4.06-3.99 (m, 1H), 3.58 (td, J = |
| | 11.7, 2.9 Hz, 1H), 2.80 (ddd, J = 11.3, 9.3, 4.1 Hz, 1H), 2.39 (s, 3H), |
| | 2.13 (d, J = 10.3 Hz, 1H), 1.90-1.71 (m, 2H), 1.53 (ddd, J = 24.1, 12.8, |
| | 4.5 Hz, 1H). (3 exchangeable protons are not seen) |
| 63 | MS m/z 389.1 [M + H]⁺; ¹HNMR(400 MHz, METHANOL-d₄) δ: 7.40- |
| | 7.35 (m, 2H), 7.33-7.28 (m, 2H), 7.26-7.21 (m, 1H), 4.76 (q, J = 15.1 |
| | Hz, 2H), 4.51 (d, J = 9.3 Hz, 1H), 4.06-3.99 (m, 1H), 3.58 (td, J = |
| | 11.6, 2.9 Hz, 1H), 2.80 (ddd, J = 11.3, 9.3, 4.1 Hz, 1H), 2.38 (d, J, J = |
| | 3.0 Hz, 3H), 2.17-2.09 (m, 1H), 1.89-1.72 (m, 2H), 1.59-1.47 (m, |
| | 1H). (3 exchangeable protons are not seen) |
| 64 | MS m/z 389.1 [M + H]⁺; ¹HNMR(400 MHz, METHANOL-d₄) δ: 7.40- |
| | 7.35 (m, 2H), 7.33-7.28 (m, 2H), 7.26-7.21 (m, 1H), 4.82-4.74 (m, |
| | 2H), 4.70 (d, J = 9.5 Hz, 1H), 4.11-4.04 (m, 1H), 3.63 (td, J = 11.5, 3.4 |
| | Hz, 1H), 3.13 (ddd, J = 11.6, 9.7, 4.1 Hz, 1H), 2.40 (d, J = 4.8 Hz, 3H), |
| | 2.28-2.20 (m, 1H), 1.86 (dd, J = 11.0, 3.9 Hz, 2H), 1.70 (ddd, J = 24.4, |
| | 12.3, 4.7 Hz, 1H). (3 exchangeable protons are not seen) |
| 65 | MS m/z 453.0 [M + H]⁺; ¹H NMR (400 MHz, CHLOROFORM-d) δ: |
| | 7.40-7.32 (m, 5H), 5.31 (s, 1H), 4.83 (t, J = 7.4 Hz, 2H), 3.11 (dd, J = |
| | 14.6, 6.9 Hz, 1H), 2.83 (td, J = 10.5, 3.9 Hz, 1H), 2.03 (t, J = 12.0 Hz, |
| | 2H), 1.83 (d, J = 8.0 Hz, 2H), 1.41-1.22 (m, 4H). (2 exchangeable |
| | protons are not seen) |
| 66 | MS m/z 453.1 [M + H]⁺; ¹H NMR (400 MHz, CHLOROFORM-d) δ: |
| | 7.35 (dt, J = 10.7, 4.4 Hz, 5H), 5.30 (s, 1H), 4.84 (d, J = 5.6 Hz, 2H), |
| | 3.11 (dd, J = 14.6, 6.8 Hz, 1H), 2.83 (td, J = 10.4, 3.9 Hz, 1H), 2.03 (t, J = |
| | 12.3 Hz, 2H), 1.84 (s, 2H), 1.45-1.22 (m, 4H). (2 exchangeable |
| | protons are not seen) |
| 69 | MS m/z 455.0 [M + H]⁺; ¹HNMR(400 MHz, CHLOROFORM-d) δ: 7.40- |
| | 7.31 (m, 5H), 5.30 (s, 1H), 4.85 (d, J = 5.5 Hz, 2H), 4.62 (d, J = 9.2 |
| | Hz, 1H), 4.09 (dd, J = 11.5, 4.5 Hz, 1H), 3.56 (td, J = 11.8, 2.8 Hz, 1H), |
| | 2.84-2.68 (m, 1H), 2.18 (d, J = 10.8 Hz, 1H), 1.82 (dt, J = 30.8, 11.4 |
| | Hz, 2H). (3 exchangeable protons are not seen). |
| 70 | MS m/z 455.0 [M + H]⁺; ¹HNMR(400 MHz, CHLOROFORM-d) δ: 7.39- |
| | 7.30 (m, 5H), 5.30 (s, 1H), 4.85 (d, J = 5.5 Hz, 2H), 4.62 (d, J, J = 9.2 |
| | Hz, 1H), 4.09 (dd, J = 11.6, 4.6 Hz, 1H), 3.56 (td, J = 11.8, 2.7 Hz, 1H), |
| | 2.84-2.74 (m, 1H), 2.18 (d, J = 12.8 Hz, 1H), 1.90-1.75 (m, 2H). (3 |
| | exchangeable protons are not seen). |
| 85 | MS m/z 484.0 [M + H]⁺; ¹H NMR (DMSO-d₆) δ: 8.19 (s, 0.8H, formic |
| | acid), 7.96 (t, J = 6.0 Hz, 1H), 7.34 (d, J = 5.1 Hz, 1H), 7.05 (d, J = 3.4 |
| | Hz, 1H), 6.92 (dd, J = 5.1, 3.4 Hz, 1H), 6.54 (s, 1H), 4.66 (d, J = 6.0 Hz, |
| | 2H), 3.64 (dd, J = 6.4, 3.4 Hz, 1H), 3.33-3.25 (m, 2H), 2.33-2.26 (m, |
| | 1H), 2.26-2.19 (m, 1H), 1.83-1.76 (m, 1H), 1.76-1.54 (m, 6H), 1.54- |
| | 1.43 (m, 1H), 1.15-1.05 (m, 1H). MS m/z 484.0 [M + H]⁺; |
| 86 | MS m/z 420.1 [M + H]⁺; ¹HNMR(400 MHz, METHANOL-d₄) δ: 7.28 |
| | (d, J = 4.2 Hz, 1H), 7.06 (d, J = 2.6 Hz, 1H), 6.96 (dd, J = 5.0, 3.6 Hz, |
| | 1H), 6.47 (s, 1H), 4.73 (s, 2H), 3.11 (s, 2H), 2.89 (dt, J = 14.2, 6.5 Hz, |
| | 2H), 1.96-1.55 (m, 10H), 1.18 (t, J = 7.5 Hz, 3H) (3 exchangeable |
| | protons are not seen) |
| 87 | MS m/z 420.1 [M + H]⁺; ¹HNMR(400 MHz, METHANOL-d₄) δ: 7.28 |
| | (dd, J = 5.1, 1.1 Hz, 1H), 7.06 (d, J = 2.5 Hz, 1H), 6.96 (dd, J = 5.0, 3.5 |
| | Hz, 1H), 6.47 (s, 1H), 4.73 (s, 2H), 3.14-3.08 (m, 2H), 2.89 (dt, J = |
| | 14.0, 6.4 Hz, 2H), 1.96-1.55 (m, 10H), 1.18 (t, J = 7.5 Hz, 3H) (3 |
| | exchangeable protons are not seen). |
| 107 | MS m/z 372.1 [M + H]⁺; ¹HNMR(400 MHz, METHANOL-d₄) δ: 7.37- |
| | 7.30 (m, 4H), 7.25 (t, J = 6.5 Hz, 1H), 6.30 (s, 1H), 4.56 (s, 2H), 3.37- |
| | 3.32 (m, 2H), 2.35 (s, 3H), 2.29-2.13 (m, 2H), 1.95-1.78 (m, 3H), |
| | 1.67-1.56 (m, 2H). (3 exchangeable protons are not seen). |
| 108 | MS m/z 454.0 [M + H]⁺; ¹H NMR (400 MHz, METHANOL-d₄) δ: 7.34 |
| | (d, J = 6.7 Hz, 5H), 6.41 (s, 1H), 4.69 (d, J = 9.3 Hz, 1H), 4.58 (s, 2H), |
| | 4.06 (d, J = 11.2 Hz, 1H), 3.60 (dt, J = 11.6, 5.9 Hz, 1H), 2.87 (s, 3H), |
| | 2.16 (d, J = 10.1 Hz, 1H), 1.81 (d, J = 11.9 Hz, 2H), 1.59-1.50 (m, 2H) |
| | (3 exchangeable protons are not seen) |
| 109 | MS m/z 388.1 [M + H]⁺; ¹HNMR(400 MHz, METHANOL-d₄) δ: 7.37- |
| | 7.30 (m, 4H), 7.27-7.21 (m, 1H), 6.34 (s, 1H), 4.57 (s, 2H), 4.52 (d, J = |
| | 9.3 Hz, 1H), 4.04 (dd, J = 11.3, 4.5 Hz, 1H), 3.60 (td, J = 11.7, 2.8 Hz, |
| | 1H), 2.86 (s, 3H), 2.15 (d, J = 12.7 Hz, 1H), 1.81 (dt, J = 21.4, 11.1 Hz, |
| | 2H), 1.59-1.50 (m, 2H) (3 exchangeable protons are not seen) |
| 110 | MS m/z 408.1 [M + H]⁺; ¹HNMR(400 MHz, METHANOL-d₄) δ: 7.38- |
| | 7.31 (m, 4H), 7.26 (t, J = 6.4 Hz, 1H), 6.42 (s, 1H), 4.68 (d, J = 9.4 Hz, |
| | 1H), 4.58 (s, 2H), 4.09-4.01 (m, 1H), 3.60 (td, J = 11.6, 3.0 Hz, 1H), |
| | 2.86 (td, J = 11.3, 4.1 Hz, 1H), 2.17 (d, J = 10.3 Hz, 1H), 1.91-1.74 |
| | (m, 2H), 1.58 (ddd, J = 24.3, 12.6, 4.5 Hz, 1H). (3 exchangeable protons |
| | are not seen) |
| 113 | MS m/z 454.0 [M + H]⁺; ¹H NMR (400 MHz, METHANOL-d₄) δ: 7.32 |
| | (dt, J = 23.5, 6.9 Hz, 5H), 6.41 (s, 1H), 4.69 (d, J = 9.4 Hz, 1H), 4.58 (s, |
| | 2H), 4.05 (d, J = 11.5 Hz, 1H), 3.59 (s, 1H), 2.87 (td, J = 11.2, 4.0 Hz, |
| | 1H), 2.16 (d, J = 10.9 Hz, 1H), 1.80 (s, 2H), 1.63-1.50 (m, 1H). (3 |
| | exchangeable protons are not seen). |
| 114 | MS m/z 382.0 [M + H]⁺; ¹HNMR(400 MHz, METHANOL-d₄) δ: 7.45 (s, |
| | 1H), 6.64 (s, 1H), 6.34 (d, J = 14.1 Hz, 2H), 4.54 (s, 2H), 3.52-3.44 |
| | (m, 2H), 2.22 (brs, 2H), 1.93 (brs, 1H), 1.65 (brs, 3H). (3 exchangeable |
| | protons are not seen) |
| 115 | MS m/z 444.1 [M + H]⁺; ¹HNMR(400 MHz, METHANOL-d₄) δ: 7.45 (s, |
| | 1H), 6.66 (s, 1H), 6.35 (d, J = 12.2 Hz, 2H), 4.72 (d, J = 9.4 Hz, 1H), |
| | 4.54 (s, 2H), 4.06 (d, J = 9.0 Hz, 1H), 3.60 (t, J = 11.0 Hz, 1H), 2.93 (s, |
| | 1H), 2.16 (s, 1H), 1.81 (s, 2H), 1.61 (d, J = 12.4 Hz, 1H). (3 |
| | exchangeable protons are not seen). |
| 116 | MS m/z 398.2 [M + H]⁺; ¹HNMR(400 MHz, METHANOL-d₄) δ: 7.45 |
| | (d, J = 1.0 Hz, 1H), 6.64 (d, J = 9.5 Hz, 1H), 6.37-6.34 (m, 1H), 6.33 |
| | (d, J = 2.7 Hz, 1H), 4.67 (d, J = 9.4 Hz, 1H), 4.54 (s, 2H), 4.07-4.01 |
| | (m, 1H), 3.59 (td, J = 11.6, 3.0 Hz, 1H), 3.33 (d, J = 12.3 Hz, 1H), 2.90- |
| | 2.76 (m, 1H), 2.16 (d, J = 10.6 Hz, 1H), 1.83 (dd, J = 24.4, 20.5 Hz, |
| | 2H). (3 exchangeable protons are not seen) |
| 117 | MS m/z 382.0 [M + H]⁺; ¹HNMR(400 MHz, METHANOL-d₄) δ: 7.45 (s, |
| | 1H), 6.64 (s, 1H), 6.34 (d, J = 14.1 Hz, 2H), 4.54 (s, 2H), 3.52-3.44 |
| | (m, 2H), 2.22 (brs, 2H), 1.93 (brs, 1H), 1.65 (brs, 3H). (3 exchangeable |
| | protons are not seen) |
| 118 | MS m/z 444.1 [M + H]⁺; ¹HNMR(400 MHz, METHANOL-d₄) δ: 7.45 |
| | (d, J = 1.0 Hz, 1H), 6.65 (s, 1H), 6.38-6.30 (m, 2H), 4.67 (d, J = 9.4 |
| | Hz, 1H), 4.54 (s, 2H), 4.07-3.99 (m, 1H), 3.59 (td, J = 11.6, 3.0 Hz, |
| | 1H), 2.88-2.80 (m, 1H), 2.15 (d, J = 10.9 Hz, 1H), 1.89-1.75 (m, |
| | 2H), 1.56 (ddd, J = 24.2, 12.6, 4.6 Hz, 1H). (3 exchangeable protons are |
| | not seen) |
| 119 | MS m/z 398.2 [M + H]⁺; ¹HNMR(400 MHz, METHANOL-d₄) δ: 7.45 (s, |
| | 1H), 6.71 (s, 1H), 6.42-6.28 (m, 2H), 4.98 (s, 1H), 4.55 (s, 2H), 4.13 |
| | (d, J = 11.1 Hz, 1H), 3.66 (t, J = 8.9 Hz, 1H), 3.37 (dd, J = 10.6, 4.0 Hz, |
| | 1H), 2.31 (d, J = 11.6 Hz, 1H), 1.85 (dd, J = 20.3, 8.8 Hz, 3H). (3 |
| | exchangeable protons are not seen) |
| 120 | MS m/z 362.1 [M + H]⁺; ¹HNMR(400 MHz, METHANOL-d₄) δ: 7.44 |
| | (d, J = 1.1 Hz, 1H), 6.53 (s, 1H), 6.35 (dd, J = 3.2, 1.9 Hz, 1H), 6.29 (d, |
| | J = 2.6 Hz, 1H), 4.52 (s, 2H), 3.35 (brs, 1H), 2.35 (s, 3H), 2.29-2.13 |
| | (m, 2H), 1.96-1.75 (m, 4H), 1.58 (d, J = 9.3 Hz, 1H). (3 exchangeable |
| | protons are not seen) |
| 122 | MS m/z 532.1 [M + H]⁺; ¹HNMR(400 MHz, METHANOL-d₄) δ: 7.29 |
| | (dd, J = 5.1, 1.1 Hz, 1H), 7.07 (d, J = 2.5 Hz, 1H), 6.97 (dd, J = 5.1, 3.5 |
| | Hz, 1H), 6.54 (s, 1H), 4.74 (s, 2H), 4.00 (d, J = 11.2 Hz, 1H), 3.35 (s, |
| | 1H), 2.26 (d, J = 8.3 Hz, 1H), 2.01-1.62 (m, 11H). (3 exchangeable |
| | protons are not seen) |
| 123 | MS m/z 532.1 [M + H]⁺; ¹HNMR(400 MHz, METHANOL-d₄) δ: 7.29 |
| | (d, J = 5.0 Hz, 1H), 7.07 (d, J = 3.1 Hz, 1H), 7.00-6.95 (m, 1H), 6.54 |
| | (s, 1H), 4.74 (s, 2H), 4.00 (d, J = 11.3 Hz, 1H), 3.35 (brs, 1H), 2.26 (d, J = |
| | 8.4 Hz, 1H), 1.78 (td, J = 34.6, 17.7 Hz, 11H). (3 exchangeable |
| | protons are not seen) |
| 126 | MS m/z 388.1 [M + H]⁺; ¹HNMR(400 MHz, METHANOL-d₄) δ: 7.38- |
| | 7.22 (m, 5H), 6.34 (s, 1H), 4.57 (s, 2H), 4.52 (d, J = 9.3 Hz, 1H), 4.04 |
| | (d, J = 11.2 Hz, 1H), 3.65-3.54 (m, 1H), 2.91-2.81 (m, 1H), 2.42 (s, |
| | 3H), 2.15 (d, J = 12.8 Hz, 1H), 1.81 (dd, J = 29.4, 13.3 Hz, 2H), 1.54 |
| | (ddd, J = 24.3, 12.9, 4.5 Hz, 1H). (3 exchangeable protons are not seen) |
| 132 | MS m/z 500.1 [M + H]⁺; ¹H NMR (400 MHz, METHANOL-d₄) δ: 7.37- |
| | 7.31 (m, 4H), 7.26 (d, J = 6.6 Hz, 1H), 6.39 (s, 1H), 4.69 (s, 1H), 4.57 |
| | (s, 2H), 4.07 (d, J = 9.1 Hz, 1H), 3.61 (t, J = 11.3 Hz, 1H), 3.08 (d, J = |
| | 42.5 Hz, 1H), 2.17 (s, 1H), 1.81 (s, 2H), 1.60 (s, 1H). (3 exchangeable |
| | protons are not seen) |
| 133 | MS m/z 378.0 [M + H]⁺; ¹HNMR(400 MHz, METHANOL-d₄) δ: 7.44 |
| | (d, J = 1.1 Hz, 1H), 6.56 (d, J = 8.9 Hz, 1H), 6.35 (dd, J = 3.2, 1.8 Hz, |
| | 1H), 6.30 (d, J = 2.5 Hz, 1H), 4.53-4.49 (m, 3H), 4.03 (dd, J = 11.4, |
| | 4.4 Hz, 1H), 3.59 (d, J = 2.8 Hz, 1H), 2.85 (ddd, J = 11.2, 9.3, 4.1 Hz, |
| | 1H), 2.41 (s, 3H), 2.14 (d, J = 12.6 Hz, 1H), 1.80 (d, J = 16.2 Hz, 2H), |
| | 1.53 (dd, J = 11.5, 4.4 Hz, 1H). (3 exchangeable protons are not seen) |
| 134 | MS m/z 378.0 [M + H]⁺; ¹HNMR(400 MHz, METHANOL-d₄) δ: 7.44 |
| | (d, J = 1.1 Hz, 1H), 6.57 (s, 1H), 6.38-6.33 (m, 1H), 6.30 (d, J = 3.1 |
| | Hz, 1H), 4.53 (t, J = 4.6 Hz, 3H), 4.02 (s, 1H), 3.60 (d, J = 2.8 Hz, 1H), |
| | 2.88 (d, J = 2.0 Hz, 1H), 2.41 (s, 3H), 2.15 (d, J = 11.3 Hz, 1H), 1.85 (d, |
| | J = 50.0 Hz, 2H), 1.55 (dd, J = 11.6, 4.5 Hz, 1H). (3 exchangeable |
| | protons are not seen) |
| 136 | MS m/z 500.2 [M + H]⁺; ¹HNMR(400 MHz, METHANOL-d₄) δ: 7.37- |
| | 7.31 (m, 4H), 7.29-7.24 (m, 1H), 6.44 (s, 1H), 4.96 (d, J = 9.8 Hz, |
| | 1H), 4.58 (s, 2H), 4.13 (d, J = 9.9 Hz, 1H), 3.67 (dd, J = 11.4, 8.1 Hz, |
| | 1H), 3.46-3.38 (m, 1H), 2.36 (d, J = 14.9 Hz, 1H), 1.95-1.78 (m, |
| | 3H). (3 exchangeable protons are not seen) |
| 143 | MS m/z 445.0 [M + H]⁺; ¹HNMR(400 MHz, METHANOL-d₄) δ: 7.45- |
| | 7.39 (m, 1H), 6.35 (dd, J = 4.1, 2.3 Hz, 2H), 4.75 (d, J = 5.5 Hz, 2H), |
| | 4.67 (d, J = 9.3 Hz, 1H), 4.02 (d, J = 4.6 Hz, 1H), 3.61-3.53 (m, 1H), |
| | 2.79 (dd, J = 4.1, 1.9 Hz, 1H), 2.14 (d, J = 11.6 Hz, 1H), 1.78 (s, 2H), |
| | 1.62-1.50 (m, 1H). (3 exchangeable protons are not seen) |
| 145 | MS m/z 362.1 [M + H]⁺; ¹HNMR(400 MHz, METHANOL-d₄) δ: 7.46- |
| | 7.42 (m, 1H), 6.53 (s, 1H), 6.35 (dd, J = 3.2, 1.9 Hz, 1H), 6.31-6.26 |
| | (m, 1H), 4.51 (s, 2H), 3.33 (s, 1H), 2.36 (d, J = 7.4 Hz, 3H), 2.29-2.23 |
| | (m, 1H), 2.18-2.12 (m, 1H), 1.92-1.77 (m, 4H), 1.62 (d, J = 9.7 Hz, |
| | 1H). (3 exchangeable protons are not seen) |
| 154 | MS m/z 489.7 [M + H]⁺; ¹HNMR(400 MHz, METHANOL-d₄) δ: 7.29 |
| | (d, J = 4.9 Hz, 1H), 7.07 (s, 1H), 7.01-6.95 (m, 1H), 6.59 (s, 1H), 4.75 |
| | (s, 2H), 3.82 (dd, J = 15.2, 7.8 Hz, 2H), 2.41 (dd, J = 14.1, 7.4 Hz, 2H), |
| | 1.96 (ddd, J = 29.8, 14.9, 7.1 Hz, 6H). (3 exchangeable protons are not |
| | seen) |
| 155 | MS m/z 428.0 [M + H]⁺; ¹HNMR(400 MHz, METHANOL-d₄) δ: 7.45 (s, |
| | 1H), 6.62 (s, 1H), 6.34 (d, J = 14.6 Hz, 2H), 4.53 (s, 2H), 3.55-3.39 |
| | (m, 2H), 2.34 (d, J = 10.8 Hz, 1H), 2.19 (d, J = 7.3 Hz, 1H), 1.95-1.74 |
| | (m, 3H), 1.61 (d, J = 10.1 Hz, 1H). (3 exchangeable protons are not |
| | seen) |
| 156 | MS m/z 408.1 [M + H]⁺; ¹HNMR(400 MHz, METHANOL-d₄) δ: 7.38- |
| | 7.31 (m, 4H), 7.29-7.23 (m, 1H), 6.43 (s, 1H), 4.76 (d, J, J = 9.5 Hz, |
| | 1H), 4.58 (s, 2H), 4.08 (dd, J = 12.4, 3.1 Hz, 1H), 3.62 (td, J = 11.5, 3.1 |
| | Hz, 1H), 2.99 (td, J = 11.3, 4.1 Hz, 1H), 2.21 (d, J = 10.0 Hz, 1H), 1.90- |
| | 1.76 (m, 2H), 1.65 (ddd, J = 24.5, 12.4, 4.4 Hz, 1H). (3 exchangeable |
| | protons are not seen) |
| 157 | MS m/z 518.0 [M + H]⁺; ¹HNMR(400 MHz, METHANOL-d₄) δ: 7.29 |
| | (dd, J = 5.1, 1.2 Hz, 1H), 7.07 (d, J = 2.4 Hz, 1H), 6.97 (dd, J = 5.1, 3.5 |
| | Hz, 1H), 6.55 (s, 1H), 4.74 (s, 2H), 3.37 (s, 2H), 1.88 (dd, J = 24.5, 11.0 |
| | Hz, 6H), 1.77-1.67 (m, 4H). (3 exchangeable protons are not seen) |
| 158 | MS m/z 434.1 [M + H]⁺; ¹HNMR(400 MHz, METHANOL-d₄) δ: 7.28 |
| | (dd, J = 5.1, 1.1 Hz, 1H), 7.06 (d, J = 2.5 Hz, 1H), 6.97 (dd, J = 5.1, 3.5 |
| | Hz, 1H), 6.48 (s, 1H), 4.73 (s, 2H), 3.34 (s, 2H), 2.89 (dt, J = 13.9, 6.3 |
| | Hz, 2H), 2.04-1.84 (m, 6H), 1.67 (d, J = 19.5 Hz, 6H), 1.20 (t, J = 7.5 |
| | Hz, 3H). (3 exchangeable protons are not seen) |
| 159 | MS m/z 392.1 [M + H]⁺; ¹HNMR(400 MHz, METHANOL-d₄) δ: 7.34 |
| | (dd, J = 9.0, 5.3 Hz, 4H), 7.27 (d, J, J = 6.3 Hz, 1H), 6.40 (s, 1H), 4.57 |
| | (s, 2H), 3.52-3.39 (m, 2H), 2.34 (d, J = 7.7 Hz, 1H), 2.20 (d, J = 7.4 |
| | Hz, 1H), 2.03 (s, 1H), 1.90 (dd, J = 14.0, 6.3 Hz, 2H), 1.62 (d, J = 9.2 |
| | Hz, 1H). (3 exchangeable protons are not seen) |
| 160 | MS m/z 372.1 [M + H]⁺; ¹HNMR(400 MHz, METHANOL-d₄) δ: 7.37- |
| | 7.30 (m, 4H), 7.29-7.22 (m, 1H), 6.35 (s, 1H), 4.56 (s, 2H), 3.65- |
| | 3.56 (m, 2H), 2.39-2.30 (m, 5H), 2.02-1.90 (m, 3H), 1.78 (s, 1H). (3 |
| | exchangeable protons are not seen) |
| 161 | MS m/z 438.0 [M + H]⁺; ¹HNMR(400 MHz, METHANOL-d₄) δ: 7.42- |
| | 7.25 (m, 5H), 6.40 (s, 1H), 4.57 (s, 2H), 3.61-3.47 (m, 2H), 2.44- |
| | 2.33 (m, 1H), 2.23 (dd, J = 12.7, 5.2 Hz, 1H), 1.98-1.83 (m, 3H), 1.69 |
| | (dd, J = 21.6, 8.0 Hz, 1H). (3 exchangeable protons are not seen) |
| 162 | MS m/z 489.7 [M + H]⁺; ¹HNMR(400 MHz, METHANOL-d₄) δ: 7.29 |
| | (dd, J = 5.1, 1.1 Hz, 1H), 7.07 (d, J = 2.5 Hz, 1H), 6.97 (dd, J = 5.1, 3.5 |
| | Hz, 1H), 6.54 (s, 1H), 4.74 (s, 2H), 3.50 (t, J = 6.4 Hz, 2H), 2.37 (dd, J = |
| | 12.3, 4.7 Hz, 1H), 2.25-2.14 (m, 1H), 1.99-1.78 (m, 3H), 1.64 (d, J = |
| | 8.7 Hz, 1H). (3 exchangeable protons are not seen) |
| 163 | MS m/z 518.0 [M + H]⁺; ¹HNMR(400 MHz, METHANOL-d₄) δ: 7.29 |
| | (d, J = 5.1 Hz, 1H), 7.08 (d, J = 3.4 Hz, 1H), 7.00-6.94 (m, 1H), 6.59 |
| | (s, 1H), 4.75 (s, 2H), 3.56 (s, 2H), 2.05-1.89 (m, 6H), 1.73 (s, 4H). (3 |
| | exchangeable protons are not seen) |
| 164 | MS m/z 434.1 [M + H]⁺; ¹HNMR(400 MHz, METHANOL-d₄) δ: 7.28 |
| | (dd, J = 5.1, 1.1 Hz, 1H), 7.06 (d, J = 2.5 Hz, 1H), 6.97 (dd, J = 5.0, 3.5 |
| | Hz, 1H), 6.47 (s, 1H), 4.73 (s, 2H), 3.27 (t, J = 4.1 Hz, 2H), 2.89 (dd, J = |
| | 12.1, 7.5 Hz, 2H), 2.00-1.74 (m, 8H), 1.63 (d, J = 7.1 Hz, 4H), 1.20 (t, |
| | J = 7.5 Hz, 3H). (3 exchangeable protons are not seen) |
| 165 | MS m/z 428.0 [M + H]⁺; ¹HNMR(400 MHz, METHANOL-d₄) δ: 7.45 (s, |
| | 1H), 6.61 (s, 1H), 6.38-6.28 (m, 2H), 4.53 (s, 2H), 3.53-3.38 (m, |
| | 2H), 2.38-2.28 (m, 1H), 2.17 (dt, J = 12.6, 6.2 Hz, 1H), 1.96-1.74 |
| | (m, 3H), 1.60 (dd, J = 18.9, 10.1 Hz, 1H). (3 exchangeable protons are |
| | not seen) |
| 166 | MS m/z 438.1 [M + H]⁺; ¹HNMR(400 MHz, METHANOL-d₄) δ: 7.32 |
| | (dt, J = 23.5, 6.9 Hz, 5H), 6.41 (s, 1H), 4.69 (d, J = 9.4 Hz, 1H), 4.58 (s, |
| | 2H), 4.05 (d, J = 11.5 Hz, 1H), 3.59 (s, 1H), 2.87 (td, J = 11.2, 4.0 Hz, |
| | 1H), 2.16 (d, J = 10.9 Hz, 1H), 1.80 (s, 2H), 1.63-1.50 (m, 1H). (3 |
| | exchangeable protons are not seen) |
| 167 | MS m/z 392.1 [M + H]⁺; ¹HNMR(400 MHz, METHANOL-d₄) δ: 7.34 |
| | (d, J = 5.8 Hz, 4H), 7.26 (s, 1H), 6.41 (s, 1H), 4.57 (s, 2H), 3.56-3.45 |
| | (m, 2H), 2.35 (d, J = 7.0 Hz, 1H), 2.22 (d, J = 10.9 Hz, 1H), 2.04 (brs, |
| | 1H), 1.91 (brs, 2H), 1.66 (brs, 1H). (3 exchangeable protons are not |
| | seen) |
| 173 | MS m/z 395.9 [M + H]⁺; ¹HNMR(400 MHz, METHANOL-d₄) δ: 7.44 (s, |
| | 1H), 6.64 (d, J = 2.9 Hz, 1H), 6.34 (d, J = 12.7 Hz, 2H), 4.54 (s, 2H), |
| | 3.13 (s, 1H), 2.91 (d, J = 10.5 Hz, 1H), 2.03 (dd, J = 26.6, 12.4 Hz, 2H), |
| | 1.86 (s, 2H), 1.60-1.35 (m, 4H). (3 exchangeable protons are not seen) |
| 174 | MS m/z 398.0 [M + H]⁺; ¹HNMR(400 MHz, METHANOL-d₄) δ: 7.45 (s, |
| | 1H), 6.66 (s, 1H), 6.38-6.31 (m, 2H), 4.54 (s, 2H), 4.07 (d, J = 11.8 |
| | Hz, 1H), 3.99 (dd, J = 10.9, 3.6 Hz, 1H), 3.60 (t, J = 11.0 Hz, 1H), 3.51- |
| | 3.38 (m, 2H), 3.19 (t, J = 20.6 Hz, 1H), 2.00 (d, J = 9.4 Hz, 1H), 1.74- |
| | 1.62 (m, 1H). (3 exchangeable protons are not seen) |
| 179 | MS m/z 414.0 [M + H]⁺; ¹HNMR(400 MHz, METHANOL-d₄) δ: 7.30 |
| | (d, J = 4.1 Hz, 1H), 7.08 (d, J = 2.6 Hz, 1H), 6.98 (dd, J = 5.0, 3.6 Hz, |
| | 1H), 6.59 (s, 1H), 4.76 (s, 2H), 4.18-3.95 (m, 2H), 3.60 (t, J = 12.1 Hz, |
| | 1H), 3.51-3.39 (m, 2H), 3.20 (d, J = 10.0 Hz, 1H), 2.01 (d, J = 11.2 |
| | Hz, 1H), 1.68 (dd, J = 21.4, 10.0 Hz, 1H). (3 exchangeable protons are |
| | not seen) |
| 197 | MS m/z 453.1 [M + H]⁺; ¹HNMR(400 MHz, METHANOL-d₄) δ: 8.51 |
| | (d, J = 5.6 Hz, 2H), 7.44 (d, J = 5.2 Hz, 2H), 6.39 (s, 1H), 4.69 (s, 2H), |
| | 3.26 (dd, J = 17.4, 8.5 Hz, 1H), 3.06 (t, J = 8.7 Hz, 1H), 2.09 (dd, J = |
| | 23.0, 12.5 Hz, 2H), 1.89 (d, J = 11.1 Hz, 2H), 1.69-1.45 (m, 4H). (3 |
| | exchangeable protons are not seen) |
| 201 | MS m/z 477.0 [M + H]⁺; ¹HNMR(400 MHz, METHANOL-d₄) δ: 7.45- |
| | 7.41 (m, 1H), 6.37-6.32 (m, 2H), 4.82-4.70 (m, 2H), 3.78 (dd, J = |
| | 28.1, 12.0 Hz, 1H), 3.13 (s, 1H), 2.21 (s, 1H), 2.12-1.91 (m, 3H), 1.71 |
| | (dd, J = 16.7, 11.9 Hz, 1H), 1.54-1.43 (m, 1H)., (3 exchangeable |
| | protons are not seen) |
| 215 | MS m/z 493.9 [M + H]⁺; ¹HNMR(400 MHz, METHANOL-d₄) δ: 8.20 |
| | (dd, J = 59.8, 56.2 Hz, 1H), 7.31-6.91 (m, 3H), 5.08-4.91 (m, 3H), |
| | 4.17 (d, J = 11.2 Hz, 1H), 3.63 (t, J = 11.9 Hz, 1H), 3.19 (d, J = 47.9 Hz, |
| | 1H), 2.39-2.22 (m, 1H), 2.11-1.66 (m, 3H). (3 exchangeable protons |
| | are not seen) |
| 218 | MS m/z 408.1 [M + H]⁺; ¹HNMR(400 MHz, METHANOL-d₄) δ: 7.39- |
| | 7.31 (m, 4H), 7.27 (d, J = 6.3 Hz, 1H), 6.44 (s, 1H), 4.58 (s, 2H), 4.10 |
| | (d, J = 7.4 Hz, 1H), 4.05-3.98 (m, 1H), 3.62 (t, J = 11.2 Hz, 1H), 3.57- |
| | 3.45 (m, 2H), 3.34 (s, 1H), 2.05 (d, J = 11.0 Hz, 1H), 1.75 (dd, J = |
| | 11.3, 7.4 Hz, 1H). (3 exchangeable protons are not seen) |
| 224 | MS m/z 493.0 [M + H]⁺; ¹H NMR (METHANOL-d₄) δ: 8.15 (dd, J = |
| | 60.9, 56.5 Hz, 1H), 7.27-7.21 (m, 1H), 7.02 (d, J = 2.8 Hz, 1H), 6.90 |
| | (dd, J = 5.0, 3.6 Hz, 1H), 6.61 (s, 1H), 4.69-4.61 (m, 3H), 4.07-4.01 |
| | (m, 1H), 3.49 (t, J = 11.0 Hz, 1H), 2.92 (t, J = 8.6 Hz, 1H), 2.13 (d, J = |
| | 12.1 Hz, 1H), 2.06-1.74 (m, 1H), 1.69 (d, J = 13.1 Hz, 1H), 1.60- |
| | 1.36 (m, 1H). (3, exchangeable protons not observed). |
| 225 | MS m/z 523.9 [M + H]⁺; ¹HNMR(400 MHz, METHANOL-d₄) δ: 8.26 |
| | (dd, J = 60.5, 56.5 Hz, 1H), 7.44 (s, 1H), 6.45-6.26 (m, 2H), 4.80 (t, J = |
| | 9.1 Hz, 2H), 4.71 (d, J = 10.1 Hz, 1H), 4.13 (dd, J = 11.3, 4.4 Hz, 1H), |
| | 3.56 (t, J = 10.9 Hz, 1H), 2.93 (td, J = 10.5, 3.9 Hz, 1H), 2.21 (d, J = |
| | 10.6 Hz, 1H), 1.94 (dt, J = 13.2, 9.0 Hz, 1H), 1.77 (d, J = 13.9 Hz, 1H), |
| | 1.54 (ddd, J = 16.9, 13.1, 3.9 Hz, 1H). (3 exchangeable protons are not |
| | seen) |
| 231 | MS m/z 376.9 [M + H]⁺; ¹HNMR(400 MHz, METHANOL-d₄) δ: 7.44 |
| | (d, J = 1.0 Hz, 1H), 6.54 (s, 1H), 6.35 (dd, J = 3.2, 1.9 Hz, 1H), 6.30 (d, |
| | J = 2.6 Hz, 1H), 4.52 (s, 2H), 3.01-2.92 (m, 1H), 2.84 (td, J = 10.5, 3.8 |
| | Hz, 1H), 2.34 (s, 3H), 2.04 (dd, J = 13.8, 4.4 Hz, 1H), 1.99-1.91 (m, |
| | 1H), 1.85 (t, J = 10.7 Hz, 2H), 1.57-1.32 (m, 4H). (3 exchangeable |
| | protons are not seen) |
| 232 | MS m/z 394.1 [M + H]⁺; ¹HNMR(400 MHz, METHANOL-d₄) δ: 7.28 |
| | (d, J = 5.0 Hz, 1H), 7.06 (d, J = 2.7 Hz, 1H), 6.99-6.94 (m, 1H), 6.50 |
| | (s, 1H), 4.74 (s, 2H), 4.07 (d, J = 7.6 Hz, 1H), 3.94 (dd, J = 11.5, 4.2 Hz, |
| | 1H), 3.60 (t, J = 11.0 Hz, 1H), 3.46 (t, J = 11.2 Hz, 1H), 3.28-3.19 (m, |
| | 1H), 3.13 (s, 1H), 2.38 (d, J = 4.2 Hz, 3H), 2.07-1.93 (m, 1H), 1.74- |
| | 1.59 (m, 1H). (3 exchangeable protons are not seen) |
| 237 | MS m/z 406.2 [M + H]⁺; ¹HNMR(400 MHz, METHANOL-d₄) δ: 7.38- |
| | 7.32 (m, 4H), 7.29-7.23 (m, 1H), 6.40 (s, 1H), 4.57 (s, 2H), 3.19- |
| | 3.11 (m, 1H), 2.95-2.87 (m, 1H), 2.04 (dd, J = 23.0, 13.1 Hz, 2H), 1.86 |
| | (t, J = 9.5 Hz, 2H), 1.61 (d, J = 12.4 Hz, 1H), 1.43 (dt, J = 27.5, 12.0 Hz, |
| | 3H). (3 exchangeable protons are not seen) |
| 243 | MS m/z 491.0 [M + H]⁺; ¹H NMR (METHANOL-d₄) δ: 7.43 (s, 1H), |
| | 6.38-6.33 (m, 2H), 4.99 (d, J = 9.8 Hz, 1H), 4.83-4.69 (m, 2H), 4.16- |
| | 4.10 (m, 1H), 3.67 (td, J = 11.4, 2.8 Hz, 1H), 3.41 (td, J = 10.6, 4.0 |
| | Hz, 1H), 2.34-2.28 (m, 1H), 1.98-1.80 (m, 3H). (3 Exchangeable |
| | protons not observed). |
| 244 | MS m/z 432.0 [M + H]⁺; ¹HNMR(400 MHz, METHANOL-d₄) δ: 8.17 |
| | (dd, J = 59.4, 56.3 Hz, 1H), 7.44 (s, 1H), 6.36 (d, J = 1.2 Hz, 2H), 4.98 |
| | (d, J = 10.5 Hz, 1H), 4.80 (d, J = 15.5 Hz, 2H), 4.20 (dd, J = 11.6, 4.4 |
| | Hz, 1H), 3.67 (t, J = 11.1 Hz, 1H), 3.49 (d, J = 9.6 Hz, 1H), 2.36 (d, J = |
| | 13.6 Hz, 1H), 2.06 (d, J = 13.0 Hz, 1H), 1.85 (dd, J = 24.0, 13.4 Hz, |
| | 2H). (3 exchangeable protons are not seen) |
| 248 | MS m/z 493.8 [M + H]⁺; ¹HNMR(400 MHz, METHANOL-d₄) δ: 8.22 |
| | (dd, J = 60.3, 56.6 Hz, 1H), 7.31-6.91 (m, 3H), 5.06-4.93 (m, 2H), |
| | 4.74 (dd, J = 21.5, 10.1 Hz, 1H), 4.12 (dd, J = 11.4, 4.3 Hz, 1H), 3.54 |
| | (dd, J = 28.0, 17.6 Hz, 1H), 3.15-2.84 (m, 1H), 2.19 (dd, J = 18.3, 13.5 |
| | Hz, 1H), 1.93 (t, J = 12.7 Hz, 1H), 1.77 (d, J = 14.1 Hz, 1H), 1.55 (dd, J = |
| | 24.2, 9.2 Hz, 1H). (3 exchangeable protons are not seen) |
| 251 | MS m/z 454.9 [M + H]⁺; ¹HNMR(400 MHz, METHANOL-d₄) δ: 8.50- |
| | 8.47 (m, 2H), 7.43-7.40 (m, 2H), 6.39 (s, 1H), 4.77 (d, J, J = 8.0 Hz, 1H), |
| | 4.68 (s, 2H), 4.12-4.04 (m, 1H), 3.66-3.68 (m, 1H), 3.04-2.95 (m, |
| | 1H), 2.25-2.17 (m, 1H), 1.91-1.79 (m, 2H), 1.70-1.60 (m, 1H). (3 |
| | exchangeable protons are not seen) |
| 254 | MS m/z 540.0 [M + H]⁺; ¹HNMR(400 MHz, METHANOL-d₄) δ: 8.24 |
| | (dd, J = 60.6, 56.5 Hz, 1H), 7.28 (dd, J = 5.1, 1.1 Hz, 1H), 7.11 (d, J = |
| | 2.8 Hz, 1H), 6.95 (dd, J = 5.1, 3.5 Hz, 1H), 5.00 (t, J = 9.6 Hz, 2H), 4.70 |
| | (d, J = 10.1 Hz, 1H), 4.13 (d, J = 6.8 Hz, 1H), 3.56 (t, J = 10.9 Hz, 1H), |
| | 2.93 (dd, J = 10.3, 6.7 Hz, 1H), 2.21 (d, J = 12.7 Hz, 1H), 1.94 (d, J = |
| | 12.9 Hz, 1H), 1.77 (d, J = 12.9 Hz, 1H), 1.61-1.45 (m, 1H). (3 |
| | exchangeable protons are not seen) |
| 257 | MS m/z 457.95 [M + H]⁺; ¹H NMR (METHANOL-d₄) δ: 7.20 (br d, |
| | J = 4.9 Hz, 1H), 6.98 (br s, 1H), 6.88 (br s, 1H), 6.21-6.66 (m, 2H), 5.07 |
| | (br s, 2H), 4.66 (s, 2H), 3.22-3.29 (m, 1H), 2.98-3.18 (m, 1H), 1.96-2.20 |
| | (m, 2H), 1.74-1.93 (m, 2H), 1.31-1.63 (m, 4H). (3 exchangeable protons |
| | are not seen) |
| 260 | MS m/z 378.0 [M + H]⁺; ¹HNMR (400 MHz, METHANOL-d₄) δ: 7.44 |
| | (s, 1H), 6.56 (s, 1H), 6.37-6.28 (m, 2H), 4.52 (s, 2H), 4.07 (d, J = 8.0 |
| | Hz, 1H), 3.94 (dd, J = 11.4, 4.4 Hz, 1H), 3.60 (s, 1H), 3.46 (t, J = 11.2 |
| | Hz, 1H), 3.25 (d, J, J = 4.2 Hz, 1H), 3.13 (s, 1H), 2.38 (s, 3H), 1.98 (d, J = |
| | 10.8 Hz, 1H), 1.67 (d, J = 7.5 Hz, 1H). (3 exchangeable protons are |
| | not seen) |
| 262 | MS m/z 447.0 [M + H]⁺; ¹HNMR(400 MHz, METHANOL-d₄) δ: 8.19 |
| | (dd, J = 60.4, 56.6 Hz, 1H), 7.34 (dd, J = 5.1, 1.1 Hz, 1H), 7.12 (d, J = |
| | 3.1 Hz, 1H), 7.00 (dd, J = 5.1, 3.5 Hz, 1H), 6.73 (s, 1H), 4.83 (s, 21H), |
| | 4.79 (s, 2H), 4.17 (dd, J = 11.5, 4.3 Hz, 1H), 3.63 (td, J = 12.2, 2.1 Hz, |
| | 1H), 2.30 (d, J = 13.1 Hz, 1H), 2.01 (dd, J = 9.1, 3.9 Hz, 1H), 1.88- |
| | 1.64 (m, 2H). (3 exchangeable protons are not seen) |
| 265 | MS m/z 507.0 [M + H]⁺; ¹HNMR(400 MHz, METHANOL-d₄) δ: 7.76 |
| | (d, J = 2.8 Hz, 1H), 7.53 (s, 1H), 6.51 (s, 1H), 4.88 (s, 2H), 4.04 (dd, J = |
| | 14.6, 10.3 Hz, 2H), 3.55 (t, J = 11.1 Hz, 1H), 3.46-3.36 (m, 1H), 3.21 |
| | (dd, J = 26.2, 8.8 Hz, 2H), 1.94 (dd, J = 33.6, 19.7 Hz, 2H). (3 |
| | exchangeable protons are not seen) |
| 266 | MS m/z 506.0 [M + H]⁺; ¹H NMR (METHANOL-d₄) δ: 7.30 (dd, J = 5.1, |
| | 1.3 Hz, 1H), 7.08 (dd, J = 3.5, 1.3 Hz, 1H), 6.98 (dd, J = 5.1, 3.5 Hz, |
| | 1H), 6.62 (s, 1H), 4.99 (d, J = 9.8 Hz, 1H), 4.77 (s, 3H), 4.18-4.10 (m, |
| | 1H), 3.68 (td, J = 11.2, 3.6 Hz, 1H), 3.44 (dd, J = 10.0, 3.9 Hz, 1H), |
| | 2.36-2.25 (m, 1H), 1.98-1.79 (m, 3H). (3 Exchangeable protons not |
| | observed). |
| 270 | MS m/z 476.9 [M + H]⁺; ¹HNMR(400 MHz, METHANOL-d₄) δ: 8.24 |
| | (dd, J = 61.1, 56.5 Hz, 1H), 7.48 (s, 1H), 6.74 (s, 1H), 6.48-6.31 (m, |
| | 2H), 4.71 (d, J = 10.1 Hz, 1H), 4.59 (s, 2H), 4.13 (dd, J = 11.4, 4.9 Hz, |
| | 1H), 3.55 (dd, J = 30.9, 20.5 Hz, 1H), 2.97 (s, 1H), 2.21 (d, J = 12.2 Hz, |
| | 1H), 1.95 (d, J = 13.1 Hz, 1H), 1.78 (d, J = 12.7 Hz, 1H), 1.55 (d, J = |
| | 14.7 Hz, 1H). (3 exchangeable protons are not seen) |
| 271 | MS m/z 432.0 [M + H]⁺; ¹HNMR(400 MHz, METHANOL-d₄) δ: 8.17 |
| | (dd, J = 59.4, 56.3 Hz, 1H), 7.44 (s, 1H), 6.36 (d, J = 1.2 Hz, 2H), 4.98 |
| | (d, J = 10.5 Hz, 1H), 4.80 (d, J = 15.5 Hz, 2H), 4.20 (dd, J = 11.6, 4.4 |
| | Hz, 1H), 3.67 (t, J = 11.1 Hz, 1H), 3.49 (d, J = 9.6 Hz, 1H), 2.36 (d, J = |
| | 13.6 Hz, 1H), 2.06 (d, J = 13.0 Hz, 1H), 1.85 (dd, J = 24.0, 13.4 Hz, |
| | 2H). (3 exchangeable protons are not seen) |
| 273 | MS m/z 431.3 [M + H]⁺; ¹HNMR(400 MHz, METHANOL-d₄) δ: 8.21 |
| | (dd, J = 60.9, 56.6 Hz, 1H), 7.49-7.45 (m, 1H), 6.74 (s, 1H), 6.38 (dt, J = |
| | 7.0, 2.2 Hz, 2H), 4.69 (d, J = 10.1 Hz, 1H), 4.59 (s, 2H), 4.13 (dd, J = |
| | 11.4, 4.5 Hz, 1H), 3.59 (dd, J = 17.8, 6.1 Hz, 1H), 3.05-2.92 (m, 1H), |
| | 2.21 (d, J = 13.3 Hz, 1H), 2.01-1.87 (m, 1H), 1.78 (d, J = 14.4 Hz, |
| | 1H), 1.56 (dt, J = 24.6, 6.9 Hz, 1H). (3 exchangeable protons are not |
| | seen) |
| 279 | MS m/z 427.95 [M + H]⁺; ¹H NMR (METHANOL-d₄) δ: 7.11-7.28 (m, |
| | 2H), 6.95-7.02 (m, 1H), 6.85-6.90 (m, 1H), 6.47 (s, 1H), 4.66 (s, 2H), |
| | 3.32 (br s, 1H), 3.22-3.25 (m, 1H), 2.06 (br s, 2H), 1.71-1.89 (m, 2H), |
| | 1.28-1.61 (m, 4H). (3 exchangeable protons are not seen) |
| 280 | MS m/z 386.3 [M + H]⁺; ¹HNMR(400 MHz, METHANOL-d₄) δ: 7.37- |
| | 7.21 (m, 5H), 6.31 (s, 1H), 4.56 (s, 2H), 2.97 (t, J = 10.9 Hz, 1H), 2.86 |
| | (t, J = 10.3 Hz, 1H), 2.34 (d, J = 6.6 Hz, 3H), 2.07-1.79 (m, 4H), 1.61- |
| | 1.30 (m, 4H). (3 exchangeable protons are not seen) |
| 281 | MS m/z 491.0 [M − H]⁻; ¹H NMR (DMSO-d₆) δ: 8.35-7.98 (m, 1H), |
| | 7.36 (dd, J = 5.0, 1.3 Hz, 1H), 7.10-7.02 (m, 1H), 6.94 (dd, J = 5.1, 3.4 |
| | Hz, 1H), 6.62 (s, 1H), 6.06 (s, 1H), 4.78 (d, J = 5.4 Hz, 2H), 4.42 (d, J = |
| | 10.0 Hz, 1H), 3.94 (d, J = 12.2 Hz, 1H), 3.39 (t, J = 11.5 Hz, 1H), 2.83 |
| | (s, 1H), 1.98 (d, J = 12.6 Hz, 1H), 1.86 (d, J = 13.5 Hz, 1H), 1.56 (d, J = |
| | 13.5 Hz, 1H), 1.33 (q, J = 11.2 Hz, 1H). (2, exchangeable protons not |
| | observed). |
| 283 | MS m/z 459.9 [M + H]⁺; ¹HNMR(400 MHz, METHANOL-d₄) δ: 6.60 (s, |
| | 1H), 4.10-3.94 (m, 2H), 3.26-3.10 (m, 1H), 3.06-2.94 (m, 1H), 2.15- |
| | 1.97 (m, 2H), 1.96-1.75 (m, 5H), 1.59-1.30 (m, 4H). (3 exchangeable |
| | protons are not seen) |
| 285 | MS m/z 460.8 [M + H]⁺; ¹HNMR(400 MHz, METHANOL-d₄) δ: 7.78 |
| | (d, J = 3.3 Hz, 1H), 7.54 (d, J = 3.3 Hz, 1H), 6.55 (s, 1H), 4.89 (d, J = |
| | 8.1 Hz, 2H), 4.04 (td, J = 11.8, 3.9 Hz, 2H), 3.56 (td, J = 11.7, 2.3 Hz, |
| | 1H), 3.40 (td, J = 11.5, 4.3 Hz, 1H), 3.25 (t, J = 10.7 Hz, 1H), 3.08 (td, J = |
| | 10.2, 4.5 Hz, 1H), 2.02-1.84 (m, 2H). (3 exchangeable protons are |
| | not seen) |
| 286 | MS m/z 460.0 [M + H]⁺; ¹HNMR(400 MHz, METHANOL-d₄) δ: 6.60 (s, |
| | 1H), 4.10-3.94 (m, 2H), 3.26-3.10 (m, 1H), 3.06-2.94 (m, 1H), 2.15- |
| | 1.97 (m, 2H), 1.96-1.75 (m, 5H), 1.59-1.30 (m, 4H). (3 exchangeable |
| | protons are not seen) |
| 291 | MS m/z 448.1 [M + H]⁺; ¹HNMR(400 MHz, METHANOL-d₄) δ: 7.32 |
| | (d, J = 5.1 Hz, 1H), 7.11 (s, 1H), 7.04-6.96 (m, 1H), 6.62 (s, 1H), 4.79 |
| | (s, 2H), 3.75 (dd, J = 27.8, 10.0 Hz, 1H), 3.16 (s, 1H), 2.24 (s, 1H), 2.04 |
| | (dd, J = 39.0, 27.5 Hz, 3H), 1.73 (d, J = 14.7 Hz, 1H), 1.51 (d, J = 11.6 |
| | Hz, 1H). (3 exchangeable protons are not seen) |
| 295 | MS m/z [M + H]⁺ 497.8; ¹H NMR (METHANOL-d₄) δ: 8.45 (s, 1H), |
| | 7.28-7.21 (m, 4H), 7.17 (d, J = 6.7 Hz, 1H), 6.31 (s, 1H), 4.48 (s, 2H), |
| | 3.29-3.23 (m, 2H), 2.09-1.91 (m, 2H), 1.88-1.72 (m, 2H), 1.55 (s, |
| | 1H), 1.40 (s, 3H). Formic acid salt, (3, exchangeable protons not |
| | observed). |
| 296 | MS m/z 395.9 [M + H]⁺; ¹HNMR(400 MHz, METHANOL-d₄) δ: 7.45 (s, |
| | 1H), 6.65 (s, 1H), 6.34 (dd, J = 12.6, 2.3 Hz, 2H), 4.54 (s, 2H), 3.19 (t, J = |
| | 9.4 Hz, 1H), 2.99 (s, 1H), 2.05 (dd, J = 27.2, 12.7 Hz, 2H), 1.88 (s, |
| | 2H), 1.67-1.57 (m, 1H), 1.52-1.38 (m, 3H). (3 exchangeable protons |
| | are not seen) |
| 301 | MS m/z 414.2 [M + H]⁺; ¹HNMR(400 MHz, METHANOL-d₄) δ: 6.63- |
| | 6.60 (m, 1H), 4.06 (d, J = 2.3 Hz, 2H), 3.17 (t, J = 10.9 Hz, 1H), 2.93 (t, |
| | J = 10.4 Hz, 1H), 2.03 (dd, J = 24.3, 12.8 Hz, 2H), 1.93-1.74 (m, 4H), |
| | 1.64-1.26 (m, 5H). (3 exchangeable protons are not seen) |
| 304 | MS m/z 521.9 [M + H]⁺; ¹HNMR(400 MHz, METHANOL-d₄) δ: 8.00 (t, |
| | J = 57.8 Hz, 1H), 7.43 (s, 1H), 6.35 (d, J = 3.0 Hz, 2H), 4.80 (s, 2H), |
| | 3.89 (s, 1H), 2.84 (s, 1H), 2.33 (s, 1H), 2.10 (s, 1H), 1.83 (s, 3H), 1.57- |
| | 1.27 (m, 3H). (3 exchangeable protons are not seen) |
| 305 | MS m/z 448.0 [M + H]⁺; ¹HNMR(400 MHz, METHANOL-d₄) δ: 8.29- |
| | 7.96 (m, 1H), 7.29 (d, J = 5.0 Hz, 1H), 7.12 (d, J = 3.3 Hz, 1H), 6.95 |
| | (dd, J = 5.0, 3.6 Hz, 1H), 5.06-4.92 (m, 3H), 4.20 (dd, J = 11.5, 4.3 |
| | Hz, 1H), 3.67 (t, J = 11.0 Hz, 1H), 3.53 (td, J = 11.1, 4.2 Hz, 1H), 2.36 |
| | (d, J = 9.9 Hz, 1H), 2.15-1.96 (m, 1H), 1.95-1.76 (m, 2H). (3 |
| | exchangeable protons are not seen) |
| 308 | MS m/z 368.1[M + H]⁺; ¹HNMR(400 MHz, METHANOL-d₄) δ: 6.63 (s, |
| | 1H), 4.07 (d, J = 2.2 Hz, 2H), 3.20-3.09 (m, 1H), 2.91 (s, 1H), 2.03 |
| | (dd, J = 25.7, 11.3 Hz, 2H), 1.89-1.77 (m, 5H), 1.60-1.30 (m, 4H). (3 |
| | exchangeable protons are not seen) |
| 321 | MS m/z 388.2 [M + H]⁺; ¹HNMR(400 MHz, METHANOL-d₄) δ: 7.34 |
| | (d, J = 6.8 Hz, 5H), 6.34 (s, 1H), 4.56 (s, 2H), 4.09 (d, J = 8.3 Hz, 1H), |
| | 3.96 (dd, J = 11.6, 4.5 Hz, 1H), 3.61 (t, J = 11.5 Hz, 1H), 3.49 (s, 2H), |
| | 2.38 (d, J = 4.2 Hz, 3H), 2.03 (dd, J = 13.3, 4.2 Hz, 1H), 1.70 (d, J = |
| | 16.3 Hz, 21H). (3 exchangeable protons are not seen) |
| 334 | MS m/z 431.6 [M + H]⁺; ¹H NMR (METHANOL-d₄) δ: 7.68-8.04 (m, |
| | 1H), 7.34 (s, 1H), 6.26 (s, 2H), 4.71 (d, J = 3.0 Hz, 2H), 3.61-3.76 (m, |
| | 1H), 2.86-3.01 (m, 1H), 1.96-2.09 (m, 2H), 1.79 (br s, 3H), 1.20-1.52 |
| | (m, 3H) (3 exchangeable protons are not seen) |
| 337 | MS m/z 415.0 [M + H]⁺; ¹HNMR(400 MHz, METHANOL-d₄) δ: 7.78 |
| | (d, J = 3.3 Hz, 1H), 7.54 (d, J = 3.3 Hz, 1H), 6.57 (s, 1H), 4.91 (s, 2H), |
| | 4.05 (s, 2H), 3.57 (s, 1H), 3.48 (s, 2H), 3.13 (s, 1H), 1.95 (s, 2H). (3 |
| | exchangeable protons are not seen) |
| 343 | MS m/z 415.8 [M + H]⁺; ¹HNMR(400 MHz, METHANOL-d₄) δ: 6.64 |
| | (d, J = 2.8 Hz, 1H), 4.71-4.64 (m, 1H), 4.06 (t, J = 7.8 Hz, 3H), 3.60 |
| | (dt, J = 11.8, 6.0 Hz, 1H), 2.92-2.77 (m, 1H), 2.15 (d, J = 10.0 Hz, |
| | 1H), 1.85-1.75 (m, 5H), 1.66-1.51 (m, 1H). (3 exchangeable protons |
| | are not seen) |
| 346 | MS m/z 448.0 [M + H]⁺; ¹HNMR(400 MHz, METHANOL-d₄) δ: 8.29- |
| | 7.96 (m, 1H), 7.29 (d, J = 5.0 Hz, 1H), 7.12 (d, J = 3.3 Hz, 1H), 6.95 |
| | (dd, J = 5.0, 3.6 Hz, 1H), 5.06-4.92 (m, 3H), 4.20 (dd, J = 11.5, 4.3 |
| | Hz, 1H), 3.67 (t, J = 11.0 Hz, 1H), 3.53 (td, J = 11.1, 4.2 Hz, 1H), 2.36 |
| | (d, J = 9.9 Hz, 1H), 2.15-1.96 (m, 1H), 1.95-1.76 (m, 2H). (3 |
| | exchangeable protons are not seen) |
| 351 | MS m/z 457.2 [M + H]⁺; ¹HNMR(400 MHz, METHANOL-d₄) δ: 7.30 |
| | (d, J = 5.1 Hz, 1H), 7.10 (d, J = 3.3 Hz, 1H), 6.98 (dd, J = 5.0, 3.5 Hz, |
| | 1H), 6.43 (d, J = 1.8 Hz, 1H), 4.68 (s, 2H), 4.10-3.98 (m, 5H), 3.78 |
| | (brs, 1H), 3.52 (t, J = 11.9 Hz, 1H), 3.18 (t, J = 10.6 Hz, 1H), 3.07 (brs, |
| | 1H), 2.45 (d, J = 12.5 Hz, 1H), 1.73 (d, J = 12.9 Hz, 1H). (3 |
| | exchangeable protons are not seen) |
| 356 | MS m/z 444.0 [M − H]⁻; ¹H NMR (DMSO-d₆) δ: 7.97 (br t, J = 5.8 Hz, |
| | 1H), 7.34 (d, J = 5.0 Hz, 1H), 7.04 (br s, 1H), 6.92 (t, J = 4.1 Hz, 1H), 6.56 |
| | (s, 1H), 4.66 (br d, J = 5.5 Hz, 2H), 2.98-3.44 (m, 4H), 1.92 (br s, 2H), |
| | 1.61-1.73 (m, 2H), 1.04-1.43 (m, 4H) |
| 360 | MS m/z 408.0 [M + H]⁺; ¹H NMR (METHANOL-d₄) δ: 7.16-7.23 (m, |
| | 1H), 6.94-7.00 (m, 1H), 6.84-6.91 (m, 1H), 6.45 (s, 1H), 4.79 (s, 2H), |
| | 4.68-4.72 (m, 1H), 4.66 (s, 2H), 3.24-3.29 (m, 1H), 2.00-2.12 (m, 2H), |
| | 1.75-1.92 (m, 2H), 1.56-1.69 (m, 1H), 1.36-1.51 (m, 3H) (4 |
| | exchangeable protons are not seen) |
| 361 | MS m/z 440.0 [M + H]⁺; ¹H NMR (METHANOL-d₄) δ: 8.55 (s, 1H), |
| | 6.46 (s, 1H), 5.60-5.58 (m, 1H), 4.02 (s, 2H), 3.26 (dd, J = 11.8, 3.4 |
| | Hz, 1H), 3.16-3.11 (m, 1H), 2.38-2.31 (m, 4H), 2.14-2.03 (m, 2H), |
| | 1.97-1.86 (m, 4H), 1.69-1.60 (m, 1H), 1.54-1.41 (m, 3H). Formic |
| | acid salt (3 Exchangeable protons not observed). |
| 363 | MS m/z 348.0 [M + H]⁺; ¹HNMR(400 MHz, METHANOL-d₄) δ: 6.55 (s, |
| | 1H), 4.07-4.01 (m, 2H), 3.02-2.78 (m, 2H), 2.36 (s, 3H), 2.10-2.00 (m, |
| | 1H), 2.09-1.86 (m, 3H), 1.85-1.75 (m, 3H), 1.62-1.34 (m, 4H). (3 |
| | exchangeable protons are not seen) |
| 364 | MS m/z 455.0 [M + H]⁺; ¹HNMR(400 MHz, METHANOL-d₄) δ: 8.49 |
| | (d, J = 6.1 Hz, 2H), 7.42 (d, J = 5.9 Hz, 2H), 6.38 (s, 1H), 4.67 (s, 2H), |
| | 4.16-3.97 (m, 2H), 3.56 (dd, J = 11.8, 9.4 Hz, 1H), 3.41 (td, J = 11.2, |
| | 4.3 Hz, 1H), 3.25 (t, J = 10.7 Hz, 1H), 3.08 (dt, J = 10.1, 5.0 Hz, 1H), |
| | 2.04-1.82 (m, 2H). (3 exchangeable protons are not seen) |
| 368 | MS m/z 406.2 [M + H]⁺; ¹HNMR(400 MHz, METHANOL-d₄) δ: 7.37- |
| | 7.31 (m, 4H), 7.26 (d, J = 6.5 Hz, 1H), 6.40 (s, 1H), 4.57 (s, 2H), 3.16 |
| | (d, J = 2.1 Hz, 1H), 2.93 (s, 1H), 1.99 (s, 2H), 1.86 (s, 2H), 1.49 (dd, J = |
| | 37.7, 28.3 Hz, 4H). (3 exchangeable protons are not seen) |
| 384 | MS m/z 457.1[M + H]⁺; ¹HNMR (400 MHz, METHANOL-d₄) δ: 7.30 |
| | (d, J = 5.1 Hz, 1H), 7.10 (d, J = 3.3 Hz, 1H), 6.98 (dd, J = 5.0, 3.5 Hz, |
| | 1H), 6.43 (d, J = 1.8 Hz, 1H), 4.68 (s, 2H), 4.10-3.98 (m, 5H), 3.78 |
| | (brs, 1H), 3.52 (t, J = 11.9 Hz, 1H), 3.18 (t, J = 10.6 Hz, 1H), 3.07 (brs, |
| | 1H), 2.45 (d, J = 12.5 Hz, 1H), 1.73 (d, J = 12.9 Hz, 1H). (3 |
| | exchangeable protons are not seen) |
| 389 | MS m/z 398.0 [M + H]⁺; ¹HNMR(400 MHz, METHANOL-d₄) δ: 7.35 (s, |
| | 1H), 6.62 (s, 1H), 6.30-6.23 (m, 2H), 4.46 (s, 2H), 4.05 (d, J = 8.2 Hz, |
| | 1H), 3.98 (d, J = 7.5 Hz, 1H), 3.56 (t, J = 10.9 Hz, 4H), 2.05 (d, J = 10.5 |
| | Hz, 1H), 1.79 (s, 1H). (3 exchangeable protons are not seen) |
| 391 | MS m/z 521.9 [M + H]⁺; ¹HNMR(400 MHz, METHANOL-d₄) δ: 8.00 (t, |
| | J = 57.7 Hz, 1H), 7.43 (s, 1H), 6.64-6.18 (m, 2H), 4.80 (s, 2H), 3.89 |
| | (s, 1H), 2.85 (s, 1H), 2.33 (s, 1H), 2.07 (s, 1H), 1.84 (s, 3H), 1.59-1.27 |
| | (m, 3H). (3 exchangeable protons are not seen) |
| 392 | MS m/z 431.9 [M + H]⁺; ¹H NMR (METHANOL-d₄) δ: 8.05-8.40 (m, |
| | 1H), 7.46 (s, 1H), 6.38 (s, 2H), 4.85 (br d, J = 2.8 Hz, 2H), 4.72 (br s, |
| | 1H), 4.10-4.21 (m, 1H), 3.55-3.66 (m, 1H), 2.95-3.09 (m, 1H), 2.19- |
| | 2.31 (m, 1H), 1.90-2.06 (m, 1H), 1.76-1.86 (m, 1H), 1.53-1.65 (m, 1H) |
| | (3 exchangeable protons are not seen) |
| 398 | MS m/z 416.1 [M + H]⁺; ¹HNMR(400 MHz, METHANOL-d₄) δ: 6.63 (s, |
| | 1H), 4.08-3.98 (m, 4H), 3.55 (d, J = 2.3 Hz, 1H), 3.43-3.34 (m, 1H), |
| | 3.24 (t, J = 10.7 Hz, 1H), 3.07 (dd, J = 10.3, 4.6 Hz, 1H), 2.03-1.82 |
| | (m, 2H), 1.80 (t, J = 2.0 Hz, 3H). (3 exchangeable protons are not seen) |
| 400 | MS m/z 410.0 [M + H]⁺; ¹H NMR (METHANOL-d₄) δ: 8.44 (s, 1H), |
| | 7.19 (d, J = 5.0 Hz, 1H), 6.98 (d, J = 3.3 Hz, 1H), 6.85-6.91 (m, 1H), 6.46 |
| | (s, 1H), 5.46-5.67 (m, 2H), 4.66 (s, 2H), 3.13-3.19 (m, 1H), 2.97-3.08 |
| | (m, 1H), 1.96-2.09 (m, 2H), 1.73-1.89 (m, 2H), 1.52-1.69 (m, 1H), 1.33- |
| | 1.50 (m, 3H). (3 exchangeable protons are not seen) |
| 401 | MS m/z 376.9 [M + H]⁺; ¹HNMR(400 MHz, METHANOL-d₄) δ: 7.44 |
| | (d, J = 1.0 Hz, 1H), 6.54 (s, 1H), 6.35 (dd, J = 3.2, 1.9 Hz, 1H), 6.30 (d, |
| | J = 2.6 Hz, 1H), 4.52 (s, 2H), 3.01-2.92 (m, 1H), 2.84 (td, J = 10.5, 3.8 |
| | Hz, 1H), 2.34 (s, 3H), 2.04 (dd, J = 13.8, 4.4 Hz, 1H), 1.99-1.91 (m, |
| | 1H), 1.85 (t, J = 10.7 Hz, 2H), 1.57-1.32 (m, 4H). (3 exchangeable |
| | protons are not seen) |
| 405 | MS m/z 414.0 [M + H]⁺; ¹HNMR(400 MHz, METHANOL-d₄) δ: 7.30 |
| | (dd, J = 5.1, 1.1 Hz, 1H), 7.08 (d, J = 3.3 Hz, 1H), 6.98 (dd, J = 5.1, 3.5 |
| | Hz, 1H), 6.62 (d, J = 1.9 Hz, 1H), 4.76 (s, 2H), 4.14-4.00 (m, 2H), |
| | 3.67-3.50 (m, 3H), 3.41 (s, 1H), 2.07 (d, J = 13.0 Hz, 1H), 1.76 (d, J = |
| | 11.2 Hz, 1H). (3 exchangeable protons are not seen) |
| 406 | MS m/z 408.1 [M + H]⁺; ¹HNMR(400 MHz, METHANOL-d₄) δ: 7.39- |
| | 7.31 (m, 4H), 7.27 (d, J = 6.3 Hz, 1H), 6.42 (s, 1H), 4.58 (s, 2H), 4.07 |
| | (d, J = 7.6 Hz, 1H), 3.99 (dd, J = 11.0, 4.1 Hz, 1H), 3.60 (t, J = 11.1 Hz, |
| | 1H), 3.48 (t, J = 10.8 Hz, 1H), 3.40 (dd, J = 9.9, 4.1 Hz, 1H), 3.15 (td, J = |
| | 10.4, 4.0 Hz, 1H), 2.00 (d, J = 9.4 Hz, 1H), 1.73-1.58 (m, 1H). (3 |
| | exchangeable protons are not seen) |
| 407 | MS m/z 415.0 [M + H]⁺; ¹HNMR(400 MHz, METHANOL-d₄) δ: 7.78 |
| | (d, J = 3.3 Hz, 1H), 7.54 (d, J = 3.3 Hz, 1H), 6.57 (s, 1H), 4.91 (s, 2H), |
| | 4.06 (dd, J = 19.4, 8.1 Hz, 2H), 3.55 (d, J = 8.3 Hz, 1H), 3.45 (d, J = |
| | 22.3 Hz, 2H), 3.13 (s, 1H), 1.96 (d, J = 11.3 Hz, 2H). (3 exchangeable |
| | protons are not seen) |
| 408 | MS m/z 507.0 [M + H]⁺; ¹HNMR(400 MHz, METHANOL-d₄) δ: 7.79 (s, |
| | 1H), 7.55 (s, 1H), 6.54 (s, 1H), 4.91 (s, 2H), 4.08 (d, J = 10.9 Hz, 2H), |
| | 3.58 (t, J = 11.9 Hz, 1H), 3.43 (d, J = 8.8 Hz, 1H), 3.29 (s, 2H), 1.98 |
| | (dd, J = 31.8, 14.2 Hz, 2H). (3 exchangeable protons are not seen) |
| 409 | MS m/z 448.1 [M + H]⁺; ¹HNMR(400 MHz, METHANOL-d₄) δ: 7.32 |
| | (d, J = 5.1 Hz, 1H), 7.11 (s, 1H), 7.04-6.96 (m, 1H), 6.62 (s, 1H), 4.79 |
| | (s, 2H), 3.75 (dd, J = 27.8, 10.0 Hz, 1H), 3.16 (s, 1H), 2.24 (s, 1H), 2.04 |
| | (dd, J = 39.0, 27.5 Hz, 3H), 1.73 (d, J = 14.7 Hz, 1H), 1.51 (d, J = 11.6 |
| | Hz, 1H). (3 exchangeable protons are not seen) |
| 412 | MS m/z 477.0 [M + H]⁺; ¹HNMR(400 MHz, METHANOL-d₄) δ: 7.45- |
| | 7.41 (m, 1H), 6.37-6.32 (m, 2H), 4.82-4.70 (m, 2H), 3.78 (dd, J = |
| | 28.1, 12.0 Hz, 1H), 3.13 (s, 1H), 2.21 (s, 1H), 2.12-1.91 (m, 3H), 1.71 |
| | (dd, J = 16.7, 11.9 Hz, 1H), 1.54-1.43 (m, 1H). (3 exchangeable |
| | protons are not seen) |
| 416 | MS m/z 540.0 [M + H]⁺; ¹HNMR(400 MHz, METHANOL-d₄) δ: 8.24 |
| | (dd, J = 60.5, 56.5 Hz, 1H), 7.28 (dd, J = 5.1, 1.1 Hz, 1H), 7.11 (d, J = |
| | 2.7 Hz, 1H), 6.95 (dd, J = 5.1, 3.5 Hz, 1H), 5.00 (t, J = 9.7 Hz, 2H), 4.71 |
| | (d, J = 10.1 Hz, 1H), 4.13 (d, J = 7.2 Hz, 1H), 3.56 (t, J = 11.0 Hz, 1H), |
| | 2.92 (d, J = 10.5 Hz, 1H), 2.21 (d, J = 12.6 Hz, 1H), 2.03-1.84 (m, |
| | 1H), 1.77 (d, J = 13.5 Hz, 1H), 1.54 (dd, J = 24.1, 8.9 Hz, 1H. (3 |
| | exchangeable protons are not seen) |
| 418 | MS m/z 440.0 [M + H]⁺; ¹H NMR (METHANOL-d₄) δ: 7.87 (s, 1H), |
| | 7.11-7.34 (m, 5H), 4.72 (s, 2H), 3.83-3.98 (m, 1H), 2.99-3.12 (m, 1H), |
| | 1.95-2.14 (m, 2H), 1.75-1.91 (m, 3H), 1.31-1.50 (m, 3H) (3 |
| | exchangeable protons are not seen) |
| 421 | MS m/z 445.1 [M + H]⁺; ¹H NMR (METHANOL-d₄) δ: 8.44 (s, 1H), |
| | 8.04-7.69 (m, 1H), 7.24 (d, J = 5.1 Hz, 1H), 7.02 (d, J = 3.4 Hz, 1H), |
| | 6.90 (dd, J = 5.2, 3.5 Hz, 1H), 6.61 (s, 1H), 4.67 (s, 2H), 3.74 (s, 1H), |
| | 2.95 (s, 1H), 2.05 (d, J = 12.7 Hz, 2H), 1.89-1.71 (m, 3H), 1.52-1.24 |
| | (m, 3H). Formic acid salt, (3, exchangeable protons not observed). |
| 422 | MS m/z 523.9 [M + H]⁺; ¹HNMR(400 MHz, METHANOL-d₄) δ: 8.26 |
| | (dd, J = 60.5, 56.4 Hz, 1H), 7.44 (s, 1H), 6.35 (d, J = 3.1 Hz, 2H), 4.80 |
| | (t, J = 9.3 Hz, 2H), 4.73 (d, J = 10.1 Hz, 1H), 4.14 (dd, J = 11.6, 4.4 Hz, |
| | 1H), 3.57 (t, J = 11.0 Hz, 1H), 2.97 (t, J = 8.6 Hz, 1H), 2.22 (d, J = 10.2 |
| | Hz, 1H), 2.04-1.88 (m, 1H), 1.78 (d, J = 13.6 Hz, 1H), 1.65-1.48 (m, |
| | 1H). (3 exchangeable protons are not seen) |
| 423 | MS m/z 448.0 [M + H]⁺; ¹HNMR(400 MHz, METHANOL-d₄) δ: 8.29- |
| | 7.96 (m, 1H), 7.29 (d, J = 5.0 Hz, 1H), 7.12 (d, J = 3.3 Hz, 1H), 6.95 |
| | (dd, J = 5.0, 3.6 Hz, 1H), 5.06-4.92 (m, 3H), 4.20 (dd, J = 11.5, 4.3 |
| | Hz, 1H), 3.67 (t, J = 11.0 Hz, 1H), 3.53 (td, J = 11.1, 4.2 Hz, 1H), 2.36 |
| | (d, J = 9.9 Hz, 1H), 2.15-1.96 (m, 1H), 1.95-1.76 (m, 2H). (3 |
| | exchangeable protons are not seen) |
| 425 | MS m/z 376.1 [M + H]⁺; ¹H NMR (METHANOL-d₄) δ: 7.20 (dd, J = 5.1, |
| | 1.2 Hz, 1H), 7.17 (s, 1H), 7.02-6.96 (m, 1H), 6.88 (dd, J = 5.1, 3.5 Hz, |
| | 1H), 6.49 (s, 1H), 5.79 (d, J = 10.5 Hz, 1H), 5.67 (d, J = 10.5 Hz, 1H), |
| | 4.67 (s, 2H), 3.60-3.49 (m, 1H), 3.33 (ddd, J = 10.1, 10.1, 5.5 Hz, 1H), |
| | 2.62-2.42 (m, 3H), 2.25-2.14 (m, 1H). (3 Exchangeable protons not |
| | observed). |
| 426 | MS m/z 476.8 [M + H]⁺; ¹HNMR(400 MHz, METHANOL-d₄) δ: 8.24 |
| | (dd, J = 61.1, 56.6 Hz, 1H), 7.48 (s, 1H), 6.72 (d, J = 18.4 Hz, 1H), 6.40- |
| | 6.27 (m, 2H), 4.73 (t, J = 11.7 Hz, 1H), 4.59 (s, 2H), 4.18-4.05 (m, |
| | 1H), 3.58 (t, J = 12.0 Hz, 1H), 2.97 (s, 1H), 2.22 (d, J = 9.6 Hz, 1H), |
| | 1.95 (d, J = 13.6 Hz, 1H), 1.79 (d, J = 13.0 Hz, 1H), 1.63-1.48 (m, |
| | 1H) . . . (3 exchangeable protons are not seen) |
| 428 | MS m/z 414.1 [M + H]⁺; ¹HNMR(400 MHz, METHANOL-d₄) δ: 6.62 |
| | (d, J = 2.4 Hz, 1H), 4.06 (d, J = 2.2 Hz, 2H), 3.22-3.11 (m, 1H), 2.98- |
| | 2.88 (m, 1H), 2.03 (dd, J = 23.6, 12.6 Hz, 2H), 1.94-1.73 (m, 4H), 1.61- |
| | 1.27 (m, 5H). (3 exchangeable protons are not seen) |
| 432 | MS m/z 453.1 [M + H]⁺; ¹HNMR(400 MHz, METHANOL-d₄) δ: 8.51 |
| | (d, J = 5.9 Hz, 2H), 7.44 (d, J = 5.6 Hz, 2H), 6.38 (d, J = 7.0 Hz, 1H), |
| | 4.69 (s, 2H), 3.22 (dd, J = 15.5, 6.8 Hz, 1H), 2.98 (t, J = 10.0 Hz, 1H), |
| | 2.08 (dd, J = 20.6, 13.3 Hz, 2H), 1.90 (s, 2H), 1.67-1.35 (m, 4H). (3 |
| | exchangeable protons are not seen) |
| 442 | MS m/z 370.1 [M + H]⁺; ¹H NMR (METHANOL-d₄) δ: 6.51 (s, 1H), |
| | 5.80-5.67 (m, 1H), 5.50 (dtd, J = 10.7, 6.4, 1.8 Hz, 1H), 4.02 (d, J = |
| | 6.4 Hz, 2H), 3.46-3.35 (m, 2H), 2.25-2.18 (m, 1H), 2.15-2.08 (m, |
| | 1H), 2.02-1.88 (m, 2H), 1.80 (d, J = 6.9, 3H), 1.78-1.70 (m, 1H), |
| | 1.67-1.42 (m, 3H). (3 Exchangeable protons not observed). |

### Example 3

### Preparation of Compound 129 and Compound 138

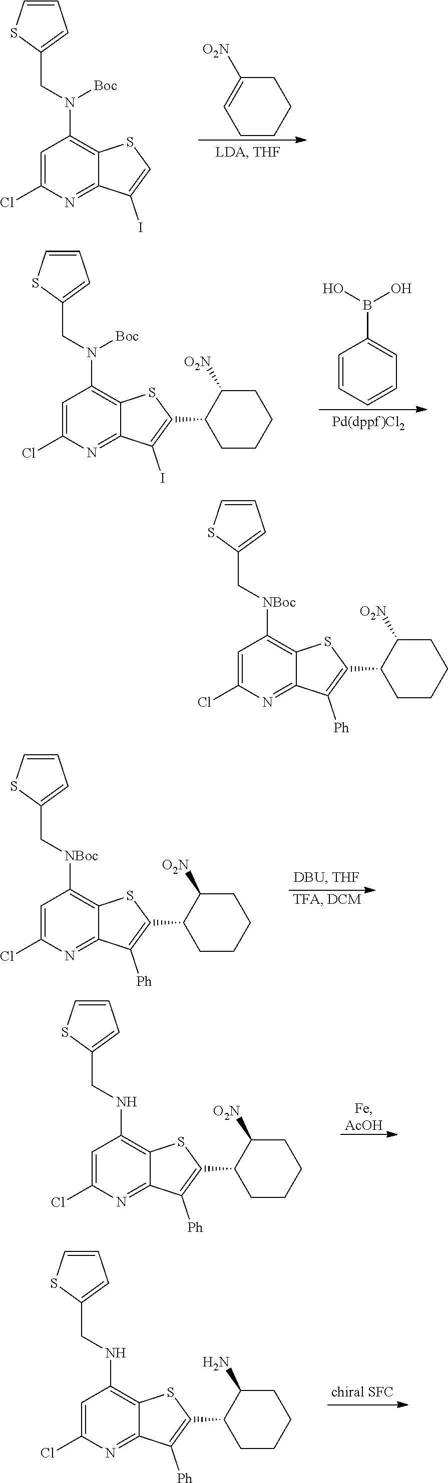

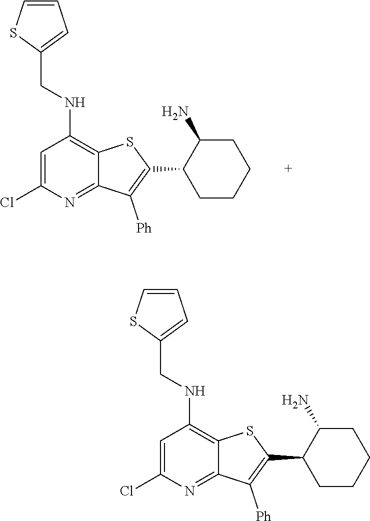 [SMILES: N[C@H]1CCCC[C@@H]1c1sc2c(NCc3cccs3)cc(Cl)nc2c1-c1ccccc1 ; N[C@@H]1CCCC[C@H]1c1sc2c(NCc3cccs3)cc(Cl)nc2c1-c1ccccc1]

### Step 1: tert-Butyl N-[5-chloro-3-iodo-2-[(1S,2R)-2-nitrocyclohexyl]thieno[3,2-b]pyridin-7-yl]-N-(2-thienylmethyl)carbamate

To a solution of tert-butyl N-(5-chloro-3-iodo-thieno[3,2-b]pyridin-7-yl)-N-(2-thienylmethyl)carbamate (1, 1 g, 2.0 mmol) in THF (10 mL) was added lithium diisopropylamide (2M) in THF/heptane/ethylbenzene (1.28 mL) at −78° C. and stirred for 1 h, then added 1-nitrocyclohexene (2, 497.5 mg, 4 mmol) at −78° C. The mixture was stirred at −78° C. for 3 h. The mixture was quenched with saturated ammonium chloride solution and extracted with EtOAc. The combined organic layers dried over Na₂SO₄, filtered, and concentrated in vacuo. The crude residue was purified by flash column eluting with (EtOAc/PE˜5.1%) to give tert-butyl N-[5-chloro-3-iodo-2-[(1S,2R)-2-nitrocyclohexyl]thieno[3,2-b]pyridin-7-yl]-N-(2-thienylmethyl)carbamate (1.1 g, 88% Yield) as a white solid. MS m/z 633.8 [M+H]⁺.

### Step 2: tert-Butyl N-[5-chloro-2-[(1S,2R)-2-nitrocyclohexyl]-3-phenyl-thieno[3,2-b]pyridin-7-yl]-N-(2-thienylmethyl)carbamate

To a solution of tert-butyl N-[5-chloro-3-iodo-2-[(1S,2R)-2-nitrocyclohexyl]thieno[3,2-b]pyridin-7-yl]-N-(2-thienylmethyl)carbamate (300 mg, 0.5 mmol) in 1,4-dioxane (6 mL) and H₂O (1.2 mL) was added K₂CO₃ (163.26 mg, 1.2 mmol) and phenylboronic acid (57.7 mg, 0.473 mmol) at 25° C. Then the mixture was stirred at 65° C. for 2.5 hours. The reaction mixture was poured into water and extracted with EtOAc, then washed with brine and dried over Na₂SO₄, filtered and concentrated to dryness to get crude product which was purified by flash column eluting with (EtOAc/PE˜15%) to give tert-butyl N-[5-chloro-2-[(1S,2R)-2-nitrocyclohexyl]-3-phenyl-thieno[3,2-b]pyridin-7-yl]-N-(2-thienylmethyl)carbamate (200 mg, 72% Yield) as a yellow oil. MS m/z 583.9 [M+H]⁺.

### Step 3: tert-Butyl N-[5-chloro-2-[(1S,2S)-2-nitrocyclohexyl]-3-phenyl-thieno[3,2-b]pyridin-7-yl]-N-(2-thienylmethyl)carbamate

To a solution of tert-butyl N-[5-chloro-2-[(1S,2R)-2-nitrocyclohexyl]-3-phenyl-thieno[3,2-b]pyridin-7-yl]-N-(2-thienylmethyl)carbamate (200 mg, 0.3 mmol) in THF (5 mL) was added diazabicycloundecane (52 mg, 0.3 mmol, 1.0 eq) at 0° C. The mixture was stirred at 0° C. for 5 h. The mixture was added H₂O and extracted with EtOAc. The combined organic layers were washed with brine, dried over Na₂SO₄, filtered, and concentrated in vacuo to give tert-butyl N-[5-chloro-2-[(1S,2S)-2-nitrocyclohexyl]-3-phenyl-thieno[3,2-b]pyridin-7-yl]-N-(2-thienylmethyl)carbamate (140 mg, 93% Yield) as a light yellow solid. ¹H NMR (CHLOROFORM-d) δ ppm 7.51 (t, J=7.3 Hz, 2H), 7.46 (d, J=7.2 Hz, 1H), 7.39 (d, J=7.0 Hz, 2H), 7.24 (s, 1H), 6.99 (s, 1H), 6.92-6.88 (m, 1H), 6.83 (d, J=3.4 Hz, 1H), 5.03 (s, 2H), 4.69 (td, J=11.4, 4.1 Hz, 1H), 3.81-3.71 (m, 1H), 2.37 (d, J=12.9 Hz, 1H), 2.10 (d, J=10.6 Hz, 1H), 1.98-1.77 (m, 4H), 1.61 (s, 2H), 1.47 (s, 9H).

### Step 4: 5-Chloro-2-[(1S,2S)-2-nitrocyclohexyl]-3-phenyl-N-(2-thienylmethyl)thieno[3,2-b]pyridin-7-amine

To a solution of tert-butyl N-[5-chloro-2-[(1S,2S)-2-nitrocyclohexyl]-3-phenyl-thieno[3,2-b]pyridin-7-yl]-N-(2-thienylmethyl)carbamate (140 mg, 0.2 mmol) in DCM (4 mL) was added TFA (4 mL). The mixture was stirred at rt for 1 h. The mixture was concentrated under vacuum to give the crude 5-chloro-2-[(1S,2S)-2-nitrocyclohexyl]-3-phenyl-N-(2-thienylmethyl)thieno[3,2-b]pyridin-7-amine (116 mg, 100% Yield) as a brown oil. MS m/z 484.0 [M+H]⁺.

### Step 5: 2-((1S,2S)-2-Aminocyclohexyl)-5-chloro-3-phenyl-N-(thiophen-2-ylmethyl)thieno[3,2-b]pyridin-7-amine and 2-((1R,2R)-2-aminocyclohexyl)-5-chloro-3-phenyl-N-(thiophen-2-ylmethyl)thieno [3,2-b]pyridin-7-amine

To a solution of 5-chloro-2-[(1S,2S)-2-nitrocyclohexyl]-3-phenyl-N-(2-thienylmethyl)thieno[3,2-b]pyridin-7-amine (116 mg, 0.2 mmol) in MeOH (8 mL) and AcOH (2 mL) was added Iron (269 mg, 4.8 mmol). The mixture was stirred at 70° C. for 16 h. The mixture was filtered with diatomaceous earth, then concentrated and purified by flash column eluting with (MeOH/DCM˜6.4%) to give 2-[(1S,2S)-2-aminocyclohexyl]-5-chloro-3-cyclopropyl-N-(2-thienylmethyl)thieno[3,2-b]pyridin-7-amine (8, 130 mg) as a yellow oil. The oil was purified by SFC (DAICELCHIRALPAK® OJ, Supercritical CO₂/MeOH(+0.1% 7.0 mol/l Ammonia in MeOH)/MeOH) to give 2-((1S,2S)-2-aminocyclohexyl)-5-chloro-3-phenyl-N-(thiophen-2-ylmethyl)thieno[3,2-b]pyridin-7-amine (Compound 129, 28 mg, 22% Yield) and 2-((1R,2R)-2-aminocyclohexyl)-5-chloro-3-phenyl-N-(thiophen-2-ylmethyl)thieno[3,2-b]pyridin-7-amine (Compound 138, 42.2 mg, 33% Yield) as a white solid.

Compound 129: MS m/z 454.0 [M+H]⁺; ¹H NMR (METHANOL-d₄) δ ppm 7.47 (dd, J=9.0, 5.7 Hz, 2H), 7.43-7.38 (m, 3H), 7.29 (dd, J=5.1, 1.2 Hz, 1H), 7.09-7.06 (m, 1H), 6.98 (dd, J=5.1, 3.5 Hz, 1H), 6.51 (s, 1H), 4.77 (s, 2H), 299-2.81 (m, 2H), 2.07-1.93 (m, 2H), 1.79 (s, 2H), 1.64 (d, J=11.3 Hz, 1H), 1.51-1.42 (m, 1H), 1.29 (m, 2H) (3 exchangeable protons are not seen).

Compound 138: MS m/z 454.0 [M+H]⁺; ¹H NMR (METHANOL-d₄) δ ppm 7.51 (t, J=7.2 Hz, 2H), 7.46-7.39 (m, 3H), 7.30 (dd, J=5.1, 1.2 Hz, 2H), 7.11-7.08 (m, 1H), 6.98 (dd, J=5.1, 3.5 Hz, 1H), 4.78 (s, 2H), 3.33 (s, 1H), 3.12 (d, J=3.5 Hz, 1H), 2.21 (s, 1H), 2.07 (s, 1H), 1.87 (d, J=10.7 Hz, 2H), 1.82-1.74 (m, 1H), 1.48 (d, J=11.4 Hz, 1H), 1.38-1.31 (m, 2H) (3 exchangeable protons are not seen).

The compounds below were prepared according to the procedure of Example 3 by substituting the appropriate starting materials, reagents, reaction conditions, and followed by chiral SFC resolution if needed.

| Compound | Spectral Data |
|---|---|
| 130 | MS m/z 418.2 [M + H]⁺; ¹HNMR(400 MHz, METHANOL-d₄) δ: 7.27 |
| | (d, J = 5.0 Hz, 1H), 7.05 (s, 1H), 6.96 (s, 1H), 6.44 (s, 1H), 4.72 (s, 2H), |
| | 3.25-3.20 (m, 1H), 2.89 (s, 1H), 2.03 (dd, J = 35.4, 11.6 Hz, 2H), 1.88 |
| | (d, J = 5.5 Hz, 3H), 1.58-1.31 (m, 4H), 1.04 (s, 2H), 0.97 (d, J = 8.2 |
| | Hz, 2H). (3 exchangeable protons are not seen) |
| 131 | MS m/z 440.0 [M + H]⁺; ¹HNMR(400 MHz, METHANOL-d₄) δ: 7.47 |
| | (d, J = 7.1 Hz, 2H), 7.41 (dd, J = 5.2, 2.4 Hz, 3H), 7.29 (dd, J = 5.1, 1.0 |
| | Hz, 1H), 7.08 (d, J = 2.6 Hz, 1H), 7.00-6.96 (m, 1H), 6.51 (s, 1H), |
| | 4.77 (s, 2H), 3.51-3.43 (m, 1H), 3.27 (s, 1H), 2.23 (t, J = 8.1 Hz, 1H), |
| | 2.15 (dd, J = 12.6, 5.3 Hz, 1H), 1.90-1.80 (m, 3H), 1.52 (d, J = 8.3 Hz, |
| | 1H). (3 exchangeable protons are not seen) |
| 135 | MS m/z 440.0 [M + H]⁺; ¹HNMR(400 MHz, METHANOL-d₄) δ: 7.46 |
| | (d, J = 6.6 Hz, 2H), 7.41 (d, J = 6.8 Hz, 3H), 7.29 (d, J = 4.0 Hz, 1H), |
| | 7.08 (d, J = 2.5 Hz, 1H), 6.98 (dd, J = 5.0, 3.5 Hz, 1H), 6.50 (s, 1H), |
| | 4.76 (s, 2H), 3.44 (s, 1H), 3.23 (d, J = 9.0 Hz, 1H), 2.23 (d, J = 8.1 Hz, |
| | 1H), 2.18-2.08 (m, 1H), 1.91-1.78 (m, 3H), 1.50 (dd, J = 15.6, 6.4 |
| | Hz, 1H). (3 exchangeable protons are not seen) |
| 137 | MS m/z 418.2 [M + H]⁺; ¹HNMR(400 MHz, METHANOL-d₄) δ: 7.27 (s, |
| | 1H), 7.05 (s, 1H), 6.96 (s, 1H), 6.49 (s, 1H), 4.73 (s, 2H), 3.53 (s, 1H), |
| | 2.21 (s, 1H), 2.10 (d, J = 12.2 Hz, 1H), 1.92 (dd, J = 31.7, 18.6 Hz, 4H), |
| | 1.64 (dd, J = 54.3, 11.9 Hz, 4H), 1.06 (s, 2H), 1.01 (d, J = 8.2 Hz, 2H). |
| | (3 exchangeable protons are not seen) |
| 141 | MS m/z 469.8 [M + H]⁺; ¹HNMR(400 MHz, METHANOL-d₄) δ: 7.48 (t, |
| | J = 7.3 Hz, 2H), 7.40 (dd, J = 8.7, 7.0 Hz, 3H), 7.29 (dd, J = 5.1, 1.1 Hz, |
| | 1H), 7.08 (d, J = 2.5 Hz, 1H), 6.98 (dd, J = 5.1, 3.5 Hz, 1H), 6.51 (s, |
| | 1H), 4.77 (s, 2H), 3.20 (s, 1H), 3.07 (dd, J = 12.2, 6.5 Hz, 1H), 1.97- |
| | 1.81 (m, 4H), 1.73-158 (m, 10H). (3 exchangeable protons are not seen) |
| 142 | MS m/z 469.8 [M + H]⁺; ¹HNMR(400 MHz, METHANOL-d₄) δ: 7.51 (t, |
| | J = 7.2 Hz, 2H), 7.45-7.38 (m, 3H), 7.30 (dd, J = 5.1, 1.2 Hz, 1H), 7.09 |
| | (d, J = 2.5 Hz, 1H), 6.98 (dd, J = 5.1, 3.5 Hz, 1H), 6.54 (s, 1H), 4.78 (s, |
| | 2H), 3.49-3.38 (m, 1H), 3.25-3.17 (m, 1H), 2.08-158 (m, 10H). (3 |
| | exchangeable protons are not seen) |
| 267 | MS m/z 402.1 [M + H]⁺; ¹H NMR (METHANOL-d₄) δ: 7.30 (dd, J = 5.1, |
| | 1.3 Hz, 1H), 7.08 (dd, J = 3.2, 1.3 Hz, 1H), 7.01-6.97 (m, 1H), 6.94 |
| | (dd, J = 16.8, 10.9 Hz, 1H), 6.58 (s, 1H), 6.42 (dd, J = 16.8, 2.1 Hz, |
| | 1H), 5.90 (d, J = 10.5 Hz, 1H), 5.78 (d, J = 10.5 Hz, 1H), 5.61 (dd, J = |
| | 10.9, 2.1 Hz, 1H), 4.76 (s, 2H), 3.79 (ddd, J = 9.9, 9.8, 5.8 Hz, 1H), 3.67 |
| | (ddd, J = 9.8, 9.7, 5.3 Hz, 1H), 2.70-2.56 (m, 3H), 2.38-2.27 (m, |
| | 1H). (3 Exchangeable protons not observed). |
| 350 | MS m/z 406.1 [M + H]⁺; ¹H NMR (METHANOL-d₄) δ: 8.55 (s, 0.4H, |
| | formic acid), 7.29 (dd, J = 5.1, 1.2 Hz, 1H), 7.07 (dt, J = 3.5, 1.2 Hz, |
| | 1H), 7.03-6.91 (m, 2H), 6.51 (s, 1H), 6.38 (dd, J = 17.7, 2.2 Hz, 1H), |
| | 5.53 (dd, J = 11.7, 2.2 Hz, 1H), 4.74 (s, 2H), 4.10-3.98 (m, 2H), 3.56 |
| | (td, J = 11.3, 4.2 Hz, 1H), 3.42-3.33 (m, 1H), 3.27-3.18 (m, 1H), |
| | 3.02 (td, J = 10.2, 4.6 Hz, 1H), 1.98-1.85 (m, 2H). (3 Exchangeable |
| | protons not observed). |
| 390 | MS m/z 391.1 [M + H]⁺; ¹H NMR (METHANOL-d₄) δ: 8.55 (s, 0.2H, |
| | formic acid), 7.43 (s, 1H), 6.94 (dd, J = 17.7, 11.9 Hz, 1H), 6.44 (dd, J = |
| | 17.7, 2.4 Hz, 1H), 6.39-6.30 (m, 2H), 5.55 (dd, J = 11.7, 2.1 Hz, 1H), |
| | 4.80-4.69 (m, 2H), 4.10-3.95 (m, 2H), 3.54 (td, J = 11.7, 2.6 Hz, |
| | 1H), 3.36 (td, J = 11.0, 4.5 Hz, 1H), 3.21 (t, J = 10.7 Hz, 1H), 2.99 (td, J = |
| | 10.2, 4.5 Hz, 1H), 1.96-1.81 (m, 2H). (3 Exchangeable protons not |
| | observed). |

### Example 4

### Preparation of Compound 103, Compound 104, Compound 105, and Compound 106

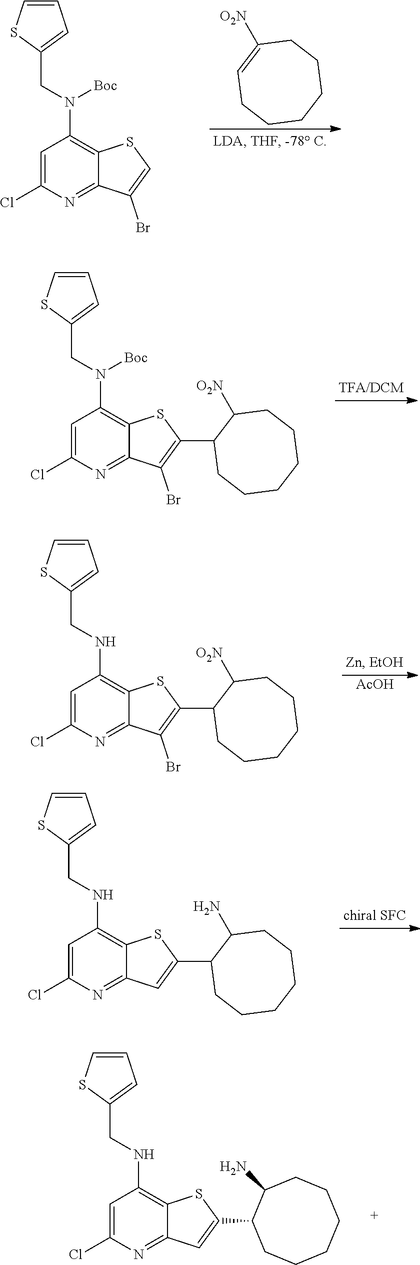

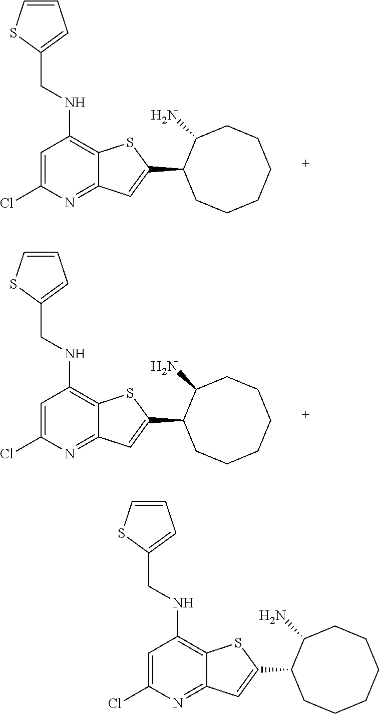 [SMILES: N[C@@H]1CCCCCC[C@H]1c1cc2nc(Cl)cc(NCc3cccs3)c2s1 ; N[C@H]1CCCCCC[C@H]1c1cc2nc(Cl)cc(NCc3cccs3)c2s1 ; N[C@@H]1CCCCCC[C@@H]1c1cc2nc(Cl)cc(NCc3cccs3)c2s1]

### Step 1. tert-Butyl (3-bromo-5-chloro-2-(2-nitrocyclooctyl)thieno[3,2-b]pyridin-7-yl)(thiophen-2-ylmethyl)carbamate

To a solution of 24-1 (1.0 g, 2.17 mmol) in THF (12 mL) was added 2M lithium diisopropylamide (1.63 mL) at −78° C. and stirred for 1 h, then added (E)-1-nitrocyclooct-1-ene (506.3 mg, 3.26 mmol) at −78° C. The mixture was stirred at −78° C. for 3 h. The reaction mixture was then quenched with saturated ammonium chloride solution and extracted with EtOAc. The combined organic layers were dried over Na₂SO₄, filtered, and concentrated in vacuo. The crude residue was purified by flash column eluting with (EtOAc/PE˜5.1%) to give 24-2 (550 mg, 41% Yield) as a white solid. ¹H NMR (CHLOROFORM-d) δ ppm 7.22 (dd, J=5.1, 1.1 Hz, 1H), 7.07 (s, 1H), 6.87 (dd, J=5.1, 3.5 Hz, 1H), 6.79 (d, J=2.9 Hz, 1H), 5.06-4.94 (m, 3H), 4.27 (dt, J=11.0, 3.8 Hz, 1H), 2.51-2.32 (m, 3H), 2.10-1.90 (m, 3H), 1.81-1.67 (m, 6H), 1.44 (s, 9H).

### Step 2. 3-Bromo-5-chloro-2-(2-nitrocyclooctyl)-N-(thiophen-2-ylmethyl)thieno[3,2-b]pyridin-7-amine

To a solution of tert-butyl (3-bromo-5-chloro-2-(2-nitrocyclooctyl)thieno[3,2-b]pyridin-7-yl)(thiophen-2-ylmethyl)carbamate (550 mg, 0.89 mmol) in DCM (8 mL) was added TFA (8 mL). The mixture was stirred at rt for 1 h. The mixture was concentrated under vacuum to give the crude 3-bromo-5-chloro-2-(2-nitrocyclooctyl)-N-(thiophen-2-ylmethyl)thieno[3,2-b]pyridin-7-amine (130 mg, 28% Yield) as a brown oil. MS m/z 516.0 [M+H]⁺.

### Step 3. 2-((1S,2S)-2-Aminocyclooctyl)-5-chloro-N-(thiophen-2-ylmethyl)thieno[3,2-b]pyridin-7-amine, 2-((1R,2R)-2-aminocyclooctyl)-5-chloro-N-(thiophen-2-ylmethyl)thieno[3,2-b]pyridin-7-amine, 2-((1R,2S)-2-aminocyclooctyl)-5-chloro-N-(thiophen-2-ylmethyl)thieno[3,2-b]pyridin-7-amine, and 2-((1S,2R)-2-aminocyclooctyl)-5-chloro-N-(thiophen-2-ylmethyl)thieno[3,2-b]pyridin-7-amine

To a solution of 24-3 (130 mg, 0.25 mmol) in MeOH (8 mL) and AcOH (2 mL) was added Iron (82.5 mg, 1.26 mmol). The mixture was stirred at 100° C. for 16 h. The reaction mixture was cooled to rt and filtered with diatomaceous earth, then concentrated and purified by flash column eluting with (MeOH/DCM˜6.4%) to give a white solid. The solid was purified by SFC (DAICELCHIRALPAK® IC, supercritical CO₂/MeOH (+0.1% 7.0 M NH₃ in MeOH/MeOH) to give 2-((1R,2R)-2-aminocyclooctyl)-5-chloro-N-(thiophen-2-ylmethyl)thieno[3,2-b]pyridin-7-amine (Compound 103) 2.3 mg, 3% Yield), 2-((1S,2S)-2-aminocyclooctyl)-5-chloro-N-(thiophen-2-ylmethyl)thieno[3,2-b]pyridin-7-amine (Compound 104, 8.9 mg, 11% Yield), 2-((1R,2S)-2-aminocyclooctyl)-5-chloro-N-(thiophen-2-ylmethyl)thieno[3,2-b]pyridin-7-amine (Compound 105, 1.3 mg, 1.67% Yield) and 2-((1S,2R)-2-aminocyclooctyl)-5-chloro-N-(thiophen-2-ylmethyl)thieno[3,2-b]pyridin-7-amine (Compound 106, 18.1 mg, 23.21% Yield) as a white solid.

Compound 103: MS m/z 406.0 [M+H]⁺; ¹H NMR (METHANOL-d₄) δ ppm 7.29 (dd, J=5.1, 1.1 Hz, 1H), 7.14 (s, 1H), 7.07 (d, J=2.5 Hz, 1H), 6.97 (dd, J=5.0, 3.5 Hz, 1H), 6.51 (s, 1H), 4.75 (s, 2H), 3.41 (d, J=6.8 Hz, 1H), 3.21-3.08 (m, 1H), 2.14-1.84 (m, 6H), 1.77-1.58 (m, 6H) (3 exchangeable protons are not seen).

Compound 104: MS m/z 406.0 [M+H]⁺; ¹H NMR (METHANOL-d₄) δ ppm 7.29 (dd, J=5.1, 1.2 Hz, 1H), 7.12 (s, 1H), 7.07 (dd, J=3.5, 1.1 Hz, 1H), 6.97 (dd, J=5.1, 3.5 Hz, 1H), 6.49 (s, 1H), 4.74 (s, 2H), 3.50 (dt, J=10.7, 3.7 Hz, 1H), 3.42-3.34 (m, 1H), 2.21-2.09 (m, 1H), 1.99 (d, J=13.7 Hz, 1H), 1.91-1.66 (m, 10H) (3 exchangeable protons are not seen).

Compound 105: MS m/z 406.0 [M+H]⁺; ¹H NMR (METHANOL-d₄) δ ppm 7.29 (dd, J=5.1, 1.0 Hz, 1H), 7.18 (s, 1H), 7.08 (d, J=2.6 Hz, 1H), 6.97 (dd, J=5.0, 3.5 Hz, 1H), 6.53 (s, 1H), 4.75 (s, 2H), 3.63-3.55 (m, 1H), 3.26 (d, J=7.9 Hz, 1H), 2.15-1.93 (m, 6H), 1.70 (d, J=67.1 Hz, 6H) (3 exchangeable protons are not seen).

Compound 106: MS m/z 406.0 [M+H]; ¹H NMR (METHANOL-d₄) δ ppm 7.29 (d, J=4.6 Hz, 1H), 7.16 (s, 1H), 7.07 (s, 1H), 7.00-6.90 (m, 1H), 6.51 (s, 1H), 4.75 (s, 2H), 3.57 (s, 2H), 2.10 (s, 2H), 1.89 (s, 3H), 1.72 (d, J=38.6 Hz, 7H) (3 exchangeable protons are not seen).

The compounds below were prepared according to the procedure of Example 4 by substituting the appropriate starting materials, reagents, reaction conditions, and followed by chiral SFC resolution if needed.

| Compound | Spectral Data |
|---|---|
| 9 | MS m/z 363.9 [M + H]⁺; ¹H NMR (400 MHz, METHANOL-d₄) δ: ppm |
| | 7.29 (dd, J = 5.13, 1.13 Hz, 1 H) 7.11 (d, J = 1.38 Hz, 1 H) 7.05-7.08 (m, |
| | 1 H) 6.97 (dd, J = 5.00, 3.50 Hz, 1 H) 6.45-6.54 (m, 1 H) 4.74 (s, 2 H) |
| | 3.42-3.62 (m, 2 H) 2.14 (br s, 4 H) 1.61-1.87 (m, 2 H). (3 |
| | exchangeable protons are not seen) |
| 10 | MS m/z 364.0 [M + H]⁺; ¹H NMR (400 MHz, METHANOL-d₄) δ: ppm |
| | 7.29 (dd, J = 5.07, 1.19 Hz, 1 H) 7.11 (s, 1 H) 7.07 (dd, J = 3.44, 1.06 Hz, |
| | 1 H) 6.97 (dd, J = 5.13, 3.50 Hz, 1 H) 6.48 (s, 1 H) 4.74 (s, 2 H) 3.21- |
| | 3.27 (m, 1 H) 3.01-3.15 (m, 1 H) 2.10-2.38 (m, 2 H) 1.83-1.97 (m, 3 |
| | H) 1.51-1.69 (m, 1 H). (3 exchangeable protons are not seen) |
| 21 | MS m/z 378.1 [M + H]⁺; ¹HNMR(400 MHz, METHANOL-d₄) δ: 7.29 |
| | (dd, J = 5.1, 1.1 Hz, 1H), 7.13 (s, 1H), 7.07 (d, J = 2.5 Hz, 1H), 6.97 |
| | (dd, J = 5.1, 3.5 Hz, 1H), 6.50 (s, 1H), 4.75 (s, 2H), 2.95-2.87 (m, 1H), |
| | 2.80 (dd, J = 15.3, 6.9 Hz, 1H), 2.06 (d, J = 12.9 Hz, 2H), 1.85 (d, J = |
| | 12.2 Hz, 2H), 1.69-1.60 (m, 1H), 1.53-1.31 (m, 3H). (3 exchangeable |
| | protons are not seen) |
| 22 | MS m/z 378.1 [M + H]⁺; ¹HNMR(400 MHz, METHANOL-d₄) δ: 7.29 |
| | (dd, J = 5.1, 1.1 Hz, 1H), 7.11-7.03 (m, 2H), 6.96 (dt, J = 12.2, 6.1 Hz, |
| | 1H), 6.49 (s, 1H), 4.74 (s, 2H), 3.36 (t, J = 8.0 Hz, 1H), 3.29-3.22 (m, |
| | 1H), 1.93-1.82 (m, 3H), 1.71-1.46 (m, 3H), 1.38-1.25 (m, 2H). (3 |
| | exchangeable protons are not seen) |
| 23 | MS m/z 378.1 [M + H]⁺; ¹HNMR(400 MHz, METHANOL-d₄) δ: 7.29 |
| | (dd, J = 5.1, 1.2 Hz, 1H), 7.18-7.12 (m, 1H), 7.07 (dd, J = 3.5, 1.0 Hz, |
| | 1H), 6.97 (dd, J = 5.1, 3.5 Hz, 1H), 6.48 (d, J = 19.3 Hz, 1H), 4.74 (d, J = |
| | 6.9 Hz, 2H), 3.00 (td, J = 10.8, 3.8 Hz, 1H), 2.90-2.79 (m, 1H), 2.08 |
| | (d, J = 13.1 Hz, 2H), 1.88 (t, J = 12.6 Hz, 2H), 1.35-1.24 (m, 4H). (3 |
| | exchangeable protons are not seen) |
| 80 | MS m/z 392.2 [M + H]⁺; ¹HNMR(400 MHz, METHANOL-d₄) δ: 7.29 |
| | (dd, J = 5.1, 1.2 Hz, 1H), 7.12 (s, 1H), 7.07 (dd, J = 3.5, 1.0 Hz, 1H), |
| | 6.97 (dd, J = 5.1, 3.5 Hz, 1H), 6.50 (s, 1H), 4.74 (s, 2H), 3.16 (dd, J = |
| | 11.6, 6.5 Hz, 1H), 2.95 (td, J = 9.4, 3.3 Hz, 1H), 1.98-1.83 (m, 5H), |
| | 1.70 (d, J = 15.2 Hz, 5H). (3 exchangeable protons are not seen) |
| 88 | MS m/z 392.1 [M + H]⁺; ¹HNMR(400 MHz, METHANOL-d₄) δ: 7.29 |
| | (dd, J = 5.1, 1.2 Hz, 1H), 7.14 (s, 1H), 7.07 (dd, J = 3.4, 1.0 Hz, 1H), |
| | 6.97 (dd, J = 5.1, 3.5 Hz, 1H), 6.52 (s, 1H), 4.75 (s, 2H), 3.55 (d, J = 9.8 |
| | Hz, 2H), 2.14 (s, 1H), 2.04-1.84 (m, 6H), 1.59 (s, 3H). (3 |
| | exchangeable protons are not seen) |
| 89 | MS m/z 392.2 [M + H]⁺; ¹HNMR(400 MHz, METHANOL-d₄) δ: 7.29 |
| | (d, J = 4.9 Hz, 1H), 7.08 (d, J = 13.3 Hz, 2H), 7.00-6.95 (m, 1H), 6.49 |
| | (s, 1H), 4.74 (s, 2H), 3.41 (s, 2H), 1.91 (dd, J = 60.4, 36.1 Hz, 7H), 1.58 |
| | (s, 3H). (3 exchangeable protons are not seen) |
| 90 | MS m/z 392.1 [M + H]⁺; ¹HNMR(400 MHz, METHANOL-d₄) δ: 7.29 |
| | (dd, J = 5.1, 1.2 Hz, 1H), 7.17 (s, 1H), 7.07 (dd, J = 3.4, 1.0 Hz, 1H), |
| | 6.97 (dd, J = 5.1, 3.5 Hz, 1H), 6.53 (s, 1H), 4.75 (s, 2H), 3.46-3.36 (m, |
| | 1H), 3.13 (td, J = 9.7, 3.4 Hz, 1H), 2.09-1.84 (m, 6H), 1.71 (t, J = 8.2 |
| | Hz, 4H). (3 exchangeable protons are not seen) |
| 111 | MS m/z 364.1 [M + H]⁺; ¹H NMR(400 MHz, METHANOL-d₄) δ: 7.29 |
| | (dd, J = 5.1, 1.1 Hz, 1H), 7.11 (s, 1H), 7.07 (d, J = 2.5 Hz, 1H), 6.97 (dd, |
| | J = 5.1, 3.5 Hz, 1H), 6.49 (s, 1H), 4.74 (s, 2H), 3.55 (dd, J = 9.7, 5.8 Hz, |
| | 1H), 3.52-3.43 (m, 1H), 2.25-1.95 (m, 5H), 1.81 (dt, J = 13.9, 9.4 Hz, |
| | 1H), 1.68 (ddd, J = 12.8, 9.1, 4.7 Hz, 1H). (3 exchangeable protons are |
| | not seen) |
| 112 | MS m/z 364.1 [M + H]⁺; ¹H NMR (400 MHz, METHANOL-d₄) δ: 7.29 |
| | (dd, J = 5.1, 1.1 Hz, 1H), 7.11 (s, 1H), 7.07 (dd, J = 3.4, 1.0 Hz, 1H), |
| | 6.97 (dd, J = 5.1, 3.5 Hz, 1H), 6.48 (s, 1H), 4.74 (s, 2H), 3.55 (dd, J = |
| | 9.7, 5.8 Hz, 1H), 3.52-3.43 (m, 1H), 2.25-1.95 (m, 5H), 1.81 (dt, J = |
| | 13.9, 9.4 Hz, 1H), 1.68 (ddd, J = 12.8, 9.1, 4.7 Hz, 1H). (3 exchangeable |
| | protons are not seen) |

### Example 5

### Preparation of Compound 31

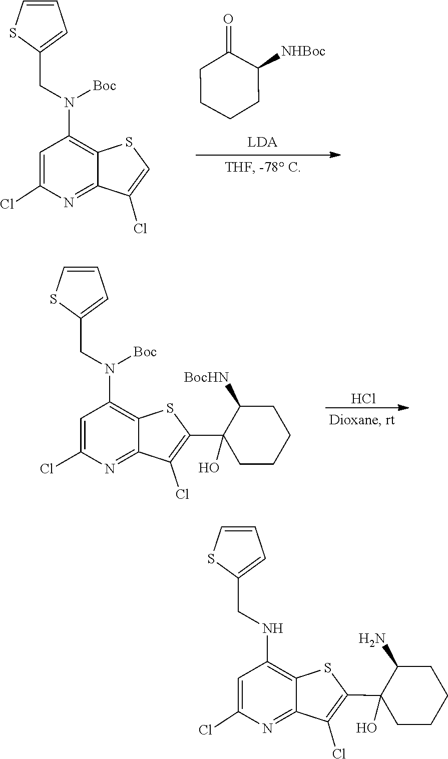 [SMILES: CC(C)(C)OC(=O)N(Cc1cccs1)c1cc(Cl)nc2c(Cl)csc12 ; CC(C)(C)OC(=O)N[C@H]1CCCCC1=O ; CC(C)(C)OC(=O)N[C@H]1CCCCC1(O)c1sc2c(N(Cc3cccs3)C(=O)OC(C)(C)C)cc(Cl)nc2c1Cl ; Cl ; N[C@H]1CCCCC1(O)c1sc2c(NCc3cccs3)cc(Cl)nc2c1Cl]

### Step 1: tert-Butyl (2-((2S)-2-((tert-butoxycarbonyl)amino)-1-hydroxycyclohexyl)-3,5-dichlorothieno[3,2-b]pyridin-7-yl)(thiophen-2-ylmethyl)carbamate

To a solution of tert-butyl N-(3,5-dichlorothieno[3,2-b]pyridin-7-yl)-N-(2-thienylmethyl)carbamate (200 mg, 0.4815 mmol) in THF (2 mL), cooled to −78° C., was added lithium diisopropylamide (2.0 M) in THF/heptane/ethylbenzene (0.31 mL, 0.62 mmol, 1.3 eq). After 15 min, a solution of (S)—N-Boc-2-aminocyclohexanone (140 mg, 0.630172 mmol, 1.3 eq) in THF (1 mL) was added to the reaction solution, and the mixture was stirred at room temperature for 30 min. The reaction was quenched with NH₄Cl (sat. aq.). The reaction mixture was diluted with EtOAc and washed with water and brine. The combined organic layers were dried over MgSO₄, filtered, and concentrated in vacuo. The crude residue was purified by flash column chromatography on silica gel eluting with ethyl acetate in hexanes (0-50% gradient) to afford tert-butyl N-[2-[(2S)-2-(tert-butoxycarbonylamino)-1-hydroxy-cyclohexyl]-3,5-dichloro-thieno[3,2-b]pyridin-7-yl]-N-(2-thienylmethyl)carbamate (75 mg, 25% Yield) as a clear oil. MS m/z 628.3, 630.1 [M+H]⁻, ¹H NMR (CHLOROFORM-d) δ: 7.29 (s, 1H), 7.24 (dd, J=5.1, 0.9 Hz, 1H), 7.03 (s, 1H), 6.90 (dd, J=5.0, 3.5 Hz, 1H), 6.80 (d, J=3.1 Hz, 1H), 5.05 (s, 2H), 4.21 (br s, 1H), 1.80-1.95 (m, 4H), 1.63-1.73 (m, 2H), 1.51-1.63 (m, 2H), 1.48 (s, 9H), 1.30 (s, 9H).

### Step 2: (2S)-2-Amino-1-(3,5-dichloro-7-((thiophen-2-ylmethyl)amino)thieno[3,2-b]pyridin-2-yl)cyclohexan-1-ol

General Boc-deprotection procedure: A solution of tert-butyl N-[2-[(2S)-2-(tert-butoxycarbonylamino)-1-hydroxy-cyclohexyl]-3,5-dichloro-thieno[3,2-b]pyridin-7-yl]-N-(2-thienylmethyl)carbamate (75 mg, 0.1193 mmol) and HCl (4 M) in dioxane (1 mL) was stirred at room temperature for 6 hr. Reaction mixture was diluted with diethyl ether and filtered. The filter cake was purified on preparative HPLC eluting with 10-100% ACN in water with 0.1% formic acid to afford (2S)-2-amino-1-[3,5-dichloro-7-(2-thienylmethylamino)thieno[3,2-b]pyridin-2-yl]cyclohexanol (28 mg, 50%). MS m/z 427.9, 429.8 [M+H]⁺, ¹H NMR (METHANOL-d₄) δ: 7.18-7.22 (m, 1H), 6.98 (d, J=2.6 Hz, 1H), 6.88 (dd, J=4.9, 3.7 Hz, 1H), 6.47 (s, 1H), 4.66 (s, 2H), 3.81 (dd, J=11.5, 4.3 Hz, 1H), 2.39-2.57 (m, 1H), 1.75-1.90 (m, 3H), 1.56-1.75 (m, 3H), 1.40-1.56 (m, 1H). HCl salt, (5 exchangeable protons are not seen).

### Example 6

### Preparation of Compound 32

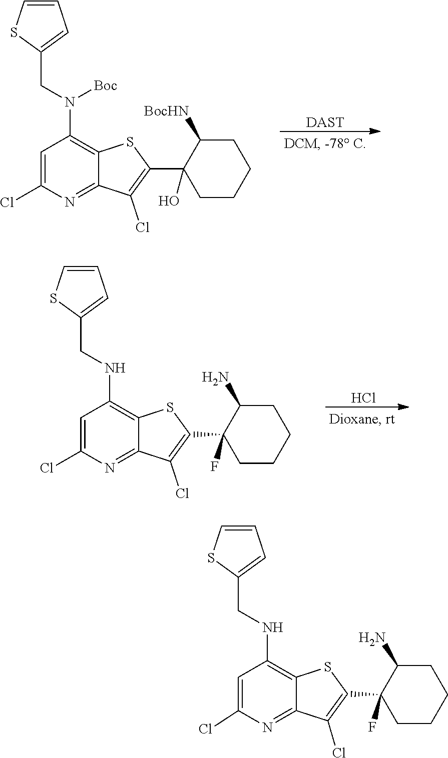

### Step 1: tert-Butyl (2-((1R,2S)-2-((tert-butoxycarbonyl)amino)-1-fluorocyclohexyl)-3,5-dichlorothieno[3,2-b]pyridin-7-yl)(thiophen-2-ylmethyl)carbamate

To a solution of tert-butyl N-[2-[(2S)-2-(tert-butoxycarbonylamino)-1-hydroxy-cyclohexyl]-3,5-dichloro-thieno[3,2-b]pyridin-7-yl]-N-(2-thienylmethyl)carbamate (120 mg, 0.2 mmol) in CH₂Cl₂ (2 mL), cooled to −78° C., was added diethylaminosulfur trifluoride (0.1 mL, 0.8 mmol, 3 eq). After stirring for 1 h at −78° C., the reaction was allowed to warm to rt for 30 min then quenched with NaHCO₃ (sat. aq.). The reaction mixture was diluted with DCM and the organic layer was separated, dried over Na₂SO₄, filtered, and concentrated in vacuo. The crude residue was purified by flash column chromatography on silica gel eluting with ethyl acetate in hexanes (0-40% gradient) to afford tert-butyl N-[2-[(2S)-2-(tert-butoxycarbonylamino)-1-fluoro-cyclohexyl]-3,5-dichloro-thieno[3,2-b]pyridin-7-yl]-N-(2-thienylmethyl)carbamate (58 mg, 48%) as a clear oil. MS m/z 630.1 632.2 [M+H]⁺, ¹H NMR (CHLOROFORM-d) δ: 7.17 (d, J=5.0 Hz, 1H), 6.99 (s, 1H), 6.79-6.84 (m, 1H), 6.73 (d, J=3.1 Hz, 1H), 5.41 (br s, 1H), 4.96 (s, 2H), 4.11-4.21 (m, 1H), 2.37-2.48 (m, 1H), 2.26-2.37 (m, 1H), 2.00-2.15 (m, 2H), 1.61-1.72 (m, 2H), 1.48-1.61 (m, 2H), 1.40 (s, 9H), 1.37 (s, 9H). Step 2: Deprotection of tert-butyl N-[2-[(2S)-2-(tert-butoxycarbonylamino)-1-fluoro-cyclohexyl]-3,5-dichloro-thieno[3,2-b]pyridin-7-yl]-N-(2-thienylmethyl)carbamate (58 mg, 0.1 mmol) using the general Boc deprotection procedure described in Step 2 of Example 5 gave 2-((1R,2S)-2-amino-1-fluorocyclohexyl)-3,5-dichloro-N-(thiophen-2-ylmethyl)thieno[3,2-b]pyridin-7-amine (34 mg, 85%). MS m/z 429.9, 431.8 [M+H]⁺, ¹H NMR (METHANOL-d₄) δ: 7.20 (d, J=5.1 Hz, 1H), 6.96-7.00 (m, 1H), 6.85-6.90 (m, 1H), 6.51 (s, 1H), 4.66 (s, 2H), 3.58-3.75 (m, 1H), 2.23-2.45 (m, 1H), 2.05 (br d, J=10.6 Hz, 1H), 1.87 (br d, J=11.4 Hz, 1H), 1.71-1.83 (m, 1H), 1.43-1.69 (m, 4H). HCl salt, (4 exchangeable protons are not seen).

The compounds below were prepared according to the procedure of Example 6 by substituting the appropriate starting materials, reagents, reaction conditions, and followed by chiral SFC resolution if needed.

| Compound | Spectral Data |
|---|---|
| 37 | MS m/z 467.9, 469.8, 471.9 [M + H]⁺; ¹H NMR (METHANOL-d4) δ: |
| | 7.24-7.43 (m, 5H), 6.54 (s, 1H), 4.37-4.54 (m, 1H), 2.52-2.77 (m, 1H), |
| | 2.15 (br d, J = 3.5 Hz, 2H), 1.64-2.07 (m, 5H). (4 exchangeable protons |
| | are not seen) |
| 38 | MS m/z 413.9, 415.8 [M + H]⁺; ¹H NMR (METHANOL-d4) δ: 7.47 (d, |
| | J = 1.0 Hz, 1H), 6.74 (s, 1H), 6.33-6.43 (m, 2H), 4.58 (s, 2H), 4.23-4.39 |
| | (m, 1H), 2.41-2.67 (m, 1H), 2.11-2.32 (m, 2H), 1.63-2.06 (m, 5H). (4 |
| | exchangeable protons are not seen) |
| 41 | MS m/z 394.0, 395.9 [M + H]⁺; ¹H NMR (400 MHz, METHANOL-d₄) δ: |
| | ppm 7.46 (d, J = 1.00 Hz, 1 H) 6.70 (s, 1 H) 6.28-6.43 (m, 2 H) 4.57 (s, |
| | 2 H) 3.83-4.04 (m, 1 H) 2.51 (s, 3 H) 2.07-2.37 (m, 3 H) 1.60-2.04 |
| | (m, 5 H). (4 exchangeable protons are not seen). |
| 121 | MS m/z 400.0 [M + H]⁺; ¹H NMR(400 MHz, METHANOL-d₄) δ: 7.45 |
| | (d, J = 1.0 Hz, 1H), 6.67 (d, J = 10.3 Hz, 1H), 6.36 (dd, J = 3.2, 1.9 Hz, |
| | 1H), 6.33 (d, J = 3.1 Hz, 1H), 4.54 (s, 2H), 3.91 (ddd, J = 25.3, 11.3, 7.7 |
| | Hz, 1H), 2.77 (d, J = 38.9 Hz, 1H), 2.42-2.12 (m, 2H), 2.04-1.84 (m, |
| | 2H), 1.83-1.66 (m, 1H). (3 exchangeable protons are not seen). |
| 128 | MS m/z 381.1 [M + H]⁺; ¹H NMR(400 MHz, METHANOL-d₄) δ: 7.42 |
| | (dd, J = 1.8, 0.8 Hz, 1H), 6.38-6.29 (m, 2H), 4.74 (s, 2H), 3.77-3.60 |
| | (m, 1H), 2.48 (dd, J = 12.0, 6.0 Hz, 1H), 2.39 (dd, J = 14.5, 3.6 Hz, 4H), |
| | 2.23 (ddd, J = 11.3, 7.9, 3.3 Hz, 1H), 2.06-1.86 (m, 2H), 1.86-1.73 |
| | (m, 1H). (3 exchangeable protons are not seen). |

### Example 7

### Preparation of Compounds 150 and 151

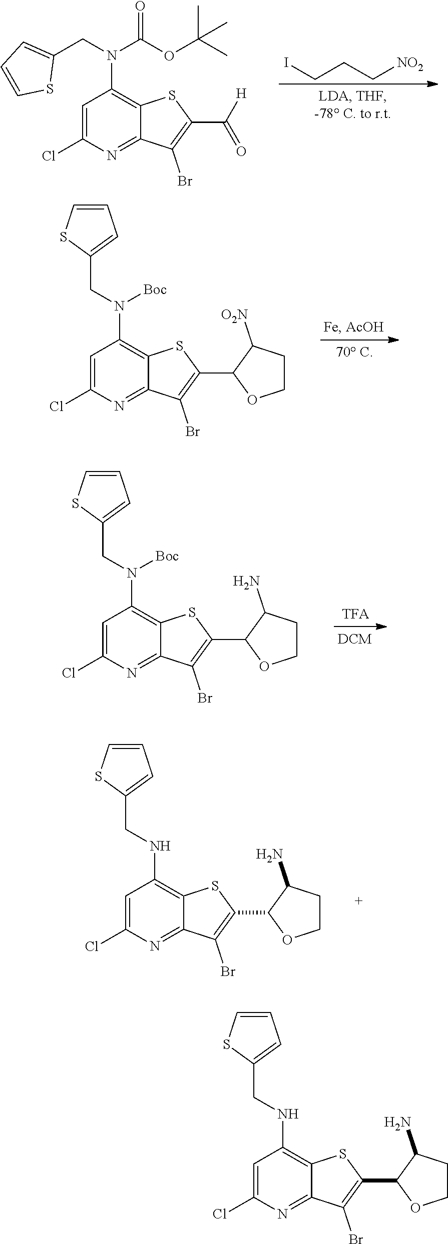

### Step 1: tert-butyl (3-bromo-5-chloro-2-(3-nitrotetrahydrofuran-2-yl)thieno[3,2-b]pyridin-7-yl)(thiophen-2-ylmethyl)carbamate

To a THF (10 mL) solution of tert-butyl (3-bromo-5-chloro-2-formylthieno[3,2-b]pyridin-7-yl)(thiophen-2-ylmethyl)carbamate (488 mg, 1.0 mmol, 1.0 eq.), prepared according to a previous procedure, was added 1-iodo-3-nitropropane (323 mg, 1.5 mmol, 1.5 eq.). The mixture was cooled to −78° C., at which point lithium diisopropylamide (1 M in hexanes, 1.3 mL, 1.3 mmol, 1.3 eq.) was added dropwise. The mixture was warmed to room temperature overnight. The mixture was then diluted with ethyl acetate and washed with water and brine. The organic layer was dried over Na₂SO₄, filtered and concentrated in vacuo. The residue was purified by silica gel column chromatography with ethyl acetate in hexanes (5 to 35% gradient) to give tert-butyl (3-bromo-5-chloro-2-(3-nitrotetrahydrofuran-2-yl)thieno[3,2-b]pyridin-7-yl)(thiophen-2-ylmethyl)carbamate as a mixture of diastereomers (368 mg, 64% yield). MS m/z 576.0 [M+H]⁺.

### Step 2: tert-Butyl (2-(3-aminotetrahydrofuran-2-yl)-3-bromo-5-chlorothieno[3,2-b]pyridin-7-yl)(thiophen-2-ylmethyl)carbamate

tert-Butyl (3-bromo-5-chloro-2-(3-nitrotetrahydrofuran-2-yl)thieno[3,2-b]pyridin-7-yl)(thiophen-2-ylmethyl)carbamate as a mixture of diastereomers (273 mg, 0.5 mmol, 1.0 eq.) was mixed with iron powder (280 mg, 5 mmol, 10.0 eq.) and acetic acid (5 mL). The mixture was heated at 70° C. for 2 h. After the reaction cooled down, the mixture was filtered and concentrated in vacuo. The residue was purified by silica gel column chromatography with methanol (0.1% NH₃) in DCM (0 to 20% gradient) to give tert-butyl (2-(3-aminotetrahydrofuran-2-yl)-3-bromo-5-chlorothieno[3,2-b]pyridin-7-yl)(thiophen-2-ylmethyl)carbamate as a diastereomeric mixture (218 mg, 80% yield). MS m/z 546.0 [M+H]⁺.

### Step 3: 2-(trans-3-aminotetrahydrofuran-2-yl)-3-bromo-5-chloro-N-(thiophen-2-ylmethyl)thieno[3,2-b]pyridin-7-amine and 2-(cis-3-aminotetrahydrofuran-2-yl)-3-bromo-5-chloro-N-(thiophen-2-ylmethyl)thieno[3,2-b]pyridin-7-amine

To a DCM (2 mL) solution of tert-butyl (2-(3-aminotetrahydrofuran-2-yl)-3-bromo-5-chlorothieno[3,2-b]pyridin-7-yl)(thiophen-2-ylmethyl)carbamate (55 mg, 0.1 mmol, 1.0 eq.) was added trifluoroacetic acid (2 mL) at room temperature. After stirring for 1 h, the mixture was concentrated in vacuo. The residue was purified by reverse phase column chromatography with acetonitrile (0.1% formic acid) in water (0.1% formic acid) (0 to 40% gradient) to give 2-(trans-3-aminotetrahydrofuran-2-yl)-3-bromo-5-chloro-N-(thiophen-2-ylmethyl)thieno[3,2-b]pyridin-7-amine and 2-(cis-3-aminotetrahydrofuran-2-yl)-3-bromo-5-chloro-N-(thiophen-2-ylmethyl)thieno[3,2-b]pyridin-7-amine as two separate diastereomers.

Compound 150: MS m/z 446.0 [M+H]⁺; ¹H NMR (METHANOL-d₄) δ: 8.36 (s, 1H), 7.32 (d, J=5.1 Hz, 1H), 7.12-7.08 (m, 1H), 7.03-6.97 (m, 1H), 6.63 (s, 1H), 5.41 (s, 1H), 4.78 (s, 2H), 4.38 (td, J=8.8, 4.2 Hz, 1H), 4.23 (tt, J=8.3 Hz, 1H), 4.05-3.98 (m, 1H), 2.55-2.42 (m, 1H), 2.20-2.10 (m, 1H). formic acid salt, (4 exchangeable protons are not seen).

Compound 151: MS m/z 446.0 [M+H]⁺; ¹H NMR (METHANOL-d₄) δ: 8.35 (s, 1H), 7.30 (dd, J=5.1, 1.2 Hz, 1H), 7.12-7.06 (m, 1H), 6.98 (dd, J=5.1, 3.5 Hz, 1H), 6.63 (s, 1H), 5.36 (d, J=4.1 Hz, 1H), 4.77 (s, 2H), 4.41-4.26 (m, 2H), 4.04 (td, J=9.4, 5.3 Hz, 1H), 2.67 (dt, J=9.4, 7.0 Hz, 1H), 2.22-2.12 (m, 1H). formic acid salt, (4 exchangeable protons are not seen).

### Example 8

### Preparation of Compounds 46 and 47

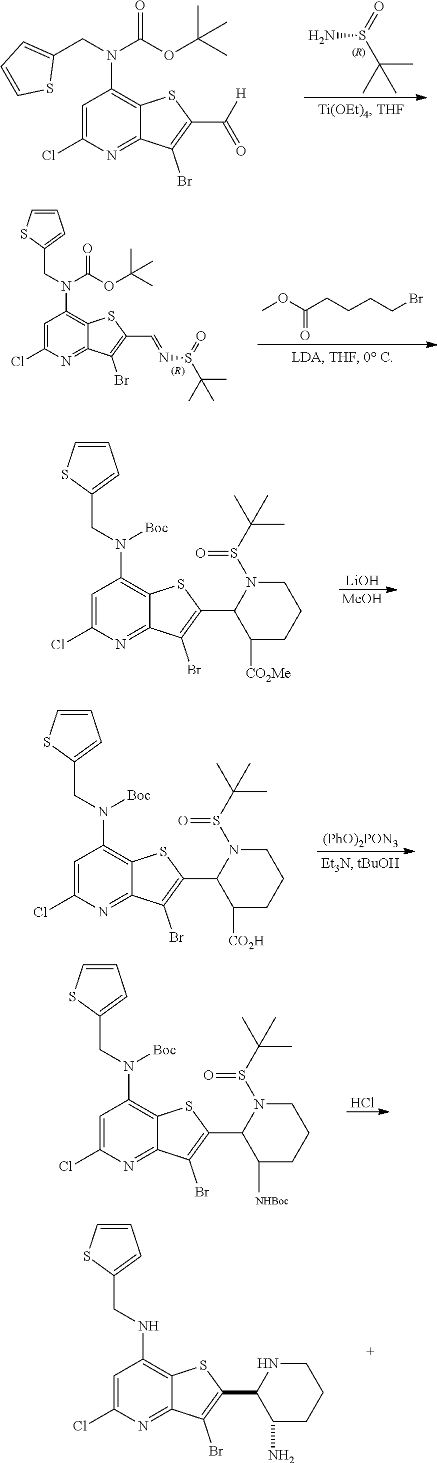

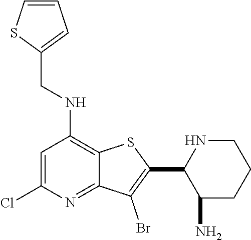

### Step 1: tert-Butyl (R,E)-(3-bromo-2-(((tert-butylsulfinyl)imino)methyl)-5-chlorothieno[3,2-b]pyridin-7-yl)(thiophen-2-ylmethyl)carbamate

tert-Butyl (3-bromo-5-chloro-2-formylthieno[3,2-b]pyridin-7-yl)(thiophen-2-ylmethyl)carbamate (488 mg, 1.0 mmol, 1.0 eq.), prepared according to a previous procedure, was dissolved in THF (5 mL). Ti(OEt)₄ (456 mg, 2.0 mmol, 2.0 eq.) and (R)-2-methylpropane-2-sulfinamide (182 mg, 1.5 mmol, 1.5 eq.) were added and the vial was heated to 50° C. Conversion was monitored by LCMS, and the mixture was cooled to room temperature after completion. Once at room temperature, the mixture was poured into an equal volume of brine while rapidly stirring. The resulting suspension was filtered through a plug of Celite, and the filter cake was washed with EtOAc. The filtrate was transferred to a separatory funnel where the organic layer was washed with brine. The brine layer was extracted once with a small volume of EtOAc, and the combined organic portions were dried over Na₂SO₄, filtered and concentrated in vacuo. The residue was purified by silica gel column chromatography with ethyl acetate in hexanes (5 to 100% gradient) to give tert-butyl (R,E)-(3-bromo-2-(((tert-butylsulfinyl)imino)methyl)-5-chlorothieno[3,2-b]pyridin-7-yl)(thiophen-2-ylmethyl)carbamate (561 mg, 95% yield). MS m/z 592.0 [M+H]⁺.

### Step 2: Methyl 2-(3-bromo-7-((tert-butoxycarbonyl)(thiophen-2-ylmethyl)amino)-5-chlorothieno[3,2-b]pyridin-2-yl)-1-(tert-butylsulfinyl)piperidine-3-carboxylate

To a mixture of tert-butyl (R,E)-(3-bromo-2-(((tert-butylsulfinyl)imino)methyl)-5-chlorothieno[3,2-b]pyridin-7-yl)(thiophen-2-ylmethyl)carbamate (473 mg, 0.8 mmol, 1.0 eq.) in THF (8 mL) was added methyl 5-bromovalerate (234 mg, 1.2 mmol, 1.5 eq.). The reaction mixture was cooled to 0° C. Lithium diisopropylamide (1M in hexanes, 1.0 mL, 1.3 eq.) was added dropwise to the reaction. LCMS showed the reaction completed after 1 h. The reaction was quenched with saturated NH₄Cl then diluted with ethyl acetate and washed with brine. The organic layer was dried over Na₂SO₄, filtered and concentrated in vacuo. The residue was purified by silica gel column chromatography with ethyl acetate in hexanes (5 to 50% gradient) to give methyl 2-(3-bromo-7-((tert-butoxycarbonyl)(thiophen-2-ylmethyl)amino)-5-chlorothieno[3,2-b]pyridin-2-yl)-1-(tert-butylsulfinyl)piperidine-3-carboxylate (197 mg, 35% yield) as a diastereomeric mixture. MS m/z 706.1 [M+H]⁺.

### Step 3: 2-(3-Bromo-7-((tert-butoxycarbonyl)(thiophen-2-ylmethyl)amino)-5-chlorothieno[3,2-b]pyridin-2-yl)-1-(tert-butylsulfinyl)piperidine-3-carboxylic acid

To a solution of methyl 2-(3-bromo-7-((tert-butoxycarbonyl)(thiophen-2-ylmethyl)amino)-5-chlorothieno[3,2-b]pyridin-2-yl)-1-(tert-butylsulfinyl)piperidine-3-carboxylate (353 mg, 0.5 mmol. 1.0 eq.) in MeOH (5 mL) was added lithium hydroxide (24 mg, 1.0 mmol, 2.0 eq.). The reaction mixture was allowed to stir at 50° C. for 4 h. The reaction was quenched with saturated NH₄Cl then diluted with ethyl acetate and washed with brine. The organic layer was dried over Na₂SO₄, filtered, and concentrated in vacuo. The residue was purified by silica gel column chromatography with ethyl acetate in hexanes (5 to 100% gradient) to give 2-(3-bromo-7-((tert-butoxycarbonyl)(thiophen-2-ylmethyl)amino)-5-chlorothieno[3,2-b]pyridin-2-yl)-1-(tert-butylsulfinyl)piperidine-3-carboxylic acid (296 mg, 84% yield) as a mixture of diastereomers. MS m/z 706.1 [M+H]⁺.

### Step 4: tert-Butyl (3-bromo-2-(3-((tert-butoxycarbonyl)amino)-1-(tert-butylsulfinyl)piperidin-2-yl)-5-chlorothieno[3,2-b]pyridin-7-yl)(thiophen-2-ylmethyl)carbamate

To a solution of 2-(3-bromo-7-((tert-butoxycarbonyl)(thiophen-2-ylmethyl)amino)-5-chlorothieno[3,2-b]pyridin-2-yl)-1-(tert-butylsulfinyl)piperidine-3-carboxylic acid (141 mg, 0.2 mmol, 1.0 eq.) in tert-butanol (2 mL) was added triethylamine (40 mg, 0.4 mmol, 2.0 eq.) and diphenylphosphoryl azide (83 mg, 0.3 mmol, 1.5 eq.). The reaction was allowed to stir at 90° C. for 3 h. The reaction was quenched with saturated NH₄Cl then diluted with ethyl acetate and washed with brine. The organic layer was dried over Na₂SO₄, filtered and concentrated in vacuo. The residue was purified by silica gel column chromatography with ethyl acetate in hexanes (5 to 50% gradient) to give tert-butyl (3-bromo-2-(3-((tert-butoxycarbonyl)amino)-1-(tert-butylsulfinyl)piperidin-2-yl)-5-chlorothieno[3,2-b]pyridin-7-yl)(thiophen-2-ylmethyl)carbamate (70 mg, 46% yield) as a mixture of diastereomers. MS m/z 763.1 [M+H]⁺.

### Step 5: 2-(trans-3-Aminopiperidin-2-yl)-3-bromo-5-chloro-N-(thiophen-2-ylmethyl)thieno[3,2-b]pyridin-7-amine and 2-(cis-3-aminopiperidin-2-yl)-3-bromo-5-chloro-N-(thiophen-2-ylmethyl)thieno[3,2-b]pyridin-7-amine

To a DCM (2 mL) solution of tert-butyl (3-bromo-2-(3-((tert-butoxycarbonyl)amino)-1-(tert-butylsulfinyl)piperidin-2-yl)-5-chlorothieno[3,2-b]pyridin-7-yl)(thiophen-2-ylmethyl)carbamate (152 mg, 0.2 mmol, 1.0 eq.) from Step 4 was added 2M HCl in dioxane (1 mL, 2 mmol, 10 eq.) at room temperature. After stirring for 1 h, the mixture was concentrated in vacuo. The residue was purified by reverse phase column chromatography with acetonitrile (0.10% formic acid) in water (0.1% formic acid) (0 to 40% gradient) to give 2-(trans-3-aminopiperidin-2-yl)-3-bromo-5-chloro-N-(thiophen-2-ylmethyl)thieno[3,2-b]pyridin-7-amine and 2-(cis-3-aminopiperidin-2-yl)-3-bromo-5-chloro-N-(thiophen-2-ylmethyl)thieno[3,2-b]pyridin-7-amine as two separate diastereomers.

Compound 46: MS m/z 459.0 [M+H]⁺; ¹H NMR (400 MHz, METHANOL-d₄) δ 8.55 (s, 1H), 7.30 (d, J=5.2 Hz, 1H), 7.08 (d, J=3.5 Hz, 1H), 6.98 (dd, J=5.2, 3.5 Hz, 1H), 6.57 (s, 1H), 4.75 (s, 2H), 4.50 (d, J=2.3 Hz, 1H), 3.53-3.47 (m, 1H), 3.17 (d, J=12.1 Hz, 1H), 2.86 (td, J=11.9, 3.0 Hz, 1H), 2.05-1.75 (m, 3H), 1.62-1.52 (m, 1H). formic acid salt, (5 exchangeable protons not seen).

Compound 47: MS m/z 459.0 [M+H]⁺; ¹H NMR (400 MHz, METHANOL-d₄) δ 8.55 (s, 1H), 7.30 (d, J=5.2 Hz, 1H), 7.09 (d, J=3.5 Hz, 1H), 6.98 (dd, J=5.2, 3.5 Hz, 1H), 6.60 (s, 1H), 4.76 (s, 2H), 4.53 (d, J=2.3 Hz, 1H), 3.63-3.55 (m, 1H), 3.18 (d, J=11.8 Hz, 1H), 2.88 (dd, J=13.2, 10.2 Hz, 1H), 2.07-1.90 (m, 2H), 1.89-1.75 (m, 1H), 1.65-1.55 (m, 1H). formic acid salt, (5 exchangeable protons not seen)

The compounds below were prepared according to the procedure of Example 8 by substituting the appropriate starting materials, reagents, reaction conditions, and followed by chiral SFC resolution if needed.

| Compound | Spectral Data |
|---|---|
| 124 | MS m/z 535.0 [M + H]⁺; ¹H NMR (METHANOL-d₄) δ: 8.53 (s, 0.6H, |
| | formic acid), 7.27 (dd, J = 5.1, 1.2 Hz, 1H), 7.20-7.08 (m, 4H), 7.05- |
| | 6.99 (m, 1H), 6.97-6.87 (m, 2H), 6.49 (s, 1H), 4.81 (d, J = 7.9 Hz, |
| | 1H), 4.67 (s, 2H), 3.50-3.34 (m, 2H), 3.01 (ddd, J = 13.1, 9.0, 4.3 Hz, |
| | 1H), 2.23 (ddd, J = 13.1, 4.3, 4.3 Hz, 1H), 2.08-1.90 (m, 2H), 1.79- |
| | 1.65 (m, 1H). (3 Exchangeable protons not observed). |
| 125 | MS m/z 535.0 [M + H]⁺; ¹H NMR (METHANOL-d₄) δ: 7.28 (d, J = 5.0 |
| | Hz, 1H), 7.20 (t, J = 7.9 Hz, 2H), 7.09 (d, J = 7.9 Hz, 2H), 7.05-7.00 |
| | (m, 1H), 6.98-6.91 (m, 2H), 6.55 (s, 1H), 5.51 (d, J = 3.4 Hz, 1H), |
| | 4.75-4.60 (m, 2H), 3.95-3.88 (m, 1H), 3.62-3.52 (m, 1H), 3.18- |
| | 3.05 (m, 1H), 2.22-2.06 (m, 3H), 1.92-1.80 (m, 1H). (3 |
| | Exchangeable protons not observed). |

### Example 9

### Preparation of Compounds 147 and 148

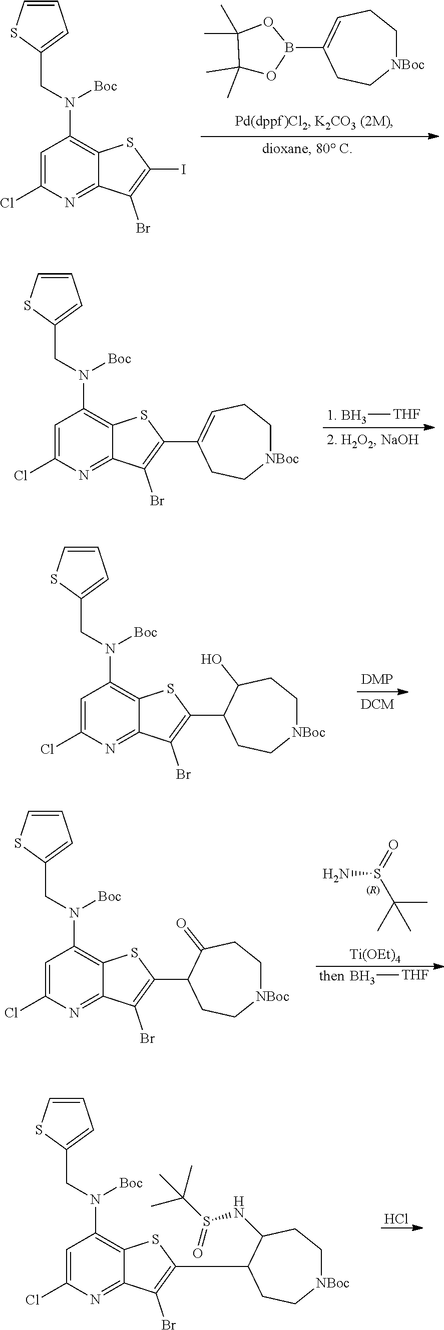

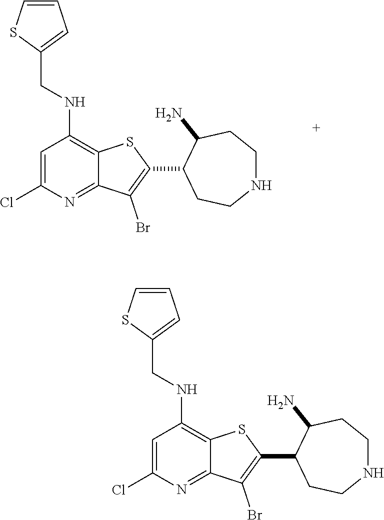

### Step 1: tert-Butyl 4-(3-bromo-7-((tert-butoxycarbonyl)(thiophen-2-ylmethyl)amino)-5-chlorothieno[3,2-b]pyridin-2-yl)-2,3,6,7-tetrahydro-1H-azepine-1-carboxylate

tert-Butyl (3-bromo-5-chloro-2-iodothieno[3,2-b]pyridin-7-yl)(thiophen-2-ylmethyl)carbamate (584 mg, 1 mmol, 1.0 eq.) (prepared following a previously described example) and tert-butyl 4-(4,4,5,5-tetramethyl-1,3,2-dioxaborolan-2-yl)-2,3,6,7-tetrahydro-1H-azepine-1-carboxylate (420 mg, 1.3 mmol, 1.3 eq.) were mixed in 10 mL dioxane. Catalyst Pd(dppf)Cl₂ (73 mg, 0.1 mmol, 0.1 eq.) and 2 M aqueous K₂CO₃ solution (1.25 mL, 2.5 eq.) were added to the reaction. The reaction mixture was purged by argon vigorously for 5 min. The reaction was allowed to stir at 80° C. for 6 h. The mixture was cooled to room temperature and diluted with ethyl acetate and washed with water and brine. The organic layer was dried over Na₂SO₄, filtered and concentrated in vacuo. The residue was purified by silica gel column chromatography with ethyl acetate in hexanes (5 to 60% gradient) to give tert-butyl 4-(3-bromo-7-((tert-butoxycarbonyl)(thiophen-2-ylmethyl)amino)-5-chlorothieno[3,2-b]pyridin-2-yl)-2,3,6,7-tetrahydro-1H-azepine-1-carboxylate (537 mg, 82% yield). MS m/z 656.1 [M+H]⁺.

### Step 2: tert-Butyl 4-(3-bromo-7-((tert-butoxycarbonyl)(thiophen-2-ylmethyl)amino)-5-chlorothieno[3,2-b]pyridin-2-yl)-5-hydroxyazepane-1-carboxylate

A THF solution (6 mL) of tert-butyl 4-(3-bromo-7-((tert-butoxycarbonyl)(thiophen-2-ylmethyl)amino)-5-chlorothieno[3,2-b]pyridin-2-yl)-2,3,6,7-tetrahydro-1H-azepine-1-carboxylate (524 mg, 0.8 mmol, 1.0 eq.) was cooled to 0° C. 1 M of borane THF complex solution (2.4 mL, 2.4 mmol, 3.0 eq.) was added to the reaction dropwise. The reaction was allowed to warm to room temperature overnight. LCMS showed complete consumption of starting material. The reaction was cooled to 0° C., to which was added 2 mL ethanol, 2 M aqueous sodium hydroxide (1 mL) and 30% aq H₂O₂ (1 mL) sequentially. The reaction was allowed to warm up to room temperature and stir for an additional 2 h. The reaction mixture was then poured into a separatory funnel and diluted with CH₂Cl₂. The water layer was washed with CH₂Cl₂ and the organic layers were combined and dried with Na₂SO₄. After filtration and removal of the solvent in vacuo, the crude mixture was purified by silica gel chromatography to give tert-butyl 4-(3-bromo-7-((tert-butoxycarbonyl)(thiophen-2-ylmethyl)amino)-5-chlorothieno[3,2-b]pyridin-2-yl)-5-hydroxyazepane-1-carboxylate (188 mg, 35% yield). MS m/z 674.1 [M+H]⁺.

### Step 3: tert-Butyl 4-(3-bromo-7-((tert-butoxycarbonyl)(thiophen-2-ylmethyl)amino)-5-chlorothieno[3,2-b]pyridin-2-yl)-5-oxoazepane-1-carboxylate

To a DCM (5 mL) solution of tert-butyl 4-(3-bromo-7-((tert-butoxycarbonyl)(thiophen-2-ylmethyl)amino)-5-chlorothieno[3,2-b]pyridin-2-yl)-5-hydroxyazepane-1-carboxylate (337 mg, 0.5 mmol, 1.0 eq.) was added Dess-Martin periodinane (318 mg, 0.75 mmol, 1.5 eq.). The reaction was allowed to stir at room temperature for 1 h, and quenched with saturated NaHCO₃ and saturated Na₂S₂O₃. The reaction mixture was then poured into a separatory funnel and diluted with CH₂Cl₂. The water layer was washed with CH₂Cl₂ and the organic layers were combined and dried with Na₂SO₄. After filtration and removal of the solvent in vacuo, the crude mixture was purified by silica gel chromatography to give tert-butyl 4-(3-bromo-7-((tert-butoxycarbonyl)(thiophen-2-ylmethyl)amino)-5-chlorothieno[3,2-b]pyridin-2-yl)-5-oxoazepane-1-carboxylate (285 mg, 85% yield). MS m/z 672.1 [M+H]⁺.

### Step 4: tert-Butyl 4-(3-bromo-7-((tert-butoxycarbonyl)(thiophen-2-ylmethyl)amino)-5-chlorothieno[3,2-b]pyridin-2-yl)-5-((tert-butylsulfinyl)amino)azepane-1-carboxylate

tert-Butyl 4-(3-bromo-7-((tert-butoxycarbonyl)(thiophen-2-ylmethyl)amino)-5-chlorothieno[3,2-b]pyridin-2-yl)-5-oxoazepane-1-carboxylate (168 mg, 0.25 mmol, 1.0 eq.) and (R)-2-methylpropane-2-sulfinamide (61 mg, 0.5 mmol, 2.0 eq.) were mixed in Ti(OEt)₄ (3 mL) and heated to 50° C. overnight. After the reaction mixture was cooled to room temperature, THF was added followed by 1 M of borane THF complex solution (0.4 mL, 0.4 mmol, 1.6 eq.). The reaction was complete in 10 minutes. The mixture was added to a stirring solution of aqueous K₂CO₃. After filtration and removal of the solvent, the crude mixture was purified by silica gel chromatography to give tert-butyl 4-(3-bromo-7-((tert-butoxycarbonyl)(thiophen-2-ylmethyl)amino)-5-chlorothieno[3,2-b]pyridin-2-yl)-5-((tert-butylsulfinyl)amino)azepane-1-carboxylate (146 mg, 75% yield) as a mixture of diastereomers. MS m/z 777.1 [M+H]⁺.

### Step 5: 2-(trans-5-Aminoazepan-4-yl)-3-bromo-5-chloro-N-(thiophen-2-ylmethyl)thieno[3,2-b]pyridin-7-amine and 2-(cis-5-aminoazepan-4-yl)-3-bromo-5-chloro-N-(thiophen-2-ylmethyl)thieno[3,2-b]pyridin-7-amine

To a DCM (2 mL) solution of tert-butyl 4-(3-bromo-7-((tert-butoxycarbonyl)(thiophen-2-ylmethyl)amino)-5-chlorothieno[3,2-b]pyridin-2-yl)-5-((tert-butylsulfinyl)amino)azepane-1-carboxylate (78 mg, 0.1 mmol, 1.0 eq.) was added 2M HCl in dioxane (1 mL, 2 mmol, 20 eq.) at room temperature. After stirring for 1 h, the mixture was concentrated in vacuo. The residue was purified by reverse phase column chromatography with acetonitrile (0.1% formic acid) in water (0.1% formic acid) (0 to 40% gradient) to 2-(trans-5-aminoazepan-4-yl)-3-bromo-5-chloro-N-(thiophen-2-ylmethyl)thieno[3,2-b]pyridin-7-amine and 2-(cis-5-aminoazepan-4-yl)-3-bromo-5-chloro-N-(thiophen-2-ylmethyl)thieno[3,2-b]pyridin-7-amine as two separate diastereomers. MS m/z 473.0 [M+H]⁺.

Compound 147: MS m/z 473.0 [M+H]⁺; ¹H NMR (DMSO-d₆) δ: 9.25 (brs, 1H), 8.10 (t, J=6.0 Hz, 2H), 7.41 (d, J=5.1 Hz, 1H), 7.11 (d, J=3.5 Hz, 1H), 6.99 (dd, J=5.1, 3.5 Hz, 1H), 6.69 (s, 1H), 4.83-4.67 (m, 2H), 4.20-4.05 (m, 1H), 4.05-3.96 (m, 1H), 3.80-3.65 (m, 2H), 3.30-3.18 (m, 1H), 3.07 (t, J=12.0 Hz, 1H), 2.37-2.27 (m, 2H), 2.24-2.12 (m, 1H), 2.10-2.00 (m, 1H), 1.98-1.85 (m, 1H).

Compound 148: MS m/z 473.0 [M+H]⁺; ¹H NMR (DMSO-d₆) δ: 9.43 (brs, 1H, TFA peak), 9.21 (brs, 1H), 8.21 (brs, 2H), 8.07 (t, J=6.0 Hz, 1H), 7.41 (d, J=5.0 Hz, 1H), 7.11 (d, J=3.5 Hz, 1H), 7.00 (dd, J=5.0, 3.5 Hz, 1H), 6.70 (s, 1H), 4.85-4.69 (m, 2H), 3.95-3.85 (m, 1H), 3.73-3.63 (m, 2H), 3.35-3.25 (m, 2H), 3.19 (t, J=11.0 Hz, 1H), 2.10-2.02 (m, 2H), 2.02-1.88 (m, 2H).

### Example 10

### Preparation of Compound 149

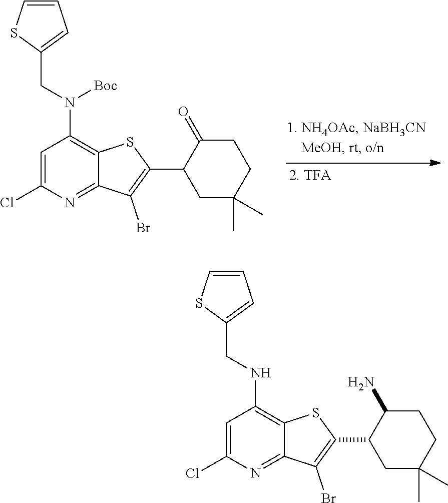

### Step 1: 2-(trans-2-Amino-5,5-dimethylcyclohexyl)-3-bromo-5-chloro-N-(thiophen-2-ylmethyl)thieno[3,2-b]pyridin-7-amine

tert-Butyl (3-bromo-5-chloro-2-(5,5-dimethyl-2-oxocyclohexyl)thieno[3,2-b]pyridin-7-yl)(thiophen-2-ylmethyl)carbamate (117 mg, 0.2 mmol) was prepared according to the procedure of Example 9 by substituting the appropriate starting materials. The reductive amination/deprotection procedure followed Example 11, which afforded 2-(trans-2-amino-5,5-dimethylcyclohexyl)-3-bromo-5-chloro-N-(thiophen-2-ylmethyl)thieno[3,2-b]pyridin-7-amine (15 mg, 15% yield). MS m/z 486.0 [M+H]⁺. ¹H NMR (METHANOL-d₄) δ: 8.54 (s, 1H, formic acid), 7.30 (d, J=5.1 Hz, 1H), 7.09 (d, J=3.5 Hz, 1H), 6.98 (dd, J=5.1, 3.5 Hz, 1H), 6.60 (s, 1H), 4.76 (s, 2H), 3.60 (td, J=11.7, 3.8 Hz, 1H), 3.18 (d, J=11.7 Hz, 1H), 1.99 (dt, J=13.2, 3.4 Hz, 1H), 1.82-1.69 (m, 2H), 1.66-1.46 (m, 3H), 1.14 (s, 3H), 1.03 (s, 3H). formic acid salt, (4 exchangeable protons not observed).

The compounds below were prepared according to Example 10 by substituting the appropriate starting materials, reagents, reaction conditions, and followed by chiral SFC resolution if needed.

| Compound | Spectral Data |
|---|---|
| 30 | MS m/z 491.9, 493.8, 495.8 [M + H]⁺; ¹H NMR (METHANOL-d₄) δ: |
| | 7.36-7.32 (m, 1H), 7.13 (d, J = 2.63 Hz, 1H), 7.01 (dd, J = 5.0, 3.6 Hz, |
| | 1H), 6.80 (s, 1H), 4.86-4.82 (m, 2H), 4.25 (br s, 1H), 4.02 (dt, J = 12.1, |
| | 3.9 Hz, 1H), 2.75-2.17 (m, 6H). (4 exchangeable protons not observed). |
| 152 | MS m/z 486.0 [M + H]⁺; ¹H NMR (METHANOL-d₄) δ: 8.54 (s, 1H, |
| | formic acid), 7.30 (dd, J = 5.1, 1.4 Hz, 1H), 7.09 (dd, J = 3.6, 1.4 Hz, |
| | 1H), 6.98 (dd, J = 5.1, 3.6 Hz, 1H), 6.60 (s, 1H), 4.76 (s, 2H), 4.10 (d, |
| | J = 4.8 Hz, 1H), 3.61 (dt, J = 10.4, 4.8 Hz, 1H), 2.19-2.06 (m, 1H), 1.96- |
| | 1.90 (m, 1H), 1.90-1.76 (m, 2H), 1.68-1.54 (m, 1H), 1.50-1.35 |
| | (m, 1H), 1.15 (s, 3H), 1.09 (s, 3H). Formic acid salt, (4 exchangeable |
| | protons not observed). |
| 204 | MS m/z 458.0, 460.0, 462.0 [M + H]⁺; ¹H NMR (METHANOL-d₄) δ: |
| | 7.33 (t, J = 5.3 Hz, 1H), 7.11 (br s, 1H), 6.93-7.04 (m, 1H), 6.66 (d, |
| | J = 15.6 Hz, 1H), 4.79 (d, J = 4.6 Hz, 2H), 4.08-4.28 (m, 2H), 3.88-4.01 |
| | (m, 1H), 3.61-3.82 (m, 3H), 2.13-2.28 (m, 1H), 1.85-1.96 (m, 1H). (4 |
| | exchangeable protons not observed). |
| 333 | MS m/z 428.1 [M + H]⁺; ¹H NMR (METHANOL-d₄) δ: 7.30-7.29 (m, |
| | 1H), 7.07 (s, 1H), 6.99-6.97 (m, 1H). 6.53 (s, 1H), 4.75 (s, 2H), 3.24 |
| | (br s, 1H), 3.17 (br s, 1H), 2.38 (s, 3H), 2.34-2.29 (m, 1H), 2.24-2.09 |
| | (m, 3H), 2.06-1.99 (m, 1H), 1.76-1.68 (m, 1H) (3 Exchangeable |
| | protons not observed). |
| 375 | MS m/z 470.0, 472.0, 474.0 [M + H]⁺; ¹H NMR (METHANOL-d₄) δ: |
| | 7.33 (d, J = 5.0 Hz, 1H), 7.07-7.14 (m, 1H), 6.96-7.04 (m, 1H), 6.67 (s, |
| | 1H), 4.79 (s, 2H), 3.46-3.62 (m, 2H), 2.67 (s, 3H), 2.33-2.43 (m, 1H), |
| | 2.12-2.21 (m, 1H), 2.02-2.12 (m, 1H), 1.88-1.99 (m, 1H), 1.73-1.86 (m, |
| | 1H), 1.48-1.65 (m, 3H). (4 exchangeable protons not observed). |

### Example 11

### Preparation of Compound 139

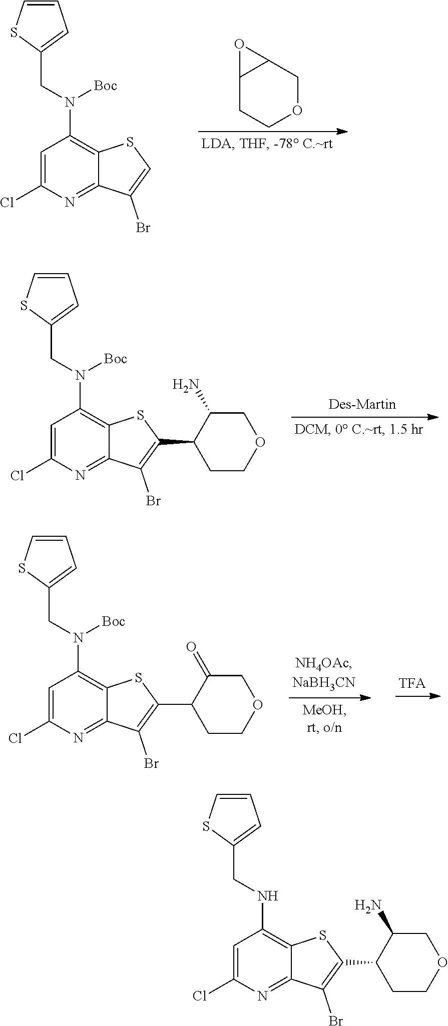 [SMILES: CC(C)(C)OC(=O)N(Cc1cccs1)c1cc(Cl)nc2c(Br)csc12 ; C1CC2OC2CO1 ; CC(C)(C)OC(=O)N(Cc1cccs1)c1cc(Cl)nc2c(Br)c([C@@H]3CCOC[C@H]3N)sc12 ; CC(C)(C)OC(=O)N(Cc1cccs1)c1cc(Cl)nc2c(Br)c(C3CCOCC3=O)sc12 ; N[C@H]1COCC[C@@H]1c1sc2c(NCc3cccs3)cc(Cl)nc2c1Br]

### Step 1: trans-tert-Butyl (3-bromo-5-chloro-2-((3S,4R)-3-hydroxytetrahydro-2H-pyran-4-yl)thieno[3,2-b]pyridin-7-yl)(thiophen-2-ylmethyl)carbamate

A solution of tert-butyl N-(3-bromo-5-chloro-thieno[3,2-b]pyridin-7-yl)-N-(2-thienylmethyl)carbamate (1.38 g, 3.0 mmol) and 3,7-dioxabicyclo[4.1.0]heptane (901 mg, 9.0 mmol) in THF (20 mL, 246 mmol, 100 mass %) was cooled at −78° C. under argon, followed by the addition of LDA in THF/hexanes (3.9 mL, 3.9 mmol, 1.0 mol/L) dropwise. The mixture was stirred at −78° C. for 1 hr and the cooling bath was removed and stirred for an additional hour at RT, quenched with NH₄Cl (aq.) and stirred for 0.5 hr, extracted with ethyl acetate and dried. This was purified on a silica column, eluted with ethyl acetate in hexanes (0˜50%), to provide tert-butyl (3-bromo-5-chloro-2-(3-hydroxytetrahydro-2H-pyran-4-yl)thieno[3,2-b]pyridin-7-yl)(thiophen-2-ylmethyl)carbamate (824 mg 49% Yield). MS m/z 559.9 [M+H]⁺, ¹H NMR (CHLOROFORM-d) δ: 7.25 (dd, J=5.0, 1.0 Hz, 1H), 7.09 (s, 1H), 6.89 (dd, J=5.0, 3.5 Hz, 1H), 6.80 (d, J=3.3 Hz, 1H), 4.97-5.08 (m, 2H), 4.09-4.20 (m, 2H), 3.99-4.07 (m, 1H), 3.84-3.95 (m, 1H), 3.44-3.60 (m, 1H), 3.25-3.37 (m, 1H), 2.00-2.04 (m, 1H), 1.81-1.96 (m, 1H), 1.41-1.58 (m, 9H).

### Step 2: tert-Butyl N-[3-bromo-5-chloro-2-(3-oxotetrahydropyran-4-yl)thieno[3,2-b]pyridin-7-yl]-N-(2-thienylmethyl)carbamate

To a solution of tert-butyl (3-bromo-5-chloro-2-(3-hydroxytetrahydro-2H-pyran-4-yl)thieno[3,2-b]pyridin-7-yl)(thiophen-2-ylmethyl)carbamate (824 mg, 1.47 mmol), at 0° C. was added Dess-Martin periodinane (965 mg, 2.20 mmol). The cooling bath was removed after the addition, and the reaction mixture was stirred at rt for 1.5 hr. LC-MS showed a complete conversion achieved. This was diluted with dichloromethane and washed with sodium thiosulfate, Na₂CO₃ (aq.), dried and filtered to remove drying agent. The filtrate was evaporated to dryness and the residue was chromatographed (ethyl acetate in hexanes, 0˜60%) to provide tert-butyl N-[3-bromo-5-chloro-2-(3-oxotetrahydropyran-4-yl)thieno[3,2-b]pyridin-7-yl]-N-(2-thienylmethyl)carbamate (799 mg, 97% Yield). MS n z 558.7 [M+H]⁺.

### Step 3: trans-2(-3-Aminotetrahydropyran-4-yl)-3-bromo-5-chloro-N-(2-thienylmethyl)thieno[3,2-b]pyridin-7-amine;2,2,2-trifluoroacetic acid

A mixture of tert-butyl N-[3-bromo-5-chloro-2-(3-oxotetrahydropyran-4-yl)thieno[3,2-b]pyridin-7-yl]-N-(2-thienylmethyl)carbamate (400 mg, 0.72 mmol) and ammonium acetate (553 mg, 7.17 mmol) in methanol (1.5 mL) was stirred at rt for 30 min, followed by the addition of NaBH₃CN (47 mg, 0.72 mmol). The mixture was stirred at rt overnight, and diluted with ethyl acetate and water and separated. Aqueous layer was extracted with ethyl acetate. The combined organics was washed with water and brine, dried and concentrated. The residue was chromatographed (ethyl acetate in hexanes, 0˜100%) to provide tert-butyl N-[2-[(3R,4S)-3-aminotetrahydropyran-4-yl]-3-bromo-5-chloro-thieno[3,2-b]pyridin-7-yl]-N-(2-thienylmethyl)carbamate. This was dissolved in dichloromethane (2 mL) and treated with trifluoroacetic acid (2 mL) at rt for 1.5 hr and evaporated. The residue was purified on a prep C18 column (Sunfire C18), eluted with 0.1% TFA modified acetonitrile in water (0˜100%) to provide trans-2-(3-aminotetrahydropyran-4-yl)-3-bromo-5-chloro-N-(2-thienylmethyl)thieno[3,2-b]pyridin-7-amine;2,2,2-trifluoroacetic acid (75 mg, 18% Yield). MS m/z 460.0 [M+H]⁺, ¹H NMR (METHANOL-d₄) δ 7.27-7.34 (m, 1H), 7.05-7.12 (m, 1H), 6.95-7.02 (m, 1H), 6.60-6.68 (m, 1H), 4.76-4.81 (m, 2H), 4.18-4.26 (m, 1H), 4.06-4.15 (m, 1H), 3.68-3.79 (m, 1H), 3.55-3.67 (m, 2H), 3.47-3.55 (m, 1H), 2.04-2.16 (m, 2H). trifluoracetic acid salt, (4 exchangeable protons are not seen).

The compounds below were prepared according to the procedure of Example 11 by substituting the appropriate starting materials, reagents, and reaction conditions, and followed by chiral SFC resolution if needed.

| Compound | Spectral Data |
|---|---|
| 140 | MS m/z 460.0 [M + H]⁺; ¹H NMR (METHANOL-d₄) δ: 7.27-7.34 (m, |
| | 1H), 7.06-7.13 (m, 1H), 6.95-7.02 (m, 1H), 6.64 (s, 1H), 4.75-4.81 (m, |
| | 2H), 4.18-4.26 (m, 1H), 4.07-4.15 (m, 1H), 3.68-3.78 (m, 1H), 3.55- |
| | 3.67 (m, 2H), 3.45-3.55 (m, 1H), 2.04-2.18 (m, 2H). trifluoracetic acid |
| | salt, (3 exchangeable protons are not seen) |
| 238 | MS m/z 444.0 [M + H]⁺; ¹H NMR (METHANOL-d₄) δ: 7.47 (s, 1H), |
| | 6.72 (s, 1H), 6.34-6.42 (m, 2H), 4.58 (s, 2H), 4.20-4.27 (m, 1H), 4.08- |
| | 4.15 (m, 1H), 3.49-3.79 (m, 4H), 2.07-2.17 (m, 2H). (3 exchangeable |
| | protons are not seen) |
| 245 | MS m/z 491.0 [M + H]⁺; ¹H NMR (METHANOL-d₄) δ: 7.44 (s, 1H), |
| | 6.37-6.30 (m, 2H), 4.77 (s, 2H), 4.10-3.95 (m, 2H), 3.62-3.51 (m, |
| | 1H), 3.45 (td, J = 11.2, 4.0 Hz, 1H), 3.30-3.22 (m, 1H), 3.18 (td, J = |
| | 10.5, 4.0 Hz, 1H), 2.05-1.95 (m, 1H), 1.92-1.79 (m, 1H). (3 |
| | Exchangeable protons not observed). |
| 272 | MS m/z 414.1 [M + H]⁺; ¹H NMR (METHANOL-d₄) δ: 7.30-7.37 (m, |
| | 1H), 7.09-7.13 (m, 1H), 6.97-7.03 (m, 1H), 6.67 (s, 1H), 4.80 (s, 2H), |
| | 4.20-4.27 (m, 1H), 4.09-4.16 (m, 1H), 3.52-3.76 (m, 4H), 2.09-2.17 (m, |
| | 2H). (3 exchangeable protons are not seen) |
| 275 | MS m/z 506.0 [M + H]⁺; ¹H NMR (METHANOL-d₄) δ: 8.35 (s, 1H, |
| | formic acid), 7.33-7.27 (m, 1H), 7.11-7.06 (m, 1H), 6.98 (dd, J = 5.1, |
| | 3.4 Hz, 1H), 6.60 (s, 1H), 4.76 (s, 2H), 4.18 (dd, J = 10.9, 4.2 Hz, 1H), |
| | 4.10-4.02 (m, 1H), 3.70-3.49 (m, 3H), 3.44 (dd, J = 12.3, 8.6 Hz, |
| | 1H), 2.10-1.93 (m, 2H). (3 Exchangeable protons not observed). |
| 314 | MS m/z 398.0 [M + H]+; 1H NMR (METHANOL-d4) δ: 7.47 (d, J = 1.1 |
| | Hz, 1H), 6.73 (s, 1H), 6.32-6.41 (m, 2H), 4.58 (s, 2H), 4.23 (dd, J = 10.4, |
| | 3.6 Hz, 1H), 4.12 (br d, J = 11.5 Hz, 1H), 3.47-3.76 (m, 4H), 2.07-2.18 |
| | (m, 2H). (3 exchangeable protons are not seen) |
| 315 | MS m/z 414.1 [M + H]⁺; ¹H NMR (METHANOL-d₄) δ: 7.30-7.37 (m, |
| | 1H), 7.09-7.13 (m, 1H), 6.97-7.03 (m, 1H), 6.67 (s, 1H), 4.80 (s, 2H), |
| | 4.20-4.27 (m, 1H), 4.09-4.16 (m, 1H), 3.52-3.76 (m, 4H), 2.09-2.17 (m, |
| | 2H). (3 exchangeable protons are not seen) |
| 316 | MS m/z 444.0 [M + H]⁺; ¹H NMR (METHANOL-d₄) δ: 7.47 (s, 1H), |
| | 6.72 (s, 1H), 6.34-6.42 (m, 2H), 4.58 (s, 2H), 4.20-4.27 (m, 1H), 4.08- |
| | 4.15 (m, 1H), 3.49-3.79 (m, 4H), 2.07-2.17 (m, 2H). (3 exchangeable |
| | protons are not seen) |
| 319 | MS m/z 398.0 [M + H]⁺; ¹H NMR (METHANOL-d₄) δ: 7.47 (d, J = 1.1 |
| | Hz, 1H), 6.73 (s, 1H), 6.32-6.41 (m, 2H), 4.58 (s, 2H), 4.23 (dd, J = 10.4, |
| | 3.6 Hz, 1H), 4.12 (br d, J = 11.5 Hz, 1H), 3.47-3.76 (m, 4H), 2.07-2.18 |
| | (m, 2H)., (3 exchangeable protons are not seen) |
| 328 | MS m/z 454.0 [M + H]⁺; ¹H NMR (METHANOL-d₄) δ: 7.38 (s, 5H), |
| | 6.49 (s, 1H), 4.61 (s, 2H), 4.20-4.29 (m, 1H), 4.08-4.17 (m, 1H), 3.70- |
| | 3.80 (m, 1H), 3.58-3.69 (m, 2H), 3.49-3.58 (m, 1H), 2.08-2.19 (m, 2H). |
| | (3 exchangeable protons are not seen) |
| 339 | MS m/z 445.0 [M + H]⁺; ¹H NMR (METHANOL-d₄) δ: 7.43 (s, 1H), |
| | 6.37-6.32 (m, 2H), 4.76 (s, 2H), 4.03 (td, J = 11.9, 4.3 Hz, 2H), 3.54 |
| | (t, J = 11.7 Hz, 1H), 3.42 (td, J = 11.1, 4.1 Hz, 1H), 3.24 (t, J = 10.7 Hz, |
| | 1H), 3.07 (td, J = 10.3, 4.6 Hz, 1H), 2.02-1.95 (m, 1H), 1.94-1.81 (m, |
| | 1H). (3 Exchangeable protons not observed). |
| 347 | MS m/z 461.0 [M + H]⁺; ¹H NMR (DMSO-d₆) δ: 9.07 (t, J = 5.9 Hz, 1H), |
| | 7.34 (d, J = 5.1 Hz, 1H), 7.01 (d, J = 3.5 Hz, 1H), 6.91 (dd, J = 5.1, 3.5 |
| | Hz, 1H), 4.84-4.69 (m, 2H), 3.90-3.75 (m, 2H), 3.34 (t, J = 11.6 Hz, |
| | 1H), 3.13 (td, J = 11.2, 3.9 Hz, 1H), 3.00 (t, J = 10.6 Hz, 1H), 2.76 (td, J |
| | = 10.1, 4.5 Hz, 1H), 1.82 (d, J = 13.8 Hz, 1H), 1.65-1.50 (m, 1H). (2 |
| | Exchangeable protons not observed). |
| 354 | MS m/z 399.0 [M + H]⁺; ¹H NMR (DMSO-d₆) δ: 9.05 (t, J = 5.5 Hz, 1H), |
| | 8.36 (s, 0.2 H, formic acid), 7.61 (d, J = 1.8 Hz, 1H), 6.42 (dd, J = 3.3, |
| | 1.8 Hz, 1H), 6.35 (d, J = 3.3 Hz, 1H), 4.75-4.60 (m, 2H), 4.00-3.84 |
| | (m, 2H), 3.50-3.35 (m, 1H), 3.23-3.14 (m, 1H), 3.07 (t, J = 10.5 Hz, |
| | 1H), 2.79 (td, J = 10.1, 4.5 Hz, 1H), 1.88 (dd, J = 13.3, 3.9 Hz, 1H), |
| | 1.75-1.60 (m, 1H). |
| 381 | MS m/z 454.0 [M + H]⁺; ¹H NMR (METHANOL-d₄) δ: 7.38 (s, 5H), |
| | 6.49 (s, 1H), 4.61 (s, 2H), 4.20-4.29 (m, 1H), 4.08-4.17 (m, 1H), 3.70- |
| | 3.80 (m, 1H), 3.58-3.69 (m, 2H), 3.49-3.58 (m, 1H), 2.08-2.19 (m, 2H). |
| | (3 exchangeable protons are not seen) |
| 388 | MS m/z 408.1 [M + H]⁺; ¹H NMR (METHANOL-d₄) δ: 7.38 (s, 4H), |
| | 7.27-7.32 (m, 1H), 6.49 (s, 1H), 4.61 (s, 2H), 4.21-4.27 (m, 1H), 4.09- |
| | 4.16 (m, 1H), 3.49-3.77 (m, 4H), 2.09-2.17 (m, 2H). (3 exchangeable |
| | protons are not seen) |
| 410 | MS m/z 398.0 [M + H]⁺; ¹H NMR (METHANOL-d₄) δ: 7.47 (d, J = 1.1 |
| | Hz, 1H), 6.73 (s, 1H), 6.32-6.41 (m, 2H), 4.58 (s, 2H), 4.23 (dd, J = 10.4, |
| | 3.6 Hz, 1H), 4.12 (br d, J = 11.5 Hz, 1H), 3.47-3.76 (m, 4H), 2.07-2.18 |
| | (m, 2H)., (3 exchangeable protons are not seen) |

### Example 12

### Preparation of Compound 146

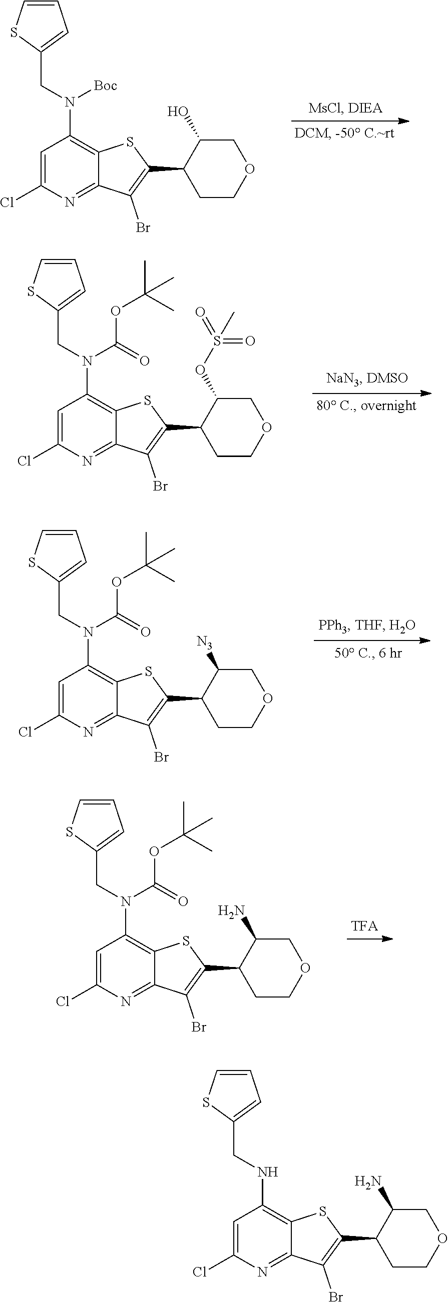

### Step 1: trans-4-(3-Bromo-7-((tert-butoxycarbonyl)(thiophen-2-ylmethyl)amino)-5-chlorothieno[3,2-b]pyridin-2-yl)tetrahydro-2H-pyran-3-yl methanesulfonate

To a solution of tert-butyl N-[3-bromo-5-chloro-2-(3-hydroxytetrahydropyran-4-yl)thieno[3,2-b]pyridin-7-yl]-N-(2-thienylmethyl)carbamate (162 mg, 0.29 mmol), prepared in Example 11, N,N-diisopropyamine (101 μL, 0.58 mmol) in dichloromethane (5 mL), at −50° C., was added methanesulfonyl chloride (27 μL, 0.35 mmol) and stirred for 0.5 hr, then warmed up to rt and stirred for an additional 0.5 hr. LC/MS showed a clean conversion achieved. The reaction was quenched by NaHCO₃ (aq.) and the organic was separated and dried, evaporated to dryness, used directly. MS m/z 639.1 [M+H]⁺.

### Step 2: cis-tert-Butyl N-[2-(3-azidotetrahydropyran-4-yl)-3-bromo-5-chloro-thieno[3,2-b]pyridin-7-yl]-N-(2-thienylmethyl)carbamate

The material prepared in Step 1, was treated with sodium azide (188 mg, 2.90 mmol) in DMSO (1.0 mL) at 80° C. overnight, cooled, diluted with ethyl acetate, washed with water and brine, dried over anhydrous MgSO₄, and filtered. The solvent was removed and the residue was chromatographed (ethyl acetate in hexanes, 0˜70%) to provide tert-butyl N-[2-(3-azidotetrahydropyran-4-yl)-3-bromo-5-chloro-thieno[3,2-b]pyridin-7-yl]-N-(2-thienylmethyl)carbamate (24 mg, 14% Yield). MS m/z 585.5 [M+H]⁺.

### Step 3: cis-tert-Butyl (2-((3R,4R)-3-aminotetrahydro-2H-pyran-4-yl)-3-bromo-5-chlorothieno[3,2-b]pyridin-7-yl)(thiophen-2-ylmethyl)carbamate

To a solution of cis-tert-butyl N-[2-(3-azidotetrahydropyran-4-yl)-3-bromo-5-chloro-thieno[3,2-b]pyridin-7-yl]-N-(2-thienylmethyl)carbamate (24 mg, 0.041 mmol) in tetrahydrofuran (1 mL, 12.3 mmol, 100 mass %) and water (0.25 mL, 14 mmol) was added triphenylphospine (12 mg, 0.045 mmol). The mixture was stirred at 50° C. for 6 hr and evaporated. The crude residue was used without further purification. MS m/z 559.6 [M+H]⁺.

### Step 4: 2-(3-Aminotetrahydropyran-4-yl)-3-bromo-5-chloro-N-(2-thienylmethyl)thieno[3,2-b]pyridin-7-amine;formic acid

The above material was treated with dichloromethane (0.5 mL) and trifluoroacetic acid (1 mL) at rt for 3 hr and evaporated to dryness. The residue was purified by on a Sunfire column using 0.1% formic acid modified acetonitrile/H₂O (0˜100%) to provide 2-(3-aminotetrahydropyran-4-yl)-3-bromo-5-chloro-N-(2-thienylmethyl)thieno[3,2-b]pyridin-7-amine;formic acid (14 mg, 62% Yield, 2 steps) as a white powder. MS m/z 459.7 [M+H]⁺, 1H NMR (METHANOL-d₄) δ 8.19-8.43 (m, 1H), 7.30 (d, J=5.0 Hz, 1H), 7.05-7.13 (m, 1H), 6.94-7.03 (m, 1H), 6.61 (s, 1H), 4.77 (s, 2H), 4.13-4.23 (m, 1H), 3.97-4.13 (m, 2H), 3.85-3.97 (m, 2H), 3.64-3.79 (m, 1H), 2.41-2.58 (m, 1H), 1.92-2.08 (m, 1H). formic acid salt, (4 exchangeable protons are not seen).

### Example 13

### Preparation of Compound 127

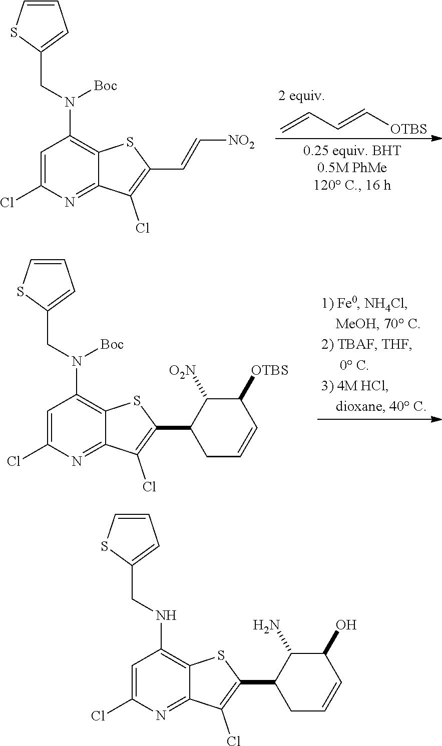

### Step 1: tert-Butyl (2-((1R/S,5S/R,6S/R)-5-((tert-butyldimethylsilyl)oxy)-6-nitrocyclohex-3-en-1-yl)-3,5-dichlorothieno[3,2-b]pyridin-7-yl)(thiophen-2-ylmethyl)carbamate

To a solution of tert-butyl (E)-(3,5-dichloro-2-(2-nitrovinyl)thieno[3,2-b]pyridin-7-yl)(thiophen-2-ylmethyl)carbamate (1.04 g, 2.15 mmol, 1.0 eq.) and in toluene (4.3 mL) was added butylated hydroxytoluene (BHT, 118 mg, 0.53 mmol, 0.25 eq.) followed by (E)-(buta-1,3-dien-1-yloxy)(tert-butyl)dimethylsilane (792 mg, 4.3 mmol, 2.0 eq.). The mixture was carefully sparged with nitrogen and sealed before stirring, protected from the light, at 120° C. for 24 h. The crude reaction mixture was crudely purified through a short silica gel column with ethyl acetate and hexanes (5 to 30% gradient) to yield a 2:2:1 mixture (937 mg, 65% yield) of tert-butyl (2-((1R/S,5S/R,6S/R)-5-((tert-butyldimethylsilyl)oxy)-6-nitrocyclohex-3-en-1-yl)-3,5-dichlorothieno[3,2-b]pyridin-7-yl)(thiophen-2-ylmethyl)carbamate:tert-butyl (2-((1R/S,5R/S,6S/R)-5-((tert-butyldimethylsilyl)oxy)-6-nitrocyclohex-3-en-1-yl)-3,5-dichlorothieno[3,2-b]pyridin-7-yl)(thiophen-2-ylmethyl)carbamate:tert-butyl (2-((1S/R,2S/R,6R/S)-2-((tert-butyldimethylsilyl)oxy)-6-nitrocyclohex-3-en-1-yl)-3,5-dichlorothieno[3,2-b]pyridin-7-yl)(thiophen-2-ylmethyl)carbamate. From which the desired regio- and diastereomer, tert-butyl (2-((1R/S,5S/R,6S/R)-5-((tert-butyldimethylsilyl)oxy)-6-nitrocyclohex-3-en-1-yl)-3,5-dichlorothieno[3,2-b]pyridin-7-yl)(thiophen-2-ylmethyl)carbamate (370 mg) was isolated after C18 chromatography with 0.1% formic acid modified acetonitrile in water (30 to 100% gradient). MS m/z 692.1 [M+Na]^{+ 1}H NMR (400 MHz, CHLOROFORM-d) δ: 7.21 (dd, J=5.2, 1.3 Hz, 1H), 7.08 (s, 1H), 6.86 (dd, J=5.1, 3.5 Hz, 1H), 6.77 (d, J=3.5 Hz, 1H), 5.83-5.73 (m, 1H), 5.65 (d, J=10.3 Hz, 1H), 5.08-4.82 (m, 4H), 4.20 (td, J=11.3, 6.0 Hz, 1H), 2.64-2.42 (m, 2H), 1.43 (s, 9H), 0.85 (s, 9H), 0.08 (s, 3H), 0.00 (s, 3H)

### Step 2: (1S/R,5R/S,6S/R)-6-Amino-5-(3,5-dichloro-7-((thiophen-2-ylmethyl)amino)thieno[3,2-b]pyridin-2-yl)cyclohex-2-en-1-ol

To a solution of tert-butyl (3-chloro-2-((1R/S,5S/R,6S/R)-5-((tert-butyldimethylsilyl)oxy)-6-nitrocyclohex-3-en-1-yl)-5-chlorothieno[3,2-b]pyridin-7-yl)(thiophen-2-ylmethyl)carbamate (94 mg, 0.14 mmol, 1.0 eq.) in methanol (1.4 mL) was added iron dust (78 mg, 1.4 mmol, 10 eq.) and ammonium chloride (374 mg, 7.0 mmol, 50 eq.) and the resulting suspension was stirred at 70° C. for 16 h. The suspension was neutralized with aqueous sodium bicarbonate filtered over Celite and the Celite was washed with ethyl acetate. The resulting bilayer was extracted with ethyl acetate and the combined extracts were washed with saturated sodium bicarbonate and brine. The organic layer was dried over MgSO₄, filtered and concentrated in vacuo to provide a residue (87 mg) that was used without further purification. The residue was dissolved in THF (1.1 mL) cooled to 0° C. and a solution of tetrabutylammonium fluoride (1.0 M, 0.14 mL) was added dropwise. The mixture was stirred at 0° C. for a further 20 minutes before being quenched with aqueous sodium bicarbonate and extracted with ethyl acetate. The combined organic extracts were washed with brine, dried over MgSO₄, filtered and concentrated in vacuo to yield a residue (48 mg) that was used without further purification. The residue was dissolved in 4M hydrochloric acid in dioxane (1 mL) and the solution was heated to 40° C. for 40 min. At which point the reaction mixture was concentrated in vacuo and purified by C18 chromatography with 0.1% formic acid modified acetonitrile in water (30 to 100% gradient) to yield (1S/R,5R/S,6S/R)-6-amino-5-(3,5-dichloro-7-((thiophen-2-ylmethyl)amino)thieno[3,2-b]pyridin-2-yl)cyclohex-2-en-1-ol (30 mg, 50% yield). MS m/z 425.9 [M+H]^{+ 1}H NMR (METHANOL-d₄) δ: 7.21 (d, J=5.1 Hz, 1H), 6.98 (s, 1H), 6.88 (t, J=4.2 Hz, 1H), 6.51 (s, 1H), 5.71 (d, J=11.0 Hz, 1H), 5.62 (d, J=10.5 Hz, 1H), 4.66 (s, 2H), 4.07 (d, J=8.2 Hz, 1H), 3.54 (q, J=9.5 Hz, 1H), 3.12 (t, J=10.1 Hz, 1H), 2.40 (s, 2H) (4 exchangeable protons not observed).

The compound below was prepared according to the procedure of Example 13 by substituting the appropriate starting materials, reagents, reaction conditions, and followed by chiral SFC resolution if needed.

| Compound | Spectral Data |
|---|---|
| 144 | MS m/z 456.0 [M + H]⁺; ¹H NMR (400 MHz, METHANOL-d₄) δ: 7.32 |
| | (dd, J = 5.1, 1.2 Hz, 1H), 7.10 (dd, J = 3.4, 1.0 Hz, 1H), 7.00 (dd, J = |
| | 5.1, 3.5 Hz, 1H), 6.59 (s, 1H), 5.83-5.72 (m, 2H), 4.77 (s, 2H), 3.50 |
| | (dd, J = 10.1, 5.6 Hz, 1H), 3.31-3.24 (m, 1H), 2.59-2.47 (m, 2H), |
| | 2.46-2.34 (m, 1H), 2.18-2.05 (m, 1H). (3 exchangeable protons are |
| | not seen) |
| 153 | MS m/z 471.9 [M + H]+; 1H NMR (ACETONE-d6) δ: 8.13 (s, 1H), 7.37 |
| | (dd, J = 5.1, 1.2 Hz, 1H), 7.16 (d, J = 3.4 Hz, 1H), 7.00 (dd, J = 5.0, 3.5 |
| | Hz, 2H), 6.66 (s, 1H), 5.78 (s, 2H), 4.87 (dd, J = 6.0, 3.4 Hz, 2H), 4.28 |
| | (s, 1H), 3.95-3.80 (m, 2H), 2.52 (d, J = 17.8 Hz, 1H), 2.42-2.29 (m, |
| | 1H). Formic acid salt, (4, exchangeable protons not observed). |
| 168 | MS m/z 441.0 [M + H]⁺; ¹H NMR (400 MHz, METHANOL-d₄) δ: 7.43 |
| | (s, 1H), 6.37-6.31 (m, 2H), 5.81-5.73 (m, 2H), 4.75 (d, J = 2.4 Hz, |
| | 2H), 3.57-3.45 (m, 1H), 3.29-3.22 (m, 1H), 2.58-2.36 (m, 3H), 2.16- |
| | 2.03 (m, 1H). (3 exchangeable protons are not seen) |
| 169 | MS m/z 394.0 [M + H]⁺; ¹H NMR (400 MHz, METHANOL-d₄) δ: 7.46- |
| | 7.42 (m, 1H), 6.71 (s, 1H), 6.37-6.30 (m, 2H), 5.92-5.86 (m, 1H), 5.81- |
| | 5.75 (m, 1H), 4.55 (s, 2H), 3.80-3.71 (m, 2H), 2.70-2.56 (m, 3H), 2.42- |
| | 2.30 (m, 1H). (3 exchangeable protons are not seen) |
| 170 | MS m/z 395.0 [M + H]⁺; ¹H NMR (400 MHz, METHANOL-d₄) δ: 7.40- |
| | 7.30 (m, 1H), 6.30-6.20 (m, 2H), 5.60-5.58 (m, 2H), 4.63 (s, 2H), 3.46 |
| | (dd, J = 9.7, 5.6 Hz, 1H), 3.30 (d, J = 4.7 Hz, 1H), 2.51-2.30 (m, 3H), |
| | 2.19-1.98 (m, 1H). (3 exchangeable protons are not seen) |
| 171 | MS m/z 380.0 [M + H]⁺; ¹H NMR (METHANOL-d₄) δ: 7.46 (dd, J = 1.9, |
| | 0.8 Hz, 1H), 6.66 (d, J = 1.3 Hz, 1H), 6.38 (dd, J = 3.3, 1.9 Hz, 1H), |
| | 6.34 (d, J = 3.3 Hz, 1H), 4.56 (s, 2H), 3.69-3.58 (m, 2H), 2.42 (s, 3H). |
| | (3, exchangeable protons not observed). |
| 172 | MS m/z 487.1 [M + H]⁺; ¹H NMR (400 MHz, METHANOL-d₄) δ: 7.42 |
| | (d, J = 0.9 Hz, 1H), 6.34 (dt, J = 6.0, 2.9 Hz, 2H), 5.82-5.69 (m, 2H), |
| | 4.79-4.70 (m, 2H), 3.52 (td, J = 10.1, 5.5 Hz, 1H), 3.34 (s, 1H), 2.56- |
| | 2.44 (m, 2H), 2.34 (q, J = 6.7 Hz, 1H), 2.15-2.03 (m, 1H). (3 |
| | exchangeable protons are not seen) |
| 176 | MS m/z 502.0 [M + H]⁺; ¹H NMR (400 MHz, METHANOL-d₄) δ: 7.29 |
| | (dd, J = 5.1, 1.1 Hz, 1H), 7.07 (d, J = 2.6 Hz, 1H), 6.97 (dd, J = 5.1, 3.5 |
| | Hz, 1H), 6.56 (s, 1H), 5.78 (d, J = 12.7 Hz, 2H), 4.74 (s, 2H), 3.50 (dd, |
| | J = 10.1, 5.5 Hz, 1H), 3.36 (dd, J = 9.5, 5.4 Hz, 1H), 2.52 (d, J = 16.7 Hz, |
| | 2H), 2.42-2.32 (m, 1H), 2.05 (dd, J = 20.6, 17.8 Hz, 1H). (3 |
| | exchangeable protons are not seen) |
| 178 | MS m/z 411.0 [M + H]⁺; ¹H NMR (400 MHz, METHANOL-d₄) δ: 7.78 |
| | (d, J = 3.2 Hz, 1H), 7.54 (d, J = 3.2 Hz, 1H), 6.56 (s, 1H), 5.88-5.61 |
| | (m, 2H), 4.90 (s, 2H), 3.55-3.40 (m, 1H), 3.28-3.15 (m, 1H), 2.61- |
| | 2.35 (m, 3H), 2.20-2.00 (m, 1H). (3 exchangeable protons are not seen) |
| 180 | MS m/z 400.0 [M + H]⁺; ¹H NMR (METHANOL-d₄) δ: 7.35 (dd, J = 1.9, |
| | 0.9 Hz, 1H), 6.62 (d, J = 0.8 Hz, 1H), 6.29-6.23 (m, 2H), 4.45 (s, 2H), |
| | 3.65 (s, 2H). (3, exchangeable protons not observed). |
| 182 | MS m/z 502.9 [M + H]⁺; ¹H NMR (400 MHz, METHANOL-d₄) δ: 7.29- |
| | 7.24 (m, 1H), 7.10-7.05 (m, 1H), 6.97-6.91 (m, 1H), 5.83-5.70 (m, |
| | 2H), 4.99-4.87 (m, 2H), 3.58-3.44 (m, 1H), 3.35-3.31 (m, 1H),2.61- |
| | 2.42 (m, 2H), 2.39-2.25 (m, 1H), 2.16-2.02 (m, 1H). (3 |
| | exchangeable protons are not seen) |
| 183 | MS m/z 446.9 [M + H]⁺; ¹H NMR (METHANOL-d₄) δ: 7.33 (s, 1H), |
| | 6.25 (t, J = 3.2 Hz, 2H), 4.66 (d, J = 3.8 Hz, 2H), 3.49 (d, J = 10.0 Hz, |
| | 1H), 3.32 (d, J = 10.0 Hz, 1H). (3, exchangeable protons not observed). |
| 184 | MS m/z 374.1 [M + H]⁺; ¹H NMR (400 MHz, METHANOL-d₄) δ: 7.44 |
| | (d, J = 1.1 Hz, 1H), 6.57 (s, 1H), 6.37-6.29 (m, 2H), 5.77 (d, J = 7.3 |
| | Hz, 2H), 4.52 (s, 2H), 3.39 (ddd, J = 23.3, 21.7, 17.4 Hz, 2H), 2.58- |
| | 2.40 (m, 3H), 2.37 (s, 3H), 2.17 (s, 1H). (3 exchangeable protons are not |
| | seen) |
| 185 | MS m/z 408.1 [M − H]⁻; 1H NMR (METHANOL-d₄) δ: 8.29 (s, 1H), 7.32 |
| | (d, J = 5.2 Hz, 1H), 7.11 (d, J = 3.4 Hz, 1H), 7.00 (t, J = 4.2 Hz, 1H), |
| | 6.63 (s, 1H), 5.92 (d, J = 10.3 Hz, 1H), 5.81 (d, J = 9.9 Hz, 1H), 4.77 (s, |
| | 2H), 3.79 (d, J = 5.9 Hz, 2H), 2.67 (dd, J = 15.9, 7.7 Hz, 3H), 2.39 (d, J = |
| | 16.6 Hz, 1H). Formic acid salt, (3, exchangeable protons not |
| | observed). |
| 186 | MS m/z 456.0 [M + H]⁺; ¹H NMR (400 MHz, METHANOL-d₄) δ: 7.32 |
| | (dd, J = 5.1, 1.2 Hz, 1H), 7.10 (dd, J = 3.4, 1.0 Hz, 1H), 7.00 (dd, J = |
| | 5.1, 3.5 Hz, 1H), 6.59 (s, 1H), 5.83-5.72 (m, 2H), 4.77 (s, 2H), 3.50 |
| | (dd, J = 10.1, 5.6 Hz, 1H), 3.31-3.24 (m, 1H), 2.59-2.47 (m, 2H), |
| | 2.46-2.34 (m, 1H), 2.18-2.05 (m, 1H). (3 exchangeable protons are |
| | not seen) |
| 187 | MS m/z 381.0 [M + H]⁺; ¹H NMR (METHANOL-d₄) δ: 7.32 (s, 1H), |
| | 6.24 (dd, J = 8.8, 2.7 Hz, 2H), 4.65 (d, J = 9.1 Hz, 2H), 3.39 (d, J = 10.2 |
| | Hz, 1H), 3.32 (d, J = 10.5 Hz, 1H), 2.26 (s, 3H). (3, exchangeable |
| | protons not observed). |
| 188 | MS m/z 486.0 [M + H]⁺; ¹H NMR (400 MHz, METHANOL-d₄) δ: 7.44 |
| | (d, J = 1.1 Hz, 1H), 6.63 (s, 1H), 6.37-6.29 (m, 2H), 5.79 (d, J = 12.0 |
| | Hz, 2H), 4.52 (s, 2H), 3.59 (ddd, J = 20.7, 12.0, 5.7 Hz, 2H), 2.53 (s, |
| | 2H), 2.47 (s, 1H), 2.21 (s, 1H). (3 exchangeable protons are not seen) |
| 190 | MS m/z 496.1 [M + H]⁺; ¹H NMR (400 MHz, METHANOL-d₄) δ: 7.39- |
| | 7.30 (m, 4H), 7.26 (t, J = 6.5 Hz, 1H), 6.39 (s, 1H), 5.78 (s, 2H), 4.56 (s, |
| | 2H), 3.56 (td, J = 9.8, 5.5 Hz, 1H), 3.49-3.38 (m, 1H), 2.49 (dd, J = |
| | 44.5, 10.3 Hz, 3H), 2.18 (s, 1H). (3 exchangeable protons are not seen) |
| 191 | MS m/z 456.9 [M + H]⁺; ¹H NMR (400 MHz, METHANOL-d₄) δ: 7.27 |
| | (d, J = 4.1 Hz, 1H), 7.09 (d, J = 3.4 Hz, 1H), 6.94 (dd, J = 5.1, 3.6 Hz, |
| | 1H), 5.75 (s, 2H), 4.92 (d, J = 1.9 Hz, 2H), 3.55-3.47 (m, 1H), 3.28- |
| | 3.23 (m, 1H), 2.48 (dd, J = 34.9, 18.3 Hz, 3H), 2.10 (s, 1H). (3 |
| | exchangeable protons are not seen) |
| 193 | MS m/z 375.1 [M + H]⁺; ¹H NMR (400 MHz, METHANOL-d₄) δ: 7.42 |
| | (s, 1H), 6.33 (d, J = 7.8 Hz, 2H), 5.74 (s, 2H), 4.75-4.69 (m, 2H), 3.36- |
| | 3.32 (m, 1H), 3.19-3.09 (m, 1H), 2.54-2.43 (m, 2H), 2.39-2.35 (m, 1H), |
| | 2.34 (s, 3H), 2.14-2.04 (m, 1H). (3 exchangeable protons are not seen) |
| 194 | MS m/z 450.0 [M + H]⁺; ¹H NMR (400 MHz, METHANOL-d₄) δ: 7.39- |
| | 7.31 (m, 4H), 7.26 (t, J = 6.5 Hz, 1H), 6.41 (s, 1H), 5.77 (s, 2H), 4.57 (s, |
| | 2H), 3.52 (td, J = 10.0, 5.5 Hz, 1H), 3.31 (s, 1H), 2.58-2.36 (m, 3H), |
| | 2.12 (dd, J = 15.7, 10.5 Hz, 1H). (3 exchangeable protons are not seen) |
| 195 | MS m/z 400.0 [M + H]⁺; ¹H NMR (METHANOL-d₄) δ: 7.35 (dd, J = 1.9, |
| | 0.9 Hz, 1H), 6.62 (d, J = 0.8 Hz, 1H), 6.29-6.23 (m, 2H), 4.45 (s, 2H), |
| | 3.65 (s, 2H). (3, exchangeable protons not observed). |
| 196 | MS m/z 410.0 [M + H]⁺; ¹H NMR (400 MHz, METHANOL-d₄) δ: 7.30 |
| | (dd, J = 5.1, 1.1 Hz, 1H), 7.09 (d, J = 2.6 Hz, 1H), 6.98 (dd, J = 5.1, 3.5 |
| | Hz, 1H), 6.64 (s, 1H), 5.91 (d, J = 10.4 Hz, 1H), 5.79 (d, J = 10.2 Hz, |
| | 1H), 4.75 (d, J = 9.3 Hz, 2H), 3.76 (d, J = 4.6 Hz, 2H), 2.65 (d, J = 16.4 |
| | Hz, 3H), 2.35 (d, J = 15.3 Hz, 1H). (3 exchangeable protons are not |
| | seen) |
| 198 | MS m/z 450.9 [M + H]⁺; ¹H NMR (400 MHz, METHANOL-d₄) δ: 8.49 |
| | (dd, J = 4.6, 1.5 Hz, 2H), 7.42 (d, J = 6.1 Hz, 2H), 6.37 (s, 1H), 5.77 (s, |
| | 2H), 4.67 (s, 2H), 3.53 (td, J = 10.0, 5.5 Hz, 1H), 3.30-3.22 (m, 1H), |
| | 2.49 (dt, J = 26.4, 12.4 Hz, 3H), 2.20-2.05 (m, 1H). (3 exchangeable |
| | protons are not seen) |
| 200 | MS m/z 446.9 [M + H]⁺; ¹H NMR (METHANOL-d₄) δ: 8.44 (s, 1H), |
| | 7.33 (s, 1H), 6.25 (t, J = 3.2 Hz, 2H), 4.66 (d, J = 3.8 Hz, 2H), 3.49 (d, |
| | J = 10.0 Hz, 1H), 3.32 (d, J = 10.0 Hz, 1H). Formic acid salt, (3, |
| | exchangeable protons not observed). |
| 202 | MS m/z 458.0 [M + H]⁺; ¹H NMR (400 MHz, METHANOL-d₄) δ: 6.61 |
| | (s, 1H), 5.80-5.70 (m, 2H), 4.10-4.00 (m, 2H), 3.55-3.45 (m, 1H), 3.37- |
| | 3.34 (m, 1H), 2.55-2.49 (m, 2H), 2.45-2.29 (m, 1H), 2.19-2.05 (m, 1H), |
| | 1.85-1.75 (m, 3H). (3 exchangeable protons are not seen) |
| 203 | MS m/z 405.2 [M + H]⁺; ¹H NMR (400 MHz, METHANOL-d₄) δ: 8.51 |
| | (d, J = 6.1 Hz, 2H), 7.44 (d, J = 5.8 Hz, 2H), 6.39 (s, 1H), 5.83-5.74 |
| | (m, 2H), 4.68 (d, J = 6.9 Hz, 2H), 3.53 (td, J = 10.0, 5.6 Hz, 1H), 3.29 |
| | (dd, J = 9.7, 5.4 Hz, 1H), 2.59-2.39 (m, 3H), 2.22-2.08 (m, 1H). (3 |
| | exchangeable protons are not seen) |
| 205 | MS m/z 396.0 [M + H]⁺; ¹H NMR (METHANOL-d₄) δ: 8.57 (s, 1H), |
| | 7.31 (dt, J = 4.1, 1.9 Hz, 1H), 7.10 (d, J = 3.8 Hz, 1H), 7.00 (dd, J = 5.1, |
| | 3.3 Hz, 1H), 6.55 (d, J = 2.8 Hz, 1H), 4.77 (s, 2H), 3.49-3.36 (m, 2H), |
| | 2.40 (s, 3H). Formic acid salt, (3, exchangeable protons not observed). |
| 206 | MS m/z 440.0 [M + H]⁺; ¹H NMR (400 MHz, METHANOL-d₄) δ: 7.45 |
| | (s, 1H), 6.69 (s, 1H), 6.38-6.32 (m, 2H), 5.90 (d, J = 10.1 Hz, 1H), |
| | 5.79 (d, J = 10.0 Hz, 1H), 4.54 (s, 2H), 3.77 (d, J = 6.4 Hz, 2H), 2.61 (s, |
| | 3H), 2.32 (d, J = 15.9 Hz, 1H). (3 exchangeable protons are not seen) |
| 207 | MS m/z 403.9 [M + H]⁺; ¹H NMR (400 MHz, METHANOL-d₄) δ: 7.38- |
| | 7.31 (m, 4H), 7.27 (d, J = 6.6 Hz, 1H), 6.42 (s, 1H), 5.77 (s, 2H), 4.57 |
| | (s, 2H), 3.50 (td, J = 10.0, 5.5 Hz, 1H), 3.27 (d, J = 10.1 Hz, 1H), 2.48 |
| | (dd, J = 27.9, 13.6 Hz, 3H), 2.16 (s, 1H). (3 exchangeable protons are |
| | not seen) |
| 209 | MS m/z 502.0 [M + H]⁺; ¹H NMR (400 MHz, METHANOL-d₄) δ: 7.29 |
| | (dd, J = 5.1, 1.1 Hz, 1H), 7.07 (d, J = 2.6 Hz, 1H), 6.97 (dd, J = 5.1, 3.5 |
| | Hz, 1H), 6.56 (s, 1H), 5.78 (d, J = 12.7 Hz, 2H), 4.74 (s, 2H), 3.50 (dd, |
| | J = 10.1, 5.5 Hz, 1H), 3.36 (dd, J = 9.5, 5.4 Hz, 1H), 2.52 (d, J = 16.7 Hz, |
| | 2H), 2.42-2.32 (m, 1H), 2.05 (dd, J = 20.6, 17.8 Hz, 1H). (3 |
| | exchangeable protons are not seen) |
| 212 | MS m/z 380.0 [M + H]⁺; ¹H NMR (METHANOL-d₄) δ: 7.46 (dd, J = 1.9, |
| | 0.8 Hz, 1H), 6.66 (d, J = 1.3 Hz, 1H), 6.38 (dd, J = 3.3, 1.9 Hz, 1H), |
| | 6.34 (d, J = 3.3 Hz, 1H), 4.56 (s, 2H), 3.69-3.58 (m, 2H), 2.42 (s, 3H). |
| | (3, exchangeable protons not observed). |
| 214 | MS m/z 406.1 [M + H]⁺; ¹H NMR (400 MHz, METHANOL-d₄) δ: 8.55- |
| | 8.46 (m, 2H), 7.45-7.37 (m, 2H), 5.90-5.73 (m, 2H), 4.84-4.80(m, 2H), |
| | 3.70-3.45 (m, 2H), 2.61-2.50 (m, 2H), 2.23-2.21 (m, 1H), 1.95-1.93 |
| | (m, 1H). (3 exchangeable protons are not seen) |
| 217 | MS m/z 455.8 [M + H]⁺; ¹H NMR (400 MHz, METHANOL-d₄) δ: 7.40 |
| | (s, 1H), 7.29 (s, 1H), 7.08 (d, J, J = 4.6 Hz, 1H), 6.56 (s, 1H), 5.90 (s, |
| | 1H), 5.81 (s, 1H), 4.58 (s, 2H), 3.80 (s, 2H), 2.63 (s, 3H), 2.36 (s, 1H). |
| | (3 exchangeable protons are not seen) |
| 219 | MS m/z 458.9 [M + H]⁺; ¹H NMR (400 MHz, METHANOL-d₄) δ: 5.78- |
| | 5.75 (m, 2H), 4.30-4.26 (m, 2H), 3.57-3.49 (m, 1H), 3.38-3.32 (m, |
| | 1H), 2.58-2.46 (m, 2H), 2.41-2.30 (m, 1H), 2.18-2.05 (m, 1H), 1.81- |
| | 1.78 (m, 3H). (3 exchangeable protons are not seen) |
| 220 | MS m/z 387.9 [M − H]⁻; ¹H NMR (METHANOL-d₄) δ: 7.30 (d, J = 5.1 |
| | Hz, 1H), 7.08 (d, J = 3.5 Hz, 1H), 6.98 (dd, J = 5.7, 3.2 Hz, 1H), 6.52 (s, |
| | 1H), 5.78 (q, J = 7.3 Hz, 2H), 4.75 (s, 2H), 3.42-3.34 (m, 1H), 3.33 (s, |
| | 3H), 3.26 (td, J = 9.9, 5.3 Hz, 1H), 2.60-2.40 (m, 3H), 2.20-2.10 (m, |
| | 1H). (3, exchangeable protons not observed). |
| 221 | MS m/z 410.9 [M + H]⁺; ¹H NMR (METHANOL-d₄) δ: 7.45 (s, 1H), |
| | 6.70-6.60 (m, 1H), 6.39-6.30 (m, 2H), 4.55 (s, 2H), 3.75-3.45 (m, 1H), |
| | 3.25-3.09 (m, 1H), 2.86-2.41 (m, 1H), 2.24-1.96 (m, 2H), 1.93-1.91 |
| | (m, 1H), 1.90-1.80 (m, 1H), 1.76-1.65 (m, 1H). (3, exchangeable |
| | protons not observed). |
| 222 | MS m/z 390.1 [M + H]⁺; ¹H NMR (400 MHz, METHANOL-d₄) δ: 7.33- |
| | 7.23 (m, 1H), 7.11-7.01 (m, 1H), 7.00-6.91 (m, 1H), 6.49 (s, 1H), |
| | 5.83-5.66 (m, 2H), 4.74 (s, 2H), 3.29-3.25 (m, 1H), 3.20-3.11 (m, |
| | 1H), 2.55-2.27 (m, 6H), 2.15-2.02 (m, 1H). (3 exchangeable protons |
| | are not seen) |
| 223 | MS m/z 392.0 [M + H]⁺; ¹H NMR (400 MHz, METHANOL-d₄) δ: 7.72 |
| | (d, J = 3.4 Hz, 1H), 7.50 (d, J = 3.4 Hz, 1H), 5.88-5.73 (m, 2H), 5.1-4.98 |
| | (m, 2H), 3.54-3.47 (m, 1H), 3.46-3.37 (m, 1H), 2.61-2.53 (m, 2H), |
| | 2.49-2.42 (m, 1H), 2.37 (s, 3H), 2.29-2.16 (m, 1H). (3 exchangeable |
| | protons are not seen) |
| 228 | MS m/z 474.0 [M + H]⁺; ¹H NMR (METHANOL-d₄) δ: 7.33 (d, J = 5.2 |
| | Hz, 1H), 7.11 (d, J = 3.4 Hz, 1H), 7.00 (dd, J = 5.1, 3.5 Hz, 1H), 6.66 (s, |
| | 1H), 4.79 (s, 2H), 3.73-3.65 (m, 1H), 3.63-3.52 (m, 1H), 3.26-3.12 |
| | (m, 1H), 2.21-2.14 (m, 1H), 2.13-2.04 (m, 1H), 2.00-1.92 (m, 1H), |
| | 1.60 (q, J = 11.5 Hz, 2H), 1.37-1.23 (m, 1H). Trifluoroacetic acid salt, |
| | (5, exchangeable protons not observed). |
| 229 | MS m/z 497.1 [M + H]⁺; ¹H NMR (METHANOL-d₄) δ: 7.30 (ddd, J = |
| | 28.3, 18.3, 7.3 Hz, 5H), 5.75 (s, 2H), 4.81-4.71 (m, 2H), 3.52 (td, J = |
| | 10.0, 5.4 Hz, 1H), 3.34 (s, 1H), 2.51 (t, J = 12.6 Hz, 2H), 2.40-2.27 |
| | (m, 1H), 2.09 (dd, J = 19.4, 9.1 Hz, 1H). (3, exchangeable protons not |
| | observed). |
| 230 | MS m/z 411.8 [M + H]⁺; ¹H NMR (400 MHz, METHANOL-d₄) δ: 6.65 |
| | (d, J = 1.1 Hz, 1H), 5.78 (s, 2H), 4.09 (q, J = 2.3 Hz, 2H), 3.58-3.43 |
| | (m, 1H), 3.31-3.21 (m, 1H), 2.49 (ddd, J = 36.9, 20.8, 7.0 Hz, 3H), |
| | 2.23-1.98 (m, 1H), 1.82 (t, J = 2.4 Hz, 3H). (3 exchangeable protons |
| | are not seen) |
| 233 | MS m/z 422.4 [M + H]⁺; ¹H NMR (METHANOL-d₄) δ: 7.46-7.43 (m, |
| | 1H), 6.65-6.60 (m, 1H), 6.38-6.28 (m, 2H), 4.53 (s, 2H), 3.51-3.38 |
| | (m, 1H), 3.23-3.15 (m, 1H), 2.40-2.02 (m, 5H), 1.69 (s, 7H). (3, |
| | exchangeable protons not observed). |
| 234 | MS m/z 386.0 [M + H]⁺; ¹H NMR (400 MHz, METHANOL-d₄) δ: 8.57- |
| | 8.36 (m, 2H), 7.47-7.33 (m, 2H), 5.83-5.67 (m, 2H), 4.83-4.77 (m, |
| | 2H), 3.52-3.35 (m, 1H), 3.28-3.21 (m, 1H), 2.61-2.47 (m, 2H), 2.45- |
| | 2.30 (m, 4H), 2.22-2.07 (m, 1H). (3 exchangeable protons are not |
| | seen) |
| 236 | MS m/z 424.1 [M + H]⁺; ¹H NMR (400 MHz, METHANOL-d₄) δ: 8.56- |
| | 8.52 (m, 1H), 8.38-8.34 (m, 1H), 7.45-7.32 (m, 1H), 5.79-5.66 (m, |
| | 2H), 4.76 (s, 2H), 3.15-3.02 (m, 2H), 2.45-2.32 (m, 2H), 2.29-2.19 |
| | (m, 1H), 2.04-1.94 (m, 1H). (3 exchangeable protons are not seen) |
| 239 | MS m/z 424.1 [M + H]⁺; ¹H NMR (400 MHz, METHANOL-d₄) δ: 8.51- |
| | 8.42 (m, 1H), 8.39-8.27 (m, 1H), 7.44-7.25 (m, 1H), 6.48 (s, 1H), |
| | 5.93-5.66 (m, 2H), 4.82-4.67 (m, 2H), 3.60-3.45 (m, 1H), 3.37-3.32 |
| | (m, 1H), 2.67-2.33 (m, 3H), 2.25-2.04 (m, 1H). (3 exchangeable |
| | protons are not seen) |
| 240 | MS m/z 411.1 [M + H]⁺; ¹H NMR (400 MHz, METHANOL-d₄) δ: 7.27 |
| | (dd, J = 5.1, 1.2 Hz, 1H), 7.09 (dd, J = 3.5, 1.0 Hz, 1H), 6.94 (dd, J = |
| | 5.1, 3.5 Hz, 1H), 5.78-5.71 (m, 2H), 4.92 (s, 2H), 3.48 (td, J = 10.0, |
| | 5.5 Hz, 1H), 3.22 (td, J = 9.5, 5.4 Hz, 1H), 2.53-2.33 (m, 3H), 2.15- |
| | 2.03 (m, 1H). (3 exchangeable protons are not seen) |
| 241 | MS m/z 451.1 [M + H]⁺; ¹H NMR (400 MHz, METHANOL-d₄) δ: 7.60- |
| | 7.00 (m, 5H), 5.75 (d, J = 2.6 Hz, 2H), 4.81-4.72 (m, 2H), 3.60-3.40 |
| | (m, 1H), 3.30-3.20 (m, 1H), 2.55-2.32 (m, 3H), 2.13-2.01 (m, 1H). (3 |
| | exchangeable protons are not seen) |
| 247 | MS m/z 390.0 [M + H]⁺; ¹H NMR (400 MHz, METHANOL-d₄) δ: 7.38 |
| | (dd, J = 4.9, 3.0 Hz, 1H), 7.27 (s, 1H), 7.08 (d, J = 4.5 Hz, 1H), 6.43 (s, |
| | 1H), 5.75 (s, 2H), 4.56 (s, 2H), 3.24-3.11 (m, 2H), 2.46 (dd, J = 31.9, |
| | 15.5 Hz, 3H), 2.34 (d, J = 15.1 Hz, 3H), 2.10 (s, 1H). (3 exchangeable |
| | protons are not seen) |
| 249 | MS m/z 440.1 [M + H]⁺; ¹H NMR (METHANOL-d₄) δ: 6.63 (s, 1H), |
| | 4.15-3.99 (m, 2H), 3.58-3.42 (m, 1H), 3.26-3.17 (m, 1H), 2.42- |
| | 2.00 (m, 4H), 1.79 (t, J = 2.3 Hz, 3H), 1.73-1.64 (m, 6H). (3, |
| | exchangeable protons not observed). |
| 250 | MS m/z 366.1 [M + H]⁺; ¹H NMR (400 MHz, METHANOL-d₄) δ: 6.62 |
| | (d, J = 8.0 Hz, 1H), 5.81-5.71 (m, 2H), 4.07 (dd, J = 4.5, 2.2 Hz, 2H), |
| | 3.46 (td, J = 10.1, 5.5 Hz, 1H), 3.22 (td, J = 9.7, 5.3 Hz, 1H), 2.58-2.33 |
| | (m, 3H), 2.18-2.04 (m, 1H), 1.79 (t, J = 2.3 Hz, 3H). (3 exchangeable |
| | protons are not seen) |
| 253 | MS m/z 404.2 [M + H]⁺; ¹H NMR (400 MHz, METHANOL-d₄) δ: 8.43- |
| | 8.40 (m, 1H), 8.32-8.29 (m, 1H), 7.47-8.42 (m, 1H), 5.77-5.73 (m, 2H), |
| | 4.87 (s, 2H), 3.38-3.34 (m, 1H), 3.18-3.10 (m, 1H), 2.53-2.44 (m, 2H), |
| | 2.40-2.28 (m, 4H), 2.14-2.04 (m, 1H). (3 exchangeable protons are not |
| | seen) |
| 256 | MS m/z 456.0 [M + H]⁺; ¹H NMR (400 MHz, METHANOL-d₄) δ: 7.32 |
| | (dd, J = 5.1, 1.2 Hz, 1H), 7.10 (dd, J = 3.4, 1.0 Hz, 1H), 7.00 (dd, J = |
| | 5.1, 3.5 Hz, 1H), 6.59 (s, 1H), 5.83-5.72 (m, 2H), 4.77 (s, 2H), 3.50 |
| | (dd, J = 10.1, 5.6 Hz, 1H), 3.31-3.24 (m, 1H), 2.59-2.47 (m, 2H), |
| | 2.46-2.34 (m, 1H), 2.18-2.05 (m, 1H). (3 exchangeable protons are |
| | not seen) |
| 258 | MS m/z 383.9 [M + H]⁺; ¹H NMR (400 MHz, METHANOL-d₄) δ: 7.38- |
| | 7.29 (m, 4H), 7.25 (t, J = 6.7 Hz, 1H), 6.31 (d, J = 8.9 Hz, 1H), 5.75 (s, |
| | 2H), 4.56 (s, 2H), 3.32 (s, 1H), 3.23-3.09 (m, 1H), 2.56-2.30 (m, |
| | 6H), 2.11 (s, 1H). (3 exchangeable protons are not seen) |
| 261 | MS m/z 396.2 [M + H]⁺; ¹H NMR (METHANOL-d₄) δ: 7.20 (dd, J = 5.1, |
| | 1.4 Hz, 1H), 6.98 (d, J = 3.4 Hz, 1H), 6.88 (t, J = 4.4 Hz, 1H), 6.48 (s, |
| | 1H), 4.66 (s, 2H), 3.59-3.47 (m, 2H), 2.30 (s, 3H). (3, exchangeable |
| | protons not observed). |
| 263 | MS m/z 405.1 [M + H]⁺; ¹H NMR (400 MHz, METHANOL-d₄) δ: 7.40- |
| | 7.35 (m, 2H), 7.34-7.28 (m, 2H), 7.27-7.22 (m, 1H), 5.76-5.73 (m, |
| | 2H), 4.76 (s, 2H), 3.51-3.43 (m, 1H), 3.26-3.18 (m, 1H), 2.55-2.45 |
| | (m, 2H), 2.43-2.32 (m, 1H), 2.14-2.04 m, 1H). (3 exchangeable |
| | protons are not seen) |
| 268 | MS m/z 391.0 [M + H]⁺; ¹H NMR (400 MHz, METHANOL-d₄) δ: 7.25 |
| | (dd, J = 5.1, 1.1 Hz, 1H), 7.07 (d, J = 3.0 Hz, 1H), 6.93 (dd, J = 5.1, 3.5 |
| | Hz, 1H), 5.75 (t, J = 9.1 Hz, 2H), 4.92 (t, J = 9.1 Hz, 2H), 3.30-3.26 |
| | (m, 1H), 3.13 (td, J = 9.8, 5.3 Hz, 1H), 2.47 (dd, J = 10.0, 6.3 Hz, 2H), |
| | 2.39-2.26 (m, 4H), 2.13-2.02 (m, 1H). (3 exchangeable protons are |
| | not seen) |
| 276 | MS m/z 520.0 [M + H]⁺; ¹H NMR (400 MHz, METHANOL-d₄) δ: 7.99 |
| | (t, J = 57.6 Hz, 1H), 7.43 (s, 1H), 6.38-6.32 (m, 2H), 5.75 (s, 2H), 4.80 |
| | (s, 2H), 4.04 (s, 1H), 2.86 (s, 1H), 2.48 (d, J = 19.3 Hz, 1H), 2.36 (d, J = |
| | 16.8 Hz, 1H), 2.06 (d, J = 14.6 Hz, 1H), 1.41-1.19 (m, 1H). (3 |
| | exchangeable protons are not seen) |
| 278 | MS m/z 390.0 [M + H]⁺; ¹H NMR (METHANOL-d₄) δ: 7.40-7.33 (m, |
| | 4H), 7.31-7.25 (m, 1H), 6.45-6.42 (m, 1H), 4.60 (s, 2H), 3.70-3.59 |
| | (m, 2H), 2.42 (s, 3H). (3, exchangeable protons not observed). |
| 287 | MS m/z 468.1 [M + H]⁺; ¹H NMR (400 MHz, METHANOL-d₄) δ: 7.33 |
| | (dt, J = 13.3, 6.9 Hz, 2H), 7.12 (dd, J = 12.9, 5.7 Hz, 2H), 6.44 (d, J = |
| | 7.7 Hz, 1H), 5.77 (d, J = 12.8 Hz, 2H), 4.61 (s, 2H), 3.50 (td, J = 10.0, |
| | 5.5 Hz, 1H), 3.30-3.23 (m, 1H), 2.59-2.35 (m, 3H), 2.18-2.01 (m, |
| | 1H). (3 exchangeable protons are not seen) |
| 292 | MS m/z 390.0 [M + H]⁺; ¹H NMR (METHANOL-d₄) δ: 7.40-7.33 (m, |
| | 4H), 7.31-7.25 (m, 1H), 6.45-6.42 (m, 1H), 4.60 (s, 2H), 3.70-3.59 |
| | (m, 2H), 2.42 (s, 3H). (3, exchangeable protons not observed). |
| 294 | MS m/z 469.9 [M + H]⁺; ¹H NMR (METHANOL-d₄) δ: 7.31 (dd, J = 5.1, |
| | 1.2 Hz, 1H), 7.13-7.03 (m, 1H), 6.99 (dd, J = 5.1, 3.5 Hz, 1H), 6.59 (s, |
| | 1H), 5.72 (ddd, J = 9.9, 4.9, 2.4 Hz, 1H), 5.57 (dq, J = 9.9, 1.9 Hz, 1H), |
| | 4.77 (s, 2H), 3.55 (td, J = 10.8, 5.8 Hz, 1H), 2.85 (dd, J = 10.8, 8.9 Hz, |
| | 1H), 2.52-2.30 (m, 2H), 2.31-2.17 (m, 1H), 1.20 (d, J = 7.0 Hz, 3H). |
| | (3, exchangeable protons not observed). |
| 297 | MS m/z 381.0 [M + H]⁺; ¹H NMR (METHANOL-d₄) δ: 7.32 (s, 1H), |
| | 6.24 (dd, J = 8.8, 2.7 Hz, 2H), 4.65 (d, J = 9.1 Hz, 2H), 3.39 (d, J = 10.2 |
| | Hz, 1H), 3.32 (d, J = 10.5 Hz, 1H), 2.26 (s, 3H). (3, exchangeable |
| | protons not observed). |
| 298 | MS m/z 427.9 [M + H]⁺; ¹H NMR (METHANOL-d₄) δ: 7.33 (d, J = 5.1 |
| | Hz, 1H), 7.11 (d, J = 3.5 Hz, 1H), 7.03-6.98 (m, 1H), 6.67 (s, 1H), |
| | 4.79 (s, 2H), 3.67 (d, J = 10.2 Hz, 1H), 3.52 (s, 1H), 3.21 (s, 1H), 2.18 |
| | (d, J = 11.0 Hz, 1H), 2.09 (d, J = 13.5 Hz, 1H), 1.96 (d, J = 11.5 Hz, |
| | 1H), 1.79 (s, 1H), 1.60 (q, J = 12.0 Hz, 2H). (4, exchangeable protons |
| | not observed). |
| 299 | MS m/z 394.0 [M + H]⁺; ¹H NMR (400 MHz, METHANOL-d₄) δ: 7.46- |
| | 7.43 (m, 1H), 6.64 (s, 1H), 6.38-6.35 (m, 1H), 6.34-6.31 (m, 1H), |
| | 5.79-5.72 (m, 2H), 4.54 (s, 2H), 3.52-3.43 (m, 1H), 3.29-3.20 (m, 1H), |
| | 2.55-2.36 (m, 3H), 2.16-2.05 (m, 1H). (3 exchangeable protons are not |
| | seen) |
| 300 | MS m/z 412.0 [M + H]⁺; ¹H NMR (METHANOL-d₄) δ: 8.54 (s, 0.6H, |
| | formic acid), 6.51 (s, 1H), 5.88-5.65 (m, 3H), 5.61-5.50 (m, 1H), |
| | 3.91 (d, J = 5.6 Hz, 2H), 3.66-3.56 (m, 1H), 3.54-3.42 (m, 1H), 2.63- |
| | 2.41 (m, 3H), 2.25-2.12 (m, 1H), 1.72 (dd, J = 6.5, 1.6 Hz, 3H). (3 |
| | Exchangeable protons not observed). |
| 302 | MS m/z 368.1 [M + H]⁺; ¹H NMR (METHANOL-d₄) δ: 8.54 (s, 0.5H, |
| | formic acid), 6.52 (s, 1H), 5.90-5.69 (m, 3H), 5.61-5.50 (m, 1H), |
| | 3.91 (d, J = 5.7 Hz, 2H), 3.59 (ddd, J = 9.8, 9.8, 5.7 Hz, 1H), 3.48- |
| | 3.39 (m, 1H), 2.62-2.42 (m, 3H), 2.26-2.15 (m, 1H), 1.75-1.69 (m, |
| | 3H). (3 Exchangeable protons not observed). |
| 306 | MS m/z 346.1 [M + H]⁺; ¹H NMR (400 MHz, METHANOL-d₄) δ: 6.61 |
| | (s, 1H), 5.82-5.69 (m, 2H), 4.10-4.02 (m, 2H), 3.32-3.27 (m, |
| | 1Hii), 3.21-3.12 (m, 1H), 2.55-2.24 (m, 6H), 2.19-2.03 (m, 1H), |
| | 1.81-1.75 (m, 3H). (3 exchangeable protons are not seen) |
| 310 | MS m/z 440.0 [M + H]⁺; ¹H NMR (400 MHz, METHANOL-d₄) δ: 7.45 |
| | (s, 1H), 6.64 (s, 1H), 6.38-6.35 (m, 1H), 6.33 (d, J = 3.2 Hz, 1H), 5.76 |
| | (s, 2H), 4.54 (s, 2H), 3.49 (dt, J = 10.2, 5.1 Hz, 1H), 3.28-3.20 (m, |
| | 1H), 2.54-2.46 (m, 2H), 2.43-2.34 (m, 1H), 2.12 (d, J = 9.6 Hz, 1H). |
| | (3 exchangeable protons are not seen) |
| 322 | MS m/z 392.0 [M + H]⁺; ¹H NMR (METHANOL-d₄) δ: 7.29-7.20 (m, |
| | 4H), 7.20-7.11 (m, 1H), 6.32 (dd, J = 4.5, 2.4 Hz, 1H), 3.59-3.45 (m, |
| | 2H), 2.30 (s, 3H). (3, exchangeable protons not observed). |
| 323 | MS m/z 487.1 [M + H]⁺; ¹H NMR (400 MHz, METHANOL-d₄) δ: 7.42 |
| | (d, J = 0.8 Hz, 1H), 6.34 (dt, J = 6.1, 3.2 Hz, 2H), 5.79-5.71 (m, 2H), |
| | 4.79-4.69 (m, 2H), 3.52 (td, J = 10.0, 5.4 Hz, 1H), 3.34 (d, J = 5.4 Hz, |
| | 1H), 2.56-2.45 (m, 2H), 2.39-2.29 (m, 1H), 2.15-2.03 (m, 1H). (3 |
| | exchangeable protons are not seen) |
| 325 | MS m/z 535.0 [M + H]⁺; ¹H NMR (400 MHz, METHANOL-d₄) δ: 7.89 |
| | (t, J = 58.1 Hz, 1H), 7.24 (d, J = 5.1 Hz, 1H), 7.01 (s, 1H), 6.90 (dd, J = |
| | 5.0, 3.6 Hz, 1H), 6.59 (s, 1H), 5.65 (s, 2H), 4.66 (s, 2H), 3.95 (s, 1H), |
| | 2.74 (s, 1H), 2.33 (dd, J = 45.7, 18.2 Hz, 2H), 2.03-1.86 (m, 1H). (3 |
| | exchangeable protons are not seen) |
| 326 | MS m/z 385.1 [M + H]⁺; ¹H NMR (400 MHz, METHANOL-d₄) δ: 7.37 |
| | (d, J = 7.3 Hz, 2H), 7.31 (t, J = 7.4 Hz, 2H), 7.24 (t, J = 7.2 Hz, 1H), |
| | 5.76 (s, 2H), 4.81-4.70 (m, 2H), 3.65 (s, 1H), 3.45-3.36 (m, 1H), |
| | 2.52 (d, J = 20.1 Hz, 2H), 2.38 (d, J = 27.5 Hz, 4H), 2.18 (s, 1H). (3 |
| | exchangeable protons are not seen) |
| 327 | MS m/z 486.0 [M + H]⁺; ¹H NMR (400 MHz, METHANOL-d₄) δ: 7.44 |
| | (s, 1H), 6.61 (s, 1H), 6.37-6.29 (m, 2H), 5.76 (s, 2H), 4.52 (s, 2H), |
| | 3.52 (d, J = 5.4 Hz, 1H), 3.36 (d, J = 5.5 Hz, 1H), 2.51 (d, J = 18.3 Hz, |
| | 2H), 2.41-2.29 (m, 1H), 2.15 (s, 1H). (3 exchangeable protons are not |
| | seen) |
| 329 | MS m/z 472.0 [M + H]⁺; ¹H NMR (METHANOL-d₄) δ: 7.32 (d, J = 5.1 |
| | Hz, 1H), 7.11 (d, J = 3.4 Hz, 1H), 7.00 (t, J = 4.3 Hz, 1H), 6.66 (s, 1H), |
| | 5.96 (d, J = 10.7 Hz, 1H), 5.90 (d, J = 11.1 Hz, 1H), 4.79 (s, 2H), 4.69 |
| | (d, J = 7.8 Hz, 1H), 3.94 (q, J = 8.6 Hz, 1H), 3.71 (dd, J = 10.5, 7.8 Hz, |
| | 1H), 2.66 (d, J = 17.3 Hz, 1H), 2.46-2.35 (m, 1H). Trifluoroacetic acid |
| | salt, (5, exchangeable protons not observed). |
| 332 | MS m/z 438.0 [M + H]⁺; ¹H NMR (METHANOL-d₄) δ: 8.54 (s, 0.6H, |
| | formic acid), 6.48 (d, J = 7.7 Hz, 1H), 5.92-5.72 (m, 2H), 5.58 (dd, J = |
| | 5.5, 3.4 Hz, 1H), 4.88 (s, 2H), 4.08-3.98 (m, 2H), 3.71-3.57 (m, 1H), |
| | 3.54-3.45 (m, 1H), 2.75-2.45 (m, 1H), 2.40-2.15 (m, 3H), 1.98- |
| | 1.88 (m, 2H). (3 Exchangeable protons not observed). |
| 340 | MS m/z 413.6 [M + H]⁺; ¹H NMR (400 MHz, METHANOL-d₄) δ: 7.73 |
| | (d, J = 3.6 Hz, 1H), 7.52 (d, J = 3.2 Hz, 1H), 5.83-5.65 (m, 2H), 5.07 |
| | (s, 2H), 3.57-3.45 (m, 1H), 3.27-3.17 (m, 1H), 2.59-2.31 (m, 3H), |
| | 2.17-2.04 (m, 1H). (3 exchangeable protons are not seen) |
| 341 | MS m/z 402.2 [M + H]⁺; ¹H NMR (400 MHz, METHANOL-d₄) δ: 7.32 |
| | (dt, J = 13.2, 6.9 Hz, 2H), 7.12 (t, J = 8.5 Hz, 2H), 6.37 (s, 1H), 5.75 (s, |
| | 2H), 4.61 (s, 2H), 3.49-3.32 (m, 1H), 3.23-2.91 (m, 1H), 2.64-2.33 |
| | (m, 6H), 2.17-1.99 (m, 1H). (3 exchangeable protons are not seen) |
| 345 | MS m/z 496.2 [M + H]⁺; ¹H NMR (400 MHz, METHANOL-d₄) δ: 7.38- |
| | 7.30 (m, 4H), 7.25 (ddd, J = 8.5, 4.0, 2.0 Hz, 1H), 6.37 (d, J = 7.9 Hz, |
| | 1H), 5.76 (s, 2H), 4.56 (s, 2H), 3.51 (td, J = 10.1, 5.5 Hz, 1H), 3.35 (dd, |
| | J = 9.7, 5.4 Hz, 1H), 2.58-2.29 (m, 3H), 2.16-2.04 (m, 1H). (3 |
| | exchangeable protons are not seen) |
| 348 | MS m/z 530.0 [M + H]⁺; ¹H NMR (400 MHz, METHANOL-d₄) δ: 8.00 |
| | (t, J = 57.6 Hz, 1H), 7.40 (d, J = 7.3 Hz, 2H), 7.32 (t, J = 7.4 Hz, 2H), |
| | 7.26 (d, J = 7.2 Hz, 1H), 5.77 (s, 2H), 4.81 (s, 2H), 4.16 (s, 1H), 2.88 (s, |
| | 1H), 2.52 (d, J = 15.3 Hz, 1H), 2.39 (d, J = 16.8 Hz, 1H), 2.19-1.99 |
| | (m, 1H), 1.28 (s, 1H). (3 exchangeable protons are not seen) |
| 352 | MS m/z 529.2 [M + H]⁺; ¹H NMR (400 MHz, METHANOL-d₄) δ: 8.00 |
| | (t, J = 57.9 Hz, 1H), 7.34 (dq, J = 28.4, 7.2 Hz, 5H), 6.54 (s, 1H), 5.76 |
| | (s, 2H), 4.56 (s, 2H), 4.09 (s, 1H), 3.34 (s, 1H), 2.84 (s, 1H), 2.44 (dd, |
| | J = 49.9, 15.4 Hz, 2H), 2.12-1.98 (m, 1H). (3 exchangeable protons are |
| | not seen) |
| 355 | MS m/z 474.0 [M + H]⁺; ¹H NMR (METHANOL-d₄) δ: 7.32 (d, J = 5.1 |
| | Hz, 1H), 7.11 (d, J = 3.5 Hz, 1H), 7.00 (t, J = 4.3 Hz, 1H), 6.66 (s, 1H), |
| | 4.80 (s, 2H), 3.92 (s, 1H), 3.59-3.35 (m, 2H), 2.18 (d, J = 11.0 Hz, |
| | 2H), 2.00 (s, 1H), 1.70-1.48 (m, 3H). |
| | Trifluoroacetic acid salt, (5, exchangeable protons not observed). |
| 358 | MS m/z 408.4[M + H]⁺; ¹H NMR (400 MHz, METHANOL-d₄) δ: 7.89 |
| | (t, J = 58.2 Hz, 1H), 7.43 (d, J = 0.9 Hz, 1H), 6.38-6.31 (m, 2H), 5.74 |
| | (s, 2H), 4.79 (s, 2H), 3.58-3.45 (m, 1H), 3.22-3.10 (m, 1H), 2.47 (dt, |
| | J = 35.2, 18.3 Hz, 3H), 2.28 (s, 3H), 2.11-1.99 (m, 1H). (3 |
| | exchangeable protons are not seen) |
| 362 | MS m/z 502.9 [M + H]⁺; ¹H NMR (400 MHz, METHANOL-d₄) δ: 7.27 |
| | (d,J, J = 4.4 Hz, 1H), 7.08 (d, J = 3.1 Hz, 1H), 6.94 (q, J = 5.0, 3.5 Hz, |
| | 1H), 5.82-5.70 (m, 2H), 5.00-4.86 (m, 2H), 3.59-3.43 (m, 1H), 3.38- |
| | 3.32 (m, 1H), 2.59-2.43 (m, 2H), 2.41-2.27 (m, 1H), 2.18-1.99 |
| | (m, 1H). (3 exchangeable protons are not seen) |
| 365 | MS m/z 502.0 [M + H]⁺; ¹H NMR (400 MHz, METHANOL-d₄) δ: 7.29 |
| | (d, J = 5.1 Hz, 1H), 7.07 (d, J = 3.4 Hz, 1H), 6.99-6.95 (m, 1H),6.55 |
| | (s, 1H), 5.76 (s, 2H), 4.74 (s, 2H), 3.50 (td, J = 10.2, 5.5 Hz, 1H), 3.37- |
| | 3.32 (m, 1H), 2.51 (d, J = 17.5 Hz, 2H), 2.36 (dd, J = 14.2, 10.4 Hz, 1H), |
| | 2.15-2.03 (m, 1H). (3 exchangeable protons are not seen) |
| 366 | MS m/z 430.0 [M + H]⁺; ¹H NMR (METHANOL-d₄) δ: 8.58 (s, 0.4H, |
| | formic acid), 6.52 (s, 1H), 5.88 (d, J = 10.4 Hz, 1H), 5.79 (d, J = 10.4 |
| | Hz, 1H), 4.21 (s, 2H), 3.78 (s, 3H), 3.77-3.64 (m, 2H), 2.70-2.50 (m, |
| | 3H), 2.35-2.22 (m, 1H). (3 Exchangeable protons not observed). |
| 367 | MS m/z 390.1 [M + H]⁺; ¹H NMR (400 MHz, METHANOL-d₄) δ: 7.33- |
| | 7.23 (m, 1H), 7.11-7.01 (m, 1H), 7.00-6.91 (m, 1H), 6.49 (s, 1H), |
| | 5.83-5.66 (m, 2H), 4.74 (s, 2H), 3.29-3.25 (m, 1H), 3.20-3.11 (m, |
| | 1H), 2.55-2.27 (m, 6H), 2.15-2.02 (m, 1H). (3 exchangeable protons |
| | are not seen) |
| 369 | MS m/z 400.0 [M + H]⁺; ¹H NMR (METHANOL-d₄) δ: 7.35 (dd, J = 1.9, |
| | 0.9 Hz, 1H), 6.62 (d, J = 0.8 Hz, 1H), 6.29-6.23 (m, 2H), 4.45 (s, 2H), |
| | 3.65 (s, 2H). (3, exchangeable protons not observed). |
| 372 | MS m/z 446.9 [M + H]⁺; ¹H NMR (METHANOL-d₄) δ: 7.33 (s, 1H), |
| | 6.25 (t, J = 3.2 Hz, 2H), 4.66 (d, J = 3.8 Hz, 2H), 3.49 (d, J = 10.0 Hz, |
| | 1H), 3.32 (d, J = 10.0 Hz, 1H). (3, exchangeable protons not observed). |
| 373 | MS m/z 450.8 [M + H]⁺; ¹H NMR (400 MHz, METHANOL-d₄) δ: 8.49 |
| | (dd, J = 4.6, 1.5 Hz, 2H), 7.42 (d, J = 6.1 Hz, 2H), 6.37 (s, 1H), 5.77 (s, |
| | 2H), 4.67 (s, 2H), 3.52 (tt, J = 14.2, 7.1 Hz, 1H), 3.27 (s, 1H), 2.64- |
| | 2.35 (m, 3H), 2.12 (dd, J = 16.1, 10.8 Hz, 1H). (3 exchangeable protons |
| | are not seen) |
| 374 | MS m/z 407.9 [M + H]⁺; ¹H NMR (METHANOL-d₄) δ: 7.31 (dd, J = 5.3, |
| | 1.1 Hz, 1H), 7.10 (d, J = 3.4 Hz, 1H), 6.99 (dd, J = 5.1, 3.5 Hz, 1H), |
| | 6.58 (s, 1H), 4.78 (s, 2H), 3.74-3.61 (m, 1H), 3.39 (s, 1H), 3.14 (d, J = |
| | 10.8 Hz, 1H), 2.39 (s, 3H), 2.18 (d, J = 10.7 Hz, 1H), 2.06 (d, J = 13.7 |
| | Hz, 1H), 1.94 (d, J = 11.3 Hz, 1H), 1.79-1.66 (m, 1H), 1.59 (q, J = |
| | 12.8 Hz, 2H). Trifluoroacetic acid salt, (4, exchangeable protons not |
| | observed). |
| 376 | MS m/z 424.1 [M + H]⁺; ¹H NMR (400 MHz, METHANOL-d₄) δ: 8.53- |
| | 8.40 (m, 1H), 8.39-8.26 (m, 1H), 7.46-7.29 (m, 1H), 6.46 (s, 1H), |
| | 5.85-5.67 (m, 2H), 4.80-4.64 (m, 2H), 3.58-3.41 (m, 1H), 3.27-3.17 |
| | (m, 1H), 2.63-2.29 (m, 3H), 2.25 (m, 1H). (3 exchangeable protons are |
| | not seen) |
| 377 | MS m/z [M + H]+ 425.9; ¹H NMR (METHANOL-d₄) δ: 7.21 (d, J = 5.1 |
| | Hz, 1H), 6.99 (d, J = 3.5 Hz, 1H), 6.93-6.83 (m, 1H), 6.54 (s, 1H), |
| | 5.77 (s, 2H), 4.67 (s, 2H), 4.51 (d, J = 8.7 Hz, 1H), 3.71-3.55 (m, 1H), |
| | 3.50-3.41 (m, 1H), 2.51 (d, J = 17.3 Hz, 1H), 2.22 (dd, J = 17.7, 9.5 |
| | Hz, 1H). (4, exchangeable protons not observed). |
| 378 | MS m/z 348.1 [M + H]⁺; ¹H NMR (METHANOL-d₄) δ: 6.50 (s, 1H), |
| | 5.94-5.85 (m, 1H), 5.81-5.69 (m, 2H), 5.60-5.52 (m, 1H), 3.91 (d, J = |
| | 5.5 Hz, 2H), 3.70-3.56 (m, 2H), 2.70-2.50 (m, 3H), 2.40 (s, 3H), |
| | 2.39-2.28 (m, 1H), 1.71 (d, J = 6.6 Hz, 3H). (3 Exchangeable protons |
| | not observed). |
| 379 | MS m/z 520.0 [M + H]⁺; ¹H NMR (400 MHz, METHANOL-d₄) δ: 7.99 |
| | (t, J = 57.6 Hz, 1H), 7.43 (s, 1H), 6.38-6.32 (m, 2H), 5.75 (s, 2H), 4.80 |
| | (s, 2H), 4.04 (s, 1H), 2.86 (s, 1H), 2.48 (d, J = 19.3 Hz, 1H), 2.36 (d, J = |
| | 16.8 Hz, 1H), 2.06 (d, J = 14.6 Hz, 1H), 1.41-1.19 (m, 1H). (3 |
| | exchangeable protons are not seen) |
| 380 | MS m/z 410.9 [M + H]⁺; ¹H NMR (400 MHz, METHANOL-d₄) δ: 7.78 |
| | (d, J = 3.2 Hz, 1H), 7.54 (d, J = 3.2 Hz, 1H), 6.56 (s, 1H), 5.82-5.70 |
| | (m, 2H), 4.90 (s, 2H), 3.62-3.42 (m, 1H), 3.30-3.15 (m, 1H), 2.60- |
| | 2.35 (m, 3H), 2.20-2.00 (m, 1H). (3 exchangeable protons are not seen) |
| 382 | MS m/z 374.1 [M + H]⁺; ¹H NMR (400 MHz, METHANOL-d₄) δ: 7.44 |
| | (d, J = 1.1 Hz, 1H), 6.56 (s, 1H), 6.35 (d, J = 1.8 Hz, 1H), 6.31 (s, 1H), |
| | 5.75 (s, 2H), 4.52 (s, 2H), 3.34 (d, J = 4.0 Hz, 1H), 3.20 (d, J = 19.8 Hz, |
| | 1H), 2.46 (dd, J = 32.2, 16.5 Hz, 3H), 2.36 (s, 3H), 2.17-2.05 (m, 1H). |
| | (3 exchangeable protons are not seen) |
| 385 | MS m/z 454.9 [M + H]⁺; ¹H NMR (400 MHz, METHANOL-d₄) δ: 7.28 |
| | (d, J = 5.1 Hz, 1H), 7.09 (d, J = 3.5 Hz, 1H), 6.96-6.92 (m, 1H), 5.81 |
| | (d, J = 22.5 Hz, 2H), 4.93 (d, J = 5.8 Hz, 2H), 3.72-3.56 (m, 2H), 2.56 |
| | (d, J = 15.9 Hz, 3H), 2.27 (s, 1H). (3 exchangeable protons are not seen) |
| 386 | MS m/z 458.1 [M + H]⁺; ¹H NMR (400 MHz, METHANOL-d₄) δ: 6.61 |
| | (s, 1H), 5.80-5.70 (m, 2H), 4.10-4.00 (m, 2H), 3.55-3.45 (m, 1H), 3.37- |
| | 3.34 (m, 1H), 2.55-2.49 (m, 2H), 2.45-2.29 (m, 1H), 2.19-2.05 (m, 1H), |
| | 1.85-1.75 (m, 3H). (3 exchangeable protons are not seen) |
| 396 | MS m/z 441.0 [M + H]⁺; ¹H NMR (400 MHz, METHANOL-d₄) δ: 7.43 |
| | (s, 1H), 6.37-6.31 (m, 2H), 5.81-5.73 (m, 2H), 4.75 (d, J = 2.4 Hz, |
| | 2H), 3.57-3.45 (m, 1H), 3.29-3.22 (m, 1H), 2.58-2.36 (m, 3H), 2.16- |
| | 2.03 (m, 1H). (3 exchangeable protons are not seen) |
| 397 | MS m/z 411.1 [M + H]⁺; ¹H NMR (400 MHz, METHANOL-d₄) δ: 7.27 |
| | (dd, J = 5.1, 1.2 Hz, 1H), 7.09 (dd, J = 3.5, 1.0 Hz, 1H), 6.94 (dd, J = |
| | 5.1, 3.5 Hz, 1H), 5.78-5.71 (m, 2H), 4.92 (s, 2H), 3.48 (td, J = 10.0, |
| | 5.5 Hz, 1H), 3.22 (td, J = 9.5, 5.4 Hz, 1H), 2.53-2.33 (m, 3H), 2.15- |
| | 2.03 (m, 1H). (3 exchangeable protons are not seen) |
| 399 | MS m/z 418.0 [M + H]⁺; ¹H NMR (400 MHz, METHANOL-d₄) δ: 7.90 |
| | (t, J = 58.2 Hz, 1H), 7.39 (d, J = 7.4 Hz, 2H), 7.35-7.28 (m, 2H), 7.28- |
| | 7.22 (m, 1H), 5.81-5.66 (m, 2H), 4.80 (s, 2H), 3.61-3.42 (m, 1H), |
| | 3.23-3.09 (m, 1H), 2.60-2.44 (m, 2H), 2.39 (dd, J = 17.0, 5.6 Hz, |
| | 1H), 2.28 (s, 3H), 2.13-1.97 (m, 1H). (3 exchangeable protons are not |
| | seen) |
| 402 | MS m/z 469.9 [M + H]⁺; ¹H NMR (METHANOL-d₄) δ: 7.32 (dd, J = 5.1, |
| | 1.2 Hz, 1H), 7.13-7.08 (m, 1H), 7.00 (dd, J = 5.1, 3.5 Hz, 1H), 6.63 (s, |
| | 1H), 5.81-5.73 (m, 1H), 5.67 (d, J = 10.1 Hz, 1H), 4.78 (s, 2H), 3.44 |
| | (s, 1H), 3.26-3.21 (m, 1H), 2.70 (s, 1H), 2.57 (d, J = 17.4 Hz, 1H), |
| | 2.20 (t, J = 13.9 Hz, 1H), 0.99 (d, J = 7.1 Hz, 3H). (3, exchangeable |
| | protons not observed). |
| 403 | MS m/z 448.3 [M + H]⁺; ¹H NMR (400 MHz, METHANOL-d₄) δ: 7.41- |
| | 7.31 (m, 4H), 7.27 (d, J = 6.5 Hz, 1H), 6.41 (s, 1H), 5.77 (s, 2H), 4.57 |
| | (s, 2H), 3.54 (dt, J = 10.1, 4.9 Hz, 1H), 3.36 (d, J = 9.3 Hz, 1H), 2.61- |
| | 2.37 (m, 3H), 2.17 (s, 1H). (3 exchangeable protons are not seen) |
| 404 | MS m/z 403.8 [M + H]⁺; ¹H NMR (400 MHz, METHANOL-d₄) δ: 7.38- |
| | 7.31 (m, 4H), 7.27 (d, J = 6.5 Hz, 1H), 6.41 (s, 1H), 5.76 (s, 2H), 4.57 |
| | (s, 2H), 3.48 (td, J = 10.1, 5.6 Hz, 1H), 3.29-3.20 (m, 1H), 2.57-2.32 |
| | (m, 3H), 2.10 (dd, J = 14.4, 9.6 Hz, 1H). (3 exchangeable protons are |
| | not seen) |
| 411 | MS m/z 468.1 [M + H]⁺; ¹H NMR (400 MHz, METHANOL-d₄) δ: 7.33 |
| | (dt, J = 20.5, 6.7 Hz, 2H), 7.13 (dd, J = 12.6, 6.0 Hz, 2H), 6.44 (d, J = |
| | 11.0 Hz, 1H), 5.77 (d, J = 6.4 Hz, 2H), 4.62 (s, 2H), 3.85-3.36 (m, |
| | 2H), 2.62-2.38 (m, 3H), 2.12 (dd, J = 15.4, 10.4 Hz, 1H). (3 |
| | exchangeable protons are not seen) |
| 413 | MS m/z 375.1 [M + H]⁺; ¹H NMR (METHANOL-d₄) δ: 7.42 (s, 1H), |
| | 6.36-6.30 (m, 2H), 5.82-5.72 (m, 2H), 4.77-4.71m, 2H), 3.47-3.41 |
| | (m, 1H), 3.37-3.33 (m, 1H), 2.59-2.49 (m, 2H), 2.46-2.40 (m, 1H), 2.35 |
| | (s, 3H), 2.224-2.14 (m, 1H). (3, exchangeable protons not observed). |
| 414 | MS m/z 497.1 [M + H]⁺; ¹H NMR (400 MHz, METHANOL-d₄) δ: 7.30 |
| | (ddd, J = 28.1, 18.2, 7.2 Hz, 5H), 5.75 (s, 2H), 4.80-4.71 (m, 2H), 3.51 |
| | (td, J = 10.1, 5.5 Hz, 1H), 3.33 (s, 1H), 2.57-2.43 (m, 2H), 2.39-2.28 |
| | (m, 1H), 2.09 (dd, J = 18.8, 9.3 Hz, 1H). (3 exchangeable protons are |
| | not seen) |
| 415 | MS m/z 496.9 [M + H]⁺; ¹H NMR (400 MHz, METHANOL-d₄) δ: 7.39 |
| | (d, J = 2.7 Hz, 1H), 7.28 (s, 1H), 7.09 (d, J = 4.8 Hz, 1H), 6.50 (s, 1H), |
| | 5.76 (s, 2H), 4.57 (s, 2H), 3.49 (s, 1H), 3.26 (s, 1H), 2.56-2.35 (m, |
| | 3H), 2.11 (s, 1H). (3 exchangeable protons are not seen) |
| 417 | MS m/z 437.2 [M + H]⁺; ¹H NMR (400 MHz, METHANOL-d₄) δ: 7.97 |
| | (t, J = 58.0 Hz, 1H), 7.42-7.25 (m, 5H), 6.53 (s, 1H), 5.76-5.71 (m, |
| | 2H), 4.57 (s, 2H), 3.92-3.75 (m, 1H), 3.24 (td, J = 11.2, 5.6 Hz, 1H), |
| | 2.84-2.67 (m, 1H), 2.57-2.31 (m, 2H), 2.06 (dd, J = 15.3, 10.3 Hz, |
| | 1H). (3 exchangeable protons are not seen) |
| 419 | MS m/z 390.0 [M + H]⁺; ¹H NMR (400 MHz, METHANOL-d₄) δ: 7.39 |
| | (s, 1H), 7.27 (s, 1H), 7.08 (d, J = 5.1 Hz, 1H), 6.49 (s, 1H), 5.88 (s, 1H), |
| | 5.79 (s, 1H), 4.57 (s, 2H), 3.64 (s, 2H), 2.75-2.52 (m, 3H), 2.39 (s, |
| | 3H), 2.37-2.29 (m, 1H). (3 exchangeable protons are not seen) |
| 420 | MS m/z 437.2 [M + H]⁺; ¹H NMR (400 MHz, METHANOL-d₄) δ: 7.97 |
| | (t, J = 58.0 Hz, 1H), 7.43-7.24 (m, 5H), 6.54 (s, 1H), 5.76-5.72 (m, |
| | 2H), 4.57 (s, 2H), 3.89-3.76 (m, 1H), 3.24 (td, J = 11.3, 5.5 Hz, 1H), |
| | 2.75 (t, J = 12.5 Hz, 1H), 2.55-2.30 (m, 2H), 2.06 (dd, J = 15.2, 10.2 |
| | Hz, 1H). (3 exchangeable protons are not seen) |
| 424 | MS m/z 530.0 [M + H]⁺; ¹H NMR (400 MHz, METHANOL-d₄) δ: 8.00 |
| | (t, J = 57.7 Hz, 1H), 7.39 (d, J = 7.4 Hz, 2H), 7.32 (t, J = 7.4 Hz, 2H), |
| | 7.25 (t, J = 7.3 Hz, 1H), 5.74 (s, 2H), 4.81 (s, 2H), 4.00 (s, 1H), 2.87 (s, |
| | 1H), 2.47 (d, J = 16.1 Hz, 1H), 2.35 (d, J = 17.8 Hz, 1H), 2.03 (t, J = |
| | 11.4 Hz, 1H), 1.26 (d, J = 19.4 Hz, 1H). (3 exchangeable protons are not |
| | seen) |
| 427 | MS m/z 535.0 [M + H]⁺; ¹H NMR (400 MHz, METHANOL-d₄) δ: 8.01 |
| | (t, J = 58.2 Hz, 1H), 7.36 (d, J = 5.1 Hz, 1H), 7.14 (d, J = 3.4 Hz, 1H), |
| | 7.05-6.99 (m, 1H), 6.70 (s, 1H), 5.77 (s, 2H), 4.78 (s, 2H), 4.06 (s, |
| | 1H), 2.85 (s, 1H), 2.44 (dd, J = 46.7, 18.8 Hz, 2H), 2.06 (s, 1H). (3 |
| | exchangeable protons are not seen) |
| 429 | MS m/z 411.9 [M + H]⁺; ¹H NMR (400 MHz, METHANOL-d₄) δ: 6.63 |
| | (d, J = 4.1 Hz, 1H), 5.75 (s, 2H), 4.06 (d, J = 2.2 Hz, 2H), 3.49 (td, J = |
| | 10.2, 5.2 Hz, 1H), 3.25 (dd, J = 14.8, 9.4 Hz, 1H), 2.44 (dd, J = 37.6, |
| | 10.1 Hz, 3H), 2.24-2.00 (m, 1H), 1.79 (t, J = 2.3 Hz, 3H). (3 |
| | exchangeable protons are not seen) |
| 431 | MS m/z 408.1 [M − H]⁻; ¹H NMR (METHANOL-d₄) δ: 8.29 (s, 1H), 7.32 |
| | (d, J = 5.2 Hz, 1H), 7.11 (d, J = 3.4 Hz, 1H), 7.00 (t, J = 4.2 Hz, 1H), |
| | 6.63 (s, 1H), 5.92 (d, J = 10.3 Hz, 1H), 5.81 (d, J = 9.9 Hz, 1H), 4.77 (s, |
| | 2H), 3.79 (d, J = 5.9 Hz, 2H), 2.67 (dd, J = 15.9, 7.7 Hz, 3H), 2.39 (d, J |
| | = 16.6 Hz, 1H). Formic acid salt, (3, exchangeable protons not |
| | observed). |
| 433 | MS m/z 405.2 [M + H]⁺; ¹H NMR (400 MHz, METHANOL-d₄) δ: 8.49 |
| | (d, J = 6.0 Hz, 2H), 7.42 (d, J = 5.7 Hz, 2H), 6.38 (s, 1H), 5.77 (s, 2H), |
| | 4.67 (s, 2H), 3.48 (m, 1H), 3.25 m, 1H), 2.44 (m, 3H), 2.09 (m, 1H). (3 |
| | exchangeable protons are not seen) |
| 434 | MS m/z 451.1 [M + H]⁺; ¹H NMR (400 MHz, METHANOL-d₄) δ: 7.30 |
| | (ddd, J = 28.1, 18.1, 7.4 Hz, 5H), 5.77 (d, J = 14.9 Hz, 2H), 4.82-4.70 |
| | (m, 2H), 3.55-3.44 (m, 1H), 3.25 (dd, J = 9.4, 5.5 Hz, 1H), 2.59-2.29 |
| | (m, 3H), 2.11 (d, J = 9.4 Hz, 1H). (3 exchangeable protons are not seen) |
| 435 | MS m/z 404.2 [M + H]⁺; ¹H NMR (400 MHz, METHANOL-d₄) δ: 8.43- |
| | 8.40 (m, 1H), 8.33-8.28 (m, 1H), 7.47-7.42 (m, 1H), 5.77-5.72 (m, 2H), |
| | 4.87 (s, 2H), 3.38-3.32 (m, 1H), 3.19-3.10 (m, 1H), 2.54-2.45 (m, 2H), |
| | 2.39-2.30 (m, 4H), 2.14-2.01 (m, 1H). (3 exchangeable protons are |
| | not seen) |
| 436 | MS m/z 386.0 [M + H]⁺; ¹H NMR (400 MHz, METHANOL-d₄) δ: 8.57- |
| | 8.36 (m, 2H), 7.47-7.33 (m, 2H), 5.83-5.67 (m, 2H), 4.83-4.77 (m, |
| | 2H), 3.52-3.35 (m, 1H), 3.28-3.21 (m, 1H), 2.61-2.47 (m, 2H), 2.45- |
| | 2.30 (m, 4H), 2.22-2.07 (m, 1H). (3 exchangeable protons are not |
| | seen) |
| 437 | MS m/z 395.0 [M + H]⁺; ¹H NMR (400 MHz, METHANOL-d₄) δ: 7.40- |
| | 7.30 (m, 1H), 6.30-6.20 (m, 2H), 5.60-5.58 (m, 2H), 4.63 (s, 2H), 3.46 |
| | (dd, J = 9.7, 5.6 Hz, 1H), 3.30 (d, J = 4.7 Hz, 1H), 2.51-2.30 (m, 3H), |
| | 2.19-1.98 (m, 1H). (3 exchangeable protons are not seen) |
| 438 | MS m/z 440.1 [M + H]⁺; ¹H NMR (METHANOL-d₄) δ: 6.63 (s, 1H), |
| | 4.15-3.99 (m, 2H), 3.58-3.42 (m, 1H), 3.26-3.17 (m, 1H), 2.42- |
| | 2.00 (m, 4H), 1.79 (t, J = 2.3 Hz, 3H), 1.73-1.64 (m, 6H). (3, |
| | exchangeable protons not observed). |
| 439 | MS m/z 424.1 [M + H]⁺; ¹H NMR (400 MHz, METHANOL-d₄) δ: 8.55- |
| | 8.35 (m, 1H), 8.38-8.35 (m, 1H), 7.44-7.37 (m, 1H), 5.8-5.5(m, 2H), |
| | 4.76 (s, 2H), 3.11-3.02 (m, 2H), 2.2.45-2.19 (m, 2H), 2.27-2.19 (m, |
| | 1H), 2.04-1.94 (m, 1H). (3 exchangeable protons are not seen) |
| 440 | MS m/z 406.1 [M + H]⁺; ¹H NMR (400 MHz, METHANOL-d₄) δ: 8.50- |
| | 8.45(m, 2H), 7.45-7.39 (m, 2H), 5.85-5.70 (m, 2H), 4 . . . 84-4.78 (m, 2H), |
| | 3.54-3.44 (m, 1H), 3.28-3.15 (m, 1H), 2.59-2.35 (m, 3H), 2.16-2.02 |
| | (m, 1H). (3 exchangeable protons are not seen) |
| 441 | MS m/z 392.0 [M + H]⁺; ¹H NMR (400 MHz, METHANOL-d₄) δ: 7.72 |
| | (d, J = 3.4 Hz, 1H), 7.50 (d, J = 3.4 Hz, 1H), 5.87-5.73 (m, 2H), 5.1-5.02 |
| | (m, 2H), 3.57-3.49 (m, 1H), 3.36-3.47 (m, 1H), 2.64-2.52 (m, 2H), 2.50- |
| | 2.43 (m, 1H), 2.37 (s, 3H), 2.28-2.16 (m, 1H). (3 exchangeable protons |
| | are not seen) |
| 444 | MS m/z 422.4 [M + H]⁺; ¹H NMR (METHANOL-d₄) δ: 7.46-7.43 (m, |
| | 1H), 6.65-6.60 (m, 1H), 6.38-6.28 (m, 2H), 4.53 (s, 2H), 3.51-3.38 |
| | (m, 1H), 3.23-3.15 (m, 1H), 2.40-2.02 (m, 5H), 1.69 (s, 7H). (3, |
| | exchangeable protons not observed). |
| 448 | MS m/z 450.0 [M + H]⁺; ¹H NMR (METHANOL-d₄) δ: 6.54 (s, 1H), |
| | 5.79-5.68 (m, 1H), 5.55-5.46 (m, 1H), 4.10 (dd, J = 25.6, 11.5 Hz, |
| | 1H), 4.03 (s, 2H), 3.75-3.62 (m, 1H), 2.40-2.30 (m, 1H), 2.29-2.22 |
| | (m, 1H), 2.15-1.98 (m, 2H), 1.85-1.77 (m, 4H), 1.77-1.69 (m, 1H). |
| | (3 Exchangeable protons not observed). |
| 449 | MS m/z 381.0 [M + H]⁺; ¹H NMR (METHANOL-d₄) δ: 7.32 (s, 1H), |
| | 6.24 (dd, J = 8.8, 2.7 Hz, 2H), 4.65 (d, J = 9.1 Hz, 2H), 3.39 (d, J = 10.2 |
| | Hz, 1H), 3.32 (d, J = 10.5 Hz, 1H), 2.26 (s, 3H). (3, exchangeable |
| | protons not observed). |
| 450 | MS m/z 422.1 [M + H]⁺; ¹H NMR (METHANOL-d₄) δ: 7.48 (s, 1H), |
| | 6.74-6.60 (m, 1H), 6.38 (dd, J = 8.2, 2.5 Hz, 2H), 5.87-5.72 (m, 1H), |
| | 5.55 (d, J = 10.0 Hz, 1H), 4.60-4.55 (m, 2H), 3.89-3.66 (m, 1H), 3.15 |
| | (s, 1H), 2.71 (d, J = 5.9 Hz, 1H), 2.29 (s, 1H), 1.26 (t, J = 7.8 Hz, 3H), |
| | 0.97 (d, J = 7.2 Hz, 3H) (3, exchangeable protons not observed). |

### Example 14

### Preparation of Compound 24

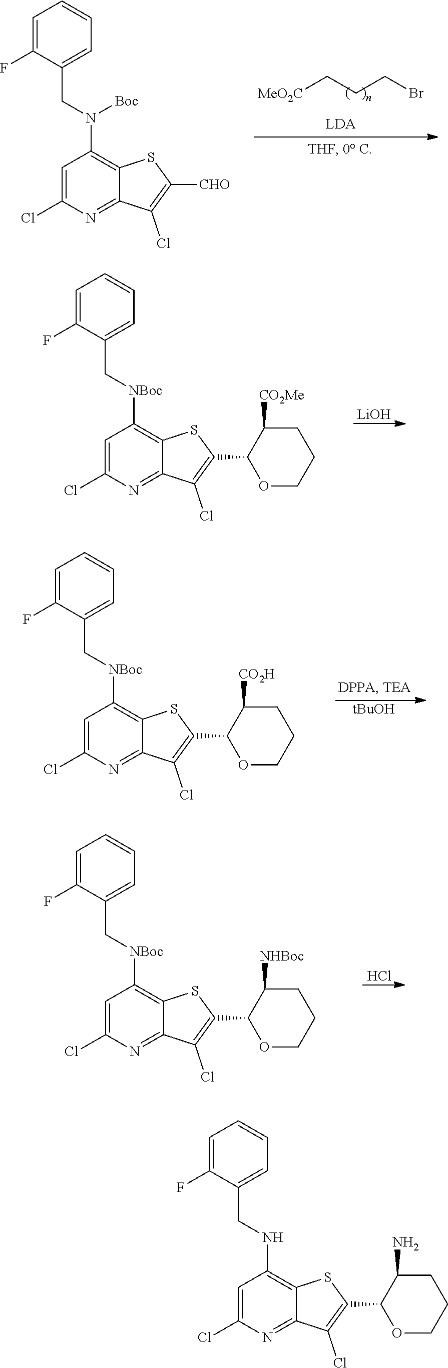

### Step 1: Methyl (2S,3S)-2-(7-((tert-butoxycarbonyl)(2-fluorobenzyl)amino)-3,5-dichlorothieno[3,2-b]pyridin-2-yl)tetrahydro-2H-pyran-3-carboxylate

To a solution of tert-butyl N-(3,5-dichloro-2-formyl-thieno[3,2-b]pyridin-7-yl)-N-[(2-fluorophenyl)methyl]carbamate (500 mg, 1.1 mmol), prepared according to the procedure for Intermediate 1, and methyl 5-bromovalerate (0.2 mL, 1 mmol) in THF (5 mL), cooled to 0° C. was added lithium diisopropylamide (2.0 M) in THF/heptane/ethylbenzene (1.2 mL, 2.4 mmol, 2.3 eq). dropwise. The ice bath was removed and the reaction was stirred at rt for 16 h. The reaction was quenched with NH₄Cl (sat. aq.). The reaction mixture was diluted with EtOAc and washed with water and brine. The combined organic layers were dried over MgSO₄, filtered, and concentrated in vacuo. The crude residue was purified by flash column chromatography on silica gel eluting with ethyl acetate in hexanes (0-40% gradient) to afford methyl (2S,3S)-2-[7-[tert-butoxycarbonyl-[(2-fluorophenyl)methyl]amino]-3,5-dichloro-thieno[3,2-b]pyridin-2-yl]tetrahydropyran-3-carboxylate (167 mg, 27% Yield) as a light yellow oil. MS m/z 568.8, 570.7 [M+H]⁺; ¹H NMR (CHLOROFORM-d) δ: 7.21-7.36 (m, 2H), 6.94-7.16 (m, 3H), 5.16 (d, J=10.1 Hz, 1H), 4.93-5.08 (m, 2H), 4.15-4.22 (m, 1H), 3.63-3.77 (m, 1H), 3.54 (s, 3H), 2.77 (ddd, J=12.0, 10.1, 3.8 Hz, 1H), 2.15-2.29 (m, 1H), 1.96-2.07 (m, 1H), 1.79 (br s, 2H), 1.43 (s, 9H).

### Step 2: (2S,3S)-2-(7-((tert-Butoxycarbonyl)(2-fluorobenzyl)amino)-3,5-dichlorothieno[3,2-b]pyridin-2-yl)tetrahydro-2H-pyran-3-carboxylic acid

A mixture of methyl (2S,3S)-2-[7-[tert-butoxycarbonyl-[(2-fluorophenyl)methyl]amino]-3,5-dichloro-thieno[3,2-b]pyridin-2-yl]tetrahydropyran-3-carboxylate (167 mg, 0.3 mmol), aqueous lithium hydroxide (2.2 mL, 2.2 mmol, 1 mol/L) and methanol (5 mL) was stirred at 50° C. for 2 hr. Cooled to rt and concentrated. Diluted with DCM and neutralized with 1M Citric acid. Organic phase was separated and concentrated to afford crude tert-butyl N-[3,5-dichloro-2-[(2S)-tetrahydropyran-2-yl]thieno[3,2-b]pyridin-7-yl]-N-[(2-fluorophenyl)methyl]carbamate;carbon dioxide (126 mg, 77% Yield) which was used in the next step without further purification. MS m/z 554.8, 556.8 [M+H]⁺.

### Step 3: tert-Butyl (2-((2S,3S)-3-((tert-butoxycarbonyl)amino)tetrahydro-2H-pyran-2-yl)-3,5-dichlorothieno[3,2-b]pyridin-7-yl)(2-fluorobenzyl)carbamate

A mixture of tert-butyl N-[3,5-dichloro-2-[(2S)-tetrahydropyran-2-yl]thieno[3,2-b]pyridin-7-yl]-N-[(2-fluorophenyl)methyl]carbamate;carbon dioxide (126 mg, 0.2 mmol), triethylamine (0.06 mL, 0.4 mmol, 2.0 eq), diphenylphosphoryl azide (0.1 mL, 0.5 mmol, 2.0 eq), and tert-butyl alcohol (2 mL) was heated at 50° C. for 1 h. The reaction mixture was then cooled to rt and concentrated. The crude residue was purified on silica gel eluting with ethyl acetates in hexane (0-40%) to afford tert-butyl N-[2-[(2S,3S)-3-(tert-butoxycarbonylamino)tetrahydropyran-2-yl]-3,5-dichloro-thieno[3,2-b]pyridin-7-yl]-N-[(2-fluorophenyl)methyl]carbamate (85 mg, 60% Yield). MS m/z 625.8, 627.6 [M+H]⁺; ¹H NMR (CHLOROFORM-d) δ: 7.22-7.27 (m, 2H), 6.96-7.12 (m, 3H), 5.02 (s, 2H), 4.75 (d, J=9.6 Hz, 1H), 4.49-4.61 (m, 1H), 4.16-4.19 (m, 1H), 3.66-3.79 (m, 1H), 3.50-3.64 (m, 1H), 2.22-2.33 (m, 1H), 1.78-1.99 (m, 2H), 1.61-1.69 (m, 1H), 1.44 (s, 9H), 1.18 (s, 9H).

### Step 4: 2-((2S,3S)-3-Aminotetrahydro-2H-pyran-2-yl)-3,5-dichloro-N-(2-fluorobenzyl)thieno[3,2-b]pyridin-7-amine

A mixture of tert-butyl N-[3,5-dichloro-2-[rac-(2S,3S)-3-(tert-butoxycarbonylamino)tetrahydropyran-2-yl]thieno[3,2-b]pyridin-7-yl]-N-[(2-fluorophenyl)methyl]carbamate (85 mg, 0.1 mmol) and HCl (4 M in dioxane) (2 mL) was stirred at rt for 2 h. Reaction mixture was concentrated and the solid was triturated with Et₂O and filtered to afford 3,5-dichloro-N-[(2-fluorophenyl)methyl]-2-[rac-(2S,3S)-3-aminotetrahydropyran-2-yl]thieno[3,2-b]pyridin-7-amine hydrochloride (72 mg, 69% Yield). MS m/z 425.9 [M+H]⁺; ¹H NMR (METHANOL-d₄) δ: 7.33-7.50 (m, 2H), 7.11-7.23 (m, 2H), 6.78 (s, 1H), 5.01 (d, J=9.8 Hz, 1H), 4.74 (s, 2H), 4.09-4.23 (m, 1H), 3.67-3.77 (m, 1H), 3.41 (td, J=10.4, 4.1 Hz, 1H), 2.28-2.41 (m, 1H), 1.82-1.97 (m, 3H) (4 exchangeable protons not observed).

The compounds below were prepared according to the procedure of Example 14 by substituting the appropriate starting materials, reagents, and reaction conditions, and followed by chiral SFC resolution if needed.

| Compound | Spectral Data |
|---|---|
| 25 | MS m/z 378.0 [M + H]⁺; ¹HNMR(400 MHz, METHANOL-d₄) δ: 7.44 |
| | (d, J = 1.1 Hz, 1H), 6.57 (s, 1H), 6.38-6.33 (m, 1H), 6.30 (d, J = 3.1 |
| | Hz, 1H), 4.53 (t, J = 4.6 Hz, 3H), 4.02 (s, 1H), 3.60 (d, J = 2.8 Hz, 1H), |
| | 2.88 (d, J = 2.0 Hz, 1H), 2.41 (s, 3H), 2.15 (d, J = 11.3 Hz, 1H), 1.85 (d, |
| | J = 50.0 Hz, 2H), 1.55 (dd, J = 11.6, 4.5 Hz, 1H). (3 exchangeable |
| | protons are not seen) |

### Example 15

### Preparation of Compound 290

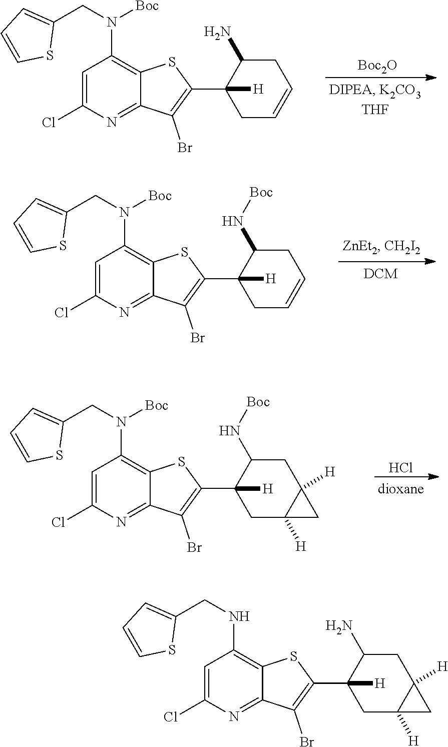

### Step 1: tert-Butyl (3-bromo-2-((1S/R,6S/R)-6-((tert-butoxycarbonyl)amino)cyclohex-3-en-1-yl)-5-chlorothieno[3,2-b]pyridin-7-yl)(thiophen-2-ylmethyl)carbamate

To a solution of tert-butyl (2-((1S/R,6S/R)-6-aminocyclohex-3-en-1-yl)-3-bromo-5-chlorothieno[3,2-b]pyridin-7-yl)(thiophen-2-ylmethyl)carbamate (201 mg, 0.36 mmol) (prepared according to Example 13 using Intermediate 20) in THF (1.8 mL) was added potassium carbonate (100 mg, 0.73 mmol, 2.0 eq) and diisopropylethylamine (0.127 mL, 0.73 mmol, 2.0 eq). The mixture was stirred until the starting material was consumed as determined by UPLCMS. The reaction mixture was separated between NH₄Cl (sat. aq.) (15 mL) and ethyl acetate (15 mL). The aqueous layer was extracted with EtOAc (15 ml×2). The combined organic layers were washed with brine, dried over MgSO₄, filtered, and then concentrated onto Celite. The crude residue was purified by flash column chromatography on silica gel eluting with ethyl acetates in hexanes to give tert-butyl (3-bromo-2-((1S/R,6S/R)-6-((tert-butoxycarbonyl)amino)cyclohex-3-en-1-yl)-5-chlorothieno[3,2-b]pyridin-7-yl)(thiophen-2-ylmethyl)carbamate (200 mg, 0.31 mmol, 84% yield) as a white solid. MS m/z 677.9 [M+Na]⁺

### Step 2: tert-Butyl (3-bromo-2-((1S/R,3S/R,4S/R,6R/S)-4-((tert-butoxycarbonyl)amino)bicyclo[4.1.0]heptan-3-yl)-5-chlorothieno[3,2-b]pyridin-7-yl)(thiophen-2-ylmethyl)carbamate

A solution of diethyl zinc (1 M in CH₂Cl, 0.46 mL, 0.46 mmol) was added to CH₂Cl₂ (0.76 mL) at 0° C. then diiodomethane (0.055 mL, 0.69 mmol) was added dropwise. After stirring for 1 h 0° C., a solution of tert-butyl (3-bromo-2-((1S/R,6S/R)-6-((tert-butoxycarbonyl)amino)cyclohex-3-en-1-yl)-5-chlorothieno[3,2-b]pyridin-7-yl)(thiophen-2-ylmethyl)carbamate (50 mg, 0.076 mmol) in CH₂Cl₂ (0.2 mL) was added and the reaction was allowed to come to 25° C. overnight with stirring. The resulting mixture was diluted in CH₂Cl₂ washed with NH₄Cl (sat. aq.) and brine, dried over MgSO₄, filtered, and then concentrated onto Celite. The crude residue was purified by flash column chromatography on silica gel eluting with ethyl acetates in hexanes to give tert-butyl (3-bromo-2-((1S/R,3S/R,4S/R,6R/S)-4-((tert-butoxycarbonyl)amino)bicyclo[4.1.0]heptan-3-yl)-5-chlorothieno[3,2-b]pyridin-7-yl)(thiophen-2-ylmethyl)carbamate (31 mg, 0.0.46 mmol, 61% yield) as a single diastereomer and a white solid. MS m/z 691.9 [M+Na]⁺

### Step 3: 2-((1S/R,3S/R,4S/R,6R/S)-4-Aminobicyclo[4.1.0]heptan-3-yl)-3-bromo-5-chloro-N-(thiophen-2-ylmethyl)thieno[3,2-b]pyridin-7-amine

A solution of tert-butyl (3-bromo-2-((1S/R,3S/R,4S/R,6R/S)-4-((tert-butoxycarbonyl)amino)bicyclo[4.1.0]heptan-3-yl)-5-chlorothieno[3,2-b]pyridin-7-yl)(thiophen-2-ylmethyl)carbamate (31 mg, 0.046 mmol) in 4 M HCl in dioxanes (1 mL) was stirred at 30° C. for 30 m. The mixture was basified with 1 M NaOH, separated between ethyl acetate and water and the free based reaction mixture was concentrated and the crude oil was purified by reverse phased C18 chromatography in TFA modified water and acetonitrile to give 2-((1 S/R,3S/R,4S/R,6R/S)-4-aminobicyclo[4.1.0]heptan-3-yl)-3-bromo-5-chloro-N-(thiophen-2-ylmethyl)thieno[3,2-b]pyridin-7-amine (15 mg, 69% yield) as an off-white solid. MS m/z [M+H]⁺470.0; ¹H NMR (METHANOL-d₄) δ: 7.33 (d, J=5.1 Hz, 1H), 7.11 (d, J=3.4 Hz, 1H), 7.00 (dd, J=5.1, 3.6 Hz, 1H), 6.66 (s, 1H), 4.79 (s, 2H), 3.50 (td, J=11.4, 6.1 Hz, 1H), 3.39-3.34 (m, 1H), 2.73-2.62 (m, 1H), 2.42-2.35 (m, 2H), 1.70 (t, J=12.3 Hz, 1H), 1.33-1.13 (m, 2H), 0.88 (td, J=8.9, 5.0 Hz, 1H), 0.36 (q, J=5.3 Hz, 1H). Trifluoroacetic acid salt, (4 exchangeable protons not observed).

### Example 16

### Preparation of Compound 342

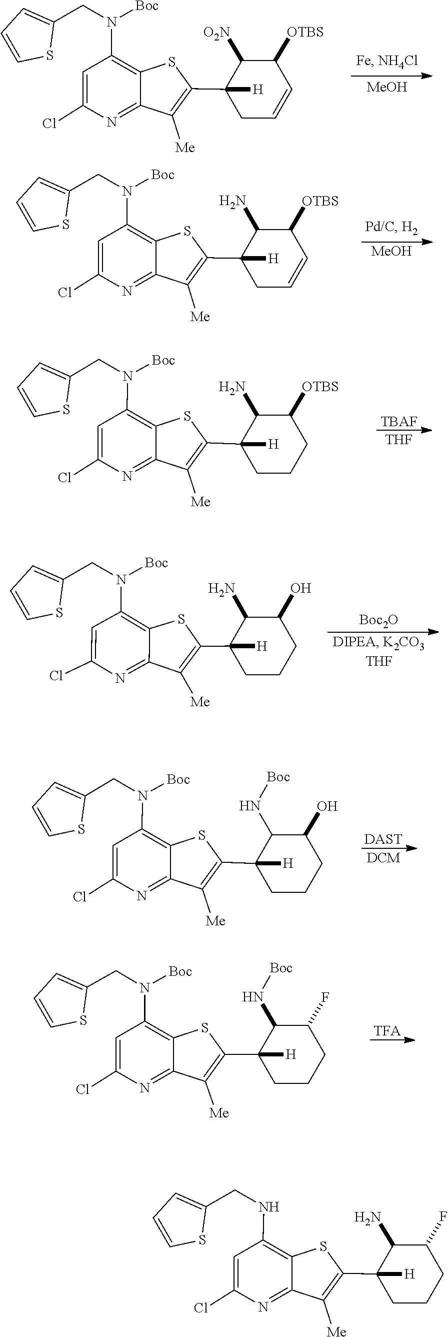

### Step 1: tert-Butyl (2-((1S/R,5S/R,6R/S)-6-amino-5-((tert-butyldimethylsilyl)oxy)cyclohex-3-en-1-yl)-5-chloro-3-methylthieno[3,2-b]pyridin-7-yl)(thiophen-2-ylmethyl)carbamate

To tert-butyl (2-((1S/R,5S/R,6R/S)-5-((tert-butyldimethylsilyl)oxy)-6-nitrocyclohex-3-en-1-yl)-5-chloro-3-methylthieno[3,2-b]pyridin-7-yl)(thiophen-2-ylmethyl)carbamate (3 g, 4.6 mmol, prepared according to Example 13), iron powder (2.06 g, 36.8 mmol, 8.0 eq), and ammonium chloride (12.3 g, 230 mmol, 50 eq) was added MeOH (46 mL) and the slurry was stirred at 55° C. until the starting material was consumed as determined by UPLCMS. The reaction mixture was filtered through Celite, concentrated in vacuo, and separated between 1 M NaOH (200 mL) and ethyl acetate (200 mL). The aqueous layer was extracted with EtOAc (100 ml×2). The combined organic layers were washed with brine, dried over MgSO₄, filtered, and then concentrated to give tert-butyl (2-((1S/R,5S/R,6R/S)-6-amino-5-((tert-butyldimethylsilyl)oxy)cyclohex-3-en-1-yl)-5-chloro-3-methylthieno[3,2-b]pyridin-7-yl)(thiophen-2-ylmethyl)carbamate crudely, as an orange semisolid (2.6 g) which was used without further purification. MS m/z 620.6 [M+H]⁺.

### Step 2: tert-Butyl (2-((1S/R,2R/S,3S/R)-2-amino-3-((tert-butyldimethylsilyl)oxy)cyclohexyl)-5-chloro-3-methylthieno[3,2-b]pyridin-7-yl)(thiophen-2-ylmethyl)carbamate

Crude tert-butyl (2-((1S/R,5S/R,6R/S)-6-amino-5-((tert-butyldimethylsilyl)oxy)cyclohex-3-en-1-yl)-5-chloro-3-methylthieno[3,2-b]pyridin-7-yl)(thiophen-2-ylmethyl)carbamate (750 mg, 1.2 mmol) was added as a solution in MeOH (2 mL) to a slurry of 20% w/w palladium on carbon (300 mg) in MeOH (5 mL) under an inert atmosphere. The resulting mixture was vacuum sparged with nitrogen and the hydrogen before being pressurized to 7 atm under an atmosphere of hydrogen and left under agitation for 5 d. The reaction mixture was filtered through Celite, and concentrated in vacuo to give crude tert-butyl (2-((1S/R,2R/S,3S/R)-2-amino-3-((tert-butyldimethylsilyl)oxy)cyclohexyl)-5-chloro-3-methylthieno[3,2-b]pyridin-7-yl)(thiophen-2-ylmethyl)carbamate as a colorless semisolid (746 mg) which was used without further purification. MS m/z 622.5 [M+H]⁺.

### Step 3: tert-Butyl (2-((1S/R,5S/R,6R/S)-6-amino-5-hydroxycyclohexyl)-5-chloro-3-methylthieno[3,2-b]pyridin-7-yl)(thiophen-2-ylmethyl)carbamate

To crude tert-butyl (2-((1S/R,2R/S,3S/R)-2-amino-3-((tert-butyldimethylsilyl)oxy)cyclohexyl)-5-chloro-3-methylthieno[3,2-b]pyridin-7-yl)(thiophen-2-ylmethyl)carbamate (480 mg, 0.77 mmol) in THF (7.7 mL) at 0° C. was added a solution of TBAF (1 M in THF, 2.3 mL, 3 eq) dropwise over 15 min. The reaction was quenched with saturated aqueous sodium bicarbonate and extracted with EtOAc (3×15 mL). The combined organic layers were washed with brine, dried over MgSO₄, and concentrated in vacuo onto Celite before being purified by silica gel chromatography in a DCM/MeOH gradient to provide tert-butyl (2-((1S/R,2R/S,3S/R)-2-amino-3-hydroxycyclohexyl)-5-chloro-3-methylthieno[3,2-b]pyridin-7-yl)(thiophen-2-ylmethyl)carbamate as a colorless semisolid (320 mg). MS m/z 622.5 [M+H]⁺; ¹H NMR (CHLOROFORM-d) δ: 7.15 (d, J=5.1 Hz, 1H), 6.91 (s, 1H), 6.79 (t, J=4.4 Hz, 1H), 6.70 (d, J=3.4 Hz, 1H), 5.01-4.85 (m, 2H), 3.36 (s, 1H), 3.02-2.91 (m, 1H), 2.58 (s, 1H), 2.34 (s, 3H), 2.00 (d, J=11.4 Hz, 1H), 1.89-1.71 (m, 2H), 1.70-1.62 (m, 2H), 1.38 (s, 10H). (3 exchangeable protons not observed).

### Step 4: tert-Butyl (2-((1S/R,2R/S,3S/R)-2-((tert-butoxycarbonyl)amino)-3-hydroxycyclohexyl)-5-chloro-3-methylthieno[3,2-b]pyridin-7-yl)(thiophen-2-ylmethyl)carbamate

To tert-butyl (2-((1S/R,2R/S,3S/R)-2-amino-3-hydroxycyclohexyl)-5-chloro-3-methylthieno[3,2-b]pyridin-7-yl)(thiophen-2-ylmethyl)carbamate (160 mg, 0.31 mmol), DMAP (19 mg, 0.16 mmol, 0.5 eq), and potassium carbonate (131 mg, 0.95 mmol, 3 eq) in THF (3.1 mL) at 0° C. was added a solution of Boc anhydride (1 M in DCM, 0.47 mL, 1.5 eq). The reaction allowed to come to room temperature with stirring until the substrate was found to be consumed by UPLCMS at which point the reaction was separated between saturated aqueous sodium bicarbonate and EtOAc. The aqueous phase was extracted with EtOAc (3×15 mL). The combined organic layers were washed with brine, dried over MgSO₄, and concentrated in vacuo onto Celite before being purified by silica gel chromatography in an EtOAc/hexanes gradient to provide tert-butyl (2-((1S/R,2R/S,3S/R)-2-((tert-butoxycarbonyl)amino)-3-hydroxycyclohexyl)-5-chloro-3-methylthieno[3,2-b]pyridin-7-yl)(thiophen-2-ylmethyl)carbamate as a colorless semisolid (100 mg, 52% yield). MS m/z 608.1 [M+H]⁺; ¹H NMR (CHLOROFORM-d) δ: 7.24 (d, J=5.0 Hz, 1H), 6.98 (s, 1H), 6.89 (t, J=4.2 Hz, 1H), 6.80 (s, 1H), 5.05 (s, 2H), 4.43 (s, 1H), 3.74 (s, 1H), 3.43 (t, J=9.2 Hz, 1H), 3.30 (s, 1H), 2.45 (s, 3H), 2.01 (d, J=13.2 Hz, 1H), 1.91 (s, 1H), 1.66-1.45 (m, 13H), 1.27 (s, 9H).

### Step 5: tert-Butyl (2-((1S/R,2R/S,3R/S)-2-((tert-butoxycarbonyl)amino)-3-fluorocyclohexyl)-5-chloro-3-methylthieno[3,2-b]pyridin-7-yl)(thiophen-2-ylmethyl)carbamate

To tert-butyl (2-((1S/R,2R/S,3 S/R)-2-amino-3-hydroxycyclohexyl)-5-chloro-3-methylthieno[3,2-b]pyridin-7-yl)(thiophen-2-ylmethyl)carbamate (24.5 mg, 0.04 mmol) in DCM (0.4 mL) at 0° C. in a PTFE vial was added Diethylaminosulfur trifluoride (DAST, 1 M in DCM, 0.052 mL, 0.052 mmol, 1.3 eq) and the reaction was held at 0° C. with stirring for 7 h at which point the reaction was quenched with aqueous sodium bicarbonate and extracted with DCM (2×10 mL). The combined organic layers were washed with aqueous sodium bicarbonate, and brine before being dried over MgSO₄, and concentrated in vacuo to yield a crude mixture containing tert-butyl (2-((1S/R,2R/S,3R/S)-2-((tert-butoxycarbonyl)amino)-3-fluorocyclohexyl)-5-chloro-3-methylthieno[3,2-b]pyridin-7-yl)(thiophen-2-ylmethyl)carbamate (MS m/z 610.0 [M+H]⁺) which was used without further purification.

### Step 6: 2-((1S/R,2R/S,3R/S)-2-Amino-3-fluorocyclohexyl)-5-chloro-3-methyl-N-(thiophen-2-ylmethyl)thieno[3,2-b]pyridin-7-amine

To crude tert-butyl (2-((1S/R,2R/S,3R/S)-2-((tert-butoxycarbonyl)amino)-3-fluorocyclohexyl)-5-chloro-3-methylthieno[3,2-b]pyridin-7-yl)(thiophen-2-ylmethyl)carbamate was added TFA (0.5 mL) and the solution was heated to 40° C. with stirring for 15 min. The reaction was purified by a C18 reverse phased chromatography in TFA modified water/MeCN gradient to yield 2-((1S/R,2R/S,3R/S)-2-amino-3-fluorocyclohexyl)-5-chloro-3-methyl-N-(thiophen-2-ylmethyl)thieno[3,2-b]pyridin-7-amine (5 mg) MS m/z [M+H]409.9; ¹H NMR (METHANOL-d₄) δ: 7.20 (dd, J=5.1, 1.2 Hz, 1H), 6.98 (dd, J=3.5, 1.3 Hz, 1H), 6.88 (dd, J=5.1, 3.5 Hz, 1H), 6.49 (s, 1H), 5.10-4.88 (m, 1H), 4.67 (s, 2H), 3.65-3.44 (m, 2H), 2.30 (s, 3H), 2.19 (s, 1H), 2.00 (d, J=11.4 Hz, 1H), 1.86-1.61 (m, 4H). Trifluoroacetic acid salt, (3 exchangeable protons not observed).

The compounds below were prepared according to the procedure of Example 16 by substituting the appropriate starting materials, reagents, and reaction conditions, and followed by chiral SFC resolution if needed.

| Compound | Spectral Data |
|---|---|
| 181 | MS m/z [M − H]⁻ 473.8; ¹H NMR (METHANOL-d₄) δ: 7.26-7.15 (m, |
| | 1H), 6.99 (d, J = 3.4 Hz, 1H), 6.88 (dd, J = 5.1, 3.5 Hz, 1H), 6.49 (s, |
| | 1H), 4.70-4.46 (m, 3H), 3.28 (s, 1H), 2.90 (s, 1H), 2.14 (dt, J = 9.3, |
| | 4.4 Hz, 1H), 1.95-1.77 (m, 2H), 1.54 (t, J = 11.9 Hz, 1H), 1.43 (q, J = |
| | 14.1 Hz, 1H), 1.35-1.22 (m, 1H). (3, exchangeable protons not |
| | observed). |
| 25 | MS m/z 378.0 [M + H]+; 1HNMR(400 MHz, METHANOL-d4) δ: 7.44 |
| | (d, J = 1.1 Hz, 1H), 6.57 (s, 1H), 6.38-6.33 (m, 1H), 6.30 (d, J = 3.1 |
| | Hz, 1H), 4.53 (t, J = 4.6 Hz, 3H), 4.02 (s, 1H), 3.60 (d, J = 2.8 Hz, 1H), |
| | 2.88 (d, J = 2.0 Hz, 1H), 2.41 (s, 3H), 2.15 (d, J = 11.3 Hz, 1H), 1.85 (d, |
| | J = 50.0 Hz, 2H), 1.55 (dd, J = 11.6, 4.5 Hz, 1H). (3 exchangeable |
| | protons are not seen) |
| 335 | MS m/z [M − H]⁻ 473.8; ¹H NMR (METHANOL-d₄) δ: 7.32 (d, J = 5.1 |
| | Hz, 1H), 7.11 (d, J = 3.4 Hz, 1H), 7.00 (dd, J = 5.1, 3.5 Hz, 1H), 6.60 (s, |
| | 1H), 5.25-5.01 (m, 1H), 4.78 (s, 2H), 3.82-3.67 (m, 1H), 3.60 (s, |
| | 1H), 2.33-2.22 (m, 1H), 1.95-1.64 (m, 5H). (3, exchangeable protons |
| | not observed). |

### Example 17

### Preparation of Compound 371

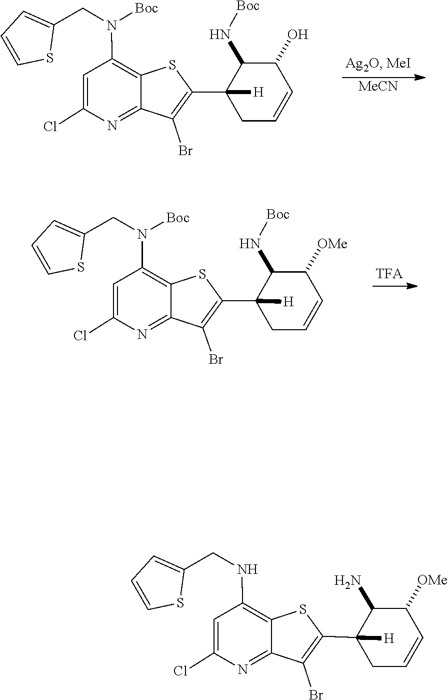

### Step 1: tert-Butyl (3-bromo-2-((1S/R,5R/S,6R/S)-6-((tert-butoxycarbonyl)amino)-5-methoxycyclohex-3-en-1-yl)-5-chlorothieno[3,2-b]pyridin-7-yl)(thiophen-2-ylmethyl)carbamate

To tert-butyl (3-bromo-2-((1S/R,5R/S,6R/S)-6-((tert-butoxycarbonyl)amino)-5-hydroxycyclohex-3-en-1-yl)-5-chlorothieno[3,2-b]pyridin-7-yl)(thiophen-2-ylmethyl)carbamate (46.8 mg, 0.070 mmol, prepared according to Example 13), and silver (I) oxide (80.8 mg, 0.35 mmol, 5.0 eq) in acetonitrile (0.35 mL) was added methyl iodide (0.043 mL, 0.70 mmol, 10 eq) and the resulting mixture proceeded with stirring for 3 d. The crude slurry was separated between saturated aqueous sodium bicarbonate and ethyl acetate, extracted with ethyl acetate and the combined organic layers were washed with brine, dried over MgSO₄, and concentrated onto Celite. Purification by silica gel chromatography in gradient EtOAc/hexanes yielded tert-butyl (3-bromo-2-((1S/R,5R/S,6R/S)-6-((tert-butoxycarbonyl)amino)-5-methoxycyclohex-3-en-1-yl)-5-chlorothieno[3,2-b]pyridin-7-yl)(thiophen-2-ylmethyl)carbamate (23 mg, 48% yield) as a colorless semisolid. MS m/z [M+H]⁺ 685.8; ¹H NMR (CHLOROFORM-d) δ: 7.25 (dd, J=5.1, 1.3 Hz, 1H), 7.03 (s, 1H), 6.90 (dd, J=5.1, 3.5 Hz, 1H), 6.82-6.74 (m, 1H), 5.93-5.80 (m, 2H), 5.13-4.90 (m, 2H), 4.58 (s, 1H), 4.16 (dd, J=10.6, 6.5 Hz, 1H), 3.94 (q, J=7.4 Hz, 2H), 3.44 (s, 3H), 2.62-2.44 (m, 2H), 1.48 (s, 9H), 1.21 (s, 9H).

### Step 2: 2-((1S/R,5R/S,6R/S)-6-Amino-5-methoxycyclohex-3-en-1-yl)-3-bromo-5-chloro-N-(thiophen-2-ylmethyl)thieno[3,2-b]pyridin-7-amine

To tert-butyl (3-bromo-2-((1S/R,5R/S,6R/S)-6-((tert-butoxycarbonyl)amino)-5-methoxycyclohex-3-en-1-yl)-5-chlorothieno[3,2-b]pyridin-7-yl)(thiophen-2-ylmethyl)carbamate (23 mg, 0.057 mmol) was added 4 M HCl in dioxane (0.5 mL) and the solution was heated to 40° C. with stirring for 30 min before 2 mL of diethyl ether was added. The resulting precipitate was filtered and washed with ether to yield (2R/S,3S/R)-2-amino-3-(3-bromo-5-chloro-7-((thiophen-2-ylmethyl)amino)thieno[3,2-b]pyridin-2-yl)cyclohexan-1-one (31 mg, 90% yield) MS m/z [M+H]⁺471.7; ¹H NMR (DMSO-d₆) δ: 8.30-8.20 (m, 2H), 8.16 (t, J=6.0 Hz, 1H), 7.42 (dd, J=5.1, 1.3 Hz, 1H), 7.17-7.06 (m, 1H), 7.00 (dd, J=5.1, 3.5 Hz, 1H), 6.69 (s, 1H), 4.82-4.62 (m, 3H), 4.54 (s, 1H), 3.72 (t, J=11.7 Hz, 1H), 3.57 (s, 1H), 2.88 (td, J=14.0, 6.0 Hz, 1H), 2.28-2.06 (m, 3H), 1.76 (t, J=13.6 Hz, 1H). HCl salt.

### Example 18

### Preparation of Compound 192

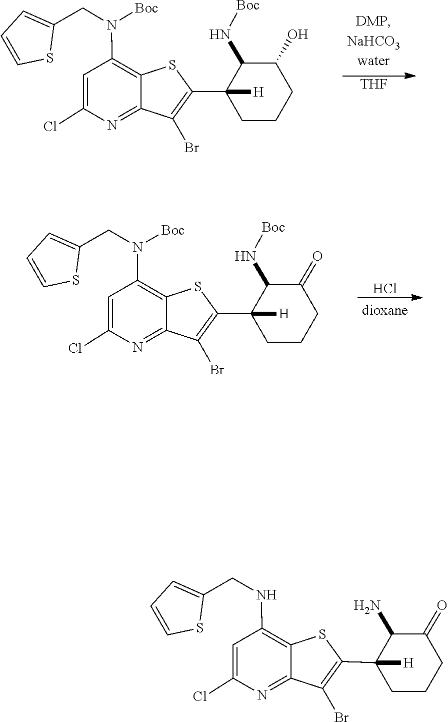

### Step 1 tert-Butyl (3-bromo-2-((1S/R,2R/S)-2-((tert-butoxycarbonyl)amino)-3-oxocyclohexyl)-5-chlorothieno[3,2-b]pyridin-7-yl)(thiophen-2-ylmethyl)carbamate

To tert-butyl (3-bromo-2-((1S/R,2R/S,3R/S)-2-((tert-butoxycarbonyl)amino)-3-hydroxycyclohexyl)-5-chlorothieno[3,2-b]pyridin-7-yl)(thiophen-2-ylmethyl)carbamate (49 mg, 0.073 mmol, prepared according to Example 13), sodium bicarbonate (61.2 mg, 0.73 mmol, 10.0 eq), and Dess-Martin periodinane (DMP, 46.3 mg, 0.11 mmol, 1.5 eq) in DCM (0.35 mL) was added water (0.0013 mL, 0.073 mmol, 1.0 eq) and the resulting mixture proceeded with stirring for 2 h. The reaction was quenched with 1:1 saturated aqueous sodium thiosulfate: saturated aqueous sodium bicarbonate (2 mL) with stirring for 30 m. The crude slurry was separated between water and DCM, extracted with DCM and the combined organic layers were washed with brine, dried over MgSO₄, and concentrated onto Celite. Purification by silica gel chromatography in gradient EtOAc/hexanes yielded tert-butyl (3-bromo-2-((1S/R,2R/S)-2-((tert-butoxycarbonyl)amino)-3-oxocyclohexyl)-5-chlorothieno[3,2-b]pyridin-7-yl)(thiophen-2-ylmethyl)carbamate (38 mg, 78% yield) as a colorless semisolid. ¹H NMR (CHLOROFORM-d) δ: 7.46-6.53 (m, 4H), 5.38-4.86 (m, 2H), 4.77-4.37 (m, 1H), 3.61 (td, J=12.1, 3.6 Hz, 1H), 2.95-2.47 (m, 2H), 2.47-2.09 (m, 3H), 2.02-1.71 (m, 1H), 1.48 (s, 9H), 1.21 (s, 9H).

### Step 2: (2R/S,3S/R)-2-Amino-3-(3-bromo-5-chloro-7-((thiophen-2-ylmethyl)amino)thieno[3,2-b]pyridin-2-yl)cyclohexan-1-one

To tert-butyl (3-bromo-2-((1S/R,2R/S)-2-((tert-butoxycarbonyl)amino)-3-oxocyclohexyl)-5-chlorothieno[3,2-b]pyridin-7-yl)(thiophen-2-ylmethyl)carbamate (38 mg, 0.035) was added TFA (0.5 mL) and the solution was heated to 40° C. with stirring for 15 min.

The reaction was purified by a C18 reverse phased chromatography in TFA modified water/MeCN gradient to yield 2-((1S/R,5R/S,6R/S)-6-amino-5-methoxycyclohex-3-en-1-yl)-3-bromo-5-chloro-N-(thiophen-2-ylmethyl)thieno[3,2-b]pyridin-7-amine (30 mg, 89% yield) MS m/z [M+H]⁺486.0; ¹H NMR (DMSO-d₆) δ: 8.13 (s, 3H), 7.41 (dd, J=5.1, 1.3 Hz, 1H), 7.12 (d, J=3.4 Hz, 1H), 7.00 (dd, J=5.1, 3.4 Hz, 1H), 6.68 (d, J=2.1 Hz, 1H), 5.95 (d, J=11.7 Hz, 2H), 4.75 (d, J=5.0 Hz, 2H), 4.19 (s, 1H), 3.73 (s, 1H), 3.57 (s, 1H), 3.40 (s, 3H) 2.60-2.52 (m, 2H).

### Example 19

### Preparation of Compound 307

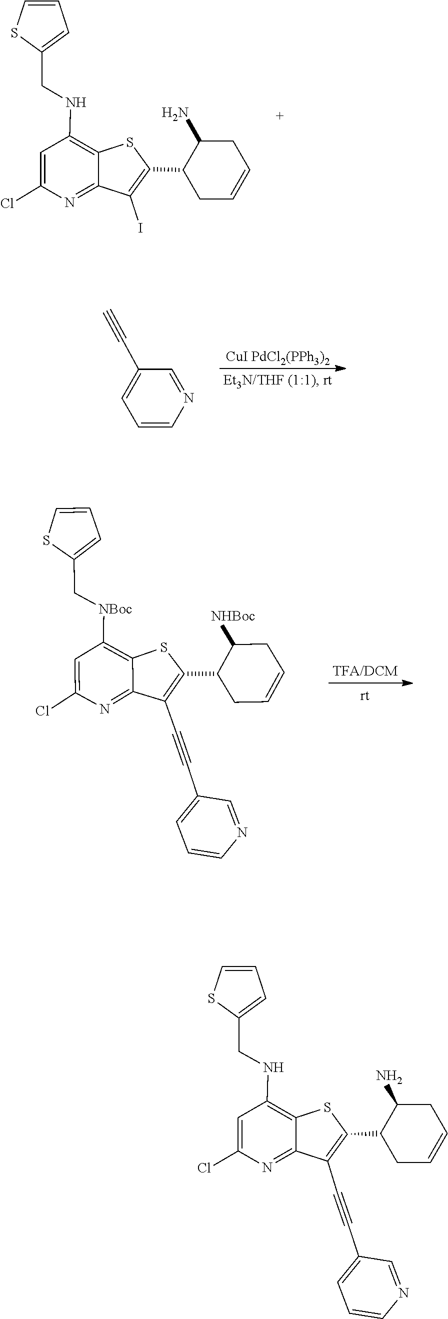

### Step 1: rac-tert-Butyl (2-((1S,6S)-6-((tert-butoxycarbonyl)amino)cyclohex-3-en-1-yl)-5-chloro-3-(pyridin-3-ylethynyl)thieno[3,2-b]pyridin-7-yl)(thiophen-2-ylmethyl)carbamate

To an oven dried 10 mL vial was added Pd(PPh₃)₂Cl₂ (4 mg, 0.006 mmol), CuI (2 mg, 0.012 mmol) and rac-2-((1S,6S)-6-aminocyclohex-3-en-1-yl)-5-chloro-3-iodo-N-(thiophen-2-ylmethyl)thieno[3,2-b]pyridin-7-amine (70 mg, 0.099 mmol). Placed this vial under Argon flow for 10 minutes. 3-ethynylpyridine (103 mg, 0.996 mmol), Et₃N (0.67 mL), and THF (0.67 mL) were then added, and the solution was left to stir overnight under argon atmosphere. The reaction mixture was diluted with ethyl acetate (5 mL) and then washed with water (2×5 mL) followed by brine solution (5 mL). Dried over anhydrous Na₂SO₄, filtered and concentrated under reduced pressure. The resulting residue was purified by silica gel column chromatography with ethyl acetate in hexanes (5 to 70% gradient) to give rac-tert-butyl (2-((1S,6S)-6-((tert-butoxycarbonyl)amino)cyclohex-3-en-1-yl)-5-chloro-3-(pyridin-3-ylethynyl)thieno[3,2-b]pyridin-7-yl)(thiophen-2-ylmethyl)carbamate as a white solid (48.4 mg, 72% yield). MS m/z 677.3 [M+H]⁺.

### Step 2: rac-2-((1S,6S)-6-Aminocyclohex-3-en-1-yl)-5-chloro-3-(pyridin-3-ylethynyl)-N-(thiophen-2-ylmethyl)thieno[3,2-b]pyridin-7-amine

To a DCM (1.5 mL) solution of rac-tert-butyl (2-((1S,6S)-6-((tert-butoxycarbonyl)amino)cyclohex-3-en-1-yl)-5-chloro-3-(pyridin-3-ylethynyl)thieno[3,2-b]pyridin-7-yl)(thiophen-2-ylmethyl)carbamate (48 mg, 0.071 mmol, 1.0 eq.) was added trifluoroacetic acid (1.5 mL) at room temperature. After stirring for 1 h, the mixture was concentrated in vacuo. The residue was purified by reverse phase column chromatography with acetonitrile (0.1% trifluoroacetic acid) in water (0.1% trifluoroacetic acid) (0 to 70% gradient) to give rac-2-((1S,6S)-6-aminocyclohex-3-en-1-yl)-5-chloro-3-(pyridin-3-ylethynyl)-N-(thiophen-2-ylmethyl)thieno[3,2-b]pyridin-7-amine. MS m/z 477.3 [M+H]⁺; ¹H NMR (400 MHz, METHANOL-d₄) δ: 8.92 (s, 1H), 8.62 (d, J=5.0 Hz, 1H), 8.36-8.22 (m, 1H), 7.64-7.61 (m, 1H), 7.32 (d, J=5.0 Hz, 1H), 7.11 (d, J=3.0 Hz, 1H), 7.00-6.98 (m, 1H), 6.67 (s, 1H), 5.94 (d, J=10.2 Hz, 1H), 5.82 (d, J=10.1 Hz, 1H), 4.79 (s, 2H), 4.00-3.94 (m, 1H), 3.91-3.74 (m, 1H), 2.83-2.72 (m, 2H), 2.71-2.63 (m, 1H), 2.45-2.28 (m, 1H). Trifluoroacetic acid salt (4 exchangeable protons not seen).

The compounds below were prepared according to the procedure of Example 19 by substituting the appropriate starting materials, reagents, and reaction conditions, and followed by chiral SFC resolution if needed.

| Compound | Spectral Data |
|---|---|
| 175 | MS m/z 458.1 [M + H]⁺; ¹H NMR (METHANOL-d₄) δ: 7.30 (dd, J = 5.0, |
| | 1.3 Hz, 1H), 7.09 (dd, J = 3.5, 1.3 Hz, 1H), 6.98 (dd, J = 5.1, 3.5 Hz, |
| | 1H), 6.61 (s, 1H), 5.91 (d, J = 10.1 Hz, 1H), 5.78 (d, J = 10.1 Hz, 1H), |
| | 4.76 (s, 2H), 3.94 (td, J = 9.5, 5.4 Hz, 1H), 3.74 (t, J = 6.0 Hz, 2H), 3.67 |
| | (td, J = 9.8, 5.9 Hz, 1H), 2.80-2.60 (m, 5H), 2.38-2.28 (m, 1H), 1.92- |
| | 1.82 (m, 2H). (4 Exchangeable protons not observed). |
| 177 | MS m/z 444.1 [M + H]⁺; ¹H NMR (METHANOL-d₄) δ: 7.31 (dd, J = 5.1, |
| | 1.2 Hz, 1H), 7.09 (dd, J = 3.5, 1.2 Hz, 1H), 6.98 (dd, J = 5.1, 3.5 Hz, |
| | 1H), 6.62 (s, 1H), 5.94-5.86 (m, 1H), 5.82-5.74 (m, 1H), 4.77 (s, |
| | 2H), 3.94-3.87 (m, 1H), 3.82 (t, J = 6.0 Hz, 2H), 3.79-3.62 (m, 1H), |
| | 2.76 (t, J = 6.0 Hz, 2H), 2.72-2.56 (m, 3H), 2.41-2.26 (m, 1H). (4 |
| | Exchangeable protons not observed). |
| 189 | MS m/z [M + H]⁺ 416.5; ¹H NMR (METHANOL-d₄) δ: 8.44 (s, 1H), 7.20 |
| | (d, J = 5.1 Hz, 1H), 6.98 (d, J = 3.4 Hz, 1H), 6.88 (t, J = 4.4 Hz, 1H), |
| | 6.47 (s, 1H), 4.65 (s, 2H), 3.29 (s, 1H), 3.16 (d, J = 10.8 Hz, 1H), 2.07 |
| | (s, 4H), 1.98 (d, J = 13.5 Hz, 1H), 1.89-1.74 (m, 2H), 1.67 (t, J = 12.2 |
| | Hz, 1H), 1.39 (q, J = 11.4 Hz, 3H). Formic acid salt, (3, exchangeable |
| | protons not observed). |
| 208 | MS m/z 452.1 [M + H]⁺; ¹H NMR (METHANOL-d₄) δ: 7.30 (dd, J = 5.0, |
| | 1.5 Hz, 1H), 7.09 (dd, J = 3.6, 1.5 Hz, 1H), 7.01-6.95 (m, 1H), 6.62 (s, |
| | 1H), 4.77 (s, 2H), 4.02 (dd, J = 25.9, 11.7 Hz, 1H), 3.60-3.55 (m, 1H), |
| | 2.36-2.29 (m, 1H), 2.25 (d, J = 12.8 Hz, 1H), 2.18 (s, 3H), 2.13-1.94 |
| | (m, 2H), 1.86-1.66 (m, 2H). (3 Exchangeable protons not observed). |
| 210 | MS m/z 396.1 [M + H]+; ¹H NMR (METHANOL-d₄) δ: 7.37 (d, J = 6.4 |
| | Hz, 4H), 7.29 (d, J = 6.9 Hz, 1H), 6.39 (d, J = 11.0 Hz, 1H), 4.59 (s, |
| | 2H), 3.94 (s, 1H), 3.19 (d, J = 12.2 Hz, 1H), 3.02 (d, J = 11.0 Hz, 1H), |
| | 2.08 (t, J = 12.5 Hz, 2H), 1.89 (s, 2H), 1.71 (d, J = 12.9 Hz, 1H), 1.53- |
| | 1.35 (m, 3H) (3, exchangeable protons not observed). |
| 211 | MS m/z 474.1 [M + H]⁺; ¹H NMR (METHANOL-d₄) δ: 7.31 (d, J = 5.1 |
| | Hz, 1H), 7.10-7.06 (m, 1H), 7.01-6.95 (m, 1H), 6.62 (s, 1H), 5.90 (d, |
| | J = 10.1 Hz, 1H), 5.79 (d, J = 10.1 Hz, 1H), 4.76 (s, 2H), 3.96-3.83 |
| | (m, 2H), 3.77-3.69 (m, 1H), 3.64 (t, J = 5.6 Hz, 2H), 2.88-2.80 (m, |
| | 1H), 2.75 (d, J = 6.4 Hz, 1H), 2.71 (d, J = 6.4 Hz, 1H), 2.68-2.56 (m, |
| | 2H), 2.40-2.30 (m, 1H). (5 Exchangeable protons not observed). |
| 213 | MS m/z 448.1 [M + H]⁺; ¹H NMR (METHANOL-d₄) δ: 7.33-7.28 (m, |
| | 1H), 7.09 (s, 1H), 7.00-6.95 (m, 1H), 6.62 (s, 1H), 5.01 (d, J = 9.8 Hz, |
| | 1H), 4.77 (s, 2H), 4.15 (d, J = 11.6 Hz, 1H), 3.90-3.80 (m, 2H), 3.67 |
| | (t, J = 11.3 Hz, 1H), 3.36 (d, J = 10.9 Hz, 1H), 2.82-2.75 (m, 2H), 2.31 |
| | (d, J = 11.7 Hz, 1H), 2.00-1.80 (m, 3H). (4 Exchangeable protons not |
| | observed). |
| 216 | MS m/z 474.1 [M + H]⁺; ¹H NMR (METHANOL-d₄) δ: 7.30 (d, J = 5.1 |
| | Hz, 1H), 7.09 (d, J = 3.5 Hz, 1H), 7.01-6.95 (m, 1H), 6.62 (s, 1H), |
| | 5.90 (d, J = 10.4 Hz, 1H), 5.78 (d, J = 10.6 Hz, 1H), 4.76 (s, 2H), 3.96- |
| | 3.83 (m, 2H), 3.77-3.69 (m, 1H), 3.64 (t, J = 5.5 Hz, 2H), 2.88-2.80 |
| | (m, 1H), 2.75 (d, J = 6.4 Hz, 1H), 2.71 (d, J = 6.4 Hz, 1H), 2.68-2.56 |
| | (m, 2H), 2.40-2.30 (m, 1H). (5 Exchangeable protons not observed). |
| 226 | MS m/z [M + H]⁺ 410.5; ¹H NMR (METHANOL-d₄) δ: 8.45 (s, 1H), 7.29- |
| | 7.20 (m, 4H), 7.17 (d, J = 6.5 Hz, 1H), 6.29 (s, 1H), 4.47 (s, 2H), 3.13 |
| | (d, J = 9.3 Hz, 2H), 2.13-1.91 (m, 5H), 1.79 (d, J = 16.3 Hz, 2H), 1.65 |
| | (d, J = 12.7 Hz, 1H), 1.39 (q, J = 16.7 Hz, 3H). Formic acid salt, (3, |
| | exchangeable protons not observed). |
| 227 | MS m/z 418.1 [M + H]⁺; ¹H NMR (METHANOL-d₄) δ: 8.54 (s, 1H, |
| | formic acid), 7.30 (dt, J = 5.0, 1.5 Hz, 1H), 7.08 (dd, J = 3.5, 1.5 Hz, |
| | 1H), 6.98 (dd, J = 5.1, 3.5 Hz, 1H), 6.55 (s, 1H), 4.75 (s, 2H), 4.15- |
| | 4.00 (m, 2H), 3.68-3.51 (m, 2H), 3.43-3.34 (m, 1H), 3.26 (dd, J = |
| | 10.4, 10.4 Hz, 1H), 3.17 (dd, J = 10.4, 4.3 Hz, 1H), 2.18 (s, 3H), 2.05- |
| | 1.98 (m, 1H). (3 Exchangeable protons not observed). |
| 235 | MS m/z 472.1 [M + H]⁺; ¹H NMR (METHANOL-d₄) δ: 7.30 (dd, J = 5.1, |
| | 1.2 Hz, 1H), 7.09 (dd, J = 3.5, 1.2 Hz, 1H), 6.98 (dd, J = 5.1, 3.5 Hz, |
| | 1H), 6.60 (s, 1H), 5.90 (d, J = 10.2 Hz, 1H), 5.79 (d, J = 10.2 Hz, 1H), |
| | 4.76 (s, 2H), 4.04-3.94 (m, 1H), 3.70-3.58 (m, 3H), 2.82-2.70 (m, |
| | 1H), 2.68-2.56 (m, 4H), 2.36-2.25 (m, 1H), 1.80-1.69 (m, 4H). (4 |
| | Exchangeable protons not observed). |
| 252 | MS m/z 434.1 [M + H]⁺; ¹H NMR (METHANOL-d₄) δ: 7.45 (d, J = 4.9 |
| | Hz, 1H), 7.23 (d, J = 3.5 Hz, 1H), 7.13 (dd, J = 4.9, 3.5 Hz, 1H), 6.74 (s, |
| | 1H), 5.13 (d, J = 9.8 Hz, 1H), 4.90 (s, 2H), 4.69 (s, 2H), 4.28 (d, J = |
| | 11.8 Hz, 1H), 3.81 (t, J = 10.5 Hz, 1H), 3.55 (t, J = 10.9 Hz, 1H), 2.48 |
| | (d, J = 11.2 Hz, 1H), 2.13-1.95 (m, 3H). (4 Exchangeable protons not |
| | observed). |
| 255 | MS m/z 419.1 [M + H]⁺; ¹H NMR (METHANOL-d₄) δ: 7.44 (s, 1H), |
| | 6.40-6.33 (m, 2H), 4.99 (d, J = 9.9 Hz, 1H), 4.82-4.68 (m, 2H), 4.53 |
| | (s, 2H), 4.14 (d, J = 11.8 Hz, 1H), 3.72-3.62 (m, 1H), 2.31 (d, J = 10.9 |
| | Hz, 1H), 1.99-1.80 (m, 3H). (3 Exchangeable protons not observed). |
| 311 | MS m/z 413.9 [M + H]⁺; ¹H NMR (METHANOL-d₄) δ: 7.41-7.31 (m, |
| | 2H), 7.16 (t, J = 7.5 Hz, 2H), 6.44 (s, 1H), 4.64 (s, 2H), 3.94 (s, 1H), |
| | 3.24-3.12 (m, 1H), 2.99 (s, 1H), 2.06 (d, J = 13.0 Hz, 2H), 1.89 (s, |
| | 2H), 1.70 (d, J = 12.6 Hz, 1H), 1.43 (dd, J = 45.3, 12.1 Hz, 3H) (3, |
| | exchangeable protons not observed). |
| 317 | MS m/z 461.1 [M + H]⁺; ¹H NMR (METHANOL-d₄) δ: 7.26 (d, J = 1.6 |
| | Hz, 1H), 6.22-6.16 (m, 2H), 4.59 (s, 2H), 4.04 (dd, J = 11.6, 4.5 Hz, |
| | 1H), 3.92 (dd, J = 11.6, 4.3 Hz, 1H), 3.83 (td, J = 10.5, 4.5 Hz, 1H), |
| | 3.43-3.33 (m, 1H), 3.32-3.19 (m, 2H), 2.28-2.14 (m, 1H), 1.89 (d, J = |
| | 12.6 Hz, 1H), 0.15 (s, 9H). (3 Exchangeable protons not observed). |
| 318 | MS m/z 434.9 [M + H]⁺; ¹H NMR (METHANOL-d₄) δ: 7.92 (t, J, J = |
| | 57.2 Hz, 1H), 7.43 (s, 1H), 6.35 (d, J, J = 1.7 Hz, 2H), 4.78 (t, J, J = 11.9 |
| | Hz, 2H), 4.32 (s, 1H), 3.13 (s, 1H), 2.23 (s, 1H), 2.17 (s, 3H), 2.00 (d, J, |
| | J = 26.2 Hz, 2H), 1.90 (s, 1H), 1.52 (s, 3H). (4 Exchangeable protons |
| | not observed). |
| 320 | MS m/z 486.1 [M + H]⁺; ¹H NMR (METHANOL-d₄) δ: 8.54 (s, 0.6H, |
| | formic acid), 7.30 (d, J = 5.1 Hz, 1H), 7.08 (d, J = 3.5 Hz, 1H), 6.98 |
| | (dd, J = 5.1, 3.5 Hz, 1H), 6.57 (s, 1H), 5.84 (d, J = 10.8 Hz, 1H), 5.76 |
| | (d, J = 10.8 Hz, 1H), 4.75 (s, 2H), 3.70 (ddd, J = 9.7, 9.3, 9.3 Hz, 1H), |
| | 3.62-3.57 (m, 2H), 3.52 (ddd, J = 10.0, 9.7, 5.9 Hz, 1H), 2.70-2.50 |
| | (m, 5H), 2.25-2.14 (m, 1H), 1.70 (tt, J = 6.9, 6.9 Hz, 2H), 1.59 (m, |
| | 4H). (4 Exchangeable protons not observed). |
| 330 | MS m/z 389.1 [M + H]⁺; ¹H NMR (METHANOL-d₄) δ: 7.44 (s, 1H), |
| | 6.38-6.34 (m, 2H), 4.76 (d, J = 3.8 Hz, 2H), 4.25-4.15 (m, 2H), 4.09 |
| | (d, J = 11.8 Hz, 1H), 3.75-3.64 (m, 2H), 3.59 (td, J = 11.4, 2.5 Hz, |
| | 1H), 3.50-3.43 (m, 1H), 2.27-2.03 (m, 2H). (3 Exchangeable protons |
| | not observed). |
| 331 | MS m/z 434.3 [M + H]⁺; ¹HNMR(400 MHz, METHANOL-d₄) δ 7.93 (t, |
| | J = 57.8 Hz, 1H), 7.43 (s, 1H), 6.35 (d, J = 4.4 Hz, 2H), 4.79 (s, 3H), |
| | 3.72-3.60 (m, 1H), 2.90-2.76 (m, 1H), 2.13 (s, 3H), 2.08-2.0 (m, 1H), |
| | 1.95-1.76 (m, 3H), 1.43-1.28 (m, 3H). (3 exchangeable protons are not |
| | seen) |
| 336 | MS m/z 456.2 [M + H]⁺; ¹H NMR (METHANOL-d₄) δ: 7.55-7.40 (m, |
| | 1H), 6.67 (s, 1H), 6.39-6.29 (m, 2H), 5.81-5.68 (m, 2H), 4.82-4.76 |
| | (m, 2H), 3.76-3.69 (m, 2H), 3.44-3.35 (m, 2H), 3.26 (s, 3H), 2.64 (t, J, |
| | J = 7.0 Hz, 2H), 2.56-2.40 (m, 3H), 2.15-1.97 (m, 1H), 1.91-1.80 (m, |
| | 2H). (3, exchangeable protons not observed). |
| 344 | MS m/z 396.2 [M + H]⁺; ¹H NMR (METHANOL-d₄) δ: 7.37-7.31 (m, |
| | 4H), 7.26 (t, J = 6.4 Hz, 1H), 6.37 (s, 1H), 4.57 (s, 2H), 3.91 (s, 1H), |
| | 3.21-3.13 (m, 1H), 2.96 (td, J = 10.6, 3.9 Hz, 1H), 2.05 (t, J = 12.1 Hz, |
| | 2H), 1.85 (d, J = 9.2 Hz, 2H), 1.67 (d, J = 12.7 Hz, 1H), 1.50-1.28 (m, |
| | 3H) (3, exchangeable protons not observed). |
| 349 | MS m/z 472.2 [M + H]⁺; ¹H NMR (METHANOL-d₄) δ: 7.32 (dd, J = 5.2, |
| | 1.2 Hz, 1H), 7.06-7.00 (m, 1H), 6.98-6.64 (m, 1H), 6.64 (s, 1H), 5.84- |
| | 5.65 (m, 2H), 5.03 (s, 2H), 3.80-3.70 (m, 2H), 3.45-3.32 (m, 2H), |
| | 3.26-3.19 (m, 3H), 2.64 (t, J = 7.0 Hz, 2H), 2.54-2.42 (m, 3H), 2.12- |
| | 2.00 (m, 1H), 1.92-1.81 (m, 2H) (3, exchangeable protons not |
| | observed). |
| 357 | MS m/z 385.9 [M + H]⁺; ¹H NMR (METHANOL-d₄) δ: 7.45 (s, 1H), |
| | 6.60 (s, 1H), 6.36 (s, 1H), 6.32 (s, 1H), 4.53 (s, 2H), 3.91 (s, 1H), 3.15 |
| | (d, J = 11.8 Hz, 1H), 2.94 (s, 1H), 2.06 (d, J = 14.9 Hz, 2H), 1.86 (s, |
| | 2H), 1.66 (d, J = 13.0 Hz, 1H), 1.51-1.42 (m, 2H), 1.33 (s, 1H). (3, |
| | exchangeable protons not observed). |
| 394 | MS m/z 507.2 [M + H]+ 1H NMR (500 MHz, METHANOL-d₄) δ: 8.17- |
| | 8.14 (m, 1H), 8.09-8.06 (m, 1H), 7.34-7.29 (m, 1H), 7.11-7.08 |
| | (m, 1H), 7.04-7.01 (m 1H), 7.00-6.97 (m, 1H), 6.65 (s, 1H), 5.96 (d, |
| | J = 10.0 Hz, 1H), 5.83 (d, J = 10.5 Hz, 1H), 4.78 (s, 2H), 4.47 (td, J = |
| | 10.6, 5.5 Hz, 1H), 4.01 (s, 3H), 3.62 (td, J = 10.6, 5.5 Hz, 1H), 3.08- |
| | 2.97 (m, 1H), 2.79-2.57 (m, 2H), 2.42-2.30 (m, 1H). Trifluoroacetic |
| | acid salt (4 exchangeable protons not seen). |
| 395 | MS m/z 477.2 [M + H]⁺ ¹H NMR (400 MHz, METHANOL-d₄) δ: 8.71 |
| | (d, J = 5.2 Hz, 2H), 7.94 (d, J = 5.1 Hz, 2H), 7.32 (d, J = 5.1 Hz, 1H), |
| | 7.13-7.08 (m, 1H), 7.02-6.96 (m, 1H), 6.68 (s, 1H), 5.95 (d, J = 10.1 |
| | Hz, 1H), 5.83 (d, J = 10.0 Hz, 1H), 4.79 (s, 2H), 4.01-3.92 (m, 1H), |
| | 3.91-3.81 (m, 1H), 2.78-2.64 (m, 3H), 2.44-2.32 (m, 1H). |
| | Trifluoroacetic acid salt (4 exchangeable protons not seen). |
| 430 | MS m/z 413.9 [M + H]⁺; ¹H NMR (METHANOL-d₄) δ: 7.41-7.31 (m, |
| | 2H), 7.16 (t, J = 7.5 Hz, 2H), 6.44 (s, 1H), 4.64 (s, 2H), 3.94 (s, 1H), |
| | 3.24-3.12 (m, 1H), 2.99 (s, 1H), 2.06 (d, J = 13.0 Hz, 2H), 1.89 (s, |
| | 2H), 1.70 (d, J = 12.6 Hz, 1H), 1.43 (dd, J = 45.3, 12.1 Hz, 3H) (3, |
| | exchangeable protons not observed). |
| 447 | MS m/z 511.1 [M + H]+ 1H NMR (400 MHz, METHANOL-d₄) δ: 8.69 |
| | (s, 1H), 8.54 (d, J = 5.0 Hz, 1H), 7.87 (d, J = 5.0 Hz, 1H), 7.32 (d, J = |
| | 5.0 Hz, 1H), 7.13-7.09 (m, 1H), 7.02-6.97 (m, 1H), 6.69 (s, 1H), 5.92 |
| | (d, J = 10.4 Hz, 1H), 5.81 (d, J = 10.4 Hz, 1H), 4.79 (s, 2H), 4.28-4.18 |
| | (m, 1H), 3.84-3.76 (m, 1H), 2.96-2.85 (m, 1H), 2.77-2.63 (m, 2H), |
| | 2.39-2.24 (m, 1H). Trifluoroacetic acid salt (4 exchangeable protons |
| | not seen). |
| 451 | MS m/z 472.2 [M + H]⁺; ¹H NMR (METHANOL-d₄) δ: 7.32 (dd, J = 5.2, |
| | 1.2 Hz, 1H), 7.07-7.01 (m, 1H), 6.67-6.60 (m, 1H), 6.64 (s, 1H), 5.85- |
| | 5.65 (m, 2H), 5.10-4.95 (m, 2H), 3.80-3.70 (m, 2H), 3.45-3.34 (m, |
| | 2H), 3.25-3.20 (m, 3H), 2.70-2.60 (m, 2H), 2.53-2.42 (m, 3H), 2.12- |
| | 1.98 (m, 1H), 1.92-1.81 (m, 2H). (3 exchangeable protons not seen). |
| 452 | MS m/z 456.2 [M + H]⁺; ¹H NMR (METHANOL-d₄) δ: 7.51-7.36 (m, |
| | 1H), 6.74-6.57 (m, 1H), 6.47-6.25 (m, 2H), 5.82-5.62 (m, 2H), 4.79 |
| | (s, 2H), 3.80-3. 65 (m, 2H), 3.45-3.34(m, 2H), 3.27-3.22 (m, 3H), |
| | 2.64 (t, J, J = 7.0 Hz, 2H), 2.53-2.42 (m, 3H), 2.12-2.00 (m, 1H), 1.92- |
| | 1.81 (m, 2H). (3 exchangeable protons not seen). |
| 453 | MS m/z 466.2 [M + H]⁺; ¹H NMR (METHANOL-d₄) δ: 7.38-7.21 (m, |
| | 6H), 6.58-6.52 (m, 1H), 5.78-5.65 (m, 2H), 4.89 (s, 2H), 3.77-3.71 |
| | (m, 2H), 3.24 (s, 3H), 2.66-2.60 (m, 2H), 2.48-2.41 (m, 3H), 2.10- |
| | 1.98 (m, 1H), 1.89-1.82 (m, 2H). (1 exchangeable protons not seen). |
| 454 | MS m/z 486.2 [M + H]⁺; ¹H NMR (METHANOL-d₄) δ: 8.55 (s, 1H), |
| | 7.31 (br s, 1H), 7.07-7.13 (m, 1H), 7.00 (br s, 1H), 6.55 (s, 1H), 5.73- |
| | 5.86 (m, 2H), 4.76 (s, 2H), 3.71-3.81 (m, 3H), 2.65 (s, 5H), 2.38 (br s, |
| | 6H), 1.87 (br t, J = 6.6 Hz, 2H). (2 exchangeable protons not seen). |

### Example 20

### Preparation of Compound 445

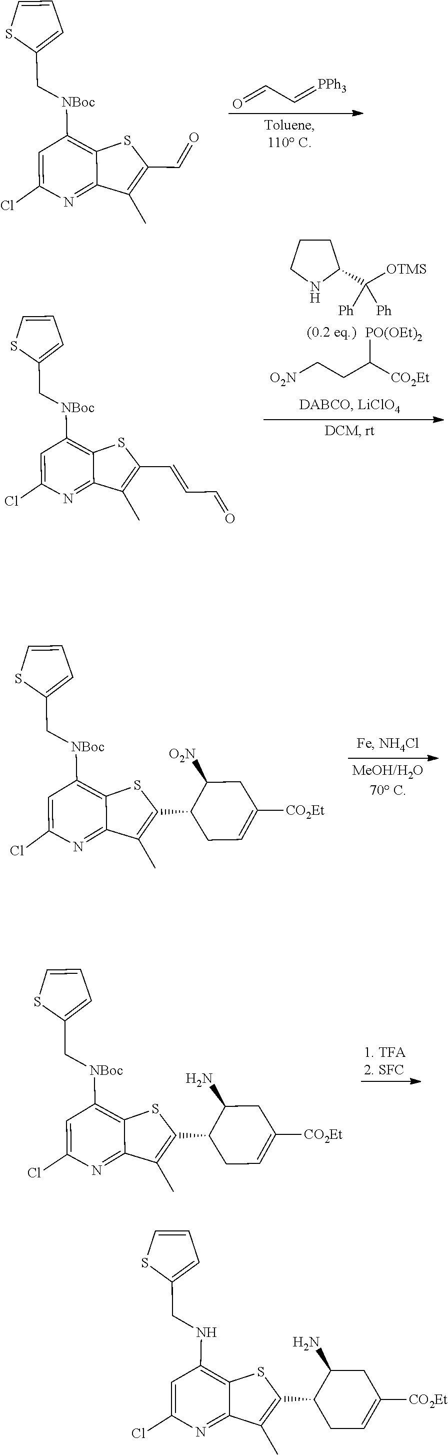

### Step 1: tert-Butyl (E)-(5-chloro-3-methyl-2-(3-oxoprop-1-en-1-yl)thieno[3,2-b]pyridin-7-yl)(thiophen-2-ylmethyl)carbamate

To a solution of tert-butyl (5-chloro-2-formyl-3-methylthieno[3,2-b]pyridin-7-yl)(thiophen-2-ylmethyl)carbamate (6.9 g, 16.3 mmol) in toluene (100 mL) was added (triphenylphosphaneylidene)acetaldehyde (5.46 g, 18 mmol, 1.1 eq.). After stirring at 110° C. for 12 h, the reaction mixture was cooled to room temperature and concentrated in vacuo. The crude residue was purified by silica gel chromatography (DCM/PE 0-60% gradient) to afford tert-butyl (E)-(5-chloro-3-methyl-2-(3-oxoprop-1-en-1-yl)thieno[3,2-b]pyridin-7-yl)(thiophen-2-ylmethyl)carbamate (5.6 g, 76%) as a yellow solid. MS m/z 449.0 [M+H]F.

### Step 2: Ethyl (4S,5S)-4-(7-((tert-butoxycarbonyl)(thiophen-2-ylmethyl)amino)-5-chloro-3-methylthieno[3,2-b]pyridin-2-yl)-5-nitrocyclohex-1-ene-1-carboxylate

To a solution of tert-butyl (E)-(5-chloro-3-methyl-2-(3-oxoprop-1-en-1-yl)thieno[3,2-b]pyridin-7-yl)(thiophen-2-ylmethyl)carbamate (5.6 g, 12.5 mmol) was added ethyl 2-(diethoxyphosphoryl)-4-nitrobutanoate (4.45 g, 15 mmol, 1.2 eq.) (prepared according to the procedure in J. Med. Chem. 2005, 48, 3516-3521), (R)-2-(diphenyl((trimethylsilyl)oxy)methyl)pyrrolidine (812 mg, 2.5 mmol, 0.2 eq.), DABCO (1.4 g, 12.5 mmol, 1.0 eq.) and LiClO₄ (1.33 g, 12.5 mmol, 1.0 eq.). After stirring at room temperature for 48 h, the reaction mixture was concentrated in vacuo. The crude residue was purified by silica gel column chromatography (EA/PE 0-15% gradient) to afford ethyl (4S,5S)-4-(7-((tert-butoxycarbonyl)(thiophen-2-ylmethyl)amino)-5-chloro-3-methylthieno[3,2-b]pyridin-2-yl)-5-nitrocyclohex-1-ene-1-carboxylate (1.1 g, 15%) as a yellow solid. MS m/z 592.0 [M+H]⁺.

### Step 3: ethyl (4S,5S)-5-amino-4-(7-((tert-butoxycarbonyl)(thiophen-2-ylmethyl)amino)-5-chloro-3-methylthieno[3,2-b]pyridin-2-yl)cyclohex-1-ene-1-carboxylate

To a solution of ethyl (4S,5S)-4-(7-((tert-butoxycarbonyl)(thiophen-2-ylmethyl)amino)-5-chloro-3-methylthieno[3,2-b]pyridin-2-yl)-5-nitrocyclohex-1-ene-1-carboxylate (500 mg, 844 mmol) in MeOH (18 mL) and H₂O (2 mL) was added Fe powder (472 mg, 8.4 mol, 10 eq.) and NH₄Cl (903 mg, 17 mmol, 2.0 eq.). After stirring the reaction mixture at 70° C. for 12 h, the reaction mixture was cooled to room temperature and filtered through a pad of Celite, washing with MeOH (50 mL×2). The filtrate was concentrated to afford crude ethyl (4S,5S)-5-amino-4-(7-((tert-butoxycarbonyl)(thiophen-2-ylmethyl)amino)-5-chloro-3-methylthieno[3,2-b]pyridin-2-yl)cyclohex-1-ene-1-carboxylate (450 mg, 95%) as a yellow solid that was used without further purification. MS m/z 562.0 [M+H]⁺.

### Step 4: Ethyl (4S,5S)-5-amino-4-(5-chloro-3-methyl-7-((thiophen-2-ylmethyl)amino)thieno[3,2-b]pyridin-2-yl)cyclohex-1-ene-1-carboxylate

To a solution of ethyl (4S,5S)-5-amino-4-(7-((tert-butoxycarbonyl)(thiophen-2-ylmethyl)amino)-5-chloro-3-methylthieno[3,2-b]pyridin-2-yl)cyclohex-1-ene-1-carboxylate (450 mg, 800 mmol) in DCM (5 mL) was added TFA (2 mL). After stirring at room temperature for 2 h, the reaction mixture was concentrated in vacuo and purified by reverse phase silica gel chromatography (MeCN/H₂O 5-95% gradient) followed by prep-SFC (Dr.Maish Reprosil Chiral-AM, Supercritical CO₂/MeOH (+0.1% 7.0 mol/l NH₃ in MeOH)/MeOH) to afford ethyl (4S,5S)-5-amino-4-(7-((tert-butoxycarbonyl)(thiophen-2-ylmethyl)amino)-5-chloro-3-methylthieno[3,2-b]pyridin-2-yl)cyclohex-1-ene-1-carboxylate (35 mg, 10%) as a white solid. MS m/z 462.0 [M+H]⁺;]⁺; ¹H NMR (METHANOL-d₄) δ: 7.34-7.25 (m, 1H), 7.09-7.01 (m, 2H), 6.99-6.94 (m, 1H), 6.58-6.49 (m, 1H), 4.72 (s, 2H), 4.26-4.12 (m, 2H), 3.40-3.34 (m, 2H), 2.91-2.82 (m, 1H), 2.80-2.65 (m, 1H), 2.59-2.49 (m, 1H), 2.45-2.25 (m, 4H), 1.33-1.25 (m, 3H). (3 Exchangeable protons not observed).

The compounds below were prepared according to the procedure of Example 20 by substituting the appropriate starting materials, reagents, and reaction conditions, and followed by chiral SFC resolution if needed.

| Compound | Spectral Data |
|---|---|
| 277 | MS m/z 462.0 [M + H]⁺; ]⁺; ¹H NMR (METHANOL-d₄) δ: 7.34-7.25 (m, |
| | 1H), 7.09-7.01 (m, 2H), 6.99-6.94 (m, 1H), 6.58-6.49 (m, 1H), 4.72 |
| | (s, 2H), 4.26-4.12 (m, 2H), 3.40-3.34 (m, 2H), 2.91-2.82 (m, 1H), |
| | 2.80-2.65 (m, 1H), 2.59-2.49 (m, 1H), 2.45-2.25 (m, 4H), 1.33-1.25 (m, |
| | 3H) (3 Exchangeable protons not observed). |

### Example 21

### Preparation of Compound 446

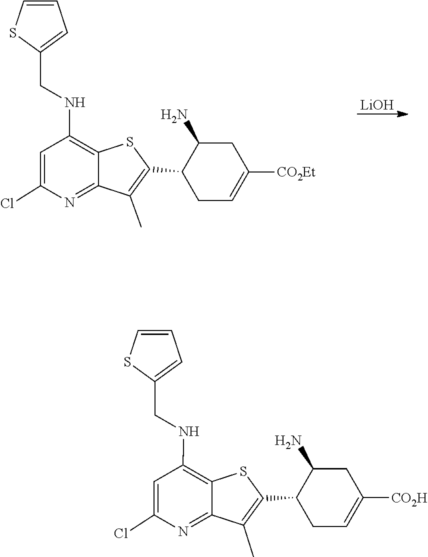 [SMILES: CCOC(=O)C1=CC[C@H](c2sc3c(NCc4cccs4)cc(Cl)nc3c2C)[C@@H](N)C1 ; "LiOH" ; Cc1c([C@H]2CC=C(C(=O)O)C[C@@H]2N)sc2c(NCc3cccs3)cc(Cl)nc12]

### Step 1: (4S,5S)-5-Amino-4-(5-chloro-3-methyl-7-((thiophen-2-ylmethyl)amino)thieno[3,2-b]pyridin-2-yl)cyclohex-1-ene-1-carboxylic acid

Ethyl (4S,5S)-5-amino-4-(5-chloro-3-methyl-7-((thiophen-2-ylmethyl)amino)thieno[3,2-b]pyridin-2-yl)cyclohex-1-ene-1-carboxylate (50 mg, 0.1 mmol) (prepared in Example 20, step 4) was hydrolyzed according to the procedure described in Example 22, step 2 to afford (4S,5S)-5-amino-4-(5-chloro-3-methyl-7-((thiophen-2-ylmethyl)amino)thieno[3,2-b]pyridin-2-yl)cyclohex-1-ene-1-carboxylic acid (30 mg, 60%) as white solid. MS m/z 433.9 [M+H]⁺; ¹H NMR (METHANOL-d₄) δ: 7.35-7.25 (m, 1H), 7.18-7.05 (m, 2H), 7.05-6.95 (m, 1H), 6.54 (s, 1H), 4.75 (s, 2H), 4.60 (s, 2H), 3.82-3.65 (m, 2H), 3.05-2.97 (m, 1H), 2.90-2.87 (m, 1H), 2.75-2.68 (m, 1H), 2.62-2.54 (m, 1H), 2.40 (s, 3H).

### Example 22

### Preparation of Compound 274

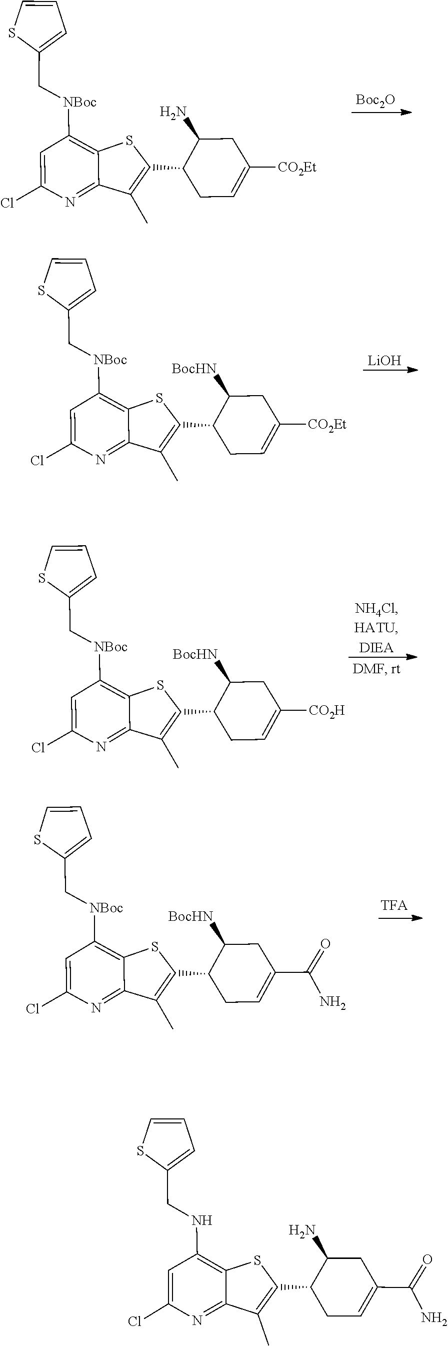

### Step 1: Ethyl (4S,5S)-4-(7-((tert-butoxycarbonyl)(thiophen-2-ylmethyl)amino)-5-chloro-3-methylthieno[3,2-b]pyridin-2-yl)-5-((tert-butoxycarbonyl)amino)cyclohex-1-ene-1-carboxylate

To a solution of ethyl (4S,5S)-5-amino-4-(7-((tert-butoxycarbonyl)(thiophen-2-ylmethyl)amino)-5-chloro-3-methylthieno[3,2-b]pyridin-2-yl)cyclohex-1-ene-1-carboxylate (280 mg, 0.5 mmol) (prepared according to Example 20) in THF (5 mL) was added Boc₂O (217 mg, 1.0 mmol, 2 eq.) and TEA (151 mg, 1.5 mmol, 3 eq.). After stirring at room temperature for 12 h, the reaction mixture was concentrated in vacuo and the crude residue was purified by silica gel column chromatography (EA/PE 0-15% gradient) to afford ethyl (4S,5S)-4-(7-((tert-butoxycarbonyl)(thiophen-2-ylmethyl)amino)-5-chloro-3-methylthieno[3,2-b]pyridin-2-yl)-5-((tert-butoxycarbonyl)amino)cyclohex-1-ene-1-carboxylate (320 mg, 98%) as a white solid. MS m/z 661.9 [M+H]⁺.

### Step 2: (4S,5S)-4-(7-((tert-Butoxycarbonyl)(thiophen-2-ylmethyl)amino)-5-chloro-3-methylthieno[3,2-b]pyridin-2-yl)-5-((tert-butoxycarbonyl)amino)cyclohex-1-ene-1-carboxylic acid

To a solution of ethyl (4S,5S)-4-(7-((tert-butoxycarbonyl)(thiophen-2-ylmethyl)amino)-5-chloro-3-methylthieno[3,2-b]pyridin-2-yl)-5-((tert-butoxycarbonyl)amino)cyclohex-1-ene-1-carboxylate (290 mg, 0.44 mmol) in a mixture of THF (4 mL), MeOH (1 mL), and H₂O (1 mL) was added lithium hydroxide monohydrate (92 mg, 2.2 mmol, 5 eq.). After stirring at room temperature for 3 h, the reaction mixture was diluted with H₂O (20 mL), acidified to pH 5 with 1M HCl, and extracted with EtOAc (20 mL×2). The combined organics were washed with brine (20 mL×2), dried over Na₂SO₄, filtered and concentrated in vacuo to afford crude (4S,5S)-4-(7-((tert-butoxycarbonyl)(thiophen-2-ylmethyl)amino)-5-chloro-3-methylthieno[3,2-b]pyridin-2-yl)-5-((tert-butoxycarbonyl)amino)cyclohex-1-ene-1-carboxylic acid (260 mg, 94%) as a white solid. MS m/z 634.0 [M+H]⁺.

### Step 3: tert-Butyl (2-((1S,6S)-6-((tert-butoxycarbonyl)amino)-4-carbamoylcyclohex-3-en-1-yl)-5-chloro-3-methylthieno[3,2-b]pyridin-7-yl)(thiophen-2-ylmethyl)carbamate

To a solution of (4S,5S)-4-(7-((tert-butoxycarbonyl)(thiophen-2-ylmethyl)amino)-5-chloro-3-methylthieno[3,2-b]pyridin-2-yl)-5-((tert-butoxycarbonyl)amino)cyclohex-1-ene-1-carboxylic acid (100 mg, 0.16 mmol) in DMF (2 mL) was added HATU (73 mg, 0.19 mmol, 1.2 eq.), NH₄Cl (42 mg, 0.8 mmol, 5 eq.), and DIEA (102 mg, 0.8 mmol, 1.2 eq.). After stirring at room temperature for 12 h, the reaction mixture was diluted with H₂O (30 mL) and extracted with EtOAc (30 mL×2). The combined organics were washed with brine (30 mL×2), dried over Na₂SO₄, filtered, and concentrated in vacuo to afford crude tert-butyl (2-((1S,6S)-6-((tert-butoxycarbonyl)amino)-4-carbamoylcyclohex-3-en-1-yl)-5-chloro-3-methylthieno[3,2-b]pyridin-7-yl)(thiophen-2-ylmethyl)carbamate (99 mg, 99%) as a yellow oil that was used without further purification. MS m/z 633.1 [M+H]⁺.

### Step 4: (4S,5S)-5-Amino-4-(5-chloro-3-methyl-7-((thiophen-2-ylmethyl)amino)thieno[3,2-b]pyridin-2-yl)cyclohex-1-ene-1-carboxamide

tert-Butyl (2-((1S,6S)-6-((tert-butoxycarbonyl)amino)-4-carbamoylcyclohex-3-en-1-yl)-5-chloro-3-methylthieno[3,2-b]pyridin-7-yl)(thiophen-2-ylmethyl)carbamate (100 mg, 0.16 mmol) was deprotected according to the procedure described in Example 5, step 2 to afford (4S,5S)-5-amino-4-(5-chloro-3-methyl-7-((thiophen-2-ylmethyl)amino)thieno[3,2-b]pyridin-2-yl)cyclohex-1-ene-1-carboxamide (54 mg, 79%) as awhite solid. MS m/z 433.1 [M+H]⁺; ¹H NMR (METHANOL-d₄) δ: 7.29 (t, J=4.9 Hz, 1H), 7.07 (t, J=5.8 Hz, 1H), 6.97 (dd, J=8.4, 4.6 Hz, 1H), 6.80 (d, J=29.9 Hz, 1H), 6.61 (d, J=5.4 Hz, 1H), 4.76 (d, J=11.6 Hz, 2H), 3.91-3.60 (m, 2H), 3.06-2.45 (m, 4H), 2.42 (d, J=7.7 Hz, 3H).

The compound below was prepared according to the procedure of Example 22 by substituting the appropriate starting materials, reagents, reaction conditions, and followed by chiral SFC resolution if needed.

| Compound | Spectral Data |
|---|---|
| 312 | MS m/z 460.9 [M + H]⁺; ¹H NMR (400 MHz, METHANOL-d₄) δ: 7.32- |
| | 7.27 (m, 1H), 7.10-7.08 (m, 1H), 7.00-6.95 (m, 1H), 6.65-6.60 |
| | (m, 1H), 6.06 (s, 1H), 4.78 (s, 2H), 3.80-3.65(m, 2H), 3.25-3.10 (m, |
| | 3H), 3.00 (s, 3H), 2.85-2.73 (m, 2H), 2.48-2.72 (m, 2H), 2.42 (s, 3H). |
| | (3 exchangeable protons are not seen) |
| 313 | MS m/z 503.1 [M + H]⁺; ¹H NMR (400 MHz, METHANOL-d₄) δ: 7.30 |
| | (d, J = 5.1 Hz, 1H), 7.08 (s, 1H), 6.97 (t, J = 4.2 Hz, 1H), 6.61 (s, |
| | 1H), 6.09 (d, J = 17.1 Hz, 1H), 4.77 (s, 2H), 4.13-3.72 (m, 2H), 2.87- |
| | 2.60 (m, 3H), 2.52 (s, 1H), 2.41 (s, 3H). (3 exchangeable protons are not |
| | seen) |
| 353 | MS m/z 508.9 [M + H]⁺; ¹H NMR (400 MHz, METHANOL-d₄) δ: 7.64- |
| | 7.57 (m, 2H), 7.36-7.25 (m, 3H), 7.16-7.03 (m, 2H), 6.99-6.89 |
| | (m, 1H), 6.82 (s, 1H), 6.60-6.58 (m, 1H), 4.77-4.72 (m, 2H), 3.81- |
| | 3.65 (m, 2H), 3.15-3.06 (m, 1H), 2.90-2.87 (m, 1H), 2.79-2.68 (m, |
| | 1H), 2.63-2.51 (m, 1H), 2.45-2.40 (m, 3H (3 exchangeable protons |
| | are not seen) |

### Example 23

### Preparation of Compound 246

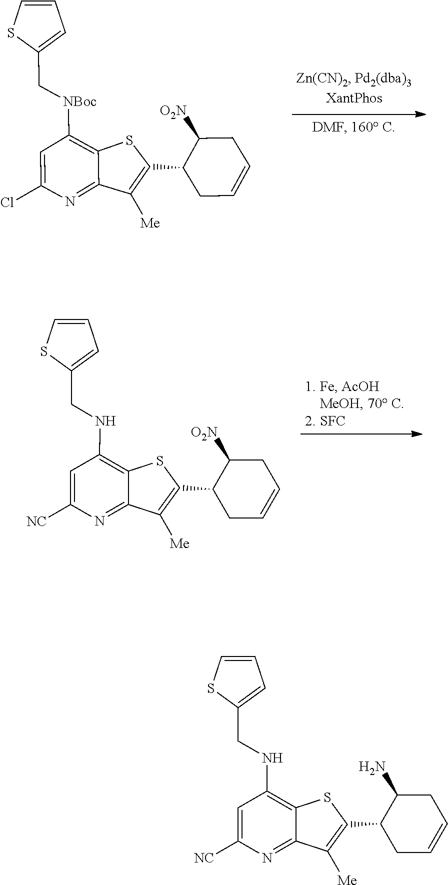 [SMILES: Cc1c([C@H]2CC=CC[C@@H]2[N+](=O)[O-])sc2c(N(Cc3cccs3)C(=O)OC(C)(C)C)cc(Cl)nc12 ; Cc1c([C@H]2CC=CC[C@@H]2[N+](=O)[O-])sc2c(NCc3cccs3)cc(C#N)nc12 ; Cc1c([C@H]2CC=CC[C@@H]2N)sc2c(NCc3cccs3)cc(C#N)nc12]

### Step 1: 3-Methyl-2-((1S,6S)-6-nitrocyclohex-3-en-1-yl)-7-((thiophen-2-ylmethyl)amino)thieno[3,2-b]pyridine-5-carbonitrile

A mixture of tert-butyl (5-chloro-3-methyl-2-((1S,6S)-6-nitrocyclohex-3-en-1-yl)thieno[3,2-b]pyridin-7-yl)(thiophen-2-ylmethyl)carbamate (prepared according to the procedures described in Example 13) (1.0 g, 0.4 mmol), zinc cyanide (900 mg, 1.5 mmol, 4 eq.), 4,5-bis(diphenylphosphino)-9,9-dimethylxanthene (110 mg, 0.2 mmol, 0.2 eq.), tris(dibenzylideneacetone)dipalladium (88 mg, 0.1 mmol, 0.2 eq.), and DMF (10 mL) was heated at 160° C. for 1 h in a microwave reactor. The reaction mixture was allowed to cool to room temperature and diluted with water (50 mL). The aqueous solution was extracted with EtOAc (50 mL×2). The combined organics were washed with brine (2×50 mL), dried over Na₂SO₄, filtered, and concentrated in vacuo. The crude residue was purified by silica gel column chromatography (EA/Hex 0-50% gradient) to afford 3-methyl-2-((1S,6S)-6-nitrocyclohex-3-en-1-yl)-7-((thiophen-2-ylmethyl)amino)thieno[3,2-b]pyridine-5-carbonitrile (280 mg, 35%) as a white solid. MS m/z 411.1 [M+H]⁺.

### Step 2: tert-Butyl (2-((1S,6S)-6-aminocyclohex-3-en-1-yl)-3-chloro-5-cyanothieno[3,2-b]pyridin-7-yl)(furan-2-ylmethyl)carbamate

According to the procedure described in step 4 of Example 2, 3-methyl-2-((1S,6S)-6-nitrocyclohex-3-en-1-yl)-7-((thiophen-2-ylmethyl)amino)thieno[3,2-b]pyridine-5-carbonitrile (250 mg, 0.6 mmol) was reduced to followed by SFC separation (using conditions described in Example 1) to afford 3-methyl-2-((1S,6S)-6-nitrocyclohex-3-en-1-yl)-7-((thiophen-2-ylmethyl)amino)thieno[3,2-b]pyridine-5-carbonitrile (70 mg, 30%) as a white solid. MS m/z 381.2 [M+H]⁺; ¹H NMR (400 MHz, METHANOL-d₄) δ: 7.32-7.26 (m, 1H), 7.13-7.05 (m, 1H), 7.01-6.94 (m, 1H), 6.91 (s, 1H), 5.82-5.70 (m, 2H), 4.80 (s, 2H), 3.41-3.32 (m, 1H), 3.22-3.11 (m, 1H), 2.55-2.31 (m, 6H), 2.20-2.00 (m, 1H).

The compound below was prepared according to the procedure of Example 23 by substituting the appropriate starting materials, reagents, reaction conditions, and followed by chiral SFC resolution if needed.

| Compound | Spectral Data |
|---|---|
| 242 | MS m/z 385.1 [M + H]⁺; ¹H NMR (400 MHz, METHANOL-d₄) δ: 7.47- |
| | 7.44 (m, 1H), 7.11-7.03 (m, 1H), 6.38-6.34 (m, 2H), 5.87-5.72 |
| | (m, 2H), 4.62-4.57 (m, 2H), 3.60-3.49 (m, 1H), 3.39-3.32 (m, 1H), |
| | 2.59-2.42 (m, 3H), 2.22-2.11 (m, 1H). (3 exchangeable protons are |
| | not seen) |
| 282 | MS m/z 365.0 [M + H]⁺; ¹H NMR (400 MHz, METHANOL-d₄) δ: 7.46- |
| | 7.38 (m, 2H), 6.42-6.27 (m, 2H), 5.86-5.68 (m, 2H), 4.80-4.71 |
| | (m, 2H), 3.41-3.32 (m, 1H), 3.24-3.11 (m, 1H), 2.56-2.24 (m, 6H), |
| | 2.20-2.00 (m, 1H). (3 exchangeable protons are not seen) |
| 289 | MS m/z 365.1 [M + H]⁺; ¹H NMR (400 MHz, METHANOL-d₄) δ: 7.46- |
| | 7.38 (m, 2H), 6.42-6.27 (m, 2H), 5.86-5.68 (m, 2H), 4.80-4.71 |
| | (m, 2H), 3.41-3.32 (m, 1H), 3.24-3.11 (m, 1H), 2.56-2.24 (m, 6H), |
| | 2.20-2.00 (m, 1H). (3 exchangeable protons are not seen) |
| 443 | MS m/z 385.1 [M + H]⁺; ¹H NMR (400 MHz, METHANOL-d₄) δ: 7.47- |
| | 7.44 (m, 1H), 7.11-7.03 (m, 1H), 6.38-6.34 (m, 2H), 5.87-5.72 |
| | (m, 2H), 4.62-4.57 (m, 2H), 3.60-3.49 (m, 1H), 3.39-3.32 (m, 1H), |
| | 2.59-2.42 (m, 3H), 2.22-2.11 (m, 1H). (3 exchangeable protons are |
| | not seen) |

### Example 24

### Preparation of Compound 199

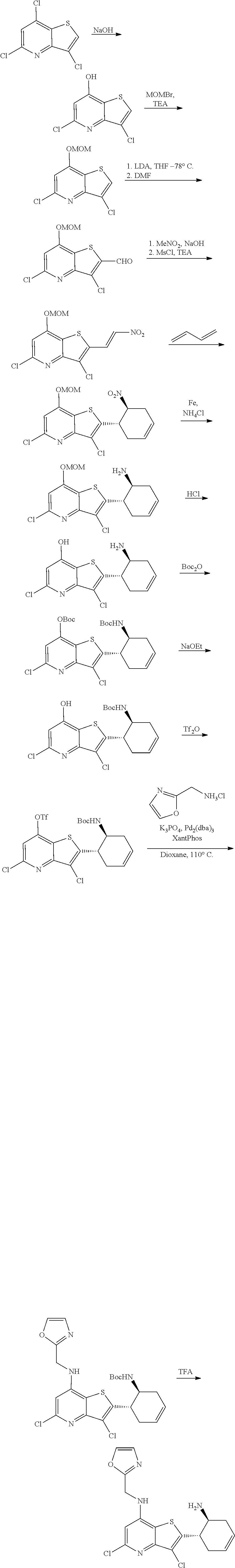

### Step 1: 3,5-Dichlorothieno[3,2-b]pyridin-7-ol

To a solution of 3,5,7-trichlorothieno[3,2-b]pyridine (30 g, 126 mmol) in a mixture of DMSO (300 mL) and H₂O (60 mL) was added NaOH (12.6 g, 314 mmol, 2.5 eq.). After stirring at 110° C. for 12 h, the reaction mixture was cooled to room temperature, diluted with H₂O (300 mL) and acidified to pH 5 with conc. HCl. The mixture was filtered to give 3,5-dichlorothieno[3,2-b]pyridin-7-ol (27 g, 98%) as a yellow solid that was used without further purification. MS m/z 219.8 [M+H]⁺.

### Step 2: 3,5-Dichloro-7-(methoxymethoxy)thieno[3,2-b]pyridine

To a solution cooled of 3,5-dichlorothieno[3,2-b]pyridin-7-ol (27 g, 123 mmol) and TEA (85.3 mL, 613 mmol, 5 eq.) in DMF (200 mL), at 0° C., was added MOMBr (30 mL, 368 mmol, 3 eq.) dropwise. After stirring at room temperature for 2 h, the reaction mixture was diluted with EtOAc (500 mL) and washed with water (500 mL×5) and brine (500 mL×2). The organic solution was dried over Na₂SO₄, filtered, and concentrated in vacuo. The crude solid was purified by silica gel column chromatography (EA/PE 0-20% gradient) to afford 3,5-dichloro-7-(methoxymethoxy)thieno[3,2-b]pyridine (20 g, 62%) as a yellow solid. MS m/z 263.8 [M+H]⁺.

### Step 3: 3,5-Dichloro-7-(methoxymethoxy)thieno[3,2-b]pyridine-2-carbaldehyde

According to the procedure described in step 1 of Intermediate 1, 3,5-dichloro-7-(methoxymethoxy)thieno[3,2-b]pyridine (20 g, 76 mmol) was used to afford crude 3,5-dichloro-7-(methoxymethoxy)thieno[3,2-b]pyridine-2-carbaldehyde (22.1 g, 99%) as a yellow solid. MS m/z 291.8 [M+H]⁺.

### Step 4: (E)-3,5-Dichloro-7-(methoxymethoxy)-2-(2-nitrovinyl)thieno[3,2-b]pyridine

According to the procedure described in steps 2 and 3 of Intermediate 1, 3,5-dichloro-7-(methoxymethoxy)thieno[3,2-b]pyridine-2-carbaldehyde (22 g, 75 mmol) was used to afford (E)-3,5-dichloro-7-(methoxymethoxy)-2-(2-nitrovinyl)thieno[3,2-b]pyridine (21 g, 83%) as a yellow solid. MS m/z 335.2 [M+H]⁺.

### Step 5: 3,5-Dichloro-7-(methoxymethoxy)-2-((1S,6S)-6-nitrocyclohex-3-en-1-yl)thieno[3,2-b]pyridine

According to the procedure described in step 1 of Example 13, 3,5-dichloro-7-(methoxymethoxy)-2-((1S,6S)-6-nitrocyclohex-3-en-1-yl)thieno[3,2-b]pyridine (7 g, 21 mmol) was used to afford crude (E)-3,5-dichloro-7-(methoxymethoxy)-2-(2-nitrovinyl)thieno[3,2-b]pyridine (23 g, 99%) as a yellow oil. MS m/z 388.8 [M+H]⁺.

### Step 6: (1S,6S)-6-(3,5-Dichloro-7-(methoxymethoxy)thieno[3,2-b]pyridin-2-yl)cyclohex-3-en-1-amine

According to the procedure described in step 2 of Example 13, 3,5-dichloro-7-(methoxymethoxy)-2-((1S,6S)-6-nitrocyclohex-3-en-1-yl)thieno[3,2-b]pyridine (23 g, 59 mmol) was used to afford crude (1S,6S)-6-(3,5-dichloro-7-(methoxymethoxy)thieno[3,2-b]pyridin-2-yl)cyclohex-3-en-1-amine (20 g, 94%) as a yellow solid. MS m/z 359.0 [M+H]⁺.

### Step 7: 2-((1S,6S)-6-Aminocyclohex-3-en-1-yl)-3,5-dichlorothieno[3,2-b]pyridin-7-ol

To a solution of crude (1S,6S)-6-(3,5-dichloro-7-(methoxymethoxy)thieno[3,2-b]pyridin-2-yl)cyclohex-3-en-1-amine (20 g, 56 mmol) in EtOAc (200 mL) was added 4M HCl (solution in dioxane, 20 mL). After stirring at room temperature for 2 h, the reaction mixture was concentrated in vacuo to afford crude 2-((1S,6S)-6-aminocyclohex-3-en-1-yl)-3,5-dichlorothieno[3,2-b]pyridin-7-ol (17 g, 97%) as a yellow solid which was used without further purification. MS m/z 315.0 [M+H]⁺.

### Step 8: tert-Butyl ((1S,6S)-6-(7-((tert-butoxycarbonyl)oxy)-3,5-dichlorothieno[3,2-b]pyridin-2-yl)cyclohex-3-en-1-yl)carbamate

To a solution of crude 2-((1S,6S)-6-aminocyclohex-3-en-1-yl)-3,5-dichlorothieno[3,2-b]pyridin-7-ol (17 g, 28 mmol) in THF (30 mL) was added TEA (20 mL, 141 mmol, 5 eq.) and Boc₂O (12.3 g, 57 mmol, 2 eq.). After stirring at room temperature for 12 h, the reaction mixture was concentrated in vacuo and the crude residue was purified by silica gel column chromatography (EA/PE 0-15% gradient) to afford tert-butyl ((1S,6S)-6-(7-((tert-butoxycarbonyl)oxy)-3,5-dichlorothieno[3,2-b]pyridin-2-yl)cyclohex-3-en-1-yl)carbamate (9.5 g, 48%) as a yellow oil. MS m/z 414.8 [M-Boc+H]⁺.

### Step 9: tert-Butyl ((1S,6S)-6-(3,5-dichloro-7-hydroxythieno[3,2-b]pyridin-2-yl)cyclohex-3-en-1-yl)carbamate

To a solution of tert-butyl ((1S,6S)-6-(7-((tert-butoxycarbonyl)oxy)-3,5-dichlorothieno[3,2-b]pyridin-2-yl)cyclohex-3-en-1-yl)carbamate (9.5 g, 18 mmol) in MeOH (20 mL) was added NaOEt (2.5 g, 37 mmol, 2 eq.). After stirring at room temperature for 2 h, the reaction mixture was concentrated in vacuo to afford crude tert-butyl ((1S,6S)-6-(3,5-dichloro-7-hydroxythieno[3,2-b]pyridin-2-yl)cyclohex-3-en-1-yl)carbamate (4.5 g, 59%) as a yellow solid which was used without further purification. MS m/z 414.8 [M+H]⁺.

### Step 10: 2-((1S,6S)-6-((tert-Butoxycarbonyl)amino)cyclohex-3-en-1-yl)-3,5-dichlorothieno[3,2-b]pyridin-7-yl trifluoromethanesulfonate

To a solution of crude 2-((1S,6S)-6-((tert-butoxycarbonyl)amino)cyclohex-3-en-1-yl)-3,5-dichlorothieno[3,2-b]pyridin-7-yl trifluoromethanesulfonate (3.0 g, 7.2 mmol) in pyridine (30 mL), cooled to 0° C., was added Tf₂O (1.8 mL, 10.8 mmol, 1.5 eq.) dropwise. After stirring at room temperature for 2 h, the reaction mixture was diluted with EtOAc (100 mL) and washed with water (100 mL×5) and brine (200 mL×2). The organics were dried over Na₂SO₄, filtered, and concentrated in vacuo. The crude residue was purified by silica gel column chromatography (EA/PE 0-10% gradient) to afford 2-((1S,6S)-6-((tert-butoxycarbonyl)amino)cyclohex-3-en-1-yl)-3,5-dichlorothieno[3,2-b]pyridin-7-yl trifluoromethanesulfonate (2.5 g, 63%) as a white solid. MS m/z 546.8 [M+H]⁺.

### Step 11: tert-Butyl ((1S,6S)-6-(3,5-dichloro-7-((oxazol-2-ylmethyl)amino)thieno[3,2-b]pyridin-2-yl)cyclohex-3-en-1-yl)carbamate

A mixture of 2-((1S,6S)-6-((tert-butoxycarbonyl)amino)cyclohex-3-en-1-yl)-3,5-dichlorothieno[3,2-b]pyridin-7-yl trifluoromethanesulfonate (500 mg, 0.9 mmol), oxazol-2-ylmethanamine hydrogen chloride (148 mg, 1.1 mmol, 1.2 eq.), K₃PO₄ (156 mg, 4 mmol, 4 eq.), 4,5-bis(diphenylphosphino)-9,9-dimethylxanthene (22 mg, 0.02 mmol, 0.2 eq.), tris(dibenzylideneacetone)dipalladium (17 mg, 0.01 mmol, 0.1 eq.), and dioxane (10 mL) was was stirred 110° C. for 12 h. The reaction mixture was cooled to room temperature and concentrated in vacuo. The crude residue was purified by silica gel column chromatography to afford tert-butyl ((1S,6S)-6-(3,5-dichloro-7-((oxazol-2-ylmethyl)amino)thieno[3,2-b]pyridin-2-yl)cyclohex-3-en-1-yl)carbamate (200 mg, 44%) as a yellow oil. MS m/z 494.9 [M+H]⁺.

### Step 12: 2-((1S,6S)-6-Aminocyclohex-3-en-1-yl)-3,5-dichloro-N-(oxazol-2-ylmethyl)thieno[3,2-b]pyridin-7-amine

tert-Butyl ((1S,6S)-6-(3,5-dichloro-7-((oxazol-2-ylmethyl)amino)thieno[3,2-b]pyridin-2-yl)cyclohex-3-en-1-yl)carbamate (200 mg, 0.4 mmol) was deprotected according to the procedure described in Example 5, step 2 to afford 2-((1S,6S)-6-aminocyclohex-3-en-1-yl)-3,5-dichloro-N-(oxazol-2-ylmethyl)thieno[3,2-b]pyridin-7-amine (33 mg, 21%) as a white solid. MS m/z 395.1 [M+H]⁺; ¹H NMR (METHANOL-d₄) δ: 7.95-6.85 (m, 1H), 7.24-7.10 (m, 1H), 6.70-6.59 (m, 1H), 5.85-5.70 (m, 2H), 4.65 (s, 2H), 3.59-3.50 (m, 1H), 3.28-3.14 (m, 1H), 2.60-2.38 (m, 3H), 2.30-2.05 (m, 1H).

### Example 25

### Preparation of Compound 393

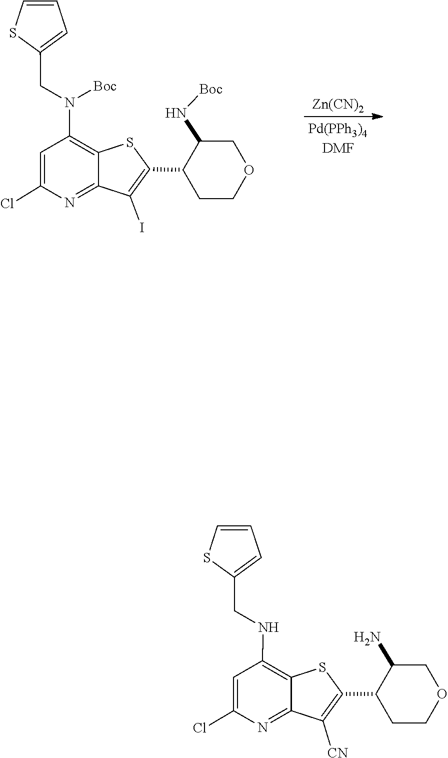 [SMILES: CC(C)(C)OC(=O)N[C@H]1COCC[C@@H]1c1sc2c(N(Cc3cccs3)C(=O)OC(C)(C)C)cc(Cl)nc2c1I ; "Zn(CN)2" ; N#Cc1c([C@H]2CCOC[C@@H]2N)sc2c(NCc3cccs3)cc(Cl)nc12]

### Step 1: rac-2-((3R,4S)-3-Aminotetrahydro-2H-pyran-4-yl)-5-chloro-7-((thiophen-2-ylmethyl)amino)thieno[3,2-b]pyridine-3-carbonitrile

An oven-dried, 8-mL reaction vial was charged with Pd(PPh₃)₄ (11.6 mg, 0.01 mmol, 0.1 eq), Zinc Cyanide (7 mg, 0.06 mmol, 0.6 eq) and rac-tert-butyl (2-((3R,4S)-3-((tert-butoxycarbonyl)amino)tetrahydro-2H-pyran-4-yl)-5-chloro-3-iodothieno[3,2-b]pyridin-7-yl)(thiophen-2-ylmethyl)carbamate (70.6 mg, 0.1 mmol) which was prepared by following the procedure in Example 11. DMF (1.0 mL) was added to the mixture and the solution was degassed with argon. The sealed vial was heated at 100° C. overnight. After cooling, 0.2 mL of NH₄Cl (sat. aq.) and 0.2 mL of NaHCO₃ (sat. aq.) were added. The mixture was filtered and washed with DCM (0.5 mL×3). The combined filtrate was concentrated and purified by preparative HPLC eluting with 10-100% ACN in water with 0.1% TFA to provide rac-2-((3R,4S)-3-aminotetrahydro-2H-pyran-4-yl)-5-chloro-7-((thiophen-2-ylmethyl)amino)thieno[3,2-b]pyridine-3-carbonitrile (16.2 mg, 40% yield). MS m/z 405.1 [M+H]⁺; ¹H NMR (METHANOL-d₄) δ: 7.32 (d, J=5.1 Hz, 1H), 7.11 (d, J=3.5 Hz, 1H), 6.99 (dd, J=5.1, 3.5 Hz, 1H), 6.70 (s, 1H), 4.79 (s, 2H), 4.08 (dd, J=12.7, 3.6 Hz, 1H), 4.03-3.96 (m, 1H), 3.93-3.84 (m, 1H), 3.84-3.78 (m, 1H), 3.75 (dt, J=10.0, 5.2 Hz, 1H), 3.64 (ddd, J=12.2, 9.1, 4.0 Hz, 1H), 2.00-1.85 (m, 2H). (3 Exchangeable protons not observed).

### Example 26

### Preparation of Compound 324

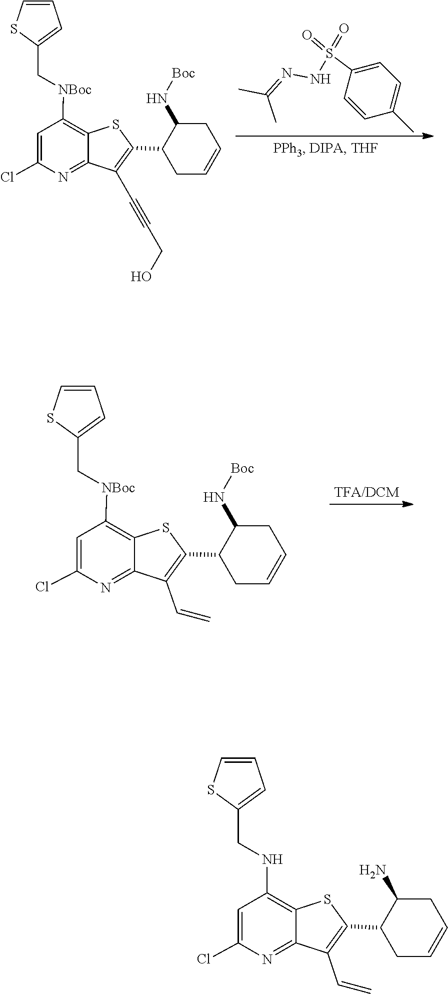 [SMILES: CC(C)(C)OC(=O)N[C@H]1CC=CC[C@@H]1c1sc2c(N(Cc3cccs3)C(=O)OC(C)(C)C)cc(Cl)nc2c1C#CCO ; CC(C)=NNS(=O)(=O)c1ccc(C)cc1 ; C=Cc1c([C@H]2CC=CC[C@@H]2NC(=O)OC(C)(C)C)sc2c(N(Cc3cccs3)C(=O)OC(C)(C)C)cc(Cl)nc12 ; C=Cc1c([C@H]2CC=CC[C@@H]2N)sc2c(NCc3cccs3)cc(Cl)nc12]

### Step 1: rac-tert-Butyl (2-((1S,6S)-6-((tert-butoxycarbonyl)amino)cyclohex-3-en-1-yl)-5-chloro-3-(propa-1,2-dien-1-yl)thieno[3,2-b]pyridin-7-yl)(thiophen-2-ylmethyl)carbamate

An oven-dried, 8-mL reaction vial was charged with PPh₃ (39.3 mg, 0.15 mmol, 1.5 eq), N-isopropylidene-N′-2-nitrobenzenesulfonyl hydrazine (IPNBSH) (33.9 mg, 0.15 mmol, 1.5 eq) and rac-tert-butyl (2-((1S,6S)-6-((tert-butoxycarbonyl)amino)cyclohex-3-en-1-yl)-5-chloro-3-(3-hydroxyprop-1-yn-1-yl)thieno[3,2-b]pyridin-7-yl)(thiophen-2-ylmethyl)carbamate (63.0 mg, 0.1 mmol) which was prepared by following the procedure Example 19. Anhydrous THF (1.0 mL) was added to the reaction vial. The solution was cooled to 0° C. followed by dropwise addition of DIAD (30.3 mg, 0.15 mmol, 1.5 eq). After 5 min, the solution was then allowed to warm to room temperature and stirred for 2 h. A 1:1 Trifluoroethanol/H₂O mixture was added and the mixture was stirred overnight. The crude mixture was concentrated in vacuo and then purified by silica gel flash column chromatography on silica gel eluting with 0-100% EtOAc in hexanes to provide rac-tert-butyl (2-((1S,6S)-6-((tert-butoxycarbonyl)amino)cyclohex-3-en-1-yl)-5-chloro-3-(propa-1,2-dien-1-yl)thieno[3,2-b]pyridin-7-yl)(thiophen-2-ylmethyl)carbamate (27.6 mg, 45% yield). MS m/z 614.2 [M+H]⁺.

### Step 2: rac-2-((1S,6S)-6-Aminocyclohex-3-en-1-yl)-5-chloro-3-(propa-1,2-dien-1-yl)-N-(thiophen-2-ylmethyl)thieno[3,2-b]pyridin-7-amine

Removal of the Boc group following the general procedure described in Step 2 Example 19 provided rac-2-((1S,6S)-6-aminocyclohex-3-en-1-yl)-5-chloro-3-(propa-1,2-dien-1-yl)-N-(thiophen-2-ylmethyl)thieno[3,2-b]pyridin-7-amine (13.6 mg. 73% yield). MS m/z 414.1 [M+H]⁺; ¹H NMR (METHANOL-d₄) δ: 7.30 (dd, J=5.1, 1.2 Hz, 1H), 7.08 (dd, J=3.5, 1.2 Hz, 1H), 6.98 (dd, J=5.1, 3.5 Hz, 1H), 6.75 (t, J=7.1 Hz, 1H), 6.58 (s, 1H), 5.94-5.87 (m, 1H), 5.80-5.74 (m, 1H), 5.28-5.08 (m, 2H), 4.76 (s, 2H), 4.06 (td, J=9.3, 5.7 Hz, 1H), 3.73 (td, J=9.3, 5.4 Hz, 1H), 2.72-2.58 (m, 2H), 2.59-2.43 (m, 1H), 2.34-2.23 (m, 1H). (3 Exchangeable protons not observed).

### Example 27

### Preparation of Compound 303

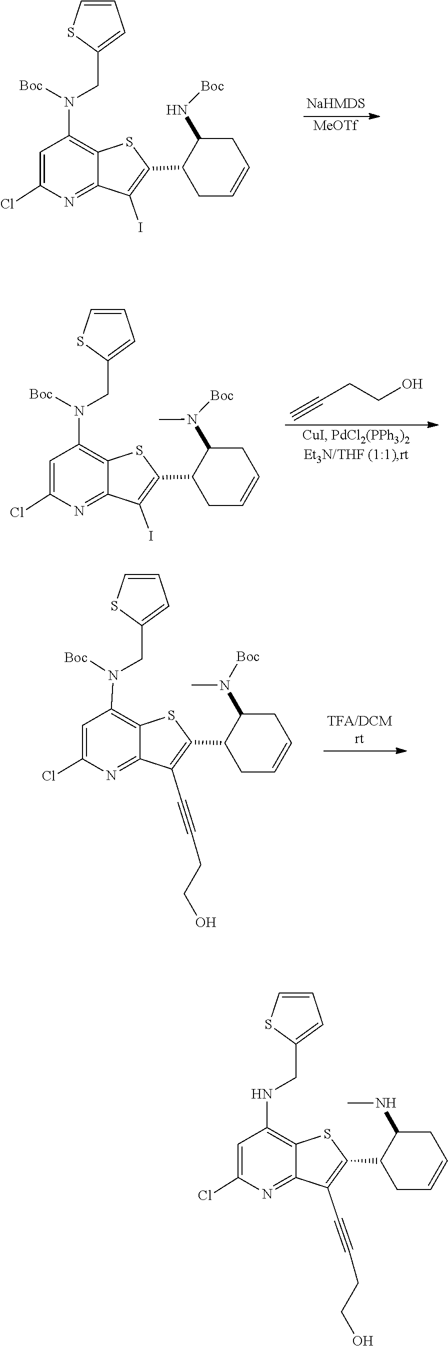

### Step 1: tert-Butyl (rac-(1S,6S)-6-(7-((tert-butoxycarbonyl)(thiophen-2-ylmethyl)amino)-5-chloro-3-iodothieno[3,2-b]pyridin-2-yl)cyclohex-3-en-1-yl)(methyl)carbamate

To a solution of tert-butyl (2-((1S,6S)-6-((tert-butoxycarbonyl)amino)cyclohex-3-en-1-yl)-5-chloro-3-iodothieno[3,2-b]pyridin-7-yl)(thiophen-2-ylmethyl)carbamate (70 mg, 0.1 mmol) (which was prepared according to the procedures in Example 13) in THF (1.0 mL) was added sodium bis(trimethylsilyl)amide (1.0 mol/L) in THF (0.12 mL, 0.12 mmol, 1.2 eq.) dropwise at −78° C., followed by addition of methyl trifluoromethanesulfonate (24.5 mg, 0.15 mmol, 1.5 eq.). The reaction was stirred under −50° C. for 1 hour, then quenched with saturated aqueous NH₄Cl. The organic layer was washed with water, brine and dried over sodium sulfate and evaporated. The residue was purified by flash column chromatography on silica gel eluting with 0-100% EtOAc in hexanes to provide tert-butyl (rac-(1S,6S)-6-(7-((tert-butoxycarbonyl)(thiophen-2-ylmethyl)amino)-5-chloro-3-iodothieno[3,2-b]pyridin-2-yl)cyclohex-3-en-1-yl)(methyl)carbamate (53.7 mg, 75% yield). MS m/z 716.1 [M+H]⁺.

### Step 2: tert-Butyl (rac-(1S,6S)-6-(7-((tert-butoxycarbonyl)(thiophen-2-ylmethyl)amino)-5-chloro-3-(4-hydroxybut-1-yn-1-yl)thieno[3,2-b]pyridin-2-yl)cyclohex-3-en-1-yl)(methyl)carbamate

Installation of the alkyne group following the general procedure described in Step 1 from Example 19 provided tert-butyl (rac-(1S,6S)-6-(7-((tert-butoxycarbonyl)(thiophen-2-ylmethyl)amino)-5-chloro-3-(4-hydroxybut-1-yn-1-yl)thieno[3,2-b]pyridin-2-yl)cyclohex-3-en-1-yl)(methyl)carbamate (42.3 mg. 86% yield). MS m/z 658.2 [M+H]⁺.

### Step 3: 4-(5-Chloro-2-(rac-(1S,6S)-6-(methylamino)cyclohex-3-en-1-yl)-7-((thiophen-2-ylmethyl)amino)thieno[3,2-b]pyridin-3-yl)but-3-yn-1-ol

Removal of the Boc group following the general procedure described in Step 2 from Example 19 provided 4-(5-chloro-2-(rac-(1S,6S)-6-(methylamino)cyclohex-3-en-1-yl)-7-((thiophen-2-ylmethyl)amino)thieno[3,2-b]pyridin-3-yl)but-3-yn-1-ol (17.7 mg. 60% yield). MS m/z 458.1 [M+H]⁺; ¹H NMR (METHANOL-d₄) δ: 7.30 (dd, J=5.1, 1.3 Hz, 1H), 7.09 (dd, J=3.6, 1.3 Hz, 1H), 6.98 (dd, J=5.1, 3.6 Hz, 1H), 6.62 (s, 1H), 5.93 (d, J=10.3 Hz, 1H), 5.81 (d, J=10.3 Hz, 1H), 4.76 (s, 2H), 3.95-3.84 (m, 2H), 3.81 (t, J=6.1 Hz, 2H), 2.77 (t, J=6.1 Hz, 2H), 2.74 (s, 3H), 2.70-2.60 (m, 3H), 2.42-2.30 (m, 1H). (3 Exchangeable protons not observed).

The compounds below were prepared according to Example 27 by substituting the appropriate starting materials, reagents, reaction conditions, and followed by chiral SFC resolution if needed.

| Compound | Spectral Data |
|---|---|
| 288 | MS m/z 472.1 [M + H]⁺; ¹H NMR (METHANOL-d₄) δ: 7.30 (d, J = 5.1 |
| | Hz, 1H), 7.08 (d, J = 3.4 Hz, 1H), 6.98 (dd, J = 5.1, 3.4 Hz, 1H), 6.62 (s, |
| | 1H), 5.93 (d, J = 10.4 Hz, 1H), 5.81 (d, J = 10.4 Hz, 1H), 4.76 (s, 2H), |
| | 3.95-3.78 (m, 2H), 3.74 (t, J = 6.1 Hz, 2H), 2.74 (s, 3H), 2.72-2.60 |
| | (m, 5H), 2.42-2.30 (m, 1H), 1.88 (tt, J = 6.6, 6.6 Hz, 2H). (3 |
| | Exchangeable protons not observed). |
| 264 | MS m/z 474.1 [M + H]⁺; ¹H NMR (METHANOL-d₄) δ: 7.30 (d, J = 5.1 |
| | Hz, 1H), 7.09 (d, J = 3.4 Hz, 1H), 6.98 (dd, J = 5.1, 3.4 Hz, 1H), 6.61 (s, |
| | 1H), 4.76 (s, 2H), 3.76 (t, J = 6.1 Hz, 2H), 3.66-3.52 (m, 1H), 3.51- |
| | 3.40 (m, 1H), 2.70-2.66 (m, 2H), 2.66 (s, 3H), 2.35-2.26 (m, 1H), |
| | 2.15-2.10 (m, 1H), 2.07-1.99 (m, 1H), 1.95-1.82 (m, 4H), 1.60- |
| | 1.45 (m, 3H). (3 Exchangeable protons not observed). |
| 293 | MS m/z 473.1 [M + H]⁺; ¹H NMR (METHANOL-d₄) δ: 7.28 (dd, J = 5.2, |
| | 1.4 Hz, 1H), 7.11-7.07 (m, 1H), 6.95 (dd, J = 5.2, 3.4 Hz, 1H), 5.92 (d, |
| | J = 10.6 Hz, 1H), 5.80 (d, J = 10.6 Hz, 1H), 4.98-4.88 (m, 2H), 3.93- |
| | 3.80 (m, 2H), 3.74 (t, J = 6.1 Hz, 2H), 2.74 (s, 3H), 2.69-2.58 (m, 5H), |
| | 2.40-2.32 (m, 1H), 1.88 (tt, J = 6.6, 6.6 Hz, 2H). (3 Exchangeable |
| | protons not observed). |
| 309 | MS m/z 476.1 [M + H]⁺; ¹H NMR (METHANOL-d₄) δ: 7.30 (d, J = 5.0 |
| | Hz, 1H), 7.11-7.06 (m, 1H), 7.01-6.95 (m, 1H), 6.63 (s, 1H), 5.04 (d, |
| | J = 9.6 Hz, 1H), 4.77 (s, 2H), 4.17-4.10 (m, 1H), 3.82-3.75 (m, 2H), |
| | 3.68 (td, J = 11.4, 3.4 Hz, 1H), 3.48-3.40 (m, 1H), 2.72-2.65 (m, |
| | 5H), 2.47-2.40 (m, 1H), 2.00-1.79 (m, 5H). (3 Exchangeable protons |
| | not observed). |
| 387 | MS m/z [M + H]⁺ 515.9; ¹H NMR (METHANOL-d₄) δ: 8.43 (s, 1H), 7.20 |
| | (dt, J = 5.4, 1.7 Hz, 1H), 6.98 (d, J = 3.7 Hz, 1H), 6.92-6.81 (m, 1H), |
| | 6.48 (d, J = 2.5 Hz, 1H), 5.72 (d, J = 16.6 Hz, 2H), 4.64 (d, J = 2.4 Hz, |
| | 2H), 3.65 (s, 1H), 3.35-3.26 (m, 1H), 2.55-2.44 (m, 2H), 2.41 (s, |
| | 3H), 2.40-2.29 (m, 1H), 2.09 (d, J = 17.9 Hz, 1H). Formic acid salt, (2, |
| | exchangeable protons not observed). |

### Example 28

### Preparation of Compound 259

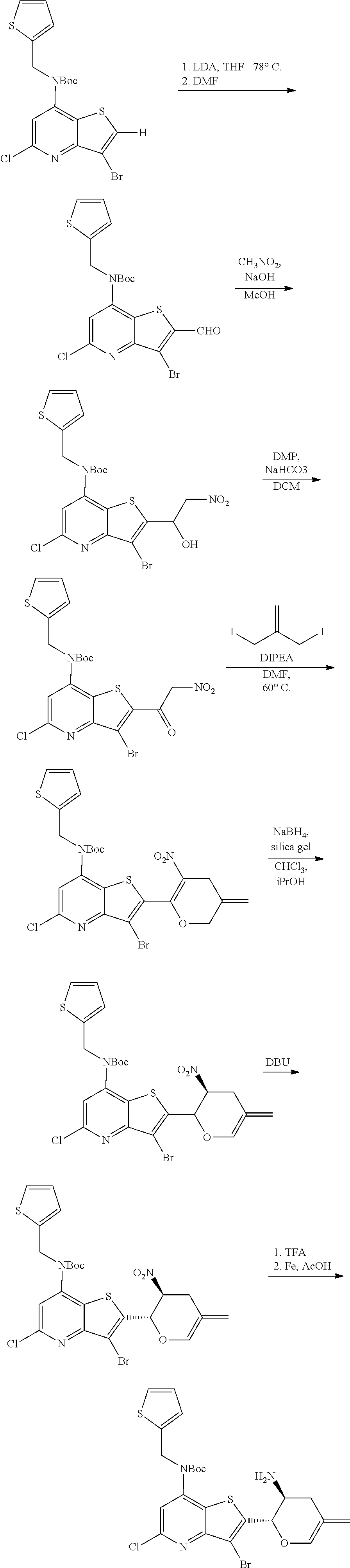

### Step 1: tert-Butyl (3-bromo-5-chloro-2-formylthieno[3,2-b]pyridin-7-yl)(thiophen-2-ylmethyl)carbamate

To a solution of tert-butyl N-(3-bromo-5-chloro-thieno[3,2-b]pyridin-7-yl)-N-(2-thienylmethyl)carbamate (5 g, 11 mmol) (prepared according to the procedure described in WO2020/17430) in THF (50 mL) was added 2M lithium diisopropylamide (6 mL, 1.2 eq.) at −78° C. and stirred for 1 h, followed by addition of DMF (2.4 g) at −78° C. After stirring at −78° C. for an additional 3 h, the reaction mixture was quenched with saturated aqueous NH₄Cl and extracted with EtOAc (2×100 mL). The combined organics were dried over Na₂SO₄, filtered, and concentrated. The crude reside was purified by silica gel column chromatography (DCM/PE 1:3) to afford tert-butyl (3-bromo-5-chloro-2-formylthieno[3,2-b]pyridin-7-yl)(thiophen-2-ylmethyl)carbamate (5.1 g, 96%) as a light yellow solid. MS m/z 488.8 [M+H]⁺. ¹H NMR (400 MHz, CHLOROFORM-d) δ: 10.32 (s, 1H), 7.23 (d, J=7.1 Hz, 2H), 6.87 (dd, J=4.9, 3.6 Hz, 1H), 6.81 (d, J=3.3 Hz, 1H), 5.06 (s, 2H), 1.47 (s, 9H).

### Step 2: tert-Butyl (3-bromo-5-chloro-2-(1-hydroxy-2-nitroethyl)thieno[3,2-b]pyridin-7-yl)(thiophen-2-ylmethyl)carbamate

To a solution of NaOH (1M aqueous, 36 mL) in MeOH (120 mL), cooled to −10° C., was added tert-butyl (3-bromo-5-chloro-2-formylthieno[3,2-b]pyridin-7-yl)(thiophen-2-ylmethyl)carbamate (14.4 g, 30 mmol) followed by addition of nitromethane (2.16 g, 30 mmol, 1.0 eq.) in MeOH (50 mL) dropwise. After stirring at 0° C. for 1 h, the reaction mixture was poured into water (300 mL) and extracted with EtOAc (3×400 mL). The combined organic phase was washed with brine (500 mL), dried over Na₂SO₄, filtered, and concentrated. The crude residue was purified by silica gel column chromatography (EA/PE 0-15% gradient) to afford tert-butyl (3-bromo-5-chloro-2-(1-hydroxy-2-nitroethyl)thieno[3,2-b]pyridin-7-yl)(thiophen-2-ylmethyl)carbamate (13.3 g, 82%) as a yellow oil. ¹H NMR (400 MHz, CHLOROFORM-d) δ: 7.23 (dd, J=5.1, 1.1 Hz, 1H), 6.88 (dd, J=5.1, 3.5 Hz, 1H), 6.80 (d, J=2.6 Hz, 1H), 5.95 (dd, J=6.4, 3.3 Hz, 1H), 5.09-4.96 (m, 2H), 4.78 (dd, J=13.5, 2.6 Hz, 1H), 4.60 (dd, J=13.5, 9.6 Hz, 1H), 4.17 (t, J=8.2 Hz, 1H), 1.48 (s, 9H).

### Step 3: tert-Butyl (3-bromo-5-chloro-2-(2-nitroacetyl)thieno[3,2-b]pyridin-7-yl)(thiophen-2-ylmethyl)carbamate

To a solution of tert-butyl N-[3-bromo-5-chloro-2-(1-hydroxy-2-nitro-ethyl)thieno[3,2-b]pyridin-7-yl]-N-(2-thienylmethyl)carbamate (13.3 g, 24 mmol) in DCM (130 mL), cooled to 0° C., was added Na₂CO₃ (3.98 g, 48 mmol, 2 eq.) and Dess-Martin periodinane (15.1 g, 35.6 mmol, 1.5 eq.). After stirring at 0° C. for 3 h, hexane (400 mL) was added and the solution was stirred at room temperature for 10 min. The solution was then filtered and the filtrate was concentrated in vacuo. The crude residue was purified by silica gel column chromatography (EA/PE, 0-20% gradient) to afford tert-butyl N-[3-bromo-5-chloro-2-(2-nitroacetyl)thieno[3,2-b]pyridin-7-yl]-N-(2-thienylmethyl)carbamate (11 g, 83.%) as a yellow solid. MS m/z 548.0 [M+H]⁺.

### Step 4: tert-Butyl (3-bromo-5-chloro-2-(3-methylene-5-nitro-3,4-dihydro-2H-pyran-6-yl)thieno[3,2-b]pyridin-7-yl)(thiophen-2-ylmethyl)carbamate

To a solution of tert-butyl N-[3-bromo-5-chloro-2-(2-nitroacetyl)thieno[3,2-b]pyridin-7-yl]-N-(2-thienylmethyl)carbamate (11 g, 20.12 mmol) in DMF (110 mL) was added 3-iodo-2-(iodomethyl)prop-1-ene (11.55 g, 37.51 mmol, 1.9 eq.) and DIPEA (6.49 g, 50.2 mmol, 2.5 eq.). After stirring at 60° C. for 2 h, the reaction mixture was cooled to room temperature and diluted with water (100 mL). The aqueous phase was extracted with EtOAc (3×100 mL) and the combined organics were washed with brine, dried over Na₂SO₄, filtered, and concentrated in vacuo. The crude residue was purified by silica gel column chromatography (EA/PE 0-20% gradient) to afford tert-butyl N-[3-bromo-5-chloro-2-(5-methylene-3-nitro-4H-pyran-2-yl)thieno[3,2-b]pyridin-7-yl]-N-(2-thienylmethyl)carbamate (5.5 g, 46%) as a colorless oil. MS m/z 599.7 [M+H]⁺.

### Step 5: tert-Butyl (3-bromo-5-chloro-2-(5-methylene-3-nitrotetrahydro-2H-pyran-2-yl)thieno[3,2-b]pyridin-7-yl)(thiophen-2-ylmethyl)carbamate

To a solution of tert-butyl N-[3-bromo-5-chloro-2-(5-methylene-3-nitro-4H-pyran-2-yl)thieno[3,2-b]pyridin-7-yl]-N-(2-thienylmethyl)carbamate (5.5 g, 9.2 mmol) in chloroform (165 mL) and 2-propanol (33 mL) was added silica gel (14.6 g) and NaBH₄ (1.3 g, 34 mmol, 3.7 eq.) at 25° C. After stirring at room temperature for 2 h, the reaction mixture was quenched with aqueous NH₄Cl (1M, 100 mL), diluted with water, and extracted with EtOAc (3×100 mL). The combined organics were dried over Na₂SO₄, filtered, and concentrated in vacuo. The crude residue was purified by silica gel column chromatography (EA/PE 0-20% gradient) to afford tert-butyl (3-bromo-5-chloro-2-(5-methylene-3-nitrotetrahydro-2H-pyran-2-yl)thieno[3,2-b]pyridin-7-yl)(thiophen-2-ylmethyl)carbamate (2.7 g, 2.0 mmol, 67%) as a mixture of diastereomers. MS m/z 602.0 [M+H]⁺.

### Step 6: tert-Butyl (3-bromo-5-chloro-2-((2S,3S)-5-methylene-3-nitrotetrahydro-2H-pyran-2-yl)thieno[3,2-b]pyridin-7-yl)(thiophen-2-ylmethyl)carbamate

To a solution of tert-butyl (3-bromo-5-chloro-2-(5-methylene-3-nitrotetrahydro-2H-pyran-2-yl)thieno[3,2-b]pyridin-7-yl)(thiophen-2-ylmethyl)carbamate (680 mg, 1.1 mmol) in THF (20 mL) was added dropwise 2,3,4,6,7,8,9,10-octahydropyrimido[1,2-a]azepine (86 mg, 0.56 mmol, 0.5 eq.) at 0° C. under N₂. The mixture was stirred for 2 h at 0° C. The reaction was then quenched with water (50 ml), and extracted with EtOAc (50 ml×2). The combined organic layers were washed with brine (100 ml), dried over Na₂SO₄, filtered and concentrated in vacuo. The crude residue was purified by silica gel column chromatography (EA/PE 0-20% gradient) to afford tert-butyl (3-bromo-5-chloro-2-((2S,3S)-5-methylene-3-nitrotetrahydro-2H-pyran-2-yl)thieno[3,2-b]pyridin-7-yl)(thiophen-2-ylmethyl)carbamate (260 mg, 38%) as a white solid. MS m/z 602.0 [M+H]⁺.

### Step 7: rac-2-((2S,3S)-3-Amino-5-methylenetetrahydro-2H-pyran-2-yl)-3-bromo-5-chloro-N-(thiophen-2-ylmethyl)thieno[3,2-b]pyridin-7-amine

To a solution of afford tert-butyl (3-bromo-5-chloro-2-((2S,3S)-5-methylene-3-nitrotetrahydro-2H-pyran-2-yl)thieno[3,2-b]pyridin-7-yl)(thiophen-2-ylmethyl)carbamate (1 g, 2 mmol) in EtOH (15 mL) and AcOH (5 mL) was added iron (1.55 g, 28 mmol, 2.9 eq.) at 25° C. After stirring for 12 h at 65° C., the reaction mixture was cooled to room temperature and filtered, rinsing with DCM (50 mL×2). The filtrate was concentrated and the crude residue was purified by silica gel column chromatography (MeOH/DCM 0-10% gradient) to give rac-2-((2S,3S)-3-amino-5-methylenetetrahydro-2H-pyran-2-yl)-3-bromo-5-chloro-N-(thiophen-2-ylmethyl)thieno[3,2-b]pyridin-7-amine (870 mg, 92%) as a yellow solid. MS m/z 471.8 [M+H]⁺. ¹H NMR (400 MHz, METHANOL-d₄) δ: 7.29 (dd, J=5.1, 1.2 Hz, 1H), 7.08 (dd, J=3.5, 1.0 Hz, 1H), 6.97 (dd, J=5.1, 3.5 Hz, 1H), 6.63 (s, 1H), 5.14 (t, J=12.2 Hz, 3H), 4.76 (s, 2H), 4.41 (d, J=12.6 Hz, 1H), 4.26 (d, J=12.7 Hz, 1H), 3.46 (ddd, J=11.7, 9.7, 4.7 Hz, 1H), 2.95 (dd, J=13.5, 4.0 Hz, 1H), 2.66 (d, J=12.3 Hz, 1H) (3 exchangeable protons are not seen).

### Example 29

### Preparation of Compound 284

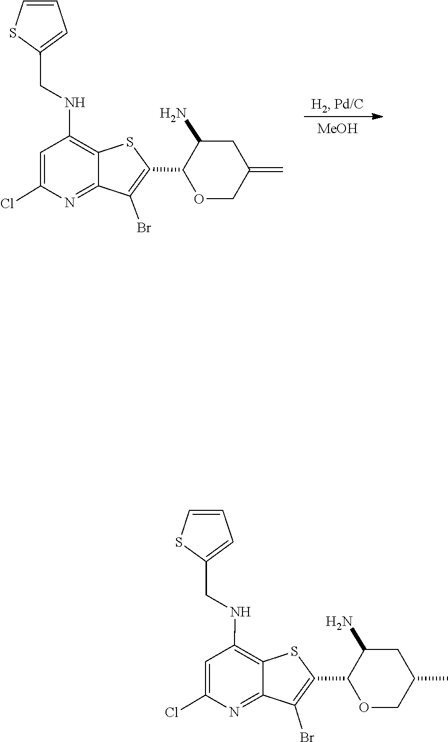 [SMILES: "MeOH" ; C=C1CO[C@H](c2sc3c(NCc4cccs4)cc(Cl)nc3c2Br)[C@@H](N)C1 ; "H2, Pd/C" ; C[C@@H]1CO[C@H](c2sc3c(NCc4cccs4)cc(Cl)nc3c2Br)[C@@H](N)C1]

### Step 1: rac-2-((2S,3S,5S)-3-Amino-5-methyltetrahydro-2H-pyran-2-yl)-3-bromo-5-chloro-N-(thiophen-2-ylmethyl)thieno[3,2-b]pyridin-7-amine

To a 1.0 mL MeOH solution of rac-2-((2S,3S)-3-amino-5-methylenetetrahydro-2H-pyran-2-yl)-3-bromo-5-chloro-N-(thiophen-2-ylmethyl)thieno[3,2-b]pyridin-7-amine (23.6 mg, 0.05 mmol) which was provided by following the procedure in Example 28, was added Pd/C (10 wt. % loading, 10.6 mg, 0.01 mmol, 0.2 eq.). The reaction mixture was sparged by hydrogen gas for 5 min using a hydrogen balloon. The reaction was then allowed to stir at room temperature under the hydrogen balloon for 2 h. The catalyst was filtered off and washed with MeOH. The mixture was concentrated and purified by flash column chromatography on silica gel eluting with 0-40% MeOH in DCM to provide rac-2-((2S,3S,5S)-3-amino-5-methyltetrahydro-2H-pyran-2-yl)-3-bromo-5-chloro-N-(thiophen-2-ylmethyl)thieno[3,2-b]pyridin-7-amine (20.8 mg, 88% yield). MS m/z 472.0 [M+H]⁺; ¹H NMR (METHANOL-d₄) δ: 7.30 (dd, J=5.1, 1.3 Hz, 1H), 7.12-7.06 (m, 1H), 6.98 (dd, J=5.1, 3.6 Hz, 1H), 6.64 (s, 1H), 4.98 (d, J=9.4 Hz, 1H), 4.77 (s, 2H), 3.88-3.83 (m, 2H), 3.73-3.59 (m, 1H), 2.24-2.14 (m, 1H), 2.14-2.00 (m, 2H), 1.28 (d, J=7.1 Hz, 3H). (3 Exchangeable protons not observed).

### BIOLOGICAL EXAMPLES

The following in vitro biological examples demonstrate the usefulness of the compounds of the present description for treating SCA3.

To describe in more detail and assist in understanding the present description, the following non-limiting biological examples are offered to more fully illustrate the scope of the description and are not to be construed as specifically limiting the scope thereof. Such variations of the present description that may be now known or later developed, which would be within the purview of one skilled in the art to ascertain, are considered to fall within the scope of the present description and as hereinafter claimed.

### Example 1

Endogenous Total ATXN3 (tATXN3) Protein Assay

Meso Scale Discovery (MSD) 96-well or 384-well plates were coated overnight at 4° C. with Ataxin 3 mouse monoclonal antibody (Invitrogen, MA3-082) at a concentration of 0.5 μg/mL in PBS (30 μL per well). Plates were then washed three times with 300 μL wash buffer (0.05% TWEEN®-20, polyethylene glycol sorbitan monolaurate, in PBS) and blocked (Meso Scale Diagnostics, R93BA-1; 5% BSA in PBS) for 2 h at RT with rotational shaking and then washed three times with wash buffer.

Test compounds were serially diluted 3.16-fold in 100% DMSO to generate a 7-point concentration curve. Aliquots of 0.5 μL of diluted compounds were transferred to a 96-well flat bottom plate by a liquid handler. An aliquot of 0.5 μL DMSO was also transferred to separate wells and used as controls. Duplicate samples were set up for each compound concentration and for the DMSO control.

Cells were thawed and incubated in cell culture media (DMEM, 10% FBS, and 1% antibiotic cocktail) for 72 h. Cells were trypsinized, counted, and re-suspended to a concentration of 100,000 cells/mL in cell culture media. A 100 μL aliquot of the cell suspensions were plated at 10,000 cells per well in the compound containing 96 well microtiter plate and incubated for in a cell culture incubator (37° C., 5% CO₂, 100% relative humidity). After 48 h, the medium was removed and 50-100 μL of lysis buffer (Meso Scale Diagnostics, R60TX-2) containing 1× halt protease inhibitor cocktail of (Thermo Scientific, Halt™ Protease Inhibitor Cocktail, 78430) per well was added to cells to provide a “cell lysate”. The plate was placed on a shaker at 4° C. for 30 minutes, then stored at −80° C.

Cell lysate samples (25 μL) were transferred to the antibody-coated MSD plate and incubated overnight at 4° C. After removal of the lysates, the plate was washed three times with wash buffer, and 25 μL of Ataxin 3 recombinant rabbit monoclonal antibody (Invitrogen, #702788) secondary antibody (diluted to 0.25 μg/mL in 0.05% TWEEN®-20 in blocking buffer) was added to each well and incubated with shaking for 1-2 h at room temperature. Following incubation with the secondary antibody, the wells were rinsed with wash buffer and then 25 μL of Anti Rabbit Antibody Goat SULFO-TAG Labeled (Meso Scale Diagnostics, R32AB-1) detecting antibody (diluted to 0.25 g/mL in 0.05% TWEEN®-20 in blocking buffer) was added to each well and incubated with shaking for 1 h at room temperature. After rinsing three times with wash buffer, 150 μL of Read Buffer T with surfactant (tris-based buffer containing tripropylamine, Meso Scale Diagnostics, R92TC-1) were added to each empty well, and the plate was imaged on a SI 6000 imager (MSD) according to manufacturers' instructions provided for 96- or 384-well plates. The resulting average IC₅₀ values (μM) for the representative compounds tested are shown in Table 1.

An average IC₅₀>1 μM is indicated by one star (*), between >0.3 μM and ≤1 μM is indicated by two stars (**), between >0.03 μM and ≤0.3 μM is indicated by three stars (***), and ≤0.03 μM is indicated by four stars (****).

TABLE 1

| Cpd | IC₅₀ |
|---|---|
| 1 | **** |
| 2 | * |
| 3 | * |
| 4 | *** |
| 5 | * |
| 6 | *** |
| 7 | ** |
| 8 | **** |
| 9 | * |
| 10 | ** |
| 11 | ** |
| 12 | * |
| 13 | *** |
| 14 | * |
| 15 | ** |
| 16 | * |
| 17 | * |
| 18 | **** |
| 19 | *** |
| 20 | *** |
| 21 | *** |
| 22 | ** |
| 23 | * |
| 24 | *** |
| 25 | *** |
| 26 | ** |
| 27 | *** |
| 28 | * |
| 29 | *** |
| 30 | * |
| 31 | * |
| 32 | *** |
| 33 | *** |
| 34 | **** |
| 35 | ** |
| 36 | * |
| 37 | ** |
| 38 | *** |
| 39 | * |
| 40 | *** |
| 41 | *** |
| 42 | ** |
| 43 | * |
| 44 | * |
| 45 | **** |
| 46 | * |
| 47 | * |
| 48 | *** |
| 49 | * |
| 50 | ** |
| 51 | * |
| 52 | * |
| 53 | ** |
| 54 | *** |
| 55 | *** |
| 56 | ** |
| 57 | **** |
| 58 | * |
| 59 | * |
| 60 | **** |
| 61 | * |
| 62 | *** |
| 63 | *** |
| 64 | * |
| 65 | **** |
| 66 | * |
| 67 | ** |
| 68 | **** |
| 69 | *** |
| 70 | * |
| 71 | **** |
| 72 | * |
| 73 | ** |
| 74 | * |
| 75 | *** |
| 76 | * |
| 77 | * |
| 78 | * |
| 79 | *** |
| 80 | * |
| 81 | *** |
| 82 | ** |
| 83 | * |
| 84 | * |
| 85 | * |
| 86 | * |
| 87 | *** |
| 88 | * |
| 89 | * |
| 90 | ** |
| 91 | * |
| 92 | * |
| 93 | * |
| 94 | ** |
| 95 | * |
| 96 | *** |
| 97 | * |
| 98 | * |
| 99 | *** |
| 100 | * |
| 101 | * |
| 102 | * |
| 103 | * |
| 104 | * |
| 105 | * |
| 106 | * |
| 107 | ** |
| 108 | **** |
| 109 | *** |
| 110 | *** |
| 111 | * |
| 112 | * |
| 113 | * |
| 114 | * |
| 115 | **** |
| 116 | **** |
| 117 | **** |
| 118 | ** |
| 119 | * |
| 120 | *** |
| 121 | * |
| 122 | * |
| 123 | * |
| 124 | * |
| 125 | * |
| 126 | *** |
| 127 | *** |
| 128 | * |
| 129 | ** |
| 130 | *** |
| 131 | * |
| 132 | ** |
| 133 | *** |
| 134 | * |
| 135 | * |
| 136 | **** |
| 137 | * |
| 138 | *** |
| 139 | *** |
| 140 | ** |
| 141 | * |
| 142 | * |
| 143 | **** |
| 144 | **** |
| 145 | * |
| 146 | * |
| 147 | * |
| 148 | * |
| 149 | *** |
| 150 | *** |
| 151 | *** |
| 152 | *** |
| 153 | **** |
| 154 | * |
| 155 | * |
| 156 | * |
| 157 | * |
| 158 | * |
| 159 | * |
| 160 | * |
| 161 | * |
| 162 | **** |
| 163 | *** |
| 164 | * |
| 165 | **** |
| 166 | *** |
| 167 | ** |
| 168 | **** |
| 169 | **** |
| 170 | **** |
| 171 | **** |
| 172 | **** |
| 173 | **** |
| 174 | **** |
| 175 | **** |
| 176 | **** |
| 177 | **** |
| 178 | **** |
| 179 | **** |
| 180 | **** |
| 181 | **** |
| 182 | **** |
| 183 | **** |
| 184 | **** |
| 185 | **** |
| 186 | **** |
| 187 | **** |
| 188 | **** |
| 189 | **** |
| 190 | **** |
| 191 | **** |
| 192 | **** |
| 193 | **** |
| 194 | **** |
| 195 | **** |
| 196 | **** |
| 197 | **** |
| 198 | **** |
| 199 | **** |
| 200 | **** |
| 201 | **** |
| 202 | **** |
| 203 | **** |
| 204 | **** |
| 205 | **** |
| 206 | **** |
| 207 | **** |
| 208 | **** |
| 209 | **** |
| 210 | **** |
| 211 | **** |
| 212 | **** |
| 213 | **** |
| 214 | **** |
| 215 | **** |
| 216 | **** |
| 217 | **** |
| 218 | **** |
| 219 | **** |
| 220 | **** |
| 221 | **** |
| 222 | **** |
| 223 | **** |
| 224 | **** |
| 225 | **** |
| 226 | **** |
| 227 | **** |
| 228 | **** |
| 229 | **** |
| 230 | **** |
| 231 | **** |
| 232 | **** |
| 233 | **** |
| 234 | **** |
| 235 | **** |
| 236 | **** |
| 237 | **** |
| 238 | **** |
| 239 | **** |
| 240 | **** |
| 241 | **** |
| 242 | **** |
| 243 | **** |
| 244 | **** |
| 245 | **** |
| 246 | **** |
| 247 | *** |
| 248 | *** |
| 249 | *** |
| 250 | *** |
| 251 | *** |
| 252 | *** |
| 253 | *** |
| 254 | *** |
| 255 | *** |
| 256 | *** |
| 257 | *** |
| 258 | *** |
| 259 | *** |
| 260 | *** |
| 261 | *** |
| 262 | *** |
| 263 | *** |
| 264 | *** |
| 265 | *** |
| 266 | *** |
| 267 | *** |
| 268 | *** |
| 269 | *** |
| 270 | *** |
| 271 | *** |
| 272 | *** |
| 273 | *** |
| 274 | *** |
| 275 | *** |
| 276 | *** |
| 277 | *** |
| 278 | *** |
| 279 | *** |
| 280 | *** |
| 281 | *** |
| 282 | *** |
| 283 | *** |
| 284 | *** |
| 285 | *** |
| 286 | *** |
| 287 | *** |
| 288 | *** |
| 289 | *** |
| 290 | *** |
| 291 | *** |
| 292 | *** |
| 293 | *** |
| 294 | *** |
| 295 | *** |
| 296 | *** |
| 297 | *** |
| 298 | *** |
| 299 | *** |
| 300 | *** |
| 301 | *** |
| 302 | *** |
| 303 | *** |
| 304 | *** |
| 305 | *** |
| 306 | *** |
| 307 | *** |
| 308 | *** |
| 309 | *** |
| 310 | *** |
| 311 | *** |
| 312 | *** |
| 313 | *** |
| 314 | *** |
| 315 | *** |
| 316 | *** |
| 317 | *** |
| 318 | *** |
| 319 | *** |
| 320 | *** |
| 321 | *** |
| 322 | *** |
| 323 | *** |
| 324 | *** |
| 325 | *** |
| 326 | *** |
| 327 | *** |
| 328 | *** |
| 329 | *** |
| 330 | *** |
| 331 | *** |
| 332 | *** |
| 333 | *** |
| 334 | *** |
| 335 | *** |
| 336 | *** |
| 337 | *** |
| 338 | *** |
| 339 | *** |
| 340 | *** |
| 341 | *** |
| 342 | *** |
| 343 | *** |
| 344 | *** |
| 345 | *** |
| 346 | *** |
| 347 | *** |
| 348 | *** |
| 349 | *** |
| 350 | *** |
| 351 | *** |
| 352 | *** |
| 353 | *** |
| 354 | *** |
| 355 | *** |
| 356 | *** |
| 357 | *** |
| 358 | *** |
| 359 | *** |
| 360 | *** |
| 361 | *** |
| 362 | *** |
| 363 | *** |
| 364 | *** |
| 365 | *** |
| 366 | *** |
| 367 | *** |
| 368 | *** |
| 369 | *** |
| 370 | *** |
| 371 | *** |
| 372 | *** |
| 373 | *** |
| 374 | *** |
| 375 | *** |
| 376 | *** |
| 377 | *** |
| 378 | *** |
| 379 | *** |
| 380 | *** |
| 381 | *** |
| 382 | *** |
| 383 | *** |
| 384 | *** |
| 385 | *** |
| 386 | *** |
| 387 | *** |
| 388 | *** |
| 389 | ** |
| 390 | ** |
| 391 | ** |
| 392 | ** |
| 393 | ** |
| 394 | ** |
| 395 | ** |
| 396 | ** |
| 397 | ** |
| 398 | ** |
| 399 | ** |
| 400 | ** |
| 401 | * |
| 402 | **** |
| 403 | ** |
| 404 | *** |
| 405 | **** |
| 406 | *** |
| 407 | *** |
| 408 | *** |
| 409 | *** |
| 410 | *** |
| 411 | *** |
| 412 | ** |
| 413 | *** |
| 414 | ** |
| 415 | ** |
| 416 | *** |
| 417 | **** |
| 418 | *** |
| 419 | *** |
| 420 | *** |
| 421 | **** |
| 422 | *** |
| 423 | *** |
| 424 | *** |
| 425 | * |
| 426 | ** |
| 427 | *** |
| 428 | ** |
| 429 | **** |
| 430 | *** |
| 431 | **** |
| 432 | *** |
| 433 | *** |
| 434 | *** |
| 435 | *** |
| 436 | * |
| 437 | **** |
| 438 | ** |
| 439 | ** |
| 440 | * |
| 441 | *** |
| 442 | *** |
| 443 | **** |
| 444 | * |
| 445 | ** |
| 446 | **** |
| 447 | ** |
| 448 | * |
| 449 | *** |
| 450 | ** |
| 451 | *** |
| 452 | *** |
| 453 | ** |
| 454 | ** |
| 455 | ** |

### Example 2

RT-qPCR Assay to Quantify Exon 4 Skipping in ATXN3 pre-mRNA in Cells

Test compounds were serially diluted 3.16-fold in 100% DMSO to generate a 7-point concentration curve. Aliquots of 0.5 μL of diluted compounds were transferred to a 96-well flat bottom plate by a liquid handler. An aliquot of 0.5 μL DMSO was also transferred to separate wells and used as controls. Duplicate samples were set up for each compound concentration and for the DMSO control.

Cells were thawed and incubated in cell culture media (DMEM, 10% FBS, and 1% antibiotic cocktail) for 72 h. Cells were trypsinized, counted, and re-suspended to a concentration of 100,000 cells/mL in cell culture media. A 100 μL aliquot of the cell suspensions were plated at 10,000 cells per well in the compound containing 96 well microtiter plate and incubated for in a cell culture incubator (37° C., 5% CO₂, 100% relative humidity).

After 24 h, media was aspirated from the cells and 50 μL of the RCL2 lysis buffer (10 mM Tris-HCL pH 7.4, 150 mM NaCl, 0.33% IGEPAL® CA-630) was added to each well and incubated at RT for 1 min. Chilled nuclease free water (50 μL per well) was added and the plates were immediately transferred on ice. After 1 min on ice, plates were frozen at −80° C. overnight.

### Preparation of RT-qPCR Reaction Mixture:

| | Volume | |
|---|---|---|
| Reagent | (μL) | Supplier and Catalogue No. |
| RT-PCR buffer (2X) | 5.0 | Thermo Fisher, 4387391 |
| RT-PCR enzyme mixture (25X) | 0.4 | Thermo Fisher, 4387391 |
| Target Primer/Probe (60X) | 0.16 | Thermo Fisher, 4331182 |
| In house GAPDH assay (20X) | 0.5 | |
| H₂O | 1.94 | |

Abbreviations:
GAPDH, glyceraldehyde 3-phosphate dehydrogenase
Target: TaqMan assay ID Hs00245261_m1
In house GAPDH assay:
Forward primer - 5′ caacggatttggtcgtattgg 3′
Reverse primer - 5′ tgatggcaacaatatccactttacc 3′
Probe (VIC-TAMRA) - 5′ cgcctggtcaccagggctgct 3′

An aliquot of 2 μL/well of the cell lysates was transferred using the liquid handler to the Armadillo 384-Well PCR plate containing 8 μL/well of the RT-qPCR reaction mixture that was prepared as detailed above. The plates were then sealed with MicroAmp™ Optical Adhesive Film followed by spinning down for 1 min and placed in the CFX384 thermocycler (BioRad).

The RT-qPCR was carried out at the following temperatures for the indicated time:
Step 1: 48° C. (30 min)
Step 2: 95° C. (10 min)
Step 3: 95° C. (15 sec)
Step 4: 60° C. (1 min);
   then, repeated Steps 3 and 4 for a total of 40 cycles.

The percent exon 4 skipping was calculated for each dose of compound treatment using Equations 1 and 2.

$$Realtive gene expression = \frac{{2}^{- Ct ( target )}}{{1.9}^{- Ct ( GAPDH )}} \qquad Equation 1$$

$$Percent exon 4 skipping ( \% ) = 1 - [ \frac{Realtive gene expression , Compound}{Realtive gene expression , DMSO} ] \times 100 \qquad Equation 2$$

Data were fit to a dose response curve and the IC₅₀ was interpolated using XLfit® statistical and curve fitting package. The resulting IC₅₀ values (μM) for the representative compounds tested are shown in Table 2.

An average IC₅₀>1 μM is indicated by one star (*), between >0.3 μM and ≤1 μM is indicated by two stars (**), between >0.03 μM and ≤0.3 μM is indicated by three stars (***), and ≤0.03 μM is indicated by four stars (****).

TABLE 2

| Cpd | IC₅₀ |
|---|---|
| 1 | **** |
| 2 | * |
| 3 | * |
| 4 | *** |
| 5 | * |
| 6 | *** |
| 7 | ** |
| 8 | **** |
| 9 | ** |
| 10 | ** |
| 11 | *** |
| 12 | * |
| 13 | *** |
| 14 | * |
| 15 | ** |
| 16 | * |
| 17 | * |
| 18 | **** |
| 19 | *** |
| 20 | *** |
| 21 | *** |
| 22 | ** |
| 23 | * |
| 24 | *** |
| 25 | *** |
| 26 | ** |
| 27 | **** |
| 28 | * |
| 29 | *** |
| 30 | * |
| 31 | * |
| 32 | *** |
| 33 | *** |
| 34 | **** |
| 35 | ** |
| 36 | * |
| 37 | *** |
| 38 | *** |
| 39 | * |
| 40 | *** |
| 41 | *** |
| 42 | ** |
| 43 | * |
| 44 | * |
| 45 | **** |
| 46 | * |
| 47 | ** |
| 48 | *** |
| 49 | * |
| 50 | *** |
| 51 | * |
| 52 | * |
| 53 | ** |
| 54 | *** |
| 55 | *** |
| 56 | *** |
| 57 | **** |
| 58 | * |
| 59 | ** |
| 60 | **** |
| 61 | * |
| 62 | **** |
| 63 | *** |
| 64 | * |
| 65 | **** |
| 66 | * |
| 67 | ** |
| 68 | **** |
| 69 | **** |
| 70 | * |
| 71 | **** |
| 72 | * |
| 73 | ** |
| 74 | * |
| 75 | *** |
| 76 | * |
| 77 | ** |
| 78 | * |
| 79 | *** |
| 80 | * |
| 81 | *** |
| 82 | ** |
| 83 | * |
| 84 | * |
| 85 | * |
| 86 | * |
| 87 | *** |
| 88 | * |
| 89 | * |
| 90 | ** |
| 91 | * |
| 92 | * |
| 93 | * |
| 94 | *** |
| 95 | * |
| 96 | *** |
| 97 | * |
| 98 | * |
| 99 | *** |
| 100 | * |
| 101 | * |
| 102 | * |
| 103 | * |
| 104 | * |
| 105 | * |
| 106 | * |
| 107 | ** |
| 108 | **** |
| 109 | *** |
| 110 | **** |
| 111 | ** |
| 112 | * |
| 113 | * |
| 114 | * |
| 115 | **** |
| 116 | **** |
| 117 | **** |
| 118 | ** |
| 119 | * |
| 120 | *** |
| 121 | * |
| 122 | * |
| 123 | * |
| 124 | * |
| 125 | * |
| 126 | *** |
| 127 | *** |
| 128 | * |
| 129 | *** |
| 130 | *** |
| 131 | * |
| 132 | * |
| 133 | **** |
| 134 | * |
| 135 | * |
| 136 | **** |
| 137 | * |
| 138 | ** |
| 139 | **** |
| 140 | ** |
| 141 | * |
| 142 | * |
| 143 | **** |
| 144 | **** |
| 145 | *** |
| 146 | * |
| 147 | * |
| 148 | * |
| 149 | *** |
| 150 | *** |
| 151 | *** |
| 152 | *** |
| 153 | **** |
| 154 | * |
| 155 | ** |
| 156 | * |
| 157 | * |
| 158 | * |
| 159 | * |
| 160 | * |
| 161 | * |
| 162 | **** |
| 163 | **** |
| 164 | ** |
| 165 | **** |
| 166 | *** |
| 167 | *** |
| 168 | **** |
| 169 | **** |
| 170 | ***** |
| 171 | **** |
| 172 | **** |
| 173 | **** |
| 174 | **** |
| 175 | **** |
| 176 | **** |
| 177 | **** |
| 178 | **** |
| 179 | **** |
| 180 | **** |
| 181 | **** |
| 182 | **** |
| 183 | **** |
| 184 | **** |
| 185 | **** |
| 186 | **** |
| 187 | **** |
| 188 | **** |
| 189 | **** |
| 190 | **** |
| 191 | **** |
| 192 | **** |
| 193 | **** |
| 194 | **** |
| 195 | **** |
| 196 | **** |
| 197 | **** |
| 198 | **** |
| 199 | **** |
| 200 | **** |
| 201 | **** |
| 202 | **** |
| 203 | **** |
| 204 | **** |
| 205 | **** |
| 206 | **** |
| 207 | **** |
| 208 | **** |
| 209 | **** |
| 210 | **** |
| 211 | **** |
| 212 | **** |
| 213 | **** |
| 214 | **** |
| 215 | **** |
| 216 | **** |
| 217 | **** |
| 218 | **** |
| 219 | **** |
| 220 | **** |
| 221 | **** |
| 222 | **** |
| 223 | **** |
| 224 | **** |
| 225 | **** |
| 226 | **** |
| 227 | **** |
| 228 | **** |
| 229 | **** |
| 230 | **** |
| 231 | **** |
| 232 | **** |
| 233 | **** |
| 234 | **** |
| 235 | **** |
| 236 | **** |
| 237 | **** |
| 238 | *** |
| 239 | **** |
| 240 | **** |
| 241 | **** |
| 242 | **** |
| 243 | **** |
| 244 | **** |
| 245 | **** |
| 246 | *** |
| 247 | *** |
| 248 | *** |
| 249 | **** |
| 250 | *** |
| 251 | **** |
| 252 | **** |
| 253 | **** |
| 254 | *** |
| 255 | **** |
| 256 | **** |
| 257 | **** |
| 258 | **** |
| 259 | *** |
| 260 | **** |
| 261 | *** |
| 262 | *** |
| 263 | *** |
| 264 | **** |
| 265 | **** |
| 266 | *** |
| 267 | *** |
| 268 | *** |
| 269 | *** |
| 270 | *** |
| 271 | *** |
| 272 | *** |
| 273 | **** |
| 274 | *** |
| 275 | *** |
| 276 | *** |
| 277 | **** |
| 278 | *** |
| 279 | *** |
| 280 | *** |
| 281 | *** |
| 282 | *** |
| 283 | *** |
| 284 | *** |
| 285 | *** |
| 286 | *** |
| 287 | *** |
| 288 | *** |
| 289 | *** |
| 290 | *** |
| 291 | *** |
| 292 | *** |
| 293 | *** |
| 294 | *** |
| 295 | *** |
| 296 | *** |
| 297 | *** |
| 298 | *** |
| 299 | *** |
| 300 | *** |
| 301 | *** |
| 302 | *** |
| 303 | *** |
| 304 | *** |
| 305 | *** |
| 306 | *** |
| 307 | *** |
| 308 | *** |
| 309 | *** |
| 310 | *** |
| 311 | *** |
| 312 | *** |
| 313 | *** |
| 314 | *** |
| 315 | *** |
| 316 | *** |
| 317 | *** |
| 318 | *** |
| 319 | *** |
| 320 | *** |
| 321 | *** |
| 322 | *** |
| 323 | *** |
| 324 | *** |
| 325 | *** |
| 326 | *** |
| 327 | *** |
| 328 | *** |
| 329 | *** |
| 330 | *** |
| 331 | *** |
| 332 | *** |
| 333 | *** |
| 334 | *** |
| 335 | *** |
| 336 | *** |
| 337 | *** |
| 338 | *** |
| 339 | *** |
| 340 | *** |
| 341 | *** |
| 342 | *** |
| 343 | *** |
| 344 | *** |
| 345 | *** |
| 346 | *** |
| 347 | *** |
| 348 | *** |
| 349 | *** |
| 350 | *** |
| 351 | *** |
| 352 | *** |
| 353 | *** |
| 354 | *** |
| 355 | *** |
| 356 | *** |
| 357 | *** |
| 358 | *** |
| 359 | *** |
| 360 | *** |
| 361 | *** |
| 362 | *** |
| 363 | *** |
| 364 | *** |
| 365 | *** |
| 366 | *** |
| 367 | *** |
| 368 | *** |
| 369 | *** |
| 370 | *** |
| 371 | *** |
| 372 | *** |
| 373 | *** |
| 374 | *** |
| 375 | *** |
| 376 | *** |
| 377 | *** |
| 378 | *** |
| 379 | *** |
| 380 | *** |
| 381 | *** |
| 382 | *** |
| 383 | *** |
| 384 | *** |
| 385 | *** |
| 386 | *** |
| 387 | ** |
| 388 | *** |
| 389 | ** |
| 390 | *** |
| 391 | ** |
| 392 | *** |
| 393 | **** |
| 394 | ** |
| 395 | *** |
| 396 | *** |
| 397 | ** |
| 398 | *** |
| 399 | ** |
| 400 | ** |
| 401 | ** |
| 402 | **** |
| 403 | *** |
| 404 | *** |
| 405 | **** |
| 406 | **** |
| 407 | *** |
| 408 | *** |
| 409 | *** |
| 410 | *** |
| 411 | *** |
| 412 | ** |
| 413 | *** |
| 414 | *** |
| 415 | ** |
| 416 | *** |
| 417 | **** |
| 418 | *** |
| 419 | *** |
| 420 | *** |
| 421 | **** |
| 422 | *** |
| 423 | **** |
| 424 | **** |
| 425 | ** |
| 426 | *** |
| 427 | **** |
| 428 | ** |
| 429 | **** |
| 430 | *** |
| 431 | **** |
| 432 | *** |
| 433 | *** |
| 434 | *** |
| 435 | **** |
| 436 | ** |
| 437 | **** |
| 438 | *** |
| 439 | ** |
| 440 | ** |
| 441 | *** |
| 442 | *** |
| 443 | **** |
| 444 | ** |
| 445 | ** |
| 446 | **** |
| 447 | ** |
| 448 | *** |
| 449 | ** |
| 450 | ** |
| 451 | *** |
| 452 | *** |
| 453 | ** |
| 454 | ** |
| 455 | ** |

Without regard to whether a document cited herein was specifically and individually indicated as being incorporated by reference, all documents referred to herein are incorporated by reference into the present application for any and all purposes to the same extent as if each individual reference was fully set forth herein.

Having now fully described the subject matter of the claims, it will be understood by those having ordinary skill in the art that the same can be performed within a wide range of equivalents without affecting the scope of the subject matter or particular aspects described herein. It is intended that the appended claims be interpreted to include all such equivalents.

1. A compound of Formula (I): 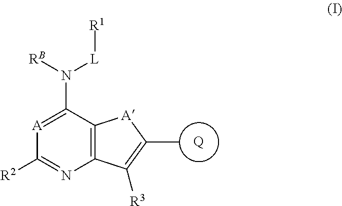
or a form thereof, wherein
A is selected from the group consisting of CR^{A} and N;
A′ is selected from the group consisting of S and NR^{A′};
L is selected from the group consisting of CH₂ and CD₂;
R^{A} is selected from the group consisting of hydrogen, halo, C₁₋₆alkyl, and C₃₋₈cycloalkyl;
R^{A′} is selected from the group consisting of hydrogen, C₁₋₄alkyl, and halo-C₁₋₄alkyl;
R^{B} is selected from the group consisting of hydrogen and C₁₋₆alkyl;
R¹ is selected from the group consisting of phenyl, heteroaryl, C₃₋₈cycloalkyl, CO₂C₁₋₆alkyl, C₂₋₆alkenyl, and C₂₋₆alkynyl;
wherein heteroaryl is a 5-11 membered monocyclic or bicyclic aromatic carbon atom ring structure radical containing 1-3 heteroatoms selected from N, O, and S;
wherein C₃₋₈cycloalkyl is a saturated or partially unsaturated monocyclic or bicyclic ring system;
wherein phenyl, heteroaryl, and C₃₋₈cycloalkyl are substituted with zero, one, two, three, or four, independently selected R^{1a} substituents,
R^{1a} is selected from the group consisting of cyano, halo, hydroxy, C₁₋₆alkyl, halo-C₁₋₆alkyl, deutero-C₁₋₆alkyl, and C₁₋₆alkoxy;
R² is selected from the group consisting of hydrogen, cyano, halo, C₂₋₆alkynyl, C₁₋₆alkoxy-C₂₋₆alkynyl, and hydroxy-C₂₋₆alkynyl;
R³ is selected from the group consisting of hydrogen, cyano, halo, hydroxy, SH, C₁₋₆alkyl, halo-C₁₋₆alkyl, hydroxy-C₁₋₆alkyl, C₁₋₆alkoxy, halo-C₁₋₆alkoxy, thio-C₁₋₆alkyl, C₂₋₆alkenyl, C₂₋₆alkynyl, C₁₋₆alkoxy-C₂₋₆alkynyl, hydroxy-C₂₋₆alkynyl, (CH₃)₃Si—C₂₋₆alkynyl, heteroaryl-C₂₋₆alkynyl, C₃₋₈cycloalkyl, phenyl, and heteroaryl,
wherein heteroaryl is a 5-11 membered monocyclic or bicyclic aromatic carbon atom ring structure radical containing 1-3 heteroatoms selected from N, C, and S, and
wherein each instance of C₃₋₈cycloalkyl, phenyl, and heteroaryl are independently substituted with zero, one, two, three, or four, independently selected R^{3a} substituents;
R^{3a} is selected from the group consisting of cyano, halo, hydroxy, C₁₋₆alkyl, halo-C₁₋₆alkyl, hydroxy-C₁₋₆alkyl, deutero-C₁₋₆alkyl, and C₁₋₆alkoxy;
Ring Q is 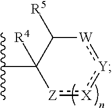
R⁴ is selected from the group consisting of hydrogen, cyano, halo, hydroxy, C₁₋₆alkyl, halo-C₁₋₆alkyl, C₁₋₆alkoxy, and halo-C₁₋₆alkoxy;
R⁵ is selected from the group consisting of amino, C₁₋₄alkyl-amino, and (C₁₋₄alkyl)₂-amino;
W is selected from the group consisting of CH₂, CD₂, CH—R^{w}, CD-R^{w}, C(R^{w})₂, and C(O);
X is selected from the group consisting of CH₂, CD₂, CH—R^{X}, CD-R^{X}, C(R^{X})₂, CH, CD, CR^{X}, C═CH₂, C═CD₂, C═C(R^{X})₂, C(O), NH, N—C₁₋₄alkyl, N-phenyl, O, S, S(O), and SO₂;
Y is selected from the group consisting of CH₂, CD₂, CH—R^{Y}, CD-R^{Y}, C(R^{Y})₂, CH, CD, CR^{Y}, C═CH₂, C═CD₂, C═C(R^{Y})₂, N-phenyl, O, S, S(O), and SO₂;
Z is selected from the group consisting of C₁₋₂, CD₂, CH—R^{Z}, CD-R^{Z}, C(R^{Z})₂, CH, CD, CR^{Z}, NH, N—C₁₋₄alkyl, N-phenyl, O, S, S(O), and SO₂;
each R^{W}, R^{X}, R^{Y}, R^{Z} are independently selected from the group consisting of halo, hydroxy, C₁₋₆alkyl, halo-C₁₋₆alkyl, C₁₋₆alkoxy, halo-C₁₋₆alkoxy, amino, C₁₋₄alkyl-amino, (C₁₋₄alkyl)₂-amino, CO₂H, CO₂C₁₋₆alkyl, C(O)NH₂, C(O)N(C₁₋₆alkyl)₂, C(O)-heterocyclyl, and C(O)NH-phenyl, and wherein each R^{W}, R^{X}, R^{Y}, R^{Z} may combine to form a carbocyclic or heterocyclic ring;
n is selected from the group consisting of 0, 1, 2, and 3; and [symbol]
independently represents a single bond or a double bond as valency permits; wherein the form of the compound is a pharmaceutically acceptable salt, hydrate, solvate, racemate, enantiomer, diastereomer, stereoisomer, tautomer or isotope enriched form thereof.

2. The compound of claim 1, wherein the compound is a compound of Formula (Ia): 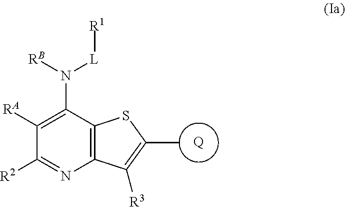
or a form thereof, wherein
L is CH₂ or CD;
R¹ is hydrogen;
R^{B} is hydrogen or CH₃; and
R² is Cl or CN.

3. The compound of claim 1, wherein the compound is a compound of Formula (Ib): 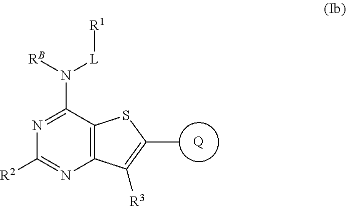
or a form thereof, wherein
L is CH₂;
R^{B} is hydrogen, and
R² is Cl.

4. The compound of claim 1, wherein the compound is a compound of Formula (Ic): 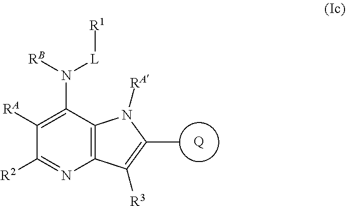
or a form thereof, wherein
L is CH₂;
R^{A} is hydrogen;
R^{A′} is CH₃ or CHF₂;
R^{B} is hydrogen; and
R² is Cl.

5. The compound of claim 1, wherein the compound is a compound of Formula (Id): 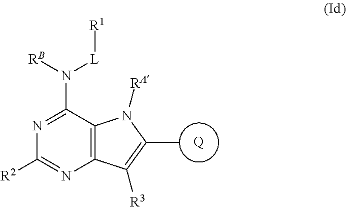
or a form thereof, wherein
L is CH₂;
R^{A′} is CHF₂:
R^{B} is hydrogen; and
R² is Cl.

6. The compound of claim 1, wherein R¹ is phenyl, CO₂CH₃,

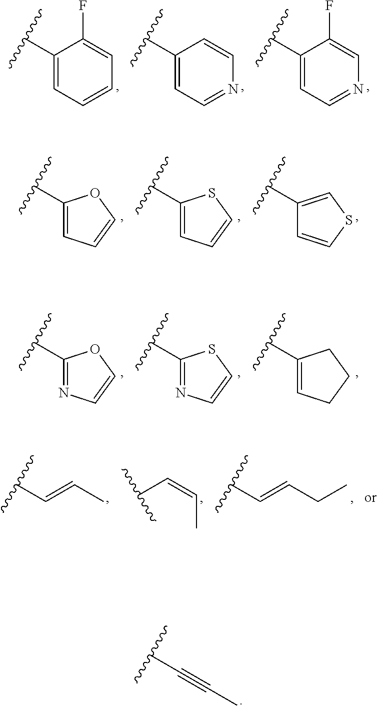

7. The compound of claim 1, wherein R³ is hydrogen, cyano, Cl, Br, I, CH₃, CH₂CH₃, CH₂F, CHF₂, CF₃, CH₂CH₂F, CH₂OH, CH₂OCH F₂, CH═CH₂, CH═C═CH₂, cyclopropyl, phenyl,

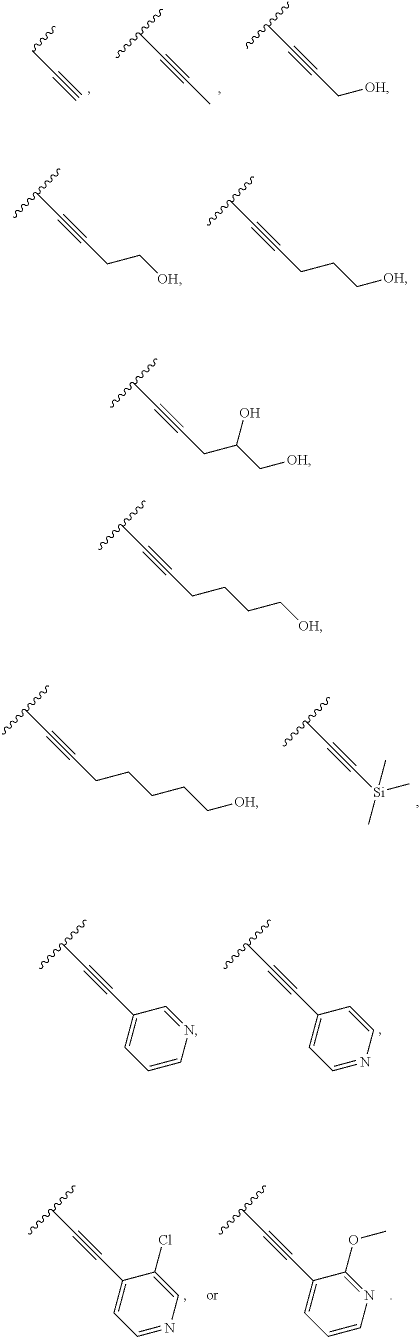

8. The compound of claim 1, wherein Q is: 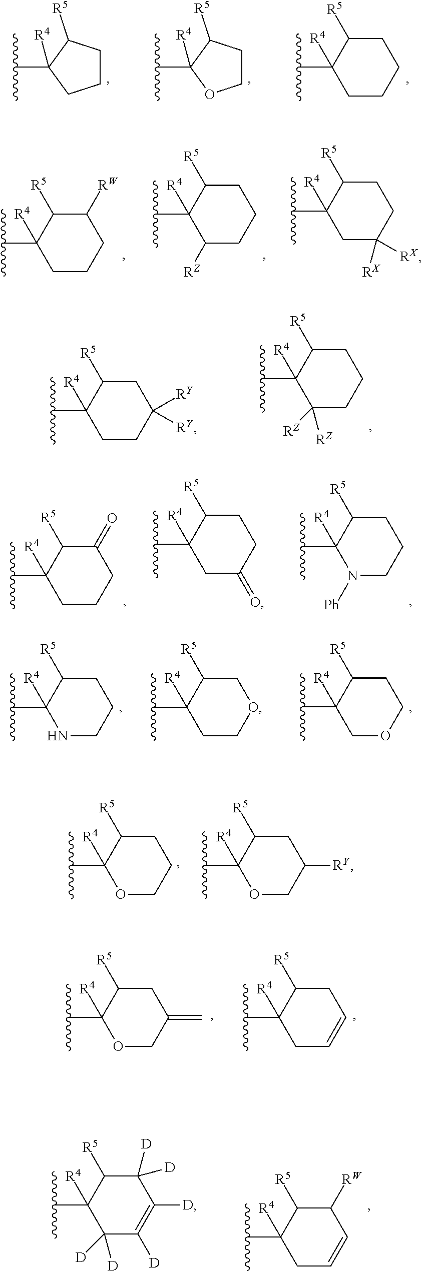 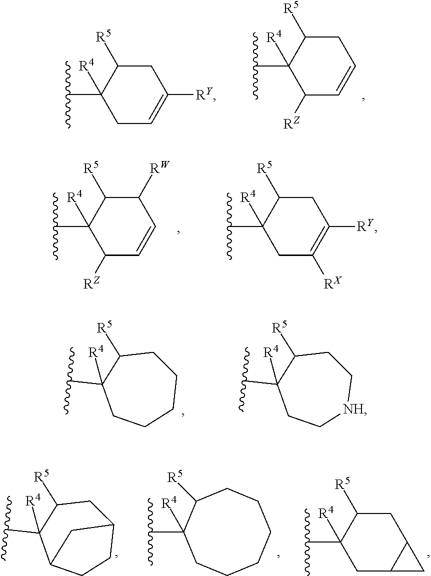
or any stereoisomers forms thereof.

9. The compound of claim 1, wherein
R⁴ is hydrogen, F, or hydroxy; and
R⁵ is amino, NH(CH₃), or N(CH₃)₂.

10. The compound of claim 1, wherein R^{W}, R^{X}, R^{Y}, R^{Z} are each independently selected from F, hydroxy, CH₃, OCH₃, CO₂H, CO₂CH₂CH₃, C(O)NH₂, C(O)N(CH₃)₂,

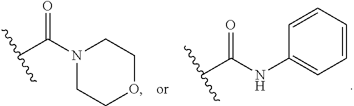 [SMILES: *C(=O)N1CCOCC1 ; *C(=O)Nc1ccccc1]

11. A compound, or a form thereof, wherein the compound is selected from the group consisting of:
2-((1S,2S)-2-aminocyclopentyl)-3,5-dichloro-N-(thiophen-2-ylmethyl)thieno[3,2-b]pyridin-7-amine;
2-((1R,2R)-2-aminocyclopentyl)-3,5-dichloro-N-(thiophen-2-ylmethyl)thieno[3,2-b]pyridin-7-amine;
2-((1S,2R)-2-aminocyclopentyl)-3,5-dichloro-N-(thiophen-2-ylmethyl)thieno[3,2-b]pyridin-7-amine;
2-((1R,2S)-2-aminocyclopentyl)-3,5-dichloro-N-(thiophen-2-ylmethyl)thieno[3,2-b]pyridin-7-amine;
2-((1R,2R)-2-aminocyclopentyl)-5-chloro-3-methyl-N-(thiophen-2-ylmethyl)thieno[3,2-b]pyridin-7-amine;
2-((1S,2S)-2-aminocyclopentyl)-5-chloro-3-methyl-N-(thiophen-2-ylmethyl)thieno[3,2-b]pyridin-7-amine;
2-((1R,2R)-2-aminocyclopentyl)-3-bromo-5-chloro-N-(thiophen-2-ylmethyl)thieno[3,2-b]pyridin-7-amine;
2-((1S,2S)-2-aminocyclopentyl)-3-bromo-5-chloro-N-(thiophen-2-ylmethyl)thieno[3,2-b]pyridin-7-amine;
2-((1S,2R)-2-aminocyclopentyl)-5-chloro-N-(thiophen-2-ylmethyl)thieno[3,2-b]pyridin-7-amine;
2-((1S,2S)-2-aminocyclopentyl)-5-chloro-N-(thiophen-2-ylmethyl)thieno[3,2-b]pyridin-7-amine;
2-((1S,2S)-2-aminocyclopentyl)-5-chloro-3-ethyl-N-(thiophen-2-ylmethyl)thieno[3,2-b]pyridin-7-amine;
2-((1R,2R)-2-aminocyclopentyl)-5-chloro-3-ethyl-N-(thiophen-2-ylmethyl)thieno[3,2-b]pyridin-7-amine;
2-((1R,2S)-2-aminocyclohexyl)-3,5-dichloro-N-(thiophen-2-ylmethyl)thieno[3,2-b]pyridin-7-amine;
2-((1S,2R)-2-aminocyclohexyl)-3,5-dichloro-N-(thiophen-2-ylmethyl)thieno[3,2-b]pyridin-7-amine;
2-((2S,3R)-3-aminotetrahydro-2H-pyran-2-yl)-3-bromo-5-chloro-N-(thiophen-2-ylmethyl)thieno[3,2-b]pyridin-7-amine;
2-((2R,3R)-3-aminotetrahydro-2H-pyran-2-yl)-3,5-dichloro-N-(thiophen-2-ylmethyl)thieno[3,2-b]pyridin-7-amine;
2-((2S,3R)-3-aminotetrahydro-2H-pyran-2-yl)-3,5-dichloro-N-(thiophen-2-ylmethyl)thieno[3,2-b]pyridin-7-amine;
2-[(2S,3S)-3-aminotetrahydropyran-2-yl]-3,5-dichloro-N-(2-thienylmethyl)thieno[3,2-b]pyridin-7-amine;
2-((2R,3S)-3-aminotetrahydro-2H-pyran-2-yl)-3,5-dichloro-N-(thiophen-2-ylmethyl)thieno[3,2-b]pyridin-7-amine;
3-bromo-5-chloro-2-[(2R,3S)-3-aminotetrahydropyran-2-yl]-N-(2-thienylmethyl)thieno[3,2-b]pyridin-7-amine;
3-bromo-5-chloro-2-[(2S,3R)-3-aminotetrahydropyran-2-yl]-N-(2-thienylmethyl)thieno[3,2-b]pyridin-7-amine;
2-((1S,2S)-2-aminocyclohexyl)-5-chloro-N-(thiophen-2-ylmethyl)thieno[3,2-b]pyridin-7-amine;
2-((1R,2S)-2-aminocyclohexyl)-5-chloro-N-(thiophen-2-ylmethyl)thieno[3,2-b]pyridin-7-amine;
2-((1R,2R)-2-aminocyclohexyl)-5-chloro-N-(thiophen-2-ylmethyl)thieno[3,2-b]pyridin-7-amine;
2-((2R,3R)-3-aminotetrahydro-2H-pyran-2-yl)-3,5-dichloro-N-(2-fluorobenzyl)thieno[3,2-b]pyridin-7-amine;
2-((2 S, 3S)-3-aminotetrahydro-2H-pyran-2-yl)-3,5-dichloro-N-(2-fluorobenzyl)thieno[3,2-b]pyridin-7-amine;
2-((2R,3R)-3-aminotetrahydro-2H-pyran-2-yl)-5-chloro-N-(furan-2-ylmethyl)-3-m ethylthieno[3,2-b]pyridin-7-amine;
2-((2S,3S)-3-aminotetrahydro-2H-pyran-2-yl)-5-chloro-N-(furan-2-ylmethyl)-3-methylthieno[3,2-b]pyridin-7-amine;
2-((2R,3R)-3-aminotetrahydro-2H-pyran-2-yl)-N-benzyl-5-chlorothieno[3,2-b]pyridin-7-amine;
2-((2S,3S)-3-aminotetrahydro-2H-pyran-2-yl)-N-benzyl-5-chlorothieno[3,2-b]pyridin-7-amine;
2-(3-aminotetrahydro-2H-pyran-2-yl)-N-benzyl-3-bromo-5-chlorothieno[3,2-b]pyridin-7-amine;
2-((2R,3R)-3-aminotetrahydro-2H-pyran-2-yl)-5-chloro-N-(2-fluorobenzyl)thieno[3,2-b]pyridin-7-amine;
2-((2S,3S)-3-aminotetrahydro-2H-pyran-2-yl)-5-chloro-N-(2-fluorobenzyl)thieno[3,2-b]pyridin-7-amine;
2-((2R,3R)-3-aminotetrahydro-2H-pyran-2-yl)-3-bromo-5-chloro-N-(2-fluorobenzyl)thieno[3,2-b]pyridin-7-amine;
2-((2S,3S)-3-aminotetrahydro-2H-pyran-2-yl)-3-bromo-5-chloro-N-(2-fluorobenzyl)thieno[3,2-b]pyridin-7-amine;
2-((1R,2S)-2-amino-4,4-difluorocyclohexyl)-3-bromo-5-chloro-N-(thiophen-2-ylmethyl)thieno[3,2-b]pyridin-7-amine;
2-((1S,2R)-2-amino-4,4-difluorocyclohexyl)-3-bronc-5-chloro-N-(thiophen-2-ylmethyl)thieno[3,2-b]pyridin-7-amine;
(1R,2S)-2-amino-1-(3,5-dichloro-7-((thiophen-2-ylmethyl)amino)thieno[3,2-b]pyridin-2-yl)cyclohexan-1-ol;
2-((1R,2S)-2-amino-1-fluorocyclohexyl)-3,5-dichloro-N-(thiophen-2-ylmethyl)thieno[3,2-b]pyridin-7-amine;
2-((2S,3S)-3-aminotetrahydro-2H-pyran-2-yl)-5-chloro-N-(2-fluorobenzyl)-3-methylthieno[3,2-b]pyridin-7-amine;
2-((1S,2S)-2-aminocyclohexyl)-3,5-dichloro-N-(thiophen-2-ylmethyl)thieno[3,2-b]pyridin-7-amine;
2-((1R,2R)-2-aminocyclohexyl)-3,5-dichloro-N-(thiophen-2-ylmethyl)thieno[3,2-b]pyridin-7-amine;
2-((1R,2R)-2-aminocyclohexyl)-5-chloro-3-methyl-N-(thiophen-2-ylmethyl)thieno[3,2-b]pyridin-7-amine;
2-((1S,2S)-2-aminocyclohexyl)-5-chloro-3-methyl-N-(thiophen-2-ylmethyl)thieno[3,2-b]pyridin-7-amine;
2-((1R,2R)-2-aminocyclohexyl)-3,5-dichloro-N-(thiophen-2-ylmethyl)thieno[3,2-b]pyridin-7-amine;
2-((1S,2S)-2-aminocyclohexyl)-3,5-dichloro-N-(thiophen-2-ylmethyl)thieno[3,2-b]pyridin-7-amine;
2-((1R,2S)-2-amino-1-fluorocyclohexyl)-N-benzyl-3-bromo-5-chlorothieno[3,2-b]pyridin-7-amine;
2-((1R,2S)-2-amino-1-fluorocyclohexyl)-3,5-dichloro-N-(furan-2-ylmethyl)thieno[3,2-b]pyridin-7-amine;
2-((1R,2R)-2-aminocyclohexyl)-5-chloro-3-ethyl-N-(thiophen-2-ylmethyl)thieno[3,2-b]pyridin-7-amine;
2-((1S,2S)-2-aminocyclohexyl)-5-chloro-3-ethyl-N-(thiophen-2-ylmethyl)thieno[3,2-b]pyridin-7-amine;
2-((1S,2)-2-aminocyclohexyl)-5-chloro-3-ethyl-N-(thiophen-2-ylmethyl)thieno[3,2-b]pyridin-7-amine;
2-((1R,2R)-2-aminocyclohexyl)-5-chloro-3-ethyl-N-(thiophen-2-ylmethyl)thieno[3,2-b]pyridin-7-amine;
2-((1R,2S)-2-amino-1-fluorocyclohexyl)-5-chloro-N-(furan-2-ylmethyl)-3-methylthieno[3,2-b]pyridin-7-amine;
6-((2R,3R)-3-aminotetrahydro-2H-pyran-2-yl)-7-bromo-2-chloro-N-(furan-2-ylmethyl)thieno[3,2-d]pyrimidin-4-amine;
6-((2S,3S)-3-aminotetrahydro-2H-pyran-2-yl)-7-bromo-2-chloro-N-(furan-2-ylmethyl)thieno[3,2-d]pyrimidin-4-amine;
6-((1R,2R)-2-aminocyclohexyl)-N-benzyl-2-chloro-7-methylthieno[3,2-d]pyrimidin-4-amine;
6-((1S,2S)-2-aminocyclohexyl)-N-benzyl-2-chloro-7-methylthieno[3,2-d]pyrimidin-4-amine;
2-((2R,3R)-3-aminotetrahydro-2H-pyran-2-yl)-5-chloro-3-methyl-N-(thiophen-2-ylmethyl)thieno[3,2-b]pyridin-7-amine;
2-((2S,3S)-3-aminotetrahydro-2H-pyran-2-yl)-5-chloro-3-methyl-N-(thiophen-2-ylmethyl)thieno[3,2-]pyridin-7-amine;
2-((2R,3R)-3-aminotetrahydro-2H-pyran-2-yl)-5-chloro-3-methyl-N-(thiophen-2-ylmethyl)thieno[3,2-b]pyridin-7-amine;
2-((2S,3S)-3-aminotetrahydro-2H-pyran-2-yl)-5-chloro-3-methyl-N-(thiophen-2-ylmethyl)thieno[3,2-b]pyridin-7-amine;
2-((2R,3S)-3-aminopiperidin-2-yl)-3-bromo-5-chloro-N-(thiophen-2-ylmethyl)thieno[3,2-b]pyridin-7-amine;
2-((2S,3R)-3-aminopiperidin-2-yl)-3-bromo-5-chloro-N-(thiophen-2-ylmethyl)thieno[3,2-b]pyridin-7-amine;
2-((2R,3R)-3-aminopiperidin-2-yl)-3-bromo-5-chloro-N-(thiophen-2-ylmethyl)thieno[3,2-b]pyridin-7-amine;
2-((2S,3S)-3-aminopiperidin-2-yl)-3-bromo-5-chloro-N-(thiophen-2-ylmethyl)thieno[3,2-b]pyridin-7-amine;
6-((1R,2R)-2-aminocyclohexyl)-N-benzyl-2-chloro-7-methylthieno[3,2-d]pyrimidin-4-amine;
6-((1S,2S)-2-aminocyclohexyl)-N-benzyl-2-chloro-7-methylthieno[3,2-d]pyrimidin-4-amine;
2-((2R,3R)-3-aminotetrahydro-2H-pyran-2-yl)-3-bromo-5-chloro-N-(thiophen-2-ylmethyl)thieno[3,2-b]pyridin-7-amine;
2-((1R,2S)-2-aminocyclohexyl)-5-chloro-3-methyl-N-(thiophen-2-ylmethyl)thieno[3,2-b]pyridin-7-amine;
2-((1S,2R)-2-aminocyclohexyl)-5-chloro-3-methyl-N-(thiophen-2-ylmethyl)thieno[3,2-b]pyridin-7-amine;
2-((1S,2R)-2-aminocyclohexyl)-3-bromo-5-chloro-N-(thiophen-2-ylmethyl)thieno[3,2-b]pyridin-7-amine;
2-((1R,2S)-2-aminocyclohexyl)-3-bromo-5-chloro-N-(thiophen-2-ylmethyl)thieno[3,2-b]pyridin-7-amine;
2-((1R,2R)-2-aminocyclohexyl)-3-bromo-5-chloro-N-(thiophen-2-ylmethyl)thieno[3,2-b]pyridin-7-amine;
2-((1S,2S)-2-aminocyclohexyl)-3-bromo-5-chloro-N-(thiophen-2-ylmethyl)thieno[3,2-b]pyridin-7-amine;
2-((2S,3S)-3-aminotetrahydro-2H-pyran-2-yl)-3-bromo-5-chloro-N-(thiophen-2-ylmethyl)thieno[3,2-b]pyridin-7-amine;
2-((1S,2S)-2-aminocyclohexyl)-5-chloro-3-iodo-N-(thiophen-2-ylmethyl)thieno[3,2-b]pyridin-7-amine;
2-((1R,2R)-2-aminocyclohexyl)-5-chloro-3-iodo-N-(thiophen-2-ylmethyl)thieno[3,2-b]pyridin-7-amine;
6-((1R,2R)-2-aminocyclohexyl)-2-chloro-N-(furan-2-ylmethyl)-7-methylthieno[3,2-d]pyrimidin-4-amine;
6-((1S,2S)-2-aminocyclohexyl)-2-chloro-N-(furan-2-ylmethyl)-7-methylthieno[3,2-d]pyrimidin-4-amine;
6-((2R,3R)-3-aminotetrahydro-2H-pyran-2-yl)-2-chloro-N-(furan-2-ylmethyl)-7-methylthieno[3,2-d]pyrimidin-4-amine;
6-((2S,3S)-3-aminotetrahydro-2H-pyran-2-yl)-2-chloro-N-(furan-2-ylmethyl)-7-methylthieno[3,2-d]pyrimidin-4-amine;
6-((2S,3S)-3-aminotetrahydro-2H-pyran-2-yl)-N-benzyl-2-chloro-7-methylthieno[3,2-d]pyrimidin-4-amine;
6-((2R,3R)-3-aminotetrahydro-2H-pyran-2-yl)-N-benzyl-2-chloro-7-methylthieno[3,2-d]pyrimidin-4-amine;
6-[(1S,2S)-2-aminocyclohexyl]-N-benzyl-7-bromo-2-chloro-thieno[3,2-d]pyrimidin-4-amine;
6-((1R,2R)-2-aminocyclohexyl)-N-benzyl-7-bromo-2-chlorothieno[3,2-d]pyrimidin-4-amine;
6-((1R,2R)-2-aminocyclohexyl)-7-bromo-2-chloro-N-(furan-2-ylmethyl)thieno[3,2-d]pyrimidin-4-amine;
6-((1S,2S)-2-aminocyclohexyl)-7-bromo-2-chloro-N-(furan-2-ylmethyl)thieno[3,2-d]pyrimidin-4-amine;
6-((2S,3S)-3-aminotetrahydro-2H-pyran-2-yl)-N-benzyl-7-bromo-2-chlorothieno[3,2-d]pyrimidin-4-amine;
6-((2R,3R)-3-aminotetrahydro-2H-pyran-2-yl)-N-benzyl-7-bromo-2-chlorothieno[3,2-d]pyrimidin-4-amine;
2-((1S,2S)-2-aminocyclohexyl)-5-chloro-3-methyl-N-(thiophen-2-ylmethyl)thieno[3,2-b]pyridin-7-amine;
2-((1S,2R)-2-aminocycloheptyl)-3-bromo-5-chloro-N-(thiophen-2-ylmethyl)thieno[3,2-b]pyridin-7-amine;
2-((1R,2S)-2-aminocycloheptyl)-3-bromo-5-chloro-N-(thiophen-2-ylmethyl)thieno[3,2-b]pyridin-7-amine;
2-((1R,2R)-2-aminocycloheptyl)-3-bromo-5-chloro-N-(thiophen-2-ylmethyl)thieno[3,2-b]pyridin-7-amine;
2-((1S,2S)-2-aminocycloheptyl)-3-bromo-5-chloro-N-(thiophen-2-ylmethyl)thieno[3,2-b]pyridin-7-amine;
2-((1S,2R)-2-aminocycloheptyl)-3,5-dichloro-N-(thiophen-2-ylmethyl)thieno[3,2-b]pyridin-7-amine;
2-((1R,2S)-2-aminocycloheptyl)-3,5-dichloro-N-(thiophen-2-ylmethyl)thieno[3,2-b]pyridin-7-amine;
2-((1R,2R)-2-aminocycloheptyl)-3,5-dichloro-N-(thiophen-2-ylmethyl)thieno[3,2-b]pyridin-7-amine;
2-((1S,2S)-2-aminocycloheptyl)-3,5-dichloro-N-(thiophen-2-ylmethyl)thieno[3,2-b]pyridin-7-amine;
2-((1R,2R)-2-aminocycloheptyl)-5-chloro-N-(thiophen-2-ylmethyl)thieno[3,2-b]pyridin-7-amine;
2-((1S,2S)-2-aminocycloheptyl)-5-chloro-3-methyl-N-(thiophen-2-ylmethyl)thieno[3,2-b]pyridin-7-amine;
2-((1R,2S)-2-aminocycloheptyl)-5-chloro-3-methyl-N-(thiophen-2-ylmethyl)thieno[3,2-b]pyridin-7-amine;
2-((1R,2R)-2-aminocycloheptyl)-5-chloro-3-methyl-N-(thiophen-2-ylmethyl)thieno[3,2-b]pyridin-7-amine;
2-((1S,2R)-2-aminocycloheptyl)-5-chloro-3-methyl-N-(thiophen-2-ylmethyl)thieno[3,2-b]pyridin-7-amine;
2-((2R,3R)-3-aminobicyclo[3.2.1]octan-2-yl)-3-bromo-5-chloro-N-(thiophen-2-ylmethyl)thieno[3,2-b]pyridin-7-amine;
2-((2S,3S)-3-aminobicyclo[3.2.1]octan-2-yl)-3-bromo-5-chloro-N-(thiophen-2-ylmethyl)thieno[3,2-b]pyridin-7-amine;
2-((1R,2R)-2-aminocycloheptyl)-5-chloro-3-ethyl-N-(thiophen-2-ylmethyl)thieno[3,2-b]pyridin-7-amine;
2-((1S,2S)-2-aminocycloheptyl)-5-chloro-3-ethyl-N-(thiophen-2-ylmethyl)thieno[3,2-b]pyridin-7-amine;
2-((1S,2S)-2-aminocycloheptyl)-5-chloro-3-ethyl-N-(thiophen-2-ylmethyl)thieno[3,2-b]pyridin-7-amine;
2-((1R,2R)-2-aminocycloheptyl)-5-chloro-3-ethyl-N-(thiophen-2-ylmethyl)thieno[3,2-b]pyridin-7-amine;
2-((1S,2R)-2-aminocycloheptyl)-5-chloro-N-(thiophen-2-ylmethyl)thieno[3,2-b]pyridin-7-amine;
2-((1R,2S)-2-aminocycloheptyl)-5-chloro-N-(thiophen-2-ylmethyl)thieno[3,2-b]pyridin-7-amine;
2-((1S,2S)-2-aminocycloheptyl)-5-chloro-N-(thiophen-2-ylmethyl)thieno[3,2-b]pyridin-7-amine;
2-(1R,2S)-2-aminocyclooctyl)-3,5-dichloro-N-(thiophen-2-ylmethyl)thieno[3,2-b]pyridin-7-amine;
2-((1R,2R)-2-aminocyclooctyl)-5-chloro-3-methyl-N-(thiophen-2-ylmethyl)thieno[3,2-b]pyridin-7-amine;
2-((1S,2R)-2-aminocyclooctyl)-5-chloro-3-methyl-N-(thiophen-2-ylmethyl)thieno[3,2-b]pyridin-7-amine;
2-((1S,2S)-2-aminocyclooctyl)-5-chloro-3-methyl N-(thiophen-2-ylmethyl)thieno[3,2-b]pyridin-7-amine 2-((1R,2S)-2-aminocyclooctyl)-5-chloro-3-methyl-N-(thiophen-2-ylmethyl)thieno[3,2-b]pyridin-7-amine;
2-((1S,2S)-2-aminocyclooctyl)-3,5-dichloro-N-(thiophen-2-ylmethyl)thieno[3,2-b]pyridin-7-amine;
2-((1R,2R)-2-aminocyclooctyl)-3,5-dichloro-N-(thiophen-2-ylmethyl)thieno[3,2-b]pyridin-7-amine;
2-((1S,2R)-2-aminocyclooctyl)-3,5-dichloro-N-(thiophen-2-ylmethyl)thieno[3,2-b]pyridin-7-amine;
2-((1S,2S)-2-aminocyclooctyl)-3-bromo-5-chloro-N-(thiophen-2-ylmethyl)thieno[3,2-b]pyridin-7-amine;
2-((1R,2R)-2-aminocyclooctyl)-3-bromo-5-chloro-N-(thiophen-2-ylmethyl)thieno[3,2-b]pyridin-7-amine;
2-((1S,2R)-2-aminocyclooctyl)-3-bromo-5-chloro-N-(thiophen-2-ylmethyl)thieno[3,2-b]pyridin-7-amine;
2-((1R,2S)-2-aminocyclooctyl)-3-bromo-5-chloro-N-(thiophen-2-ylmethyl)thieno[3,2-b]pyridin-7-amine;
2-((1S,2S)-2-aminocyclooctyl)-5-chloro-N-(thiophen-2-ylmethyl)thieno[3,2-b]pyridin-7-amine 2-((1R,2S)-2-aminocyclooctyl)-5-chloro-N-(thiophen-2-ylmethyl)thieno[3,2-b]pyridin-7-amine;
2-((1R,2R)-2-aminocyclooctyl)-5-chloro-N-(thiophen-2-ylmethyl)thieno[3,2-b]pyridin-7-amine;
2-((1S,2R)-2-aminocyclooctyl)-5-chloro-N-(thiophen-2-ylmethyl)thieno[3,2-b]pyridin-7-amine;
2-((1S,2S)-2-aminocyclopentyl)-N-benzyl-5-chloro-3-methylthieno[3,2-b]pyridin-7-amine;
2-((2S,3S)-3-aminotetrahydro-2H-pyran-2-yl)-N-benzyl-3-bromo-5-chlorothieno[3,2 b]pyridin-7-amine;
2-((2R,3R)-3-aminotetrahydro-2H-pyran-2-yl)-N-benzyl-5-chloro-3-methylthieno[3,2 b]pyridin-7-amine;
2-((2S,3S)-3-aminotetrahydro-2H-pyran-2-yl)-N-benzyl-3,5-dichlorothieno[3,2-b]pyridin-7-amine;
2-((1R,2S)-2-aminocyclopentyl)-5-chloro-N-(thiophen-2-ylmethyl)thieno[3,2-b]pyridin-7-amine;
2-((1R,2R)-2-aminocyclopentyl)-5-chloro-N-(thiophen-2-ylmethyl)thieno[3,2-b]pyridin-7-amine;
2-((2R,3R)-3-aminotetrahydro-2H-pyran-2-yl)-N-benzyl-3-bromo-5-chlorothieno[3,2-b]pyridin-7-amine;
2-((1R,2R)-2-aminocyclopentyl)-3,5-dichloro-N-(furan-2-ylmethyl)thieno[3,2-b]pyridin-7-amine;
2-((2S,3S)-3-aminotetrahydro-2H-pyran-2-yl)-3-bromo-5-chloro-N-(furan-2-ylmethyl)thieno[3,2-b]pyridin-7-amine;
2-((2 S, 3S)-3-aminotetrahydro-2H-pyran-2-yl)-3,5-dichloro-N-(furan-2-ylmethyl)thieno[3,2-b]pyridin-7-amine;
2-((1S,2S)-2-aminocyclopentyl)-3,5-dichloro-N-(furan-2-ylmethyl)thieno[3,2-b]pyridin-7-amine;
2-((2R,3R)-3-aminotetrahydro-2H-pyran-2-yl)-3-bromo-5-chloro-N-(furan-2-ylmethyl)thieno[3,2-b]pyridin-7-amine;
2-((2R,3R)-3-aminotetrahydro-2H-pyran-2-yl)-3,5-dichloro-N-(furan-2-ylmethyl)thieno[3,2-b]pyridin-7-amine;
2-((1S,2S)-2-aminocyclopentyl)-5-chloro-N-(furan-2-ylmethyl)-3-methylthieno[3,2-b]pyridin-7-amine;
2-((1R,2S)-2-amino-1-fluorocyclopentyl)-3,5-dichloro-N-(furan-2-ylmethyl)thieno[3,2-b]pyridin-7-amine;
2-((1S,2S)-2-aminocyclooctyl)-5-chloro-3-iodo-N-(thiophen-2-ylmethyl)thieno[3,2-b]pyridin-7-amine;
2-((1R,2R)-2-aminocyclooctyl)-5-chloro-3-iodo-N-(thiophen-2-ylmethyl)thieno[3,2-b]pyridin-7-amine;
2-((2R,3R)-3-amino-1-phenylpiperidin-2-yl)-3-bromo-5-chloro-N-(thiophen-2-ylmethyl)thieno[3,2-b]pyridin-7-amine;
2-((2S,3S)-3-amino-1-phenylpiperidin-2-yl)-3-bromo-5-chloro-N-(thiophen-2-ylmethyl)thieno[3,2-b]pyridin-7-amine;
2-((2R,3S)-3-amino-1-phenylpiperidin-2-yl)-3-bromo-5-chloro-N-(thiophen-2-ylmethyl)thieno[3,2-b]pyridin-7-amine;
2-((2S,3R)-3-amino-1-phenylpiperidin-2-yl)-3-bromo-5-chloro-N-(thiophen-2-ylmethyl)thieno[3,2-b]pyridin-7-amine;
2-((2R,3R)-3-aminotetrahydro-2H-pyran-2-yl)-N-benzyl-5-chloro-3-methylthieno[3,2-b]pyridin-7-amine;
(1R,5S,6R)-6-amino-5-(3,5-dichloro-7-((thiophen-2-ylmethyl)amino)thieno[3,2-b]pyridin-2-yl)cyclohex-2-en-1-ol;
(1S,5R,6S)-6-amino-5-(3,5-dichloro-7-((thiophen-2-ylmethyl)amino)thieno[3,2-b]pyridin-2-yl)cyclohex-2-en-1-ol;
6-((1R,2S)-2-amino-1-fluorocyclopentyl)-2-chloro-N-(furan-2-ylmethyl)-7-methylthieno[3,2-d]pyrimidin-4-amine;
2-((1S,2S)-2-aminocyclohexyl)-5-chloro-3-phenyl-N-(thiophen-2-ylmethyl)thieno[3,2-b]pyridin-7-amine;
2-(1S,2S)-2-aminocyclohexyl)-5-chloro-3-cyclopropyl-N-(thiophen-2-ylmethyl)thieno[3,2-b]pyridin-7-amine;
2-((1S,2S)-2-aminocyclopentyl)-5-chloro-3-phenyl-N-(thiophen-2-ylmethyl)thieno[3,2-b]pyridin-7-amine;
2-((2R,3R)-3-aminotetrahydro-2H-pyran-2-yl)-N-benzyl-5-chloro-3-iodothieno[3,2-b]pyridin-7-amine,
2-((2S,3S)-3-aminotetrahydro-2H-pyran-2-yl)-5-chloro-N-(furan-2-ylmethyl)-3-methylthieno[3,2-b]pyridin-7-amine;
((2R,3R)-3-aminotetrahydro-2H-pyran-2-yl)-5-chloro-N-(furan-2-ylmethyl)-3-methylthieno[3,2-b]pyridin-7-amine;
2-((1R,2R)-2-aminocyclopentyl)-5-chloro-3-phenyl-N-(thiophen-2-ylmethyl)thieno[3,2-b]pyridin-7-amine;
2-((2S,3S)-3-aminotetrahydro-2H-pyran-2-yl)-N-benzyl-5-chloro-3-iodothieno[3,2-b]pyridin-7-amine;
2-((1R,2R)-2-aminocyclohexyl)-5-chloro-3-cyclopropyl-N-(thiophen-2-ylmethyl)thieno[3,2-b]pyridin-7-amine;
2-((1R,2R)-2-aminocyclohexyl)-5-chloro-3-cyclopropyl-N-(thiophen-2-ylmethyl)thieno[3,2-b]pyridin-7-amine;
2-((3R,4S)-3-aminotetrahydro-2H-pyran-4-yl)-3-bromo-5-chloro-N-(thiophen-2-ylmethyl)thieno[3,2-b]pyridin-7-amine;
2-((3S,4R)-3-aminotetrahydro-2H-pyran-4-yl)-3-bromo-5-chloro-N-(thiophen-2-ylmethyl)thieno[3,2-b]pyridin-7-amine;
2-((1S,2S)-2-aminocycloheptyl)-5-chloro-3-phenyl-N-(thiophen-2-ylmethyl)thieno[3,2-b]pyridin-7-amine,
2-((1R,2R)-2-aminocycloheptyl)-5-chloro-3-phenyl-N-(thiophen-2-ylmethyl)thieno[3,2-b]pyridin-7-amine;
6-((2S,3S)-3-aminotetrahydro-2H-pyran-2-yl)-7-bromo-2-chloro-N-(furan-2-ylmethyl)thieno[3,2-d]pyrimidin-4-amine;
2-(1S,6RS)-6-aminocyclohex-3-en-1-yl)-3-bromo-5-chloro-N-(thiophen-2-ylmethyl)thieno[3,2-b]pyridin-7-amine;
2-((1R,6SR)-6-aminocyclohex-3-en-1-yl)-3-bromo-5-chloro-N-(thiophen-2-ylmethyl)thieno[3,2-b]pyridin-7-amine;
2-((1R,2R)-2-aminocyclopentyl)-5-chloro-N-(furan-2-ylmethyl)-3-methylthieno[3,2-b]pyridin-7-amine;
2-((3R,4R)-3-aminotetrahydro-2H-pyran-4-yl)-3-bromo-5-chloro-N-(thiophen-2-ylmethyl)thieno[3,2-b]pyridin-7-amine;
2-((3S,4S)-3-aminotetrahydro-2H-pyran-4-yl)-3-bromo-5-chloro-N-(thiophen-2-ylmethyl)thieno[3,2-b]pyridin-7-amine;
2-((4R,5R)-5-aminoazepan-4-yl)-3-bromo-5-chloro-N-(thiophen-2-ylmethyl)thieno[3,2-b]pyridin-7-amine;
2-((4S,5S)-5-aminoazepan-4-yl)-3-bromo-5-chloro-N-(thiophen-2-ylmethyl)thieno[3,2-b]pyridin-7-amine;
2-((4R,5S)-5-aminoazepan-4-yl)-3-bromo-5-chloro-N-(thiophen-2-ylmethyl)thieno[3,2-b]pyridin-7-amine;
2-((4S,5R)-5-aminoazepan-4-yl)-3-bromo-5-chloro-N-(thiophen-2-ylmethyl)thieno[3,2-b]pyridin-7-amine;
2-((1R,2R)-2-amino-5,5-dimethylcyclohexyl)-3-bromo-5-chloro-N-(thiophen-2-ylmethyl)thieno[3,2-b]pyridin-7-amine;
2-((1S,2S)-2-amino-5,5-dimethylcyclohexyl)-3-bromo-5-chloro-N-(thiophen-2-ylmethyl)thieno[3,2-b]pyridin-7-amine;
2-((2R,3S)-3-aminotetrahydrofuran-2-yl)-3-bromo-5-chloro-N-(thiophen-2-ylmethyl)thieno[3,2-b]pyridin-7-amine;
2-((2S,3R)-3-aminotetrahydrofuran-2-yl)-3-bromo-5-chloro-N-(thiophen-2-ylmethyl)thieno[3,2-b]pyridin-7-amine;
2-((2R,3R)-3-aminotetrahydrofuran-2-yl)-3-bromo-5-chloro-N-(thiophen-2-ylmethyl)thieno[3,2-b]pyridin-7-amine;
2-((2S,3S)-3-aminotetrahydrofuran-2-yl)-3-bromo-5-chloro-N-(thiophen-2-ylmethyl)thieno[3,2-b]pyridin-7-amine;
2-((1R,2R)-2-amino-4,4-dimethylcyclohexyl)-3-bromo-5-chloro-N-(thiophen-2-ylmethyl)thieno[3,2-b]pyridin-7-amine;
2-((1S,2S)-2-amino-4,4-dimethylcyclohexyl)-3-bromo-5-chloro-N-(thiophen-2-ylmethyl)thieno[3,2-b]pyridin-7-amine;
(1R,5S,6R)-6-amino-5-(3-bromo-5-chloro-7-((thiophen-2-ylmethyl)amino)thieno[3,2-b]pyridin-2-yl)cyclohex-2-en-1-ol;
(1S,5R,6S)-6-amino-5-(3-bromo-5-chloro-7-((thiophen-2-ylmethyl)amino)thieno[3,2-b]pyridin-2-yl)cyclohex-2-en-1-ol;
2-((1R,2R)-2-aminocyclopentyl)-5-chloro-3-iodo-N-(thiophen-2-ylmethyl)thieno[3,2-b]pyridin-7-amine;
2-((1R,2R)-2-aminocyclopentyl)-3-bromo-5-chloro-N-(furan-2-ylmethyl)thieno[3,2-b]pyridin-7-amine;
2-((2R,3R)-3-aminotetrahydro-2H-pyran-2-yl)-N-benzyl-3,5-dichlorothieno[3,2-b]pyridin-7-amine;
2-((1R,2R)-2-aminocycloheptyl)-5-chloro-3-iodo-N-(thiophen-2-ylmethyl)thieno[3,2-b]pyridin-7-amine;
2-((1R,2R)-2-aminocyclooctyl)-5-chloro-3-ethyl-N-(thiophen-2-ylmethyl)thieno[3,2-b]pyridin-7-amine;
2-((1S,2S)-2-aminocyclopentyl)-N-benzyl-3,5-dichlorothieno[3,2-b]pyridin-7-amine;
2-((1R,2R)-2-aminocyclopentyl)-N-benzyl-5-chloro-3-methylthieno[3,2-b]pyridin-7-amine;
2-((1R,2R)-2-aminocyclopentyl)-N-benzyl-3-bromo-5-chlorothieno[3,2-b]pyridin-7-amine;
2-((1S,2S)-2-aminocyclopentyl)-5-chloro-3-iodo-N-(thiophen-2-ylmethyl)thieno[3,2-b]pyridin-7-amine;
2-((1S,2S)-2-aminocycloheptyl)-5-chloro-3-iodo-N-(thiophen-2-ylmethyl)thieno[3,2-b]pyridin-7-amine;
2-((1S,2S)-2-aminocyclooctyl)-5-chloro-3-ethyl-N-(thiophen-2-ylmethyl)thieno[3,2-b]pyridin-7-amine;
2-((1S,2S)-2-aminocyclopentyl)-3-bromo-5-chloro-N-(furan-2-ylmethyl)thieno[3,2-b]pyridin-7-amine;
2-((1S,2S)-2-aminocyclopentyl)-N-benzyl-3-bromo-5-chlorothieno[3,2-b]pyridin-7-amine;
2-((1R,2R)-2-aminocyclopentyl)-N-benzyl-3,5-dichlorothieno[3,2-b]pyridin-7-amine;
6-((1S,6S)-6-aminocyclohex-3-en-1-yl)-7-bromo-2-chloro-N-(furan-2-ylmethyl)thieno[3,2-d]pyrimidin-4-amine;
2-((1S,6S)-6-aminocyclohex-3-en-1-yl)-3,5-dichloro-N-(furan-2-ylmethyl)thieno[3,2-b]pyridin-7-amine;
6-((1S,6S)-6-aminocyclohex-3-en-1-yl)-2,7-dichloro-N-(furan-2-ylmethyl)thieno[3,2-d]pyrimidin-4-amine;
2-((1R,6R)-6-aminocyclohex-3-en-1-yl-2,2,3,4,5,5-d6)-5-chloro-N-(furan-2-ylmethyl)-3-methylthieno[3,2-b]pyridin-7-amine;
2-((1S,6S)-6-aminocyclohex-3-en-1-yl-2,2,3,4,5,5-d6)-5-chloro-N-(furan-2-ylmethyl)-3-methylthieno[3,2-b]pyridin-7-amine;
6-((1S,6S)-6-aminocyclohex-3-en-1-yl)-2-chloro-N-(furan-2-ylmethyl)-7-iodothieno[3,2-d]pyrimidin-4-amine;
2-((1S,2S)-2-aminocyclohexyl)-3,5-dichloro-N-(furan-2-ylmethyl)thieno[3,2-b]pyridin-7-amine;
2-((3R,4S)-4-aminotetrahydro-2H-pyran-3-yl)-3,5-dichloro-N-(furan-2-ylmethyl)thieno[3,2-b]pyridin-7-amine;
5-(2-((1R,6R)-6-aminocyclohex-3-en-1-yl)-5-chloro-7-((thiophen-2-ylmethyl)amino)thieno[3,2-b]pyridin-3-yl)pent-4-yn-1-ol;
5-(2-((1S,6S)-6-aminocyclohex-3-en-1-yl)-5-chloro-7-((thiophen-2-ylmethyl)amino)thieno[3,2-b]pyridin-3-yl)pent-4-yn-1-ol;
2-((1S,6S)-6-aminocyclohex-3-en-1-yl)-5-chloro-3-iodo-N-(thiophen-2-ylmethyl)thieno[3,2-b]pyridin-7-amine;
4-(2-((1R,6R)-6-aminocyclohex-3-en-1-yl)-5-chloro-7-((thiophen-2-ylmethyl)amino)thieno[3,2-b]pyridin-3-yl)but-3-yn-1-ol;
4-(2-((1S,6S)-6-aminocyclohex-3-en-1-yl)-5-chloro-7-((thiophen-2-ylmethyl)amino)thieno[3,2-b]pyridin-3-yl)but-3-yn-1-ol;
2-((1S,6S)-6-aminocyclohex-3-en-1-yl)-3,5-dichloro-N-(thiazol-2-ylmethyl)thieno[3,2-b]pyridin-7-amine;
2-((3R,4S)-4-aminotetrahydro-2H-pyran-3-yl)-3,5-dichloro-N-(thiophen-2-ylmethyl)thieno[3,2-b]pyridin-7-amine;
2-((1R,6R)-6-aminocyclohex-3-en-1-yl-2,2,3,4,5,5-d6)-3,5-dichloro-N-(furan-2-ylmethyl)thieno[3,2-b]pyridin-7-amine;
2-((1S,6S)-6-aminocyclohex-3-en-1-yl-2,2,3,4,5,5-d6)-3,5-dichloro-N-(furan-2-ylmethyl)thieno[3,2-b]pyridin-7-amine;
2-((1R,2S,6R)-2-amino-6-fluorocyclohexyl)-3-bromo-5-chloro-N-(thiophen-2-ylmethyl)thieno[3,2-b]pyridin-7-amine;
2-((1S,2R,6S)-2-amino-6-fluorocyclohexyl)-3-bromo-5-chloro-N-(thiophen-2-ylmethyl)thieno[3,2-b]pyridin-7-amine;
6-((1S,6S)-6-aminocyclohex-3-en-1-yl)-2-chloro-7-iodo-N-(thiophen-2-ylmethyl)thieno[3,2-d]pyrimidin-4-amine;
6-((1S,6S)-6-aminocyclohex-3-en-1-yl-2,2,3,4,5,5-d6)-7-bromo-2-chloro-N-(thiophen-2-ylmethyl)thieno[3,2-d]pyrimidin-4-amine;
2-((1S,6S)-6-aminocyclohex-3-en-1-yl)-5-chloro-N-(furan-2-ylmethyl)-3-methylthieno[3,2-b]pyridin-7-amine;
2-((1R,6R)-6-aminocyclohex-3-en-1-yl)-3,5-dichloro-N-(thiophen-2-ylmethyl)thieno[3,2-b]pyridin-7-amine;
2-((1S,6S)-6-aminocyclohex-3-en-1-yl)-3,5-dichloro-N-(thiophen-2-ylmethyl)thieno[3,2-b]pyridin-7-amine;
2-((1S,6S)-6-aminocyclohex-3-en-1-yl)-3-bromo-5-chloro-N-(thiophen-2-ylmethyl)thieno[3,2-b]pyridin-7-amine;
6-((1S,6S)-6-aminocyclohex-3-en-1-yl-2,2,3,4,5,5-d6)-2-chloro-N-(furan-2-ylmethyl)-7-methylthieno[3,2-d]pyrimidin-4-amine;
2-((1S,6S)-6-aminocyclohex-3-en-1-yl)-5-chloro-N-(furan-2-ylmethyl)-3-iodothieno[3,2-b]pyridin-7-amine;
2-((1R,2R)-2-aminocyclohexyl)-5-chloro-3-(prop-1-yn-1-yl)-N-(thiophen-2-ylmethyl)thieno[3,2-b]pyridin-7-amine;
2-((1S,2S)-2-aminocyclohexyl)-5-chloro-3-(prop-1-yn-1-yl)-N-(thiophen-2-ylmethyl)thieno[3,2-b]pyridin-7-amine;
2-((1S,6S)-6-aminocyclohex-3-en-1-yl)-N-benzyl-5-chloro-3-iodothieno[3,2-b]pyridin-7-amine;
6-((1S,6S)-6-aminocyclohex-3-en-1-yl)-7-bromo-2-chloro-N-(thiophen-2-ylmethyl)thieno[3,2-d]pyrimidin-4-amine;
(2R,3S)-2-amino-3-(3-bromo-5-chloro-7-((thiophen-2-ylmethyl)amino)thieno[3,2-b]pyridin-2-yl)cyclohexan-1-one;
(2S,3R)-2-amino-3-(3-bromo-5-cloro-7-((thiophen-2-ylmethyl)amino)thieno[3,2-b]pyridin-2-yl)cyclohexan-1-one;
6-((1S,6S)-6-aminocyclohex-3-en-1-yl)-2-chloro-N-(furan-2-ylmethyl)-7-methylthieno[3,2-d]pyrimidin-4-amine;
2-((1S,6S)-6-aminocyclohex-3-en-1-yl)-N-benzyl-3-bromo-5-chlorothieno[3,2-b]pyridin-7-amine;
2-((1R,6R)-6-aminocyclohex-3-en-1-yl-2,2,3,4,5,5-d6)-3,5-dichloro-N-(furan-2-ylmethyl)thieno[3,2-b]pyridin-7-amine;
2-((1S,6S)-6-aminocyclohex-3-en-1-yl-2,2,3,4,5,5-d6)-3,5-dichloro-N-(furan-2-ylmethyl)thieno[3,2-b]pyridin-7-amine;
2-((1S,6S)-6-aminocyclohex-3-en-1-yl)-3,5-dichloro-N-(thiophen-2-ylmethyl)thieno[3,2-b]pyridin-7-amine;
2-((1S,2S)-2-aminocyclohexyl)-3-bromo-5-chloro-N-(pyridin-4-ylmethyl)thieno[3,2-b]pyridin-7-amine;
2-((1S,6S)-6-aminocyclohex-3-en-1-yl)-3-bromo-5-chloro-N-(pyridin-4-ylmethyl)thieno[3,2-b]pyridin-7-amine;
2-((1S,6S)-6-aminocyclohex-3-en-1-yl)-3,5-dichloro-N-(oxazol-2-ylmethyl)thieno[3,2-b]pyridin-7-amine;
6-((1R,6R)-6-aminocyclohex-3-en-1-yl-2,2,3,4,5,5-d6)-7-bromo-2-chloro-N-(furan-2-ylmethyl)thieno[3,2-d]pyrimidin-4-amine;
6-((1S,6S)-6-aminocyclohex-3-en-1-yl-2,2,3,4,5,5-d6)-7-bromo-2-chloro-N-(furan-2-ylmethyl)thieno[3,2-d]pyrimidin-4-amine;
6-((1R,6S)-6-amino-2,2-difluorocyclohexyl)-7-bromo-2-chloro-N-(furan-2-ylmethyl)thieno[3,2-d]pyrimidin-4-amine;
2-((1S,6S)-6-aminocyclohex-3-en-1-yl)-N-(but-2-yn-1-yl)-5-chloro-3-iodothieno[3,2-b]pyridin-7-amine;
2-((1S,6S)-6-aminocyclohex-3-en-1-yl)-3,5-dichloro-N-(pyridin-4-ylmethyl)thieno[3,2-b]pyridin-7-amine;
2-((3R,4S)-4-aminotetrahydro-2H-pyran-3-yl)-3-bromo-5-chloro-N-(thiophen-2-ylmethyl)thieno[3,2-b]pyridin-7-amine;
2-((3S,4R)-4-aminotetrahydro-2H-pyran-3-yl)-3-bromo-5-chloro-N-(thiophen-2-ylmethyl)thieno[3,2-b]pyridin-7-amine;
2-((1S,6S)-6-aminocyclohex-3-en-1-yl-2,2,3,4,5,5-d6)-5-chloro-3-methyl-N-(thiophen-2-ylmethyl)thieno[3,2-b]pyridin-7-amine;
2-((1S,6S)-6-aminocyclohex-3-en-1-yl)-3-bromo-5-chloro-N-(furan-2-ylmethyl)thieno[3,2-b]pyridin-7-amine;
2-((1S,6S)-6-aminocyclohex-3-en-1-yl)-N-benzyl-3,5-dichlorothieno[3,2-b]pyridin-7-amine;
2-((6R)-6-amino-2,2-difluorocyclohexyl)-5-chloro-3-(prop-1-yn-1-yl)-N-(thiophen-2-ylmethyl)thieno[3,2-b]pyridin-7-amine;
2-((6S)-6-amino-2,2-difluorocyclohexyl)-5-chloro-3-(prop-1-yn-1-yl)-N-(thiophen-2-ylmethyl)thieno[3,2-b]pyridin-7-amine;
2-((1R,6R)-6-aminocyclohex-3-en-1-yl)-5-chloro-3-iodo-N-(thiophen-2-ylmethyl)thieno[3,2-b]pyridin-7-amine;
2-((1S,6S)-6-aminocyclohex-3-en-1-yl)-5-chloro-3-iodo-N-(thiophen-2-ylmethyl)thieno[3,2-b]pyridin-7-amine;
2-((1S,2S)-2-aminocyclohexyl)-N-benzyl-5-chloro-3-ethynylthieno[3,2-b]pyridin-7-amine;
(R)-5-(2-((1S,6S)-6-aminocyclohex-3-en-1-yl)-5-chloro-7-((thiophen-2-ylmethyl)amino)thieno[3,2-b]pyridin-3-yl)pent-4-yne-1,2-diol;
2-((1S,6S)-6-aminocyclohex-3-en-1-yl-2,2,3,4,5,5-d6)-5-chloro-N′-(furan-2-ylmethyl)-3-methylthieno[3,2-b]pyridin-7-amine;
4-(2-((2R,3R)-3-aminotetrahydro-2H-pyran-2-yl)-5-chloro-7-((thiophen-2-ylmethyl)amino)thieno[3,2-b]pyridin-3-yl)but-3-yn-1-ol;
4-(2-((2S,3S)-3-aminotetrahydro-2H-pyran-2-yl)-5-chloro-7-((thiophen-2-ylmethyl)amine)thieno[3,2-b]pyridin-3-yl)but-3-yn-1-cl;
6-((1S,6S)-6-aminocyclohex-3-en-1-yl)-2,7-dichloro-N-(pyridin-4-ylmethyl)thieno[3,2-d]pyrimidin-4-amine;
6-((2S,3S)-3-aminotetrahydro-2H-pyran-2-yl)-7-bromo-2-chloro-5-(difluoromethyl)-N-(furan-2-ylmethyl)-5H-pyrrolo[3,2-d]pyrimidin-4-amine;
(S)-5-(2-((1S,6S)-6-aminocyclohex-3-en-1-yl)-5-chloro-7-((thiophen-2-ylmethyl)amino)thieno[3,2-b]pyridin-3-yl)pent-4-yne-1,2-diol;
2-((1S,6S)-6-aminocyclohex-3-en-1-yl)-3-bromo-5-chloro-N-(thiophen-3-ylmethyl)thieno[3,2-b]pyridin-7-amine;
2-((3R,4S)-4-aminotetrahydro-2H-pyran-3-yl)-N-benzyl-3,5-dichlorothieno[3,2-b]pyridin-7-amine;
6-((1S,6S)-6-aminocyclohex-3-en-1-yl)-N-(but-2-yn-1-yl)-2-chloro-7-iodothieno[3,2-d]pyrimidin-4-amine;
2-((1R,6R)-6-aminocyclohex-3-en-1-yl)-5-chloro-3-methyl-N-(thiophen-2-ylmethyl)thieno[3,2-b]pyridin-7-amine;
(3S,4S)-4-amino-3-(3,5-dichloro-7-((furan-2-ylmethyl)amino)thieno[3,2-b]pyridin-2-yl)cyclohexan-1-one;
2-((1S,6S)-6-aminocyclohex-3-en-1-yl)-5-chloro-3-methyl-N-(thiophen-2-ylmethyl)thieno[3,2-b]pyridin-7-amine;
6-((1S,6S)-6-aminocyclohex-3-en-1-yl)-2-chloro-7-methyl-N-(thiazol-2-ylmethyl)thieno[3,2-d]pyrimidin-4-amine;
2-((2R,3R)-3-aminotetrahydro-2H-pyran-2-yl)-3-bromo-5-chloro-1-(difluoromethyl)-N-(thiophen-2-ylmethyl)-1H-pyrrolo[3,2-b]pyridin-7-amine;
2-((2S,3S)-3-aminotetrahydro-2H-pyran-2-yl)-3-bromo-5-chloro-1-(difluoromethyl)-N-(thiophen-2-ylmethyl)-11H-pyrrolo[3,2-b]pyridin-7-amine;
6-((2S,3S)-3-aminotetrahydro-2H-pyran-2-yl)-2-chloro-5-(difluoromethyl)-N-(furan-2-ylmethyl)-7-iodo-5H-pyrrolo[3,2-d]pyrimidin-4-amine;
2-((1R,2R)-2-aminocyclohexyl)-N-benzyl-5-chloro-3-(prop-1-yn-1-yl)thieno[3,2-b]pyridin-7-amine;
2-((1S,2S)-2-aminocyclohexyl)-N-benzyl-5-chloro-3-(prop-1-yn-1-yl)thieno[3,2-b]pyridin-7-amine;
2-((3R,4S)-3-aminotetrahydro-2H-pyran-4-yl)-5-chloro-3-(prop-1-yn-1-yl)-N-(thiophen-2-ylmethyl)thieno[3,2-b]pyridin-7-amine;
2-((3S,4R)-3-aminotetrahydro-2H-pyran-4-yl)-5-chloro-3-(prop-1-yn-1-yl)-N-(thiophen-2-ylmethyl)thieno[3,2-b]pyridin-7-amine;
(1R,2R,3S)-2-amino-3-(3-bromo-15-chloro-7-((thiophen-2-ylmethyl)amino)thieno[3,2-b]pyridin-2-yl)cyclohexan-1-ol;
(1S,2S,3R)-2-amino-3-(3-bromo-5-chloro-7-((thiophen-2-ylmethyl)amino)thieno[3,2-b]pyridin-2-yl)cyclohexan-1-ol;
6-((1S,6S)-6-aminocyclohex-3-en-1-yl)-N-benzyl-2-chloro-7-iodothieno[3,2-d]pyrimidin-4-amine;
2-((1S,6S)-6-aminocyclohex-3-en-1-yl)-3-bromo-N-(but-2-yn-1-yl)-5-chlorothieno[3,2-b]pyridin-7-amine;
2-((1S,2S)-2-aminocyclohexyl)-5-chloro-N-(furan-2-ylmethyl)-3-methylthieno[3,2-b]pyridin-7-amine;
2-((3R,4S)-4-aminotetrahydro-2H-pyran-3-yl)-5-chloro-3-methyl-N-(thiophen-2-ylmethyl)thieno[3,2-b]pyridin-7-amine;
2-((1S,6S)-6-amino-3,4-dimethylcyclohex-3-en-1-yl)-3,5-dichloro-N-(furan-2-ylmethyl)thieno[3,2-b]pyridin-7-amine;
6-((1S,6S)-6-aminocyclohex-3-en-1-yl)-2-chloro-7-methyl-N-(pyridin-4-ylmethyl)thieno[3,2-d]pyrimidin-4-amine;
6-(2-((1S,6S)-6-aminocyclohex-3-en-1-yl)-5-chloro-7-((thiophen-2-ylmethyl)amino)thieno[3,2-b]pyridin-3-yl)hex-5-yn-1-ol;
6-((1S,6S)-6-aminocyclohex-3-en-1-yl)-2,7-dichloro-N-((3-fluoropyridin-4-yl)methyl)thieno[3,2-d]pyrimidin-4-amine;
2-(1S,2S)-2-aminocyclohexyl)-N-benzyl-3,5-dichlorothieno[3,2-b]pyridin-7-amine;
2-((3R,4S)-3-aminotetrahydro-2H-pyran-4-yl)-3-bromo-5-chloro-N-(furan-2-ylmethyl)thieno[3,2-b]pyridin-7-amine;
2-((1S,6S)-6-aminocyclohex-3-en-1-yl)-3,5-dichloro-N-((3-fluoropyridin-4-yl)methyl)thieno[3,2-b]pyridin-7-amine;
6-((1R,6R)-6-aminocyclohex-3-en-1-yl)-2,7-dichloro-N-(thiophen-2-ylmethyl)thieno[3,2-d]pyrimidin-4-amine;
6-((1S,6S)-6-aminocyclohex-3-en-1-yl)-2,7-dichloro-N-(thiophen-2-ylmethyl)thieno[3,2-d]pyrimidin-4-amine;
6-((1S,6S)-6-aminocyclohex-3-en-1-yl)-N-benzyl-7-bromo-2-chlorothieno[3,2-d]pyrimidin-4-amine;
2-((1S,6S)-6-aminocyclohex-3-en-1-yl)-3-chloro-7-((furan-2-ylmethyl)amino)thieno[3,2-b]pyridine-5-carbonitrile;
6-((2R,3R)-3-aminotetrahydro-2H-pyran-2-yl)-2-chloro-N-(furan-2-ylmethyl)-7-iodothieno[3,2-d]pyrimidin-4-amine;
6-((2S,3S)-3-aminotetrahydro-2H-pyran-2-yl)-2-chloro-N-(furan-2-ylmethyl)-7-iodothieno[3,2-d]pyrimidin-4-amine;
6-((2S,3S)-3-aminotetrahydro-2H-pyran-2-yl)-2,7-dichloro-5-(difluoromethyl)-N-(furan-2-ylmethyl)-5H-pyrrolo[3,2-d]pyrimidin-4-amine;
6-((3R,4S)-3-aminotetrahydro-2H-pyran-4-yl)-2-chloro-N-(furan-2-ylmethyl)-7-iodothieno[3,2-d]pyrimidin-4-amine;
6-((3S,4R)-3-aminotetrahydro-2H-pyran-4-yl)-2-chloro-N-(furan-2-ylmethyl)-7-iodothieno[3,2-d]pyrimidin-4-amine;
2-((1S,6S)-6-aminocyclohex-3-en-1-yl)-3-methyl-7-((thiophen-2-ylmethyl)amino)thieno[3,2-b]pyridine-5-carbonitrile;
2-((1S,6S)-6-aminocyclohex-3-en-1-yl)-5-chloro-3-methyl-N-(thiophen-3-ylmethyl)thieno[3,2-b]pyridin-7-amine;
6-((2S,3S)-3-aminotetrahydro-2H-pyran-2-yl)-7-bromo-2-chloro-5-(difluoromethyl)-N-(thiophen-2-ylmethyl)-5H-pyrrolo[3,2-d]pyrimidin-4-amine;
2-((1S,6S)-6-amino-3,4-dimethylcyclohex-3-en-1-yl)-3-bromo-N-(but-2-yn-1-yl)-5-chlorothieno[3,2-b]pyridin-7-amine;
2-((1S,6S)-6-aminocyclohex-3-en-1-yl)-N-(but-2-yn-1-yl)-3,5-dichlorothieno[3,2-b]pyridin-7-amine;
2-((2S,3S)-3-aminotetrahydro-2H-pyran-2-yl)-3-bromo-5-chloro-N-(pyridin-4-ylmethyl)thieno[3,2-b]pyridin-7-amine;
3-(2-((2R,3R)-3-aminotetrahydro-2H-pyran-2-yl)-5-chloro-7-((thiophen-2-ylmethyl)amino)thieno[3,2-b]pyridin-3-yl)prop-2-yn-1-ol;
3-(2-((2S,3S)-3-aminotetrahydro-2H-pyran-2-yl)-5-chloro-7-((thiophen-2-ylmethyl)amino)thieno[3,2-b]pyridin-3-yl)prop-2-yn-1-ol;
6-((1S,6S)-6-aminocyclohex-3-en-1-yl)-2-chloro-N-((3-fluoropyridin-4-yl)methyl)-7-methylthieno[3,2-d]pyrimidin-4-amine;
6-((2S,3S)-3-aminotetrahydro-2H-pyran-2-yl)-2-chloro-5-(difluoromethyl)-7-iodo-N-(thiophen-2-ylmethyl)-5H-pyrrolo[3,2-d]pyrimidin-4-amine;
3-(6-((2R,3R)-3-aminotetrahydro-2H-pyran-2-yl)-2-chloro-4-((furan-2-ylmethyl)amino)thieno[3,2-d]pyrimidin-7-yl)prop-2-yn-1-ol;
3-(6-((2S,3S)-3-aminotetrahydro-2H-pyran-2-yl)-2-chloro-4-((furan-2-ylmethyl)amino)thieno[3,2-d]pyrimidin-7-yl)prop-2-yn-1-ol;
2-((1S,6S)-6-aminocyclohex-3-en-1-yl)-3-bromo-5-chloro-N-(thiophen-2-ylmethyl)thieno[3,2-b]pyridin-7-amine;
2-((1R,6R)-6-aminocyclohex-3-en-1-yl)-3-bromo-5-chloro-N-(thiophen-2-ylmethyl)thieno[3,2-b]pyridin-7-amine;
2-((1S,2S)-2-aminocyclohexyl)-5-chloro-3-((difluoromethoxy)methyl)-N-(thiophen-2-ylmethyl)thieno[3,2-b]pyridin-7-amine;
2-((1R,2R)-2-aminocyclohexyl)-5-chloro-3-((difluoromethoxy)methyl)-N-(thiophen-2-ylmethyl)thieno[3,2-b]pyridin-7-amine;
2-((1S,6S)-6-aminocyclohex-3-en-1-yl)-N-benzyl-5-chloro-3-methylthieno[3,2-b]pyridin-7-amine;
2-((2R,3R)-3-amino-5-methylenetetrahydro-2H-pyran-2-yl)-3-bromo-5-chloro-N-(thiophen-2-ylmethyl)thieno[3,2-b]pyridin-7-amine;
2-((2S,3S)-3-amino-5-methylenetetrahydro-2H-pyran-2-yl)-3-bromo-5-chloro-N-(thiophen-2-ylmethyl)thieno[3,2-b]pyridin-7-amine;
2-((3R,4S)-4-aminotetrahydro-2-pyran-3-yl)-5-chloro-N-(furan-2-ylmethyl)-3-methylthieno[3,2-b]pyridin-7-amine;
2-((1S,6S)-6-aminocyclohex-3-en-1-yl-2,2,3,4,5,5-d6)-5-chloro-3-methyl-N-(thiophen-2-ylmethyl)thieno[3,2-b]pyridin-7-amine;
2-((2S,3S)-3-aminotetrahydro-2H-pyran-2-yl)-3,5-dichloro-1-(difluoromethyl)-N-(thiophen-2-ylmethyl)-1H-pyrrolo[3,2-b]pyridin-7-amine;
6-((1S,6S)-6-aminocyclohex-3-en-1-yl)-N-benzyl-2,7-dichlorothieno[3,2-d]pyrimidin-4-amine;
5-(5-chloro-2-((1S,2S)-2-(methylamino)cyclohexyl)-7-((thiophen-2-ylmethyl)amino)thieno[3,2-b]pyridin-3-yl)pent-4-yn-1-ol;
2-((3R,4S)-3-aminotetrahydro-2H-pyran-4-yl)-5-chloro-3-iodo-N-(thiazol-2-ylmethyl)thieno[3,2-b]pyridin-7-amine;
2-((2R,3R)-3-aminotetrahydro-2H-pyran-2-yl)-5-chloro-3-iodo-N-(thiophen-2-ylmethyl)thieno[3,2-b]pyridin-7-amine;
2-((2R,3R)-3-aminotetrahydro-2H-pyran-2-yl)-5-chloro-3-iodo-N-(thiophen-2-ylmethyl)thieno[3,2-b]pyridin-7-amine;
2-((1S,6S)-6-aminocyclohex-3-en-1-yl)-5-chloro-N-(thiophen-2-ylmethyl)-3-vinylthieno[3,2-b]pyridin-7-amine;
6-((1S,6S)-6-aminocyclohex-3-en-1-yl)-2-chloro-7-methyl-N-(thiophen-2-ylmethyl)thieno[3,2-d]pyrimidin-4-amine;
2-((1R,6S)-6-amino-2,2-difluorocyclohexyl)-3-bromo-5-chloro-N-(thiophen-2-ylmethyl)thieno[3,2-b]pyridin-7-amine;
2-((1S,6R)-6-amino-2,2-difluorocyclohexyl)-3-bromo-5-chloro-N-(thiophen-2-ylmethyl)thieno[3,2-b]pyridin-7-amine;
2-((2S,3S)-3-aminotetrahydro-2H-pyran-2-yl)-3-bromo-5-chloro-1-(difluoromethyl)-N-(furan-2-ylmethyl)-1H-pyrrolo[3,2-b]pyridin-7-amine;
6-((1S,3S)-3-aminotetrahydro-2H-pyran-2-yl)-2,7-dichloro-5-(difluoromethyl)-N-(furan-2-ylmethyl)-5H-pyrrolo[3,2-d]pyrimidin-4-amine;
2-((3R,4S)-3-aminotetrahydro-2H-pyran-4-yl)-3,5-dichloro-N-(thiophen-2-ylmethyl)thieno[3,2-b]pyridin-7-amine;
2-((3S,4R)-3-aminotetrahydro-2H-pyran-4-yl)-3,5-dichloro-N-(thiophen-2-ylmethyl)thieno[3,2-b]pyridin-7-amine;
2-((2S,3S)-3-aminotetrahydro-2H-pyran-2-yl)-3,5-dichloro-1-(difluoromethyl)-N-(furan-2-ylmethyl)-1H-pyrrolo[3,2-b]pyridin-7-amine;
(4S,5S)-5-amino-4-(5-chloro-3-methyl-7-((thiophen-2-ylmethyl)amino)thieno[3,2-b]pyridin-2-yl)cyclohex-1-ene-1-carboxamide;
2-((3R,4S)-3-aminotetrahydro-2H-pyran-4-yl)-5-chloro-3-iodo-N-(thiophen-2-ylmethyl)thieno[3,2-b]pyridin-7-amine;
2-4(3S,4R)-3-aminotetrahydro-2H-pyran-4-yl)-5-chloro-3-iodo-N-(thiophen-2-ylmethyl)thieno[3,2-b]pyridin-7-amine;
6-((1S,6S)-6-aminocyclohex-3-en-1-yl)-2-chloro-5-(difluoromethyl)-N-(furan-2-ylmethyl)-7-iodo-5H-pyrrolo[3,2-d]pyrimidin-4-amine;
ethyl (4S,5S)-5-amino-4-(5-chloro-3-methyl-7-((thiophen-2-ylmethyl)amino)thieno[3,2-b]pyridin-2-yl)cyclohex-1-ene-1-carboxylate;
2-((1R,6R)-6-aminocyclohex-3-en-1-yl-2,2,3,4,5,5-d6)-N-benzyl-5-chloro-3-methylthieno[3,2-b]pyridin-7-amine;
2-((1S,6S)-6-aminocyclohex-3-en-1-yl-2,2,3,4,5,5-d6)-N-benzyl-5-chloro-3-methylthieno[3,2-b]pyridin-7-amine;
2-((2R)-2-aminocyclohexyl)-5-chloro-3-(difluoromethyl)-N-(thiophen-2-ylmethyl)thieno[3,2-b]pyridin-7-amine;
2-((2S)-2-aminocyclohexyl)-5-chloro-3-(difluoromethyl)-N-(thiophen-2-ylmethyl)thieno[3,2-b]pyridin-7-amine;
2-((1S,2S)-2-aminocyclohexyl)-N-benzyl-5-chloro-3-methylthieno[3,2-b]pyridin-7-amine;
2-((2S,3S)-3-aminotetrahydro-2H-pyran-2-yl)-3-bromo-5-chloro-1-(difluoromethyl)-N-(thiophen-2-ylmethyl)-1-pyrrolo[3,2-b]pyridin-7-amine;
6-((1S,6S)-6-aminocyclohex-3-en-1-yl)-4-((furan-2-ylmethyl)amino)-7-methylthieno[3,2-d]pyrimidine-2-carbonitrile;
2-((1S,2S)-2-aminocyclohexyl)-N-(but-2-yn-1-yl)-5-chloro-3-iodothieno[3,2-b]pyridin-7-amine;
2-((2R,3R,5R)-3-amino-5-methyltetrahydro-2H-pyran-2-yl)-3-bromo-5-chloro-N-(thiophen-2-ylmethyl)thieno[3,2-b]pyridin-7-amine;
2-((2S,3S,5S)-3-amino-5-methyltetrahydro-2H-pyran-2-yl)-3-bromo-5-chloro-N-(thiophen-2-ylmethyl)thieno[3,2-b]pyridin-7-amine;
2-((3R,4S)-3-aminotetrahydro-2H-pyran-4-yl)-3-bromo-5-chloro-N-(thiazol-2-ylmethyl)thieno[3,2-b]pyridin-7-amine;
6-((1S,2S)-2-aminocyclohexyl)-N-(but-2-yn-1-yl)-2-chloro-7-iodothieno[3,2-d]pyrimidin-4-amine;
2-((1S,6S)-6-aminocyclohex-3-en-1-yl)-3-bromo-5-chloro-N-(2-fluorobenzyl)thieno[3,2-b]pyridin-7-amine;
5-(5-chloro-2-((1R,6R)-6-(methylamino)cyclohex-3-en-1-yl)-7-((thiophen-2-ylmethyl)amino)thieno[3,2-b]pyridin-3-yl)pent-4-yn-1-ol;
5-(5-chloro-2-((1S,6S)-6-(methylamino)cyclohex-3-en-1-yl)-7-((thiophen-2-ylmethyl)amino)thieno[3,2-b]pyridin-3-yl)pent-4-yn-1-ol;
2-((1S,6S)-6-aminocyclohex-3-en-1-yl)-7-((furan-2-ylmethyl)amino)-3-methylthieno[3,2-b]pyridine-5-carbonitrile;
2-((1R,3R,4R,6S)-4-aminobicyclo[4.1.0]heptan-3-yl)-3-bromo-5-chloro-N-(thiophen-2-ylmethyl)thieno[3,2-b]pyridin-7-amine;
2-((1S,3S,4S,6R)-4-aminobicyclo[4.1.0]heptan-3-yl)-3-bromo-5-chloro-N-(thiophen-2-ylmethyl)thieno[3,2-b]pyridin-7-amine;
2-((1R,6S)-6-amino-2,2-difluorocyclohexyl)-3,5-dichloro-N-(thiophen-2-ylmethyl)thieno[3,2-b]pyridin-7-amine;
2-((1S,6S)-6-aminocyclohex-3-en-1-yl-2,2,3,4,5,5-d6)-N-benzyl-5-chloro-3-methylthieno[3,2-b]pyridin-7-amine;
5-(2-chloro-6-((1R,6R)-6-(methylamino)cyclohex-3-en-1-yl)-4-((thiophen-2-ylmethyl)amino)thieno[3,2-d]pyrimidin-7-yl)pent-4-yn-1-ol;
5-(2-chloro-6-((1S,6S)-6-(methylamino)cyclohex-3-en-1-yl)-4-((thiophen-2-ylmethyl)amino)thieno[3,2-d]pyrimidin-7-yl)pent-4-yn-1-ol;
2-((1R,5S,6R)-6-amino-5-methylcyclohex-3-en-1-yl)-3-bromo-5-chloro-N-(thiophen-2-ylmethyl)thieno[3,2-b]pyridin-7-amine;
2-((1S,5R,6S)-6-amino-5-methylcyclohex-3-en-1-yl)-3-bromo-5-chloro-N-(thiophen-2-ylmethyl)thieno[3,2-b]pyridin-7-amine;
2-((1R,2R)-2-aminocyclohexyl)-N-benzyl-5-chloro-3-iodothieno[3,2-b]pyridin-7-amine;
2-((1S,2S)-2-aminocyclohexyl)-N-benzyl-5-chloro-3-iodothieno[3,2-b]pyridin-7-amine;
2-((1R,2R)-2-aminocyclohexyl)-3,5-dichloro-N-(furan-2-ylmethyl)thieno[3,2-b]pyridin-7-amine;
6-((1R,6R)-6-aminocyclohex-3-en-1-yl-2,2,3,4,5,5-d6)-2-chloro-N-(furan-2-ylmethyl)-7-methylthieno[3,2-d]pyrimidin-4-amine;
6-((1S,6S)-6-aminocyclohex-3-en-1-yl-2,2,3,4,5,5-d6)-2-chloro-N-(furan-2-ylmethyl)-7-methylthieno[3,2-d]pyrimidin-4-amine;
(1R,2R,3S)-2-amino-3-(3,5-dichloro-7-((thiophen-2-ylmethyl)amino)thieno[3,2-b]pyridin-2-yl)cyclohexan-1-ol;
2-((1R,6R)-6-aminocyclohex-3-en-1-yl)-3,5-dichloro-N-(furan-2-ylmethyl)thieno[3,2-b]pyridin-7-amine;
2-((1R,6R)-6-aminocyclohex-3-en-1-yl)-3-bromo-N-((E)-but-2-en-1-yl)-5-chlorothieno[3,2-b]pyridin-7-amine;
2-((1S,6S)-6-aminocyclohex-3-en-1-yl)-3-bromo-N-((E)-but-2-en-1-yl)-5-chlorothieno[3,2-b]pyridin-7-amine;
2-((1S,2S)-2-aminocyclohexyl)-3-bromo-N-(but-2-yn-1-yl)-5-chlorothieno[3,2-b]pyridin-7-amine;
2-((1S,6S)-6-aminocyclohex-3-en-1-yl)-N-((E)-but-2-en-1-yl)-3,5-dichlorothieno[3,2-b]pyridin-7-amine;
4-(5-chloro-2-((1S,6S)-6-(methylamino)cyclohex-3-en-1-yl)-7-((thiophen-2-ylmethyl)amino)thieno[3,2-b]pyridin-3-yl)but-3-yn-1-ol;
6-((1S,2S)-2-aminocyclohexyl)-2-chloro-5-(difluoromethyl)-N-(furan-2-ylmethyl)-7-iodo-5H-pyrrolo[3,2-d]pyrimidin-4-amine;
6-((2S,3S)-3-aminotetrahydro-2H-pyran-2-yl)-2,7-dichloro-5-(difluoromethyl)-N-(thiophen-2-ylmethyl)-5H-pyrrolo[3,2-d]pyrimidin-4-amine;
2-((1S,6S)-6-aminocyclohex-3-en-1-yl)-N-(but-2-yn-1-yl)-5-chloro-3-methylthieno[3,2-b]pyridin-7-amine;
2-((1S,6S)-6-aminocyclohex-3-en-1-yl)-5-chloro-3-(pyridin-3-ylethynyl)-N-(thiophen-2-ylmethyl)thieno[3,2-b]pyridin-7-amine;
2-((1S,2S)-2-aminocyclohexyl)-N-(but-2-yn-1-yl)-3,5-dichlorothieno[3,2′-b]pyridin-7-amine;
5-(5-chloro-2-((2R,3R)-3-(methylamino)tetrahydro-2H-pyran-2-yl)-7-((thiophen-2-ylmethyl)amino)thieno[3,2-b]pyridin-3-yl)pent-4-yn-1-ol;
5-(5-chloro-2-((2S,3S)-3-(methylamino)tetrahydro-2H-pyran-2-yl)-7-((thiophen-2-ylmethyl)amino)thieno[3,2-b]pyridin-3-yl)pent-4-yn-1-ol;
2-((1R,6R)-6-aminocyclohex-3-en-1-yl)-3-bromo-5-chloro-N-(furan-2-ylmethyl)thieno[3,2-b]pyridin-7-amine;
2-((1S,2S)-2-aminocyclohexyl)-5-chloro-3-ethynyl-N-(2-fluorobenzyl)thieno[3,2-b]pyridin-7-amine;
(4S,5S)-5-amino-4-(5-chloro-3-methyl-7-((thiophen-2-ylmethyl)amino)thieno[3,2-b]pyridin-2-yl)-N,N-dimethylcyclohex-1-ene-1-carboxamide;
((4S,5S)-5-amino-4-(5-chloro-3-methyl-7-((thiophen-2-ylmethyl)amino)thieno[3,2-b]pyridin-2-yl)cyclohex-1-en-1-yl)(morpholino)methanone;
2-((3R,4S)-3-aminotetrahydro-2H-pyran-4-yl)-3,5-dichloro-N-(furan-2-ylmethyl)thieno[3,2-b]pyridin-7-amine;
2-((3S,4R)-3-aminotetrahydro-2H-pyran-4-yl)-3,5-dichloro-N-(furan-2-ylmethyl)thieno[3,2-b]pyridin-7-amine;
2-((3R,4S)-3-aminotetrahydro-2H-pyran-4-yl)-3,5-dichloro-N-(thiophen-2-ylmethyl)thieno[3,2-b]pyridin-7-amine;
2-((3S,4R)-3-aminotetrahydro-2H-pyran-4-yl)-3,5-dichloro-N-(thiophen-2-ylmethyl)thieno[3,2-b]pyridin-7-amine;
2-((3R,4S)-3-aminotetrahydro-2H-pyran-4-yl)-3-bromo-5-chloro-N-(furan-2-ylmethyl)thieno[3,2-b]pyridin-7-amine;
2-((3S,4R)-3-aminotetrahydro-2H-pyran-4-yl)-3-bromo-5-chloro-N-(furan-2-ylmethyl)thieno[3,2-b]pyridin-7-amine;
6-((3R,4S)-3-aminotetrahydro-2H-pyran-4-yl)-2-chloro-N-(furan-2-ylmethyl)-7-((trimethylsilyl)ethynyl)thieno[3,2-d]pyrimidin-4-amine;
6-((3S,4R)-3-aminotetrahydro-2H-pyran-4-yl)-2-chloro-N-(furan-2-ylmethyl)-7-((trimethylsilyl)ethynyl)thieno[3,2-d]pyrimidin-4-amine;
6-((1R,2R)-2-aminocyclohexyl)-2-chloro-5-(difluoromethyl)-N-(furan-2-ylmethyl)-7-(prop-1-yn-1-yl)-5H-pyrrolo[3,2-d]pyrimidin-4-amine;
2-((3R,4S)-3-aminotetrahydro-2H-pyran-4-yl)-3,5-dichloro-N-(furan-2-ylmethyl)thieno[3,2-b]pyridin-7-amine;
2-((3S,4R)-3-aminotetrahydro-2H-pyran-4-yl)-3,5-dichloro-N-(furan-2-ylmethyl)thieno[3,2-b]pyridin-7-amine;
7-(2-((1S,6S)-6-aminocyclohex-3-en-1-yl)-5-chloro-7-((thiophen-2-ylmethyl)amino)thieno[3,2-b]pyridin-3-yl)hept-6-yn-1-ol;
2-((3R,4S)-4-aminotetrahydro-2H-1-pyran-3-yl)-N-benzyl-5-chloro-3-methylthieno[3,2-b]pyridin-7-amine;
2-((1S,6S)-6-aminocyclohex-3-en-1-yl-2,2,3,4,5,5-d6)-5-chloro-3-methyl-N-(phenylmethyl-d2)thieno[3,2-b]pyridin-7-amine;
6-((1R,6R)-6-aminocyclohex-3-en-1-yl)-2-chloro-N-(furan-2-ylmethyl)-7-iodothieno[3,2-d]pyrimidin-4-amine;
2-((1R,6R)-6-aminocyclohex-3-en-1-yl)-5-chloro-3-(propa-1,2-dien-1-yl)-N-(thiophen-2-ylmethyl)thieno[3,2-b]pyridin-7-amine;
2-((1S,6S)-6-aminocyclohex-3-en-1-yl)-5-chloro-3-(propa-1,2-dien-1-yl)-N-(thiophen-2-ylmethyl)thieno[3,2-b]pyridin-7-amine;
2-((1S,6S)-6-aminocyclohex-3-en-1-yl)-5-chloro-1-(difluoromethyl)-3-iodo-N-(thiophen-2-ylmethyl)-1H-pyrrolo[3,2-b]pyridin-7-amine;
6-((1S,6S)-6-aminocyclohex-3-en-1-yl)-N-benzyl-2-chloro-7-methylthieno[3,2-d]pyrimidin-4-amine;
2-((1R,6R)-6-aminocyclohex-3-en-1-yl)-5-chloro-N-(furan-2-ylmethyl)-3-iodothieno[3,2-b]pyridin-7-amine;
2-((3R,4S)-3-aminotetrahydro-2H-pyran-4-yl)-N-benzyl-3-bromo-5-chlorothieno[3,2-b]pyridin-7-amine;
2-(3S,4R)-3-aminotetrahydro-2H-pyran-4-yl)-N-benzyl-3-bromo-5-chlorothieno[3,2-b]pyridin-7-amine;
(1R,5R,6S)-5-amino-6-(3-bromo-5-chloro-7-(thiophen-2-ylmethyl)amino)thieno[3,2-b]pyridin-2-yl)cyclohex-2-en-1-ol;
(0S,5S,6R)-5-amino-6-(3-bromo-5-chloro-7-((thiophen-2-ylmethyl)amino)thieno[3,2-b]pyridin-2-yl)cyclohex-2-en-1-ol;
6-((3R,4S)-3-aminotetrahydro-2H-pyran-4-yl)-2-chloro-7-ethynyl-N-(furan-2-ylmethyl)thieno[3,2-d]pyrimidin-4-amine;
6-((3S,4R)-3-aminotetrahydro-2H-pyran-4-yl)-2-chloro-7-ethynyl-N-(furan-2-ylmethyl)thieno[3,2-d]pyrimidin-4-amine;
6-((1S,2S)-2-aminocyclohexyl)-2-chloro-5-(difluoromethyl)-N-(furan-2-ylmethyl)-7-(prop-1-yn-1-yl)-5H-pyrrolo[3,2-d]pyrimidin-4-amine;
2-((1R,6R)-6-aminocyclohex-3-en-1-yl)-3-bromo-5-chloro-N-(cyclopent-1-en-1-ylmethyl)thieno[3,2-b]pyridin-7-amine;
2-((1S,6S)-6-aminocyclohex-3-en-1-yl)-3-bromo-5-chloro-N-(cyclopent-1-en-1-ylmethyl)thieno[3,2-b]pyridin-7-amine;
2-((1R,2R)-2-amino-5,5-difluorocyclohexyl)-5-chloro-3-methyl-N-(thiophen-2-ylmethyl)thieno[3,2-b]pyridin-7-amine;
2-((1S,2S)-2-amino-5,5-difluorocyclohexyl)-5-chloro-3-methyl-N-(thiophen-2-ylmethyl)thieno[3,2-b]pyridin-7-amine;
6-((1S,2S)-2-aminocyclohexyl)-2,7-dichloro-5-(difluoromethyl)-N-(furan-2-ylmethyl)-5H-pyrrolo[3,2-d]pyrimidin-4-amine;
6-((1R,2R)-2-aminocyclohexyl)-2,7-dichloro-5-(difluoromethyl)-N-(furan-2-ylmethyl)-5H-pyrrolo[3,2-d]pyrimidin-4-amine;
2-((1R,2R,6S)-2-amino-6-fluorocyclohexyl)-3-bromo-5-chloro-N-(thiophen-2-ylmethyl)thieno[3,2-b]pyridin-7-amine;
2-((1S,2S,6R)-2-amino-6-fluorocyclohexyl)-3-bromo-5-chloro-N-(thiophen-2-ylmethyl)thieno[3,2-b]pyridin-7-amine;
5-(2-((1 S,6S)-6-aminocyclohex-3-en-1-yl)-5-chloro-7-((furan-2-ylmethyl)(methyl)amino)thieno[3,2-b]pyridin-3-yl)pent-4-yn-1-ol;
2-((3R,4S)-3-aminotetrahydro-2H-pyran-4-yl)-3,5-dichloro-N-(thiazol-2-ylmethyl)thieno[3,2-b]pyridin-7-amine;
2-((1R,6S)-6-amino-2,2-difluorocyclohexyl)-5-chloro-3-methyl-N-(thiophen-2-ylmethyl)thieno[3,2-b]pyridin-7-amine;
6-((3R,4S)-3-aminotetrahydro-2H-pyran-4-yl)-7-bromo-2-chloro-N-(furan-2-ylmethyl)thieno[3,2-d]pyrimidin-4-amine;
6-((3S,4R)-3-aminotetrahydro-2H-pyran-4-yl)-7-bromo-2-chloro-N-(furan-2-ylmethyl)thieno[3,2-d]pyrimidin-4-amine;
6-((1S,6S)-6-aminocyclohex-3-en-1-yl)-2,7-dichloro-N-(thiazol-2-ylmethyl)thieno[3,2-d]pyrimidin-4-amine;
2-((1S,6S)-6-aminocyclohex-3-en-1-yl)-5-chloro-N-(2-fluorobenzyl)-3-methylthieno[3,2-b]pyridin-7-amine;
2-((1R,2S,3S)-2-amino-3-fluorocyclohexyl)-5-chloro-3-methyl-N-(thiophen-2-ylmethyl)thieno[3,2-b]pyridin-7-amine;
2-((1S,2R,3R)-2-amino-3-fluorocyclohexyl)-5-chloro-3-methyl-N-(thiophen-2-ylmethyl)thieno[3,2-b]pyridin-7-amine;
2-((2S,3S)-3-aminotetrahydro-2H-pyran-2-yl)-3-bromo-N-(but-2-yn-1-yl)-5-chlorothieno[3,2-b]pyridin-7-amine;
2-((1R,2R)-2-aminocyclohexyl)-N-benzyl-5-chloro-3-ethynylthieno[3,2-b]pyridin-7-amine;
2-((1R,6R)-6-aminocyclohex-3-en-1-yl)-N-benzyl-5-chloro-3-iodothieno[3,2-b]pyridin-7-amine;
6-((2S,3S)-3-aminotetrahydro-2H-pyran-2-yl)-2,7-dichloro-5-(difluoromethyl)-N-(thiophen-2-ylmethyl)-5H-pyrrolo[3,2-d]pyrimidin-4-amine;
6-((2R,3R)-3-aminotetrahydro-2H-pyran-2-yl)-2,7-dichloro-5-(difluoromethyl)-N-(thiophen-2-ylmethyl)-5H-pyrrolo[3,2-d]pyrimidin-4-amine;
6-((3R,4S)-3-aminotetrahydro-2H-pyran-4-yl)-7-bromo-2-chloro-N-(thiophen-2-ylmethyl)thieno[3,2-d]pyrimidin-4-amine;
6-((3S,4R)-3-aminotetrahydro-2H-pyran-4-yl)-7-bromo-2-chloro-N-(thiophen-2-ylmethyl)thieno[3,2-d]pyrimidin-4-amine;
6-((1S,6S)-6-aminocyclohex-3-en-1-yl)-N-benzyl-2-chloro-5-(difluoromethyl)-7-iodo-5H-pyrrolo[3,2-d]pyrimidin-4-amine;
5-(2-((1S,6S)-6-aminocyclohex-3-en-1-yl)-5-chloro-7-(methyl(thiophen-2-ylmethyl)amino)thieno[3,2-b]pyridin-3-yl)pent-4-yn-1-ol;
2-4(3R,4S)-3-aminotetrahydro-2H-pyran-4-yl)-5-chloro-N-(thiophen-2-ylmethyl)-3-vinylthieno[3,2-b]pyridin-7-amine;
2-((3S,4R)-3-aminotetrahydro-2H-pyran-4-yl)-5-chloro-N-(thiophen-2-ylmethyl)-3-vinylthieno[3,2-b]pyridin-7-amine;
2-((3R,4S)-3-aminotetrahydro-2H-pyran-4-yl)-3-bromo-5-chloro-1-methyl-N-(thiophen-2-ylmethyl)-1H-pyrrolo[3,2-b]pyridin-7-amine;
2-((1S,6S)-6-aminocyclohex-3-en-1-yl)-N-benzyl-5-chloro-1-(difluoromethyl)-3-iodo-1H-pyrrolo[3,2-b]pyridin-7-amine;
(4S,5S)-5-amino-4-(5-chloro-3-methyl-7-((thiophen-2-ylmethyl)amino)thieno[3,2-b]pyridin-2-yl)-N-phenylcyclohex-1-ene-1-carboxamide;
6-((3R,4S)-3-aminotetrahydro-2H-pyran-4-yl)-2,7-dichloro-N-(furan-2-ylmethyl)thieno[3,2-d]pyrimidin-4-amine;
6-((3S,4R)-3-aminotetrahydro-2H-pyran-4-yl)-2,7-dichloro-N-(furan-2-ylmethyl)thieno[3,2-d]pyrimidin-4-amine;
(1R,2S,3R)-3-amino-2-(3-bromo-5-chloro-7-((thiophen-2-ylmethyl)amino)thieno[3,2-b]pyridin-2-yl)cyclohexan-1-ol;
(1S,2R,3S)-3-amino-2-(3-bromo-5-chloro-7-((thiophen-2-ylmethyl)amino)thieno[3,2-b]pyridin-2-yl)cyclohexan-1-ol;
2-((1S,2S)-2-aminocyclohexyl)-5-chloro-N-(thiophen-2-ylmethyl)-3-(trifluoromethyl)thieno[3,2-b]pyridin-7-amine;
2-((1R,2R)-2-aminocyclohexyl)-5-chloro-N-(thiophen-2-ylmethyl)-3-(trifluoromethyl)thieno[3,2-b]pyridin-7-amine;
2-((1S,2S)-2-aminocyclohexyl)-5-chloro-3-ethynyl-N-(furan-2-ylmethyl)thieno[3,2-b]pyridin-7-amine;
6-((1R,6R)-6-aminocyclohex-3-en-1-yl)-2-chloro-5-(difluoromethyl)-N-(furan-2-ylmethyl)-7-methyl-5H-pyrrolo[3,2-d]pyrimidin-4-amine;
6-((1S,6S)-6-aminocyclohex-3-en-1-yl)-2-chloro-5-(difluoromethyl)-N-(furan-2-ylmethyl)-7-methyl-5H-pyrrolo[3,2-d]pyrimidin-4-amine;
2-((1R,6R)-6-amino-2,2-difluorocyclohexyl)-3-bromo-5-chloro-N-(thiophen-2-ylmethyl)thieno[3,2-b]pyridin-7-amine;
2-((1S,6S)-6-amino-2,2-difluorocyclohexyl)-3-bromo-5-chloro-N-(thiophen-2-ylmethyl)thieno[3,2-b]pyridin-7-amine;
(2-((1S,2S)-2-aminocyclohexyl)-5-chloro-7-((thiophen-2-ylmethyl)amino)thieno[3,2-b]pyridin-3-yl)methanol;
(2-((1R,2R)-2-amino cyclohexyl)-5-chloro-7-((thiophen-2-ylmethyl)amino)thieno[3,2-b]pyridin-3-yl)methanol 2-((1S,2S)-2-aminocyclohexyl)-3-bromo-5-chloro-N-(cyclopent-1-en-1-ylmethyl)thieno[3,2-b]pyridin-7-amine;
6-((1R,6R)-6-aminocyclohex-3-en-1-yl)-2-chloro-7-iodo-N-(thiophen-2-ylmethyl)thieno[3,2-d]pyrimidin-4-amine;
2-((1S,2S)-2-aminocyclohexyl)-N-(but-2-yn-1-yl)-5-chloro-3-methylthieno[3,2-b]pyridin-7-amine;
2-((3R,4S)-3-aminotetrahydro-2H-pyran-4-yl)-3-bromo-5-chloro-N-(pyridin-4-ylmethyl)thieno[3,2-b]pyridin-7-amine;
2-((1R,6R)-6-aminocyclohex-3-en-1-yl)-5-chloro-3-iodo-N-(thiophen-2-ylmethyl)thieno[3,2-b]pyridin-7-amine;
2-((1S,6S)-6-aminocyclohex-3-en-1-yl)-5-chloro-3-iodo-N-(thiophen-2-ylmethyl)thieno[3,2-b]pyridin-7-amine;
methyl (2-((1S,6S)-6-aminocyclohex-3-en-1-yl)-3-bromo-5-chlorothieno[3,2-b]pyridin-7-yl)glycinate;
2-((1R,6R)-6-aminocyclohex-3-en-1-yl)-5-chloro-3-methyl-N-(thiophen-2-ylmethyl)thieno[3,2-b]pyridin-7-amine;
2-((1S,6S)-6-aminocyclohex-3-en-1-yl)-5-chloro-3-methyl-N-(thiophen-2-ylmethyl)thieno[3,2-b]pyridin-7-amine;
2-((1R,2R)-2-aminocyclohexyl)-N-benzyl-3,5-dichlorothieno[3,2-b]pyridin-7-amine;
2-((1R,6R)-6-aminocyclohex-3-en-1-yl-2,2,3,4,5,5-d6)-3,5-dichloro-N-(furan-2-ylmethyl)thieno[3,2-b]pyridin-7-amine;
2-((1R,6S)-6-amino-2,2-difluorocyclohexyl)-3-bromo-N-(but-2-yn-1-yl)-5-chlorothieno[3,2-b]pyridin-7-amine;
2-((1S,6R)-6-amino-2,2-difluorocyclohexyl)-3-bromo-N-(but-2-yn-1-yl)-5-chlorothieno[3,2-b]pyridin-7-amine;
2-((1R,5S,6S)-6-amino-5-methoxycyclohex-3-en-1-yl)-3-bromo-5-chloro-N-(thiophen-2-ylmethyl)thieno[3,2-b]pyridin-7-amine;
2-((1S,5R,6R)-6-amino-5-methoxycyclohex-3-en-1-yl)-3-bromo-5-chloro-N-(thiophen-2-ylmethyl)thieno[3,2-b]pyridin-7-amine;
6-((1R,6R)-6-aminocyclohex-3-en-1-yl-2,2,3,4,5,5-d6)-7-bromo-2-chloro-N-(thiophen-2-ylmethyl)thieno[3,2-d]pyrimidin-4-amine;
2-((1R,6R)-6-aminocyclohex-3-en-1-yl)-3-bromo-5-chloro-N-(pyridin-4-ylmethyl)thieno[3,2-b]pyridin-7-amine;
(R,2S,3R)-2-amino-3-(5-chloro-3-methyl-7-((thiophen-2-ylmethyl)amino)thieno[3,2-b]pyridin-2-yl)cyclohexan-1-ol;
(1S,2R,3S)-2-amino-3-(5-chloro-3-methyl-7-((thiophen-2-ylmethyl)amino)thieno[3,2-b]pyridin-2-yl)cyclohexan-1-ol;
3-bromo-5-chloro-2-((1R,2R)-2-(methylamino)cyclohexyl)-N-(thiophen-2-ylmethyl)thieno[3,2-b]pyridin-7-amine;
3-bromo-5-chloro-2-((1S,2S)-2-(methylamino)cyclohexyl)-N-(thiophen-2-ylmethyl)thieno[3,2-b]pyridin-7-amine;
2-((1R,6R)-6-aminocyclohex-3-en-1-yl)-3,5-dichloro-N-((3-fluoropyridin-4-yl)methyl)thieno[3,2-b]pyridin-7-amine;
(1R,5S,6R)-5-amino-6-(3,5-dichloro-7-((thiophen-2-ylmethyl)amino)thieno[3,2-b]pyridin-2-yl)cyclohex-2-en-1-ol;
2-((1S,6S)-6-aminocyclohex-3-en-1-yl)-N-((E)-but-2-en-1-yl)-5-chloro-3-methylthieno[3,2-b]pyridin-7-amine;
6-((1R,6R)-6-aminocyclohex-3-en-1-yl)-2-chloro-5-(difluoromethyl)-N-(furan-2-ylmethyl)-7-iodo-5H-pyrrolo[3,2-d]pyrimidin-4-amine;
2-((1R,6R)-6-aminocyclohex-3-en-1-yl)-3,5-dichloro-N-(thiazol-2-ylmethyl)thieno[3,2-b]pyridin-7-amine;
2-((3R,4S)-3-aminotetrahydro-2H-1-pyran-4-yl)-N-benzyl-3-bromo-5-chlorothieno[3,2-b]pyridin-7-amine;
2-((3S,4R)-3-aminotetrahydro-2H-pyran-4-yl)-N-benzyl-3-bromo-5-chlorothieno[3,2-b]pyridin-7-amine;
2-((1R,6R)-6-aminocyclohex-3-en-1-yl)-5-chloro-N-(furan-2-ylmethyl)-3-methylthieno[3,2-b]pyridin-7-amine;
2-((1R,6S)-6-amino-2,2-difluorocyclohexyl)-N-(but-2-yn-1-yl)-5-chloro-3-iodothieno[3,2-b]pyridin-7-amine;
2-((1S,6R)-6-amino-2,2-difluorocyclohexyl)-N-(but-2-yn-1-yl)-5-chloro-3-iodothieno[3,2-b]pyridin-7-amine;
2-((3S,4-R)-3-aminotetrahydro-2H-pyran-4-yl)-3-bromo-5-chloro-1-methyl-N-(thiophen-2-ylmethyl)-1H-pyrrolo[3,2-b]pyridin-7-amine;
6-((1R,6R)-6-aminocyclohex-3-en-1-yl)-7-bromo-2-chloro-N-(thiophen-2-ylmethyl)thieno[3,2-d]pyrimidin-4-amine;
2-((1R,6R)-6-aminocyclohex-3-en-1-yl)-N-(but-2-yn-1-yl)-5-chloro-3-iodothieno[3,2-b]pyridin-7-amine;
5-chloro-3-iodo-2-((1R,6R)-6-(methylamino)cyclohex-3-en-1-yl)-N-(thiophen-2-ylmethyl)thieno[3,2-b]pyridin-7-amine;
5-chloro-3-iodo-2-((1S,6S)-6-(methylamino)cyclohex-3-en-1-yl)-N-(thiophen-2-ylmethyl)thieno[3,2-b]pyridin-7-amine;
2-((3R,4S)-3-aminotetrahydro-2H-pyran-4-yl)-N-benzyl-3,5-dichlorothieno[3,2-b]pyridin-7-amine;
2-((3S,4R)-3-aminotetrahydro-2H-pyran-4-yl)-N-benzyl-3,5-dichlorothieno[3,2-b]pyridin-7-amine;
2-((3S,4R)-4-aminotetrahydro-2H-pyran-3-yl)-3,5-dichloro-N-(furan-2-ylmethyl)thieno[3,2-b]pyridin-7-amine;
6-((3R,4S)-3-aminotetrahydro-2H-pyran-4-yl)-2-chloro-N-(furan-2-ylmethyl)-7-vinylthieno[3,2-d]pyrimidin-4-amine;
6-((3S,4R)-3-aminotetrahydro-2H-pyran-4-yl)-2-chloro-N-(furan-2-ylmethyl)-7-vinylthieno[3,2-d]pyrimidin-4-amine;
6-((1R,2R)-2-aminocyclohexyl)-2-chloro-5-(difluoromethyl)-N-(furan-2-ylmethyl)-7-iodo-5H-pyrrolo[3,2-d]pyrimidin-4-amine;
6-((2S,3S)-3-aminotetrahydro-2H-pyran-2-yl)-N-benzyl-2,7-dichloro-5-(difluoromethyl)-5H-pyrrolo[3,2-d]pyrimidin-4-amine;
6-((2R,3R)-3-aminotetrahydro-2H-pyran-2-yl)-N-benzyl-2,7-dichloro-5-(difluoromethyl)-5H-pyrrolo[3,2-d]pyrimidin-4-amine;
2-((3R,4S)-3-aminotetrahydro-2H-pyran-4-yl)-5-chloro-7-((thiophen-2-ylmethyl)amino)thieno[3,2-b]pyridine-3-carbonitrile;
2-((3S,4R)-3-aminotetrahydro-2H-pyran-4-yl)-5-chloro-7-((thiophen-2-ylmethyl)amino)thieno[3,2-b]pyridine-3-carbonitrile;
2-((1S,6S)-6-aminocyclohex-3-en-1-yl)-5-chloro-3-((2-methoxypyridin-3-yl)ethynyl)-N-(thiophen-2-ylmethyl)thieno[3,2-b]pyridin-7-amine;
2-((1S,6S)-6-aminocyclohex-3-en-1-yl)-5-chloro-3-(pyridin-4-ylethynyl)-N-(thiophen-2-ylmethyl)thieno[3,2-b]pyridin-7-amine;
6-((1R,6R)-6-aminocyclohex-3-en-1-yl)-7-bromo-2-chloro-N-(furan-2-ylmethyl)thieno[3,2-d]pyrimidin-4-amine;
6-((1R,6R)-6-aminocyclohex-3-en-1-yl)-2,7-dichloro-N-(thiophen-2-ylmethyl)thieno[3,2-d]pyrimidin-4-amine;
6-((1S,6S)-6-aminocyclohex-3-en-1-yl)-2,7-dichloro-N-(thiophen-2-ylmethyl)thieno[3,2-d]pyrimidin-4-amine;
2-((3R,4S)-3-aminotetrahydro-2H-pyran-4-yl)-3-bromo-N-(but-2-yn-1-yl)-5-chlorothieno[3,2-b]pyridin-7-amine;
6-((1S,6S)-6-aminocyclohex-3-en-1-yl)-N-benzyl-2-chloro-5-(difluoromethyl)-7-methyl-5H-pyrrolo[3,2-d]pyrimidin-4-amine;
2-((1S,2S)-2-aminocyclohexyl)-5-chloro-3-(fluoromethyl)-N-(thiophen-2-ylmethyl)thieno[3,2-b]pyridin-7-amine;
2-((1R,2R)-2-aminocyclohexyl)-5-chloro-3-(fluoromethyl)-N-(thiophen-2-ylmethyl)thieno[3,2-b]pyridin-7-amine;
2-((1R,2R)-2-aminocyclohexyl)-5-chloro-N-(furan-2-ylmethyl)-3-methylthieno[3,2-b]pyridin-7-amine;
2-((1R,2S,6R)-6-amino-2-methylcyclohex-3-en-1-yl)-3-bromo-5-chloro-N-(thiophen-2-ylmethyl)thieno[3,2-b]pyridin-7-amine;
2-((1S,2R,6S)-6-amino-2-methylcyclohex-3-en-1-yl)-3-bromo-5-chloro-N-(thiophen-2-ylmethyl)thieno[3,2-b]pyridin-7-amine;
2-((1R,6R)-6-aminocyclohex-3-en-1-yl)-N-benzyl-3-bromo-5-chlorothieno[3,2-b]pyridin-7-amine;
2-((1R,6R)-6-aminocyclohex-3-en-1-yl)-N-benzyl-3,5-dichlorothieno[3,2-b]pyridin-7-amine;
2-((3S,4R)-4-aminotetrahydro-2H-pyran-3-yl)-3,5-dichloro-N-(thiophen-2-ylmethyl)thieno[3,2-b]pyridin-7-amine;
2-((3S,4R)-4-aminotetrahydro-2H-pyran-3-yl)-N-benzyl-3,5-dichlorothieno[3,2-b]pyridin-7-amine;
2-((3S,4R)-3-aminotetrahydro-2H-pyran-4-yl)-3,5-dichloro-N-(thiazol-2-ylmethyl)thieno[3,2-b]pyridin-7-amine;
2-(3S,4R)-3-aminotetrahydro-2H-pyran-4-yl)-5-chloro-3-iodo-N-(thiazol-2-ylmethyl)thieno[3,2-b]pyridin-7-amine;
2-((1S,6R)-6-amino-2,2-difluorocyclohexyl)-3,5-dichloro-N-(thiophen-2-ylmethyl)thieno[3,2-b]pyridin-7-amine;
2-((3S,4R)-3-aminotetrahydro-2H-pyran-4-yl)-3,5-dichloro-N-(furan-2-ylmethyl)thieno[3,2-b]pyridin-7-amine;
2-((1R,6R)-6-aminocyclohex-3-en-1-yl)-3-bromo-5-chloro-N-(2-fluorobenzyl)thieno[3,2-b]pyridin-7-amine;
6-((1S,6R)-6-amino-2,2-difluorocyclohexyl)-7-bromo-2-chloro-N-(furan-2-ylmethyl)thieno[3,2-d]pyrimidin-4-amine;
6-((1R,6R)-6-aminocyclohex-3-en-1-yl)-2-chloro-N-(furan-2-ylmethyl)-7-methylthieno[3,2-d]pyrimidin-4-amine;
6-((1R,6R)-6-aminocyclohex-3-en-1-yl)-N-benzyl-2-chloro-7-iodothieno[3,2-d]pyrimidin-4-amine;
2-((1R,6R)-6-aminocyclohex-3-en-1-yl)-3-bromo-5-chloro-N-(thiophen-3-ylmethyl)thieno[3,2-b]pyridin-7-amine;
6-((2R,3R)-3-aminotetrahydro-2H-pyran-2-yl)-2-chloro-5-(difluoromethyl)-7-iodo-N-(thiophen-2-ylmethyl)-5H-pyrrolo[3,2-d]pyrimidin-4-amine;
2-((1R,6R)-6-aminocyclohex-3-en-1-yl)-N-benzyl-3,5-dichloro-1-(difluoromethyl)-1H-pyrrolo[3,2-b]pyridin-7-amine;
6-((1S,2S)-2-aminocyclohexyl)-N-benzyl-2,7-dichloro-5-(difluoromethyl)-5H-pyrrolo[3,2-d]pyrimidin-4-amine;
6-((1R,2R)-2-aminocyclohexyl)-N-benzyl-2,7-dichloro-5-(difluoromethyl)-5H-pyrrolo[3,2-d]pyrimidin-4-amine, 2-((1R,6R)-6-aminocyclohex-3-en-1-yl)-5-chloro-3-methyl-N-(thiophen-3-ylmethyl)thieno[3,2-b]pyridin-7-amine;
2-((1S,6S)-6-aminocyclohex-3-en-1-yl)-N-benzyl-3,5-dichloro-1-(difluoromethyl)-1H-pyrrolo[3,2-b]pyridin-7-amine;
2-((1R,2R)-2-aminocyclohexyl)-3,5-dichloro-1-(difluoromethyl)-N-(thiophen-2-ylmethyl)-1H-pyrrolo[3,2-b]pyridin-7-amine;
2-((1S,2S)-2-aminocyclohexyl)-3,5-dichloro-1-(difluoromethyl)-N-(thiophen-2-ylmethyl)-1H-pyrrolo[3,2-b]pyridin-7-amine;
6-((2R,3R)-3-aminotetrahydro-2H-pyran-2-yl)-2-chloro-5-(difluoromethyl)-N-(furan-2-ylmethyl)-7-iodo-5H-pyrrolo[3,2-d]pyrimidin-4-amine;
6-((2R,3R)-3-aminotetrahydro-2H-pyran-2-yl)-2,7-dichloro-5-(difluoromethyl)-N-(thiophen-2-ylmethyl)-5H-pyrrolo[3,2-d]pyrimidin-4-amine;
6-((1R,6R)-6-aminocyclohex-3-en-1-yl)-N-benzyl-2-chloro-5-(difluoromethyl)-7-iodo-5H-pyrrolo[3,2-d]pyrimidin-4-amine;
2-((1R,6S)-6-amino-2,2-difluorocyclohexyl)-3-bromo-N-((E)-but-2-en-1-yl)-5-chlorothieno[3,2-b]pyridin-7-amine;
2-((1S,6R)-6-amino-2,2-difluorocyclohexyl)-3-bromo-N-((E)-but-2-en-1-yl)-5-chlorothieno[3,2-b]pyridin-7-amine;
2-((2R,3R)-3-aminotetrahydro-2H-pyran-2-yl)-3-bromo-5-chloro-1-(difluoromethyl)-N-(furan-2-ylmethyl)-1H-pyrrolo[3,2-b]pyridin-7-amine;
2-((1R,6R)-6-aminocyclohex-3-en-1-yl)-5-chloro-1-(difluoromethyl)-3-iodo-N-(thiophen-2-ylmethyl)-1H-pyrrolo[3,2-b]pyridin-7-amine;
2-((1R,2R)-2-aminocyclohexyl)-3-bromo-N-(but-2-yn-1-yl)-5-chlorothieno[3,2-b]pyridin-7-amine;
2-((1R,6R)-6-aminocyclohex-3-en-1-yl)-3-bromo-N-(but-2-yn-1-yl)-5-chlorothieno[3,2-b]pyridin-7-amine;
2-((1R,2R)-2-aminocyclohexyl)-5-chloro-3-ethynyl-N-(2-fluorobenzyl)thieno[3,2-b]pyridin-7-amine;
2-((1R,6R)-6-aminocyclohex-3-en-1-yl)-3,5-dichloro-N-(thiophen-2-ylmethyl)thieno[3,2-b]pyridin-7-amine;
2-((1S,6S)-6-aminocyclohex-3-en-1-yl)-3,5-dichloro-N-(thiophen-2-ylmethyl)thieno[3,2-b]pyridin-7-amine;
2-((1R,2R)-2-aminocyclohexyl)-3-bromo-5-chloro-N-(pyridin-4-ylmethyl)thieno[3,2-b]pyridin-7-amine;
2-((1R,6R)-6-aminocyclohex-3-en-1-yl)-3,5-dichloro-N-(pyridin-4-ylmethyl)thieno[3,2-b]pyridin-7-amine;
6-((1R,6R)-6-aminocyclohex-3-en-1-yl)-N-benzyl-7-bromo-2-chlorothieno[3,2-d]pyrimidin-4-amine;
6-((1R,6R)-6-aminocyclohex-3-en-1-yl)-2-chloro-N-((3-fluoropyridin-4-yl)methyl)-7-methylthieno[3,2-d]pyrimidin-4-amine;
6-((1R,6R)-6-aminocyclohex-3-en-1-yl)-2-chloro-7-methyl-N-(pyridin-4-ylmethyl)thieno[3,2-d]pyrimidin-4-amine;
6-((1R,6R)-6-aminocyclohex-3-en-1-yl)-2,7-dichloro-N-(furan-2-ylmethyl)thieno[3,2-d]pyrimidin-4-amine;
2-((1R,6R)-6-amino-3,4-dimethylcyclohex-3-en-1-yl)-3-bromo-N-(but-2-yn-1-yl)-5-chlorothieno[3,2-b]pyridin-7-amine;
6-((1R,6R)-6-aminocyclohex-3-en-1-yl)-2,7-dichloro-N-((3-fluoropyridin-4-yl)methyl)thieno[3,2-d]pyrimidin-4-amine;
6-((1R,6R)-6-aminocyclohex-3-en-1-yl)-2,7-dichloro-N-(pyridin-4-ylmethyl)thieno[3,2-d]pyrimidin-4-amine;
6-((1R,6R)-6-aminocyclohex-3-en-1-yl)-2-chloro-7-methyl-N-(thiazol-2-ylmethyl)thieno[3,2-d]pyrimidin-4-amine;
2-((1S,2S)-2-aminocyclohexyl)-N—((Z)-but-2-en-1-yl)-3,5-dichlorothieno[3,2-b]pyridin-7-amine;
2-((1R,6R)-6-aminocyclohex-3-en-1-yl)-3-chloro-7-((furan-2-ylmethyl)amino)thieno[3,2-b]pyridine-5-carbonitrile;
2-((1R,6R)-6-amino-3,4-dimethylcyclohex-3-en-1-yl)-3,5-dichloro-N-(furan-2-ylmethyl)thieno[3,2-b]pyridin-7-amine;
ethyl (4R,5S)-5-amino-4-(5-chloro-3-methyl-7-((thiophen-2-ylmethyl)amino)thieno[3,2-b]pyridin-2-yl)cyclohex-1-ene-1-carboxylate;
(4S,5S)-5-amino-4-(5-chloro-3-methyl-7-((thiophen-2-ylmethyl)amino)thieno[3,2-b]pyridin-2-yl)cyclohex-1-ene-1-carboxylic acid;
2-((1S,6S)-6-aminocyclohex-3-en-1-yl)-5-chloro-3-((3-chloropyridin-4-yl)ethynyl)-N-(thiophen-2-ylmethyl)thieno[3,2-b]pyridin-7-amine;
2-((1S,6S)-6-aminocyclohex-3-en-1-yl)-5-chloro-N-(thiophen-2-ylmethyl)thieno[3,2-b]pyridin-7-amine;
6-((1R,6R)-6-aminocyclohex-3-en-1-yl-2,2,3,4,5,5-d6)-2-chloro-N-(furan-2-ylmethyl)-7-methylthieno[3,2-d]pyrimidin-4-amine;
2-((1S,2R,5S,6S)-6-amino-2,5-dimethylcyclohex-3-en-1-yl)-3,5-dichloro-N-(furan-2-ylmethyl)thieno[3,2-b]pyridin-7-amine;
5-(2-((1R,6R)-6-aminocyclohex-3-en-1-yl)-5-chloro-7-(methyl(thiophen-2-ylmethyl)amino)thieno[3,2-b]pyridin-3-yl)pent-4-yn-1-ol;
5-(2-((1R,6R)-6-aminocyclohex-3-en-1-yl)-5-chloro-7-((furan-2-ylmethyl)(methyl)amino)thieno[3,2-b]pyridin-3-yl)pent-4-yn-1-ol;
5-(2-((1S,6S)-6-aminocyclohex-3-en-1-yl)-7-(benzyl(methyl)amino)-5-chlorothieno[3,2-b]pyridin-3-yl)pent-4-yn-1-ol;
5-(5-chloro-2-((1R,6R)-6-(dimethylamino)cyclohex-3-en-1-yl)-7-((thiophen-2-ylmethyl)amino)thieno[3,2-b]pyridin-3-yl)pent-4-yn-1-ol;
5-(5-chloro-2-((1S,6S)-6-(dimethylamino)cyclohex-3-en-1-yl)-7-((thiophen-2-ylmethyl)amino)thieno[3,2-b]pyridin-3-yl)pent-4-yn-1-ol;
2-((1R,6R)-6-aminocyclohex-3-en-1-yl)-3-bromo-5-chloro-N-((E)-pent-2-en-1-yl)thieno[3,2-b]pyridin-7-amine; and
2-((1S,6S)-6-aminocyclohex-3-en-1-yl)-3-bromo-5-chloro-N-((E)-pent-2-en-1-yl)thieno[3,2-b]pyridin-7-amine;
wherein the form of the compound is a pharmaceutically acceptable salt, hydrate, solvate, racemate, enantiomer, diastereomer, stereoisomer, tautomer or isotope enriched form thereof.

12. A compound salt or a form thereof, wherein the compound salt is selected from the group consisting of:
2-((2R,3R)-3-aminotetrahydro-2H-pyran-2-yl)-3,5-dichloro-N-(2-fluorobenzyl)thieno[3,2-b]pyridin-7-amine hydrochloride;
2-((2S,3S)-3-aminotetrahydro-2H-pyran-2-yl)-3,5-dichloro-N-(2-fluorobenzyl)thieno[3,2-b]pyridin-7-amine hydrochloride;
2-((2R,3R)-3-aminotetrahydro-2H-pyran-2-yl)-N-benzyl-5-chlorothieno[3,2-b]pyridin-7-amine trifluoroacetate;
2-((2S,3S)-3-aminotetrahydro-2H-pyran-2-yl)-N-benzyl-5-chlorothieno[3,2-b]pyridin-7-amine trifluoroacetate;
2-(3-aminotetrahydro-2H-pyran-2-yl)-N-benzyl-3-bromo-5-chlorothieno[3,2-b]pyridin-7-amine trifluoroacetate;
2-((2R,3R)-3-aminotetrahydro-2H-pyran-2-yl)-5-chloro-N-(2-fluorobenzyl)thieno[3,2-b]pyridin-7-amine trifluoroacetate;
2-(2S,3S)-3-aminotetrahydro-2H-pyran-2-yl)-5-chloro-N-(2-fluorobenzyl)thieno[3,2-b]pyridin-7-amine trifluoroacetate;
2-((2R,3R)-3-aminotetrahydro-2H-pyran-2-yl)-3-bromo-5-chloro-N-(2-fluorobenzyl)thieno[3,2-b]pyridin-7-amine trifluoroacetate;
2-((2S,3S)-3-aminotetrahydro-2H-pyran-2-yl)-3-bromo-5-chloro-N-(2-fluorobenzyl)thieno[3,2-b]pyridin-7-amine trifluoroacetate;
2-((1R,2S)-2-amino-4,4-difluorocyclohexyl)-3-bromo-5-chloro-N-(thiophen-2-ylmethyl)thieno[3,2-b]pyridin-7-amine hydrochloride;
2-((1S,2R)-2-amino-4,4-difluorocyclohexyl)-3-bromo-5-chloro-N-(thiophen-2-ylmethyl)thieno[3,2-b]pyridin-7-amine hydrochloride;
(1R,2S)-2-amino-1-(3,5-dichloro-7-((thiophen-2-ylmethyl)amino)thieno[3,2-b]pyridin-2-yl)cyclohexan-1-ol hydrochloride;
2-((1R,2S)-2-amino-1-fluorocyclohexyl)-3,5-dichloro-N-(thiophen-2-ylmethyl)thieno[3,2-b]pyridin-7-amine hydrochloride;
2-((2S,3S)-3-aminotetrahydro-2H-pyran-2-yl)-5-chloro-N-(2-fluorobenzyl)-3-methylthieno[3,2-b]pyridin-7-amine trifluoroacetate;
2-((1R,2S)-2-amino-1-fluorocyclohexyl)-N-benzyl-3-bromo-5-chlorothieno[3,2-b]pyridin-7-amine hydrochloride;
2-((1R,2S)-2-amino-1-fluorocyclohexyl)-3,5-dichloro-N-(furan-2-ylmethyl)thieno[3,2-b]pyridin-7-amine hydrochloride;
2-((1R,2S)-2-amino-1-fluorocyclohexyl)-5-chloro-N-(furan-2-ylmethyl)-3-methylthieno[3,2-b]pyridin-7-amine hydrochloride;
2-((2R,3S)-3-aminopiperidin-2-yl)-3-bromo-5-chloro-N-(thiophen-2-ylmethyl)thieno[3,2-b]pyridin-7-amine formate;
2-((2S,3R)-3-aminopiperidin-2-yl)-3-bromo-5-chloro-N-(thiophen-2-ylmethyl)thieno[3,2-b]pyridin-7-amine formate;
2-((2R,3R)-3-aminopiperidin-2-yl)-3-bromo-5-chloro-N-(thiophen-2-ylmethyl)thieno[3,2-b]pyridin-7-amine formate;
2-((2S,3S)-3-aminopiperidin-2-yl)-3-bromo-5-chloro-N-(thiophen-2-ylmethyl)thieno[3,2-b]pyridin-7-amine formate;
2-((1R,2R)-2-aminocyclohexyl)-5-chloro-3-iodo-N-(thiophen-2-ylmethyl)thieno[3,2-b]pyridin-7-amine trifluoroacetate;
2-((1S,2S)-2-aminocyclohexyl)-5-chloro-3-methyl-N-(thiophen-2-ylmethyl)thieno[3,2-b]pyridin-7-amine formate;
2-((2R,3R)-3-aminobicyclo[3.2.1]octan-2-yl)-3-bromo-5-chloro-N-(thiophen-2-ylmethyl)thieno[3,2-b]pyridin-7-amine formate;
2-((2S,3S)-3-aminobicyclo[3.2.1]octan-2-yl)-3-bromo-5-chloro-N-(thiophen-2-ylmethyl)thieno[3,2-b]pyridin-7-amine formate;
2-((2R,3R)-3-amino-1-phenylpiperidin-2-yl)-3-bromo-5-chloro-N-(thiophen-2-ylmethyl)thieno[3,2-b]pyridin-7-amine formate;
2-((2S,3S)-3-amino-1-phenylpiperidin-2-yl)-3-bromo-5-chloro-N-(thiophen-2-ylmethyl)thieno[3,2-b]pyridin-7-amine formate;
2-(2R,3S)-3-amino-1-phenylpiperidin-2-yl)-3-bromo-5-chloro-N-(thiophen-2-ylmethyl)thieno[3,2-b]pyridin-7-amine trifluoroacetate;
2-((2S,3R)-3-amino-1-phenylpiperidin-2-yl)-3-bromo-5-chloro-N-(thiophen-2-ylmethyl)thieno[3,2-b]pyridin-7-amine trifluoroacetate;
2-((3R,4S)-3-aminotetrahydro-2H-pyran-4-yl)-3-bromo-5-chloro-N-(thiophen-2-ylmethyl)thieno[3,2-b]pyridin-7-amine trifluoroacetate;
2-((3S,4R)-3-aminotetrahydro-2H-pyran-4-yl)-3-bromo-5-chloro-N-(thiophen-2-ylmethyl)thieno[3,2-b]pyridin-7-amine trifluoroacetate;
2-((3R,4R)-3-aminotetrahydro-2H-pyran-4-yl)-3-bromo-5-chloro-N-(thiophen-2-ylmethyl)thieno[3,2-b]pyridin-7-amine formate;
2-((3S,4S)-3-aminotetrahydro-2H-pyran-4-yl)-3-bromo-5-chloro-N-(thiophen-2-ylmethyl)thieno[3,2-b]pyridin-7-amine formate;
2-((4R,5R)-5-aminoazepan-4-yl)-3-bromo-5-chloro-N-(thiophen-2-ylmethyl)thieno[3,2-b]pyridin-7-amine trifluoroacetate;
2-((4S,5S)-5-aminoazepan-4-yl)-3-bromo-5-chloro-N-(thiophen-2-ylmethyl)thieno[3,2-b]pyridin-7-amine trifluoroacetate;
2-((4R,5S)-5-aminoazepan-4-yl)-3-bromo-5-chloro-N-(thiophen-2-ylmethyl)thieno[3,2-b]pyridin-7-amine trifluoroacetate;
2-((4S,5R)-5-aminoazepan-4-yl)-3-bromo-5-chloro-N-(thiophen-2-ylmethyl)thieno[3,2-b]pyridin-7-amine trifluoroacetate;
2-((1R,2R)-2-amino-5,5-dimethylcyclohexyl)-3-bromo-5-chloro-N-(thiophen-2-ylmethyl)thieno[3,2-b]pyridin-7-amine formate;
2-((1S,2S)-2-amino-5,5-dimethylcyclohexyl)-3-bromo-5-chloro-N-(thiophen-2-ylmethyl)thieno[3,2-b]pyridin-7-amine formate;
2-((2R,3S)-3-aminotetrahydrofuran-2-yl)-3-bromo-5-chloro-N-(thiophen-2-ylmethyl)thieno[3,2-b]pyridin-7-amine formate;
2-((2S,3R)-3-aminotetrahydrofuran-2-yl)-3-bromo-5-chloro-N-(thiophen-2-ylmethyl)thieno[3,2-b]pyridin-7-amine formate;
2-((2R,3R)-3-aminotetrahydrofuran-2-yl)-3-bromo-5-chloro-N-(thiophen-2-ylmethyl)thieno[3,2-b]pyridin-7-amine formate;
2-((2S,3S)-3-aminotetrahydrofuran-2-yl)-3-bromo-5-chloro-N-(thiophen-2-ylmethyl)thieno[3,2-b]pyridin-7-amine formate;
2-((1R,2R)-2-amino-4,4-dimethylcyclohexyl)-3-bromo-5-chloro-N-(thiophen-2-ylmethyl)thieno[3,2-b]pyridin-7-amine formate;
2-((1S,2S)-2-amino-4,4-dimethylcyclohexyl)-3-bromo-5-chloro-N-(thiophen-2-ylmethyl)thieno[3,2-b]pyri-din-7-amine formate;
(1R,5S,6R)-6-amino-5-(3-bromo-5-chloro-7-((thiophen-2-ylmethyl)amino)thieno[3,2-b]pyridin-2-yl)cyclohex-2-en-1-ol formate;
(1S,5R,6S)-6-amino-5-(3-bromo-5-chloro-7-((thiophen-2-ylmethyl)amino)thieno[3,2-b]pyridin-2-yl)cyclohex-2-en-1-ol formate;
5-(2-((1R,6R)-6-aminocyclohex-3-en-1-yl)-5-chloro-7-((thiophen-2-ylmethyl)amino)thiene[3,2-b]pyridin-3-yl)pent-4-yn-1-ol formate;
5-(2-((1S,6S)-6-aminocyclohex-3-en-1-yl)-5-chloro-7-((thiophen-2-ylmethyl)amino)thieno[3,2-b]pyridin-3-yl)pent-4-yn-1-ol formate;
4-(2-((1R,6R)-6-aminocyclohex-3-en-1-yl)-5-chloro-7-((thiophen-2-ylmethyl)amino)thieno[3,2-b]pyridin-3-yl)but-3-yn-1-ol trifluoroacetate;
4-(2-((1S,6S)-6-aminocyclohex-3-en-1-yl)-5-chloro-7-((thiophen-2-ylmethyl)amino)thieno[3,2-b]pyridin-3-yl)but-3-yn-1-ol trifluoroacetate;
6-((1S,6S)-6-aminocyclohex-3-en-1-yl-2,2,3,4,5,5-d6)-7-bromo-2-chloro-N-(thiophen-2-ylmethyl)thieno[3,2-d]pyrimidin-4-amine trifluoroacetate;
2-((1R,6R)-6-aminocyclohex-3-en-1-yl)-3,5-dichloro-N-(thiophen-2-ylmethyl)thieno[3,2-b]pyridin-7-amine formate;
2-((1S,6S)-6-aminocyclohex-3-en-1-yl)-3,5-dichloro-N-(thiophen-2-ylmethyl)thieno[3,2-b]pyri-din-7-amine formate;
6-((1S,6S)-6-aminocyclohex-3-en-1-yl-2,2,3,4,5,5-d6)-2-chloro-N-(furan-2-ylmethyl)-7-methylthieno[3,2-d]pyrimidin-4-amine trifluoroacetate;
2-((1R,2R)-2-aminocyclohexyl)-5-chloro-3-(prop-1-yn-1-yl)-N-(thiophen-2-ylmethyl)thieno[3,2-b]pyridin-7-amine formate;
2-((1S,2S)-2-aminocyclohexyl)-5-chloro-3-(prop-1-yn-1-yl)-N-(thiophen-2-ylmethyl)thieno[3,2-b]pyridin-7-amine formate;
-(2R,3S)-2-amino-3-(3-bromo-5-chloro-7-((thiophen-2-ylmethyl)amino)thieno[3,2-b]pyridin-2-yl)cyclohexan-1-one hydrochloride;
(2S,3R)-2-amino-3-(3-bromo-5-chloro-7-((thiophen-2-ylmethyl)amino)thieno[3,2-b]pyridin-2-yl)cyclohexan-1-one hydrochloride;
2-((1R,6R)-6-aminocyclohex-3-en-1-yl-2,2,3,4,5,5-d6)-3,5-dichloro-N-(furan-2-ylmethyl)thieno[3,2-b]pyridin-7-amine trifluoroacetate;
2-((1S,6S)-6-aminocyclohex-3-en-1-yl-2,2,3,4,5,5-d6)-3,5-dichloro-N-(furan-2-ylmethyl)thieno[3,2-b]pyridin-7-amine trifluoroacetate;
6-((1R,6R)-6-aminocyclohex-3-en-1-yl-2,2,3,4,5,5-d6)-7-bromo-2-chloro-N-(furan-2-ylmethyl)thieno[3,2-d]pyrimidin-4-amine formate;
6-((1S,6S)-6-aminocyclohex-3-en-1-yl-2,2,3,4,5,5-d6)-7-bromo-2-chloro-N-(furan-2-ylmethyl)thieno[3,2-d]pyrimidin-4-amine formate;
2-((3R,4S)-4-aminotetrahydro-2H-pyran-3-yl)-3-bromo-5-chloro-N-(thiophen-2-ylmethyl)thieno[3,2-b]pyridin-7-amine trifluoroacetate;
2-((3S,4R)-4-aminotetrahydro-2H-pyran-3-yl)-3-bromo-5-chloro-N-(thiophen-2-ylmethyl)thieno[3,2-b]pyridin-7-amine trifluoroacetate;
2-((1S,6S)-6-aminocyclohex-3-en-1-yl-2,2,3,4,5,5-d6)-5-chloro-3-methyl-N-(thiophen-2-ylmethyl)thieno[3,2-b]pyridin-7-amine formate;
2-((6R)-6-amino-2,2-difluorocyclohexyl)-5-chloro-3-(prop-1-yn-1-yl)-N-(thiophen-2-ylmethyl)thieno[3,2-b]pyridin-7-amine trifluoroacetate;
2-((6S)-6-amino-2,2-difluorocyclohexyl)-5-chloro-3-(prop-1-yn-1-yl)-N-(thiophen-2-ylmethyl)thieno[3,2-b]pyridin-7-amine trifluoroacetate;
2-((1R,6R)-6-aminocyclohex-3-en-1-yl)-5-chloro-3-iodo-N-(thiophen-2-ylmethyl)thieno[3,2-b]pyridin-7-amine formate;
2-((1S,6S)-6-aminocyclohex-3-en-1-yl)-5-chloro-3-iodo-N-(thiophen-2-ylmethyl)thieno[3,2-b]pyridin-7-amine formate;
(R)-5-(2-((1S,6S)-6-aminocyclohex-3-en-1-yl)-5-chloro-7-((thiophen-2-ylmethyl)amino)thieno[3,2-b]pyridin-3-yl)pent-4-yne-1,2-diol trifluoroacetate;
2-((1S,6S)-6-aminocyclohex-3-en-1-yl-2,2,3,4,5,5-d6)-5-chloro-N-(furan-2-ylmethyl)-3-methylthieno[3,2-b]pyridin-7-amine trifluoroacetate;
4-(2-((2R,3R)-3-aminotetrahydro-2H-pyran-2-1)-5-chloro-7-((thiophen-2-ylmethyl)amino)thieno[3,2-b]pyridin-3-yl)but-3-yn-1-ol trifluoroacetate;
4-(2-((2S,3S)-3-aminotetrahydro-2H-pyran-2-yl)-5-chloro-7-((thiophen-2-ylmethyl)amino)thieno[3,2-b]pyridin-3-yl)but-3-yn-1-ol trifluoroacetate;
(S)-5-(2-(((1S,6S)-6-aminocyclohex-3-en-1-yl)-5-chloro-7-((thiophen-2-ylmethyl)amino)thieno[3,2-b]pyridin-3-yl)pent-4-yne-1,2-diol trifluoroacetate;
2-((1R,2R)-2-aminocyclohexyl)-N-benzyl-5-chloro-3-(prop-1-yn-1-yl)thieno[3,2-b]pyridin-7-amine formate;
2-((1S,2S)-2-aminocyclohexyl)-N-benzyl-5-chloro-3-(prop-1-yn-1-yl)thieno[3,2-b]pyridin-7-amine formate;
2-((3R,4S)-3-aminotetrahydro-2H-pyran-4-yl)-5-chloro-3-(prop-1-yn-1-yl)-N-(thiophen-2-ylmethyl)thieno[3,2-b]pyridin-7-amine formate;
2-((3S,4R)-3-aminotetrahydro-2H-pyran-4-yl)-5-chloro-3-(prop-1-yn-1-yl)-N-(thiophen-2-ylmethyl)thieno[3,2-b]pyridin-7-amine formate;
(1R,2R,3S)-2-amino-3-(3-bromo-5-chloro-7-((thiophen-2-ylmethyl)amino)thieno[3,2-b]pyridin-2-yl)cyclohexan-1-ol trifluoroacetate;
(1S,2S,3R)-2-amino-3-(3-bromo-5-chloro-7-((thiophen-2-ylmethyl)amino)thieno[3,2-b]pyridin-2-yl)cyclohexan-1-ol trifluoroacetate;
6-(2-((1S,6S)-6-aminocyclohex-3-en-1-yl)-5-chloro-7-((thiophen-2-ylmethyl)amino)thieno[3,2-b]pyridin-3-yl)hex-5-yn-1-ol trifluoroacetate;
2-((3R,4S)-3-aminotetrahydro-2H-pyran-4-yl)-3-bromo-5-chloro-N-(furan-2-ylmethyl)thieno[3,2-b]pyridin-7-amine trifluoroacetate;
6-((2R,3R)-3-aminotetrahydro-2H-pyran-2-yl)-2-chloro-N-(furan-2-ylmethyl)-7-iodothieno[3,2-d]pyrimidin-4-amine trifluoroacetate;
6-((2S,3S)-3-aminotetrahydro-2H-pyran-2-yl)-2-chloro-N-(furan-2-ylmethyl)-7-iodothieno[3,2-d]pyrimidin-4-amine trifluoroacetate;
6-((3R,4S)-3-aminotetrahydro-2H-pyran-4-yl)-2-chloro-N-(furan-2-ylmethyl)-7-iodothieno[3,2-d]pyrimidin-4-amine formate;
6-((3S,4R)-3-aminotetrahydro-2H-pyran-4-yl)-2-chloro-N-(furan-2-ylmethyl)-7-iodothieno[3,2-d]pyrimidin-4-amine formate;
3-(2-((2R,3R)-3-aminotetrahydro-2H-pyran-2-yl)-5-chloro-7-((thiophen-2-ylmethyl)amino)thieno[3,2-b]pyridin-3-yl)prop-2-yn-1-ol trifluoroacetate;
3-(2-((2S,3S)-3-aminotetrahydro-2H-pyran-2-yl)-5-chloro-7-((thiophen-2-ylmethyl)amino)thieno[3,2-b]pyridin-3-yl)prop-2-yn-1-ol trifluoroacetate;
3-(6-((2R,3R)-3-aminotetrahydro-2H-pyran-2-yl)-2-chloro-4-((furan-2-ylmethyl)amino)thieno[3,2-d]pyrimidin-7-yl)prop-2-yn-1-ol trifluoroacetate;
3-(6-((2S,3S)-3-aminotetrahydro-2H-pyran-2-yl)-2-chloro-4-((furan-2-ylmethyl)amino)thieno[3,2-d]pyrimidin-7-yl)prop-2-yn-1-ol trifluoroacetate;
2-((2R,3R)-3-amino-5-methylenetetrahydro-2H-pyran-2-yl)-3-bromo-5-chloro-N-(thiophen-2-ylmethyl)thieno[3,2-b]pyridin-7-amine trifluoroacetate;
2-((2S,3S)-3-amino-5-methylenetetrahydro-2H-pyran-2-yl)-3-bromo-5-chloro-N-(thiophen-2-ylmethyl)thieno[3,2-b]pyridin-7-amine trifluoroacetate;
2-((1S,6S)-6-aminocyclohex-3-en-1-yl-2,2,3,4,5,5-d6)-5-chloro-3-methyl-N-(thiophen-2-ylmethyl)thieno[3,2-b]pyridin-7-amine trifluoroacetate;
5-(5-chloro-2-((1S,2S)-2-(methylamino)cyclohexyl)-7-((thiophen-2-ylmethyl)amino)thieno[3,2-b]pyridin-3-yl)pent-4-yn-1-ol trifluoroacetate;
2-((2R,3R)-3-aminotetrahydro-2H-pyran-2-yl)-5-chloro-3-iodo-N-(thiophen-2-ylmethyl)thieno[3,2-b]pyridin-7-amine hydrochloride;
2-((2S,3S)-3-aminotetrahydro-2H-pyran-2-yl)-5-chloro-3-iodo-N-(thiophen-2-ylmethyl)thieno[3,2-b]pyridin-7-amine hydrochloride;
2-((1S,6S)-6-aminocyclohex-3-en-1-yl)-5-chloro-N-(thiophen-2-ylmethyl)-3-vinylthieno[3,2-b]pyridin-7-amine trifluoroacetate;
2-((1R,6S)-6-amino-2,2-difluorocyclohexyl)-3-bromo-5-chloro-N-(thiophen-2-ylmethyl)thieno[3,2-b]pyridin-7-amine trifluoroacetate;
2-((1S,6R)-6-amino-2,2-difluorocyclohexyl)-3-bromo-5-chloro-N-(thiophen-2-ylmethyl)thieno[3,2-b]pyridin-7-amine trifluoeroacetate;
2-((3R,4S)-3-aminotetrahydro-2H-pyran-4-yl)-3,5-dichloro-N-(thiophen-2-ylmethyl)thieno[3,2-b]pyridin-7-amine trifluoroacetate;
2-((3S,4R)-3-aminotetrahydro-2H-pyran-4-yl)-3,5-dichloro-N-(thiophen-2-ylmethyl)thieno[3,2-b]pyridin-7-amine trifluoroacetate;
2-(3R,4S)-3-aminotetrahydro-2H-pyran-4-yl)-5-chloro-3-iodo-N-(thiophen-2-ylmethyl)thieno[3,2-b]pyridin-7-amine formate;
2-((3S,4R)-3-aminotetrahydro-2H-pyran-4-yl)-5-chloro-3-iodo-N-(thiophen-2-ylmethyl)thieno[3,2-b]pyridin-7-amine formate;
2-((2R,3R,5R)-3-amino-5-methyltetrahydro-2H-pyran-2-yl)-3-bromo-5-chloro-N-(thiophen-2-ylmethyl)thieno[3,2-b]pyridin-7-amine trifluoroacetate;
2-((2S,3S,5S)-3-amino-5-methyltetrahydro-2H-pyran-2-yl)-3-bromo-5-Chloro-N-(thiophen-2-ylmethyl)thieno[3,2-b]pyridin-7-amine trifluoroacetate;
5-(5-chloro-2-((1R,6R)-6-(methylamino)cyclohex-3-en-1-yl)-7-((thiophen-2-ylmethyl)amino)thieno[3,2-b]pyridin-3-yl)pent-4-yn-1-ol trifluoroacetate;
5-(5-chloro-2-((1S,6S)-6-(methylamino)cyclohex-3-en-1-yl)-7-((thiophen-2-ylmethyl)amino)thieno[3,2-b]pyridin-3-yl)pent-4-yn-1-ol trifluoroacetate;
2-((1R,3R,4R,6S)-4-aminobicyclo[4.1.0]heptan-3-yl)-3-bromo-5-chloro-N-(thiophen-2-ylmethyl)thieno[3,2-b]pyridin-7-amine trifluoroacetate;
2-((1S,3S,4S,6R)-4-aminobicyclo[4.1.0]heptan-3-yl)-3-bromo-5-chloro-N-(thiophen-2-ylmethyl)thieno[3,2-b]pyridin-7-amine trifluoroacetate;
2-((1S,6S)-6-aminocyclohex-3-en-1-yl-2,2,3,4,5,5-d6)-N-benzyl-5-chloro-3-methylthieno[3,2-b]pyridin-7-amine trifluoroacetate;
5-(2-chloro-6-((1R,6R)-6-(methylamino)cyclohex-3-en-1-yl)-4-((thiophen-2-ylmethyl)amino)thieno[3,2-d]pyrimidin-7-yl)pent-4-yn-1-ol trifluoroacetate;
5-(2-chloro-6-((1S,6S)-6-(methylamino)cyclohex-3-en-1-yl)-4-((thiophen-2-ylmethyl)amino)thieno[3,2-d]pyrimidin-7-yl)pent-4-yn-1-ol trifluoroacetate;
2-((1R,2R)-2-aminocyclohexyl)-N-benzyl-5-chloro-3-iodothieno[3,2-b]pyridin-7-amine formate;
2-((1S,2S)-2-aminocyclohexyl)-N-benzyl-5-chloro-3-iodothieno[3,2-b]pyridin-7-amine formate;
2-((1R,6R)-6-aminocyclohex-3-en-1-yl)-3-bromo-N-((E)-but-2-en-1-yl)-5-chlorothieno[3,2-b]pyridin-7-amine formate;
2-((1S,6S)-6-aminocyclohex-3-en-1-yl)-3-bromo-N-((E)-but-2-en-1-yl)-5-chlorothieno[3,2-b]pyridin-7-amine formate;
2-((1S,6S)-6-aminocyclohex-3-en-1-yl)-N-((E)-but-2-en-1-yl)-3,5-dichlorothieno[3,2-b]pyridin-7-amine formate;
4-(5-chloro-2-((1S,6S)-6-(methylamino)cyclohex-3-en-1-yl)-7-((thiophen-2-ylmethyl)amino)thieno[3,2-b]pyridin-3-yl)but-3-yn-1-ol trifluoroacetate;
2-((1S,6S)-6-aminocyclohex-3-en-1-yl)-5-chloro-3-(pyridin-3-ylethynyl)-N-(thiophen-2-ylmethyl)thieno[3,2-b]pyridin-7-amine trifluoroacetate;
5-(5-chloro-2-((2R,3R)-3-(methylamino)tetrahydro-2H-pyran-2-yl)-7-((thiophen-2-ylmethyl)amino)thieno[3,2-b]pyridin-3-yl)pent-4-yn-1-ol trifluoroacetate;
5-(5-chloro-2-((2S,3S)-3-(methylamine)tetrahydro-2H-pyran-2-yl)-7-((thiophen-2-ylmethyl)amino)thieno[3,2-b]pyridin-3-yl)pent-4-yn-1-cl trifluoroacetate;
2-((3R,4S)-3-aminotetrahydro-2H-pyran-4-yl)-3,5-dichloro-N-(furan-2-ylmethyl)thieno[3,2-b]pyridin-7-amine trifluoroacetate;
2-((3S,4R)-3-aminotetrahydro-2H-pyran-4-yl)-3,5-dichloro-N-(furan-2-ylmethyl)thieno[3,2-b]pyridin-7-amine trifluoracetate;
2-((3R,4S)-3-aminotetrahydro-2H-pyran-4-yl)-3,5-dichloro-N-(thiophen-2-ylmethyl)thieno[3,2-b]pyridin-7-amine trifluoroacetate;
2-((3S,4R)-3-aminotetrahydro-2H-pyran-4-yl)-3,5-dichloro-N-(thiophen-2-ylmethyl)thieno[3,2-b]pyridin-7-amine trifluoroacetate;
2-((3R,4S)-3-aminotetrahydro-2H-pyran-4-yl)-3-bromo-5-chloro-N-(furan-2-ylmethyl)thieno[3,2-b]pyridin-7-amine trifluoroacetate;
2-((3S,4R)-3-aminotetrahydro-2H-pyran-4-yl)-3-bromo-5-chloro-N-(furan-2-ylmethyl)thieno[3,2-b]pyridin-7-amine trifluoroacetate;
6-((3R,4S)-3-aminotetrahydro-2H-pyran-4-yl)-2-chloro-N-(furan-2-ylmethyl)-7-((trimethylsilyl)ethynyl)thieno[3,2-d]pyrimidin-4-amine trifluoroacetate;
6-((3S,4R)-3-aminotetrahydro-2H-pyran-4-yl)-2-chloro-N-(furan-2-ylmethyl)-7-((trimethylsilyl)ethynyl)thieno[3,2-d]pyrimidin-4-amine trifluoroacetate;
2-((3R,4S)-3-aminotetrahydro-2H-pyran-4-yl)-3,5-dichloro-N-(furan-2-ylmethyl)thieno[3,2-b]pyridin-7-amine trifluoroacetate;
2-((3S,4R)-3-aminotetrahydro-2H-pyran-4-yl)-3,5-dichloro-N-(furan-2-ylmethyl)thieno[3,2-b]pyridin-7-amine trifluoroacetate;
7-(2-((1S,6S)-6-aminocyclohex-3-en-1-yl)-5-chloro-7-((thiophen-2-ylmethyl)amino)thieno[3,2-b]pyridin-3-yl)hept-6-yn-1-ol formate;
2-((1S,6S)-6-aminocyclohex-3-en-1-yl-2,2,3,4,5,5-d6)-5-chloro-3-methyl-N-(phenylmethyl-d2)thieno[3,2-b]pyridin-7-amine trifluoroacetate;
2-((1R,6R)-6-aminocyclohex-3-en-1-yl)-5-chloro-3-(propa-1,2-dien-1-yl)-N-(thiophen-2-ylmethyl)thieno[3,2-b]pyridin-7-amine trifluoroacetate;
2-((1S,6S)-6-aminocyclohex-3-en-1-yl)-5-chloro-3-(propa-1,2-dien-1-yl)-N-(thiophen-2-ylmethyl)thieno[3,2-b]pyridin-7-amine trifluoroacetate;
(1R,5R,6S)-5-amino-6-(3-bromo-5-chloro-7-((thiophen-2-ylmethyl)amino)thieno[3,2-b]pyridin-2-yl)cyclohex-2-en-1-ol trifluoroacetate;
(1S,5S,6R)-5-amino-6-(3-bromo-5-chloro-7-((thiophen-2-ylmethyl)amino)thieno[3,2-b]pyridin-2-yl)cyclohex-2-en-1-ol trifluoroacetate;
6-((3R,4S)-3-aminotetrahydro-2H-pyran-4-yl)-2-chloro-7-ethynyl-N-(furan-2-ylmethyl)thieno[3,2-d]pyrimidin-4-amine trifluoroacetate;
6-((3S,4R)-3-aminotetrahydro-2H-pyran-4-yl)-2-chloro-7-ethynyl-N-(furan-2-ylmethyl)thieno[3,2-d]pyrimidin-4-amine trifluoroacetate;
2-((1R,6R)-6-aminocyclohex-3-en-1-yl)-3-bromo-5-chloro-N-(cyclopent-1-en-1-ylmethyl)thieno[3,2-b]pyridin-7-amine formate;
2-((1S,6S)-6-aminocyclohex-3-en-1-yl)-3-bromo-5-chloro-N-(cyclopent-1-en-1-ylmethyl)thieno[3,2-b]pyridin-7-amine formate;
6-((1S,2S)-2-aminocyclohexyl)-2,7-dichloro-5-(difluoromethyl)-N-(furan-2-ylmethyl)-5H-pyrrolo[3,2-d]pyrimidin-4-amine formate;
6-((1R,2R)-2-aminocyclohexyl)-2,7-dichloro-5-(difluoromethyl)-N-(furan-2-ylmethyl)-5H-pyrrolo[3,2-d]pyrimidin-4-amine formate;
6-((3R,4S)-3-aminotetrahydro-2H-pyran-4-yl)-7-bromo-2-chloro-N-(furan-2-ylmethyl)thieno[3,2-d]pyrimidin-4-amine formate;
6-((3S,4R)-3-aminotetrahydro-2H-pyran-4-yl)-7-bromo-2-chloro-N-(furan-2-ylmethyl)thieno[3,2-d]pyrimidin-4-amine formate;
2-((1R,2S,3S)-2-amino-3-fluorocyclohexyl)-5-chloro-3-methyl-N-(thiophen-2-ylmethyl)thieno[3,2-b]pyridin-7-amine trifluoroacetate;
2-((1S,2R,3R)-2-amino-3-fluorocyclohexyl)-5-chloro-3-methyl-N-(thiophen-2-ylmethyl)thieno[3,2-b]pyridin-7-amine trifluoroacetate;
6-((3R,4S)-3-aminotetrahydro-2H-pyran-4-yl)-7-bromo-2-chloro-N-(thiophen-2-ylmethyl)thieno[3,2-d]pyrimidin-4-amine formate;
6-((3S,4R)-3-aminotetrahydro-2H-pyran-4-yl)-7-bromo-2-chloro-N-(thiophen-2-ylmethyl)thieno[3,2-d]pyrimidin-4-amine formate;
2-((3R,4S)-3-aminotetrahydro-2H-pyran-4-yl)-5-chloro-N-(thiophen-2-ylmethyl)-3-vinylthieno[3,2-b]pyridin-7-amine formate;
2-((3S,4R)-3-aminotetrahydro-2H-pyran-4-yl)-5-chloro-N-(thiophen-2-ylmethyl)-3-vinylthieno[3,2-b]pyridin-7-amine formate;
6-((3R,4S)-3-aminotetrahydro-2H-pyran-4-yl)-2,7-dichloro-N-(furan-2-ylmethyl)thieno[3,2-d]pyrimidin-4-amine formate;
6-((3S,4R)-3-aminotetrahydro-2H-pyran-4-yl)-2,7-dichloro-N-(furan-2-ylmethyl)thieno[3,2-d]pyrimidin-4-amine formate;
(1R,2S,3R)-3-amino-2-(3-bromo-5-chloro-7-((thiophen-2-ylmethyl)amino)thieno[3,2-b]pyridin-2-yl)cyclohexan-1-ol trifluoroacetate;
(1S,2R,3S)-3-amino-2-(3-bromo-5-chloro-7-((thiophen-2-ylmethyl)amino)thieno[3,2-b]pyridin-2-yl)cyclohexan-1-ol trifluoroacetate;
2-((1R,6R)-6-amino-2,2-difluorocyclohexyl)-3-bromo-5-chloro-N-(thiophen-2-ylmethyl)thieno[3,2-b]pyridin-7-amine trifluoroacetate;
2-((1S,6S)-6-amino-2,2-difluorocyclohexyl)-3-bromo-5-chloro-N-(thiophen-2-ylmethyl)thieno[3,2-b]pyridin-7-amine trifluoroacetate;
methyl (2-((1S,6S)-6-aminocyclohex-3-en-1-yl)-3-bromo-5-chlorothieno[3,2-b]pyridin-7-yl)glycinate formate;
2-((1R,6S)-6-amino-2,2-difluorocyclohexyl)-3-bromo-N-(but-2-yn-1-yl)-5-chlorothieno[3,2-b]pyridin-7-amine trifluoroacetate;
2-((1S,6R)-6-amino-2,2-difluorocyclohexyl)-3-bromo-N-(but-2-yn-1-yl)-5-chlorothieno[3,2-b]pyridin-7-amine trifluoroacetate;
2-((1R,5S,6S)-6-amino-5-methoxy cyclohex-3-en-1-yl)-3-bromo-5-chloro-N-(thiophen-2-ylmethyl)thieno[3,2-b]pyridin-7-amine hydrochloride;
2-((1S,5R,6R)-6-amino-5-methoxycyclohex-3-en-1-yl)-3-bromo-5-chloro-N-(thiophen-2-ylmethyl)thieno[3,2-b]pyridin-7-amine hydrochloride;
6-((1R,6R)-6-aminocyclohex-3-en-1-yl-2,2,3,4,5,5-d6)-7-bromo-2-chloro-N-(thiophen-2-ylmethyl)thieno[3,2-d]pyrimidin-4-amine trifluoroacetate, (1R,2S,3R)-2-amino-3-(5-chloro-3-methyl-7-((thiophen-2-ylmethyl)amino)thieno[3,2-b]pyridin-2-yl)cyclohexan-1-ol trifluoroacetate;
(1S,2R,3S)-2-amino-3-(5-chloro-3-methyl-7-((thiophen-2-ylmethyl)amino)thieno[3,2-b]pyridin-2-yl)cyclohexan-1-ol trifluoroacetate;
3-bromo-5-chloro-2-((1R,2R)-2-(methylamino)cyclohexyl)-N-(thiophen-2-ylmethyl)thieno[3,2-b]pyridin-7-amine trifluoroacetate;
3-bromo-5-chloro-2-((1S,2S)-2-(methylamino)cyclohexyl)-N-(thiophen-2-ylmethyl)thieno[3,2-b]pyridin-7-amine trifluoroacetate;
(1R,5S,6R)-5-amino-6-(3,5-dichloro-7-((thiophen-2-ylmethyl)amino)thieno[3,2-b]pyridin-2-yl)cyclohex-2-en-1-ol formate;
2-((1S,6S)-6-aminocyclohex-3-en-1-yl)-N-((E)-but-2-en-1-yl)-5-chloro-3-methylthieno[3,2-b]pyridin-7-amine trifluoroacetate;
2-((3R,4S)-3-aminotetrahydro-2H-pyran-4-yl)-N-benzyl-3-bromo-5-chlorothieno[3,2-b]pyridin-7-amine trifluoroacetate;
2-((3S,4R)-3-aminotetrahydro-2H-pyran-4-yl)-N-benzyl-3-bromo-5-chlorothieno[3,2-b]pyridin-7-amine trifluoroacetate;
2-((1R,6S)-6-amino-2,2-difluorocyclohexyl)-N-(but-2-yn-1-yl)-5-chloro-3-iodothieno[3,2-b]pyridin-7-amine trifluoroacetate;
2-((1S,6R)-6-amino-2,2-difluorocyclohexyl)-N-(but-2-yn-1-yl)-5-chloro-3-iodothieno[3,2-b]pyridin-7-amine trifluoroacetate;
5-chloro-3-iodo-2-((1R,6R)-6-(methylamino)cyclohex-3-en-1-yl)-N-(thiophen-2-ylmethyl)thieno[3,2-b]pyridin-7-amine formate;
5-chloro-3-iodo-2-((1S,6S)-6-(methylamino)cyclohex-3-en-1-yl)-N-(thiophen-2-ylmethyl)thieno[3,2-b]pyridin-7-amine formate;
2-((3R,4S)-3-aminotetrahydro-2H-pyran-4-yl)-N-benzyl-3,5-dichlorothieno[3,2-b]pyridin-7-amine trifluoroacetate;
2-((3S,4R)-3-aminotetrahydro-2H-pyran-4-yl)-N-benzyl-3,5-dichlorothieno[3,2-b]pyridin-7-amine trifluoroacetate;
6-((3R,4S)-3-aminotetrahydro-2H-pyran-4-yl)-2-chloro-N-(furan-2-ylmethyl)-7-vinylthieno[3,2-d]pyrimidin-4-amine formate;
6-((3S,4R)-3-aminotetrahydro-2H-pyran-4-yl)-2-chloro-N-(furan-2-ylmethyl)-7-vinylthieno[3,2-d]pyrimidin-4-amine formate;
2-((3R,4S)-3-aminotetrahydro-2H-pyran-4-yl)-5-chloro-7-((thiophen-2-ylmethyl)amino)thieno[3,2-b]pyridine-3-carbonitrile trifluoroacetate;
2-((3S,4R)-3-aminotetrahydro-2H-pyran-4-yl)-5-chloro-7-((thiophen-2-ylmethyl)amino)thieno[3,2-b]pyridine-3-carbonitrile trifluoroacetate;
2-((1S,6S)-6-aminocyclohex-3-en-1-yl)-5-chloro-3-((2-methoxypyridin-3-ylethynyl)-N-(thiophen-2-ylmethyl)thieno[3,2-b]pyridin-7-amine trifluoroacetate;
2-((1S,6S)-6-aminocyclohex-3-en-1-yl)-5-chloro-3-(pyridin-4-ylethynyl)-N-(thiophen-2-ylmethyl)thieno[3,2-b]pyridin-7-amine trifluoroacetate;
2-((1S,2S)-2-aminocyclohexyl)-5-chloro-3-(fluoromethyl)-N-(thiophen-2-ylmethyl)thieno[3,2-b]pyridin-7-amine formate;
2-((1R,2R)-2-aminocyclohexyl)-5-chloro-3-(fluoromethyl)-N-(thiophen-2-ylmethyl)thieno[3,2-b]pyridin-7-amine formate, 2-((3S,4R)-3-aminotetrahydro-2H-pyran-4-yl)-3,5-dichloro-N-(furan-2-ylmethyl)thieno[3,2-b]pyridin-7-amine trifluoroacetate;
2-((1R,2R)-2-aminocyclohexyl)-3,5-dichloro-1-(difluoromethyl)-N-(thiophen-2-ylmethyl)-1H-pyrrolo[3,2-b]pyridin-7-amine formate;
2-((1S,2S)-2-aminocyclohexyl)-3,5-dichloro-1-(difluoromethyl)-N-(thiophen-2-ylmethyl)-1H-pyrrolo[3,2-b]pyridin-7-amine formate;
2-((1R,6S)-6-amino-2,2-difluorocyclohexyl)-3-bromo-N-((E)-but-2-en-1-yl)-5-chlorothieno[3,2-b]pyridin-7-amine trifluoroacetate;
2-((1S,6R)-6-amino-2,2-difluorocyclohexyl)-3-bromo-N-((E)-but-2-en-1-yl)-5-chlorothieno[3,2-b]pyridin-7-amine trifluoroacetate;
2-((1S,2S)-2-aminocyclohexyl)-N—((Z)-but-2-en-1-yl)-3,5-dichlorothieno[3,2-b]pyridin-7-amine trifluoroacetate;
2-((1S,6S)-6-aminocyclohex-3-en-1-yl)-5-chloro-3-((3-chloropyridin-4-yl)ethynyl)-N-(thiophen-2-ylmethyl)thieno[3,2-b]pyridin-7-amine trifluoroacetate;
2-((1S,6S)-6-aminocyclohex-3-en-1-yl)-5-chloro-N-(thiophen-2-ylmethyl)thieno[3,2-b]pyridin-7-amine trifluoroacetate;
6-((1R,6R)-6-aminocyclohex-3-en-1-yl-2,2,3,4,5,5-d6)-2-chloro-N-(furan-2-ylmethyl)-7-methylthieno[3,2-d]pyrimidin-4-amine trifluoroacetate;
5-(5-chloro-2-((1R,6R)-6-(dimethylamino)cyclohex-3-en-1-yl)-7-((thiophen-2-ylmethyl)amino)thieno[3,2-b]pyridin-3-yl)pent-4-yn-1-ol formate; and
5-(5-chloro-2-((1S,6S)-6-(dimethylamino)cyclohex-3-en-1-yl)-7-((thiophen-2-ylmethyl)amino)thieno[3,2-b]pyridin-3-yl)pent-4-yn-1-ol formate;
wherein the form of the compound is a hydrate, solvate, racemate, enantiomer, diastereomer, stereoisomer, tautomer or isotope enriched form thereof.

13. A method of treating spinocerebellar ataxia type 3 comprising administering to a subject in need thereof an effective amount of the compound of claim 1.

14. The method of claim 13, wherein the effective amount of the compound or form thereof induces exon skipping in ATXN3 pre-mRNA in the subject.

15. The method of claim 13, wherein the effective amount of the compound or form thereof is in a range of from about 0.001 mg/kg/day to about 500 mg/kg/day.

16. A pharmaceutical composition comprising an effective amount of the compound of claim 1 in admixture with a pharmaceutically acceptable excipient.


SEQUENCE LISTING (3 sequences)

SEQ ID NO: 1 - DNA, 21 bp, Homo sapiens
  source 1..21: mol_type = other DNA
caacggatttggtcgtattgg

SEQ ID NO: 2 - DNA, 25 bp, Homo sapiens
  source 1..25: mol_type = other DNA
tgatggcaacaatatccactttacc

SEQ ID NO: 3 - DNA, 21 bp, Homo sapiens
  source 1..21: mol_type = other DNA
cgcctggtcaccagggctgct